(19) United States
(12) Patent Application Publication
(10) Pub. No.: US 2011/0257014 A1
(43) Pub. Date: Oct. 20, 2011

(54) 1-(Heteroaryl)pyrazol-4-ylacetic acids, processes for their preparation and their use as herbicides and plant growth regulators

(75) Inventors: Harald Jakobi, Frankfurt (DE); Arianna Martelletti, Sulzbach (DE); Jan Dittgen, Frankfurt (DE); Dieter Feucht, Eschborn (DE); Isolde H?user-Hahn, Leverkusen (DE); Christopher Hugh Rosinger, Hofheim (DE)

(73) Assignee: BAYER CROPSCIENCE AG, Monheim (DE)

(21) Appl. No.: 12/967,705

(22) Filed: Dec. 14, 2010

(30) Foreign Application Priority Data
Dec. 15, 2009 (EP) 09015518.5

(51) Int. Cl. A01N 43/54; A01N 43/56; C07D 417/04; A01N 43/50; A01P 13/00; A01N 43/42; A01N 43/60; C07D 413/04; A01N 43/76; A01P 21/00; C07D 403/04; C07D 401/02

(52) U.S. Cl. 504/239; 548/365.4; 504/280; 548/365.7; 548/312.4; 504/275; 546/167; 504/247; 544/333; 544/405; 504/235; 548/217; 504/270

(57) ABSTRACT

The invention provides compounds of the formula (I) and salts thereof

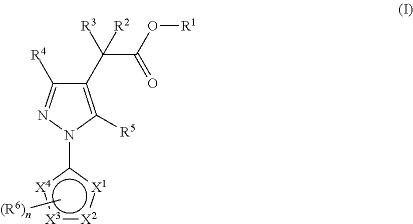

which are described herein. The compounds (I) or salts thereof are suitable for use as herbicides and plant growth regulators, in particular as herbicides for the selective control of harmful plants in crops of useful plants, and can be prepared by the described processes.

The invention relates to the technical field of the herbicides and plant growth regulators, for example the herbicides for controlling broad-leaved weeds and weed grasses in crops of useful plants or the plant growth regulators which can be used for influencing the growth of crop plants.

In their application, crop protection agents known to date for the selective control of harmful plants in crops of useful plants or active compounds for controlling unwanted vegetation sometimes have disadvantages, be it (a) that they have no or else insufficient herbicidal activity against particular harmful plants, (b) that the spectrum of harmful plants which can be controlled with an active compound is not wide enough, (c) that their selectivity in crops of useful plants is too low or that they have a toxicologically unfavorable profile. Other active compounds which can be used as plant growth regulators for a number of useful plants cause unwanted reduced harvest yields in other useful plants or are not compatible with the crop plant, or only within a narrow application rate range. Other known active compounds cannot be produced economically on an industrial scale owing to precursors and reagents which are difficult to obtain, or they have only insufficient chemical stabilities. In the case of other active compounds, the activity is too highly dependent on environmental conditions, such as weather and soil conditions.

EP-A-0822187 and the literature cited therein disclose herbicidal 3-(hetero)aryl-4-[(hetero)arylcarbonyl]pyrazole compounds. Specifically, EP-A-0822187 describes pyrazole compounds which have a phenylcarbonyl radical or a heteroarylcarbonyl group in position 4 and an optionally substituted phenyl radical or heterocyclyl radical in position 5. The compounds described in this publication are not N-substituted at the 1-position (at the nitrogen atom). EP-A-0822187 teaches in a general manner that a removable group may also be present in position 1, and various acyl groups are mentioned by way of example.

U.S. Pat. No. 4,146,721 discloses pyrazolylacetic acids as analgesics, antipyretics and antiinflammatories; however, a use as pesticides, in particular herbicides, is not described.

U.S. Pat. No. 4,095,025 describes 1,3-diarylpyrazol-4-ylacrylic acids and derivatives thereof for pharmaceutical (for example antiinflammatory) purposes.

WO 2004/089931 describes substituted pyrazoles having optionally substituted phenyl or pyrid-3-yl radicals at the nitrogen atom in position 1 of the pyrazole for the treatment and prophylaxis of diseases influenced by the compounds binding to 5 HT receptors.

WO 2008074982 describes pyrazolecarboxamides for use as CB1 receptor modulators.

WO2008080504 describes substituted 1-(3-pyridinyl)pyrazol-4-ylacetic acids as herbicides and plant growth regulators.

Other pyrazol-4-ylacetic acid derivatives which are substituted in the 1-position of the pyrazole radical by an optionally substituted thiazole or isothiazole radical whose ring nitrogen atom is located in the 3-position to the point of attachment have already been proposed as herbicides and plant growth regulators in the international application No. PCT/EP2009/008490 (WO 2010/063422).

For the reasons mentioned, there is still a need for alternative, highly active herbicides for the selective application in plant crops and use on non-crop land. It is also desirable to prepare alternative chemically active compounds which, if appropriate, can be used advantageously as herbicides or plant growth regulators.

The present invention provides compounds of the formula (I) or salts thereof

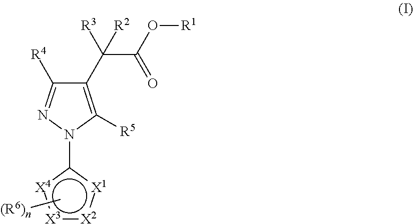

in which
each of the divalent groups X¹, X², X³ and X⁴ is a group of the formula CH, N, NH, O or S, with the proviso that the ring attached at the 1-N atom of the pyrazole ring is a heteroaromatic five-membered ring which is unsubstituted or substituted by radicals R⁶, where in the case of a substitution a hydrogen atom at a group CH or NH is replaced by a radical R⁶,
   except for optionally substituted five-membered heteroaromatic radicals having two heteroatoms as ring atoms in which one of the heteroatoms in the ring is a nitrogen atom and the other is a sulfur atom and the nitrogen atom in the ring is located in the 1,3-position to the ring carbon atom which is attached at the 1-N atom of the pyrazole radical,
R¹ is hydrogen or a hydrolyzable radical, preferably is hydrogen or an optionally substituted hydrocarbon radical or an optionally substituted heterocyclyl radical, where each of the two lastmentioned carbon-containing radicals has, including substituents, 1 to 30 carbon atoms, preferably 1 to 24 carbon atoms, in particular 1 to 20 carbon atoms, or
   a radical of the formula SiR^{a}R^{b}R^{c}, —NR^{a}R^{b} or —N═CR^{c}R^{d},
      where in the 3 lastmentioned formulae each of the radicals R^{a}, R^{b}, R^{c} and R^{d} independently of the others is hydrogen or an optionally substituted hydrocarbon radical or R^{a} and R^{b} together with the nitrogen atom are a 3- to 9-membered heterocycle which, in addition to the nitrogen atom, may contain one or two further ring heteroatoms selected from the group consisting of N, O and S and which is unsubstituted or substituted, or R^{c} and R^{d} together with the carbon atom are a 3- to 9-membered carbocyclic radical or a heterocyclic radical which may contain 1 to 3 ring heteroatoms selected from the group consisting of N, O and S, where the carbocyclic or heterocyclic radical is unsubstituted or substituted,
      where each of the radicals R^{a}, R^{b}, R^{c} and R^{d} including substituents has up to 30 carbon atoms, preferably up to 24 carbon atoms, in particular up to 20 carbon atoms,
R² is hydrogen, halogen or (C₁-C₆)-alkyl which is unsubstituted or substituted by one or more radicals selected from the group consisting of halogen, (C₁-C₄)-alkoxy, (C₁-C₄)-alkylthio and (C₁-C₄)-haloalkoxy,
R³ is hydrogen, halogen or (C₁-C₆)-alkyl which is unsubstituted or substituted by one or more radicals selected from the group consisting of halogen, (C₁-C₄)-alkoxy, (C₁-C₄)-alkylthio and (C₁-C₄)-haloalkoxy, or
R² and R³ together with the carbon atom to which they are attached are a carbocyclic saturated or partially unsaturated ring having 3 to 6 carbon atoms which is unsubstituted or substituted by one or more radicals selected from the group consisting of halogen and (C₁-C₄)-alkyl, and
R⁴ is hydrogen, halogen, cyano, (C₁-C₆)-alkyl, (C₂-C₆)-alkenyl or (C₂-C₆)-alkynyl, where each of the three lastmentioned radicals is unsubstituted or substituted by one or more radicals selected from the group consisting of halogen, cyano, hydroxyl, (C₁-C₄)-alkoxy, (C₁-C₄)-haloalkoxy, (C₁-C₄)-alkoxy-(C₁-C₄)-alkoxy, (C₁-C₄)-alkylthio and optionally halogen-, cyano-, (C₁-C₄)-alkyl- or (C₁-C₄)-haloalkyl-substituted (C₃-C₉)-cycloalkyl or preferably is unsubstituted or substituted by one or more radicals selected from the group consisting of halogen, cyano, (C₁-C₄)-alkoxy, (C₁-C₄)-haloalkoxy, (C₁-C₄)-alkoxy-(C₁-C₄)-alkoxy, (C₁-C₄)-alkylthio and optionally halogen-, cyano-, (C₁-C₄)-alkyl- or (C₁-C₄)-haloalkyl-substituted (C₃-C₉)-cycloalkyl, or
   is (C₃-C₉)-cycloalkyl, (C₅-C₉)-cycloalkenyl or (C₅-C₉)-cycloalkynyl, where each of the 3 lastmentioned radicals is unsubstituted or substituted by one or more radicals selected from the group consisting of halogen, (C₁-C₄)-alkyl, (C₁-C₄)-haloalkyl, (C₂-C₄)-alkenyl, (C₂-C₄)-alkynyl, (C₁-C₄)-alkoxy, (C₁-C₄)-haloalkoxy and (C₁-C₄)-alkylthio, or
   is phenyl which is unsubstituted or substituted by one or more radicals selected from the group consisting of halogen, cyano, nitro, carboxyl, (C₁-C₄)-alkyl, (C₁-C₄)-haloalkyl, (C₂-C₄)-alkenyl, (C₂-C₄)-alkynyl, (C₁-C₄)-alkoxy, (C₁-C₄)-haloalkoxy, (C₁-C₄)-alkoxy-(C₁-C₄)-alkoxy, (C₁-C₄)-alkylthio, (C₁-C₄)-alkanoyl, (C₁-C₄)-haloalkanoyl, [(C₁-C₄)-alkoxy]carbonyl and [(C₁-C₄)-haloalkoxy]carbonyl, or
   is (C₁-C₆)-alkanoyl which is unsubstituted or substituted by one or more radicals selected from the group consisting of halogen, cyano, (C₁-C₄)-alkoxy, (C₁-C₄)-haloalkoxy, (C₁-C₄)-alkoxy-(C₁-C₄)-alkoxy, (C₁-C₄)-alkylthio and optionally halogen-, cyano-, (C₁-C₄)-alkyl- or (C₁-C₄)-haloalkyl-substituted (C₃-C₆)-cycloalkyl, or
   is [(C₁-C₄)-alkoxy]carbonyl which is unsubstituted or substituted by one or more radicals selected from the group consisting of halogen, cyano, (C₁-C₄)-alkoxy, (C₁-C₄)-haloalkoxy, (C₁-C₄)-alkoxy-(C₁-C₄)-alkoxy, (C₁-C₄)-alkylthio and optionally halogen-, cyano-, (C₁-C₄)-alkyl- or (C₁-C₄)-haloalkyl-substituted (C₃-C₆)-cycloalkyl, or
   is [(C₃-C₉)-cycloalkoxy]carbonyl which is unsubstituted or substituted by one or more radicals selected from the group consisting of halogen, (C₁-C₄)-alkyl, (C₁-C₄)-haloalkyl, (C₂-C₄)-alkenyl, (C₂-C₄)-alkynyl, (C₁-C₄)-alkoxy, (C₁-C₄)-haloalkoxy and (C₁-C₄)-alkylthio,
R⁵ is an aryl radical which is unsubstituted or preferably substituted and, including substituents, has 6 to 30 carbon atoms, preferably 6 to 24 carbon atoms, in particular 6 to 20 carbon atoms, or
   a heteroaromatic radical having 1 to 4 ring heteroatoms selected from the group consisting of N, O and S which is unsubstituted or substituted and, including substituents, has 1 to 30 carbon atoms, preferably 1 to 24 carbon atoms, in particular 1 to 20 carbon atoms, and
(R⁶)ₙ are n substituents R⁶, where R⁶, in the case that n=1, or each of the substituents R⁶ independently of the others, in the case that n is greater than 1, is a radical halogen, hydroxyl, amino, nitro, carboxyl, cyano, carbamoyl, (C₁-C₆)-alkyl, (C₁-C₆)-haloalkyl, (C₁-C₄)-alkoxy-(C₁-C₄)-alkyl, (C₁-C₄)-alkylthio-(C₁-C₄)-alkyl, mono- or di-[(C₁-C₄)-alkyl]aminoalkyl, hydroxy-(C₁-C₄)-alkyl, (C₁-C₆)-alkyl which is substituted
   by optionally halogen-, cyano-, (C₁-C₄)-alkyl- or (C₁-C₄)-haloalkyl-substituted (C₃-C₆)-cycloalkyl,
   by optionally halogen-, cyano-, (C₁-C₄)-alkyl-, (C₁-C₄)-haloalkyl- or (C₁-C₄)-alkoxy-substituted phenyl or
   by optionally halogen-, cyano-, (C₁-C₄)-alkyl-, (C₁-C₄)-haloalkyl- or (C₁-C₄)-alkoxy-substituted heterocyclyl, preferably saturated or partially saturated heterocyclyl which contains in each case 3 to 6 ring atoms and one to three ring heteroatoms selected from the group consisting of N, O and S,
   or is (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₄)-alkoxy-(C₁-C₄)-alkoxy, (C₁-C₆)-alkylthio, (C₁-C₆)-haloalkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- or di-[(C₁-C₄)-alkyl]aminocarbonyl, mono- or di-[(C₁-C₆)-acyl]amino, mono- or di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₃-C₉)-cycloalkyl, (C₅-C₉)-cycloalkenyl,
      where each of the two lastmentioned radicals is unsubstituted or substituted by one or more radicals selected from the group consisting of halogen, (C₁-C₄)-alkyl and (C₁-C₄)-haloalkyl,
   or is optionally halogen-, cyano-, (C₁-C₄)-alkyl-, (C₁-C₄)-haloalkyl- or (C₁-C₄)-alkoxy-substituted heterocyclyl, preferably saturated or partially saturated heterocyclyl which contains in each case 3 to 6 ring atoms and one to three ring heteroatoms selected from the group consisting of N, O and S, and
n is 0, 1, 2 or 3.

By addition of a suitable inorganic or organic acid, such as, for example, HCl, HBr, H₂SO₄ or HNO₃, but also oxalic acid or sulfonic acids, onto a basic group, such as, for example, amino or alkylamino, the compounds of the formula (I) may form salts. Suitable substituents present in deprotonated form, such as, for example, sulfonic acids or carboxylic acids, may form inner salts with groups which for their part can be protonated, such as amino groups. Salts may also be formed by replacing the hydrogen of suitable substituents, such as, for example, sulfonic acids or carboxylic acids, by an agriculturally suitable cation. These salts are, for example, metal salts, in particular alkali metal salts or alkaline earth metal salts, especially sodium salts and potassium salts, or else ammonium salts, salts with organic amines or quaternary ammonium salts.

In the formula (I) and all subsequent formulae, terms for chemical radicals are used which have in particular the meanings illustrated below.

A hydrolyzable radical (see definition of R¹) is a radical which can be hydrolyzed under application conditions, for example a radical which can be hydrolyzed even in the spray liquor or in particular under the physiological conditions in plants, where a compound of the formula (I) having the carboxylic ester group —CO—OR¹ (R¹ is not hydrogen) is hydrolyzed to the compound of the formula (I) having the carboxylic acid group —CO—OH (i.e. the compound (I) where R¹═H). Expressly, the definition of the hydrolyzable radicals also includes radicals where R¹=hydrocarbon radical or heterocyclyl radical, the two lastmentioned radicals being unsubstituted or substituted, even if some of them are hydrolyzable comparatively slowly.

A hydrocarbon radical is an aliphatic, cycloaliphatic or aromatic monocyclic or, in the case of an optionally substituted hydrocarbon radical, also a bicyclic or polycyclic organic radical based on the elements carbon and hydrogen, including, for example, the radicals alkyl, alkenyl, alkynyl, cycloalkyl, cycloalkenyl, aryl, phenyl, naphthyl, indanyl, indenyl, etc.; this applies correspondingly to hydrocarbon radicals in composite meanings, such as hydrocarbonoxy radicals or other hydrocarbon radicals attached via heteroatom groups.

Unless defined in more detail, the hydrocarbon radicals preferably have 1 to 20 carbon atoms, more preferably 1 to 16 carbon atoms, in particular 1 to 12 carbon atoms.

The hydrocarbon radicals, also in the special radicals alkyl, alkoxy, haloalkyl, haloalkoxy, alkylamino and alkylthio, and also the corresponding unsaturated and/or substituted radicals may in each case be straight-chain or branched in the carbon skeleton.

The expression “(C₁-C₄)-alkyl” is a brief notation for alkyl having from 1 to 4 carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, for example “(C₁-C₆)-alkyl” correspondingly also include straight-chain or branched alkyl radicals having a larger number of carbon atoms, i.e., according to the example, also the alkyl radicals having 5 and 6 carbon atoms. Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in combined radicals. Alkyl radicals, including in the combined definitions such as alkoxy, haloalkyl, etc., are, for example, methyl, ethyl, n- or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals; alkenyl is, for example, vinyl, allyl, 1-methyl-2-propenyl, 2-methyl-2-propenyl, 2-butenyl, pentenyl, 2-methylpentenyl or hexenyl group, preferably allyl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl. Alkenyl also includes in particular straight-chain or branched hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl.

Alkynyl is, for example, propargyl, but-2-yn-1-yl, but-3-yn-1-yl, 1-methylbut-3-yn-1-yl. Alkynyl also includes, in particular, straight-chain or branched hydrocarbon radicals having more than one triple bond or else having one or more triple bonds and one or more double bonds, for example 1,3-butatrienyl or 3-penten-1-yn-1-yl(pent-3-en-1-yn-1-yl).

A 3- to 9-membered carbocyclic ring is (C₃-C₉)-cycloalkyl or (C₅-C₉)-cycloalkenyl for example.

(C₃-C₉)-Cycloalkyl is a carbocyclic saturated ring system having preferably 3-9 carbon atoms, for example cyclopropyl, cyclobutyl, cyclopentyl, cyclohexyl, cycloheptyl, cyclooctyl or cyclononyl. In the case of substituted cycloalkyl, cyclic systems with substituents are included, where the substituents may also be bonded by a double bond on the cycloalkyl radical, for example an alkylidene group such as methylidene.

(C₅-C₉)-Cycloalkenyl is a carbocyclic, nonaromatic, partially unsaturated ring system having 5-9 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl. In the case of substituted cycloalkenyl, the explanations for substituted cycloalkyl apply correspondingly.

Alkylidene, for example also in the form of (C₁-C₁₀)-alkylidene, is the radical of a straight-chain or branched alkane which is bonded via a double bond, the position of the binding site not being fixed. In the case of a branched alkane, of course, only positions at which two hydrogen atoms may be replaced by the double bond are possible; radicals are, for example, ═CH₂, ═CH—CH₃, ═C(CH₃)—CH₃, ═C(CH₃)—C₂H₅ or ═C(C₂H₅)—C₂H₅.

Halogen is, for example, fluorine, chlorine, bromine or iodine. Haloalkyl, -alkenyl and -alkynyl are, respectively, alkyl, alkenyl and alkynyl substituted partly or fully by identical or different halogen atoms, preferably from the group of fluorine, chlorine and bromine, in particular from the group of fluorine and chlorine, for example monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; the same applies to haloalkenyl and other halogen-substituted radicals.

Aryl is a mono-, bi- or polycyclic carbocyclic aromatic system, for example phenyl, naphthyl, tetrahydronaphthyl, indenyl, indanyl, pentalenyl, fluorenyl and the like, preferably phenyl.

Optionally substituted aryl also includes polycyclic systems, such as tetrahydronaphthyl, indenyl, indanyl, fluorenyl, biphenylyl, where the point of attachment is at the aromatic system.

A heterocyclic radical (heterocyclyl) comprises at least one heterocyclic ring (=carbocyclic ring in which at least one carbon atom is replaced by a heteroatom, preferably by a heteroatom from the group consisting of N, O, S, P, B, Si, Se), which is saturated, unsaturated or heteroaromatic and may be unsubstituted or substituted, where the point of attachment is located at a ring atom.

Unless defined otherwise it preferably contains one or more, in particular 1, 2 or 3, heteroatoms in the heterocyclic ring, preferably from the group consisting of N, O, and S; it is preferably an aliphatic heterocyclyl radical having 3 to 7 ring atoms or a heteroaromatic radical having 5 or 6 ring atoms. The heterocyclic radical may, for example, be a heteroaromatic radical or ring (heteroaryl), such as, for example, a monocyclic, bicyclic or polycyclic aromatic system in which at least 1 ring contains one or more heteroatoms.

If the heterocyclyl radical or the heterocyclic ring is optionally substituted, it can be fused to other carbocyclic or heterocyclic rings. Preference is given to benzo-fused heterocyclic or heteroaromatic rings.

Optionally substituted heterocyclyl also includes polycyclic systems, such as, for example, 8-aza-bicyclo[3.2.1]octanyl or 1-aza-bicyclo[2.2.1]heptyl.

Optionally substituted heterocyclyl also includes spirocyclic systems, such as, for example, 1-oxa-5-aza-spiro[2.3]hexyl.

“Heteroaryl” means, from among the systems defined above under “heterocyclyl”, in each case a heteroaromatic compound, i.e. a fully unsaturated aromatic heterocyclic compound.

The heterocyclic radical is preferably a radical of a heteroaromatic ring having a heteroatom from the group consisting of N, O and S, for example the radical of a five- or six-membered ring, such as pyridyl, pyrrolyl, thienyl or furyl; it is furthermore preferably a radical of a corresponding heteroaromatic ring having 2, 3 or 4 heteroatoms, for example pyrimidinyl, pyridazinyl, pyrazinyl, triazinyl, tetrazinyl, thiazolyl (=1,3-thiazolyl), isothiazolyl (=1,2-thiazolyl), thiadiazolyl, oxazolyl (=1,3-oxazolyl), isoxazolyl (=1,2-oxazolyl), pyrazolyl, imidazolyl or triazolyl or tetrazolyl.

Here, preference is given to a radical of a heteroaromatic five- or six-membered ring having 1 to 4 heteroatoms, such as, for example, 1,2,3-triazolyl, 1,2,4-triazolyl, tetrazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, tetrazolyl, 1,2,3-triazinyl, 1,2,4-triazinyl, 1,3,5-triazinyl, 1,2,3,4-tetrazinyl, 1,2,3,5-tetrazinyl, 1,2,4,5-tetrazinyl, thiazolyl (=1,3-thiazolyl), isothiazolyl (=1,2-thiazolyl), oxazolyl, isoxazolyl, pyrazolyl, imidazolyl.

More preference is given here to heteroaromatic radicals of five-membered heterocycles having 3 nitrogen atoms, such as 1,2,3-triazol-1-yl, 1,2,3-triazol-4-yl, 1,2,3-triazol-5-yl, 1,2,5-triazol-1-yl, 1,2,5-triazol-3-yl, 1,3,4-triazol-1-yl, 1,3,4-triazol-2-yl, 1,2,4-triazol-3-yl, 1,2,4-triazol-5-yl; more preference is also given here to heteroaromatic radicals of six-membered heterocycles having 3 nitrogen atoms, such as 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl;

more preference is also given here to heteroaromatic radicals of five-membered heterocycles having two nitrogen atoms and one oxygen atom, such as 1,2,4-oxadiazol-3-yl; 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl,
more preference is also given here to heteroaromatic radicals of five-membered heterocycles having two nitrogen atoms and one sulfur atom, such as 1,2,4-thiadiazol-3-yl, 1,2,4-thiadiazol-5-yl, 1,3,4-thiadiazol-2-yl, 1,2,3-thiadiazol-4-yl, 1,2,3-thiadiazol-5-yl, 1,2,5-thiadiazol-3-yl;
more preference is also given here to heteroaromatic radicals of five-membered heterocycles having four nitrogen atoms, such as 1,2,3,4-tetrazol-1-yl, 1,2,3,4-tetrazol-5-yl, 1,2,3,5-tetrazol-1-yl, 1,2,3,5-tetrazol-4-yl, 2H-1,2,3,4-tetrazol-5-yl, 1H-1,2,3,4-tetrazol-5-yl,
more preference is also given here to heteroaromatic radicals of six-membered heterocycles such as 1,2,4,5-tetrazin-3-yl;
more preference is also given here to heteroaromatic radicals of five-membered heterocycles having three nitrogen atoms and one oxygen or sulfur atom, such as 1,2,3,4-oxatriazol-5-yl; 1,2,3,5-oxatriazol-4-yl; 1,2,3,4-thiatriazol-5-yl; 1,2,3,5-thiatriazol-4-yl;
more preference is also given here to heteroaromatic radicals of six-membered heterocycles such as, for example, 1,2,4,6-thiatriazin-1-yl; 1,2,4,6-thiatriazin-3-yl; 1,2,4,6-thiatriazin-5-yl.

Furthermore preferably, the heterocyclic radical or ring is a partially or fully hydrogenated heterocyclic radical having one heteroatom from the group of N, O and S, for example oxiranyl, oxetanyl, oxolanyl (=tetrahydrofuryl), oxanyl, pyrrolinyl, pyrrolidyl or piperidyl.

It is also preferably a partially or fully hydrogenated heterocyclic radical having 2 heteroatoms from the group of N, O and S, for example piperazinyl, dioxolanyl, oxazolinyl, isoxazolinyl, oxazolidinyl, isoxazolidinyl and morpholinyl. Suitable substituents for a substituted heterocyclic radical are the substituents specified later on below, and additionally also oxo. The oxo group may also occur on the ring heteroatoms which are able to exist in different oxidation states, as in the case of N and S, for example.

Preferred examples of heterocyclyl are a heterocyclic radical having from 3 to 6 ring atoms from the group of pyridyl, thienyl, furyl, pyrrolyl, oxiranyl, 2-oxetanyl, 3-oxetanyl, oxolanyl (=tetrahydrofuryl), pyrrolidyl, piperidyl, especially oxiranyl, 2-oxetanyl, 3-oxetanyl or oxolanyl, or is a heterocyclic radical having two or three heteroatoms, for example pyrimidinyl, pyridazinyl, pyrazinyl, triazinyl, 1,3-thiazolyl, isothiazolyl, thiadiazolyl, 1,3-oxazolyl, isoxazolyl, pyrazolyl, triazolyl, piperazinyl, dioxolanyl, oxazolinyl, isoxazolinyl, oxazolidinyl, isoxazolidinyl or morpholinyl.

Preferred heterocyclic radicals are also benzo-fused heteroaromatic rings, for example benzofuryl, benzisofuryl, benzothiophenyl, benzisothiophenyl, isobenzothiophenyl, indolyl, isoindolyl, indazolyl, benzimidazolyl, benzotriazolyl, benzoxazolyl, 1,2-benzisoxazolyl, 2,1-benzisoxazolyl, benzothiazolyl, 1,2-benzisothiazolyl, 2,1-benzisothiazolyl, 1,2,3-benzoxadiazolyl, 2,1,3-benzoxadiazolyl, 1,2,3-benzothiadiazolyl, 2,1,3-benzothiadiazolyl, quinolyl(quinolinyl), isoquinolyl (isoquinolinyl), quinnolinyl, phthalazinyl, quinazolinyl, quinoxalinyl, naphthyridinyl, benzotriazinyl, purinyl, pteridinyl, indolizinyl, benzo-1,3-dioxylyl, 4H-benzo-1,3-dioxinyl and 4H-benzo-1,4-dioxinyl, and, where possible, N-oxides and salts thereof.

Examples of five-membered heteroaromatics and preferably of the carbon-linked heteroaromatic radicals of the formula Q

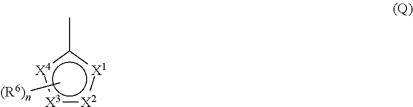

are listed below; what is stated below are the base groups without substituents R⁶; a radical R⁶ may be attached as substituent at the position of a CH or NH group at the ring, in which case it replaces the hydrogen atom at this group:

If the heterocycle contains a single heteroatom, a radical of the formula

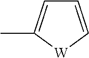

may occur, where the group W is NH, O or S, for example pyrrol-2-yl, furan-2-yl, thien-2-yl.

If the heterocycle contains a single heteroatom, a radical of the formula

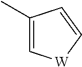

may also occur, where the group W may be NH, O or S, for example pyrrol-3-yl, furan-3-yl, thien-3-yl.

If the heterocycle contains exactly two heteroatoms, in the case of two nitrogen atoms one of the following radicals may occur:

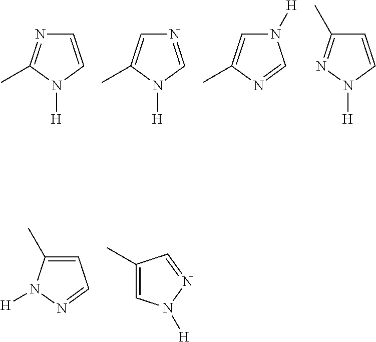

i.e. the radicals 1H-imidazol-2-yl, 1H-imidazol-5-yl, 1H-imidazol-4-yl, 1H-pyrazol-3-yl, 1H-pyrazol-5-yl and 1H-pyrazol-4-yl.

In the case of one nitrogen and one oxygen atom, one of the following radicals may occur:

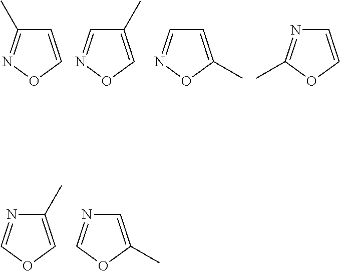

i.e. isoxazol-3-yl (=1,2-oxazol-3-yl), isoxazol-4-yl (=1,2-oxazol-4-yl), isoxazol-5-yl (=1,2-oxazol-5-yl), 1,3-oxazol-2-yl, 1,3-oxazol-4-yl and 1,3-oxazol-5-yl.

In the case of one nitrogen and one sulfur atom, one of the following radicals may occur:

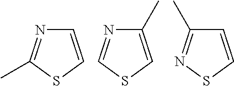

i.e. 1,3-thiazol-2-yl, 1,3-thiazol-4-yl and isothiazol-3-yl (=1,2-thiazol-3-yl); the trivial name “thiazole” (without the positions of the heteroatoms being stated) refers—unless stated otherwise—to 1,3-thiazole, and the trivial name “isothiazole” refers to 1,2-thiazole.

If the heterocycle contains exactly three heteroatoms, in the case of three nitrogen atoms one of the following radicals may occur:

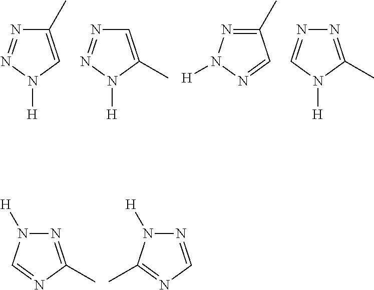

i.e. the radicals 1H-1,2,3-triazol-4-yl, 1H-1,2,3-triazol-5-yl, 2H-1,2,3-triazol-4-yl, 4H-1,2,4-triazol-3-yl, 1H-1,2,4-triazol-3-yl and 1H-1,2,4-triazol-5-yl.

In the case of two nitrogen atoms and one oxygen atom, the following radicals are possible:

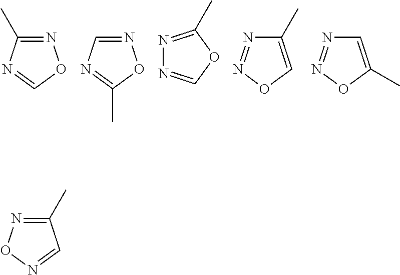

i.e. 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl and 1,2,5-oxadiazol-3-yl;
and in the case of two nitrogen atoms and one sulfur atom the radicals:

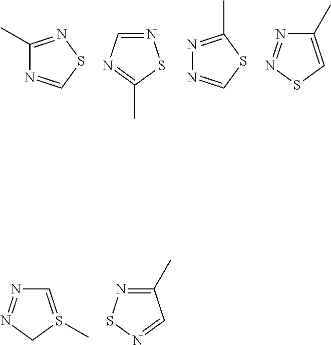

i.e. the radicals 1,2,4-thiadiazol-3-yl, 1,2,4-thiadiazol-5-yl, 1,3,4-thiadiazol-2-yl, 1,2,3-thiadiazol-4-yl, 1,2,3-thiadiazol-5-yl and 1,2,5-thiadiazol-3-yl.

If the heteroaromatic contains exactly four heteroatoms, in the case of four nitrogen atoms one of the following radicals may occur:

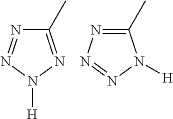

i.e. 2H-1,2,3,4-tetrazol-5-yl and 1H-1,2,3,4-tetrazol-5-yl;
and in the case of three nitrogen atoms and one oxygen or sulfur atom:

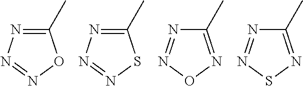

i.e. 1,2,3,4-oxatriazol-5-yl, 1,2,3,4-thiatriazol-5-yl, 1,2,3,5-oxatriazol-4-yl and 1,2,3,5-thiatriazol-4-yl;

Preferred examples of “heteroaryl” containing ring heteroatoms from the group consisting of N, O and S are (nomenclature and numbering of the positions of attachment is in accordance with IUPAC nomenclature):

With one heteroatom:

aromatic five-membered heterocycle radicals such as, for example, 1H-pyrrol-1-yl; 1H-pyrrol-2-yl; 1H-pyrrol-3-yl; furan-2-yl; furan-3-yl; thien-2-yl; thien-3-yl aromatic six-membered heterocycle radicals such as pyridin-2-yl; pyridin-3-yl; pyridin-4-yl.

With two nitrogen atoms:

aromatic five-membered heterocycle radicals such as, for example, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl;
aromatic six-membered heterocycle radicals such as pyrazin-2-yl; pyrazin-3-yl; pyrimidin-2-yl; pyrimidin-4-yl; pyrimidin-5-yl; pyridazin-3-yl; pyridazin-4-yl.

With one nitrogen atom and one oxygen atom:

aromatic five-membered heterocycles such as, for example,
isoxazol-3-yl; isoxazol-4-yl; isoxazol-5-yl; 1,3-oxazol-2-yl; 1,3-oxazol-4-yl; 1,3-oxazol-5-yl.

With one nitrogen atom and one sulfur atom:

aromatic five-membered heterocycles such as, for example, isothiazol-3-yl;
isothiazol-4-yl; isothiazol-5-yl; 1,3-thiazol-2-yl; 1,3-thiazol-4-yl; 1,3-thiazol-5-yl.

With three nitrogen atoms:

aromatic five-membered heterocycles such as, for example, 1H-1,2,3-triazol-1-yl; 1H-1,2,3-triazol-4-yl; 1H-1,2,3-triazol-5-yl; 2H-1,2,3-triazol-2-yl; 2H-1,2,3-triazol-4-yl; 1H-1,2,4-triazol-1-yl; 1H-1,2,4-triazol-3-yl; 4H-1,2,4-triazol-4-yl;
aromatic six-membered heterocycles such as 1,3,5-triazin-2-yl; 1,2,4-triazin-3-yl; 1,2,4-triazin-5-yl; 1,2,4-triazin-6-yl; 1,2,3-triazin-4-yl; 1,2,3-triazin-5-yl.

With two nitrogen atoms and one oxygen atom:

aromatic five-membered heterocycles such as, for example, 1,2,4-oxadiazol-3-yl,
1,2,4-oxadiazol-5-yl, 1,3,4-oxadiazol-2-yl, 1,2,3-oxadiazol-4-yl, 1,2,3-oxadiazol-5-yl and 1,2,5-oxadiazol-3-yl.

With two nitrogen atoms and one oxygen atom:

aromatic five-membered heterocycles such as, for example, 1,2,4-thiadiazol-3-yl; 1,2,4-thiadiazol-5-yl; 1,3,4-thiadiazol-2-yl; 1,2,3-thiadiazol-4-yl; 1,2,3-thiadiazol-5-yl; 1,2,5-thiadiazol-3-yl.

With four nitrogen atoms:

aromatic five-membered heterocycles such as, for example, 1H-1,2,3,4-tetrazol-1-yl; 1H-1,2,3,4-tetrazol-5-yl; 2H-1,2,3,4-tetrazol-2-yl; 2H-1,2,3,4-tetrazol-5-yl aromatic six-membered heterocycle radicals such as 1,2,4,5-tetrazin-3-yl.

With three nitrogen atoms and one oxygen atom or sulfur atom:

aromatic five-membered heterocycles such as, for example, 1,2,3,4-oxatriazol-5-yl; 1,2,3,5-oxatriazol-4-yl; 1,2,3,4-thiatriazol-5-yl; 1,2,3,5-thiatriazol-4-yl;
aromatic six-membered heterocycles such as, for example, 1,2,4,6-thiatriazin-1-yl; 1,2,4,6-thiatriazin-3-yl; 1,2,4,6-thiatriazin-5-yl.

When a base structure is substituted “by one or more radicals” from a list of radicals (=group) or a generically defined group of radicals, this in each case includes simultaneous substitution by a plurality of identical and/or structurally different radicals.

Substituted radicals, such as a substituted alkyl, alkenyl, alkynyl, cycloalkyl, aryl, phenyl, benzyl, heterocyclyl and heteroaryl radical, are, for example, a substituted radical derived from the unsubstituted base structure, where the substituents are, for example, one or more, preferably 1, 2 or 3, radicals selected from the group of halogen, alkoxy, alkylthio, hydroxyl, amino, nitro, carboxyl, cyano, azido, alkoxycarbonyl, alkylcarbonyl, formyl, carbamoyl, mono- and dialkylaminocarbonyl, substituted amino such as acylamino, mono- and dialkylamino, and alkylsulfinyl, alkylsulfonyl and, in the case of cyclic radicals, also alkyl, haloalkyl, alkylthioalkyl, alkoxyalkyl, optionally substituted mono- and dialkylaminoalkyl and hydroxyalkyl; in the term “substituted radicals”, such as the substituted alkyl, alkenyl, alkynyl, cycloalkyl, aryl, phenyl, benzyl, heterocyclyl and heteroaryl radicals mentioned, substituents include, in addition to the saturated hydrocarbon radicals mentioned such as alkyl, alkoxy, alkylthio, alkoxycarbonyl, haloalkyl, cycloalkyl or cycloalkyloxy, corresponding unsaturated aliphatic and aromatic radicals, such as optionally substituted alkenyl, alkynyl, alkenyloxy, alkynyloxy, phenyl and phenoxy. In the case of substituted cyclic radicals having aliphatic moieties in the ring, cyclic systems with those substituents which are bonded on the ring by a double bond are also included, for example substituted by an alkylidene group such as methylidene or ethylidene.

The term “radicals selected from the group consisting of (followed by the group=list of the substituents)” is, wherever used, meant to be synonymous with “radicals selected from the group ( . . . )” or “radicals selected from the group consisting of ( . . . )”.

The substituents given by way of example (“first substituent level”) can, if they include hydrocarbon-containing fractions, be further substituted therein if desired (“second substituent level”), by for example one of the substituents as defined for the first substituent level. Corresponding further substituent levels are possible. The term “substituted radical” preferably embraces just one or two substituent levels.

“Base radical” refers to the respective base structure of a radical to which substituents of a substituent level are attached.

Preferred substituents for the substituent levels are, for example, amino, hydroxyl, halogen, nitro, cyano, mercapto, carboxyl, carbonamide, SF₅, aminosulfonyl, alkyl, cycloalkyl, alkenyl, cycloalkenyl, alkynyl, monoalkylamino, dialkylamino, N-alkanoylamino, alkoxy, alkenyloxy, alkynyloxy, cycloalkoxy, cycloalkenyloxy, alkoxycarbonyl, alkenyloxycarbonyl, alkynyloxycarbonyl, aryloxycarbonyl, alkanoyl, alkenylcarbonyl, alkynylcarbonyl, arylcarbonyl, alkylthio, cycloalkylthio, alkenylthio, cycloalkenylthio, alkynylthio, alkylsulfinyl, alkylsulfonyl, monoalkylaminosulfonyl, dialkylaminosulfonyl, N-alkylaminocarbonyl, N,N-dialkyl-aminocarbonyl, N-alkanoylaminocarbonyl, N-alkanoyl-N-alkylaminocarbonyl, aryl, aryloxy, benzyl, benzyloxy, benzylthio, arylthio, arylamino and benzylamino. Two substituents together may also form a saturated or unsaturated hydrocarbon bridge or a corresponding bridge in which carbon atoms, CH groups or CH₂ groups are replaced by heteroatoms, thus forming a fused-on or fused cycle. Here, with preference benzo-fused systems based on the base structure are formed.

Optionally substituted phenyl is preferably phenyl or phenyl which is unsubstituted or substituted by one or more radicals selected from the group consisting of halogen, cyano, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkoxy-(C₁-C₄)-alkoxy, (C₁-C₄)-alkylthio and nitro, in particular phenyl which is optionally substituted by one or more radicals selected from the group consisting of halogen, (C₁-C₄)-alkyl, (C₁-C₄)-haloalkyl and (C₁-C₄)-alkoxy.

In the case of radicals having carbon atoms preference is given to those having 1 to 6 carbon atoms, preferably 1 to 4 carbon atoms, particularly 1 or 2 carbon atoms. Preference is generally given to substituents selected from the group consisting of halogen, for example fluorine and chlorine, (C₁-C₄)-alkyl, preferably methyl or ethyl, (C₁-C₄)-haloalkyl, preferably trifluoromethyl, (C₁-C₄)-alkoxy, preferably methoxy or ethoxy, (C₁-C₄)-haloalkoxy, nitro and cyano. Particular preference is given here to the substituents methyl, methoxy, fluorine and chlorine.

Substituted amino, such as mono- or disubstituted amino, is a radical from the group of the substituted amino radicals which are N-substituted, for example, by one or two identical or different radicals selected from the group of alkyl, alkoxy, acyl and aryl; preferably mono- and dialkylamino, mono- and diarylamino, acylamino, N-alkyl-N-arylamino, N-alkyl-N-acylamino and N-heterocycles; preference is given to alkyl radicals having from 1 to 4 carbon atoms; aryl is preferably phenyl or substituted phenyl; acyl is as defined below, preferably (C₁-C₄)-alkanoyl. The same applies to substituted hydroxylamino or hydrazino.

Acyl is a radical of an organic acid which arises in a formal sense by removal of a hydroxyl group on the acid function, and the organic radical in the acid may also be bonded to the acid function via a heteroatom. Examples of acyl are the —CO—R radical of a carboxylic acid HO—CO—R and radicals of acids derived therefrom, such as those of thiocarboxylic acid, optionally N-substituted iminocarboxylic acids or the radical of carbonic monoesters, N-substituted carbamic acid, sulfonic acids, sulfinic acids, N-substituted sulfonamide acids, phosphonic acids or phosphinic acids.

Acyl is, for example, formyl, alkylcarbonyl such as [(C₁-C₄)-alkyl]carbonyl, phenylcarbonyl, alkyloxycarbonyl, phenyloxycarbonyl, benzyloxycarbonyl, alkylsulfonyl, alkylsulfinyl, N-alkyl-1-iminoalkyl and other radicals of organic acids. The radicals may each be substituted further in the alkyl or phenyl moiety, for example in the alkyl moiety by one or more radicals selected from the group of halogen, alkoxy, phenyl and phenoxy; examples of substituents in the phenyl moiety are the substituents already mentioned above in general for substituted phenyl. Acyl is preferably an acyl radical in the narrower sense, i.e. a radical of an organic acid in which the acid group is bonded directly to the carbon atom of an organic radical, for example formyl, alkylcarbonyl such as acetyl or [(C₁-C₄)-alkyl]carbonyl, phenylcarbonyl, alkylsulfonyl, alkylsulfinyl and other radicals of organic acids. More preferably, acyl is an alkanoyl radical having 1 to 6 carbon atoms, in particular 1 to 4 carbon atoms. Here, (C₁-C₄)-alkanoyl is the radical of an alkanoic acid having 1 to 4 carbon atoms formed after removal of the OH group of the acid group, i.e. formyl, acetyl, n-propionyl, i-propionyl or n-, i-, sec- or tert-butanoyl.

The “yl position” of a radical denotes the carbon atom having the free bond. Compounds of the formula (I) according to the invention and compounds of the formula (I) used according to the invention (and, if appropriate, salts thereof) are in short also referred to as “compounds (I)”.

The invention also provides all stereoisomers which are encompassed by formula (I) and mixtures thereof. Such compounds of the formula (I) contain one or more asymmetric carbon atoms or else double bonds which are not stated specifically in the formulae (I). The possible stereoisomers defined by their specific three-dimensional shape, such as enantiomers, diastereomers, Z- and E-isomers, are all encompassed by the formula (I) and can be obtained from mixtures of the stereoisomers by customary methods or else prepared by stereoselective reactions in combination with the use of stereochemically pure starting materials.

The invention also provides all tautomers of the compounds of the formula (I) which may result from a hydrogen shift (for example keto-enol tautomers). The compound of the formula (I) also includes the tautomers, even if formally the formula (I) correctly describes only one of the respective tautomers which are in an equilibrium with one another or which can be converted into one another.

The compounds of the formula (I) also include all physical forms in which they may be present as a pure substance or, if appropriate, as a mixture with other compounds, in particular also polymorphic crystal forms of the compounds of the formula (I) and salts thereof and solvent adducts (for example hydrates).

Primarily for reasons of higher herbicidal activity, better selectivity and/or better producibility, compounds of the abovementioned formula (I) according to the invention or their salts or their use according to the invention are of particular interest in which individual radicals have one of the preferred meanings already specified or specified below, or in particular those in which one or more of the preferred meanings already specified or specified below occur in combination.

Irrespective of the respective other radicals and symbols from the group consisting of R¹, R², R³, R⁴, R⁵, (R⁶)ₙ, X¹, X², X³, X⁴ and n and the subdefinitions corresponding to the general radicals, and preferably in combination with preferred definitions of one or more of these radicals, compounds according to the invention or uses according to the invention of compounds of particular interest are those with the preferred meanings listed below of the radicals in question.

Preference is given to the compounds of the formula (I) according to the invention or salts thereof in which
R¹ is hydrogen, alkyl, alkenyl, alkynyl, cycloalkyl, cycloalkenyl, cycloalkynyl or aryl, where each of the 7 lastmentioned radicals is unsubstituted or substituted and, including substituents, has up to 30 carbon atoms, preferably up to 24 carbon atoms, in particular up to 20 carbon atoms, or a heterocyclyl radical having 3 to 9 ring atoms which contains 1 to 4 heteroatoms from the group consisting of N, O and S, which is unsubstituted or substituted and which, including substituents, has 1 to 30 carbon atoms, preferably 1 to 24 carbon atoms, in particular 1 to 20 carbon atoms.

Here, more preference is also given to compounds (I) or salts thereof in which
R¹ is hydrogen.

Here, more preference is also given to compounds (I) or salts thereof in which
R¹ is H, (C₁-C₁₈)-alkyl, (C₂-C₁₈)-alkenyl, (C₂-C₁₈)-alkynyl, (C₃-C₉)-cycloalkyl, (C₅-C₉)-cycloalkenyl, (C₅-C₉)-cycloalkynyl or phenyl, where each of the 7 lastmentioned radicals is unsubstituted or substituted and, including substituents, has up to 30 carbon atoms, preferably up to 24 carbon atoms, in particular up to 20 carbon atoms.

Here, more preference is also given to compounds (I) or salts thereof in which
R¹ is H, (C₁-C₁₈)-alkyl, (C₂-C₁₈)-alkenyl, (C₂-C₁₈)-alkynyl, (C₃-C₉)-cycloalkyl, (C₅-C₉)-cycloalkenyl, (C₅-C₉)-cycloalkynyl or phenyl,
   where each of the 7 lastmentioned radicals is unsubstituted or substituted by one or more radicals selected from the group consisting of halogen, cyano, thio, nitro, hydroxyl, carboxyl, (C₁-C₈)-alkyl, (C₁-C₈)-haloalkyl, (C₁-C₄)-alkoxy-(C₁-C₄)-alkyl, (C₂-C₈)-alkenyl, (C₂-C₈)-haloalkenyl, (C₂-C₈)-alkynyl, (C₂-C₈)-haloalkynyl, the 7 lastmentioned radicals only in the case of cyclic base radicals, (C₁-C₈)-alkoxy, (C₂-C₈)-alkenyloxy, (C₂-C₈)-alkynyloxy, (C₁-C₈)-haloalkoxy, (C₁-C₄)-alkoxy-(C₁-C₄)-alkoxy, (C₁-C₈)-alkylthio, (C₂-C₈)-alkenylthio, (C₂-C₈)-alkynylthio, radicals of the formulae —NR*R**, —CO—NR*R** and —O—CO—NR*R**,
      where each of the radicals R* and R** in the 3 lastmentioned formulae independently of the others is H, (C₁-C₈)-alkyl, (C₂-C₈)-alkenyl, (C₂-C₈)-alkynyl, benzyl, substituted benzyl, phenyl or substituted phenyl, or together with the nitrogen atom is a 3- to 8-membered heterocycle which, in addition to the nitrogen atom, may contain one or two further ring heteroatoms selected from the group consisting of N, O and S and which is unsubstituted or substituted by one or more radicals selected from the group consisting of (C₁-C₄)-alkyl and (C₁-C₄)-haloalkyl,
   and [(C₁-C₈)-alkoxy]carbonyl, [(C₁-C₈)-alkoxy]thiocarbonyl, [(C₂-C₈)-alkenyloxy]carbonyl, [(C₂-C₈)-alkynyloxy]carbonyl, [(C₁-C₈)-alkylthio]carbonyl, [(C₂-C₈)-alkenylthio]carbonyl, [(C₂-C₈)-alkynylthio]carbonyl, (C₁-C₈)-alkanoyl, [(C₂-C₈)-alkenyl]carbonyl, [(C₂-C₈)-alkynyl]carbonyl, (C₁-C₄)-alkylimino, (C₁-C₄)-alkoxyimino, [(C₁-C₈)-alkyl]carbonylamino, [(C₂-C₈)-alkenyl]carbonylamino, [(C₂-C₈)-alkynyl]carbonylamino, [(C₁-C₈)-alkoxy]carbonylamino, [(C₂-C₈)-alkenyloxy]carbonylamino, [(C₂-C₈)-alkynyloxy]carbonylamino, [(C₁-C₈)-alkylamino]carbonylamino, [(C₁-C₆)-alkyl]carbonyloxy, [(C₂-C₆)-alkenyl]carbonyloxy, [(C₂-C₆)-alkynyl]carbonyloxy, [(C₁-C₈)-alkoxy]carbonyloxy, [(C₂-C₈)-alkenyloxy]carbonyloxy, [(C₂-C₈)-alkynyloxy]carbonyloxy, (C₁-C₈)-alkylsulfinyl and (C₁-C₈)-alkylsulfonyl,
      where each of the 27 lastmentioned radicals is unsubstituted or substituted by one or more radicals selected from the group consisting of halogen, NO₂, (C₁-C₄)-alkoxy and optionally substituted phenyl,
   and phenyl, phenyl-(C₁-C₆)-alkoxy, phenyl-[(C₁-C₆)-alkoxy]carbonyl, phenoxy, phenoxy-(C₁-C₆)-alkoxy, phenoxy-[(C₁-C₆)-alkoxy]carbonyl, phenoxycarbonyl, phenylcarbonyloxy, phenoxycarbonyloxy, phenylcarbonylamino, phenyl-[(C₁-C₆)-alkyl]carbonylamino, phenyl-[(C₁-C₆)-alkyl]carbonyloxy, phenyl-[(C₁-C₆)-alkoxy]carbonyloxy, (C₃-C₇)-cycloalkyl, (C₃-C₇)-cycloalkoxy, (C₃-C₆)-cycloalkyl-(C₁-C₆)-alkoxy, (C₃-C₆)-cycloalkyl-[(C₁-C₆)-alkoxy]carbonyl, (C₃-C₆)-cycloalkoxy-(C₁-C₆)-alkoxy, (C₃-C₆)-cycloalkoxy-[(C₁-C₆)-alkoxy]carbonyl, (C₃-C₆)-cycloalkoxycarbonyl, (C₃-C₆)-cycloalkylcarbonyloxy, (C₃-C₆)-cycloalkoxycarbonyloxy, (C₃-C₆)-cycloalkyl-[(C₁-C₆)-alkoxy]carbonyloxy, (C₃-C₆)-cycloalkylcarbonylamino, (C₃-C₆)-cycloalkyl-[(C₁-C₆)-alkyl]carbonylamino and (C₃-C₆)-cycloalkyl-[(C₁-C₆)-alkyl]carbonyloxy,
      where each of the 26 lastmentioned radicals is optionally also fused, preferably benzo-fused, with a carbocyclic or heterocyclic ring, preferably a carbocyclic ring having 3 to 6 carbon atoms or a heterocyclic ring having 5 or 6 ring atoms and 1 to 3 ring heteroatoms from the group consisting of N, O and S, and is unsubstituted in the ring or in the polycyclic system or is substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
   and radicals of the formulae —SiR′₃, —O—SiR′₃, (R′)₃Si—(C₁-C₆)-alkoxy, —CO—O—NR′₂, —O—N═CR′₂, —N═CR′₂, —O—NR′₂, —CH(OR′)₂ and —O—(CH₂)ₘ—CH(OR′)₂,
      in which each of the radicals R′ independently of the others is H, (C₁-C₄)-alkyl or phenyl which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro or substituted in two adjacent positions by a (C₂-C₆)-alkylene bridge, and m is an integer of from 0 to 6,
   and radicals of the formula R″O—CHR′″CH(OR″)—(C₁-C₆)-alkoxy,
      in which each of the radicals R″ independently of the others is H or (C₁-C₄)-alkyl or the radicals R″ together are a (C₁-C₆)-alkylene group and R′″ is H or (C₁-C₄)-alkyl,
   and also radicals of the formula Het¹, where Het¹ is in each case independently of the others a saturated, partially unsaturated or heteroaromatic heterocyclyl radical having 3 to 9 ring atoms, preferably 5 or 6 ring atoms, where the heterocyclic radical in question contains 1 to 4 heteroatoms, preferably 1 to 3 ring heteroatoms, selected from the group consisting of N, O and S and is optionally also fused, preferably benzo-fused, with a carbocyclic or heterocyclic ring, preferably a carbocyclic ring having 3 to 6 carbon atoms or a heterocyclic ring having 5 or 6 ring atoms and 1 to 3 ring heteroatoms selected from the group consisting of N, O and S, and is unsubstituted in the ring or in the polycyclic system or is substituted by one or more radicals selected from the group consisting of halogen, cyano, thio, nitro, hydroxyl, carboxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, (C₃-C₆)-cycloalkyl, (C₃-C₆)-cycloalkoxy, [(C₁-C₈)-alkoxy]carbonyl, [(C₁-C₆)-haloalkoxy]carbonyl and oxo,
   or
R¹ is a polycyclic radical based on (C₃-C₉)-cycloalkyl, (C₅-C₉)-cycloalkenyl, (C₅-C₉)-cycloalkynyl or phenyl, where the basic ring is fused, preferably benzo-fused, with a carbocyclic or heterocyclic ring, preferably a 5- or 6-membered ring having 0 or 1 to 3 ring heteroatoms selected from the group consisting of N, O and S, and where the basic ring or the polycyclic system is unsubstituted or substituted by one or more radicals selected from the group consisting of halogen, cyano, thio, nitro, hydroxyl, carboxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, (C₃-C₆)-cycloalkyl, (C₃-C₆)-cycloalkoxy, [(C₁-C₈)-alkoxy]carbonyl, [(C₁-C₆)-haloalkoxy]carbonyl and oxo,
   or
R¹ is a saturated, partially unsaturated or heteroaromatic heterocyclyl radical having 3 to 9 ring atoms, preferably 5 or 6 ring atoms, which contains 1 to 4 heteroatoms, preferably 1 to 3 ring heteroatoms, selected from the group consisting of N, O and S, optionally also fused, preferably benzo-fused, with a carbocyclic or heterocyclic ring, preferably a 5- or 6-membered ring having 0 or 1 to 3 ring heteroatoms from the group consisting of N, O and S, and which is unsubstituted in the ring or in the polycyclic system or is substituted by one or more radicals selected from the group consisting of halogen, cyano, thio, nitro, hydroxyl, carboxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, (C₃-C₆)-cycloalkyl, (C₃-C₆)-cycloalkoxy, [(C₁-C₈)-alkoxy]carbonyl, [(C₁-C₆)-haloalkoxy]carbonyl and oxo.

Here, more preference is also given to compounds (I) or salts thereof in which
R¹ is H, (C₁-C₁₈)-alkyl, (C₂-C₁₈)-alkenyl, (C₂-C₁₈)-alkynyl, (C₃-C₉)-cycloalkyl, (C₅-C₉)-cycloalkenyl, (C₅-C₉)-cycloalkynyl or phenyl,
   where each of the 7 lastmentioned radicals is unsubstituted or substituted by one or more radicals selected from the group consisting of halogen, cyano, thio, nitro, hydroxyl, carboxyl, (C₁-C₈)-alkyl, (C₁-C₈)-haloalkyl, (C₁-C₄)-alkoxy-(C₁-C₄)-alkyl, (C₂-C₈)-alkenyl, (C₂-C₈)-haloalkenyl, (C₂-C₈)-alkynyl, (C₂-C₈)-haloalkynyl, the 7 lastmentioned radicals only in the case of cyclic base radicals, (C₁-C₈)-alkoxy, (C₂-C₈)-alkenyloxy, (C₂-C₈)-alkynyloxy, (C₁-C₈)-haloalkoxy, (C₁-C₄)-alkoxy-(C₁-C₄)-alkoxy, (C₁-C₈)-alkylthio, (C₂-C₈)-alkenylthio, (C₂-C₈)-alkynylthio, radicals of the formulae —NR*R**, —CO—NR*R** and —O—CO—NR*R**,
      where each of the radicals R* and R** in the 3 lastmentioned formulae independently of the others is H, (C₁-C₈)-alkyl, (C₂-C₈)-alkenyl, (C₂-C₈)-alkynyl, benzyl, substituted benzyl, phenyl or substituted phenyl, or together with the nitrogen atom is a 3- to 8-membered heterocycle which, in addition to the nitrogen atom, may contain one or two further ring heteroatoms selected from the group consisting of N, O and S and which is unsubstituted or substituted by one or more radicals selected from the group consisting of (C₁-C₄)-alkyl and (C₁-C₄)-haloalkyl,
   and [(C₁-C₈)-alkoxy]carbonyl, [(C₁-C₈)-alkoxy]thiocarbonyl, [(C₂-C₈)-alkenyloxy]carbonyl, [(C₂-C₈)-alkynyloxy]carbonyl, [(C₁-C₈)-alkylthio]carbonyl, [(C₂-C₈)-alkenylthio]carbonyl, [(C₂-C₈)-alkynylthio]carbonyl, (C₁-C₈)-alkanoyl, [(C₂-C₈)-alkenyl]carbonyl, [(C₂-C₈)-alkynyl]carbonyl, (C₁-C₄)-alkylimino, (C₁-C₄)-alkoxyimino, [(C₁-C₈)-alkyl]carbonylamino, [(C₂-C₈)-alkenyl]carbonylamino, [(C₂-C₈)-alkynyl]carbonylamino, [(C₁-C₈)-alkoxy]carbonylamino, [(C₂-C₈)-alkenyloxy]carbonylamino, [(C₂-C₈)-alkynyloxy]carbonylamino, [(C₁-C₈)-alkylamino]carbonylamino, [(C₁-C₈)-alkyl]carbonyloxy, [(C₂-C₆)-alkenyl]carbonyloxy, [(C₂-C₆)-alkynyl]carbonyloxy, [(C₁-C₈)-alkoxy]carbonyloxy, [(C₂-C₈)-alkenyloxy]carbonyloxy, [(C₂-C₈)-alkynyloxy]carbonyloxy, (C₁-C₈)-alkylsulfinyl and (C₁-C₈)-alkylsulfonyl,
      where each of the 27 lastmentioned radicals is unsubstituted or substituted by one or more radicals selected from the group consisting of halogen, NO₂, (C₁-C₄)-alkoxy and optionally substituted phenyl,
   and phenyl, phenyl-(C₁-C₆)-alkoxy, phenyl-[(C₁-C₆)-alkoxy]carbonyl, phenoxy, phenoxy-(C₁-C₆)-alkoxy, phenoxy-[(C₁-C₆)-alkoxy]carbonyl, phenoxycarbonyl, phenylcarbonyloxy, phenoxycarbonyloxy, phenyl-[(C₁-C₆)-alkoxy]carbonyloxy, phenylcarbonylamino, phenyl-[(C₁-C₆)-alkyl]carbonylamino, phenyl-[(C₁-C₆)-alkyl]carbonyloxy, (C₃-C₇)-cycloalkyl, (C₃-C₇)-cycloalkoxy, (C₃-C₆)-cycloalkoxycarbonyl, (C₃-C₆)-cycloalkylcarbonyloxy, (C₃-C₆)-cycloalkoxycarbonyloxy, (C₃-C₆)-cycloalkyl-[(C₁-C₆)-alkyl]carbonyloxy and (C₃-C₆)-cycloalkyl-[(C₁-C₆)-alkoxy]carbonyloxy,
      where each of the 20 lastmentioned radicals is unsubstituted in the ring or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
   and radicals of the formulae —SiR′₃, —O—SiR′₃, (R′)₃Si—(C₁-C₆)-alkoxy, —CO—O—NR′₂, —O—N═CR′₂, —N═CR′₂, —O—NR′₂, —CH(OR′)₂ and —O—(CH₂)ₘ—CH(OR′)₂,
      in which each of the radicals R′ independently of the others is H, (C₁-C₄)-alkyl or phenyl which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro or substituted in two adjacent positions by a (C₂-C₆)-alkylene bridge, and m is an integer of from 0 to 6,
   and radicals of the formula R″O—CHR′″CH(OR″)—(C₁-C₆)-alkoxy, in which each of the radicals R″ independently of the others is H or (C₁-C₄)-alkyl or the radicals R″ together are a (C₁-C₆)-alkylene group and R′″ is H or (C₁-C₄)-alkyl,
   and also radicals of the formula Het¹, where Het¹ in each case independently of the others is a saturated, partially unsaturated or heteroaromatic heterocyclyl radical having 5 or 6 ring atoms, where the heterocyclic radical in question contains 1 to 3 ring heteroatoms selected from the group consisting of N, O and S and is optionally also fused, preferably benzo-fused, with a carbocyclic or heterocyclic ring, preferably a 5- or 6-membered ring having 0 or 1 to 3 ring heteroatoms selected from the group consisting of N, O and S, and is unsubstituted in the ring or in the polycyclic system or is substituted by one or more radicals selected from the group consisting of halogen, hydroxyl, (C₁-C₄)-alkyl, (C₁-C₄)-alkoxy, (C₁-C₄)-haloalkyl and (C₁-C₄)-haloalkoxy.

Here, more preference is also given to compounds (I) or salts thereof in which
R¹ is H, (C₁-C₁₂)-alkyl, (C₂-C₁₂)-alkenyl, (C₂-C₁₂)-alkynyl, (C₃-C₆)-cycloalkyl, (C₆-C₆)-cycloalkenyl, (C₅-C₆)-cycloalkynyl or phenyl,
   where each of the 7 lastmentioned radicals is unsubstituted or substituted by one or more radicals selected from the group consisting of halogen, cyano, thio, nitro, hydroxyl, carboxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, the 7 lastmentioned radicals only in the case of cyclic base radicals, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, radicals of the formulae —NR*R**, —CO—NR*R** and —O—CO—NR*R**, where each of the radicals R* and R** in the 3 lastmentioned formulae independently of the others is H, (C₁-C₄)-alkyl, (C₂-C₄)-alkenyl, (C₂-C₄)-alkynyl, benzyl, phenyl which is unsubstituted or substituted by one or more radicals selected from the group consisting of halogen, (C₁-C₄)-alkyl, (C₁-C₄)-haloalkyl and (C₁-C₄)-alkoxy, or together with the nitrogen atom is a piperidine, piperazine, pyrrolidine, pyrazolidine, piperazolidine or morpholine radical which is unsubstituted or substituted by one or more radicals selected from the group consisting of (C₁-C₄)-alkyl and (C₁-C₄)-haloalkyl,
   and [(C₁-C₄)-alkoxy]carbonyl, [(C₁-C₄)-alkyl]carbonylamino, [(C₁-C₄)-alkoxy]carbonylamino, [(C₁-C₄alkylamino]carbonylamino, [(C₁-C₄)-alkyl]carbonyloxy, [(C₁-C₄)-alkoxy]carbonyloxy and (C₁-C₄)-alkylsulfonyl,
      where each of the 7 lastmentioned radicals is unsubstituted or substituted by one or more radicals selected from the group consisting of halogen, NO₂, (C₁-C₄)-alkoxy and phenyl, which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
   and phenyl, phenyl-(C₁-C₄)-alkoxy, phenyl-[(C₁-C₄)-alkoxy]carbonyl, phenoxy, phenoxy-(C₁-C₄)-alkoxy, phenoxy-[(C₁-C₄)-alkoxy]carbonyl, phenoxycarbonyl, phenylcarbonyloxy, phenoxycarbonyloxy, phenyl-[(C₁-C₆)-alkoxy]carbonyloxy, phenylcarbonylamino, phenyl-[(C₁-C₄)-alkyl]carbonylamino, phenyl-[(C₁-C₄)-alkyl]carbonyloxy, (C₃-C₆)-cycloalkyl, (C₃-C₆)-cycloalkoxy, (C₃-C₆)-cycloalkoxycarbonyl, (C₃-C₆)-cycloalkylcarbonyloxy, (C₃-C₆)-cycloalkoxycarbonyloxy, (C₃-C₆)-cycloalkyl-[(C₁-C₆)-alkyl]carbonyloxy and (C₃-C₆)-cycloalkyl-[(C₁-C₆)-alkoxy]carbonyloxy,
      where each of the 20 lastmentioned radicals is unsubstituted in the ring or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
   and radicals of the formulae —CO—O—NR′₂, —O—N═CR′₂, —N═CR′₂, —O—NR′₂, —CH(OR′)₂ and —O—(CH₂)ₘ—CH(OR′)₂,
      in which each of the radicals R′ independently of the others is H, (C₁-C₄)-alkyl or phenyl which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro or is substituted in two adjacent positions by a (C₂-C₆)-alkylene bridge, and m is an integer of from 0 to 6,
   and radicals of the formula R″O—CHR′″CH(OR″)—(C₁-C₆)-alkoxy, in which each of the radicals R″ independently of the others is H or (C₁-C₄)-alkyl or the radicals R″ together are a (C₁-C₄)-alkylene group and R′″ is H or (C₁-C₂)-alkyl,
   and radicals of the formula Het¹, where Het¹ in each case independently of the others is a saturated, partially unsaturated or heteroaromatic heterocyclyl radical having 5 or 6 ring atoms, where the heterocyclic radical in question contains 1 to 3 ring heteroatoms selected from the group consisting of N, O and S and is optionally also fused, preferably benzo-fused, with a carbocyclic or heterocyclic ring, preferably a 5- or 6-membered ring having 0 or 1 to 3 ring heteroatoms selected from the group consisting of N, O and S, and is unsubstituted in the ring or in the polycyclic system or is substituted by one or more radicals selected from the group consisting of halogen, hydroxyl, (C₁-C₄)-alkyl, (C₁-C₄)-alkoxy, (C₁-C₄)-haloalkyl and (C₁-C₄)-haloalkoxy.

Here, more preference is also given to compounds (I) or salts thereof in which
R¹ is H, (C₁-C₁₂)-alkyl, (C₂-C₁₂)-alkenyl, (C₂-C₁₂)-alkynyl, (C₃-C₆)-cycloalkyl, (C₅-C₆)-cycloalkenyl, (C₅-C₆)-cycloalkynyl or phenyl,
   where each of the 7 lastmentioned radicals is unsubstituted or substituted by one or more radicals selected from the group consisting of halogen, cyano, thio, nitro, hydroxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, the 7 lastmentioned radicals only in the case of cyclic base radicals, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, radicals of the formulae —NR*R**, —CO—NR*R** and —O—CO—NR*R**,
      where each of the radicals R* and R** in the 3 lastmentioned formulae independently of the others is H, (C₁-C₄)-alkyl, (C₂-C₄)-alkenyl, (C₂-C₄)-alkynyl, benzyl, phenyl which is unsubstituted or substituted by one or more radicals selected from the group consisting of halogen, (C₁-C₄)-alkyl, (C₁-C₄)-haloalkyl and (C₁-C₄)-alkoxy, or together with the nitrogen atom is a piperidine, piperazine, pyrrolidine, pyrazolidine, piperazolidine or morpholine radical which is unsubstituted or substituted by one or more radicals selected from the group consisting of (C₁-C₄)-alkyl and (C₁-C₄)-haloalkyl,
   and [(C₁-C₄)-alkoxy]carbonyl, [(C₁-C₄)-alkyl]carbonylamino, [(C₁-C₄)-alkoxy]carbonylamino, [(C₁-C₄-alkylamino]carbonylamino, [(C₁-C₄)-alkyl]carbonyloxy, [(C₁-C₄)-alkoxy]carbonyloxy and (C₁-C₄)-alkylsulfonyl,
      where each of the 7 lastmentioned radicals is unsubstituted or substituted by one or more radicals selected from the group consisting of halogen, NO₂, (C₁-C₄)-alkoxy and phenyl, which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
   and phenyl, phenyl-(C₁-C₄)-alkoxy, phenyl-M-C₄)-alkoxy]carbonyl, phenoxy, phenoxy-(C₁-C₄)-alkoxy, phenoxy-[(C₁-C₄)-alkoxy]carbonyl, phenoxycarbonyl, phenylcarbonyloxy, phenylcarbonylamino, phenyl-[(C₁-C₄)-alkyl]carbonylamino, phenyl-[(C₁-C₄)-alkyl]carbonyloxy, (C₃-C₆)-cycloalkyl and (C₃-C₆)-cycloalkoxy,
      where each of the 13 lastmentioned radicals is unsubstituted in the ring or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
   and radicals of the formulae —CO—O—NR′₂, —O—N═CR′₂, —N═CR′₂, —O—NR′₂, —CH(OR′)₂ and —O—(CH₂)ₘ—CH(OR′)₂,
      in which each of the radicals R′ independently of the others is H, (C₁-C₄)-alkyl or phenyl which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro or is substituted in two adjacent positions by a (C₂-C₆)-alkylene bridge, and m is an integer of from 0 to 6,
   and radicals of the formula R″O—CHR′″CH(OR″)—(C₁-C₆)-alkoxy, in which each of the radicals R″ independently of the others is H or (C₁-C₄)-alkyl or the radicals R″ together are a (C₁-C₄)-alkylene group and R′″ is H or (C₁-C₂)-alkyl.

Here, more preference is also given to compounds (I) or salts thereof and their use in which
R¹ is H, (C₁-C₆)-alkyl, (C₂-C₆)-alkenyl, (C₂-C₆)-alkynyl or (C₃-C₆)-cycloalkyl, where each of the 4 lastmentioned radicals is unsubstituted or substituted by one or more radicals selected from the group consisting of halogen, (C₁-C₆)-alkyl, the latter substituent only in the case of cyclic base radicals, (C₁-C₄)-alkoxy, (C₁-C₄)-alkylthio, (C₃-C₆)-cycloalkyl which is unsubstituted or substituted by one or more radicals selected from the group consisting of halogen and (C₁-C₄)-alkyl, and phenyl which is unsubstituted or substituted by one or more radicals selected from the group consisting of halogen, (C₁-C₄)-alkyl, (C₁-C₄)-alkoxy and (C₁-C₄)-haloalkyl.

Here, particular preference is also given to compounds (I) or salts thereof in which
R¹ is H, (C₁-C₄)-alkyl, (C₂-C₄)-alkenyl or (C₂-C₄)-alkynyl, where each of the 3 lastmentioned radicals is unsubstituted or substituted by one or more radicals selected from the group consisting of halogen, (C₁-C₄)-alkoxy, (C₁-C₄)-alkylthio, cyclopropyl, cyclobutyl, where each of the two lastmentioned radicals is unsubstituted or substituted by one or more radicals selected from the group consisting of halogen and (C₁-C₄)-alkyl, and phenyl which is unsubstituted or substituted by one or more radicals selected from the group consisting of halogen, (C₁-C₄)-alkyl, (C₁-C₄)-alkoxy and (C₁-C₄)-haloalkyl.

More preferably,
R¹ is also a polycyclic radical based on (C₃-C₉)-cycloalkyl, (C₅-C₉)-cycloalkenyl, (C₅-C₉)-cycloalkynyl or phenyl, where the basic ring is fused, preferably benzo-fused, with a carbocyclic or heterocyclic ring, preferably a 5- or 6-membered ring having 0 or 1 to 3 ring heteroatoms selected from the group consisting of N, O and S, and where the basic ring or the polycyclic system is unsubstituted or substituted by one or more radicals selected from the group consisting of halogen, cyano, nitro, (C₁-C₆)-alkyl, (C₁-C₆)-haloalkyl, (C₁-C₄)-alkoxy-(C₁-C₄)alkyl, (C₂-C₄)-alkenyl, (C₂-C₄)-alkynyl, (C₁-C₄)-alkoxy, (C₁-C₄)-haloalkoxy, (C₁-C₄)-alkoxy-(C₁-C₄)alkoxy, (C₁-C₄)-alkylthio, (C₃-C₆)-cycloalkyl, (C₃-C₆)-cycloalkoxy, [(C₁-C₄)-alkoxy]carbonyl and [(C₁-C₄)-haloalkoxy]carbonyl.

Preference is also given to compounds (I) or salts thereof in which
R¹ is a saturated, partially unsaturated or heteroaromatic heterocyclyl radical having 3 to 9 ring atoms, preferably 5 or 6 ring atoms, which contains 1 to 4 heteroatoms, preferably 1 to 3 ring heteroatoms, selected from the group consisting of N, O and S, and which is unsubstituted or substituted by one or more radicals selected from the group consisting of halogen, cyano, thio, nitro, hydroxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, (C₃-C₆)-cycloalkyl, (C₃-C₆)-cycloalkoxy, [(C₁-C₈)-alkoxy]carbonyl, [(C₁-C₆)-haloalkoxy]carbonyl and oxo.

Preference is also given to compounds (I) or salts thereof in which
R¹ is a radical of the formula SiR^{a}R^{b}R^{c}, —NR^{a}R^{b} or —N═CR^{c}R^{d}, preferably of the formula —NR^{a}R^{b} or —N═CR^{c}R^{d},
   where in the 5 lastmentioned formulae each of the radicals R^{a}, R^{b}, R^{c} and R^{d} independently of the others is hydrogen, (C₁-C₄)-alkyl, (C₂-C₄)-alkenyl, (C₂-C₄)-alkynyl, benzyl, substituted benzyl, phenyl or substituted phenyl or R^{a} and R^{b} together with the nitrogen atom are a 3- to 8-membered heterocycle which in addition to the nitrogen atom may contain one or two further ring heteroatoms from the group consisting of N, O and S and which is unsubstituted or substituted by one or more radicals selected from the group consisting of (C₁-C₄)-alkyl and (C₁-C₄)-haloalkyl, or R^{c} and R^{d} together with the carbon atom are a 3- to 8-membered carbocyclic radical or heterocyclic radical which may contain 1 to 3 ring heteroatoms selected from the group consisting of N, O and S, where the carbocyclic or heterocyclic radical is unsubstituted or substituted by one or more radicals selected from the group consisting of (C₁-C₄)-alkyl and (C₁-C₄)-haloalkyl.

Particular preference is also given to compounds (I) or salts thereof in which
R¹ is H, methyl, ethyl, n-propyl, i-propyl, n-butyl, s-butyl, i-butyl, t-butyl, allyl, propargyl(prop-2-yn-1-yl), but-2-yn-1-yl, but-3-yn-1-yl, 2-chloroprop-2-en-1-yl, 3-phenylprop-2-yn-1-yl, 3,3-dichloroprop-2-en-1-yl, 3,3-dichloro-2-fluoroprop-2-en-1-yl, methylprop-2-yn-1-yl, 2-methylprop-2-en-1-yl, but-2-en-1-yl, but-3-en-1-yl, but-2-yn-1-yl, but-3-yn-1-yl, 4-chlorobut-2-yn-1-yl, 3-methylbut-2-en-1-yl, 3-methylbut-1-en-1-yl, (2E)-1-methylbut-2-en-1-yl, (E)-pent-3-en-2-yl or (Z)-pent-3-en-2-yl,
   phenyl, 2-carboxyphenyl, 2-chlorophenyl, 3-chlorophenyl, 4-chlorophenyl, 2-fluorophenyl, 3-fluorophenyl, 4-fluorophenyl, 3-methoxyphenyl, 4-methoxyphenyl, 2-methoxyphenyl, 3-methoxyphenyl, 4-methoxyphenyl, benzyl, 2-phenylethyl, 1-phenylethyl, (4-chlorophenyl)methyl [i.e. ═CH₂(4-Cl-Ph)], (4-fluorophenyl)methyl [i.e. ═CH₂(4-F-Ph)], (4-methoxyphenyl)methyl [i.e. ═CH₂(4-OMe-Ph)], 2-methoxyethyl, 2,2,2-trifluoroethyl, 1,1,1-trifluoroprop-2-yl, 2,2-difluoroethyl, 1,3-difluoroprop-2-yl, 2,3-dimethoxypropyl, 2,3-dimethoxyprop-2-yl, 2,2-dimethoxyeth-2-yl, 2-(2,2,2-trifluoroethoxy)ethyl, 2-fluoroethyl, 2-chloroethyl, 2-bromoethyl, 2-iodoethyl, 2,2,3,3,3-pentafluoropropyl, 1-hydroxyprop-2-yl, 2-hydroxyprop-2-yl, 2-hydroxyprop-1-yl, 3-hydroxypropyl, 3-hydroxyprop-2-yl,
   (2-methoxyethoxy)methyl; 2-(2-methoxyethoxy)ethyl; (2-ethoxyethoxy)methyl; 2-(2-ethoxyethoxy)ethyl,
   (acetoxy)methyl, (propanoyloxy)methyl, (2-methylpropanoyloxy)methyl, (2,2-dimethylpropanoyloxy)methyl, 1-(acetoxy)ethyl, 2-(acetoxy)ethyl, 2-(propanoyloxy)ethyl, 1-(propanoyloxy)ethyl, 1-(2-methylpropanoyloxy)eth-1-yl, 2-(2-methylpropanoyloxy)eth-1-yl, 2-(2,2-dimethylpropanoyloxy)ethyl [i.e. 1-(t-butylcarbonyloxy)ethyl], 2-(2,2-dimethyl propanoyloxy)ethyl;
   1-(2,2-dimethylpropanoyloxy)-2-methylprop-1-yl, 1-(t-butylcarbonyloxy)-2-methylprop-1-yl,
   (methoxycarbonyl)methyl, (ethoxycarbonyl)methyl, (n-propoxycarbonyl)methyl, (1-propoxycarbonyl)methyl, (n-butoxycarbonyl)methyl, (s-butoxycarbonyl)methyl, (1-butoxycarbonyl)methyl, (t-butoxycarbonyl)methyl, 1-(methoxycarbonyl)ethyl, 2-(methoxycarbonyl)ethyl, 1-(ethoxycarbonyl)ethyl, 2-(ethoxycarbonyl)ethyl, 1-(n-propoxycarbonyl)ethyl, 2-(n-propoxycarbonyl)ethyl, 1-(1-propoxycarbonyl)ethyl, 2-(1-propoxycarbonyl)ethyl, 1-(n-butoxycarbonyl)ethyl, 2-(n-butoxycarbonyl)ethyl, 1-(s-butoxycarbonyl)ethyl, 2-(s-butoxycarbonyl)ethyl, 1-(1-butoxycarbonyl)ethyl, 2-(1-butoxycarbonyl)ethyl, 1-(t-butoxycarbonyl)ethyl, 2-(t-butoxycarbonyl)ethyl,
   (methoxycarbonyloxy)methyl, (ethoxycarbonyloxy)methyl, (n-propoxycarbonyloxy)methyl, (1-propoxycarbonyloxy)methyl, (n-butoxycarbonyloxy)methyl, (s-butoxycarbonyloxy)methyl, (1-butoxycarbonyloxy)methyl, (t-butoxycarbonyloxy)methyl, 1-(methoxycarbonyloxy)ethyl, 2-(methoxycarbonyloxy)ethyl, 1-(ethoxycarbonyloxy)ethyl, 2-(ethoxycarbonyloxy)ethyl, 1-(n-propoxycarbonyloxy)ethyl, 2-(n-propoxycarbonyloxy)ethyl, 1-(1-propoxycarbonyloxy)ethyl, 2-(1-propoxycarbonyloxy)ethyl, 1-(n-butoxycarbonyloxy)ethyl, 2-(n-butoxycarbonyloxy)ethyl, 1-(s-butoxycarbonyloxy)ethyl, 2-(s-butoxycarbonyloxy)ethyl, 1-(1-butoxycarbonyloxy)ethyl, 2-(1-butoxycarbonyloxy)ethyl, 1-(t-butoxycarbonyloxy)ethyl, 2-(t-butoxycarbonyloxy)ethyl, (cyclohexoxycarbonyloxy)methyl, 1-(cyclohexoxycarbonyloxy)eth-1-yl, 2-(cyclohexoxycarbonyloxy)eth-1-yl,
   (acetyl)methyl, 1-(acetyl)ethyl, 2-(acetyl)ethyl, 1-(acetyl)propyl, 2-(acetyl)propyl, 3-(acetyl)propyl, (propanoyl)methyl, 1-(propanoyl)ethyl, 2-(propanoyl)ethyl, 1-(propanoyl)propyl, 2-(propanoyl)propyl, 3-(propanoyl)propyl, 1-(propanoyl)-2-methylpropyl,
   2-(ethylideneaminooxy)ethyl, 2-(prop-2-ylideneaminooxy)ethyl, 2-(but-2-ylideneaminooxy)ethyl, 2-(pent-3-ylideneaminooxy)ethyl,
   (N,N-dimethylamino)methyl, 2-(N,N-dimethylamino)eth-1-yl, 1-(N,N-dimethylamino)eth-1-yl, 2-(N,N-diethylamino)eth-1-yl, 1-(N,N-diethylamino)eth-1-yl, (N,N-diethylamino)methyl,
   (N,N-dimethylaminocarbonyl)methyl, 1-(N,N-dimethylaminocarbonyl)ethyl, 2-(N,N-dimethylaminocarbonyl)ethyl, (N,N-diethylaminocarbonyl)methyl, 1-(N,N-diethylaminocarbonyl)ethyl, 2-(N,N-diethylaminocarbonyl)ethyl,
   1-(dimethylamino)prop-2-yl [i.e. 2-(dimethylamino)-1-methylethyl], 1-(diethylamino)prop-2-yl,
   trimethylsilylmethyl, 1-(trimethylsilyl)ethyl, 2-(trimethylsilyl)ethyl,
   triethylsilylmethyl, 1-(triethylsilyl)ethyl, 2-(triethylsilyl)ethyl,
   cyclopropyl, cyclopropylmethyl, 1-cyclopropylethyl, 2-cyclopropylethyl,
   (1-methylcyclopropyl)methyl, 1-(1-methylcyclopropyl)ethyl, 2-(1-methylcyclopropyl)ethyl, (2,2-dichlorocyclopropyl)methyl, 1-(2,2-dichlorocyclopropyl)ethyl, 2-(2,2-dichlorocyclopropyl)ethyl, (2,2-dimethylcyclopropyl)methyl, 1-(2,2-dimethylcyclopropyl)ethyl, 2-(2,2-dimethylcyclopropyl)ethyl, cyclobutylmethyl, cyclopentylmethyl, cyclohexylmethyl or
   pyrid-2-yl, pyrid-3-yl, pyrid-4-yl, 2-chloropyrid-3-yl, 3-chloropyrid-2-yl,
   thien-2-yl, thien-3-yl, 2-chlorothien-3-yl, 3-chlorothien-2-yl, 4-chlorothien-2-yl, (1-ethyl-5-methyl-1H-pyrazol-4-yl)methyl, 1-(1-ethyl-5-methyl-1H-pyrazol-4-yl)ethyl, 2-(1-ethyl-5-methyl-1H-pyrazol-4-yl)ethyl,
   (1-ethyl-3-methyl-1H-pyrazol-4-yl)methyl, 1-(1-ethyl-3-methyl-1H-pyrazol-4-yl)ethyl, 2-(1-ethyl-3-methyl-1H-pyrazol-4-yl)ethyl,
   tetrahydrofuran-2-yl, tetrahydrofuran-3-yl, tetrahydrofuran-2-ylmethyl,
   tetrahydrofuran-3-ylmethyl, (5-methyl-2-oxo-1,3-dioxol-4-yl)methyl;
   oxetan-3-yl, (oxetan-3-yl)methyl, (oxetan-2-yl)methyl, (1,3-dioxolan-2-yl)methyl, (1,3-dioxolan-4-yl)methyl, 5-methyl-2-oxo-1,3-dioxolan-4-yl)methyl, (morpholin-4-yl)methyl; 1-(morpholin-4-yl)ethyl, 2-(morpholin-4-yl)ethyl, 2,3-dihydro-1H-inden-2-yl, dihydro-1H-inden-3-yl, dihydro-1H-inden-4-yl, dihydro-1H-inden-5-yl,
   1H-inden-2-yl, 1H-inden-3-yl, 1H-inden-4-yl, 1H-inden-5-yl, 1H-inden-6-yl or 1H-inden-7-yl.

Here, very particular preference is given to compounds (I) and salts thereof in which
R¹ is H, methyl, ethyl, n-propyl, i-propyl, phenyl, benzyl, CH₂(4-Cl-Ph), i.e. (4-chlorophenyl)methyl, CH₂(4-F-Ph), i.e. (4-fluorophenyl)methyl, CH₂(4-OMe-Ph), i.e. (4-methoxyphenyl)methyl, 2-methoxyethyl, tetrahydrofuran-2-ylmethyl, 2-(dimethylamino)ethyl, oxetan-3-yl, (3-methyloxetan-3-yl)methyl, 2,2,2-trifluoroethyl, 2,2-difluoroethyl, 2-fluoroethyl, 2,2,3,3,3-pentafluoropropyl, cyclopropylmethyl, 1-cyclopropylethyl, (1-methylcyclopropyl)methyl, (2,2-dichlorocyclopropyl)methyl, (2,2-dimethylcyclopropyl)methyl, allyl, propargyl (prop-2-yn-1-yl), 2-chloroprop-2-en-1-yl, 3-phenylprop-2-yn-1-yl, 3,3-dichloroprop-2-en-1-yl, 3,3-dichloro-2-fluoroprop-2-en-1-yl, methylprop-2-yn-1-yl, 2-methylprop-2-en-1-yl, but-2-en-1-yl, but-3-en-1-yl, but-2-yn-1-yl, but-3-yn-1-yl, 4-chlorobut-2-yn-1-yl, 3-methylbut-2-en-1-yl, 3-methylbut-1-en-1-yl, 1-(2E)-1-methylbut-2-en-1-yl, (E)-pent-3-en-2-yl or (Z)-pent-3-en-2-yl, cyclobutylmethyl, cyclopentylmethyl, cyclohexylmethyl or 1-ethyl-5-methyl-1H-pyrazol-4-ylmethyl, i.e. (1-ethyl-5-methyl-1H-pyrazol-4-yl)methyl.

Preference is also given to compounds of the formula (I) or salts thereof in which
R² is hydrogen, halogen or (C₁-C₄)-alkyl which is unsubstituted or substituted by one or more radicals selected from the group consisting of halogen, such as fluorine or chlorine, preferably hydrogen or (C₁-C₄)-alkyl, in particular hydrogen, methyl or ethyl, very particularly hydrogen or methyl.

Preference is also given to compounds of the formula (I) or salts thereof in which
R³ is hydrogen, halogen or (C₁-C₄)-alkyl which is unsubstituted or substituted by one or more radicals selected from the group consisting of halogen, such as fluorine or chlorine, preferably hydrogen or (C₁-C₄)-alkyl, in particular hydrogen or methyl, very particularly hydrogen.

Preference is also given to compounds of the formula (I) or salts thereof in which
R² is hydrogen and
R³ is hydrogen.

Preference is also given to the compounds of the formula (I) and salts thereof in which R² and R³ together with the carbon atom to which they are attached are (C₃-C₆)-cycloalkyl or (C₅-C₆)-cycloalkenyl, preferably (C₃-C₆)-cycloalkyl, where each of the 3 lastmentioned radicals is unsubstituted or substituted by one or more radicals selected from the group consisting of halogen and (C₁-C₄)-alkyl.

Here, R² and R³ are preferably together with the carbon atom to which they are attached cyclopropyl, cyclobutyl, cyclopentyl or cyclohexyl, in particular cyclopropyl, where each of the 4 lastmentioned radicals is unsubstituted or substituted by one or more radicals selected from the group consisting of halogen and methyl, preferably fluorine, chlorine and methyl.

Preference is also given to compounds of the formula (I) or salts thereof in which
R⁴ is hydrogen, halogen, cyano, (C₁-C₄)-alkyl, (C₂-C₄)-alkenyl or (C₂-C₄)-alkynyl,
   where each of the three lastmentioned radicals is unsubstituted or substituted by one or more radicals selected from the group consisting of halogen and hydroxyl, preferably unsubstituted or substituted by one or more radicals selected from the group consisting of halogen, such as fluorine and chlorine, or
   (C₃-C₆)-cycloalkyl which is unsubstituted or substituted by one or more radicals selected from the group consisting of halogen and (C₁-C₄)-alkyl, or
   phenyl which is unsubstituted or substituted by one or more radicals selected from the group consisting of halogen, nitro, (C₁-C₄)-alkyl, (C₁-C₄)-haloalkyl, (C₁-C₄)-alkoxy, (C₁-C₄)-haloalkoxy, (C₁-C₄)-alkoxy-(C₁-C₄)-alkoxy, (C₁-C₄)-alkylthio, [(C₁-C₄)-alkoxy]carbonyl and [(C₁-C₄)-haloalkoxy]carbonyl, or
   (C₁-C₄)-alkanoyl which is unsubstituted or substituted by one or more radicals selected from the group consisting of halogen, such as fluorine and chlorine, cyano, (C₁-C₄)-alkoxy, (C₁-C₄)-haloalkoxy and (C₁-C₂)-alkoxy-(C₁-C₂)-alkoxy, preferably formyl, or
   [(C₁-C₄)-alkoxy]carbonyl which is unsubstituted or substituted by one or more radicals selected from the group consisting of halogen, such as fluorine and chlorine, or
   [(C₃-C₆)-cycloalkoxy]carbonyl which is unsubstituted or substituted by one or more radicals selected from the group consisting of halogen and (C₁-C₄)-alkyl.

More preference is also given to compounds of the formula (I) or salts thereof in which
R⁴ is hydrogen, halogen, such as fluorine or chlorine, cyano, (C₁-C₄)-alkyl which is optionally substituted by hydroxy[=(C₁-C₄)-hydroxyalkyl], (C₁-C₄)-haloalkyl, cyclopropyl or cyclobutyl, where each of the two lastmentioned radicals is unsubstituted or substituted by one or more radicals selected from the group consisting of halogen, such as fluorine and chlorine, and (C₁-C₄)-alkyl, or
   phenyl which is unsubstituted or substituted by one or more radicals selected from the group consisting of halogen, (C₁-C₄)-alkyl, (C₁-C₄)-haloalkyl, (C₁-C₄)-alkoxy and (C₁-C₄)-alkylthio, or
   (C₁-C₄)-alkanoyl which is unsubstituted or substituted by one or more radicals selected from the group consisting of halogen, such as fluorine and chlorine, preferably formyl, or
   [(C₁-C₄)-alkoxy]carbonyl or [(C₁-C₄)-haloalkoxy]carbonyl,
      preferably
R⁴ is hydrogen, halogen, such as fluorine or chlorine, cyano, methyl, ethyl, n-propyl, i-propyl, CH₂Cl, CHCl₂, CCl₃, CH₂F, CHF₂, CF₃ or formyl.

More preferably
R⁴ is hydrogen, fluorine, chlorine, methyl, ethyl, n-propyl, i-propyl, CH₂Cl, CHCl₂, CCl₃, CH₂F, CHF₂ or CF₃, in particular methyl.

Preference is also given to compounds of the formula (I) or salts thereof in which
R⁵ is phenyl which is unsubstituted or preferably substituted and including substituents has 6 to 24 carbon atoms, in particular 6 to 20 carbon atoms, or
   a 5- or 6-membered heteroaromatic radical having 1 to 3 ring heteroatoms from the group consisting of N, O and S which is unsubstituted or substituted and including substituents has 1 to 24 carbon atoms, in particular 1 to 20 carbon atoms.

More preference is also given to the compounds of the formula (I) or salts thereof and their use in which
R⁵ is a phenyl radical or a 5- or 6-membered heteroaromatic radical having 1 to 3 ring heteroatoms from the group consisting of N, O and S, where the phenyl radical or the heterocyclic radical is unsubstituted or substituted by one or more radicals selected from the group consisting of the radicals
   (a) halogen, hydroxyl, amino, nitro, carboxyl, cyano and carbamoyl,
   (b) (C₁-C₆)-alkyl, (C₂-C₆)-alkenyl, (C₂-C₆)-alkynyl, (C₁-C₆)-alkoxy, (C₁-C₆)-alkenyloxy and (C₁-C₆)-alkynyloxy, where each of the 6 lastmentioned radicals is unsubstituted or substituted by one or more radicals selected from the group consisting of halogen, (C₁-C₄)-alkoxy, (C₁-C₆)-haloalkoxy, (C₁-C₄)-alkylthio, mono- and di-[(C₁-C₄)-alkyl]amino, hydroxyl, carboxyl, [(C₁-C₄)-alkoxy]carbonyl, [(C₁-C₄)-haloalkoxy]carbonyl, mono- and di-[(C₁-C₄)-alkyl]aminocarbonyl and cyano,
   (c) (C₁-C₆)-alkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- and di-[(C₁-C₄)-alkyl]aminocarbonyl, mono- and di-[(C₁-C₆)acyl]amino, mono- and di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₁-C₆)-alkylsulfinyloxy, (C₁-C₆)-haloalkylsulfinyloxy, (C₁-C₆)-alkylsulfonyloxy, (C₁-C₆)-haloalkylsulfonyloxy, (C₁-C₆)-alkylsulfato, (C₁-C₆)-haloalkylsulfato and
   (d) (C₃-C₆)-cycloalkyl, (C₃-C₆)-cycloalkyloxy, phenyl and phenoxy,
      where each of the 4 lastmentioned radicals is unsubstituted or substituted by one or more radicals selected from the group consisting of halogen, (C₁-C₄)-alkyl, (C₁-C₄)-haloalkyl, (C₁-C₄)-alkoxy, (C₁-C₆)-haloalkoxy and (C₁-C₄)-alkylthio,
and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen, (C₁-C₄)-alkyl, (C₁-C₄)-haloalkyl, (C₁-C₄)-alkoxy, (C₁-C₆)-haloalkoxy and (C₁-C₄)-alkylthio.

Here, more preferably
R⁵ is phenyl
   which is unsubstituted or preferably substituted by one or more radicals selected from the group consisting of the radicals
      (a) halogen, hydroxyl, amino, nitro, carboxyl, cyano and carbamoyl,
      (b) (C₁-C₆)-alkyl, (C₂-C₆)-alkenyl, (C₂-C₆)-alkynyl and (C₁-C₆)-alkoxy, where each of the 4 lastmentioned radicals is unsubstituted or substituted by one or more radicals selected from the group consisting of halogen, (C₁-C₄)-alkoxy, (C₁-C₆)-haloalkoxy, (C₁-C₄)-alkylthio, hydroxyl, carboxyl, [(C₁-C₄)-alkoxy]carbonyl and cyano,
      (c) (C₁-C₆)-alkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- and di-[(C₁-C₄)-alkyl]aminocarbonyl, mono- and di-[(C₁-C₆)acyl]amino, mono- and di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₁-C₆)-alkylsulfinyloxy, (C₁-C₆)-alkylsulfonyloxy and (C₁-C₆)-haloalkylsulfonyloxy and
      (d) (C₃-C₆)-cycloalkyl, (C₃-C₆)-cycloalkyloxy, phenyl and phenoxy, where each of the 4 lastmentioned radicals is unsubstituted or substituted by one or more radicals selected from the group consisting of halogen, (C₁-C₄)-alkyl, (C₁-C₄)-haloalkyl, (C₁-C₄)-alkoxy, (C₁-C₆)-haloalkoxy and (C₁-C₄)-alkylthio,
   and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen, (C₁-C₄)-alkyl, (C₁-C₄)-haloalkyl, (C₁-C₄)-alkoxy, (C₁-C₆)-haloalkoxy and (C₁-C₄)-alkylthio,
      or
R⁵ is a 5- or 6-membered heteroaromatic radical having 1 to 3 ring heteroatoms from the group consisting of N, O and S which is unsubstituted or substituted by one or more radicals selected from the group consisting of the radicals
   (a) halogen, hydroxyl, amino, nitro, carboxyl, cyano and carbamoyl,
   (b) (C₁-C₆)-alkyl, (C₂-C₆)-alkenyl, (C₂-C₆)-alkynyl and (C₁-C₆)-alkoxy, where each of the 6 lastmentioned radicals is unsubstituted or substituted by one or more radicals selected from the group consisting of halogen, (C₁-C₄)-alkoxy, (C₁-C₆)-haloalkoxy, (C₁-C₄)-alkylthio, mono- and di-[(C₁-C₄)-alkyl]amino, hydroxyl, carboxyl, [(C₁-C₄)-alkoxy]carbonyl and cyano,
   (c) (C₁-C₆)-alkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- and di-[(C₁-C₄)-alkyl]aminocarbonyl, mono- and di-[(C₁-C₆)acyl]amino, mono- and di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₁-C₆)-alkylsulfinyloxy, (C₁-C₆)-alkylsulfonyloxy and (C₁-C₆)-haloalkylsulfonyloxy and
   (d) (C₃-C₆)-cycloalkyl, (C₃-C₆)-cycloalkyloxy, phenyl and phenoxy, where each of the 4 lastmentioned radicals is unsubstituted or substituted by one or more radicals selected from the group consisting of halogen, (C₁-C₄)-alkyl, (C₁-C₄)-haloalkyl, (C₁-C₄)-alkoxy, (C₁-C₆)-haloalkoxy and (C₁-C₄)-alkylthio,
   and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen, (C₁-C₄)-alkyl, (C₁-C₄)-haloalkyl, (C₁-C₄)-alkoxy, (C₁-C₆)-haloalkoxy and (C₁-C₄)-alkylthio.

More preference is also given to the compounds of the formula (I) or salts thereof and their use in which
R⁵ is phenyl
   which is unsubstituted or preferably substituted by one or more radicals selected from the group consisting of halogen, hydroxyl, amino, nitro, carboxyl, cyano, carbamoyl, (C₁-C₆)-alkyl, (C₂-C₄)-alkenyl, (C₂-C₄)-alkynyl, (C₁-C₆)-haloalkyl, (C₁-C₄)-alkoxy-(C₁-C₄)-alkyl, (C₁-C₄)-alkylthio-(C₁-C₄)-alkyl, mono- and di-[(C₁-C₄)-alkyl]amino-(C₁-C₄)-alkyl, hydroxy-(C₁-C₄)-alkyl, carboxy-(C₁-C₄)-alkyl, cyano-(C₁-C₄)-alkyl, (C₁-C₆)-alkoxy, which may optionally also be halogenated[=(C₁-C₆)-haloalkoxy)], (C₁-C₄)-alkoxy-(C₁-C₄)-alkoxy, (C₁-C₆)-alkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- and di-[(C₁-C₄)-alkyl]aminocarbonyl, mono- and di-[(C₁-C₆)acyl]amino, mono- and di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₁-C₄)-alkylsulfonyloxy, (C₃-C₆)-cycloalkyl, (C₃-C₆)-cycloalkyloxy, phenyl and phenoxy,
      where each of the 4 lastmentioned radicals is unsubstituted or substituted by one or more radicals selected from the group consisting of halogen, (C₁-C₄)-alkyl and (C₁-C₄)-haloalkyl,
   and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen and (C₁-C₆)-alkyl,
   or
R⁵ is a 5- or 6-membered heteroaromatic radical having 1 to 3 ring heteroatoms selected from the group consisting of N, O and S,
   which is unsubstituted or substituted by one or more radicals selected from the group consisting of halogen, hydroxyl, amino, nitro, carboxyl, cyano, carbamoyl, (C₁-C₆)-alkyl, (C₂-C₄)-alkenyl, (C₂-C₄)-alkynyl, (C₁-C₆)-haloalkyl, (C₁-C₄)-alkoxy-(C₁-C₄)-alkyl, (C₁-C₄)-alkylthio-(C₁-C₄)-alkyl, mono- and di-[(C₁-C₄)-alkyl]amino-(C₁-C₄)-alkyl, hydroxy-(C₁-C₄)-alkyl, carboxy-(C₁-C₄)-alkyl, cyano-(C₁-C₄)-alkyl, (C₁-C₆)-alkoxy, which may optionally also be halogenated[=(C₁-C₆)-haloalkoxy)], (C₁-C₄)-alkoxy-(C₁-C₄)-alkoxy, (C₁-C₆)-alkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- and di-[(C₁-C₄)-alkyl]aminocarbonyl, mono- and di-[(C₁-C₆)acyl]amino, mono- and di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₁-C₄)-alkylsulfonyloxy, (C₃-C₆)-cycloalkyl, (C₃-C₆)-cycloalkyloxy, phenyl and phenoxy,
      where each of the 4 lastmentioned radicals is unsubstituted or substituted by one or more radicals selected from the group consisting of halogen, (C₁-C₄)-alkyl and (C₁-C₄)-haloalkyl,
   and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen and (C₁-C₆)-alkyl.

Here, more preference is also given to compounds of the formula (I) or salts thereof in which
R⁵ is phenyl which is unsubstituted or preferably substituted by one or more radicals selected from the group consisting of halogen, hydroxyl, nitro, carboxyl, cyano, (C₁-C₄)-alkyl, (C₁-C₄)-haloalkyl, (C₁-C₄)-alkoxy-(C₁-C₄)-alkyl, (C₁-C₄)-alkylthio-(C₁-C₄)-alkyl, hydroxy-(C₁-C₄)-alkyl, (C₁-C₄)-alkoxy, which may optionally also be halogenated[═(C₁-C₄)-haloalkoxy], (C₁-C₄)-alkoxy-(C₁-C₄)-alkoxy, (C₁-C₄)-alkylthio, [(C₁-C₄)-alkoxy]carbonyl, [(C₁-C₆)-haloalkoxy]carbonyl, (C₁-C₄)-alkylsulfinyl, (C₁-C₄)-haloalkylsulfinyl, (C₁-C₄)-alkylsulfonyl, (C₁-C₄)-haloalkylsulfonyl, (C₃-C₆)-cycloalkyl, (C₃-C₆)-cycloalkyloxy, phenyl and phenoxy,
   where each of the 4 lastmentioned radicals is unsubstituted or substituted by one or more radicals selected from the group consisting of halogen, (C₁-C₄)-alkyl and (C₁-C₄)-haloalkyl,
   and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen and (C₁-C₄)-alkyl,
   or
R⁵ is a 5- or 6-membered heteroaromatic radical having 1 to 3 ring heteroatoms selected from the group consisting of N, O and S,
   which is unsubstituted or substituted by one or more radicals selected from the group consisting of halogen, hydroxyl, carboxyl, cyano, amino, (C₁-C₄)-alkyl, (C₂-C₄)-alkenyl, (C₂-C₄)-alkynyl, (C₁-C₄)-haloalkyl, (C₁-C₄)-alkoxy-(C₁-C₄)-alkyl, (C₁-C₄)-alkylthio-(C₁-C₄)-alkyl, hydroxy-(C₁-C₄)-alkyl, (C₁-C₄)-alkoxy, which may optionally also be halogenated [═(C₁-C₄)-haloalkoxy], (C₁-C₄)-alkoxy-(C₁-C₄)-alkoxy, (C₁-C₄)-alkylthio, [(C₁-C₆)-alkoxy]carbonyl, [(C₁-C₆)-haloalkoxy]carbonyl, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, mono- and di-[(C₁-C₄)-alkyl]amino, phenyl and (C₃-C₆)-cycloalkyl which is unsubstituted or substituted by one or more radicals selected from the group consisting of halogen, (C₁-C₄)-alkyl and (C₁-C₄)-haloalkyl,
   and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen and (C₁-C₄)-alkyl.

Here, even more preference is also given to compounds of the formula (I) or salts thereof in which
R⁵ is phenyl which is unsubstituted or preferably substituted by one or more radicals selected from the group consisting of halogen, cyano, (C₁-C₄)-alkyl, (C₁-C₄)-haloalkyl, (C₁-C₄)-alkoxy, (C₁-C₄)-alkylthio, (C₂-C₄)-alkenyl, (C₂-C₄)-alkynyl, (C₃-C₆)-cycloalkyl, phenyl, amino, mono- and di-[(C₁-C₄)-alkyl]amino and (C₁-C₄)-alkylsulfonyloxy and is optionally benzo-fused,
   or
R⁵ is a 5- or 6-membered heteroaromatic radical having 1 to 3 ring heteroatoms selected from the group consisting of N, O and S,
   which is unsubstituted or substituted by one or more radicals selected from the group consisting of halogen, hydroxyl, (C₁-C₄)-alkyl, (C₁-C₄)-haloalkyl, (C₁-C₄)-alkoxy, (C₁-C₄)-alkylthio, (C₂-C₄)-alkenyl, (C₂-C₄)-alkynyl, (C₃-C₆)-cycloalkyl, phenyl, amino, mono- and di-[(C₁-C₄)-alkyl]amino and (C₁-C₄)-alkylsulfonyloxy and is optionally benzo-fused.

Here, even more preference is also given to compounds of the formula (I) or salts thereof in which
R⁵ is phenyl which is unsubstituted or preferably substituted by one or more radicals selected from the group consisting of halogen, cyano, (C₁-C₄)-alkyl, (C₁-C₄)-haloalkyl, (C₁-C₄)-alkoxy and (C₁-C₄)-alkylthio and is optionally benzo-fused,
   or
R⁵ is a 5- or 6-membered heteroaromatic radical having 1 to 3 ring heteroatoms selected from the group consisting of N, O and S,
   which is unsubstituted or substituted by one or more radicals selected from the group consisting of halogen, hydroxyl, (C₁-C₄)-alkyl, (C₁-C₄)-haloalkyl, (C₁-C₄)-alkoxy and (C₁-C₄)-alkylthio and is optionally benzo-fused.

Here, particular preference is given to compounds of the formula (I) or salts thereof in which
R⁵ is phenyl which is unsubstituted or preferably substituted by one or more radicals selected from the group consisting of halogen, such as fluorine, chlorine, bromine and iodine, cyano, methyl, ethyl, n-propyl, i-propyl, n-butyl, sec-butyl, i-butyl, t-butyl, trifluoromethyl, trichloromethyl, methoxy and ethoxy and is optionally benzo-fused,
   preferably
R⁵ is phenyl, 2-fluorophenyl, 2-chlorophenyl, 2-bromophenyl, 2-iodophenyl, 2-methylphenyl, 4-(tert-butyl)phenyl, 2-trifluoromethylphenyl, 2-methoxyphenyl, 2-cyanophenyl, 2-nitrophenyl, 4-nitrophenyl, 3-fluorophenyl, 3-chlorophenyl, 3-bromophenyl, 3-iodophenyl, 3-methylphenyl, 3-trifluoromethylphenyl, 3-methoxyphenyl, 4-carboxyphenyl, 4-fluorophenyl, 4-chlorophenyl, 4-bromophenyl, 4-iodophenyl, 4-methylphenyl, 4-trifluoromethylphenyl, 4-methoxyphenyl, 2,3-dichlorophenyl, 2,3-dimethylphenyl, 2,4-dichlorophenyl, 2,4-dimethylphenyl, 2,5-dichlorophenyl, 2,5-dimethylphenyl, 2,6-dichlorophenyl, 2,4-difluorophenyl, 2,6-dimethylphenyl, 3,4-dichlorophenyl, 3,4-difluorophenyl, 2,6-dimethylphenyl, 3,4-dichlorophenyl, 3,4-dimethylphenyl, 3,5-dichlorophenyl, 3,5-dimethylphenyl, 2-chloro-3-methylphenyl, 2-chloro-4-methylphenyl, 2-chloro-5-methylphenyl, 2-chloro-6-methylphenyl, 3-chloro-4-methylphenyl, 3-chloro-5-methylphenyl, 3-chloro-2-methylphenyl, 4-chloro-2-methylphenyl, 4-chloro-3-methylphenyl, 4-bromo-3-methylphenyl, 5-chloro-2-methylphenyl, 4-phenylphenyl, 3-trifluoromethyl-4-chlorophenyl, 4-phenoxyphenyl, 4-carboxymethylphenyl, 4-acetylphenyl (=4-methylcarbonylphenyl) or 1,3-benzodioxol-5-yl.

Here, preference is also given to compounds of the formula (I) or salts thereof in which
R⁵ is 2-pyridyl, 3-pyridyl, 4-pyridyl, 2-thienyl, 3-thienyl, 2-furyl, 3-furyl, 2-pyrrolyl, 3-pyrrolyl, 2-pyrimidinyl, 4-pyrimidinyl, 5-pyrimidinyl, 3-pyridazinyl, 4-pyridazinyl, 2-pyrazinyl, 3-pyrazinyl, 2-imidazolinyl, 4-imidazolinyl, 1-pyrazolyl, 3-pyrazolyl, 4-pyrazolyl, 5-pyrazolyl, 1,3,5-triazin-2-yl, 1,2,4-triazin-3-yl, 1,2,4-triazin-5-yl, 2-thiazolyl, 1,3-benzothiazol-2-yl, 4-thiazolyl, 5-thiazolyl, 3-isothiazolyl, 4-isothiazolyl, 5-isothiazolyl, 2-oxazolyl, 4-oxazolyl, 5-oxazolyl, 3-isoxazolyl, 4-isoxazolyl, 5-isoxazolyl, thiadiazolyl or triazolyl, 3-isoquinolinyl, 2-quinolinyl, 1,3-benzthiazol-2-yl, 1,3-benzoxazol-2-yl, preferably 2-pyridyl, 3-pyridyl, 4-pyridyl, 2-pyrimidinyl, 2-pyrazinyl, 2-thienyl, 3-thienyl, 2-furyl or 2-thiazolyl, 3-isoquinolinyl, 2-quinolinyl, where each of the heteroaromatic radicals mentioned above is unsubstituted or substituted, preferably substituted by the radicals already mentioned above as being preferred, in particular by one or more radicals selected from the group consisting of halogen, hydroxyl, (C₁-C₄)-alkyl, (C₁-C₄)-haloalkyl, (C₁-C₄)-alkoxy, (C₁-C₄)-alkylthio, (C₂-C₄)-alkenyl, (C₂-C₄)-alkynyl, (C₁-C₄)-alkylamino, di-[(C₁-C₄)-alkyl]amino and (C₁-C₄)-alkylsulfonyloxy.

Here, preference is also given to compounds of the formula (I) or salts thereof in which

R⁵ is 2-pyridyl, 3-pyridyl, 4-pyridyl, 2-thienyl, 3-thienyl, 2-furyl, 3-furyl, 2-pyrrolyl, 3-pyrrolyl, 2-pyrimidinyl, 4-pyrimidinyl, 5-pyrimidinyl, 3-pyridazinyl, 4-pyridazinyl, 2-pyrazinyl, 3-pyrazinyl, 2-imidazolinyl, 4-imidazolinyl, 1-pyrazolyl, 3-pyrazolyl, 4-pyrazolyl, 5-pyrazolyl, 1,3,5-triazin-2-yl, 1,2,4-triazin-3-yl, 1,2,4-triazin-5-yl, 2-thiazolyl, 2-(1,3-benzothiazolyl), isoquinolin-3-yl, quinolin-2-yl, 4-thiazolyl, 5-thiazolyl, 3-isothiazolyl, 4-isothiazolyl, 5-isothiazolyl, 2-oxazolyl, 4-oxazolyl, 5-oxazolyl, 3-isoxazolyl, 4-isoxazolyl, 5-isoxazolyl, thiadiazolyl or triazolyl, preferably 2-pyridyl, 3-pyridyl, 4-pyridyl, 2-pyrimidinyl, 2-pyrazinyl, 2-thienyl, 3-thienyl, 2-furyl or 2-thiazolyl, where each of the heteroaromatic radicals mentioned above is unsubstituted or substituted, preferably by the radicals already mentioned above as being preferred (i.e. radicals mentioned as substituents), more preferably by one or more radicals selected from the group consisting of halogen, hydroxyl, (C₁-C₄)-alkyl, (C₁-C₄)-haloalkyl, (C₁-C₄)-alkoxy and (C₁-C₄)-alkylthio and also (C₂-C₄)-alkenyl, (C₂-C₄)-alkynyl, (C₁-C₄)-alkylamino, di-[(C₁-C₄)-alkyl]amino and (C₁-C₄)-alkylsulfonyloxy, in particular substituted by one or more radicals selected from the group consisting of halogen, hydroxyl, (C₁-C₄)-alkyl, (C₁-C₄)-haloalkyl, (C₁-C₄)-alkoxy, (C₁-C₄)-alkylthio.

Here, particular preference is also given to compounds of the formula (I) or salts thereof in which
R⁵ is 2-pyridyl, 3-fluoropyrid-2-yl, 3-chloropyrid-2-yl, 3-bromopyrid-2-yl, 3-methylpyrid-2-yl, 3-methoxypyrid-2-yl, 3-trifluoromethylpyrid-2-yl, 4-fluoropyrid-2-yl, 4-chloropyrid-2-yl, 4-bromopyrid-2-yl, 4-methylpyrid-2-yl, 4-methoxypyrid-2-yl, 4-trifluoromethylpyrid-2-yl, 5-fluoropyrid-2-yl, 5-chloropyrid-2-yl, 5-bromopyrid-2-yl, 5-methylpyrid-2-yl, 5-methoxypyrid-2-yl, 5-trifluoromethylpyrid-2-yl, 6-fluoropyrid-2-yl, 6-chloropyrid-2-yl, 6-bromopyrid-2-yl, 6-methylpyrid-2-yl, 6-methoxypyrid-2-yl, 6-trifluoromethylpyrid-2-yl, 4,6-dimethylpyridin-2-yl, 4-chloro-6-methylpyrid-2-yl, 4-methyl-5-chloropyrid-2-yl
   or
   3-pyridyl, 2-fluoropyrid-3-yl, 2-chloropyrid-3-yl, 2-bromopyrid-3-yl, 2-methylpyrid-3-yl, 2-methoxypyrid-3-yl, 2-trifluoromethylpyrid-3-yl, 4-fluoropyrid-3-yl, 4-chloropyrid-3-yl, 4-bromopyrid-3-yl, 4-methylpyrid-3-yl, 4-methoxypyrid-3-yl, 4-trifluoromethylpyrid-3-yl, 5-fluoropyrid-3-yl, 5-chloropyrid-3-yl, 5-bromopyrid-3-yl, 5-methylpyrid-3-yl, 5-methoxypyrid-3-yl, 5-trifluoromethylpyrid-3-yl, 6-fluoropyrid-3-yl, 6-chloropyrid-3-yl, 6-bromopyrid-3-yl, 6-methylpyrid-3-yl, 6-methoxypyrid-3-yl, 6-trifluoromethylpyrid-3-yl, 6-hydroxypyridin-3-yl,
   4-pyridyl, 2-fluoropyrid-4-yl, 2-chloropyrid-4-yl, 2-bromopyrid-4-yl, 2-methylpyrid-4-yl, 2-methoxypyrid-4-yl, 2-trifluoromethylpyrid-4-yl, 3-fluoropyrid-4-yl, 3-chloropyrid-4-yl, 3-bromopyrid-4-yl, 3-methylpyrid-3-yl, 3-methoxypyrid-4-yl, 3-trifluoromethylpyrid-4-yl, 5-iodopyrid-2-yl, 5-dimethylaminopyrid-2-yl, 5-methylaminopyrid-2-yl, 5-methylthiopyrid-2-yl, 5-difluoromethoxypyrid-2-yl, 5-hydroxypyrid-2-yl, 5-ethynylpyrid-2-yl, 5-cyclopropylpyrid-2-yl, 5-allylpyrid-2-yl, 5-phenylpyrid-2-yl, 5-aminopyrid-2-yl or 5-methylsulfonyloxypyrid-2-yl or
   2-thienyl, 3-fluorothien-2-yl, 3-chlorothien-2-yl, 3-bromothien-2-yl, 3-methylthien-2-yl, 3-methoxythien-2-yl, 3-trifluoromethylthien-2-yl, 4-fluorothien-2-yl, 4-chlorothien-2-yl, 4-bromothien-2-yl, 4-methylthien-2-yl, 4-methoxythien-2-yl, 4-trifluoromethylthien-2-yl, 5-fluorothien-2-yl, 5-chlorothien-2-yl, 5-bromothien-2-yl, 5-iodo-2-thienyl, 5-methylthien-2-yl, 5-methoxythien-2-yl, 5-trifluoromethylthien-2-yl or
   3-thienyl, 2-fluorothien-3-yl, 2-chlorothien-3-yl, 2-bromothien-3-yl, 2-methylthien-3-yl, 2-methoxythien-3-yl, 2-trifluoromethylthien-3-yl, 4-fluorothien-3-yl, 4-chlorothien-3-yl, 4-bromothien-3-yl, 4-methylthien-3-yl, 4-methoxythien-3-yl, 4-trifluoromethylthien-3-yl, 5-fluorothien-3-yl, 5-chlorothien-3-yl, 5-bromothien-3-yl, 5-methylthien-3-yl, 5-methoxythien-3-yl, 5-trifluoromethylthien-3-yl or
   2-furyl, 3-fluorofur-2-yl, 3-chlorofur-2-yl, 3-bromofur-2-yl, 3-methylfur-2-yl, 3-methoxyfur-2-yl, 3-trifluoromethylfur-2-yl, 4-fluorofur-2-yl, 4-chlorofur-2-yl, 4-bromofur-2-yl, 4-methylfur-2-yl, 4-methoxyfur-2-yl, 4-trifluoromethylfur-2-yl, 5-fluorofur-2-yl, 5-chlorofur-2-yl, 5-bromofur-2-yl, 5-methylfur-2-yl, 5-methoxyfur-2-yl or 5-trifluoromethylfur-2-yl or
   2-thiazolyl, 4-methylthiazol-2-yl, 5-methylthiazol-2-yl, 5-bromothiazol-2-yl, 5-chlorothiazol-2-yl, 4,5-dimethylthiazol-2-yl, 4,5-dichlorothiazol-2-yl,
   2-(1,3-benzothiazolyl), 7-chloro-(1,3-benzothiazol-2-yl), isoquinolin-3-yl, quinolin-2-yl or
   1,3-benzothiazol-2-yl, 7-chloro-1,3-benzothiazol-2-yl, 2-bromothiazol-4-yl, 2-chlorothiazol-4-yl, 4-thiazolyl, 2-methylthiazol-4-yl, thiazol-5-yl, 2-methylthiazol-5-yl, 6-chloro-1,3-benzothiazol-2-yl, 7-bromo-1,3-benzothiazol-2-yl, 6-bromo-1,3-benzothiazol-2-yl, 1,3-benzoxazol-2-yl, 7-chloro-1,3-benzoxazol-2-yl, 6-chloro-1,3-benzoxazol-2-yl, 7-bromo-1,3-benzoxazol-2-yl, 6-bromo-1,3-benzoxazol-2-yl or
   2-pyrazinyl, 5-methylpyrazin-2-yl,
   1,5-dimethylpyrazol-3-yl, 1-methylpyrazol-3-yl or 1-methylpyrazol-5-yl or 2-pyrimidinyl, 5-F-pyrimidin-2-yl, 5-chloropyrimidin-2-yl, 5-bromopyrimidin-2-yl, 5-methylpyrimidin-2-yl, 4,6-dimethylpyrimidin-2-yl, 5-fluoropyrimidin-2-yl, 5-iodopyrimidin-2-yl or
   3-pyridazinyl, 6-methylpyridazin-3-yl,
   3-(1,2,4)-triazinyl, i.e. 1,2,4-triazin-3-yl, or 6-methyl-(1,2,4)-triazin-3-yl, i.e. 6-methyl-1,2,4-triazin-3-yl,
      preferably
   R⁵ is 2-pyridyl, 5-fluoropyrid-2-yl, 5-chloropyrid-2-yl, 5-bromopyrid-2-yl, 5-methylpyrid-2-yl, 5-methoxypyrid-2-yl, 5-trifluoromethylpyrid-2-yl, 3-pyridyl, 6-fluoropyrid-3-yl, 6-chloropyrid-3-yl, 6-bromopyrid-3-yl, 6-methylpyrid-3-yl, 6-methoxypyrid-3-yl or 6-trifluoromethylpyrid-3-yl, 4,6-Me₂-pyridin-2-yl, 2-thienyl, 3-chlorothien-2-yl, 3-methylthien-2-yl, 4-chlorothien-2-yl, 4-methylthien-2-yl, 5-chlorothien-2-yl, 5-bromothien-2-yl, 5-iodo-2-thienyl, 5-methylthien-2-yl, 2-thiazolyl, 5-bromothiazol-2-yl, 5-chlorothiazol-2-yl, 4,5-dimethylthiazol-2-yl, 4,5-dichlorothiazol-2-yl, 2-(1,3-benzothiazolyl), isoquinolin-3-yl (=3-isoquinolinyl), quinolin-2-yl (=2-quinolinyl), 2-pyrazinyl, 5-methylpyrazin-2-yl, 1,5-dimethylpyrazol-3-yl, 2-pyrimidinyl, 5-bromopyrimidin-2-yl, 5-methylpyrimidin-2-yl, 4,6-dimethylpyrimidin-2-yl, 3-pyridazinyl, or 6-methylpyridazin-3-yl, 4-fluoropyrid-2-yl, 4-chloropyrid-2-yl, 4-bromopyrid-2-yl, 4-methylpyrid-2-yl or 4-trifluoropyrid-2-yl.

Preference is also given to compounds of the formula (I) or salts thereof in which
(R⁶)ₙ is n substituents R⁶, where R⁶, in the case that n=1, or each of the substituents R⁶ independently of one another, in the case that n is greater than 1, is a radical halogen, cyano, (C₁-C₄)-alkyl, (C₁-C₄)-haloalkyl, phenyl-(C₁-C₄)alkyl, (C₃-C₆)-cycloalkyl-(C₁-C₄)alkyl, (C₁-C₄)-alkyl which is substituted by saturated or partially saturated heterocyclyl having 3 to 6 ring atoms and one to three ring heteroatoms selected from the group consisting of N, O and S, (C₁-C₄)-alkoxy, (C₁-C₄)-alkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkoxycarbonyl, (C₃-C₆)-cycloalkyl or saturated or partially saturated heterocyclyl having 3 to 6 ring atoms and one to three ring heteroatoms from the group consisting of N, O and S, and
n is 0, 1, 2 or 3, preferably 0 or 1.

Here, preference is also given to compounds of the formula (I) or salts thereof in which
(R⁶)ₙ is n substituents R⁶, where R⁶, in the case that n=1, or each of the substituents R⁶ independently of one another, in the case that n is greater than 1, is a radical halogen, such as fluorine, chlorine, bromine or iodine, methyl, ethyl, trifluoromethyl, benzyl, cyclopropylmethyl, methoxy, ethoxy, methylthio, ethylthio, methylsulfinyl, ethylsulfinyl, methylsulfonyl, ethylsulfonyl, methoxycarbonyl, ethoxycarbonyl, cyclopropyl or tetrahydrofuryl, and
n is 0, 1, 2 or 3, preferably 0 or 1.

Preference is given to compounds of the formula (I) or salts thereof in which a radical R⁶ which is attached to a ring nitrogen atom is a radical hydroxyl, amino, carbamoyl, (C₁-C₆)-alkyl, (C₁-C₆)-haloalkyl, (C₁-C₄)-alkoxy-(C₁-C₄)-alkyl, (C₁-C₄)-alkylthio-(C₁-C₄)-alkyl, mono- or di-[(C₁-C₄)-alkyl]aminoalkyl, hydroxy-(C₁-C₄)-alkyl, (C₁-C₄)-alkyl which is substituted
by optionally halogen-, cyano-, (C₁-C₄)-alkyl- or (C₁-C₄)-haloalkyl-substituted (C₃-C₆)-cycloalkyl,
by optionally halogen-, cyano-, (C₁-C₄)-alkyl-, (C₁-C₄)-haloalkyl- or (C₁-C₄)-alkoxy-substituted phenyl or
by optionally halogen-, cyano-, (C₁-C₄)-alkyl-, (C₁-C₄)-haloalkyl- or (C₁-C₄)-alkoxy-substituted heterocyclyl, preferably saturated or partially saturated heterocyclyl which contains in each case 3 to 6 ring atoms and one to three ring heteroatoms selected from the group consisting of N, O and S,
   or (C₂-C₆)-alkenyl, (C₂-C₆)-alkynyl, (C₁-C₆)-alkoxy, [(C₁-C₆)-alkoxy]carbonyl, [(C₁-C₆)-haloalkoxy]carbonyl, (C₁-C₆)-alkanoyl, (C₁-C₆)-haloalkanoyl, mono- or di-[(C₁-C₄)alkyl]aminocarbonyl, mono- or di-[(C₁-C₆)acyl]amino, mono- or di-[(C₁-C₄)alkyl]amino, N—[(C₁-C₆)-acyl]-N—[(C₁-C₆)alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₃-C₉)-cycloalkyl, (C₅-C₉)-cycloalkenyl,
where each of the two lastmentioned radicals is unsubstituted or substituted by one or more radicals selected from the group consisting of halogen, (C₁-C₄)-alkyl and (C₁-C₄)-haloalkyl,
   or optionally halogen-, cyano-, (C₁-C₄)-alkyl-, (C₁-C₄)-haloalkyl- or (C₁-C₄)-alkoxy-substituted heterocyclyl, preferably saturated or partially saturated heterocyclyl which contains in each case 3 to 6 ring atoms and one to three ring heteroatoms selected from the group consisting of N, O and S.

More preference is given to compounds of the formula (I) or salts thereof in which a radical R⁶ which is attached to a ring nitrogen atom is a radical (C₁-C₄)-alkyl, phenyl-(C₁-C₄)-alkyl, (C₃-C₆)-cycloalkyl-(C₁-C₄)alkyl, (C₁-C₄)-alkyl which is substituted by saturated heterocyclyl having 3 to 6 ring atoms and one to three ring heteroatoms from the group consisting of N, O and S, (C₁-C₄)-alkylsulfonyl, (C₁-C₄)-alkoxycarbonyl, (C₃-C₆)-cycloalkyl or saturated heterocyclyl having 3 to 6 ring atoms and one to three ring heteroatoms from the group consisting of N, O and S.

More preference is given to compounds of the formula (I) or salts thereof in which a radical R⁶ which is attached to a ring nitrogen atom is a radical methyl, ethyl, benzyl, cyclopropylmethyl, methylsulfonyl, ethylsulfonyl, methoxycarbonyl, ethoxycarbonyl, cyclopropyl or tetrahydrofuryl.

More preference is given to compounds of the formula (I) or salts thereof in which X² is a divalent group NH, NR⁶ or N, where the radical R⁶ specifically bound at nitrogen is as defined above, in particular as defined above as preferred.

More preference is given to compounds of the formula (I) or salts thereof in which X² is a divalent group NH, NR⁶ or N, where the radical R⁶ specifically bound at nitrogen is a radical methyl, ethyl, trifluoromethyl, benzyl, cyclopropylmethyl, methoxy, ethoxy, methylthio, ethylthio, methylsulfinyl, ethylsulfinyl, methylsulfonyl, ethylsulfonyl, methoxycarbonyl, ethoxycarbonyl, cyclopropyl or tetrahydrofuryl.

Preference is given to compounds of the formulae below or salts thereof in which the radicals R¹, R², R³, R⁴, R⁵, R⁶ and the divalent groups X¹, X², X³ and X⁴ and n are defined as in formula (I) or defined in accordance with the preferred meanings mentioned:

formula (Ia) [1-(1H-pyrazol-3-yl)pyrazole-4-acetic acid derivatives],
formula (Ib) [1-(1H-pyrazol-4-yl)pyrazole-4-acetic acid derivatives],
formula (Ic) [1-(1H-pyrazol-5-yl)pyrazole-4-acetic acid derivatives],
formula (Id) [1-(1H-imidazol-2-yl)pyrazole-4-acetic acid derivatives],
formula (Ie) [1-(1H-imidazol-5-yl)pyrazole-4-acetic acid derivatives],
formula (If) [1-(4H-1,2,4-triazol-3-yl)pyrazole-4-acetic acid derivatives],
formula (Ig) [1-(1H-1,2,3,4-tetrazol-5-yl)pyrazole-4-acetic acid derivatives],
formula (Ih) [1-(thien-3-yl)pyrazole-4-acetic acid derivatives],
formula (Ii) [1-(thien-2-yl)pyrazole-4-acetic acid derivatives],
formula (Ij) [1-(1,3-thiazol-2-yl)pyrazole-4-acetic acid derivatives],
formula (Ik) [1-(1,3-thiazol-4-yl)pyrazole-4-acetic acid derivatives],
formula (IL) [1-(1,2-thiazol-3-yl)pyrazole-4-acetic acid derivatives], (isothiazol-3-yl compounds)
formula (Im) [1-(1,3,4-thiadiazol-2-yl)pyrazole-4-acetic acid derivatives],
formula (In) [1-(1,2,4-thiadiazol-5-yl)pyrazole-4-acetic acid derivatives],

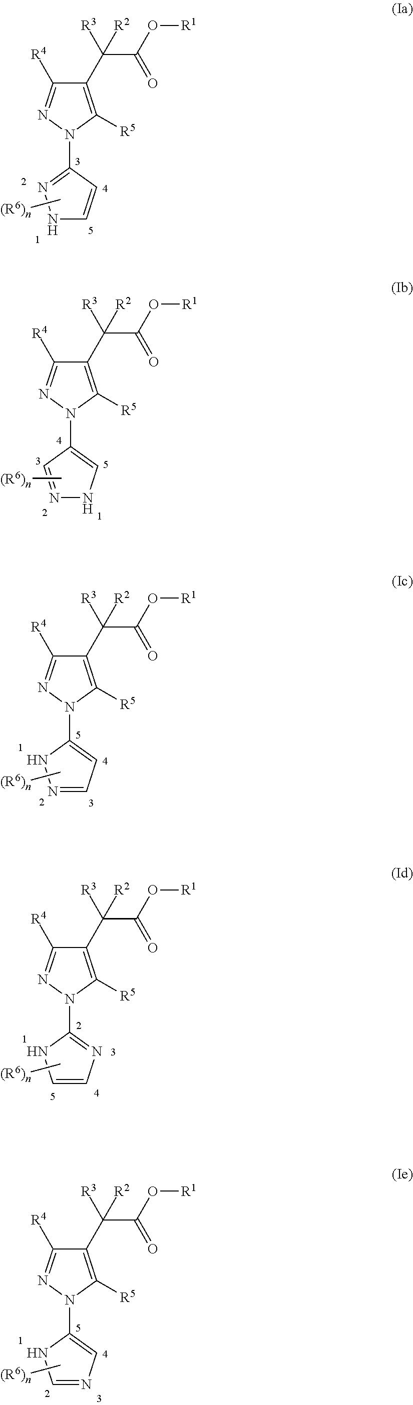

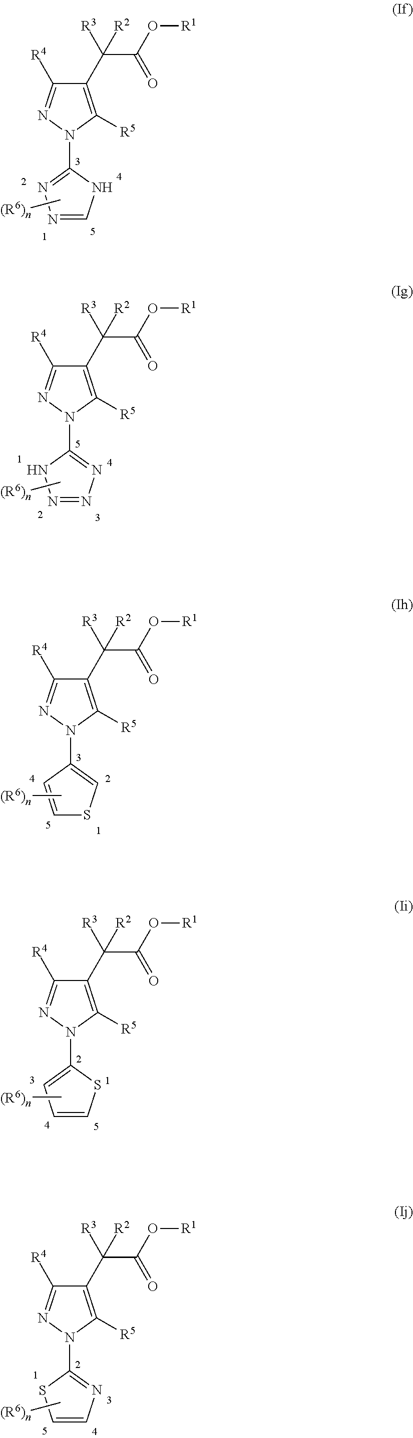

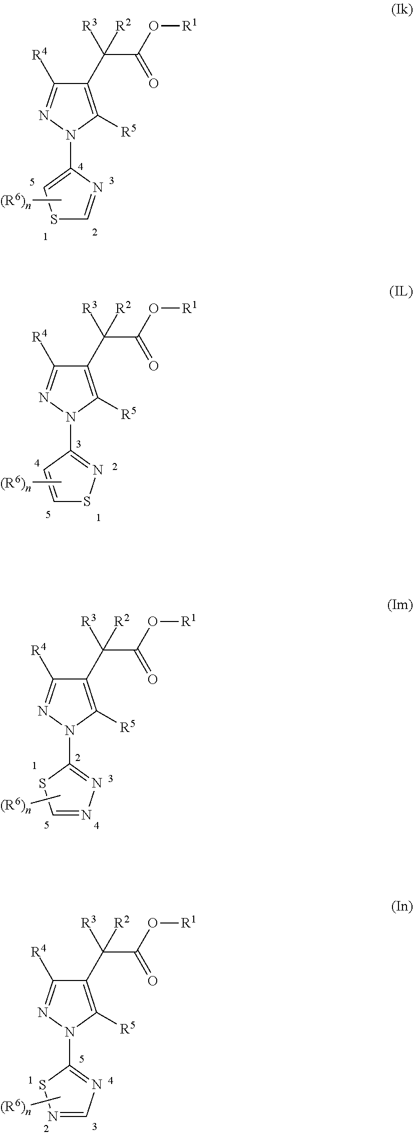

Preference is also given to compounds of the formula (I) or salts thereof in which the radicals R¹, R², R³, R⁴, R⁵, R⁶ and the divalent groups X¹, X², X³ and X⁴ and n have been selected according to two or more of the preferred meanings mentioned.

Here, particular preference is given to the compounds of the formula (I), (Ia), (Ib), (Ic), (Id), (Ie), (If), (Ig), (Ih), (ID, (Ik), (IL), (Im) or (In) or salts thereof in which one or more of the radicals R¹ to R⁶ have the radical meanings given in the example tables.

The compounds of the formula (I) according to the invention include all stereoisomers which can occur on the basis of the centers of asymmetry or double bonds in the molecule whose configuration is not designated specifically in the formula or which are not specified explicitly, and mixtures thereof, including the racemic compounds and the mixtures enriched partly with particular stereoisomers.

The invention also includes all tautomers, such as keto and enol tautomers, and their mixtures and salts, if appropriate functional groups are present.

The invention also provides processes for preparing the compounds of the formula (I) and/or their salts.

The compounds of the formula (I) according to the invention can be prepared by various alternative processes.

In the processes below, in some cases solvents are employed. In this context, “inert solvents” refers in each case to solvents which are inert under the particular reaction conditions, but which do not have to be inert under any reaction conditions.

In the processes a), b), c), d), e), f), g), h), i) below, the reactions described can alternatively also be carried out in a microwave oven.
(a) To prepare compounds of the formula (I) or salts thereof in which R¹, R², R³, R⁴, R⁵, (R⁶)ₙ, X¹, X², X³, X⁴ and n have the meanings indicated above for formula (I), according to preparation process (a) [=variant (a)] a compound of the formula (II)

H₂N—NH-Q (II)
in which Q is a radical of the formula (Q)

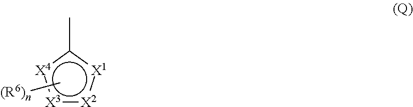

in which X¹, X², X³, X⁴, (R⁶)ₙ and n are as defined for formula (I)
is reacted with a compound of the formula (III)

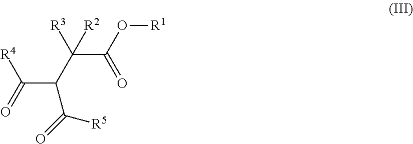

in which R¹, R², R³, R⁴ and R⁵ are as defined for formula (I)
to give the compound of the formula (I) or a salt thereof.

The substituted 1,3-dicarbonyl compounds of the formula (III) used as starting materials in the process (a) according to the invention for preparing compounds of the formula (I) are preferably those where the radicals R¹, R², R³, R⁴ and R⁵ have the preferred meanings already indicated above in connection with the description of the compounds of the formula (I) according to the invention as being preferred. Accordingly, the substituted heteroarylhydrazines of the formula (II) used as starting materials in the process (a) according to the invention for preparing compounds of the formula (I) preferably also have those meanings for (R⁶)ₙ which have already been indicated above in connection with the description of the compounds of the formula (I) according to the invention as being preferred for (R⁶)ₙ, and in particular preferred as a function of the radicals Het.

Hydrazines of the formula (II) or salts thereof as starting materials are known and/or can be prepared by known processes (cf., for example, Methoden der organischen Chemie (Houben-Weyl, D. Klamann, Ed.) volume E16a, part 1, p. 421 ff., Georg Thieme Verlag, Stuttgart 1990 and the literature cited therein; J. Am. Chem. Soc., 1954, 76, 596; Monatshefte für Chemie 1988, 119, 333; J. Heterocyclic Chem. 1988, 25, 1055; J. Heterocyclic Chem. 1989, 26, 475; Heterocycles 1994, 37, 379).

The reaction of the compounds of the formulae (II) and (III) can be carried out without catalyst or in the presence of catalysts, for example of an acid as catalyst, preferably in an organic solvent, such as tetrahydrofuran (THF), dioxane, acetonitrile, dimethylformamide (DMF), methanol and ethanol, at temperatures between 20° C. and the boiling point of the solvent, preferably at from 50° C. to 150° C. If acid addition salts of the formula (II) are used, these are generally liberated in situ with the aid of a base. Suitable bases or basic catalysts are alkali metal hydroxides, alkali metal hydrides, alkali metal carbonates, alkali metal bicarbonates, alkali metal alkoxides, alkaline earth metal hydroxides, alkaline earth metal hydrides, alkaline earth metal carbonates or organic bases, such as triethylamine, diisopropylethylamine or 1,8-diazabicyclo[5.4.0]undec-7-ene (DBU).

Analogous processes have been described in the literature, for example in WO 2004/037793.
(b) If R¹ in formula (I) is different from hydrogen, the preparation process [=variant (b)] is characterized in that a compound of the formula (I′)

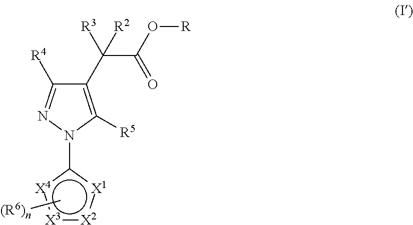

in which R², R³, R⁴, R⁵, R⁶, X¹, X², X³, X⁴ and n are as defined for formula (I) and
R is a radical which is different from the radical R¹ and different from hydrogen and is selected from the group of radicals as defined for R¹,
   or an anhydride, acid halide or an activated ester of the compound of the formula (I′) in which R═H,
is reacted with a compound of the formula (IV)

R¹—OH (IV)
in which R¹ is as defined for formula (I)
to give the compound of the formula (I) or
(c) if R¹ in formula (I) is different from hydrogen, the preparation process [=variant (c)] is characterized in that a compound of the formula (I″)

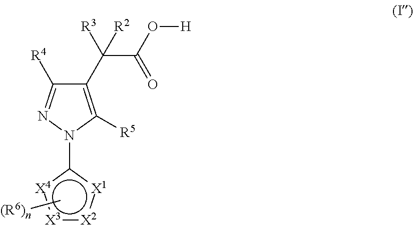

in which R², R³, R⁴, R⁵, R⁶, X¹, X², X³, X⁴ and n are as defined for formula (I) is, if appropriate after activation of the acid group, reacted (esterified) with a compound of the formula (IV)

R¹—OH (IV)
in which R¹ is as defined for formula (I)
to give the compound of the formula (I) or
(d) if the compound of the formula (I) in which R¹═H or its salt is prepared, the preparation process [=variant (d)] is characterized in that a compound of the formula (I′) [see definition in variant (b)] is hydrolyzed to give the compound of the formula (I) or its salt.

The starting materials of the formulae (II), (III) and (IV) are generally known or can be prepared analogously to known processes.

The reaction of the compounds of the formulae (I′) and (IV) can be carried out using standard methods of transesterification or esterification via activated carboxylic acids.

The reaction of the compounds of the formulae (I″) and (IV) can be carried out using standard methods of esterification or, if appropriate, via activated carboxylic acids. The preparation of compounds of the formula (I″) from compounds (I′) can be carried out using standard methods of hydrolysis.

Starting Materials for Variants (a) and (b)

(a1) The compounds of the formula (III) can be prepared, for example, by reacting a dicarbonyl compound of the formula (V)

R⁴—CO—CH₂—CO—R⁵ (V)

with a compound of the formula (VI)

R²R³C(Hal)-CO—OR¹ (VI)
where R¹, R², R³, R⁴ and R⁵ are defined as for formula (III) and R¹ is preferably methyl or ethyl, and Hal is a leaving group, preferably a reactive halogen, such as a chlorine atom or, in particular, a bromine atom, or else p-toluenesulfonyl (tosyl) or methylsulfonyl (mesyl).

The compounds of the formula (I) according to the invention can be prepared by known methods analogously to the processes a) to d) and (al) mentioned, as described, for example, in Methoden der organischen Chemie [Methods of Organic Chemistry] (Houben-Weyl, E. Schaumann, Ed.) volume Ebb, Hetarenes III, part 2, pp. 399-710, Georg Thieme Verlag, Stuttgart 1994 and the literature cited therein, where the syntheses according to Methoden der organischen Chemie [Methods of Organic Chemistry] (Houben-Weyl, E. Schaumann, Ed.) volume Ebb, Hetarenes III, part 2, p. 420 ff., Georg Thieme Verlag, Stuttgart 1994 and the literature cited therein; Synthesis, 1986, 409; J. Chinese Chem. Soc., 2001, 48, 45 and in particular U.S. Pat. No. 4,146,721, DE2141124, DOS1946370 and Justus Liebigs Ann. Chem. 1973, 1919 are of particular interest.

(a2) The compounds of the formula (V) can also be prepared, for example, by reacting a compound of the formula (VII)

R⁵—CO—OR⁷ (VII)

with a compound of the formula (VIII)

CH₃—CO—R⁴ (VIII)

where R⁴ and R⁵ are as defined for formula (I), R⁷ is (C₁-C₆)-alkyl, preferably methyl or ethyl, in the presence of a suitable organic base, such as, for example, sodium methoxide or sodium ethoxide, in a suitable solvent, for example methanol, ethanol or, preferably, tetrahydrofuran, in a temperature range between −10 and 50° C., preferably 0° C., and, if appropriate, under an atmosphere of inert gas, for example nitrogen.

Reactions analogous to the reaction have been described in the literature, for example Supramolecular Chemistry (2003), 15(7-8), 529-547; J. Am. Chem. Soc. (1951), 73 5614-16; J. of Med. Chem. (1990), 33(7), 1859-65; WO 00/08002.

(a3) Alternatively, compounds of the formula (V) can also be obtained by reacting a compound of the formula (IX)

R⁴—CO—OR⁷ (IX)

with a compound of the formula (X)

CH₃—CO—R⁵ (X)

under conditions analogous to those described above under (a2),
where R⁴ and R⁵ are as defined for formula (I), R⁷ is (C₁-C₆)-alkyl, preferably methyl or ethyl.

Reactions analogous to the reaction have been described in the literature, for example in J. Am. Chem. Soc. (1950), 72 1352-6.
(e) To prepare a compound of the formula (I) or a salt thereof, it is also possible, for example, to react a compound of the formula (XI) with a boron derivative of the formula (XII) in the presence of a suitable Cu(I) or Cu(II) salt and an organic base, if appropriate in a solvent, as shown in the scheme below:

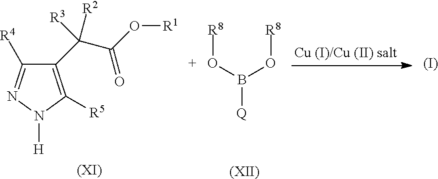

where in formula (XI) R¹, R², R³, R⁴, R⁵ have the meaning given above for formula (I) and in formula (XII) R⁸ is H or (C₁-C₆)-alkyl, preferably methyl, or both alkyl radicals R⁸ are attached cyclically to one another and Q is a radical of the formula (Q)

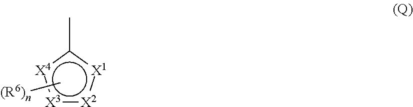

in which X¹, X², X³, X⁴, (R⁶)ₙ and n are as defined for formula (I).

The reaction is carried out in the presence of a suitable inorganic or organic copper(I) or copper(II) salt, preferably CuI, Cu₂O, particularly preferably copper(II) acetate [Cu(OAc)₂], employed as 0.1 to 1.5 equivalents, using more than one equivalent of boron derivative (XII), preferably between 1.5 and 2.5 equivalents. If the copper catalyst is used in non-stoichiometric amounts, the addition of a mild oxidizing agent, for example 2,2,6,6-tetramethylpiperidine-1-oxyl (TEMPO), pyridine oxide, oxygen or dry air, may be helpful.

To this end, a suitable organic base such as, for example, pyridine, triethylamine or potassium tert-butoxide is added and, to make the transmetallation more efficient, a source of fluoride anions, preferably cesium fluoride.

The reaction is carried out in a suitable solvent, preferably a halogenated solvent, for example trichloromethane or, preferably, dichloromethane, in a temperature range between 0 and 40° C., preferably between 20 and 30° C., and, if appropriate, under an atmosphere of inert gas, for example nitrogen, until the reaction has gone to completion, which may in some cases require long reaction times.

Analogous methods for copper-induced C—N couplings have been described in the literature, for example in Tet. Lett. 1998, 39, 2941; Tet. Lett. 1998, 39, 2933; Tet. Lett. 44 (2003) 3863-3865; J. Comb. Chem. 2004, 6, 385-390; Tet. Lett. 41 (2000) 9053 to 9057.

Analogous methods for copper-induced C—N couplings in the presence of fluoride anions have been described in the literature, for example in Eur. J. Org. Chem. 2007, 1318-1323 and Org. Lett. 2007, 9 (5), 761.

Analogous methods for catalytic copper-induced C—N couplings have been described in the literature, for example in Tet. Lett. 2001, 3415; Org. Lett. 2003, 5 (23), 4397 and Org. Lett. 2001, 3 (13), 2077.

Analogous methods for copper-induced C—N couplings in a microwave reactor have been described in the literature, for example J. Comb. Chem. 358 2008, 10, 358-360.

Compounds of the formula (XI) can be prepared by processes known to the person skilled in the art, for example by reacting a compound of the formula (III), in which R¹, R², R³, R⁴ and R⁵ are as defined for formula (I), with hydrazine (hydrate) as described in Can. J. Chem. 2001, 79 (2), 183-194.

Compounds of the formula (XII) are known to the person skilled in the art, and some are commercially available or can be prepared by processes known to the person skilled in the art, for example as described in a) Science of Synthesis, Houben-Weyl (Methods of Molecular Transformations), Category 2, Volume 6, Ed. E. Schaumann; b) Houben-Weyl (Methoden der organische Chemie [Methods of Organic Chemistry]), Volume 13, Organoborverbindungen [Organoboron Compounds] I-Part 3a, Ed. E. Schaumann.
(f) To prepare a compound of the formula (I) or a salt thereof, it is also possible, for example, to react a compound of the formula (XI) with a compound of the formula (XIII) in the presence of a suitable catalyst/ligand system with a suitable base and in a suitable solvent, as shown in the scheme below:

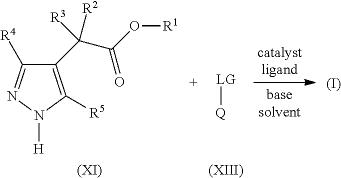

where in formula (XI) R¹, R², R³, R⁴, R⁵ have the meaning given above for formula (I) and in formula (XIII) LG is a leaving group, for example, chlorine, bromine, iodine, phenylsulfonate, tosylate or triflate, and Q is a radical of the formula (Q)

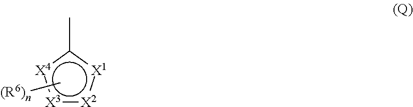

in which X¹, X², X³, X⁴, (R⁶)ₙ and n are as defined for formula (I).

Compounds of the formula (XIII) can be reacted with compounds of the formula (XI) in the presence of a suitable catalyst/ligand system to give compounds of the formula (I). The reaction is preferably carried out using a suitable inorganic or organic copper(I) or copper(II) salt, preferably, for example, Cu(OAc)₂ (copper diacetate), Cu(Acac)₂ (copper diacetylacetate), CuI (copper iodide), CuBr (copper bromide), Cu₂O (copper(I) oxide), [Cu(OH)TMEDA]₂Cl₂ (a complex copper salt, TMEDA is “tetramethylethylenediamine”), or CuO (copper(II) oxide), using a suitable ligand such as, for example, L-proline, N,N′-dimethylethane-1,2-diamine, trans-N,N′-dimethylcyclohexane-1,2-diamine, dimethylglycine, salicylaldoxime, 1,1′-binaphthalene-2,2′-diol (BINOL) and an organic or inorganic base such as, for example, triethylamine, pyridine, 2,6-lutidine, cesium carbonate, potassium carbonate, potassium phosphate, if appropriate in a solvent, such as, for example, toluene, 1,4-dioxane, dichloromethane, dimethylformamide, dimethylacetamide, acetonitrile.

The reaction can also be carried out in the presence of a suitable palladium catalyst, for example palladium(II) acetate, or dipalladium-tri-[(1E,4E)-1,5-diphenylpenta-1,4-dien-3-one], using a phosphine ligand such as, for example, 2,2′-bis(diphenylphospino)-1,1′-binaphthyl (BINAP), 1,1′-diphenylphosphinoferrocene (DPPF), 2-di-tert-butylphosphinobiphenyl (JohnPhos), 2-dicyclohexylphosphino-2′-dimethylaminobiphenyl (DavePhos), 2-dicyclohexylphosphino-2′,4′,6′-triisopropylbiphenyl (XPhos), 2-dicyclohexylphosphino-2′-methylbiphenyl (MePhos), 4,5-bis(diphenylphosphino)xanthene (XANTPHOS), tri-tert-butylphosphonium tetrafluoroborate or di-tert-butylphosphinic acid and an inorganic base such as, for example, cesium carbonate, potassium carbonate, potassium phosphate, if appropriate in a solvent, such as, for example, toluene, 1,4-dioxane, dichloromethane, dimethylformamide, dimethylacetamide or acetonitrile or mixtures thereof.

The reaction can also be carried out in the presence of a suitable Fe(III) complex, such as, for example, iron(III) oxide, iron trichloride or iron triacetylacetonate, using a suitable ligand such as, for example, L-proline, N,N′-dimethylethane-1,2-diamine, trans-N,N¹-dimethylcyclohexane-1,2-diamine or dimethylglycine, and an inorganic base such as, for example, cesium carbonate, potassium carbonate, potassium phosphate, if appropriate in a solvent, such as, for example, toluene, 1,4-dioxane, dichloromethane, dimethylformamide, dimethylacetamide or acetonitrile or mixtures thereof.

The reaction can also be carried out in the presence of a mixture of the palladium- and copper- or iron- and copper-based catalysts.

The reaction is generally carried out in the presence of more than one equivalent of a compound of the formula (XI), preferably from 1 to 2 equivalents, or in the presence of more than one equivalent of a compound of the formula (XIII), preferably from 1 to 1.5 equivalents.

The reaction is carried out, for example, in a temperature range between 0 and 150° C., preferably between 60 and 120° C., and, if appropriate, under an atmosphere of inert gas, for example nitrogen, until the conversion is complete, where in some cases long reaction times may be required.

Analogous methods for copper-induced C—N couplings are described in the literature, for example Tet. Lett. 49 (2008) 948-951; Eur. J. Org. Chem. 2004, 695, 709; J. Org. Chem. 2007, 72, 2737-2743; Heterocycles, 61, 2003, 505-512; Heterocycles, 48 (11), 1998, 2225; J. Am. Chem. Soc. 2002, 124, 11684-11688; J. Org. Chem. 2004, 69, 5578-5587; J. Org. Chem. 2007, 72, 8535-8538; Org. Lett. 2000, 2 (9), 1233-1236; Journal of Molecular Catalysis A: Chemical (2006), 256(1-2), 256-260; Acc. Chem. Res. (2008), 41(11), 1450-1460, J. Mol. Catal. A: Chemical 256 (2006) 256-260).

Analogous methods for palladium-induced C—N couplings are described in the literature, for example in J. Org. Chem. 2001, 66, 8677; J. Org. Chem. 1999, 64, 6019-6022; Angew. Chem. Int. Ed. 2005, 44, 1371-1375; Heterocycles, 48, 11, 1998, 847; Tetrahedron 61 (2005) 2931-2939; Angew. Chem. Int. Ed. 2006, 45, 6523-6527.

Analogous methods for iron-induced C—N couplings are described in the literature, for example in Angew. Chem. Int. Ed. 2007, 46, 934; Angew. Chem. Int. Ed. 2007, 46, 8862-8865.

Analogous methods for copper/iron-induced C—N couplings are described in the literature, for example in Angew. Chem. Int. Ed. 2007, 46, 934; Angew. Chem. Int. Ed. 39, 1998, 5617-5620.

Analogous methods for copper-induced C—N couplings in a microwave reactor have been described in the literature, for example J. Comb. Chem. 358 2008, 10, 358-360.

Compounds of the formula (XI) can be prepared by processes known to the person skilled in the art, for example by reacting a compound of the formula (III), in which R¹, R², R³, R⁴ and R⁵ are as defined for formula (I), with hydrazine (hydrate) as described in Can. J. Chem. 2001, 79 (2), 183-194.

Compounds of the formula (XIII) are known to the person skilled in the art, and some of them are commercially available or can be prepared by processes known to the person skilled in the art, for example as described in Science of Synthesis, Houben-Weyl (Methods of Molecular Transformations), Category 2, Volume 16, Ed. E. Schaumann.
(g) Another possible starting material for preparing a compound of the formula (I) mentioned or a salt thereof is, for example, a compound of the formula (XV), which is prepared by reacting a compound of the formula (XIII) in which R⁶ has the meaning given above for formula (I) with benzophenone hydrazone (XIV) in the presence of a suitable catalyst/ligand system with a suitable base and in a suitable solvent. Compounds of the formula (XV) give, using a compound of the formula (III) in the presence of an acid, if appropriate in a solvent, the compound of the formula (I), as shown in the scheme below:

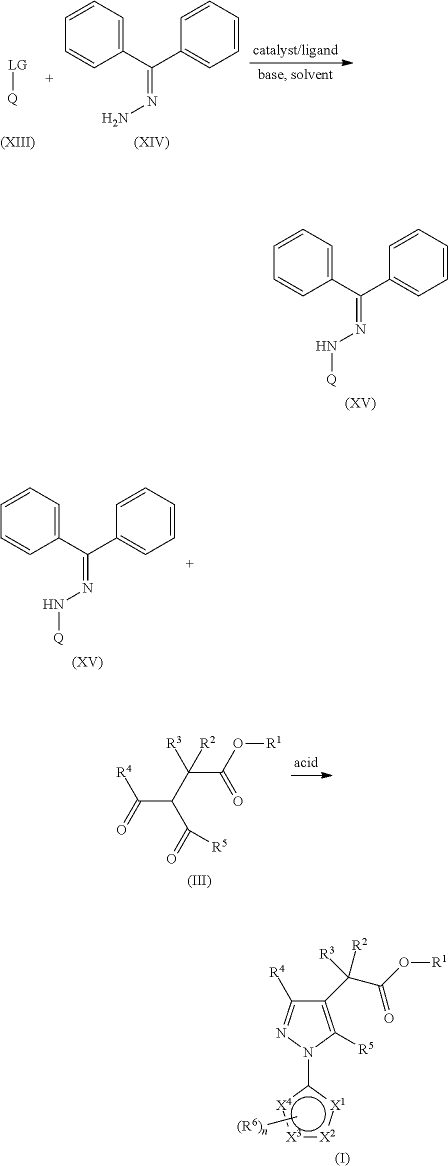

Here, R¹, R², R³, R⁴, R⁵ have the meaning given above for formula (I), LG is a leaving group, suitable leaving groups being, for example, chlorine, bromine, iodine, phenylsulfonate, tosylate or triflate, and Q is a radical of the formula (Q)

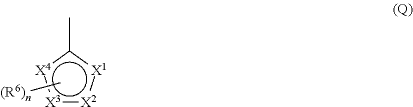

in which X¹, X², X³, X⁴, (R⁶)ₙ and n are as defined for formula (I).

Compounds of the formula (XIII) can be reacted with benzophenone hydrazone (XIV) in the presence of a catalyst and a suitable catalyst/ligand system to give compounds of the formula (XV). The reaction is preferably carried out using a palladium catalyst, for example palladium(II) acetate, with a phosphine ligand such as, for example, 2,2′-bis(diphenylphospino)-1,1′-binaphthyl (BINAP), 1,1′-diphenylphosphinoferrocene (DPPF), 2-di-tert-butylphosphinobiphenyl (JohnPhos), 2-dicyclohexylphosphino-2′-dimethylaminobiphenyl (DavePhos), 2-dicyclohexylphosphino-2′,4′,6′-triisopropylbiphenyl (XPhos), 2-dicyclohexylphosphino-2′-methylbiphenyl (MePhos), 4,5-bis(diphenylphosphino)xanthene (XANTPHOS). The use of a base, for example sodium tert-butoxide, is advantageous. The reaction is generally carried out under an atmosphere of inert gas, for example nitrogen, with exclusion of water in a suitable solvent, for example toluene.

Benzophenone hydrazone is commercially available.

Compounds of the formula (XV) can directly be reacted further with compounds of the formula (III) to give compounds of the formula (I). The reaction is carried out in the presence of a suitable inorganic or organic (non)aqueous acid, preferably p-toluenesulfonic acid, sulfuric acid, particularly preferably hydrochloric acid, where for example from 1 to 10, preferably from 3 to 7, particularly preferably about 5 equivalents of the acid are employed.

The reaction is carried out, for example, in a suitable solvent, for example diethyl ether, dioxane or preferably tetrahydrofuran, in a temperature range between 0 and 80° C., preferably 50° C., and, if appropriate under an atmosphere of inert gas, for example nitrogen.

Analogous cyclization reactions of a hydrazone with a 1,3-diketone to give a pyrazole are described in the literature, for example in WO-A-2001/32627; Angew. Chem. 110 (1998) 2249-2252; Tet. Lett. 43 (2002) 2171-2173; J. Am. Chem. Soc. 1981, 103, 7743-7752; Organic Process Research and Development 2004, 8, 1065-1071; Tet. Lett. 45 (2004) 5935-5937; WO-A-2007/064872, U.S. Pat. No. 6,489,512, WO-A-2006/114213.

Compounds of the formula (XV) can also be converted into compounds of the formula (II), for example by processes known to the person skilled in the art, in the presence of acid, preferably under partially aqueous conditions. The compounds of the formula (II) can react further according to process (a) mentioned above to give compounds of the formula (I).

Compounds of the formula (XIII) are known to the person skilled in the art, and some of them are commercially available or can be prepared according to processes known to the person skilled in the art, for example as described in Science of Synthesis, Houben-Weyl (Methods of Molecular Transformations), Category 2, Volume 16, Ed. E. Schaumann.

Compounds of the formula (XV) can be prepared as described, for example, in Tet. Lett. 45 (2004) 5935-5937; Angew. Chem. Int. Ed. 2006, 45, 6523-6527; J. Am. Chem. Soc. (2003) 125, 13978-13980; J. Am. Chem. Soc. (2003), 125, 6653-6655; Org. Lett. 3 (9) (2001) 1351; Tet. Lett. 45 (2004) 5935-5937; Tetr. Lett. 43 (2002) 2171-2173; Angew. Chem. Int. Ed. 1998, 37 (15) 2090; WO2001/32627; J. Am. Chem. Soc. (1998) 120, 6621; WO-2007/064872; U.S. Pat. No. 6,489,512; WO-2006/114213; US-2005/0192294, J. Am. Chem. Soc. 2003, 125, 6653-6655; Org. Lett. 2001, 3 (9), 1351-1354.
(h) To prepare a compound of the formula (I) mentioned or salts thereof, it is also possible to react, for example, a compound of the formula (XVI) where R⁶ is as defined for formula (I) with di-tert-butyl azodicarboxylate (DBAD, XVII) in the presence of a suitable copper salt in a suitable solvent to give a compound of the formula (XVIII) in which R⁶ is as defined for formula (I). This gives compounds of the formula (II) and salts thereof in which R⁶ is as defined for formula (I) which can be reacted according to process (a) to give compounds of the formula (I) in which R¹, R², R³, R⁴, R⁵ and R⁶ are as defined for formula (I), as shown in the scheme below:

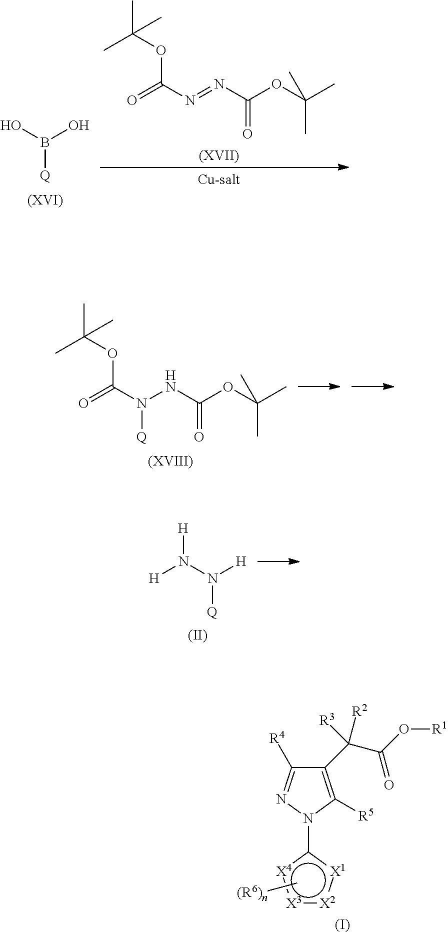

Here, Q is a radical of the formula (Q)

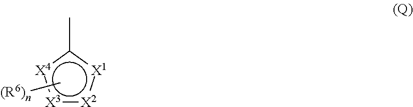

in which X¹, X², X³, X⁴, (R⁶)ₙ and n are as defined for formula (I).

The reaction is carried out, for example, in the presence of a suitable inorganic copper salt, for example Cu(OAc)₂ (copper diacetate) or its monohydrate Cu(OAc)₂.H₂O, in a suitable solvent, for example in an alcohol, such as methanol, in a temperature range between 0 and 40° C., preferably 20-25° C., and, if appropriate under an atmosphere of inert gas, for example nitrogen.

Analogous reactions using commercially available di-tert-butyl(E)-diazene-1,2-dicarboxylate (DTBAD) are described in the literature, for example Org. Lett. (2006) 8 (1), 43-45; J. Org. Chem. 2005, 70, 8631-8634.

Compounds of the formula (XVI) are commercially available and/or can be prepared by processes known to the person skilled in the art, for example as described in a) Science of Synthesis, Houben-Weyl (Methods of Molecular Transformations), Category 2, Volume 6, Ed. E. Schaumann; b) Houben-Weyl (Methoden der organischen Chemie), Volume 13, Organoboron compounds I-Part 3a, Ed. E. Schaumann.

Compounds of the formula (XVIII) can be converted into compounds of the formula (II) by processes known to the person skilled in the art as described, for example, in J. Med. Chem. 1998, 41, 2858-2871; Tetrahedron 44 (17), 5525 (1988); J. Med. Chem. 1996, 39, 1172-1188; J. Org. Chem. 2004, 69, 5778-5781. Compounds of the formula (II) or salts thereof can be converted by process (a) mentioned above into compounds of the formula (I).
(i) To prepare a compound of the formula (I) mentioned or salts thereof, it is also possible to react, for example, a compound of the formula (XIX) in which R⁶ is as defined for formula (I) and LG′ is a suitable (leaving) group, suitable groups being, for example, bromine and iodine, with a suitable metal or a suitable transmetallation reagent to give a compound of the formula (XX) which for its part is reacted with di-tert-butyl (E)-diazene-1,2-dicarboxylate (DTBAD, XVII) in the presence of a suitable solvent to give a compound of the formula (XVIII) in which R⁶ is as defined for formula (I). This gives compounds of the formula (II) or salts thereof in which R⁶ is as defined for formula (I) which can be reacted according to process (a) to give compounds of the formula (I) in which R¹, R², R³, R⁴, R⁵ and R⁶ are as defined for formula (I), as shown in the scheme below:

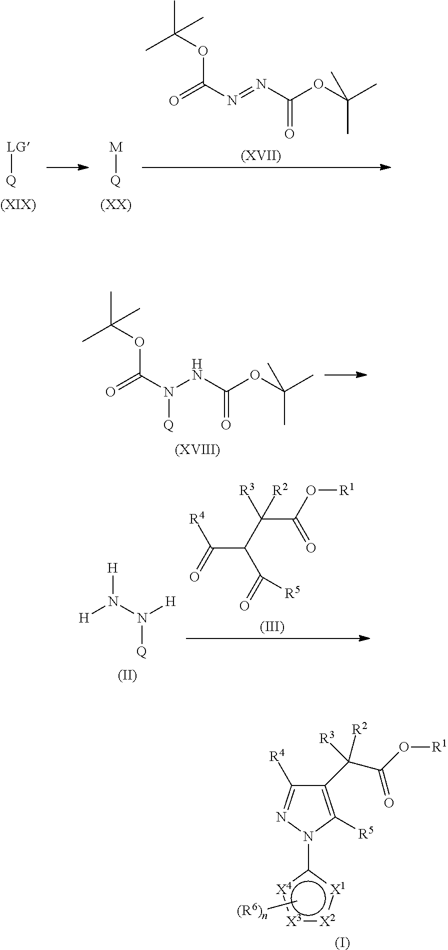

Here, Q has the meaning already mentioned for the above scheme.

The conversion into a compound of the formula (XX) is carried out, for example, in the presence of a suitable transmetallation reagent, for example an alkyllithium base, preferably BuLi (butyllithium); or a metal, preferably Li, Mg or Zn. The resulting nucleophile is reacted further with di-tert-butyl(E)-diazene-1,2-dicarboxylate (DTBAD, XVIII) to give a compound of the formula (XVIII). Analogous reactions using commercially available di-tert-butyl azodicarboxylate (DBAD) are described in the literature, for example Tet. Lett. 1987, 28 (42), 4933; Tet. Lett. 39 (1998) 9157-9160.

Compounds of the formula (XIX) are commercially available and/or can be prepared by processes known to the person skilled in the art, for example as described in Science of Synthesis, Houben-Weyl (Methods of Molecular Transformations), Category 2, Volume 16, Ed. E. Schaumann.

Compounds of the formula (XVIII) can be converted into compounds of the formula (II) by processes known to the person skilled in the art as described, for example, in J. Med. Chem. 1998, 41, 2858-2871; Tetrahedron 44 (17), 5525 (1988); J. Med. Chem. 1996, 39, 1172-1188; J. Org. Chem. 2004, 69, 5778-5781. Compounds of the formula (II) or salts thereof can be converted by process a) mentioned above into compounds of the formula (I).

The starting materials of the formula (III) can be obtained by generally known processes by alkylation of appropriate 1,3-diketones with 2-halogenated acetic acid derivatives, for example bromoacetic acid derivatives (cf., for example, DE-OS 1946370, p. 13). The 1,3-diketones (V) used as starting materials for this purpose can be prepared by the abovementioned process f) or are commercially available or known and/or can be prepared by known methods (see, for example, U.S. Pat. No. 4,146,721, DE2141124, DOS1946370 or J. Am. Chem. Soc., 1948, 70, 4023; Justus Liebigs Ann. Chem. 1973, 1919; Justus Liebigs Ann. Chem. 1976, 13; J. Chem. Soc. Perkin Trans. 2, 1993, 6, 1067; Heteroatom Chemistry, 1997, 8, 147).

Also possible for preparing enantiomers of the compounds (I) are customary methods for racemate separation (cf. textbooks of stereochemistry), for example following processes for separating mixtures into diastereomers, for example physical processes, such as crystallization, chromatographic processes, in particular column chromatography and high-pressure liquid chromatography, distillation, if appropriate under reduced pressure, extraction and other processes, it is possible to separate remaining mixtures of enantiomers, generally by chromatographic separation on chiral solid phases. Suitable for preparative amounts or on an industrial scale are processes such as the crystallization of diastereomeric salts which can be obtained from the compounds (I) using optically active acids and, if appropriate, provided that acidic groups are present, using optically active bases.

Optically active acids which are suitable for racemate separation by crystallization of diastereomeric salts are, for example, camphorsulfonic acid, camphoric acid, bromocamphorsulfonic acid, quinic acid, tartaric acid, dibenzoyltartaric acid and other analogous acids; suitable optically active bases are, for example, quinine, cinchonine, quinidine, brucine, 1-phenylethylamine and other analogous bases.

The crystallizations are then in most cases carried out in aqueous or aqueous-organic solvents, where the diastereomer which is less soluble precipitates first, if appropriate after seeding. One enantiomer of the compound of the formula (I) is then liberated from the precipitated salt, or the other is liberated from the crystals, by acidification or using a base.

The following acids are suitable for preparing the acid addition salts of the compounds of the formula (I): hydrohalic acids, such as hydrochloric acid or hydrobromic acid, furthermore phosphoric acid, nitric acid, sulfuric acid, mono- or bifunctional carboxylic acids and hydroxycarboxylic acids, such as acetic acid, maleic acid, succinic acid, fumaric acid, tartaric acid, citric acid, salicylic acid, sorbic acid, or lactic acid, and also sulfonic acids, such as p-toluenesulfonic acid or 1,5-naphthalenedisulfonic acid. The acid addition compounds of the formula (I) can be obtained in a simple manner by the customary methods for forming salts, for example by dissolving a compound of the formula (I) in a suitable organic solvent, such as, for example, methanol, acetone, methylene chloride or benzene, and adding the acid at temperatures of from 0 to 100° C., and they can be isolated in a known manner, for example by filtration, and, if appropriate, purified by washing with an inert organic solvent.

The base addition salts of the compounds of the formula (I) are preferably prepared in inert polar solvents, such as, for example, water, methanol or acetone, at temperatures of from 0 to 100° C. Examples of bases which are suitable for the preparation of the salts according to the invention are alkali metal carbonates, such as potassium carbonate, alkali metal hydroxides and alkaline earth metal hydroxides, for example NaOH or KOH, alkali metal hydrides and alkaline earth metal hydrides, for example NaH, alkali metal alkoxides and alkaline earth metal alkoxides, for example sodium methoxide or potassium tert-butoxide, or ammonia, ethanolamine or quaternary ammonium hydroxide of the formula [NRR′R″R′″]⁺OH⁻.

What is meant by the “inert solvents” referred to in the above process variants are in each case solvents which are inert under the particular reaction conditions but need not be inert under all reaction conditions.

Collections of compounds of the formula (I) and/or salts thereof which can be synthesized by the aforementioned reactions can also be prepared in a parallel manner, it being possible for this to take place in a manual, partly automated or completely automated manner. In this connection, it is, for example, possible to automate the reaction procedure, the work-up or the purification of the products and/or intermediates. Overall, this is understood as meaning a procedure as described, for example, by D. Tiebes in Combinatorial Chemistry—Synthesis, Analysis, Screening (editor Gunther Jung), Verlag Wiley 1999, on pages 1 to 34.

For the parallel reaction procedure and work-up, it is possible to use a series of commercially available instruments, for example Calypso reaction blocks from Barnstead International, Dubuque, Iowa 52004-0797, USA or reaction stations from Radleys, Shirehill, Saffron Walden, Essex, CB 11 3AZ, England or MuItiPROBE Automated Workstations from Perkin Elmer, Waltham, Mass. 02451, USA. For the parallel purification of compounds of the formula (I) and salts thereof or of intermediates produced during the preparation, there are available, inter alia, chromatography apparatuses, for example from ISCO, Inc., 4700 Superior Street, Lincoln, Nebr. 68504, USA.

The apparatuses listed lead to a modular procedure in which the individual process steps are automated, but between the process steps manual operations have to be carried out. This can be circumvented by using partly or completely integrated automation systems in which the respective automation modules are operated, for example, by robots. Automation systems of this type can be acquired, for example, from Caliper, Hopkinton, Mass. 01748, USA.

The implementation of single or several synthesis steps can be supported through the use of polymer-supported reagents/scavenger resins. The specialist literature describes a series of experimental protocols, for example in ChemFiles, Vol. 4, No. 1, Polymer-Supported Scavengers and Reagents for Solution-Phase Synthesis (Sigma-Aldrich).

Besides the methods described here, the preparation of compounds of the formula (I) and salts thereof can take place completely or partially by solid-phase supported methods. For this purpose, individual intermediates or all intermediates in the synthesis or a synthesis adapted for the corresponding procedure are bonded to a synthesis resin. Solid-phase supported synthesis methods are sufficiently described in the specialist literature, e.g. Barry A. Bunin in “The Combinatorial Index”, Verlag Academic Press, 1998 and Combinatorial Chemistry—Synthesis, Analysis, Screening (editor Gunther Jung), Verlag Wiley, 1999. The use of solid-phase supported synthesis methods permits a series of protocols known in the literature, which again can be carried out manually or in an automated manner. The reactions can be carried out, for example, by means of IROR1 technology in microreactors from Nexus Biosystems, 12140 Community Road, Poway, Calif. 92064, USA.

Both on a solid phase and in liquid phase can the procedure of individual or several synthesis steps be supported through the use of microwave technology. The specialist literature describes a series of experimental protocols, for example in Microwaves in Organic and Medicinal Chemistry (editor C. O. Kappe and A. Stadler), Verlag Wiley, 2005.

The preparation according to the process described here produces compounds of the formula (I) and their salts in the form of substance collections which are called libraries. The present invention also provides libraries which comprise at least two compounds of the formula (I) and/or their salts.

The compounds of the formula (I) according to the invention (and/or their salts), hereinbelow also referred to together as “compounds according to the invention”, “compounds (I) according to the invention” or in short as “compounds (I)”, have excellent herbicidal efficacy against a broad spectrum of economically important monocotyledonous and dicotyledonous annual harmful plants. The active compounds act efficiently even on perennial harmful plants which produce shoots from rhizomes, root stocks and other perennial organs and which are difficult to control.

The present invention therefore also relates to a method for controlling unwanted plants or for regulating the growth of plants, preferably in crops of plants, where one or more compound(s) according to the invention is/are applied to the plants (for example harmful plants such as monocotyledonous or dicotyledonous weeds or undesired crop plants), to the seeds (for example grains, seeds or vegetative propagules such as tubers or shoot parts with buds), to the soil in or on which the plants grow (for example the soil of cropland or non-cropland) or to the area on which the plants grow (for example the area under cultivation with plants or plant seeds). In this context, the compounds according to the invention can be applied for example pre-sowing (if appropriate also by incorporation into the soil), pre-emergence or post-emergence. Specific examples may be mentioned of some representatives of the monocotyledonous and dicotyledonous weed flora which can be controlled by the compounds according to the invention, without the enumeration being restricted to certain species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

If the compounds according to the invention are applied to the soil surface before germination, the weed seedlings are either prevented completely from emerging or else the weeds grow until they have reached the cotyledon stage, but then their growth stops, and, eventually, after three to four weeks have elapsed, they die completely.

If the active compounds are applied post-emergence to the green parts of the plants, growth stops after the treatment, and the harmful plants remain at the growth stage of the point of time of application, or they die completely after a certain time, so that in this manner competition by the weeds, which is harmful to the crop plants, is eliminated very early and in a sustained manner.

Although the compounds according to the invention display an outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, in particular Zea and Triticum, are damaged only to an insignificant extent, or not at all, depending on the structure of the respective compound according to the invention and its application rate. This is why the present compounds are highly suitable for the selective control of unwanted plant growth in plant crops such as agriculturally useful plants or ornamentals.

Moreover, the compounds according to the invention (depending on their respective structure and the application rate applied) have outstanding growth-regulatory properties in crop plants. They engage in the plant's metabolism in a regulatory fashion and can therefore be employed for the influencing, in a targeted manner, of plant constituents and for facilitating harvesting, such as, for example, by triggering desiccation and stunted growth. Moreover, they are also suitable for generally controlling and inhibiting unwanted vegetative growth without destroying the plants in the process. Inhibiting the vegetative growth plays an important role in many monocotyledonous and dicotyledonous crops since for example lodging can be reduced, or prevented completely, hereby.

By virtue of their herbicidal and plant-growth-regulatory properties, the active compounds can also be employed for controlling harmful plants in crops of genetically modified plants or plants modified by conventional mutagenesis. In general, the transgenic plants are distinguished by especially advantageous properties, for example by resistances to certain pesticides, mainly certain herbicides, resistances to plant diseases or causative organisms of plant diseases, such as certain insects or microorganisms such as fungi, bacteria or viruses. Other specific characteristics relate, for example, to the harvested material with regard to quantity, quality, storability, composition and specific constituents. Thus, transgenic plants are known whose starch content is increased, or whose starch quality is altered, or those where the harvested material has a different fatty acid composition.

It is preferred with a view to trangenic crops to use the compounds according to the invention and/or their salts in economically important transgenic crops of useful plants and ornamentals, for example of cereals such as wheat, barley, rye, oats, millet, rice and corn or else crops of sugar beet, cotton, soybean, oilseed rape, potato, tomato, peas and other vegetables.

It is preferred to employ the compounds according to the invention as herbicides in crops of useful plants which are resistant, or have been made resistant by recombinant means, to the phytotoxic effects of the herbicides.

By virtue of their herbicidal and plant-growth-regulatory properties, the active compounds can also be employed for controlling harmful plants in crops of known genetically modified plants or genetically modified plants still to be developed. In general, the transgenic plants are distinguished by especially advantageous properties, for example by resistances to certain pesticides, mainly certain herbicides, resistances to plant diseases or causative organisms of plant diseases, such as certain insects or microorganisms such as fungi, bacteria or viruses. Other specific characteristics relate, for example, to the harvested material with regard to quantity, quality, storability, composition and specific constituents. Thus, transgenic plants are known whose starch content is increased, or whose starch quality is altered, or those where the harvested material has a different fatty acid composition. Other particular properties may be tolerance or resistance to abiotic stressors, for example heat, low temperatures, drought, salinity and ultraviolet radiation.

It is preferred to use the compounds of the formula (I) according to the invention or salts thereof in economically important transgenic crops of useful plants and ornamental plants, for example of cereals such as wheat, barley, rye, oats, millet, rice, cassaya and corn or else crops of sugar beet, cotton, soybean, oilseed rape, potato, tomato, peas and other vegetables.

It is preferred to employ the compounds of the formula (I) as herbicides in crops of useful plants which are resistant, or have been made resistant by recombinant means, to the phytotoxic effects of the herbicides.

Conventional methods of generating novel plants which have modified properties in comparison to plants occurring to date consist, for example, in traditional breeding methods and the generation of mutants. Alternatively, novel plants with altered properties can be generated with the aid of recombinant methods (see, for example, EP-A-0221044, EP-A-0131624). For example, the following have been described in several cases:
the modification, by recombinant technology, of crop plants with the aim of modifying the starch synthesized in the plants (for example WO 92/11376, WO 92/14827, WO 91/19806),
transgenic crop plants which are resistant to certain herbicides of the glufosinate type (cf., for example, EP-A-0242236, EP-A-242246) or of the glyphosate type (WO 92/00377) or of the sulfonylurea type (EP-A-0257993, U.S. Pat. No. 5,013,659),
transgenic crop plants, for example cotton, with the capability of producing Bacillus thuringiensis toxins (Bt toxins), which make the plants resistant to certain pests (EP-A-0142924, EP-A-0193259),
transgenic crop plants with a modified fatty acid composition (WO 91/13972),
genetically modified crop plants with novel constituents or secondary metabolites, for example novel phytoalexins, which bring about an increased disease resistance (EPA 309862, EPA0464461),
genetically modified plants with reduced photorespiration which feature higher yields and higher stress tolerance (EPA 0305398),
transgenic crop plants which produce pharmaceutically or diagnostically important proteins (“molecular pharming”),
transgenic crop plants which are distinguished by higher yields or better quality,
transgenic crop plants which are distinguished by a combination, for example of the abovementioned novel properties (“gene stacking”).

A large number of molecular-biological techniques by means of which novel transgenic plants with modified properties can be generated are known in principle; see, for example, I. Potrykus and G. Spangenberg (eds.) Gene Transfer to Plants, Springer Lab Manual (1995), Springer Verlag Berlin, Heidelberg or Christou, “Trends in Plant Science” 1 (1996) 423-431.

To carry out such recombinant manipulations, nucleic acid molecules which allow mutagenesis or a sequence change by recombination of DNA sequences can be introduced into plasmids. For example, base substitutions can be carried out, part-sequences can be removed, or natural or synthetic sequences may be added with the aid of standard methods. To link the DNA fragments with one another, it is possible to add adapters or linkers to the fragments; see, for example, Sambrook et al., 1989, Molecular Cloning, A Laboratory Manual, 2nd ed., Cold Spring Harbor Laboratory Press, Cold Spring Harbor, N.Y.; or Winnacker “Gene and Klone”, VCH Weinheim 2nd ed., 1996.

For example, the generation of plant cells with a reduced activity of a gene product can be achieved by expressing at least one corresponding antisense RNA, a sense RNA for achieving a cosuppression effect or by expressing at least one suitably constructed ribozyme which specifically cleaves transcripts of the above-mentioned gene product.

To this end, it is possible to use DNA molecules which encompass the entire coding sequence of a gene product inclusive of any flanking sequences which may be present, and also DNA molecules which only encompass portions of the coding sequence, it being necessary for these portions to be long enough to have an antisense effect in the cells. The use of DNA sequences which have a high degree of homology to the coding sequences of a gene product, but are not completely identical to them, is also possible.

When expressing nucleic acid molecules in plants, the protein synthesized can be localized in any desired compartment of the plant cell. However, to achieve localization in a particular compartment, it is possible, for example, to link the coding region with DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants can be plants of any desired plant species, i.e. not only monocotyledonous, but also dicotyledonous, plants.

Thus, transgenic plants can be obtained whose properties are altered by overexpression, suppression or inhibition of homologous (=natural) genes or gene sequences or the expression of heterologous (=foreign) genes or gene sequences.

It is preferred to employ the compounds (I) according to the invention in transgenic crops which are resistant to growth regulators such as, for example, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, glyphosate, glufosinate or benzoylisoxazoles and analogous active substances.

When the active compounds according to the invention are used in transgenic crops, effects are frequently observed—in addition to the effects on harmful plants which can be observed in other crops—which are specific for the application in the transgenic crop in question, for example a modified or specifically widened spectrum of weeds which can be controlled, modified application rates which may be employed for application, preferably good combinability with the herbicides to which the transgenic crop is resistant, and an effect on growth and yield of the transgenic crop plants.

The invention therefore also relates to the use of the compounds of the formula (I) according to the invention and/or their salts as herbicides for controlling harmful plants in transgenic crop plants.

The use according to the invention for the control of harmful plants or for growth regulation of plants also includes the case in which the active compound of the formula (I) or its salt is not formed from a precursor substance (“prodrug”) until after application on the plant, in the plant or in the soil.

The compounds (I) according to the invention can be used in the form of wettable powders, emulsifiable concentrates, sprayable solutions, dusting products or granules in the customary formulations. The invention therefore also provides herbicidal and plant growth-regulating compositions which comprise compounds of the formula (I) and/or salts thereof.

The compounds of the formula (I) and/or salts thereof can be formulated in various ways according to which biological and/or physicochemical parameters are required. Possible formulations include, for example: wettable powders (WP), water-soluble powders (SP), water-soluble concentrates, emulsifiable concentrates (EC), emulsions (EW) such as oil-in-water and water-in-oil emulsions, sprayable solutions, suspension concentrates (SC), oil- or water-based dispersions, oil-miscible solutions, capsule suspensions (CS), dusting products (DP), seed-dressing products, granules for scattering and soil application, granules (GR) in the form of microgranules, spray granules, coated granules and adsorption granules, water-dispersible granules (WG), water-soluble granules (SG), ULV formulations, microcapsules and waxes.

These individual types of formulation are known in principle and are described, for example, in: Winnacker-Küchler, “Chemische Technologie” [Chemical technology], Volume 7, C. Hanser Verlag Munich, 4th Ed. 1986; Wade van Valkenburg, “Pesticide Formulations”, Marcel Dekker, N.Y., 1973; K. Martens, “Spray Drying” Handbook, 3rd Ed. 1979, G. Goodwin Ltd. London.

The necessary formulation assistants, such as inert materials, surfactants, solvents and further additives, are likewise known and are described, for example, in: Watkins, “Handbook of Insecticide Dust Diluents and Carriers”, 2nd Ed., Darland Books, Caldwell N.J.; H. v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [Interface-active ethylene oxide adducts], Wiss. Verlagsgesellschaft, Stuttgart 1976; Winnacker-Küchler, “Chemische Technologie”, Volume 7, C. Hanser Verlag Munich, 4th Ed. 1986.

Wettable powders are preparations which can be dispersed uniformly in water and, as well as the active compound, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurinate. To prepare the wettable powders, the herbicidally active compounds are ground finely, for example in customary apparatuses such as hammer mills, blower mills and air-jet mills, and simultaneously or subsequently mixed with the formulation assistants.

Emulsifiable concentrates are prepared by dissolving the active compound in an organic solvent, for example butanol, cyclohexanone, dimethylformamide, xylene or else relatively high-boiling aromatics or hydrocarbons or mixtures of the organic solvents with addition of one or more surfactants of the ionic and/or nonionic type (emulsifiers). The emulsifiers used may, for example, be: alkylarylsulfonic calcium salts, such as

calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, such as, for example, sorbitan fatty acid esters, or polyoxyethylene sorbitan esters, such as, for example, polyoxyethylene sorbitan fatty acid esters.

Dusts are obtained by grinding the active compound with finely distributed solid substances, for example talc, natural clays, such as kaolin, bentonite and pyrophyllite, or diatomaceous earth.

Suspension concentrates may be water- or oil-based. They may be prepared, for example, by wet grinding by means of commercial bead mills and optional addition of surfactants as have, for example, already been listed above for the other formulation types.

Emulsions, e.g. oil-in-water emulsions (EW), can be prepared, for example, by means of stirrers, colloid mills and/or static mixers using aqueous organic solvents and if appropriate surfactants, as have for example already been listed above in connection with the other types of formulation.

Granules can be prepared either by spraying the active compound onto granulated inert material capable of adsorption or by applying active compound concentrates to the surface of carrier substances, such as sand, kaolinites or granulated inert material, by means of adhesives, for example polyvinyl alcohol, sodium polyacrylate or mineral oils. Suitable active compounds can also be granulated in the manner customary for the preparation of fertilizer granules—if desired as a mixture with fertilizers.

Water-dispersible granules are prepared generally by the customary processes such as spray-drying, fluidized bed granulation, pan granulation, mixing with high-speed mixers and extrusion without solid inert material.

For the preparation of pan, fluidized bed, extruder and spray granules, see, for example, processes in “Spray-Drying Handbook” 3rd ed. 1979, G. Goodwin Ltd., London; J. E. Browning, “Agglomeration”, Chemical and Engineering 1967, pages 147 ff; “Perry's Chemical Engineer's Handbook”, 5th Ed., McGraw-Hill, New York 1973, p. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical formulations comprise generally from 0.1 to 99% by weight, in particular from 0.1 to 95% by weight, of active compound of the formula (I) and/or salts thereof.

In wettable powders, the active compound concentration is, for example, from about 10 to 90% by weight, the remainder to 100% by weight consisting of customary formulation components. In the case of emulsifiable concentrates, the active compound concentration can be from about 1 to 90, preferably from 5 to 80, % by weight. Dust-type formulations contain from 1 to 30% by weight of active compound, preferably usually from 5 to 20% by weight of active compound; sprayable solutions contain from about 0.05 to 80% by weight, preferably from 2 to 50% by weight of active compound. In the case of water-dispersible granules, the active compound content depends partially on whether the active compound is present in liquid or solid form and on which granulation auxiliaries, fillers, etc., are used. In the water-dispersible granules, the content of active compound is, for example, between 1 and 95% by weight, preferably between 10 and 80% by weight.

In addition, the active compound formulations mentioned optionally comprise the respective customary adhesives, wetting agents, dispersants, emulsifiers, penetrants, preservatives, antifreeze agents and solvents, fillers, carriers and dyes, defoamers, evaporation inhibitors and agents which influence the pH and the viscosity. Examples of formulation auxiliaries are described inter alia in “Chemistry and Technology of Agrochemical Formulations”, ed. D. A. Knowles, Kluwer Academic Publishers (1998).

The compounds of the formula (I) or salts thereof can be employed as such or in the form of their preparations (formulations) combined with other pesticidally active compounds, such as, for example, insecticides, acaricides, nematicides, herbicides, fungicides, safeners, fertilizers and/or growth regulators, for example as finished formulation or as tank mix. The combination formulations can be prepared on the basis of the abovementioned formulations, while taking account of the physical properties and stabilities of the active ingredients to be combined.

Active compounds which can be employed in combination with the compounds according to the invention in mixed formulations or in the tank mix are, for example, known active compounds which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as are described in, for example, Weed Research 26 (1986) 441-445 or “The Pesticide Manual”, 14th edition, The British Crop Protection Council and the Royal Soc. of Chemistry, 2003 and the literature cited therein. Known herbicides or plant growth regulators which can be combined with the compounds according to the invention are, for example, the following active compounds (the compounds are either designated by the common name according to the International Organization for Standardization (ISO) or by the chemical name, or by the code number) and always comprise all use forms such as acids, salts, esters and isomers such as stereoisomers and optical isomers. Here, by way of example, one and in some cases a plurality of use forms are mentioned: acetochlor, acibenzolar, acibenzolar-S-methyl, acifluorfen, acifluorfen-sodium, aclonifen, alachlor, allidochlor, alloxydim, alloxydim-sodium, ametryne, amicarbazone, amidochlor, amidosulfuron, aminocyclopyrachlor, aminopyralid, amitrole, ammonium sulfamate, ancymidol, anilofos, asulam, atrazine, azafenidin, azimsulfuron, aziprotryne, BAH-043, BAS-140H, BAS-693H, BAS-714H, BAS-762H, BAS-776H, BAS-800H, beflubutamid, benazolin, benazolin-ethyl, bencarbazone, benfluralin, benfuresate, bensulide, bensulfuron-methyl, bentazone, benzfendizone, benzobicyclon, benzofenap, benzofluor, benzoylprop, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbetamide, carfentrazone, carfentrazone-ethyl, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat-chloride, chlornitrofen, chlorophthalim, chlorthal-dimethyl, chlorotoluron, chlorsulfuron, cinidon, cinidon-ethyl, cinmethylin, cinosulfuron, clethodim, clodinafop, clodinafop-propargyl, clofencet, clomazone, clomeprop, cloprop, clopyralid, cloransulam, cloransulam-methyl, cumyluron, cyanamide, cyanazine, cyclanilide, cycloate, cyclosulfamuron, cycloxydim, cycluron, cyhalofop, cyhalofop-butyl, cyperquat, cyprazine, cyprazole, 2,4-D, 2,4-DB, daimuron/dymron, dalapon, daminozide, dazomet, n-decanol, desmedipham, desmetryn, detosyl-pyrazolate (DTP), diallate, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dimefuron, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, dipropetryn, diquat, diquat-dibromide, dithiopyr, diuron, DNOC, eglinazine-ethyl, endothal, EPTC, esprocarb, ethalfluralin, ethametsulfuron, ethametsulfuron-methyl, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, fenoprop, fenoxaprop, fenoxaprop-P, fenoxaprop-ethyl, fenoxaprop-P-ethyl, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, fluorochloridone, fluoroxypyr, fluoroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, L-glufosinate (glufosinate-P), L-glufosinate-ammonium (glufosinate-P-ammonium), glufosinate-P-sodium, glufosinate-ammonium, glyphosate, glyphosate-isopropylammonium, H-9201, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HNPC-9908, HW-02, imazamethabenz, imazamethabenz-methyl, imazamox, imazamox-ammonium imazapic, imazapyr, imazaquin, imazethapyr, imazethapyr-ammonium, imazosulfuron, inabenfide, indanofan, indaziflam, indoleacetic acid (IAA), 4-indol-3-ylbutyric acid (IBA), iodosulfuron, iodosulfuron-methyl-sodium, ioxynil, ipfencarbazone isocarbamid, isopropalin, isoproturon, isouron, isoxaben, isoxachlortole, isoxaflutole, isoxapyrifop, KUH-043, KUH-071, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat-chloride, mesosulfuron, mesosulfuron-methyl, mesotrione, methabenzthiazuron, metam, metamifop, metamitron, metazachlor, methazole, methiozolin, methoxyphenone, methyldymron, 1-methylcyclopropene, methyl isothiocyanate, metobenzuron, metobenzuron, metobromuron, metolachlor, S-metolachlor, metosulam, metoxuron, metribuzin, metsulfuron, metsulfuron-methyl, molinate, monalide, monocarbamide, monocarbamide dihydrogen sulfate, monolinuron, monosulfuron, monosulfuron-ester, monuron, MT 128, MT-5950, i.e. N-[3-chloro-4-(1-methylethyl)phenyl]-2-methylpentanamide, NGGC-011, naproanilide, napropamide, naptalam, NC-620, NC-310, i.e. 4-(2,4-dichlorobenzoyl)-1-methyl-5-benzyloxypyrazole, neburon, nicosulfuron, nipyraclofen, nitralin, nitrofen, nitrophenolat-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazole, paraquat, paraquat dichloride, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazol, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenzpropyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, secbumeton, sethoxydim, siduron, simazine, simetryn, SN-106279, sulcotrione, sulf-allate (CDEC), sulfentrazone, sulfometuron, sulfometuron-methyl, sulfosate (glyphosate-trimesium), sulfosulfuron, SYN-449, SYN-523, SYP-249, SYP-298, SYP-300, tebutam, tebuthiuron, tecnazene, tefuryltrione, tembotrione, tepraloxydim, terbacil, terbucarb, terbuchlor, terbumeton, terbuthylazine, terbutryne, thenylchlor, thiafluamide, thiazafluoron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, triallate, triasulfuron, triaziflam, triazofenamide, tribenuron, tribenuron-methyl, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0166, ZJ-0270, ZJ-0543, ZJ-0862 and also the following compounds

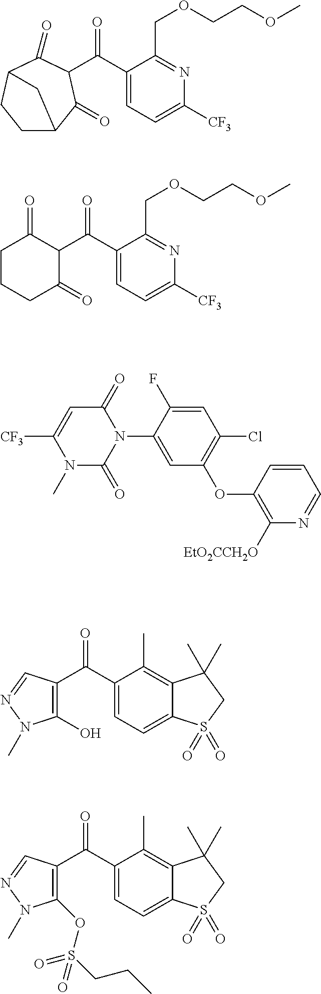

Of particular interest is the selective control of harmful plants in crops of useful plants and ornamentals. Although the compounds (I) according to the invention have already demonstrated very good to adequate selectivity in a large number of crops, in principle, in some crops and in particular also in the case of mixtures with other, less selective herbicides, phytotoxicities on the crop plants may occur. In this connection, combinations of compounds (I) according to the invention are of particular interest which comprise the compounds (I) or their combinations with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugar beet, sugar cane, oilseed rape, cotton and soybeans, preferably cereals. The following groups of compounds are suitable, for example, as safeners for the compounds (I) and their combinations with further pesticides:

A) Compounds of the formula (S-I)

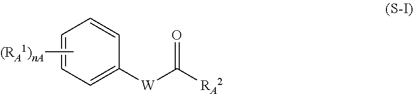

where the symbols and indices have the following meanings:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group consisting of partially unsaturated or aromatic five-membered heterocycles having 1 to 3 ring heteroatoms of the type N or O, where at least one nitrogen atom and at most one oxygen atom is present in the ring, preferably a radical from the group consisting of (W_{A}¹) to (W_{A}⁴),

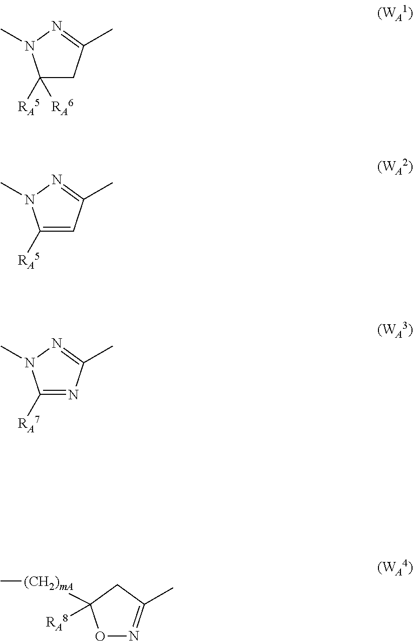

m_{A} is 0 or 1;
R_{A}² is OR_{A}³, SR_{A}³ or NR_{A}³R_{A}⁴ or a saturated
   or unsaturated 3- to 7-membered heterocycle having at least one nitrogen atom and up to 3 heteroatoms, preferably from the group consisting of O and S, which is attached via the nitrogen atom to the carbonyl group in (S-I) and which is unsubstituted or substituted by radicals selected from the group consisting of (C₁-C₄)-alkyl, (C₁-C₄)-alkoxy and optionally substituted phenyl, preferably a radical of the formula OR_{A}³, NHR_{A}⁴ or N(CH₃)₂, in particular of the formula OR_{A}³;
R_{A}³ is hydrogen or an unsubstituted or substituted aliphatic hydrocarbon radical having preferably a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl, (C₁-C₄)-alkoxy-(C₁-C₈)-alkyl, cyano or COOR_{A}⁹ where R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are identical or different and are hydrogen, (C₁-C₈)-alkyl, (C₁-C₈)-haloalkyl, (C₃-C₁₂)-cycloalkyl or substituted or unsubstituted phenyl;
   preferably:
a) compounds of the type of the dichlorophenylpyrazoline-3-carboxylic acid, preferably compounds such as ethyl 1-(2,4-dichlorophenyl)-5-(ethoxycarbonyl)-5-methyl-2-pyrazoline-3-carboxylate (S1-1) (“mefenpyr-diethyl”, see Pestic. Man.), and related compounds, as described in WO 91/07874;
b) derivatives of dichlorophenylpyrazolecarboxylic acid, preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4), ethyl 1-(2,4-dichlorophenyl)-5-phenylpyrazole-3-carboxylate (S1-5) and related compounds, as described in EP-A-333 131 and EP-A-269 806;
c) compounds of the type of the triazolecarboxylic acids, preferably compounds such as fenchlorazole(-ethyl ester), i.e. ethyl 1-(2,4-dichlorophenyl)-5-trichloromethyl-(1H)-1,2,4-triazole-3-carboxylate (S1-6), and related compounds, as described in EP-A-174 562 and EP-A-346 620;
d) compounds of the type of the 5-benzyl- or 5-phenyl-2-isoxazoline-3-carboxylic acid or the 5,5-diphenyl-2-isoxazoline-3-carboxylic acid, preferably compounds such as ethyl 5-(2,4-dichlorobenzyl)-2-isoxazoline-3-carboxylate (S1-7) or ethyl 5-phenyl-2-isoxazoline-3-carboxylate (S1-8) and related compounds, as described in WO 91/08202, or ethyl 5,5-diphenyl-2-isoxazolinecarboxylate (S1-9) (“isoxadifen-ethyl”) or n-propyl 5,5-diphenyl-2-isoxazolinecarboxylate (S1-10) or ethyl 5-(4-fluorophenyl)-5-phenyl-2-isoxazoline-3-carboxylate (S1-11), as described in the patent application WO-A-95/07897.
   B) Quinoline derivatives of the formula (S-II)

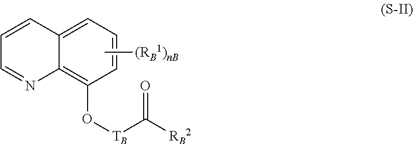

where the symbols and indices have the following meanings:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² OR_{B}³, SR_{B}³ or NR_{B}³R_{B}⁴ or a saturated
   or unsaturated 3- to 7-membered heterocycle having at least one nitrogen atom and up to 3 heteroatoms, preferably from the group consisting of O and S, which is attached via the nitrogen atom to the carbonyl group in (S-II) and is unsubstituted or substituted by radicals selected from the group consisting of (C₁-C₄)-alkyl, (C₁-C₄)-alkoxy or optionally substituted phenyl, preferably a radical of the formula OR_{B}³, NHR_{B}⁴ or N(CH₃)₂, in particular of the formula OR_{B}³;
R_{B}³ is hydrogen or an unsubstituted or substituted aliphatic hydrocarbon radical having preferably a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;
   preferably:
a) compounds of the type of the 8-quinolinoxyacetic acid (S2), preferably 1-methylhexyl (5-chloro-8-quinolinoxy)acetate (common name “cloquintocet-mexyl” (S2-1) (see Pestic. Man.),
   1,3-dimethyl-but-1-yl (5-chloro-8-quinolinoxy)acetate (S2-2),
   4-allyloxybutyl (5-chloro-8-quinolinoxy)acetate (S2-3),
   1-allyloxyprop-2-yl (5-chloro-8-quinolinoxy)acetate (S2-4),
   ethyl (5-chloro-8-quinolinoxy)acetate (S2-5),
   methyl (5-chloro-8-quinolinoxy)acetate (S2-6),
   allyl (5-chloro-8-quinolinoxy)acetate (S2-7),
   2-(2-propylideneiminoxy)-1-ethyl (5-chloro-8-quinolinoxy)acetate (S2-8), t-oxo-prop-1-yl (5-chloro-8-quinolinoxy)acetate (S2-9) and related compounds, as described in EP-A-86 750, EP-A-94 349 and EP-A-191 736 or EP-A-0 492 366, and also their hydrates and salts, as described in WO-A-2002/034048.
b) Compounds of the type of the (5-chloro-8-quinolinoxy)malonic acid, preferably compounds such as diethyl (5-chloro-8-quinolinoxy)malonate, diallyl (5-chloro-8-quinolinoxy)malonate, methyl ethyl(5-chloro-8-quinolinoxy)malonate and related compounds, as described in EP-A-0 582 198.
   C) Compounds of the formula (S-III)

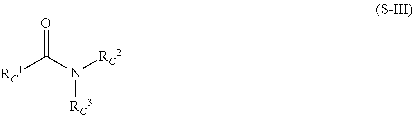

where the symbols and indices have the following meanings:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are identical or different and are hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;
   preferably:
   active compounds of the type of the dichloroacetamides which are frequently used as pre-emergence safener (soil-acting safeners), such as, for example, “dichlormid” (see Pestic. Man.) (═N,N-diallyl-2,2-dichloroacetamide), “R-29148” (=3-dichloroacetyl-2,2,5-trimethyl-1,3-oxazolidine from Stauffer), “R-28725” (=3-dichloroacetyl-2,2,-dimethyl-1,3-oxazolidine from Stauffer), “benoxacor” (see Pestic. Man.) (=4-dichloroacetyl-3,4-dihydro-3-methyl-2H-1,4-benzoxazine),
   “PPG-1292” (═N-allyl-N-[(1,3-dioxolan-2-yl)methyl]dichloroacetamide from PPG Industries),
   “DKA-24” (═N-allyl-N—[(allylaminocarbonyl)methyl]dichloroacetamide from Sagro-Chem),
   “AD-67” or “MON 4660” (=3-dichloroacetyl-1-oxa-3-aza-spiro[4,5]decane from Nitrokemia or Monsanto),
   “TI-35” (=1-dichloroacetylazepane from TRI-Chemical RT) “diclonon” (dicyclonone) or “BAS145138” or “LAB145138” (=3-dichloroacetyl-2,5,5-trimethyl-1,3-diazabicyclo[4.3.0]nonane from BASF) and
   “furilazole” or “MON 13900” (see Pestic. Man.) (═(RS)-3-dichloroacetyl-5-(2-furyl)-2,2-dimethyloxazolidine)
      D) N-Acylsulfonamides of the formula (S-IV) and their salts

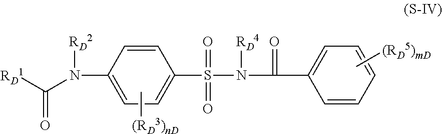

in which
R_{D}¹ is hydrogen, a hydrocarbon radical, a hydrocarbonoxy radical, a hydrocarbonthio radical or a heterocyclyl radical which is preferably attached via a carbon atom, where each of the 4 last-mentioned radicals is unsubstituted or substituted by one or more identical or different radicals selected from the group consisting of halogen, cyano, nitro, amino, hydroxyl, carboxyl, formyl, carbonamide, sulfonamide and radicals of the formula —Z^{a}—R^{a}, where each hydrocarbon moiety has preferably 1 to 20 carbon atoms and a carbon-containing radical R_{D}¹ including substituents has preferably 1 to 30 carbon atoms;
R_{D}² is hydrogen or (C₁-C₄)-alkyl, preferably hydrogen, or
R_{D}¹ and R_{D}² together with the group of the formula —CO—N— are the radical of a 3- to 8-membered saturated or unsaturated ring;
R_{D}³ are identical or different radicals selected from the group consisting of halogen, cyano, nitro, amino, hydroxyl, carboxyl, formyl, CONH₂, SO₂NH₂ or a radical of the formula —Z^{b}—R^{b};
R_{D}⁴ is hydrogen or (C₁-C₄)-alkyl, preferably H;
R_{D}⁵ are identical or different radicals selected from the group consisting of halogen, cyano, nitro, amino, hydroxyl, carboxyl, CHO, CONH₂, SO₂NH₂ and a radical of the formula —Z^{c}—R^{c};
R^{a} is a hydrocarbon radical or a heterocyclyl radical, where each of the two last-mentioned radicals is unsubstituted or substituted by one or more identical or different radicals selected from the group consisting of halogen, cyano, nitro, amino, hydroxyl, mono- and di-[(C₁-C₄)-alkyl]amino, or an alkyl radical in which a plurality, preferably 2 or 3, non-adjacent CH₂ groups are in each case replaced by an oxygen atom;
R^{b},R^{c} are identical or different and are a hydrocarbon radical or a heterocyclyl radical, where each of the two last-mentioned radicals is unsubstituted or substituted by one or more identical or different radicals selected from the group consisting of halogen, cyano, nitro, amino, hydroxyl, phosphoryl, halo-(C₁-C₄)-alkoxy, mono- and di-[(C₁-C₄)-alkyl]amino, or an alkyl radical in which a plurality, preferably 2 or 3, non-adjacent CH₂ groups are in each case replaced by an oxygen atom;
Z^{a} is a divalent group of the formula —O—, —S—, —CO—, —CS—, —CO—O—, —CO—S—, —O—CO—, —S—CO—, —SO—, —SO₂—, —NR*—, —CO—NR*—, —NR*—CO—, —SO₂—NR*— or —NR*—SO₂—, where the bond indicated on the right-hand side of the divalent group in question is the bond to the radical R^{a} and where the R* in the 5 last-mentioned radicals independently of one another are each H, (C₁-C₄)-alkyl or halo-(C₁-C₄)-alkyl;

Z^{b},Z^{c} independently of one another are a direct bond or a divalent group of the formula —O—, —S—, —CO—, —CS—, —CO—O—, —CO—S—, —O—CO—, —S—CO—, —SO—, —SO₂—, —NR*—, —SO₂—NR*—, —NR*—SO₂—, —CO—NR*— or —NR*—CO—, where the bond indicated on the right-hand side of the divalent group in question is the bond to the radical R^{b} or R^{c} and where R* in the 5 last-mentioned radicals independently of one another are each H, (C₁-C₄)-alkyl or halo-(C₁-C₄)-alkyl;
n_{D} is an integer from 0 to 4, preferably 0, 1 or 2, particularly preferably 0 or 1, and
m_{D} is an integer from 0 to 5, preferably 0, 1, 2 or 3, in particular 0, 1 or 2;
   E) acylsulfamoylbenzamides of the formula (S-V), if appropriate also in salt form,

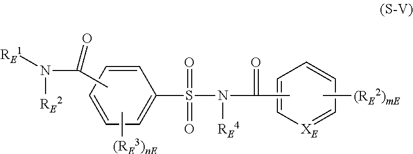

in which
X_{E} is CH or N;
R_{E}¹ is hydrogen, heterocyclyl or a hydrocarbon radical where the two last-mentioned radicals are optionally substituted by one or more identical or different radicals selected from the group consisting of halogen, cyano, nitro, amino, hydroxyl, carboxyl, CHO, CONH₂, SO₂NH₂ and Z^{a}—R^{a};
R_{E}² is hydrogen, hydroxyl, (C₁-C₆)-alkyl, (C₂-C₆)-alkenyl, (C₂-C₆)-alkynyl, (C₁-C₆)-alkoxy, (C₂-C₆)-alkenyloxy, where the five last-mentioned radicals are optionally substituted by one or more identical or different radicals selected from the group consisting of halogen, hydroxyl, (C₁-C₄)-alkyl, (C₁-C₄)-alkoxy and (C₁-C₄)-alkylthio, or
R_{E}¹ and R_{E}² together with the nitrogen atom that carries them are a 3- to 8-membered saturated or unsaturated ring;
R_{E}³ is halogen, cyano, nitro, amino, hydroxyl, carboxyl, CHO, CONH₂, SO₂NH₂ or Z^{b}—R^{b};
R_{E}⁴ is hydrogen, (C₁-C₄)-alkyl, (C₂-C₄)-alkenyl or (C₂-C₄)-alkynyl;
R_{E}⁵ is halogen, cyano, nitro, amino, hydroxyl, carboxyl, phosphoryl, CHO, CONH₂, SO₂NH₂ or Z^{c}—R^{c};
R^{a} is a (C₂-C₂₀)-alkyl radical whose carbon chain is interrupted once or more than once by oxygen atoms, is heterocyclyl or a hydrocarbon radical, where the two last-mentioned radicals are optionally substituted by one or more identical or different radicals selected from the group consisting of halogen, cyano, nitro, amino, hydroxyl, mono- and di-[(C₁-C₄)-alkyl]amino;
R^{b}, R^{c} are identical or different and are a (C₂-C₂₀)-alkyl radical whose carbon chain is interrupted once or more than once by oxygen atoms, are heterocyclyl or a hydrocarbon radical, where the two last-mentioned radicals are optionally substituted by one or more identical or different radicals selected from the group consisting of halogen, cyano, nitro, amino, hydroxyl, phosphoryl, (C₁-C₄)-haloalkoxy, mono- and di-[(C₁-C₄)-alkyl]amino;
Z^{a} is a divalent unit selected from the group consisting of O, S, CO, CS, C(O)O, C(O)S, SO, SO₂, NR^{d}, C(O)NR^{d} and SO₂NR^{d};
Z^{b}, Z^{c} are identical or different and are a direct bond or divalent unit selected from the group consisting of O, S, CO, CS, C(O)O, C(O)S, SO, SO₂, NR^{d}, SO₂NR^{d} and C(O)NR^{d};
R^{d} is hydrogen, (C₁-C₄)-alkyl or (C₁-C₄)-haloalkyl;
n_{E} is an integer from 0 to 4, and
m_{E} if X is CH is an integer from 0 to 5, and if X is N, is an integer from 0 to 4; from among these, preference is given to compounds (also in the form of their salts) of the type of the acylsulfamoylbenzamides, for example of the formula (S-VI) below, which are known, for example, from WO 99/16744,

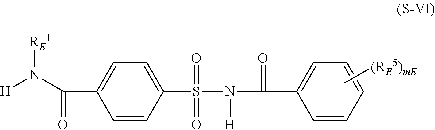

for example those in which
R_{E}¹=cyclopropyl and R_{E}⁵=2-OMe (“cyprosulfamide”, S3-1),
R_{E}¹=cyclopropyl and R_{E}⁵=5-Cl-2-OMe (S3-2),
R_{E}¹=ethyl and R_{E}⁵=2-OMe (S3-3),
R_{E}¹=isopropyl and R_{E}⁵=5-Cl-2-OMe (S3-4), and
R_{E}¹=isopropyl and R_{E}⁵=2-OMe (S3-5);
F) compounds of the type of the N-acylsulfamoylphenylureas of the formula (S-VII), which are known, for example, from EP-A-365484

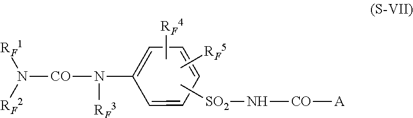

in which
A is a radical from the group consisting of

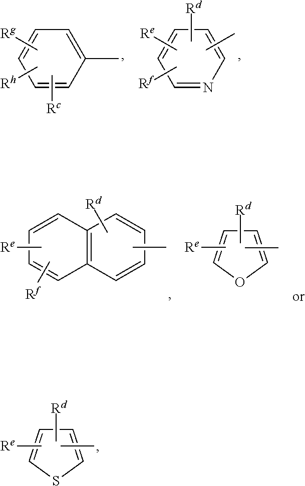

R_{F}¹ and R_{F}² independently of one another represent hydrogen, (C₁-C₈)-alkyl, (C₃-C₈)-cycloalkyl, (C₃-C₆)-alkenyl, (C₃-C₆)-alkynyl,

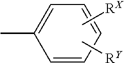

or by (C₁-C₄)-alkoxy or

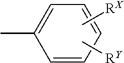

substituted (C₁-C₄)-alkoxy or
R_{F}¹ and R_{F}² together are a C₄-C₆-alkylene bridge or a C₄-C₆-alkylene bridge which is interrupted by oxygen, sulfur, SO, SO₂, NH or —N(C₁-C₄-alkyl)-
R_{F}³ is hydrogen or (C₁-C₄)-alkyl,
R_{F}⁴ and R_{F}⁵ independently of one another are hydrogen, halogen, cyano, nitro, trifluoromethyl, C₁-C₄-alkyl, C₁-C₄-alkoxy, C₁-C₄-alkylthio, C₁-C₄-alkylsulfinyl, C₁-C₄-alkylsulfonyl, —COOR^{j}, —CONR^{k}R^{m}, —CORⁿ, —SO₂NR^{k}R^{m} or —OSO₂—C₁-C₄-alkyl, or R^{a} and R^{b} together are a (C₃-C₄)-alkylene bridge which may be substituted by halogen or (C₁-C₄)-alkyl, or a (C₃-C₄)-alkenylene bridge which may be substituted by halogen or (C₁-C₄)-alkyl, or a C₄-alkadienylene bridge which may be substituted by halogen or (C₁-C₄)-alkyl, and
R^{g} and R^{h} independently of one another are hydrogen, halogen, C₁-C₄-alkyl, trifluoromethyl, methoxy, methylthio or —COOR^{j}, where
R^{c} is hydrogen, halogen, (C₁-C₄)-alkyl or methoxy,
R^{d} is hydrogen, halogen, nitro, (C₁-C₄)-alkyl, (C₁-C₄)-alkoxy, (C₁-C₄)-alkylthio, (C₁-C₄)-alkylsulfinyl, (C₁-C₄)-alkylsulfonyl, —COOR^{j} or —CONR^{k}R^{m},
R^{e} is hydrogen, halogen, C₁-C₄-alkyl, —COOR^{j}, trifluoromethyl or methoxy, or R^{d} and R^{e} together are a (C₃-C₄)-alkylene bridge,
R^{f} is hydrogen, halogen or (C₁-C₄)-alkyl,
R^{X} and R^{Y} independently of one another are hydrogen, halogen, (C₁-C₄)-alkyl, (C₁-C₄)-alkoxy, (C₁-C₄)-alkylthio, —COOR⁴, trifluoromethyl, nitro or cyano,
R^{j}, R^{k} and R^{m} independently of one another are hydrogen or (C₁-C₄)-alkyl,
R^{k} and R^{m} together are a C₄-C₆-alkylene bridge or a C₄-C₆-alkylene bridge which is interrupted by oxygen, NH or —N(C₁-C₄-alkyl)-, and
Rⁿ is (C₁-C₄)-alkyl, phenyl or phenyl which is substituted by halogen, (C₁-C₄)-alkyl, methoxy, nitro or trifluoromethyl,
   from among these, preference is given to:
   1-[4-(N-2-methoxybenzoylsulfamoyl)phenyl]-3-methylurea,
   1-[4-(N-2-methoxybenzoylsulfamoyl)phenyl]-3,3-dimethylurea,
   1-[4-(N-4,5-dimethylbenzoylsulfamoyl)phenyl]-3-methylurea,
   1-[4-(N-naphthoylsulfamoyl)phenyl]-3,3-dimethylurea, including the stereoisomers and the salts customary in agriculture.
G) active compounds from the class of the hydroxyaromatics and aromatic-aliphatic carboxylic acid derivatives, for example
   ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 1,2-dihydro-2-oxo-6-trifluoromethylpyridine-3-carboxamide, 2-hydroxycinnamic acid, 2,4-dichlorocinnamic acid, as described in WO 2004084631,
   WO 2005015994, WO 2006007981, WO 2005016001;
H) active compounds from the class of the 1,2-dihydroquinoxalin-2-ones, for example
   1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydro-quinoxalin-2-one, as described in WO 2005112630,
I) active compounds which, in addition to a herbicidal action against harmful plants, also have safener action on crop plants such as rice, such as, for example,
   “dimepiperate” or “MY-93” (see Pestic. Man.) (═S-1-methyl-1-phenylethyl piperidine-1-thiocarboxylate), which is known as safener for rice against damage by the herbicide molinate,
   “daimuron” or “SK 23” (see Pestic. Man.) (=1-(1-methyl-1-phenylethyl)-3-p-tolylurea), which is known as safener for rice against damage by the herbicide imazosulfuron,
   “cumyluron”=“JC-940” (=3-(2-chlorophenylmethyl)-1-(1-methyl-1-phenyl-ethyl)urea, see JP-A-60087254), which is known as safener for rice against damage by a number of herbicides,
   “methoxyphenone” or “NK 049” (=3,3′-dimethyl-4-methoxybenzophenone), which is known as safener for rice against damage by a number of herbicides,
   “CSB” (=1-bromo-4-(chloromethylsulfonyl)benzene) (CAS Reg. No. 54091-06-4 from Kumiai), which is known as safener against damage by a number of herbicides in rice,
K) compounds of the formula (S-IX), as described in WO-A-1998/38856

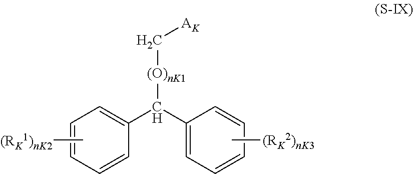

where the symbols and indices have the following meanings:
R_{K}¹, R_{K}² independently of one another are halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;
A_{K} is COOR_{K}³ or COOR_{K}⁴
R_{K}³, R_{K}⁴ independently of one another are hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl or alkylammonium,
n_{K}¹ is 0 or 1 and
n_{K}², n_{K}³ independently of one another are 0, 1 or 2,
   preferably:
   methyl(diphenylmethoxy)acetate (CAS Reg No: 41858-19-9),
L) compounds of the formula (S-X)
   as described in WO A-98/27049

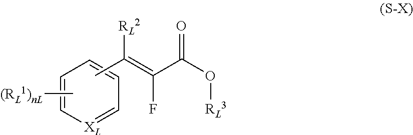

where the symbols and indices have the following meanings:
X_{L} is CH or N,
n_{L} is, in the case that X═N, an integer from 0 to 4 and,
   in the case that X═CH, an integer from 0 to 5,
R_{L}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{L}² is hydrogen or (C₁-C₄)-alkyl,
R_{L}³ is hydrogen, (C₁-C₈)-alkyl, (C₂-C₄)-alkenyl, (C₂-C₄)-alkynyl or aryl, where each of the carbon-containing radicals mentioned above is unsubstituted or substituted by one or more, preferably by up to three, identical or different radicals selected from the group consisting of halogen and alkoxy; or salts thereof,
M) active compounds from the class of the 3-(5-tetrazolylcarbonyl)-2-quinolones, for example
   1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No.: 95855-00-8), as described in WO-A-1999000020,
N) compounds of the formula (S-XI) or (S-XII)
   as described in WO-A-2007023719 and WO-A-2007023764

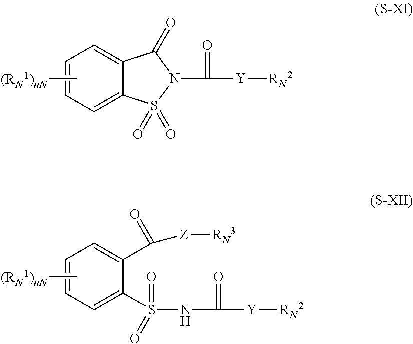

in which
R_{N}¹ is halogen, (C₁-C₄)-alkyl, methoxy, nitro, cyano, CF₃, OCF₃
Y, Z independently of one another are O or S,
n_{N} is an integer from 0 to 4,
R_{N}² is (C₁-C₁₆)-alkyl, (C₂-C₆)-alkenyl, (C₃-C₆)-cycloalkyl, aryl; benzyl, halobenzyl,
R_{N}³ is hydrogen, (C₁-C₆)-alkyl;
O) is one or more compounds from the group consisting of:
   1,8-naphthalic anhydride,
   O,O-diethyl S-2-ethylthioethyl phosphorodithioate (disulfoton),
   4-chlorophenyl methylcarbamate (mephenate),
   O,O-diethyl O-phenyl phosphorothioate (dietholate),
   4-carboxy-3,4-dihydro-2H-1-benzopyran-4-acetic acid (CL-304415, CAS Reg. No.: 31541-57-8),
   2-propenyl 1-oxa-4-azaspiro[4.5]decane-4-carbodithioate (MG-838, CAS Reg. No.: 133993-74-5),
   methyl [(3-oxo-1H-2-benzothiopyran-4(3H)-ylidene)methoxy]acetate (from WO-A-98/13361; CAS Reg. No.: 205121-04-6),
   cyanomethoxyimino(phenyl)acetonitrile (cyometrinil),
   1,3-dioxolan-2-ylmethoxyimino(phenyl)acetonitrile (oxabetrinil),
   4′-chloro-2,2,2-trifluoroacetophenone O-1,3-dioxolan-2-ylmethyloxime (fluxofenim),
   4,6-dichloro-2-phenylpyrimidine (fenclorim),
   benzyl 2-chloro-4-trifluoromethyl-1,3-thiazole-5-carboxylate (flurazole),
   2-dichloromethyl-2-methyl-1,3-dioxolane (MG-191),
   including the stereoisomers, and the salts customary in agriculture.

The weight ratio of herbicide (mixture) to safener generally depends on the herbicide application rate and the effectiveness of the safener in question and may vary within wide limits, for example in the range from 200:1 to 1:200, preferably from 100:1 to 1:100, in particular from 20:1 to 1:20. The safeners may be formulated analogously to the compounds (I) or their mixtures with other herbicides/pesticides and be provided and used as a finished formulation or as a tank mix with the herbicides.

For application, the herbicide or herbicide/safener formulations present in commercial form are, if appropriate, diluted in a customary manner, for example in the case of wettable powders, emulsifiable concentrates, dispersions and water-dispersible granules with water. Preparations in the form of dusts, granules for soil application or granules for broadcasting and sprayable solutions are usually not diluted further with other inert substances prior to application.

The required application rate of the compounds of the formula (I) and/or their salts varies according to the external conditions such as, inter alia, temperature, humidity and the type of herbicide used. It can vary within wide limits. For the application as herbicide for controlling harmful plants, it is, for example, in the range of from 0.001 to 10.0 kg/ha or more of active substance, preferably in the range of from 0.005 to 5 kg/ha, in particular in the range of from 0.01 to 1 kg/ha, of active substance. This applies both to the pre-emergence and the post-emergence application.

When used as plant growth regulator, for example as culm stabilizer for crop plants like those mentioned above, preferably cereal plants, such as wheat, barley, rye, triticale, millet, rice or corn, the application rate is, for example, in the range of from 0.001 to 2 kg/ha or more of active substance, preferably in the range of from 0.005 to 1 kg/ha, in particular in the range of from 10 to 500 g/ha of active substance, very particularly from 20 to 250 g/ha of active substance. This applies both to application by the pre-emergence method and the post-emergence method, the post-emergence treatment generally being preferred.

The application as culm stabilizer may take place at various stages of the growth of the plants. Preferred is, for example, an application after the tillering phase, at the beginning of the longitudinal growth.

As an alternative, application as plant growth regulator is also possible by treating the seed, which includes various techniques for dressing and coating seed. Here, the application rate depends on the particular techniques and can be determined in preliminary tests.

In an exemplary manner, some synthesis examples of compounds of the formula (I) are described below. In the examples, the amounts (including percentages) refer to the weight, unless especially stated otherwise. If, in the context of the description and the examples, the terms “R” and “S” are given for the absolute configuration on a center of chirality of the stereoisomers of the formula (I), this RS nomenclature, follows, unless defined differently, the Cahn-Ingold-Prelog rule.

### (A) SYNTHESIS EXAMPLES

### Example A1

### Preparation of methyl [5-(4-chlorophenyl)-5′-(difluoromethoxy)-1′,3-dimethyl-1′H-1,3′-bipyrazol-4-yl]acetate (see Table 5, Example 5-981)

0.48 g (2.69 mmol) of 5-(difluoromethoxy)-3-hydrazino-1-methyl-1H-pyrazole was added to a solution of 0.50 g (1.86 mmol) of methyl 3-(4-chlorobenzoyl)-4-oxopentanoate in ethanol (5.0 ml). The mixture was heated under reflux for 3 hours and then added to water and extracted with dichloromethane. The combined organic phases were dried over magnesium sulfate, filtered and concentrated. Chromatography gave 0.72 g of product (94% of theory).

NMR (CDCl₃, 300 MHz): 2.36 (s, 3H); 3.38 (s, 2H); 3.68 (s, 3H); 3.70 (s, 3H); 5.50 (s, 1H); 6.40 (t, 1H, J=71 Hz); 7.28 (d, 2H); 7.38 (d, 2H).

### Preparation of methyl 3-(4-chlorobenzoyl)-4-oxopentanoate

A solution of 10 g (51 mmol) of 1-(4-chlorophenyl)butane-1,3-dione (commercially available) dissolved in dimethyl sulfoxide was slowly added dropwise to 2.237 g (56 mmol) of sodium hydride in 200 ml of dimethyl sulfoxide such that the temperature did not exceed 30° C. The mixture was stirred at 20° C. for 30 minutes. 8.558 g (56 mmol) of methyl bromoacetate in a little dimethyl sulfoxide were then slowly added dropwise at 0° C. The mixture was stirred at 20° C. for 4 hours. The reaction mixture was poured into ice-water and extracted with dichloromethane. The organic phase was washed repeatedly with water. Drying of the combined organic phases, removal of the solvent under reduced pressure and chromatography of the residue gave 7.750 g of product (56.7% of theory).

NMR (CDCl₃, 400 MHz): 2.19 (s, 3H); 2.99 (d, 1H); 3.03 (d, 1H); 3.69 (s, 3H); 4.95 (dd, 1H); 7.49 (d, 2H); 7.98 (d, 2H).

### Example A2

### Preparation of [5-(4-chlorophenyl)-5′-(difluoromethoxy)-1′,3-dimethyl-1′H-1,3′-bipyrazol-4-yl]acetic acid (see Table 5, Example 5-162)

1.60 ml of 2-molar aqueous sodium hydroxide solution were added to 0.33 g (0.80 mmol) of methyl [5-(4-chlorophenyl)-5′-(difluoromethoxy)-1′,3-dimethyl-1′H-1,3′-bipyrazol-4-yl]acetate in 8 ml of methanol, and the mixture was stirred at room temperature for one hour. The mixture was concentrated and taken up in 10 ml of water and 15 ml of dichloromethane. The aqueous phase was extracted with dichloromethane, adjusted to pH 3-4 by addition of 2-molar aqueous hydrochloric acid and extracted three times with dichloromethane. The combined organic phases were dried over sodium sulfate, filtered and concentrated. The crude product was dried under high vacuum. This gave 0.14 g of product (44.6% of theory) of a yellow solid of m.p. 112° C.

### Example A3

### Preparation of methyl [5-(4-chlorophenyl)-3-methyl-1-(3-thienyl)-1H-pyrazol-4-yl]acetate (see Table 5, Example 5-876)

0.26 g (1.42 mmol) of copper(II) acetate and 0.15 ml (1.86 mmol) of pyridine were added to 0.25 g (0.94 mmol) of methyl [5-(4-chlorophenyl)-3-methyl-1H-pyrazol-4-yl]acetate in 10 ml of dichloromethane. 0.23 g (1.80 mmol) of 2-thienylboronic acid was then added, and the mixture was stirred at 20° C. for 72 h. The mixture was poured into 1-molar aqueous HCl (10 ml) and extracted with dichloromethane. The combined organic phases were dried over magnesium sulfate, filtered and concentrated. The crude product was purified by preparative HPLC. This gave 0.032 g of product (9.28% of theory).

NMR (CDCl₃, 400 MHz): 2.35 (s, 3H); 3.38 (s, 2H); 3.70 (s, 3H); 6.90 (m, 2H); 7.20 (m, 1H); 7.22 (d, 2H); 7.38 (d, 2H).

### Preparation of methyl [5-(4-chlorophenyl)-3-methyl-1H-pyrazol-4-yl]acetate

2.236 g (45 mmol) of hydrazine hydrate were added to a solution of 10 g (37 mmol) of methyl 3-(4-chlorobenzoyl)-4-oxopentanoate in ethanol (100 ml). The mixture was heated under reflux for 6 hours and then added to water and extracted with dichloromethane. The combined organic phases were dried over magnesium sulfate, filtered and concentrated. This gave 6.9 g of product (63% of theory); NMR (CDCl₃, 400 MHz): 2.28 (s, 3H); 3.50 (s, 2H); 3.70 (s, 3H); 7.39 (d, 2H); 7.50 (d, 2H).

### Example A4

### Preparation of methyl [5-(4-chlorophenyl)-3-methyl-1-(1-methyl-1H-imidazol-5-yl)-1H-pyrazol-4-yl]acetate (see Table 5, Example 5-1107)

0.50 g (2.41 mmol) of 5-iodo-1-methyl-1H-imidazole, 0.06 g (0.32 mmol) of copper iodide, 0.074 g (0.64 mmol) of (S)-(−)-proline (L-proline) and 0.89 g (6.42 mmol) of potassium carbonate were added to 0.43 g (1.61 mmol) of methyl [5-(4-chlorophenyl)-3-methyl-1H-pyrazol-4-yl]acetate in 5 ml of dimethyl sulfoxide. Under an atmosphere of argon, the reaction mixture was heated at 110° C. for 36 h. The mixture was poured into saturated aqueous ammonium chloride solution and extracted with dichloromethane. The combined organic phases were dried over magnesium sulfate, filtered and concentrated. The crude product was purified by preparative HPLC. This gave 0.005 g of product (0.89% of theory).

NMR (CDCl₃, 400 MHz): 2.32 (s, 3H); 3.34 (s, 3H); 3.43 (s, 2H); 3.74 (s, 3H); 6.93 (s, 1H); 7.16 (d, 2H); 7.32 (d, 2H); 7.39 (s, 1H).

The compounds described in Tables 4 and 5 below are obtained according to or analogously to the examples described above.

The compounds described in Tables 4 and 5 below are obtained according to or analogously to the examples described above. Tables 1 to 3 give abbreviations for specific radicals for R⁵, R¹ and Q which are required for describing the specific examples of compounds (I).

In Tables 1 to 5:
F, Cl, Br, I=fluorine, chlorine, bromine or iodine in accordance with the conventional chemical atomic symbols
Me=methyl
MeO or OMe=methoxy
3,5-Me₂=3,5-dimethyl (e.g. as substitution at the phenyl ring)
4,5-Cl₂=4,5-dichloro (e.g. as substitution at the phenyl ring)
Et=ethyl
Pr=nPr=n-propyl
iPr=isopropyl
iOPr=O-iPr=iPrO=isopropyloxy
cyPr (or else “cPr”)=cyclopropyl
Bu=nBu=n-butyl=but-1-yl
iBu=isobutyl=2-methylprop-1-yl
sBu=sec-Bu
tBu=t-butyl=tertiary butyl=2-methylprop-2-yl
Ph=phenyl
PhO=phenoxy
Ac=COCH₃=acetyl
allyl=prop-2-en-1-yl
COOH=carboxyl
COOEt=ethoxycarbonyl
COOMe=methoxycarbonyl
3,5-(COOMe)₂=3,5-dimethoxycarbonyl
OSO₂Me=—O—S(═O)₂—CH₃, methylsulfonyloxy, methanesulfonate
“(R⁶)ₙ═H”=unsubstituted cycle (n=0)

In addition, the customary chemical symbols and formulae apply, such as, for example, CH₂ for methylene or CF₃ for trifluoromethyl or OH for hydroxyl. Correspondingly, composite meanings are defined as composed of the abbreviations mentioned.

Physical data (“Data”) of the compounds in the tables are, if appropriate, given in the comprehensive preparation examples (see above) or at the end of the tables. Here:
“NMR”=data according to the ¹H-NMR spectrum (¹H nuclear resonance data)
“m.p.”=melting point

TABLE 1
Radicals A (specific examples of the radical R⁵ in formula (I))

| No. | A |
|---|---|
| A1 | quinolin-2-yl |
| A2 | isoquinolin-3-yl |
| A3 | 2-pyrimidinyl |
| A4 | 5-F-pyrimidin-2-yl |
| A5 | 5-Cl-pyrimidin-2-yl |
| A6 | 5-Br-pyrimidin-2-yl |
| A7 | 5-Me-pyrimidin-2-yl |
| A8 | 7-Cl-1,3-benzothiazol-2-yl |
| A9 | 1,3-benzothiazol-2-yl |
| A10 | 5-Me-pyrazin-2-yl |
| A11 | 5-Br-1,3-thiazol-2-yl |
| A12 | 5-Cl-1,3-thiazol-2-yl |
| A13 | 2-thiazolyl (=1,3-thiazol-2-yl) |
| A14 | 4-Me-1,3-thiazol-2-yl |
| A15 | 4-OMe-pyridin-2-yl |
| A16 | 4-Me-pyridin-2-yl |
| A17 | 3-pyridyl |
| A18 | 4-pyridyl |
| A19 | 5-F-pyridin-2-yl |
| A20 | 5-Me-pyridin-2-yl |
| A21 | 5-Cl-pyridin-2-yl |
| A22 | 5-Br-pyridin-2-yl |
| A23 | 3-Me-2-thienyl |
| A24 | 4-Me-2-thienyl |
| A25 | 5-Cl-2-thienyl |
| A26 | 2-pyridyl |
| A27 | 5-Br-2-thienyl |
| A28 | 4-Cl—Ph |
| A29 | 4-F—Ph |
| A30 | 4-Me—Ph |
| A31 | Ph |
| A32 | 3-Cl—Ph |
| A33 | 4-F-pyridin-2-yl |
| A34 | 4-Cl-pyridin-2-yl |
| A35 | 4-Br-pyridin-2-yl |
| A36 | 6-Me-pyridin-3-yl |
| A37 | 6-Cl-pyridin-3-yl |
| A38 | 6-Br-pyridin-3-yl |
| A39 | 3-thienyl |
| A40 | 4-Cl-3-thienyl |
| A41 | 4-Br-3-thienyl |
| A42 | 4-Me-3-thienyl |
| A43 | 4-thiazolyl (=1,3-thiazol-4-yl) |
| A44 | 5-thiazolyl (=1,3-thiazol-5-yl) |
| A45 | 2-Me-1,3-thiazol-4-yl |
| A46 | 2-Me-1,3-thiazol-5-yl |
| A47 | 2-pyrazinyl |
| A48 | 5-Cl-3-thienyl |
| A49 | 5-Br-3-thienyl |
| A50 | 5-Me-3-thienyl |
| A51 | 4-Br—Ph |
| A52 | 3,4-F₂—Ph |
| A53 | 1-Me-pyrazol-3-yl |
| A54 | 5-1-2-thienyl |
| A55 | 3,4-Cl₂—Ph |
| A56 | 3-Cl-4-Me—Ph |
| A57 | 2-thienyl |
| A58 | 5-Me-2-thienyl |
| A59 | 3,5-Cl₂—Ph |
| A60 | 5-Me-1,3-thiazol-2-yl |
| A61 | 5-1-pyridin-2-yl |
| A62 | 5-1-pyrimidin-2-yl |
| A63 | 1,3-benzoxazol-2-yl |

TABLE 2
Radicals J (specific examples of the radical R¹ in formula (I))

| No. | J |
|---|---|
| J1 | H |
| J2 | Me |
| J3 | Et |
| J4 | i-Pr |
| J5 | CH₂Ph |
| J6 | prop-2-yn-1-yl |
| J7 | cyclopropylmethyl |
| J8 | 3,3-dichloro-2-fluoroprop-2-en-1-yl |
| J9 | (1-methylcyclopropyl)methyl |
| J10 | (2,2-dichlorocyclopropyl)methyl |
| J11 | allyl |
| J12 | 3-methylbut-2-en-1-yl |
| J13 | 2-methylprop-2-en-1-yl |
| J14 | cyclobutylmethyl |
| J15 | cyclopentylmethyl |
| J16 | 2-methoxyethyl |
| J17 | tetrahydrofuran-2-ylmethyl |
| J18 | oxetan-3-yl |
| J19 | (3-methyloxetan-3-yl)methyl |
| J20 | 2,2,2-trifluoroethyl |
| J21 | CH₂(4-Cl—Ph) |
| J22 | CH₂(4-OMe—Ph) |
| J23 | Ph |
| J24 | 1-ethyl-5-methyl-1H-pyrazol-4-ylmethyl |
| J25 | difluoromethyl |
| J26 | 4,4,4-trifluorobutyl |
| J27 | acetoxymethyl |
| J28 | 2-ethoxyethyl |
| J29 | 1,3-dioxolan-4-ylmethyl |
| J30 | tetrahydrofuran-3-yl |
| J31 | (1,3-dioxan-2-yl)methyl |
| J32 | oxetan-2-ylmethyl |
| J33 | 3,3,3-trifluoropropyl |
| J34 | 2-chlorophenyl |
| J35 | 2-chloropyridin-3-yl |
| J36 | 3-chloropyridin-2-yl |
| J37 | pentafluoroethyl |
| J38 | isobutyl |
| J39 | sBu |
| J40 | 1-(methoxycarbonyl)eth-1-yl |
| J41 | (2,2-dichlorocyclopropyl)methyl |
| J42 | tBu |
| J43 | 1-methylcyclopropyl |
| J44 | 1-methylcyclobutyl |
| J45 | 2-(oxetan-3-yl)eth-1-yl |
| J46 | (N,N-dimethylaminocarbonyl)methyl |
| J47 | [(t-butoxycarbonyl)oxy]methyl |
| J48 | [(methoxycarbonyl)oxy]methyl |
| J49 | 1-acetoxyeth-1-yl |
| J50 | CHMe(4-Cl—Ph) |
| J51 | CHMePh |
| J52 | 1,1,1-trifluoropropan-2-yl |
| J53 | (1-ethyl-3-methyl-1H-pyrazol-4-yl)methyl |
| J54 | Pr |
| J55 | oxetan-3-ylmethyl |
| J56 | 3-methyloxetan-3-yl |

TABLE 3
Radicals Q (specific examples of the heteroaromatic substituent
in the 1-position of the pyrazole radical of the compounds (I))

| No. | Q |
|---|---|
| Q1 | 1H-pyrrol-3-yl |
| Q2 | 1H-pyrrol-2-yl |
| Q3 | 2-Me—1H-pyrrol-3-yl |
| Q4 | 4-Me—1H-pyrrol-3-yl |
| Q5 | 5-Me—1H-pyrrol-3-yl |
| Q6 | 3-Me—1H-pyrrol-2-yl |
| Q7 | 4-Me—1H-pyrrol-2-yl |
| Q8 | 5-Me—1H-pyrrol-2-yl |
| Q9 | 1-Me—1H-pyrrol-3-yl |
| Q10 | 1,2-Me₂—1H-pyrrol-3-yl |
| Q11 | 1,4-Me₂—1H-pyrrol-3-yl |
| Q12 | 1,5-Me₂—1H-pyrrol-3-yl |
| Q13 | 1-Me—1H-pyrrol-2-yl |
| Q14 | 1,3-Me₂—1H-pyrrol-2-yl |
| Q15 | 1,4-Me₂—1H-pyrrol-2-yl |
| Q16 | 1,5-Me₂—1H-pyrrol-2-yl |
| Q17 | 1,3-oxazol-2-yl |
| Q18 | 4-Me-1,3-oxazol-2-yl |
| Q19 | 5-Me-1,3-oxazol-2-yl |
| Q20 | 1,3-oxazol-4-yl |
| Q21 | 2-Me-1,3-oxazol-4-yl |
| Q22 | 5-Me-1,3-oxazol-4-yl |
| Q23 | 1,3-oxazol-5-yl |
| Q24 | 2-Me-1,3-oxazol-5-yl |
| Q25 | 4-Me-1,3-oxazol-5-yl |
| Q26 | 2-Cl-1,3-oxazol-5-yl |
| Q27 | 4-Cl-1,3-oxazol-5-yl |
| Q28 | 2-F-1,3-oxazol-5-yl |
| Q29 | 4-F-1,3-oxazol-5-yl |
| Q30 | isoxazol-3-yl |
| Q31 | isoxazol-4-yl |
| Q32 | isoxazol-5-yl |
| Q33 | 5-Me-isoxazol-3-yl |
| Q34 | 4-Me-isoxazol-3-yl |
| Q35 | 3-Me-isoxazol-4-yl |
| Q36 | 5-Me-isoxazol-4-yl |
| Q37 | 5-Cl-isoxazol-4-yl |
| Q38 | 5-F-isoxazol-4-yl |
| Q39 | 5-CN-isoxazol-4-yl |
| Q40 | 3-Me-isoxazol-5-yl |
| Q41 | 4-Me-isoxazol-5-yl |
| Q42 | 2-furyl |
| Q43 | 3-Me-furan-2-yl |
| Q44 | 4-Me-furan-2-yl |
| Q45 | 5-Me-furan-2-yl |
| Q46 | 3-furyl |
| Q47 | 2-Me-furan-3-yl |
| Q48 | 4-Me-furan-3-yl |
| Q49 | 4-F-furan-3-yl |
| Q50 | 4-Cl-furan-3-yl |
| Q51 | 5-Me-furan-3-yl |
| Q52 | 2-thienyl |
| Q53 | 3-Me-thien-2-yl |
| Q54 | 4-Me-thien-2-yl |
| Q55 | 5-Me-thien-2-yl |
| Q56 | 3-thienyl |
| Q57 | 2-Me-thien-3-yl |
| Q58 | 2-COOMe-thien-3-yl |
| Q59 | 2-COOH-thien-3-yl |
| Q60 | 4-F-thien-3-yl |
| Q61 | 4-Cl-thien-3-yl |
| Q62 | 4-Me-thien-3-yl |
| Q63 | 5-Me-thien-3-yl |
| Q64 | 1-Me—1H-pyrazol-3-yl |
| Q65 | 1-Me—1H-pyrazol-4-yl |
| Q66 | 1-Me—1H-pyrazol-5-yl |
| Q67 | 1H-pyrazol-3-yl |
| Q68 | 1H-pyrazol-4-yl |
| Q69 | 1H-pyrazol-5-yl |
| Q70 | 1-Me-5-OCF₂H—1H-pyrazol-3-yl |
| Q71 | 1-Me-5-OCF₃—1H-pyrazol-3-yl |
| Q72 | 1-Me-5-F—1H-pyrazol-3-yl |
| Q73 | 1-Me-4-F—1H-pyrazol-3-yl |
| Q74 | 1-Me-5-F—1H-pyrazol-4-yl |
| Q75 | 1-Me-5-Cl—1H-pyrazol-4-yl |
| Q76 | 1,5-Me₂—1H-pyrazol-4-yl |
| Q77 | 1-Me-5-Br—1H-pyrazol-4-yl |
| Q78 | 1-benzyl-1H-pyrazol-4-yl |
| Q79 | 1-(tetrahydropyran-2-yl)-1H-pyrazol-4-yl |
| Q80 | 3-Me-4-COOEt—1H-pyrazol-5-yl |
| Q81 | 1,3-Me₂-4-nitro-1H-pyrazol-5-yl |
| Q82 | 1,4-Me₂—1H-pyrazol-5-yl |
| Q83 | 1-Me-4-F—1H-pyrazol-5-yl |
| Q84 | 1-Me-4-Cl—1H-pyrazol-5-yl |
| Q85 | 1-Me-4-Br—1H-pyrazol-5-yl |
| Q86 | 1-Me-4-CN—1H-pyrazol-5-yl |
| Q87 | 1-Me-3-F—1H-pyrazol-5-yl |
| Q88 | 1-Me-3,4-F₂—1H-pyrazol-5-yl |
| Q89 | 3,4-F₂—1H-pyrazol-5-yl |
| Q90 | 3-F—1H-pyrazol-5-yl |
| Q91 | 1-Me—1H-imidazol-2-yl |
| Q92 | 1-Me-4-F—1H-imidazol-2-yl |
| Q93 | 1-Me-5-F—1H-imidazol-2-yl |
| Q94 | 4-F—1H-imidazol-2-yl |
| Q95 | 5-F—1H-imidazol-2-yl |
| Q96 | 1-Me-4-Cl—1H-imidazol-2-yl |
| Q97 | 1-Me-5-Cl—1H-imidazol-2-yl |
| Q98 | 1-Me—1H-imidazol-4-yl |
| Q99 | 1-Me-2-F—1H-imidazol-4-yl |
| Q100 | 1-Me-5-F—1H-imidazol-4-yl |
| Q101 | 1,2-Me₂—1H-imidazol-4-yl |
| Q102 | 1,5-Me₂—1H-imidazol-4-yl |
| Q103 | 1-Me-2-Cl—1H-imidazol-4-yl |
| Q104 | 1-Me-5-Cl—1H-imidazol-4-yl |
| Q105 | 1-Me-2,5-F₂—1H-imidazol-4-yl |
| Q106 | 1-Me—1H-imidazol-5-yl |
| Q107 | 1-Me-3-F—1H-imidazol-5-yl |
| Q108 | 1-Me-3-Cl—1H-imidazol-5-yl |
| Q109 | 1-Me-3-Br—1H-imidazol-5-yl |
| Q110 | 1,3-Me₂—1H-imidazol-5-yl |
| Q111 | 1-Me-4-F—1H-imidazol-5-yl |
| Q112 | 1-Me-4-Cl—1H-imidazol-5-yl |
| Q113 | 1-Me-4-Br—1H-imidazol-5-yl |
| Q114 | 1,4-Me₂—1H-imidazol-5-yl |
| Q115 | 1-Me-3,4-F₂—1H-imidazol-5-yl |
| Q116 | 1,3,4-Me₃—1H-imidazol-5-yl |
| Q117 | 1-Me-3,4-Cl₂—1H-imidazol-5-yl |
| Q118 | 1-Et—1H-imidazol-5-yl |
| Q119 | 1-benzyl-1H-imidazol-5-yl |
| Q120 | 1-amino-1H-imidazol-5-yl |
| Q121 | 1-COOtBu—1H-imidazol-5-yl |
| Q122 | 1-cPr—1H-imidazol-5-yl |
| Q123 | 1H-imidazol-2-yl |
| Q124 | 1H-imidazol-4-yl |
| Q125 | 1H-imidazol-5-yl |
| Q126 | 1,2,4-oxadiazol-3-yl |
| Q127 | 5-F-1,2,4-oxadiazol-3-yl |
| Q128 | 5-Cl-1,2,4-oxadiazol-3-yl |
| Q129 | 5-Me-1,2,4-oxadiazol-3-yl |
| Q130 | 1,2,4-oxadiazol-5-yl |
| Q131 | 3-F-1,2,4-oxadiazol-5-yl |
| Q132 | 3-Cl-1,2,4-oxadiazol-5-yl |
| Q133 | 3-Me-1,2,4-oxadiazol-5-yl |
| Q134 | 1,2,4-thiadiazol-3-yl |
| Q135 | 5-F-1,2,4-thiadiazol-3-yl |
| Q136 | 5-Cl-1,2,4-thiadiazol-3-yl |
| Q137 | 5-Me-1,2,4-thiadiazol-3-yl |
| Q138 | 1,2,4-thiadiazol-5-yl |
| Q139 | 3-F-1,2,4-thiadiazol-5-yl |
| Q140 | 3-Cl-1,2,4-thiadiazol-5-yl |
| Q141 | 3-Me-1,2,4-thiadiazol-5-yl |
| Q142 | 3-SMe-1,2,4-thiadiazol-5-yl |
| Q143 | 1,3,4-oxadiazol-2-yl |
| Q144 | 5-F-1,3,4-oxadiazol-2-yl |
| Q145 | 5-Cl-1,3,4-oxadiazol-2-yl |
| Q146 | 5-Me-1,3,4-oxadiazol-2-yl |
| Q147 | 1,3,4-thiadiazol-2-yl |
| Q148 | 5-F-1,3,4-thiadiazol-2-yl |
| Q149 | 5-Cl-1,3,4-thiadiazol-2-yl |
| Q150 | 5-Me-1,3,4-thiadiazol-2-yl |
| Q151 | 5-CF₃-1,3,4-thiadiazol-2-yl |
| Q152 | 1H-1,2,3,4-tetrazol-5-yl |
| Q153 | 2H-1,2,3,4-tetrazol-5-yl |
| Q154 | 1-Me—1H-1,2,3,4-tetrazol-5-yl |
| Q155 | 2-Me—2H-1,2,3,4-tetrazol-5-yl |
| Q156 | 1,2,3-thiadiazol-4-yl |
| Q157 | 5-F-1,2,3-thiadiazol-4-yl |
| Q158 | 5-Cl-1,2,3-thiadiazol-4-yl |
| Q159 | 5-Me-1,2,3-thiadiazol-4-yl |
| Q160 | 1,2,3-thiadiazol-5-yl |
| Q161 | 4-F-1,2,3-thiadiazol-5-yl |
| Q162 | 4-Cl-1,2,3-thiadiazol-5-yl |
| Q163 | 4-Cl-1,2,3-thiadiazol-5-yl |
| Q164 | 1,2,5-oxadiazol-3-yl |
| Q165 | 4-F-1,2,5-oxadiazol-3-yl |
| Q166 | 4-Cl-1,2,5-oxadiazol-3-yl |
| Q167 | 4-Me-1,2,5-oxadiazol-3-yl |
| Q168 | 1,2,5-thiadiazol-3-yl |
| Q169 | 4-F-1,2,5-thiadiazol-3-yl |
| Q170 | 4-Cl-1,2,5-thiadiazol-3-yl |
| Q171 | 4-Me-1,2,5-thiadiazol-3-yl |
| Q172 | 1H-1,2,3-triazol-4-yl |
| Q173 | 1-Me—1H-1,2,3-triazol-4-yl |
| Q174 | 1,5-Me₂—1H-1,2,3-triazol-4-yl |
| Q175 | 1-Me-5-F—1H-1,2,3-triazol-4-yl |
| Q176 | 1-Me-5-Cl—1H-1,2,3-triazol-4-yl |
| Q177 | 1H-1,2,3-triazol-5-yl |
| Q178 | 1-Me—1H-1,2,3-triazol-5-yl |
| Q179 | 1,4-Me₂—1H-1,2,3-triazol-5-yl |
| Q180 | 1-Me-4-F—1H-1,2,3-triazol-5-yl |
| Q181 | 1-Me-4-Cl—1H-1,2,3-triazol-5-yl |
| Q182 | 1-Et—1H-1,2,3-triazol-5-yl |
| Q183 | 1-Et-4-Me—1H-1,2,3-triazol-5-yl |
| Q184 | 1-Et-4-F—1H-1,2,3-triazol-5-yl |
| Q185 | 1-Et-4-Cl—1H-1,2,3-triazol-5-yl |
| Q186 | 1-cPr—1H-1,2,3-triazol-5-yl |
| Q187 | 1-cPr-4-Me—1H-1,2,3-triazol-5-yl |
| Q188 | 1-cPr-4-F—1H-1,2,3-triazol-5-yl |
| Q189 | 1-cPr-4-Cl—1H-1,2,3-triazol-5-yl |
| Q190 | 2H-1,2,3-triazol-4-yl |
| Q191 | 2-Me—2H-1,2,3-triazol-4-yl |
| Q192 | 2-Me-5-F—2H-1,2,3-triazol-4-yl |
| Q193 | 2-Me-5-Cl—2H-1,2,3-triazol-4-yl |
| Q194 | 2,5-Me₂—2H-1,2,3-triazol-4-yl |
| Q195 | 5-Me—2H-1,2,3-triazol-4-yl |
| Q196 | 1H-1,2,4-triazol-3-yl |
| Q197 | 1H-1,2,4-triazol-5-yl |
| Q198 | 1-Me—1H-1,2,4-triazol-3-yl |
| Q199 | 1-Me—1H-1,2,4-triazol-5-yl |
| Q200 | 1-Me-5-F—1H-1,2,4-triazol-3-yl |
| Q201 | 1-Me-3-F—1H-1,2,4-triazol-5-yl |
| Q202 | 1-Me-5-Cl—1H-1,2,4-triazol-3-yl |
| Q203 | 1-Me-3-Cl—1H-1,2,4-triazol-5-yl |
| Q204 | 1,3-Me₂—1H-1,2,4-triazol-5-yl |
| Q205 | 1,5-Me₂—1H-1,2,4-triazol-3-yl |
| Q206 | 1-Et—1H-1,2,4-triazol-5-yl |
| Q207 | 1-Et-3-Me—1H-1,2,4-triazol-5-yl |
| Q208 | 1-Et-3-F—1H-1,2,4-triazol-5-yl |
| Q209 | 1-Et-3-Cl—1H-1,2,4-triazol-5-yl |
| Q210 | 1-cPr—1H-1,2,4-triazol-5-yl |
| Q211 | 1-cPr-3-Me—1H-1,2,4-triazol-5-yl |
| Q212 | 1-cPr-3-F—1H-1,2,4-triazol-5-yl |
| Q213 | 1-cPr-3-Cl—1H-1,2,4-triazol-5-yl |
| Q214 | 4H-1,2,4-triazol-3-yl |
| Q215 | 5-F—4H-1,2,4-triazol-3-yl |
| Q216 | 5-Cl—4H-1,2,4-triazol-3-yl |
| Q217 | 5-Me—4H-1,2,4-triazol-3-yl |
| Q218 | 5-iPr—4H-1,2,4-triazol-3-yl |
| Q219 | 5-cPr—4H-1,2,4-triazol-3-yl |
| Q220 | 5-tBu—4H-1,2,4-triazol-3-yl |
| Q221 | 4-Me—4H-1,2,4-triazol-3-yl |
| Q222 | 4,5-Me₂—4H-1,2,4-triazol-3-yl |
| Q223 | 4-Me-5-F—4H-1,2,4-triazol-3-yl |
| Q224 | 4-Me-5-Cl—4H-1,2,4-triazol-3-yl |
| Q225 | 4-Et—4H-1,2,4-triazol-3-yl |
| Q226 | 4-Et-5-Me—4H-1,2,4-triazol-3-yl |
| Q227 | 4-Et-5-F—4H-1,2,4-triazol-3-yl |
| Q228 | 4-Et-5-Cl—4H-1,2,4-triazol-3-yl |
| Q229 | 4-cPr—4H-1,2,4-triazol-3-yl |
| Q230 | 4-cPr-5-Me—4H-1,2,4-triazol-3-yl |
| Q231 | 4-cPr-5-F—4H-1,2,4-triazol-3-yl |
| Q232 | 4-cPr-5-Cl—4H-1,2,4-triazol-3-yl |
| Q233 | 1,2,3-oxadiazol-4-yl |
| Q234 | 5-Me-1,2,3-oxadiazol-4-yl |
| Q235 | 5-Cl-1,2,3-oxadiazol-4-yl |
| Q236 | 5-F-1,2,3-oxadiazol-4-yl |
| Q237 | 1,2,3-oxadiazol-5-yl |
| Q238 | 4-Me-1,2,3-oxadiazol-5-yl |
| Q239 | 4-Cl-1,2,3-oxadiazol-5-yl |
| Q240 | 4-F-1,2,3-oxadiazol-5-yl |
| Q241 | 1,2,3,4-oxatriazol-5-yl |
| Q242 | 1,2,3,4-thiatriazol-5-yl |
| Q243 | 1,2,3,5-oxatriazol-4-yl |
| Q244 | 1,2,3,5-thiatriazol-4-yl |
| Q245 | 1,3-thiazol-2-yl |
| Q246 | 4-F-1,3-thiazol-2-yl |
| Q247 | 4-Cl-1,3-thiazol-2-yl |
| Q248 | 4-Me-1,3-thiazol-2-yl |
| Q249 | 5-F-1,3-thiazol-2-yl |
| Q250 | 5-Cl-1,3-thiazol-2-yl |
| Q251 | 5-Me-1,3-thiazol-2-yl |
| Q252 | 4,5-Me₂-1,3-thiazol-2-yl |
| Q253 | 4-CF₂H-5-Cl-1,3-thiazol-2-yl |
| Q254 | 4-CF₃-5-Cl-1,3-thiazol-2-yl |
| Q255 | 4-CN-5-Cl-1,3-thiazol-2-yl |
| Q256 | 4,5-F₂-1,3-thiazol-2-yl |
| Q257 | 1,3-thiazol-4-yl |
| Q258 | 2-F-1,3-thiazol-4-yl |
| Q259 | 2-Cl-1,3-thiazol-4-yl |
| Q260 | 2-Me-1,3-thiazol-4-yl |
| Q261 | 5-F-1,3-thiazol-4-yl |
| Q262 | 5-Cl-1,3-thiazol-4-yl |
| Q263 | 5-Me-1,3-thiazol-4-yl |
| Q264 | 5-COOMe-thiazol-4-yl |
| Q265 | 5-COOH-1,3-thiazol-4-yl |
| Q266 | 2-F-5-Me-1,3-thiazol-4-yl |
| Q267 | isothiazol-3-yl |
| Q268 | 4-F-isothiazol-3-yl |
| Q269 | 4-Cl-isothiazol-3-yl |
| Q270 | 4-Me-isothiazol-3-yl |
| Q271 | 5-F-isothiazol-3-yl |
| Q272 | 5-Cl-isothiazol-3-yl |
| Q273 | 5-Me-isothiazol-3-yl |
| Q274 | 4,5-Me₂-isothiazol-3-yl |
| Q275 | 4,5-Cl₂-isothiazol-3-yl |

TABLE 4
Compounds of the formula (I-1)
(I-1)

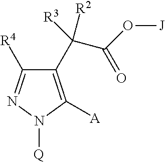

In the table below, for the compounds of the formula (I-1), the
examples of the radicals A, J and Q in the respective column are
referred to by the abbreviations (example numbers) as defined
in Tables 1 to 3 for the radicals A, J and Q, respectively.

| No. | R2 | R3 | R4 | A | J | Q |
|---|---|---|---|---|---|---|
| 4-1 | H | H | H | A1 | J2 | Q23 |
| 4-2 | H | H | Cl | A1 | J2 | Q23 |
| 4-3 | H | H | Br | A1 | J2 | Q23 |
| 4-4 | H | H | CH₂F | A1 | J2 | Q23 |
| 4-5 | H | H | CHF2 | A1 | J2 | Q23 |
| 4-6 | H | H | Et | A1 | J2 | Q23 |
| 4-7 | H | H | Pr | A1 | J2 | Q23 |
| 4-8 | H | H | cPr | A1 | J2 | Q23 |
| 4-9 | H | H | ethynyl | A1 | J2 | Q23 |
| 4-10 | F | H | Me | A1 | J2 | Q23 |
| 4-11 | F | F | Me | A1 | J2 | Q23 |
| 4-12 | Me | H | Me | A1 | J2 | Q23 |
| 4-13 | Me | Me | Me | A1 | J2 | Q23 |
| 4-14 | H | H | H | A2 | J2 | Q23 |
| 4-15 | H | H | Cl | A2 | J2 | Q23 |
| 4-16 | H | H | Br | A2 | J2 | Q23 |
| 4-17 | H | H | CH₂F | A2 | J2 | Q23 |
| 4-18 | H | H | CHF2 | A2 | J2 | Q23 |
| 4-19 | H | H | Et | A2 | J2 | Q23 |
| 4-20 | H | H | Pr | A2 | J2 | Q23 |
| 4-21 | H | H | cPr | A2 | J2 | Q23 |
| 4-22 | H | H | ethynyl | A2 | J2 | Q23 |
| 4-23 | F | H | Me | A2 | J2 | Q23 |
| 4-24 | F | F | Me | A2 | J2 | Q23 |
| 4-25 | Me | H | Me | A2 | J2 | Q23 |
| 4-26 | Me | Me | Me | A2 | J2 | Q23 |
| 4-27 | H | H | H | A6 | J2 | Q23 |
| 4-28 | H | H | Cl | A6 | J2 | Q23 |
| 4-29 | H | H | Br | A6 | J2 | Q23 |
| 4-30 | H | H | CH₂F | A6 | J2 | Q23 |
| 4-31 | H | H | CHF2 | A6 | J2 | Q23 |
| 4-32 | H | H | Et | A6 | J2 | Q23 |
| 4-33 | H | H | Pr | A6 | J2 | Q23 |
| 4-34 | H | H | cPr | A6 | J2 | Q23 |
| 4-35 | H | H | ethynyl | A6 | J2 | Q23 |
| 4-36 | F | H | Me | A6 | J2 | Q23 |
| 4-37 | F | F | Me | A6 | J2 | Q23 |
| 4-38 | Me | H | Me | A6 | J2 | Q23 |
| 4-39 | Me | Me | Me | A6 | J2 | Q23 |
| 4-40 | H | H | H | A9 | J2 | Q23 |
| 4-41 | H | H | Cl | A9 | J2 | Q23 |
| 4-42 | H | H | Br | A9 | J2 | Q23 |
| 4-43 | H | H | CH₂F | A9 | J2 | Q23 |
| 4-44 | H | H | CHF2 | A9 | J2 | Q23 |
| 4-45 | H | H | Et | A9 | J2 | Q23 |
| 4-46 | H | H | Pr | A9 | J2 | Q23 |
| 4-47 | H | H | cPr | A9 | J2 | Q23 |
| 4-48 | H | H | ethynyl | A9 | J2 | Q23 |
| 4-49 | F | H | Me | A9 | J2 | Q23 |
| 4-50 | F | F | Me | A9 | J2 | Q23 |
| 4-51 | Me | H | Me | A9 | J2 | Q23 |
| 4-52 | Me | Me | Me | A9 | J2 | Q23 |
| 4-53 | H | H | H | A11 | J2 | Q23 |
| 4-54 | H | H | Cl | A11 | J2 | Q23 |
| 4-55 | H | H | Br | A11 | J2 | Q23 |
| 4-56 | H | H | CH₂F | A11 | J2 | Q23 |
| 4-57 | H | H | CHF2 | A11 | J2 | Q23 |
| 4-58 | H | H | Et | A11 | J2 | Q23 |
| 4-59 | H | H | Pr | A11 | J2 | Q23 |
| 4-60 | H | H | cPr | A11 | J2 | Q23 |
| 4-61 | H | H | ethynyl | A11 | J2 | Q23 |
| 4-62 | F | H | Me | A11 | J2 | Q23 |
| 4-63 | F | F | Me | A11 | J2 | Q23 |
| 4-64 | Me | H | Me | A11 | J2 | Q23 |
| 4-65 | Me | Me | Me | A11 | J2 | Q23 |
| 4-66 | H | H | H | A12 | J2 | Q23 |
| 4-67 | H | H | Cl | A12 | J2 | Q23 |
| 4-68 | H | H | Br | A12 | J2 | Q23 |
| 4-69 | H | H | CH₂F | A12 | J2 | Q23 |
| 4-70 | H | H | CHF2 | A12 | J2 | Q23 |
| 4-71 | H | H | Et | A12 | J2 | Q23 |
| 4-72 | H | H | Pr | A12 | J2 | Q23 |
| 4-73 | H | H | cPr | A12 | J2 | Q23 |
| 4-74 | H | H | ethynyl | A12 | J2 | Q23 |
| 4-75 | F | H | Me | A12 | J2 | Q23 |
| 4-76 | F | F | Me | A12 | J2 | Q23 |
| 4-77 | Me | H | Me | A12 | J2 | Q23 |
| 4-78 | Me | Me | Me | A12 | J2 | Q23 |
| 4-79 | H | H | H | A19 | J2 | Q23 |
| 4-80 | H | H | Cl | A19 | J2 | Q23 |
| 4-81 | H | H | Br | A19 | J2 | Q23 |
| 4-82 | H | H | CH₂F | A19 | J2 | Q23 |
| 4-83 | H | H | CHF2 | A19 | J2 | Q23 |
| 4-84 | H | H | Et | A19 | J2 | Q23 |
| 4-85 | H | H | Pr | A19 | J2 | Q23 |
| 4-86 | H | H | cPr | A19 | J2 | Q23 |
| 4-87 | H | H | ethynyl | A19 | J2 | Q23 |
| 4-88 | F | H | Me | A19 | J2 | Q23 |
| 4-89 | F | F | Me | A19 | J2 | Q23 |
| 4-90 | Me | H | Me | A19 | J2 | Q23 |
| 4-91 | Me | Me | Me | A19 | J2 | Q23 |
| 4-92 | H | H | H | A20 | J2 | Q23 |
| 4-93 | H | H | Cl | A20 | J2 | Q23 |
| 4-94 | H | H | Br | A20 | J2 | Q23 |
| 4-95 | H | H | CH₂F | A20 | J2 | Q23 |
| 4-96 | H | H | CHF2 | A20 | J2 | Q23 |
| 4-97 | H | H | Et | A20 | J2 | Q23 |
| 4-98 | H | H | Pr | A20 | J2 | Q23 |
| 4-99 | H | H | cPr | A20 | J2 | Q23 |
| 4-100 | H | H | ethynyl | A20 | J2 | Q23 |
| 4-101 | F | H | Me | A20 | J2 | Q23 |
| 4-102 | F | F | Me | A20 | J2 | Q23 |
| 4-103 | Me | H | Me | A20 | J2 | Q23 |
| 4-104 | Me | Me | Me | A20 | J2 | Q23 |
| 4-105 | H | H | H | A21 | J2 | Q23 |
| 4-106 | H | H | Cl | A21 | J2 | Q23 |
| 4-107 | H | H | Br | A21 | J2 | Q23 |
| 4-108 | H | H | CH₂F | A21 | J2 | Q23 |
| 4-109 | H | H | CHF2 | A21 | J2 | Q23 |
| 4-110 | H | H | Et | A21 | J2 | Q23 |
| 4-111 | H | H | Pr | A21 | J2 | Q23 |
| 4-112 | H | H | cPr | A21 | J2 | Q23 |
| 4-113 | H | H | ethynyl | A21 | J2 | Q23 |
| 4-114 | F | H | Me | A21 | J2 | Q23 |
| 4-115 | F | F | Me | A21 | J2 | Q23 |
| 4-116 | Me | H | Me | A21 | J2 | Q23 |
| 4-117 | Me | Me | Me | A21 | J2 | Q23 |
| 4-118 | H | H | H | A22 | J2 | Q23 |
| 4-119 | H | H | Cl | A22 | J2 | Q23 |
| 4-120 | H | H | Br | A22 | J2 | Q23 |
| 4-121 | H | H | CH₂F | A22 | J2 | Q23 |
| 4-122 | H | H | CHF2 | A22 | J2 | Q23 |
| 4-123 | H | H | Et | A22 | J2 | Q23 |
| 4-124 | H | H | Pr | A22 | J2 | Q23 |
| 4-125 | H | H | cPr | A22 | J2 | Q23 |
| 4-126 | H | H | ethynyl | A22 | J2 | Q23 |
| 4-127 | F | H | Me | A22 | J2 | Q23 |
| 4-128 | F | F | Me | A22 | J2 | Q23 |
| 4-129 | Me | H | Me | A22 | J2 | Q23 |
| 4-130 | Me | Me | Me | A22 | J2 | Q23 |
| 4-131 | H | H | H | A26 | J2 | Q23 |
| 4-132 | H | H | Cl | A26 | J2 | Q23 |
| 4-133 | H | H | Br | A26 | J2 | Q23 |
| 4-134 | H | H | CH₂F | A26 | J2 | Q23 |
| 4-135 | H | H | CHF2 | A26 | J2 | Q23 |
| 4-136 | H | H | Et | A26 | J2 | Q23 |
| 4-137 | H | H | Pr | A26 | J2 | Q23 |
| 4-138 | H | H | cPr | A26 | J2 | Q23 |
| 4-139 | H | H | ethynyl | A26 | J2 | Q23 |
| 4-140 | F | H | Me | A26 | J2 | Q23 |
| 4-141 | F | F | Me | A26 | J2 | Q23 |
| 4-142 | Me | H | Me | A26 | J2 | Q23 |
| 4-143 | Me | Me | Me | A26 | J2 | Q23 |
| 4-144 | H | H | H | A27 | J2 | Q23 |
| 4-145 | H | H | Cl | A27 | J2 | Q23 |
| 4-146 | H | H | Br | A27 | J2 | Q23 |
| 4-147 | H | H | CH₂F | A27 | J2 | Q23 |
| 4-148 | H | H | CHF2 | A27 | J2 | Q23 |
| 4-149 | H | H | Et | A27 | J2 | Q23 |
| 4-150 | H | H | Pr | A27 | J2 | Q23 |
| 4-151 | H | H | cPr | A27 | J2 | Q23 |
| 4-152 | H | H | ethynyl | A27 | J2 | Q23 |
| 4-153 | F | H | Me | A27 | J2 | Q23 |
| 4-154 | F | F | Me | A27 | J2 | Q23 |
| 4-155 | Me | H | Me | A27 | J2 | Q23 |
| 4-156 | Me | Me | Me | A27 | J2 | Q23 |
| 4-157 | H | H | H | A28 | J2 | Q23 |
| 4-158 | H | H | Cl | A28 | J2 | Q23 |
| 4-159 | H | H | Br | A28 | J2 | Q23 |
| 4-160 | H | H | CH₂F | A28 | J2 | Q23 |
| 4-161 | H | H | CHF2 | A28 | J2 | Q23 |
| 4-162 | H | H | Et | A28 | J2 | Q23 |
| 4-163 | H | H | Pr | A28 | J2 | Q23 |
| 4-164 | H | H | cPr | A28 | J2 | Q23 |
| 4-165 | H | H | ethynyl | A28 | J2 | Q23 |
| 4-166 | F | H | Me | A28 | J2 | Q23 |
| 4-167 | F | F | Me | A28 | J2 | Q23 |
| 4-168 | Me | H | Me | A28 | J2 | Q23 |
| 4-169 | Me | Me | Me | A28 | J2 | Q23 |
| 4-170 | H | H | H | A29 | J2 | Q23 |
| 4-171 | H | H | Cl | A29 | J2 | Q23 |
| 4-172 | H | H | Br | A29 | J2 | Q23 |
| 4-173 | H | H | CH₂F | A29 | J2 | Q23 |
| 4-174 | H | H | CHF2 | A29 | J2 | Q23 |
| 4-175 | H | H | Et | A29 | J2 | Q23 |
| 4-176 | H | H | Pr | A29 | J2 | Q23 |
| 4-177 | H | H | cPr | A29 | J2 | Q23 |
| 4-178 | H | H | ethynyl | A29 | J2 | Q23 |
| 4-179 | F | H | Me | A29 | J2 | Q23 |
| 4-180 | F | F | Me | A29 | J2 | Q23 |
| 4-181 | Me | H | Me | A29 | J2 | Q23 |
| 4-182 | Me | Me | Me | A29 | J2 | Q23 |
| 4-183 | H | H | H | A30 | J2 | Q23 |
| 4-184 | H | H | Cl | A30 | J2 | Q23 |
| 4-185 | H | H | Br | A30 | J2 | Q23 |
| 4-186 | H | H | CH₂F | A30 | J2 | Q23 |
| 4-187 | H | H | CHF2 | A30 | J2 | Q23 |
| 4-188 | H | H | Et | A30 | J2 | Q23 |
| 4-189 | H | H | Pr | A30 | J2 | Q23 |
| 4-190 | H | H | cPr | A30 | J2 | Q23 |
| 4-191 | H | H | ethynyl | A30 | J2 | Q23 |
| 4-192 | F | H | Me | A30 | J2 | Q23 |
| 4-193 | F | F | Me | A30 | J2 | Q23 |
| 4-194 | Me | H | Me | A30 | J2 | Q23 |
| 4-195 | Me | Me | Me | A30 | J2 | Q23 |
| 4-196 | H | H | H | A31 | J2 | Q23 |
| 4-197 | H | H | Cl | A31 | J2 | Q23 |
| 4-198 | H | H | Br | A31 | J2 | Q23 |
| 4-199 | H | H | CH₂F | A31 | J2 | Q23 |
| 4-200 | H | H | CHF2 | A31 | J2 | Q23 |
| 4-201 | H | H | Et | A31 | J2 | Q23 |
| 4-202 | H | H | Pr | A31 | J2 | Q23 |
| 4-203 | H | H | cPr | A31 | J2 | Q23 |
| 4-204 | H | H | ethynyl | A31 | J2 | Q23 |
| 4-205 | F | H | Me | A31 | J2 | Q23 |
| 4-206 | F | F | Me | A31 | J2 | Q23 |
| 4-207 | Me | H | Me | A31 | J2 | Q23 |
| 4-208 | Me | Me | Me | A31 | J2 | Q23 |
| 4-209 | H | H | H | A51 | J2 | Q23 |
| 4-210 | H | H | Cl | A51 | J2 | Q23 |
| 4-211 | H | H | Br | A51 | J2 | Q23 |
| 4-212 | H | H | CH₂F | A51 | J2 | Q23 |
| 4-213 | H | H | CHF2 | A51 | J2 | Q23 |
| 4-214 | H | H | Et | A51 | J2 | Q23 |
| 4-215 | H | H | Pr | A51 | J2 | Q23 |
| 4-216 | H | H | cPr | A51 | J2 | Q23 |
| 4-217 | H | H | ethynyl | A51 | J2 | Q23 |
| 4-218 | F | H | Me | A51 | J2 | Q23 |
| 4-219 | F | F | Me | A51 | J2 | Q23 |
| 4-220 | Me | H | Me | A51 | J2 | Q23 |
| 4-221 | Me | Me | Me | A51 | J2 | Q23 |
| 4-222 | H | H | H | A1 | J1 | Q23 |
| 4-223 | H | H | Cl | A1 | J1 | Q23 |
| 4-224 | H | H | Br | A1 | J1 | Q23 |
| 4-225 | H | H | CH₂F | A1 | J1 | Q23 |
| 4-226 | H | H | CHF2 | A1 | J1 | Q23 |
| 4-227 | H | H | Et | A1 | J1 | Q23 |
| 4-228 | H | H | Pr | A1 | J1 | Q23 |
| 4-229 | H | H | cPr | A1 | J1 | Q23 |
| 4-230 | H | H | ethynyl | A1 | J1 | Q23 |
| 4-231 | F | H | Me | A1 | J1 | Q23 |
| 4-232 | F | F | Me | A1 | J1 | Q23 |
| 4-233 | Me | H | Me | A1 | J1 | Q23 |
| 4-234 | Me | Me | Me | A1 | J1 | Q23 |
| 4-235 | H | H | H | A2 | J1 | Q23 |
| 4-236 | H | H | Cl | A2 | J1 | Q23 |
| 4-237 | H | H | Br | A2 | J1 | Q23 |
| 4-238 | H | H | CH₂F | A2 | J1 | Q23 |
| 4-239 | H | H | CHF2 | A2 | J1 | Q23 |
| 4-240 | H | H | Et | A2 | J1 | Q23 |
| 4-241 | H | H | Pr | A2 | J1 | Q23 |
| 4-242 | H | H | cPr | A2 | J1 | Q23 |
| 4-243 | H | H | ethynyl | A2 | J1 | Q23 |
| 4-244 | F | H | Me | A2 | J1 | Q23 |
| 4-245 | F | F | Me | A2 | J1 | Q23 |
| 4-246 | Me | H | Me | A2 | J1 | Q23 |
| 4-247 | Me | Me | Me | A2 | J1 | Q23 |
| 4-248 | H | H | H | A6 | J1 | Q23 |
| 4-249 | H | H | Cl | A6 | J1 | Q23 |
| 4-250 | H | H | Br | A6 | J1 | Q23 |
| 4-251 | H | H | CH₂F | A6 | J1 | Q23 |
| 4-252 | H | H | CHF2 | A6 | J1 | Q23 |
| 4-253 | H | H | Et | A6 | J1 | Q23 |
| 4-254 | H | H | Pr | A6 | J1 | Q23 |
| 4-255 | H | H | cPr | A6 | J1 | Q23 |
| 4-256 | H | H | ethynyl | A6 | J1 | Q23 |
| 4-257 | F | H | Me | A6 | J1 | Q23 |
| 4-258 | F | F | Me | A6 | J1 | Q23 |
| 4-259 | Me | H | Me | A6 | J1 | Q23 |
| 4-260 | Me | Me | Me | A6 | J1 | Q23 |
| 4-261 | H | H | H | A9 | J1 | Q23 |
| 4-262 | H | H | Cl | A9 | J1 | Q23 |
| 4-263 | H | H | Br | A9 | J1 | Q23 |
| 4-264 | H | H | CH₂F | A9 | J1 | Q23 |
| 4-265 | H | H | CHF2 | A9 | J1 | Q23 |
| 4-266 | H | H | Et | A9 | J1 | Q23 |
| 4-267 | H | H | Pr | A9 | J1 | Q23 |
| 4-268 | H | H | cPr | A9 | J1 | Q23 |
| 4-269 | H | H | ethynyl | A9 | J1 | Q23 |
| 4-270 | F | H | Me | A9 | J1 | Q23 |
| 4-271 | F | F | Me | A9 | J1 | Q23 |
| 4-272 | Me | H | Me | A9 | J1 | Q23 |
| 4-273 | Me | Me | Me | A9 | J1 | Q23 |
| 4-274 | H | H | H | A11 | J1 | Q23 |
| 4-275 | H | H | Cl | A11 | J1 | Q23 |
| 4-276 | H | H | Br | A11 | J1 | Q23 |
| 4-277 | H | H | CH₂F | A11 | J1 | Q23 |
| 4-278 | H | H | CHF2 | A11 | J1 | Q23 |
| 4-279 | H | H | Et | A11 | J1 | Q23 |
| 4-280 | H | H | Pr | A11 | J1 | Q23 |
| 4-281 | H | H | cPr | A11 | J1 | Q23 |
| 4-282 | H | H | ethynyl | A11 | J1 | Q23 |
| 4-283 | F | H | Me | A11 | J1 | Q23 |
| 4-284 | F | F | Me | A11 | J1 | Q23 |
| 4-285 | Me | H | Me | A11 | J1 | Q23 |
| 4-286 | Me | Me | Me | A11 | J1 | Q23 |
| 4-287 | H | H | H | A12 | J1 | Q23 |
| 4-288 | H | H | Cl | A12 | J1 | Q23 |
| 4-289 | H | H | Br | A12 | J1 | Q23 |
| 4-290 | H | H | CH₂F | A12 | J1 | Q23 |
| 4-291 | H | H | CHF2 | A12 | J1 | Q23 |
| 4-292 | H | H | Et | A12 | J1 | Q23 |
| 4-293 | H | H | Pr | A12 | J1 | Q23 |
| 4-294 | H | H | cPr | A12 | J1 | Q23 |
| 4-295 | H | H | ethynyl | A12 | J1 | Q23 |
| 4-296 | F | H | Me | A12 | J1 | Q23 |
| 4-297 | F | F | Me | A12 | J1 | Q23 |
| 4-298 | Me | H | Me | A12 | J1 | Q23 |
| 4-299 | Me | Me | Me | A12 | J1 | Q23 |
| 4-300 | H | H | H | A19 | J1 | Q23 |
| 4-301 | H | H | Cl | A19 | J1 | Q23 |
| 4-302 | H | H | Br | A19 | J1 | Q23 |
| 4-303 | H | H | CH₂F | A19 | J1 | Q23 |
| 4-304 | H | H | CHF2 | A19 | J1 | Q23 |
| 4-305 | H | H | Et | A19 | J1 | Q23 |
| 4-306 | H | H | Pr | A19 | J1 | Q23 |
| 4-307 | H | H | cPr | A19 | J1 | Q23 |
| 4-308 | H | H | ethynyl | A19 | J1 | Q23 |
| 4-309 | F | H | Me | A19 | J1 | Q23 |
| 4-310 | F | F | Me | A19 | J1 | Q23 |
| 4-311 | Me | H | Me | A19 | J1 | Q23 |
| 4-312 | Me | Me | Me | A19 | J1 | Q23 |
| 4-313 | H | H | H | A20 | J1 | Q23 |
| 4-314 | H | H | Cl | A20 | J1 | Q23 |
| 4-315 | H | H | Br | A20 | J1 | Q23 |
| 4-316 | H | H | CH₂F | A20 | J1 | Q23 |
| 4-317 | H | H | CHF2 | A20 | J1 | Q23 |
| 4-318 | H | H | Et | A20 | J1 | Q23 |
| 4-319 | H | H | Pr | A20 | J1 | Q23 |
| 4-320 | H | H | cPr | A20 | J1 | Q23 |
| 4-321 | H | H | ethynyl | A20 | J1 | Q23 |
| 4-322 | F | H | Me | A20 | J1 | Q23 |
| 4-323 | F | F | Me | A20 | J1 | Q23 |
| 4-324 | Me | H | Me | A20 | J1 | Q23 |
| 4-325 | Me | Me | Me | A20 | J1 | Q23 |
| 4-326 | H | H | H | A21 | J1 | Q23 |
| 4-327 | H | H | Cl | A21 | J1 | Q23 |
| 4-328 | H | H | Br | A21 | J1 | Q23 |
| 4-329 | H | H | CH₂F | A21 | J1 | Q23 |
| 4-330 | H | H | CHF2 | A21 | J1 | Q23 |
| 4-331 | H | H | Et | A21 | J1 | Q23 |
| 4-332 | H | H | Pr | A21 | J1 | Q23 |
| 4-333 | H | H | cPr | A21 | J1 | Q23 |
| 4-334 | H | H | ethynyl | A21 | J1 | Q23 |
| 4-335 | F | H | Me | A21 | J1 | Q23 |
| 4-336 | F | F | Me | A21 | J1 | Q23 |
| 4-337 | Me | H | Me | A21 | J1 | Q23 |
| 4-338 | Me | Me | Me | A21 | J1 | Q23 |
| 4-339 | H | H | H | A22 | J1 | Q23 |
| 4-340 | H | H | Cl | A22 | J1 | Q23 |
| 4-341 | H | H | Br | A22 | J1 | Q23 |
| 4-342 | H | H | CH₂F | A22 | J1 | Q23 |
| 4-343 | H | H | CHF2 | A22 | J1 | Q23 |
| 4-344 | H | H | Et | A22 | J1 | Q23 |
| 4-345 | H | H | Pr | A22 | J1 | Q23 |
| 4-346 | H | H | cPr | A22 | J1 | Q23 |
| 4-347 | H | H | ethynyl | A22 | J1 | Q23 |
| 4-348 | F | H | Me | A22 | J1 | Q23 |
| 4-349 | F | F | Me | A22 | J1 | Q23 |
| 4-350 | Me | H | Me | A22 | J1 | Q23 |
| 4-351 | Me | Me | Me | A22 | J1 | Q23 |
| 4-352 | H | H | H | A26 | J1 | Q23 |
| 4-353 | H | H | Cl | A26 | J1 | Q23 |
| 4-354 | H | H | Br | A26 | J1 | Q23 |
| 4-355 | H | H | CH₂F | A26 | J1 | Q23 |
| 4-356 | H | H | CHF2 | A26 | J1 | Q23 |
| 4-357 | H | H | Et | A26 | J1 | Q23 |
| 4-358 | H | H | Pr | A26 | J1 | Q23 |
| 4-359 | H | H | cPr | A26 | J1 | Q23 |
| 4-360 | H | H | ethynyl | A26 | J1 | Q23 |
| 4-361 | F | H | Me | A26 | J1 | Q23 |
| 4-362 | F | F | Me | A26 | J1 | Q23 |
| 4-363 | Me | H | Me | A26 | J1 | Q23 |
| 4-364 | Me | Me | Me | A26 | J1 | Q23 |
| 4-365 | H | H | H | A27 | J1 | Q23 |
| 4-366 | H | H | Cl | A27 | J1 | Q23 |
| 4-367 | H | H | Br | A27 | J1 | Q23 |
| 4-368 | H | H | CH₂F | A27 | J1 | Q23 |
| 4-369 | H | H | CHF2 | A27 | J1 | Q23 |
| 4-370 | H | H | Et | A27 | J1 | Q23 |
| 4-371 | H | H | Pr | A27 | J1 | Q23 |
| 4-372 | H | H | cPr | A27 | J1 | Q23 |
| 4-373 | H | H | ethynyl | A27 | J1 | Q23 |
| 4-374 | F | H | Me | A27 | J1 | Q23 |
| 4-375 | F | F | Me | A27 | J1 | Q23 |
| 4-376 | Me | H | Me | A27 | J1 | Q23 |
| 4-377 | Me | Me | Me | A27 | J1 | Q23 |
| 4-378 | H | H | H | A28 | J1 | Q23 |
| 4-379 | H | H | Cl | A28 | J1 | Q23 |
| 4-380 | H | H | Br | A28 | J1 | Q23 |
| 4-381 | H | H | CH₂F | A28 | J1 | Q23 |
| 4-382 | H | H | CHF2 | A28 | J1 | Q23 |
| 4-383 | H | H | Et | A28 | J1 | Q23 |
| 4-384 | H | H | Pr | A28 | J1 | Q23 |
| 4-385 | H | H | cPr | A28 | J1 | Q23 |
| 4-386 | H | H | ethynyl | A28 | J1 | Q23 |
| 4-387 | F | H | Me | A28 | J1 | Q23 |
| 4-388 | F | F | Me | A28 | J1 | Q23 |
| 4-389 | Me | H | Me | A28 | J1 | Q23 |
| 4-390 | Me | Me | Me | A28 | J1 | Q23 |
| 4-391 | H | H | H | A29 | J1 | Q23 |
| 4-392 | H | H | Cl | A29 | J1 | Q23 |
| 4-393 | H | H | Br | A29 | J1 | Q23 |
| 4-394 | H | H | CH₂F | A29 | J1 | Q23 |
| 4-395 | H | H | CHF2 | A29 | J1 | Q23 |
| 4-396 | H | H | Et | A29 | J1 | Q23 |
| 4-397 | H | H | Pr | A29 | J1 | Q23 |
| 4-398 | H | H | cPr | A29 | J1 | Q23 |
| 4-399 | H | H | ethynyl | A29 | J1 | Q23 |
| 4-400 | F | H | Me | A29 | J1 | Q23 |
| 4-401 | F | F | Me | A29 | J1 | Q23 |
| 4-402 | Me | H | Me | A29 | J1 | Q23 |
| 4-403 | Me | Me | Me | A29 | J1 | Q23 |
| 4-404 | H | H | H | A30 | J1 | Q23 |
| 4-405 | H | H | Cl | A30 | J1 | Q23 |
| 4-406 | H | H | Br | A30 | J1 | Q23 |
| 4-407 | H | H | CH₂F | A30 | J1 | Q23 |
| 4-408 | H | H | CHF2 | A30 | J1 | Q23 |
| 4-409 | H | H | Et | A30 | J1 | Q23 |
| 4-410 | H | H | Pr | A30 | J1 | Q23 |
| 4-411 | H | H | cPr | A30 | J1 | Q23 |
| 4-412 | H | H | ethynyl | A30 | J1 | Q23 |
| 4-413 | F | H | Me | A30 | J1 | Q23 |
| 4-414 | F | F | Me | A30 | J1 | Q23 |
| 4-415 | Me | H | Me | A30 | J1 | Q23 |
| 4-416 | Me | Me | Me | A30 | J1 | Q23 |
| 4-417 | H | H | H | A31 | J1 | Q23 |
| 4-418 | H | H | Cl | A31 | J1 | Q23 |
| 4-419 | H | H | Br | A31 | J1 | Q23 |
| 4-420 | H | H | CH₂F | A31 | J1 | Q23 |
| 4-421 | H | H | CHF2 | A31 | J1 | Q23 |
| 4-422 | H | H | Et | A31 | J1 | Q23 |
| 4-423 | H | H | Pr | A31 | J1 | Q23 |
| 4-424 | H | H | cPr | A31 | J1 | Q23 |
| 4-425 | H | H | ethynyl | A31 | J1 | Q23 |
| 4-426 | F | H | Me | A31 | J1 | Q23 |
| 4-427 | F | F | Me | A31 | J1 | Q23 |
| 4-428 | Me | H | Me | A31 | J1 | Q23 |
| 4-429 | Me | Me | Me | A31 | J1 | Q23 |
| 4-430 | H | H | H | A51 | J1 | Q23 |
| 4-431 | H | H | Cl | A51 | J1 | Q23 |
| 4-432 | H | H | Br | A51 | J1 | Q23 |
| 4-433 | H | H | CH₂F | A51 | J1 | Q23 |
| 4-434 | H | H | CHF2 | A51 | J1 | Q23 |
| 4-435 | H | H | Et | A51 | J1 | Q23 |
| 4-436 | H | H | Pr | A51 | J1 | Q23 |
| 4-437 | H | H | cPr | A51 | J1 | Q23 |
| 4-438 | H | H | ethynyl | A51 | J1 | Q23 |
| 4-439 | F | H | Me | A51 | J1 | Q23 |
| 4-440 | F | F | Me | A51 | J1 | Q23 |
| 4-441 | Me | H | Me | A51 | J1 | Q23 |
| 4-442 | Me | Me | Me | A51 | J1 | Q23 |
| 4-443 | H | H | H | A1 | J2 | Q56 |
| 4-444 | H | H | Cl | A1 | J2 | Q56 |
| 4-445 | H | H | Br | A1 | J2 | Q56 |
| 4-446 | H | H | CH₂F | A1 | J2 | Q56 |
| 4-447 | H | H | CHF2 | A1 | J2 | Q56 |
| 4-448 | H | H | Et | A1 | J2 | Q56 |
| 4-449 | H | H | Pr | A1 | J2 | Q56 |
| 4-450 | H | H | cPr | A1 | J2 | Q56 |
| 4-451 | H | H | ethynyl | A1 | J2 | Q56 |
| 4-452 | F | H | Me | A1 | J2 | Q56 |
| 4-453 | F | F | Me | A1 | J2 | Q56 |
| 4-454 | Me | H | Me | A1 | J2 | Q56 |
| 4-455 | Me | Me | Me | A1 | J2 | Q56 |
| 4-456 | H | H | H | A2 | J2 | Q56 |
| 4-457 | H | H | Cl | A2 | J2 | Q56 |
| 4-458 | H | H | Br | A2 | J2 | Q56 |
| 4-459 | H | H | CH₂F | A2 | J2 | Q56 |
| 4-460 | H | H | CHF2 | A2 | J2 | Q56 |
| 4-461 | H | H | Et | A2 | J2 | Q56 |
| 4-462 | H | H | Pr | A2 | J2 | Q56 |
| 4-463 | H | H | cPr | A2 | J2 | Q56 |
| 4-464 | H | H | ethynyl | A2 | J2 | Q56 |
| 4-465 | F | H | Me | A2 | J2 | Q56 |
| 4-466 | F | F | Me | A2 | J2 | Q56 |
| 4-467 | Me | H | Me | A2 | J2 | Q56 |
| 4-468 | Me | Me | Me | A2 | J2 | Q56 |
| 4-469 | H | H | H | A6 | J2 | Q56 |
| 4-470 | H | H | Cl | A6 | J2 | Q56 |
| 4-471 | H | H | Br | A6 | J2 | Q56 |
| 4-472 | H | H | CH₂F | A6 | J2 | Q56 |
| 4-473 | H | H | CHF2 | A6 | J2 | Q56 |
| 4-474 | H | H | Et | A6 | J2 | Q56 |
| 4-475 | H | H | Pr | A6 | J2 | Q56 |
| 4-476 | H | H | cPr | A6 | J2 | Q56 |
| 4-477 | H | H | ethynyl | A6 | J2 | Q56 |
| 4-478 | F | H | Me | A6 | J2 | Q56 |
| 4-479 | F | F | Me | A6 | J2 | Q56 |
| 4-480 | Me | H | Me | A6 | J2 | Q56 |
| 4-481 | Me | Me | Me | A6 | J2 | Q56 |
| 4-482 | H | H | H | A9 | J2 | Q56 |
| 4-483 | H | H | Cl | A9 | J2 | Q56 |
| 4-484 | H | H | Br | A9 | J2 | Q56 |
| 4-485 | H | H | CH₂F | A9 | J2 | Q56 |
| 4-486 | H | H | CHF2 | A9 | J2 | Q56 |
| 4-487 | H | H | Et | A9 | J2 | Q56 |
| 4-488 | H | H | Pr | A9 | J2 | Q56 |
| 4-489 | H | H | cPr | A9 | J2 | Q56 |
| 4-490 | H | H | ethynyl | A9 | J2 | Q56 |
| 4-491 | F | H | Me | A9 | J2 | Q56 |
| 4-492 | F | F | Me | A9 | J2 | Q56 |
| 4-493 | Me | H | Me | A9 | J2 | Q56 |
| 4-494 | Me | Me | Me | A9 | J2 | Q56 |
| 4-495 | H | H | H | A11 | J2 | Q56 |
| 4-496 | H | H | Cl | A11 | J2 | Q56 |
| 4-497 | H | H | Br | A11 | J2 | Q56 |
| 4-498 | H | H | CH₂F | A11 | J2 | Q56 |
| 4-499 | H | H | CHF2 | A11 | J2 | Q56 |
| 4-500 | H | H | Et | A11 | J2 | Q56 |
| 4-501 | H | H | Pr | A11 | J2 | Q56 |
| 4-502 | H | H | cPr | A11 | J2 | Q56 |
| 4-503 | H | H | ethynyl | A11 | J2 | Q56 |
| 4-504 | F | H | Me | A11 | J2 | Q56 |
| 4-505 | F | F | Me | A11 | J2 | Q56 |
| 4-506 | Me | H | Me | A11 | J2 | Q56 |
| 4-507 | Me | Me | Me | A11 | J2 | Q56 |
| 4-508 | H | H | H | A12 | J2 | Q56 |
| 4-509 | H | H | Cl | A12 | J2 | Q56 |
| 4-510 | H | H | Br | A12 | J2 | Q56 |
| 4-511 | H | H | CH₂F | A12 | J2 | Q56 |
| 4-512 | H | H | CHF2 | A12 | J2 | Q56 |
| 4-513 | H | H | Et | A12 | J2 | Q56 |
| 4-514 | H | H | Pr | A12 | J2 | Q56 |
| 4-515 | H | H | cPr | A12 | J2 | Q56 |
| 4-516 | H | H | ethynyl | A12 | J2 | Q56 |
| 4-517 | F | H | Me | A12 | J2 | Q56 |
| 4-518 | F | F | Me | A12 | J2 | Q56 |
| 4-519 | Me | H | Me | A12 | J2 | Q56 |
| 4-520 | Me | Me | Me | A12 | J2 | Q56 |
| 4-521 | H | H | H | A19 | J2 | Q56 |
| 4-522 | H | H | Cl | A19 | J2 | Q56 |
| 4-523 | H | H | Br | A19 | J2 | Q56 |
| 4-524 | H | H | CH₂F | A19 | J2 | Q56 |
| 4-525 | H | H | CHF2 | A19 | J2 | Q56 |
| 4-526 | H | H | Et | A19 | J2 | Q56 |
| 4-527 | H | H | Pr | A19 | J2 | Q56 |
| 4-528 | H | H | cPr | A19 | J2 | Q56 |
| 4-529 | H | H | ethynyl | A19 | J2 | Q56 |
| 4-530 | F | H | Me | A19 | J2 | Q56 |
| 4-531 | F | F | Me | A19 | J2 | Q56 |
| 4-532 | Me | H | Me | A19 | J2 | Q56 |
| 4-533 | Me | Me | Me | A19 | J2 | Q56 |
| 4-534 | H | H | H | A20 | J2 | Q56 |
| 4-535 | H | H | Cl | A20 | J2 | Q56 |
| 4-536 | H | H | Br | A20 | J2 | Q56 |
| 4-537 | H | H | CH₂F | A20 | J2 | Q56 |
| 4-538 | H | H | CHF2 | A20 | J2 | Q56 |
| 4-539 | H | H | Et | A20 | J2 | Q56 |
| 4-540 | H | H | Pr | A20 | J2 | Q56 |
| 4-541 | H | H | cPr | A20 | J2 | Q56 |
| 4-542 | H | H | ethynyl | A20 | J2 | Q56 |
| 4-543 | F | H | Me | A20 | J2 | Q56 |
| 4-544 | F | F | Me | A20 | J2 | Q56 |
| 4-545 | Me | H | Me | A20 | J2 | Q56 |
| 4-546 | Me | Me | Me | A20 | J2 | Q56 |
| 4-547 | H | H | H | A21 | J2 | Q56 |
| 4-548 | H | H | Cl | A21 | J2 | Q56 |
| 4-549 | H | H | Br | A21 | J2 | Q56 |
| 4-550 | H | H | CH₂F | A21 | J2 | Q56 |
| 4-551 | H | H | CHF2 | A21 | J2 | Q56 |
| 4-552 | H | H | Et | A21 | J2 | Q56 |
| 4-553 | H | H | Pr | A21 | J2 | Q56 |
| 4-554 | H | H | cPr | A21 | J2 | Q56 |
| 4-555 | H | H | ethynyl | A21 | J2 | Q56 |
| 4-556 | F | H | Me | A21 | J2 | Q56 |
| 4-557 | F | F | Me | A21 | J2 | Q56 |
| 4-558 | Me | H | Me | A21 | J2 | Q56 |
| 4-559 | Me | Me | Me | A21 | J2 | Q56 |
| 4-560 | H | H | H | A22 | J2 | Q56 |
| 4-561 | H | H | Cl | A22 | J2 | Q56 |
| 4-562 | H | H | Br | A22 | J2 | Q56 |
| 4-563 | H | H | CH₂F | A22 | J2 | Q56 |
| 4-564 | H | H | CHF2 | A22 | J2 | Q56 |
| 4-565 | H | H | Et | A22 | J2 | Q56 |
| 4-566 | H | H | Pr | A22 | J2 | Q56 |
| 4-567 | H | H | cPr | A22 | J2 | Q56 |
| 4-568 | H | H | ethynyl | A22 | J2 | Q56 |
| 4-569 | F | H | Me | A22 | J2 | Q56 |
| 4-570 | F | F | Me | A22 | J2 | Q56 |
| 4-571 | Me | H | Me | A22 | J2 | Q56 |
| 4-572 | Me | Me | Me | A22 | J2 | Q56 |
| 4-573 | H | H | H | A26 | J2 | Q56 |
| 4-574 | H | H | Cl | A26 | J2 | Q56 |
| 4-575 | H | H | Br | A26 | J2 | Q56 |
| 4-576 | H | H | CH₂F | A26 | J2 | Q56 |
| 4-577 | H | H | CHF2 | A26 | J2 | Q56 |
| 4-578 | H | H | Et | A26 | J2 | Q56 |
| 4-579 | H | H | Pr | A26 | J2 | Q56 |
| 4-580 | H | H | cPr | A26 | J2 | Q56 |
| 4-581 | H | H | ethynyl | A26 | J2 | Q56 |
| 4-582 | F | H | Me | A26 | J2 | Q56 |
| 4-583 | F | F | Me | A26 | J2 | Q56 |
| 4-584 | Me | H | Me | A26 | J2 | Q56 |
| 4-585 | Me | Me | Me | A26 | J2 | Q56 |
| 4-586 | H | H | H | A27 | J2 | Q56 |
| 4-587 | H | H | Cl | A27 | J2 | Q56 |
| 4-588 | H | H | Br | A27 | J2 | Q56 |
| 4-589 | H | H | CH₂F | A27 | J2 | Q56 |
| 4-590 | H | H | CHF2 | A27 | J2 | Q56 |
| 4-591 | H | H | Et | A27 | J2 | Q56 |
| 4-592 | H | H | Pr | A27 | J2 | Q56 |
| 4-593 | H | H | cPr | A27 | J2 | Q56 |
| 4-594 | H | H | ethynyl | A27 | J2 | Q56 |
| 4-595 | F | H | Me | A27 | J2 | Q56 |
| 4-596 | F | F | Me | A27 | J2 | Q56 |
| 4-597 | Me | H | Me | A27 | J2 | Q56 |
| 4-598 | Me | Me | Me | A27 | J2 | Q56 |
| 4-599 | H | H | H | A28 | J2 | Q56 |
| 4-600 | H | H | Cl | A28 | J2 | Q56 |
| 4-601 | H | H | Br | A28 | J2 | Q56 |
| 4-602 | H | H | CH₂F | A28 | J2 | Q56 |
| 4-603 | H | H | CHF2 | A28 | J2 | Q56 |
| 4-604 | H | H | Et | A28 | J2 | Q56 |
| 4-605 | H | H | Pr | A28 | J2 | Q56 |
| 4-606 | H | H | cPr | A28 | J2 | Q56 |
| 4-607 | H | H | ethynyl | A28 | J2 | Q56 |
| 4-608 | F | H | Me | A28 | J2 | Q56 |
| 4-609 | F | F | Me | A28 | J2 | Q56 |
| 4-610 | Me | H | Me | A28 | J2 | Q56 |
| 4-611 | Me | Me | Me | A28 | J2 | Q56 |
| 4-612 | H | H | H | A29 | J2 | Q56 |
| 4-613 | H | H | Cl | A29 | J2 | Q56 |
| 4-614 | H | H | Br | A29 | J2 | Q56 |
| 4-615 | H | H | CH₂F | A29 | J2 | Q56 |
| 4-616 | H | H | CHF2 | A29 | J2 | Q56 |
| 4-617 | H | H | Et | A29 | J2 | Q56 |
| 4-618 | H | H | Pr | A29 | J2 | Q56 |
| 4-619 | H | H | cPr | A29 | J2 | Q56 |
| 4-620 | H | H | ethynyl | A29 | J2 | Q56 |
| 4-621 | F | H | Me | A29 | J2 | Q56 |
| 4-622 | F | F | Me | A29 | J2 | Q56 |
| 4-623 | Me | H | Me | A29 | J2 | Q56 |
| 4-624 | Me | Me | Me | A29 | J2 | Q56 |
| 4-625 | H | H | H | A30 | J2 | Q56 |
| 4-626 | H | H | Cl | A30 | J2 | Q56 |
| 4-627 | H | H | Br | A30 | J2 | Q56 |
| 4-628 | H | H | CH₂F | A30 | J2 | Q56 |
| 4-629 | H | H | CHF2 | A30 | J2 | Q56 |
| 4-630 | H | H | Et | A30 | J2 | Q56 |
| 4-631 | H | H | Pr | A30 | J2 | Q56 |
| 4-632 | H | H | cPr | A30 | J2 | Q56 |
| 4-633 | H | H | ethynyl | A30 | J2 | Q56 |
| 4-634 | F | H | Me | A30 | J2 | Q56 |
| 4-635 | F | F | Me | A30 | J2 | Q56 |
| 4-636 | Me | H | Me | A30 | J2 | Q56 |
| 4-637 | Me | Me | Me | A30 | J2 | Q56 |
| 4-638 | H | H | H | A31 | J2 | Q56 |
| 4-639 | H | H | Cl | A31 | J2 | Q56 |
| 4-640 | H | H | Br | A31 | J2 | Q56 |
| 4-641 | H | H | CH₂F | A31 | J2 | Q56 |
| 4-642 | H | H | CHF2 | A31 | J2 | Q56 |
| 4-643 | H | H | Et | A31 | J2 | Q56 |
| 4-644 | H | H | Pr | A31 | J2 | Q56 |
| 4-645 | H | H | cPr | A31 | J2 | Q56 |
| 4-646 | H | H | ethynyl | A31 | J2 | Q56 |
| 4-647 | F | H | Me | A31 | J2 | Q56 |
| 4-648 | F | F | Me | A31 | J2 | Q56 |
| 4-649 | Me | H | Me | A31 | J2 | Q56 |
| 4-650 | Me | Me | Me | A31 | J2 | Q56 |
| 4-651 | H | H | H | A51 | J2 | Q56 |
| 4-652 | H | H | Cl | A51 | J2 | Q56 |
| 4-653 | H | H | Br | A51 | J2 | Q56 |
| 4-654 | H | H | CH₂F | A51 | J2 | Q56 |
| 4-655 | H | H | CHF2 | A51 | J2 | Q56 |
| 4-656 | H | H | Et | A51 | J2 | Q56 |
| 4-657 | H | H | Pr | A51 | J2 | Q56 |
| 4-658 | H | H | cPr | A51 | J2 | Q56 |
| 4-659 | H | H | ethynyl | A51 | J2 | Q56 |
| 4-660 | F | H | Me | A51 | J2 | Q56 |
| 4-661 | F | F | Me | A51 | J2 | Q56 |
| 4-662 | Me | H | Me | A51 | J2 | Q56 |
| 4-663 | Me | Me | Me | A51 | J2 | Q56 |
| 4-664 | H | H | H | A1 | J1 | Q56 |
| 4-665 | H | H | Cl | A1 | J1 | Q56 |
| 4-666 | H | H | Br | A1 | J1 | Q56 |
| 4-667 | H | H | CH₂F | A1 | J1 | Q56 |
| 4-668 | H | H | CHF2 | A1 | J1 | Q56 |
| 4-669 | H | H | Et | A1 | J1 | Q56 |
| 4-670 | H | H | Pr | A1 | J1 | Q56 |
| 4-671 | H | H | cPr | A1 | J1 | Q56 |
| 4-672 | H | H | ethynyl | A1 | J1 | Q56 |
| 4-673 | F | H | Me | A1 | J1 | Q56 |
| 4-674 | F | F | Me | A1 | J1 | Q56 |
| 4-675 | Me | H | Me | A1 | J1 | Q56 |
| 4-676 | Me | Me | Me | A1 | J1 | Q56 |
| 4-677 | H | H | H | A2 | J1 | Q56 |
| 4-678 | H | H | Cl | A2 | J1 | Q56 |
| 4-679 | H | H | Br | A2 | J1 | Q56 |
| 4-680 | H | H | CH₂F | A2 | J1 | Q56 |
| 4-681 | H | H | CHF2 | A2 | J1 | Q56 |
| 4-682 | H | H | Et | A2 | J1 | Q56 |
| 4-683 | H | H | Pr | A2 | J1 | Q56 |
| 4-684 | H | H | cPr | A2 | J1 | Q56 |
| 4-685 | H | H | ethynyl | A2 | J1 | Q56 |
| 4-686 | F | H | Me | A2 | J1 | Q56 |
| 4-687 | F | F | Me | A2 | J1 | Q56 |
| 4-688 | Me | H | Me | A2 | J1 | Q56 |
| 4-689 | Me | Me | Me | A2 | J1 | Q56 |
| 4-690 | H | H | H | A6 | J1 | Q56 |
| 4-691 | H | H | Cl | A6 | J1 | Q56 |
| 4-692 | H | H | Br | A6 | J1 | Q56 |
| 4-693 | H | H | CH₂F | A6 | J1 | Q56 |
| 4-694 | H | H | CHF2 | A6 | J1 | Q56 |
| 4-695 | H | H | Et | A6 | J1 | Q56 |
| 4-696 | H | H | Pr | A6 | J1 | Q56 |
| 4-697 | H | H | cPr | A6 | J1 | Q56 |
| 4-698 | H | H | ethynyl | A6 | J1 | Q56 |
| 4-699 | F | H | Me | A6 | J1 | Q56 |
| 4-700 | F | F | Me | A6 | J1 | Q56 |
| 4-701 | Me | H | Me | A6 | J1 | Q56 |
| 4-702 | Me | Me | Me | A6 | J1 | Q56 |
| 4-703 | H | H | H | A9 | J1 | Q56 |
| 4-704 | H | H | Cl | A9 | J1 | Q56 |
| 4-705 | H | H | Br | A9 | J1 | Q56 |
| 4-706 | H | H | CH₂F | A9 | J1 | Q56 |
| 4-707 | H | H | CHF2 | A9 | J1 | Q56 |
| 4-708 | H | H | Et | A9 | J1 | Q56 |
| 4-709 | H | H | Pr | A9 | J1 | Q56 |
| 4-710 | H | H | cPr | A9 | J1 | Q56 |
| 4-711 | H | H | ethynyl | A9 | J1 | Q56 |
| 4-712 | F | H | Me | A9 | J1 | Q56 |
| 4-713 | F | F | Me | A9 | J1 | Q56 |
| 4-714 | Me | H | Me | A9 | J1 | Q56 |
| 4-715 | Me | Me | Me | A9 | J1 | Q56 |
| 4-716 | H | H | H | A11 | J1 | Q56 |
| 4-717 | H | H | Cl | A11 | J1 | Q56 |
| 4-718 | H | H | Br | A11 | J1 | Q56 |
| 4-719 | H | H | CH₂F | A11 | J1 | Q56 |
| 4-720 | H | H | CHF2 | A11 | J1 | Q56 |
| 4-721 | H | H | Et | A11 | J1 | Q56 |
| 4-722 | H | H | Pr | A11 | J1 | Q56 |
| 4-723 | H | H | cPr | A11 | J1 | Q56 |
| 4-724 | H | H | ethynyl | A11 | J1 | Q56 |
| 4-725 | F | H | Me | A11 | J1 | Q56 |
| 4-726 | F | F | Me | A11 | J1 | Q56 |
| 4-727 | Me | H | Me | A11 | J1 | Q56 |
| 4-728 | Me | Me | Me | A11 | J1 | Q56 |
| 4-729 | H | H | H | A12 | J1 | Q56 |
| 4-730 | H | H | Cl | A12 | J1 | Q56 |
| 4-731 | H | H | Br | A12 | J1 | Q56 |
| 4-732 | H | H | CH₂F | A12 | J1 | Q56 |
| 4-733 | H | H | CHF2 | A12 | J1 | Q56 |
| 4-734 | H | H | Et | A12 | J1 | Q56 |
| 4-735 | H | H | Pr | A12 | J1 | Q56 |
| 4-736 | H | H | cPr | A12 | J1 | Q56 |
| 4-737 | H | H | ethynyl | A12 | J1 | Q56 |
| 4-738 | F | H | Me | A12 | J1 | Q56 |
| 4-739 | F | F | Me | A12 | J1 | Q56 |
| 4-740 | Me | H | Me | A12 | J1 | Q56 |
| 4-741 | Me | Me | Me | A12 | J1 | Q56 |
| 4-742 | H | H | H | A19 | J1 | Q56 |
| 4-743 | H | H | Cl | A19 | J1 | Q56 |
| 4-744 | H | H | Br | A19 | J1 | Q56 |
| 4-745 | H | H | CH₂F | A19 | J1 | Q56 |
| 4-746 | H | H | CHF2 | A19 | J1 | Q56 |
| 4-747 | H | H | Et | A19 | J1 | Q56 |
| 4-748 | H | H | Pr | A19 | J1 | Q56 |
| 4-749 | H | H | cPr | A19 | J1 | Q56 |
| 4-750 | H | H | ethynyl | A19 | J1 | Q56 |
| 4-751 | F | H | Me | A19 | J1 | Q56 |
| 4-752 | F | F | Me | A19 | J1 | Q56 |
| 4-753 | Me | H | Me | A19 | J1 | Q56 |
| 4-754 | Me | Me | Me | A19 | J1 | Q56 |
| 4-755 | H | H | H | A20 | J1 | Q56 |
| 4-756 | H | H | Cl | A20 | J1 | Q56 |
| 4-757 | H | H | Br | A20 | J1 | Q56 |
| 4-758 | H | H | CH₂F | A20 | J1 | Q56 |
| 4-759 | H | H | CHF2 | A20 | J1 | Q56 |
| 4-760 | H | H | Et | A20 | J1 | Q56 |
| 4-761 | H | H | Pr | A20 | J1 | Q56 |
| 4-762 | H | H | cPr | A20 | J1 | Q56 |
| 4-763 | H | H | ethynyl | A20 | J1 | Q56 |
| 4-764 | F | H | Me | A20 | J1 | Q56 |
| 4-765 | F | F | Me | A20 | J1 | Q56 |
| 4-766 | Me | H | Me | A20 | J1 | Q56 |
| 4-767 | Me | Me | Me | A20 | J1 | Q56 |
| 4-768 | H | H | H | A21 | J1 | Q56 |
| 4-769 | H | H | Cl | A21 | J1 | Q56 |
| 4-770 | H | H | Br | A21 | J1 | Q56 |
| 4-771 | H | H | CH₂F | A21 | J1 | Q56 |
| 4-772 | H | H | CHF2 | A21 | J1 | Q56 |
| 4-773 | H | H | Et | A21 | J1 | Q56 |
| 4-774 | H | H | Pr | A21 | J1 | Q56 |
| 4-775 | H | H | cPr | A21 | J1 | Q56 |
| 4-776 | H | H | ethynyl | A21 | J1 | Q56 |
| 4-777 | F | H | Me | A21 | J1 | Q56 |
| 4-778 | F | F | Me | A21 | J1 | Q56 |
| 4-779 | Me | H | Me | A21 | J1 | Q56 |
| 4-780 | Me | Me | Me | A21 | J1 | Q56 |
| 4-781 | H | H | H | A22 | J1 | Q56 |
| 4-782 | H | H | Cl | A22 | J1 | Q56 |
| 4-783 | H | H | Br | A22 | J1 | Q56 |
| 4-784 | H | H | CH₂F | A22 | J1 | Q56 |
| 4-785 | H | H | CHF2 | A22 | J1 | Q56 |
| 4-786 | H | H | Et | A22 | J1 | Q56 |
| 4-787 | H | H | Pr | A22 | J1 | Q56 |
| 4-788 | H | H | cPr | A22 | J1 | Q56 |
| 4-789 | H | H | ethynyl | A22 | J1 | Q56 |
| 4-790 | F | H | Me | A22 | J1 | Q56 |
| 4-791 | F | F | Me | A22 | J1 | Q56 |
| 4-792 | Me | H | Me | A22 | J1 | Q56 |
| 4-793 | Me | Me | Me | A22 | J1 | Q56 |
| 4-794 | H | H | H | A26 | J1 | Q56 |
| 4-795 | H | H | Cl | A26 | J1 | Q56 |
| 4-796 | H | H | Br | A26 | J1 | Q56 |
| 4-797 | H | H | CH₂F | A26 | J1 | Q56 |
| 4-798 | H | H | CHF2 | A26 | J1 | Q56 |
| 4-799 | H | H | Et | A26 | J1 | Q56 |
| 4-800 | H | H | Pr | A26 | J1 | Q56 |
| 4-801 | H | H | cPr | A26 | J1 | Q56 |
| 4-802 | H | H | ethynyl | A26 | J1 | Q56 |
| 4-803 | F | H | Me | A26 | J1 | Q56 |
| 4-804 | F | F | Me | A26 | J1 | Q56 |
| 4-805 | Me | H | Me | A26 | J1 | Q56 |
| 4-806 | Me | Me | Me | A26 | J1 | Q56 |
| 4-807 | H | H | H | A27 | J1 | Q56 |
| 4-808 | H | H | Cl | A27 | J1 | Q56 |
| 4-809 | H | H | Br | A27 | J1 | Q56 |
| 4-810 | H | H | CH₂F | A27 | J1 | Q56 |
| 4-811 | H | H | CHF2 | A27 | J1 | Q56 |
| 4-812 | H | H | Et | A27 | J1 | Q56 |
| 4-813 | H | H | Pr | A27 | J1 | Q56 |
| 4-814 | H | H | cPr | A27 | J1 | Q56 |
| 4-815 | H | H | ethynyl | A27 | J1 | Q56 |
| 4-816 | F | H | Me | A27 | J1 | Q56 |
| 4-817 | F | F | Me | A27 | J1 | Q56 |
| 4-818 | Me | H | Me | A27 | J1 | Q56 |
| 4-819 | Me | Me | Me | A27 | J1 | Q56 |
| 4-820 | H | H | H | A28 | J1 | Q56 |
| 4-821 | H | H | Cl | A28 | J1 | Q56 |
| 4-822 | H | H | Br | A28 | J1 | Q56 |
| 4-823 | H | H | CH₂F | A28 | J1 | Q56 |
| 4-824 | H | H | CHF2 | A28 | J1 | Q56 |
| 4-825 | H | H | Et | A28 | J1 | Q56 |
| 4-826 | H | H | Pr | A28 | J1 | Q56 |
| 4-827 | H | H | cPr | A28 | J1 | Q56 |
| 4-828 | H | H | ethynyl | A28 | J1 | Q56 |
| 4-829 | F | H | Me | A28 | J1 | Q56 |
| 4-830 | F | F | Me | A28 | J1 | Q56 |
| 4-831 | Me | H | Me | A28 | J1 | Q56 |
| 4-832 | Me | Me | Me | A28 | J1 | Q56 |
| 4-833 | H | H | H | A29 | J1 | Q56 |
| 4-834 | H | H | Cl | A29 | J1 | Q56 |
| 4-835 | H | H | Br | A29 | J1 | Q56 |
| 4-836 | H | H | CH₂F | A29 | J1 | Q56 |
| 4-837 | H | H | CHF2 | A29 | J1 | Q56 |
| 4-838 | H | H | Et | A29 | J1 | Q56 |
| 4-839 | H | H | Pr | A29 | J1 | Q56 |
| 4-840 | H | H | cPr | A29 | J1 | Q56 |
| 4-841 | H | H | ethynyl | A29 | J1 | Q56 |
| 4-842 | F | H | Me | A29 | J1 | Q56 |
| 4-843 | F | F | Me | A29 | J1 | Q56 |
| 4-844 | Me | H | Me | A29 | J1 | Q56 |
| 4-845 | Me | Me | Me | A29 | J1 | Q56 |
| 4-846 | H | H | H | A30 | J1 | Q56 |
| 4-847 | H | H | Cl | A30 | J1 | Q56 |
| 4-848 | H | H | Br | A30 | J1 | Q56 |
| 4-849 | H | H | CH₂F | A30 | J1 | Q56 |
| 4-850 | H | H | CHF2 | A30 | J1 | Q56 |
| 4-851 | H | H | Et | A30 | J1 | Q56 |
| 4-852 | H | H | Pr | A30 | J1 | Q56 |
| 4-853 | H | H | cPr | A30 | J1 | Q56 |
| 4-854 | H | H | ethynyl | A30 | J1 | Q56 |
| 4-855 | F | H | Me | A30 | J1 | Q56 |
| 4-856 | F | F | Me | A30 | J1 | Q56 |
| 4-857 | Me | H | Me | A30 | J1 | Q56 |
| 4-858 | Me | Me | Me | A30 | J1 | Q56 |
| 4-859 | H | H | H | A31 | J1 | Q56 |
| 4-860 | H | H | Cl | A31 | J1 | Q56 |
| 4-861 | H | H | Br | A31 | J1 | Q56 |
| 4-862 | H | H | CH₂F | A31 | J1 | Q56 |
| 4-863 | H | H | CHF2 | A31 | J1 | Q56 |
| 4-864 | H | H | Et | A31 | J1 | Q56 |
| 4-865 | H | H | Pr | A31 | J1 | Q56 |
| 4-866 | H | H | cPr | A31 | J1 | Q56 |
| 4-867 | H | H | ethynyl | A31 | J1 | Q56 |
| 4-868 | F | H | Me | A31 | J1 | Q56 |
| 4-869 | F | F | Me | A31 | J1 | Q56 |
| 4-870 | Me | H | Me | A31 | J1 | Q56 |
| 4-871 | Me | Me | Me | A31 | J1 | Q56 |
| 4-872 | H | H | H | A51 | J1 | Q56 |
| 4-873 | H | H | Cl | A51 | J1 | Q56 |
| 4-874 | H | H | Br | A51 | J1 | Q56 |
| 4-875 | H | H | CH₂F | A51 | J1 | Q56 |
| 4-876 | H | H | CHF2 | A51 | J1 | Q56 |
| 4-877 | H | H | Et | A51 | J1 | Q56 |
| 4-878 | H | H | Pr | A51 | J1 | Q56 |
| 4-879 | H | H | cPr | A51 | J1 | Q56 |
| 4-880 | H | H | ethynyl | A51 | J1 | Q56 |
| 4-881 | F | H | Me | A51 | J1 | Q56 |
| 4-882 | F | F | Me | A51 | J1 | Q56 |
| 4-883 | Me | H | Me | A51 | J1 | Q56 |
| 4-884 | Me | Me | Me | A51 | J1 | Q56 |
| 4-885 | H | H | H | A1 | J2 | Q65 |
| 4-886 | H | H | Cl | A1 | J2 | Q65 |
| 4-887 | H | H | Br | A1 | J2 | Q65 |
| 4-888 | H | H | CH₂F | A1 | J2 | Q65 |
| 4-889 | H | H | CHF2 | A1 | J2 | Q65 |
| 4-890 | H | H | Et | A1 | J2 | Q65 |
| 4-891 | H | H | Pr | A1 | J2 | Q65 |
| 4-892 | H | H | cPr | A1 | J2 | Q65 |
| 4-893 | H | H | ethynyl | A1 | J2 | Q65 |
| 4-894 | F | H | Me | A1 | J2 | Q65 |
| 4-895 | F | F | Me | A1 | J2 | Q65 |
| 4-896 | Me | H | Me | A1 | J2 | Q65 |
| 4-897 | Me | Me | Me | A1 | J2 | Q65 |
| 4-898 | H | H | H | A2 | J2 | Q65 |
| 4-899 | H | H | Cl | A2 | J2 | Q65 |
| 4-900 | H | H | Br | A2 | J2 | Q65 |
| 4-901 | H | H | CH₂F | A2 | J2 | Q65 |
| 4-902 | H | H | CHF2 | A2 | J2 | Q65 |
| 4-903 | H | H | Et | A2 | J2 | Q65 |
| 4-904 | H | H | Pr | A2 | J2 | Q65 |
| 4-905 | H | H | cPr | A2 | J2 | Q65 |
| 4-906 | H | H | ethynyl | A2 | J2 | Q65 |
| 4-907 | F | H | Me | A2 | J2 | Q65 |
| 4-908 | F | F | Me | A2 | J2 | Q65 |
| 4-909 | Me | H | Me | A2 | J2 | Q65 |
| 4-910 | Me | Me | Me | A2 | J2 | Q65 |
| 4-911 | H | H | H | A6 | J2 | Q65 |
| 4-912 | H | H | Cl | A6 | J2 | Q65 |
| 4-913 | H | H | Br | A6 | J2 | Q65 |
| 4-914 | H | H | CH₂F | A6 | J2 | Q65 |
| 4-915 | H | H | CHF2 | A6 | J2 | Q65 |
| 4-916 | H | H | Et | A6 | J2 | Q65 |
| 4-917 | H | H | Pr | A6 | J2 | Q65 |
| 4-918 | H | H | cPr | A6 | J2 | Q65 |
| 4-919 | H | H | ethynyl | A6 | J2 | Q65 |
| 4-920 | F | H | Me | A6 | J2 | Q65 |
| 4-921 | F | F | Me | A6 | J2 | Q65 |
| 4-922 | Me | H | Me | A6 | J2 | Q65 |
| 4-923 | Me | Me | Me | A6 | J2 | Q65 |
| 4-924 | H | H | H | A9 | J2 | Q65 |
| 4-925 | H | H | Cl | A9 | J2 | Q65 |
| 4-926 | H | H | Br | A9 | J2 | Q65 |
| 4-927 | H | H | CH₂F | A9 | J2 | Q65 |
| 4-928 | H | H | CHF2 | A9 | J2 | Q65 |
| 4-929 | H | H | Et | A9 | J2 | Q65 |
| 4-930 | H | H | Pr | A9 | J2 | Q65 |
| 4-931 | H | H | cPr | A9 | J2 | Q65 |
| 4-932 | H | H | ethynyl | A9 | J2 | Q65 |
| 4-933 | F | H | Me | A9 | J2 | Q65 |
| 4-934 | F | F | Me | A9 | J2 | Q65 |
| 4-935 | Me | H | Me | A9 | J2 | Q65 |
| 4-936 | Me | Me | Me | A9 | J2 | Q65 |
| 4-937 | H | H | H | A11 | J2 | Q65 |
| 4-938 | H | H | Cl | A11 | J2 | Q65 |
| 4-939 | H | H | Br | A11 | J2 | Q65 |
| 4-940 | H | H | CH₂F | A11 | J2 | Q65 |
| 4-941 | H | H | CHF2 | A11 | J2 | Q65 |
| 4-942 | H | H | Et | A11 | J2 | Q65 |
| 4-943 | H | H | Pr | A11 | J2 | Q65 |
| 4-944 | H | H | cPr | A11 | J2 | Q65 |
| 4-945 | H | H | ethynyl | A11 | J2 | Q65 |
| 4-946 | F | H | Me | A11 | J2 | Q65 |
| 4-947 | F | F | Me | A11 | J2 | Q65 |
| 4-948 | Me | H | Me | A11 | J2 | Q65 |
| 4-949 | Me | Me | Me | A11 | J2 | Q65 |
| 4-950 | H | H | H | A12 | J2 | Q65 |
| 4-951 | H | H | Cl | A12 | J2 | Q65 |
| 4-952 | H | H | Br | A12 | J2 | Q65 |
| 4-953 | H | H | CH₂F | A12 | J2 | Q65 |
| 4-954 | H | H | CHF2 | A12 | J2 | Q65 |
| 4-955 | H | H | Et | A12 | J2 | Q65 |
| 4-956 | H | H | Pr | A12 | J2 | Q65 |
| 4-957 | H | H | cPr | A12 | J2 | Q65 |
| 4-958 | H | H | ethynyl | A12 | J2 | Q65 |
| 4-959 | F | H | Me | A12 | J2 | Q65 |
| 4-960 | F | F | Me | A12 | J2 | Q65 |
| 4-961 | Me | H | Me | A12 | J2 | Q65 |
| 4-962 | Me | Me | Me | A12 | J2 | Q65 |
| 4-963 | H | H | H | A19 | J2 | Q65 |
| 4-964 | H | H | Cl | A19 | J2 | Q65 |
| 4-965 | H | H | Br | A19 | J2 | Q65 |
| 4-966 | H | H | CH₂F | A19 | J2 | Q65 |
| 4-967 | H | H | CHF2 | A19 | J2 | Q65 |
| 4-968 | H | H | Et | A19 | J2 | Q65 |
| 4-969 | H | H | Pr | A19 | J2 | Q65 |
| 4-970 | H | H | cPr | A19 | J2 | Q65 |
| 4-971 | H | H | ethynyl | A19 | J2 | Q65 |
| 4-972 | F | H | Me | A19 | J2 | Q65 |
| 4-973 | F | F | Me | A19 | J2 | Q65 |
| 4-974 | Me | H | Me | A19 | J2 | Q65 |
| 4-975 | Me | Me | Me | A19 | J2 | Q65 |
| 4-976 | H | H | H | A20 | J2 | Q65 |
| 4-977 | H | H | Cl | A20 | J2 | Q65 |
| 4-978 | H | H | Br | A20 | J2 | Q65 |
| 4-979 | H | H | CH₂F | A20 | J2 | Q65 |
| 4-980 | H | H | CHF2 | A20 | J2 | Q65 |
| 4-981 | H | H | Et | A20 | J2 | Q65 |
| 4-982 | H | H | Pr | A20 | J2 | Q65 |
| 4-983 | H | H | cPr | A20 | J2 | Q65 |
| 4-984 | H | H | ethynyl | A20 | J2 | Q65 |
| 4-985 | F | H | Me | A20 | J2 | Q65 |
| 4-986 | F | F | Me | A20 | J2 | Q65 |
| 4-987 | Me | H | Me | A20 | J2 | Q65 |
| 4-988 | Me | Me | Me | A20 | J2 | Q65 |
| 4-989 | H | H | H | A21 | J2 | Q65 |
| 4-990 | H | H | Cl | A21 | J2 | Q65 |
| 4-991 | H | H | Br | A21 | J2 | Q65 |
| 4-992 | H | H | CH₂F | A21 | J2 | Q65 |
| 4-993 | H | H | CHF2 | A21 | J2 | Q65 |
| 4-994 | H | H | Et | A21 | J2 | Q65 |
| 4-995 | H | H | Pr | A21 | J2 | Q65 |
| 4-996 | H | H | cPr | A21 | J2 | Q65 |
| 4-997 | H | H | ethynyl | A21 | J2 | Q65 |
| 4-998 | F | H | Me | A21 | J2 | Q65 |
| 4-999 | F | F | Me | A21 | J2 | Q65 |
| 4-1000 | Me | H | Me | A21 | J2 | Q65 |
| 4-1001 | Me | Me | Me | A21 | J2 | Q65 |
| 4-1002 | H | H | H | A22 | J2 | Q65 |
| 4-1003 | H | H | Cl | A22 | J2 | Q65 |
| 4-1004 | H | H | Br | A22 | J2 | Q65 |
| 4-1005 | H | H | CH₂F | A22 | J2 | Q65 |
| 4-1006 | H | H | CHF2 | A22 | J2 | Q65 |
| 4-1007 | H | H | Et | A22 | J2 | Q65 |
| 4-1008 | H | H | Pr | A22 | J2 | Q65 |
| 4-1009 | H | H | cPr | A22 | J2 | Q65 |
| 4-1010 | H | H | ethynyl | A22 | J2 | Q65 |
| 4-1011 | F | H | Me | A22 | J2 | Q65 |
| 4-1012 | F | F | Me | A22 | J2 | Q65 |
| 4-1013 | Me | H | Me | A22 | J2 | Q65 |
| 4-1014 | Me | Me | Me | A22 | J2 | Q65 |
| 4-1015 | H | H | H | A26 | J2 | Q65 |
| 4-1016 | H | H | Cl | A26 | J2 | Q65 |
| 4-1017 | H | H | Br | A26 | J2 | Q65 |
| 4-1018 | H | H | CH₂F | A26 | J2 | Q65 |
| 4-1019 | H | H | CHF2 | A26 | J2 | Q65 |
| 4-1020 | H | H | Et | A26 | J2 | Q65 |
| 4-1021 | H | H | Pr | A26 | J2 | Q65 |
| 4-1022 | H | H | cPr | A26 | J2 | Q65 |
| 4-1023 | H | H | ethynyl | A26 | J2 | Q65 |
| 4-1024 | F | H | Me | A26 | J2 | Q65 |
| 4-1025 | F | F | Me | A26 | J2 | Q65 |
| 4-1026 | Me | H | Me | A26 | J2 | Q65 |
| 4-1027 | Me | Me | Me | A26 | J2 | Q65 |
| 4-1028 | H | H | H | A27 | J2 | Q65 |
| 4-1029 | H | H | Cl | A27 | J2 | Q65 |
| 4-1030 | H | H | Br | A27 | J2 | Q65 |
| 4-1031 | H | H | CH₂F | A27 | J2 | Q65 |
| 4-1032 | H | H | CHF2 | A27 | J2 | Q65 |
| 4-1033 | H | H | Et | A27 | J2 | Q65 |
| 4-1034 | H | H | Pr | A27 | J2 | Q65 |
| 4-1035 | H | H | cPr | A27 | J2 | Q65 |
| 4-1036 | H | H | ethynyl | A27 | J2 | Q65 |
| 4-1037 | F | H | Me | A27 | J2 | Q65 |
| 4-1038 | F | F | Me | A27 | J2 | Q65 |
| 4-1039 | Me | H | Me | A27 | J2 | Q65 |
| 4-1040 | Me | Me | Me | A27 | J2 | Q65 |
| 4-1041 | H | H | H | A28 | J2 | Q65 |
| 4-1042 | H | H | Cl | A28 | J2 | Q65 |
| 4-1043 | H | H | Br | A28 | J2 | Q65 |
| 4-1044 | H | H | CH₂F | A28 | J2 | Q65 |
| 4-1045 | H | H | CHF2 | A28 | J2 | Q65 |
| 4-1046 | H | H | Et | A28 | J2 | Q65 |
| 4-1047 | H | H | Pr | A28 | J2 | Q65 |
| 4-1048 | H | H | cPr | A28 | J2 | Q65 |
| 4-1049 | H | H | ethynyl | A28 | J2 | Q65 |
| 4-1050 | F | H | Me | A28 | J2 | Q65 |
| 4-1051 | F | F | Me | A28 | J2 | Q65 |
| 4-1052 | Me | H | Me | A28 | J2 | Q65 |
| 4-1053 | Me | Me | Me | A28 | J2 | Q65 |
| 4-1054 | H | H | H | A29 | J2 | Q65 |
| 4-1055 | H | H | Cl | A29 | J2 | Q65 |
| 4-1056 | H | H | Br | A29 | J2 | Q65 |
| 4-1057 | H | H | CH₂F | A29 | J2 | Q65 |
| 4-1058 | H | H | CHF2 | A29 | J2 | Q65 |
| 4-1059 | H | H | Et | A29 | J2 | Q65 |
| 4-1060 | H | H | Pr | A29 | J2 | Q65 |
| 4-1061 | H | H | cPr | A29 | J2 | Q65 |
| 4-1062 | H | H | ethynyl | A29 | J2 | Q65 |
| 4-1063 | F | H | Me | A29 | J2 | Q65 |
| 4-1064 | F | F | Me | A29 | J2 | Q65 |
| 4-1065 | Me | H | Me | A29 | J2 | Q65 |
| 4-1066 | Me | Me | Me | A29 | J2 | Q65 |
| 4-1067 | H | H | H | A30 | J2 | Q65 |
| 4-1068 | H | H | Cl | A30 | J2 | Q65 |
| 4-1069 | H | H | Br | A30 | J2 | Q65 |
| 4-1070 | H | H | CH₂F | A30 | J2 | Q65 |
| 4-1071 | H | H | CHF2 | A30 | J2 | Q65 |
| 4-1072 | H | H | Et | A30 | J2 | Q65 |
| 4-1073 | H | H | Pr | A30 | J2 | Q65 |
| 4-1074 | H | H | cPr | A30 | J2 | Q65 |
| 4-1075 | H | H | ethynyl | A30 | J2 | Q65 |
| 4-1076 | F | H | Me | A30 | J2 | Q65 |
| 4-1077 | F | F | Me | A30 | J2 | Q65 |
| 4-1078 | Me | H | Me | A30 | J2 | Q65 |
| 4-1079 | Me | Me | Me | A30 | J2 | Q65 |
| 4-1080 | H | H | H | A31 | J2 | Q65 |
| 4-1081 | H | H | Cl | A31 | J2 | Q65 |
| 4-1082 | H | H | Br | A31 | J2 | Q65 |
| 4-1083 | H | H | CH₂F | A31 | J2 | Q65 |
| 4-1084 | H | H | CHF2 | A31 | J2 | Q65 |
| 4-1085 | H | H | Et | A31 | J2 | Q65 |
| 4-1086 | H | H | Pr | A31 | J2 | Q65 |
| 4-1087 | H | H | cPr | A31 | J2 | Q65 |
| 4-1088 | H | H | ethynyl | A31 | J2 | Q65 |
| 4-1089 | F | H | Me | A31 | J2 | Q65 |
| 4-1090 | F | F | Me | A31 | J2 | Q65 |
| 4-1091 | Me | H | Me | A31 | J2 | Q65 |
| 4-1092 | Me | Me | Me | A31 | J2 | Q65 |
| 4-1093 | H | H | H | A51 | J2 | Q65 |
| 4-1094 | H | H | Cl | A51 | J2 | Q65 |
| 4-1095 | H | H | Br | A51 | J2 | Q65 |
| 4-1096 | H | H | CH₂F | A51 | J2 | Q65 |
| 4-1097 | H | H | CHF2 | A51 | J2 | Q65 |
| 4-1098 | H | H | Et | A51 | J2 | Q65 |
| 4-1099 | H | H | Pr | A51 | J2 | Q65 |
| 4-1100 | H | H | cPr | A51 | J2 | Q65 |
| 4-1101 | H | H | ethynyl | A51 | J2 | Q65 |
| 4-1102 | F | H | Me | A51 | J2 | Q65 |
| 4-1103 | F | F | Me | A51 | J2 | Q65 |
| 4-1104 | Me | H | Me | A51 | J2 | Q65 |
| 4-1105 | Me | Me | Me | A51 | J2 | Q65 |
| 4-1106 | H | H | H | A1 | J1 | Q65 |
| 4-1107 | H | H | Cl | A1 | J1 | Q65 |
| 4-1108 | H | H | Br | A1 | J1 | Q65 |
| 4-1109 | H | H | CH₂F | A1 | J1 | Q65 |
| 4-1110 | H | H | CHF2 | A1 | J1 | Q65 |
| 4-1111 | H | H | Et | A1 | J1 | Q65 |
| 4-1112 | H | H | Pr | A1 | J1 | Q65 |
| 4-1113 | H | H | cPr | A1 | J1 | Q65 |
| 4-1114 | H | H | ethynyl | A1 | J1 | Q65 |
| 4-1115 | F | H | Me | A1 | J1 | Q65 |
| 4-1116 | F | F | Me | A1 | J1 | Q65 |
| 4-1117 | Me | H | Me | A1 | J1 | Q65 |
| 4-1118 | Me | Me | Me | A1 | J1 | Q65 |
| 4-1119 | H | H | H | A2 | J1 | Q65 |
| 4-1120 | H | H | Cl | A2 | J1 | Q65 |
| 4-1121 | H | H | Br | A2 | J1 | Q65 |
| 4-1122 | H | H | CH₂F | A2 | J1 | Q65 |
| 4-1123 | H | H | CHF2 | A2 | J1 | Q65 |
| 4-1124 | H | H | Et | A2 | J1 | Q65 |
| 4-1125 | H | H | Pr | A2 | J1 | Q65 |
| 4-1126 | H | H | cPr | A2 | J1 | Q65 |
| 4-1127 | H | H | ethynyl | A2 | J1 | Q65 |
| 4-1128 | F | H | Me | A2 | J1 | Q65 |
| 4-1129 | F | F | Me | A2 | J1 | Q65 |
| 4-1130 | Me | H | Me | A2 | J1 | Q65 |
| 4-1131 | Me | Me | Me | A2 | J1 | Q65 |
| 4-1132 | H | H | H | A6 | J1 | Q65 |
| 4-1133 | H | H | Cl | A6 | J1 | Q65 |
| 4-1134 | H | H | Br | A6 | J1 | Q65 |
| 4-1135 | H | H | CH₂F | A6 | J1 | Q65 |
| 4-1136 | H | H | CHF2 | A6 | J1 | Q65 |
| 4-1137 | H | H | Et | A6 | J1 | Q65 |
| 4-1138 | H | H | Pr | A6 | J1 | Q65 |
| 4-1139 | H | H | cPr | A6 | J1 | Q65 |
| 4-1140 | H | H | ethynyl | A6 | J1 | Q65 |
| 4-1141 | F | H | Me | A6 | J1 | Q65 |
| 4-1142 | F | F | Me | A6 | J1 | Q65 |
| 4-1143 | Me | H | Me | A6 | J1 | Q65 |
| 4-1144 | Me | Me | Me | A6 | J1 | Q65 |
| 4-1145 | H | H | H | A9 | J1 | Q65 |
| 4-1146 | H | H | Cl | A9 | J1 | Q65 |
| 4-1147 | H | H | Br | A9 | J1 | Q65 |
| 4-1148 | H | H | CH₂F | A9 | J1 | Q65 |
| 4-1149 | H | H | CHF2 | A9 | J1 | Q65 |
| 4-1150 | H | H | Et | A9 | J1 | Q65 |
| 4-1151 | H | H | Pr | A9 | J1 | Q65 |
| 4-1152 | H | H | cPr | A9 | J1 | Q65 |
| 4-1153 | H | H | ethynyl | A9 | J1 | Q65 |
| 4-1154 | F | H | Me | A9 | J1 | Q65 |
| 4-1155 | F | F | Me | A9 | J1 | Q65 |
| 4-1156 | Me | H | Me | A9 | J1 | Q65 |
| 4-1157 | Me | Me | Me | A9 | J1 | Q65 |
| 4-1158 | H | H | H | A11 | J1 | Q65 |
| 4-1159 | H | H | Cl | A11 | J1 | Q65 |
| 4-1160 | H | H | Br | A11 | J1 | Q65 |
| 4-1161 | H | H | CH₂F | A11 | J1 | Q65 |
| 4-1162 | H | H | CHF2 | A11 | J1 | Q65 |
| 4-1163 | H | H | Et | A11 | J1 | Q65 |
| 4-1164 | H | H | Pr | A11 | J1 | Q65 |
| 4-1165 | H | H | cPr | A11 | J1 | Q65 |
| 4-1166 | H | H | ethynyl | A11 | J1 | Q65 |
| 4-1167 | F | H | Me | A11 | J1 | Q65 |
| 4-1168 | F | F | Me | A11 | J1 | Q65 |
| 4-1169 | Me | H | Me | A11 | J1 | Q65 |
| 4-1170 | Me | Me | Me | A11 | J1 | Q65 |
| 4-1171 | H | H | H | A12 | J1 | Q65 |
| 4-1172 | H | H | Cl | A12 | J1 | Q65 |
| 4-1173 | H | H | Br | A12 | J1 | Q65 |
| 4-1174 | H | H | CH₂F | A12 | J1 | Q65 |
| 4-1175 | H | H | CHF2 | A12 | J1 | Q65 |
| 4-1176 | H | H | Et | A12 | J1 | Q65 |
| 4-1177 | H | H | Pr | A12 | J1 | Q65 |
| 4-1178 | H | H | cPr | A12 | J1 | Q65 |
| 4-1179 | H | H | ethynyl | A12 | J1 | Q65 |
| 4-1180 | F | H | Me | A12 | J1 | Q65 |
| 4-1181 | F | F | Me | A12 | J1 | Q65 |
| 4-1182 | Me | H | Me | A12 | J1 | Q65 |
| 4-1183 | Me | Me | Me | A12 | J1 | Q65 |
| 4-1184 | H | H | H | A19 | J1 | Q65 |
| 4-1185 | H | H | Cl | A19 | J1 | Q65 |
| 4-1186 | H | H | Br | A19 | J1 | Q65 |
| 4-1187 | H | H | CH₂F | A19 | J1 | Q65 |
| 4-1188 | H | H | CHF2 | A19 | J1 | Q65 |
| 4-1189 | H | H | Et | A19 | J1 | Q65 |
| 4-1190 | H | H | Pr | A19 | J1 | Q65 |
| 4-1191 | H | H | cPr | A19 | J1 | Q65 |
| 4-1192 | H | H | ethynyl | A19 | J1 | Q65 |
| 4-1193 | F | H | Me | A19 | J1 | Q65 |
| 4-1194 | F | F | Me | A19 | J1 | Q65 |
| 4-1195 | Me | H | Me | A19 | J1 | Q65 |
| 4-1196 | Me | Me | Me | A19 | J1 | Q65 |
| 4-1197 | H | H | H | A20 | J1 | Q65 |
| 4-1198 | H | H | Cl | A20 | J1 | Q65 |
| 4-1199 | H | H | Br | A20 | J1 | Q65 |
| 4-1200 | H | H | CH₂F | A20 | J1 | Q65 |
| 4-1201 | H | H | CHF2 | A20 | J1 | Q65 |
| 4-1202 | H | H | Et | A20 | J1 | Q65 |
| 4-1203 | H | H | Pr | A20 | J1 | Q65 |
| 4-1204 | H | H | cPr | A20 | J1 | Q65 |
| 4-1205 | H | H | ethynyl | A20 | J1 | Q65 |
| 4-1206 | F | H | Me | A20 | J1 | Q65 |
| 4-1207 | F | F | Me | A20 | J1 | Q65 |
| 4-1208 | Me | H | Me | A20 | J1 | Q65 |
| 4-1209 | Me | Me | Me | A20 | J1 | Q65 |
| 4-1210 | H | H | H | A21 | J1 | Q65 |
| 4-1211 | H | H | Cl | A21 | J1 | Q65 |
| 4-1212 | H | H | Br | A21 | J1 | Q65 |
| 4-1213 | H | H | CH₂F | A21 | J1 | Q65 |
| 4-1214 | H | H | CHF2 | A21 | J1 | Q65 |
| 4-1215 | H | H | Et | A21 | J1 | Q65 |
| 4-1216 | H | H | Pr | A21 | J1 | Q65 |
| 4-1217 | H | H | cPr | A21 | J1 | Q65 |
| 4-1218 | H | H | ethynyl | A21 | J1 | Q65 |
| 4-1219 | F | H | Me | A21 | J1 | Q65 |
| 4-1220 | F | F | Me | A21 | J1 | Q65 |
| 4-1221 | Me | H | Me | A21 | J1 | Q65 |
| 4-1222 | Me | Me | Me | A21 | J1 | Q65 |
| 4-1223 | H | H | H | A22 | J1 | Q65 |
| 4-1224 | H | H | Cl | A22 | J1 | Q65 |
| 4-1225 | H | H | Br | A22 | J1 | Q65 |
| 4-1226 | H | H | CH₂F | A22 | J1 | Q65 |
| 4-1227 | H | H | CHF2 | A22 | J1 | Q65 |
| 4-1228 | H | H | Et | A22 | J1 | Q65 |
| 4-1229 | H | H | Pr | A22 | J1 | Q65 |
| 4-1230 | H | H | cPr | A22 | J1 | Q65 |
| 4-1231 | H | H | ethynyl | A22 | J1 | Q65 |
| 4-1232 | F | H | Me | A22 | J1 | Q65 |
| 4-1233 | F | F | Me | A22 | J1 | Q65 |
| 4-1234 | Me | H | Me | A22 | J1 | Q65 |
| 4-1235 | Me | Me | Me | A22 | J1 | Q65 |
| 4-1236 | H | H | H | A26 | J1 | Q65 |
| 4-1237 | H | H | Cl | A26 | J1 | Q65 |
| 4-1238 | H | H | Br | A26 | J1 | Q65 |
| 4-1239 | H | H | CH₂F | A26 | J1 | Q65 |
| 4-1240 | H | H | CHF2 | A26 | J1 | Q65 |
| 4-1241 | H | H | Et | A26 | J1 | Q65 |
| 4-1242 | H | H | Pr | A26 | J1 | Q65 |
| 4-1243 | H | H | cPr | A26 | J1 | Q65 |
| 4-1244 | H | H | ethynyl | A26 | J1 | Q65 |
| 4-1245 | F | H | Me | A26 | J1 | Q65 |
| 4-1246 | F | F | Me | A26 | J1 | Q65 |
| 4-1247 | Me | H | Me | A26 | J1 | Q65 |
| 4-1248 | Me | Me | Me | A26 | J1 | Q65 |
| 4-1249 | H | H | H | A27 | J1 | Q65 |
| 4-1250 | H | H | Cl | A27 | J1 | Q65 |
| 4-1251 | H | H | Br | A27 | J1 | Q65 |
| 4-1252 | H | H | CH₂F | A27 | J1 | Q65 |
| 4-1253 | H | H | CHF2 | A27 | J1 | Q65 |
| 4-1254 | H | H | Et | A27 | J1 | Q65 |
| 4-1255 | H | H | Pr | A27 | J1 | Q65 |
| 4-1256 | H | H | cPr | A27 | J1 | Q65 |
| 4-1257 | H | H | ethynyl | A27 | J1 | Q65 |
| 4-1258 | F | H | Me | A27 | J1 | Q65 |
| 4-1259 | F | F | Me | A27 | J1 | Q65 |
| 4-1260 | Me | H | Me | A27 | J1 | Q65 |
| 4-1261 | Me | Me | Me | A27 | J1 | Q65 |
| 4-1262 | H | H | H | A28 | J1 | Q65 |
| 4-1263 | H | H | Cl | A28 | J1 | Q65 |
| 4-1264 | H | H | Br | A28 | J1 | Q65 |
| 4-1265 | H | H | CH₂F | A28 | J1 | Q65 |
| 4-1266 | H | H | CHF2 | A28 | J1 | Q65 |
| 4-1267 | H | H | Et | A28 | J1 | Q65 |
| 4-1268 | H | H | Pr | A28 | J1 | Q65 |
| 4-1269 | H | H | cPr | A28 | J1 | Q65 |
| 4-1270 | H | H | ethynyl | A28 | J1 | Q65 |
| 4-1271 | F | H | Me | A28 | J1 | Q65 |
| 4-1272 | F | F | Me | A28 | J1 | Q65 |
| 4-1273 | Me | H | Me | A28 | J1 | Q65 |
| 4-1274 | Me | Me | Me | A28 | J1 | Q65 |
| 4-1275 | H | H | H | A29 | J1 | Q65 |
| 4-1276 | H | H | Cl | A29 | J1 | Q65 |
| 4-1277 | H | H | Br | A29 | J1 | Q65 |
| 4-1278 | H | H | CH₂F | A29 | J1 | Q65 |
| 4-1279 | H | H | CHF2 | A29 | J1 | Q65 |
| 4-1280 | H | H | Et | A29 | J1 | Q65 |
| 4-1281 | H | H | Pr | A29 | J1 | Q65 |
| 4-1282 | H | H | cPr | A29 | J1 | Q65 |
| 4-1283 | H | H | ethynyl | A29 | J1 | Q65 |
| 4-1284 | F | H | Me | A29 | J1 | Q65 |
| 4-1285 | F | F | Me | A29 | J1 | Q65 |
| 4-1286 | Me | H | Me | A29 | J1 | Q65 |
| 4-1287 | Me | Me | Me | A29 | J1 | Q65 |
| 4-1288 | H | H | H | A30 | J1 | Q65 |
| 4-1289 | H | H | Cl | A30 | J1 | Q65 |
| 4-1290 | H | H | Br | A30 | J1 | Q65 |
| 4-1291 | H | H | CH₂F | A30 | J1 | Q65 |
| 4-1292 | H | H | CHF2 | A30 | J1 | Q65 |
| 4-1293 | H | H | Et | A30 | J1 | Q65 |
| 4-1294 | H | H | Pr | A30 | J1 | Q65 |
| 4-1295 | H | H | cPr | A30 | J1 | Q65 |
| 4-1296 | H | H | ethynyl | A30 | J1 | Q65 |
| 4-1297 | F | H | Me | A30 | J1 | Q65 |
| 4-1298 | F | F | Me | A30 | J1 | Q65 |
| 4-1299 | Me | H | Me | A30 | J1 | Q65 |
| 4-1300 | Me | Me | Me | A30 | J1 | Q65 |
| 4-1301 | H | H | H | A31 | J1 | Q65 |
| 4-1302 | H | H | Cl | A31 | J1 | Q65 |
| 4-1303 | H | H | Br | A31 | J1 | Q65 |
| 4-1304 | H | H | CH₂F | A31 | J1 | Q65 |
| 4-1305 | H | H | CHF2 | A31 | J1 | Q65 |
| 4-1306 | H | H | Et | A31 | J1 | Q65 |
| 4-1307 | H | H | Pr | A31 | J1 | Q65 |
| 4-1308 | H | H | cPr | A31 | J1 | Q65 |
| 4-1309 | H | H | ethynyl | A31 | J1 | Q65 |
| 4-1310 | F | H | Me | A31 | J1 | Q65 |
| 4-1311 | F | F | Me | A31 | J1 | Q65 |
| 4-1312 | Me | H | Me | A31 | J1 | Q65 |
| 4-1313 | Me | Me | Me | A31 | J1 | Q65 |
| 4-1314 | H | H | H | A51 | J1 | Q65 |
| 4-1315 | H | H | Cl | A51 | J1 | Q65 |
| 4-1316 | H | H | Br | A51 | J1 | Q65 |
| 4-1317 | H | H | CH₂F | A51 | J1 | Q65 |
| 4-1318 | H | H | CHF2 | A51 | J1 | Q65 |
| 4-1319 | H | H | Et | A51 | J1 | Q65 |
| 4-1320 | H | H | Pr | A51 | J1 | Q65 |
| 4-1321 | H | H | cPr | A51 | J1 | Q65 |
| 4-1322 | H | H | ethynyl | A51 | J1 | Q65 |
| 4-1323 | F | H | Me | A51 | J1 | Q65 |
| 4-1324 | F | F | Me | A51 | J1 | Q65 |
| 4-1325 | Me | H | Me | A51 | J1 | Q65 |
| 4-1326 | Me | Me | Me | A51 | J1 | Q65 |
| 4-1327 | H | H | H | A1 | J2 | Q66 |
| 4-1328 | H | H | Cl | A1 | J2 | Q66 |
| 4-1329 | H | H | Br | A1 | J2 | Q66 |
| 4-1330 | H | H | CH₂F | A1 | J2 | Q66 |
| 4-1331 | H | H | CHF2 | A1 | J2 | Q66 |
| 4-1332 | H | H | Et | A1 | J2 | Q66 |
| 4-1333 | H | H | Pr | A1 | J2 | Q66 |
| 4-1334 | H | H | cPr | A1 | J2 | Q66 |
| 4-1335 | H | H | ethynyl | A1 | J2 | Q66 |
| 4-1336 | F | H | Me | A1 | J2 | Q66 |
| 4-1337 | F | F | Me | A1 | J2 | Q66 |
| 4-1338 | Me | H | Me | A1 | J2 | Q66 |
| 4-1339 | Me | Me | Me | A1 | J2 | Q66 |
| 4-1340 | H | H | H | A2 | J2 | Q66 |
| 4-1341 | H | H | Cl | A2 | J2 | Q66 |
| 4-1342 | H | H | Br | A2 | J2 | Q66 |
| 4-1343 | H | H | CH₂F | A2 | J2 | Q66 |
| 4-1344 | H | H | CHF2 | A2 | J2 | Q66 |
| 4-1345 | H | H | Et | A2 | J2 | Q66 |
| 4-1346 | H | H | Pr | A2 | J2 | Q66 |
| 4-1347 | H | H | cPr | A2 | J2 | Q66 |
| 4-1348 | H | H | ethynyl | A2 | J2 | Q66 |
| 4-1349 | F | H | Me | A2 | J2 | Q66 |
| 4-1350 | F | F | Me | A2 | J2 | Q66 |
| 4-1351 | Me | H | Me | A2 | J2 | Q66 |
| 4-1352 | Me | Me | Me | A2 | J2 | Q66 |
| 4-1353 | H | H | H | A6 | J2 | Q66 |
| 4-1354 | H | H | Cl | A6 | J2 | Q66 |
| 4-1355 | H | H | Br | A6 | J2 | Q66 |
| 4-1356 | H | H | CH₂F | A6 | J2 | Q66 |
| 4-1357 | H | H | CHF2 | A6 | J2 | Q66 |
| 4-1358 | H | H | Et | A6 | J2 | Q66 |
| 4-1359 | H | H | Pr | A6 | J2 | Q66 |
| 4-1360 | H | H | cPr | A6 | J2 | Q66 |
| 4-1361 | H | H | ethynyl | A6 | J2 | Q66 |
| 4-1362 | F | H | Me | A6 | J2 | Q66 |
| 4-1363 | F | F | Me | A6 | J2 | Q66 |
| 4-1364 | Me | H | Me | A6 | J2 | Q66 |
| 4-1365 | Me | Me | Me | A6 | J2 | Q66 |
| 4-1366 | H | H | H | A9 | J2 | Q66 |
| 4-1367 | H | H | Cl | A9 | J2 | Q66 |
| 4-1368 | H | H | Br | A9 | J2 | Q66 |
| 4-1369 | H | H | CH₂F | A9 | J2 | Q66 |
| 4-1370 | H | H | CHF2 | A9 | J2 | Q66 |
| 4-1371 | H | H | Et | A9 | J2 | Q66 |
| 4-1372 | H | H | Pr | A9 | J2 | Q66 |
| 4-1373 | H | H | cPr | A9 | J2 | Q66 |
| 4-1374 | H | H | ethynyl | A9 | J2 | Q66 |
| 4-1375 | F | H | Me | A9 | J2 | Q66 |
| 4-1376 | F | F | Me | A9 | J2 | Q66 |
| 4-1377 | Me | H | Me | A9 | J2 | Q66 |
| 4-1378 | Me | Me | Me | A9 | J2 | Q66 |
| 4-1379 | H | H | H | A11 | J2 | Q66 |
| 4-1380 | H | H | Cl | A11 | J2 | Q66 |
| 4-1381 | H | H | Br | A11 | J2 | Q66 |
| 4-1382 | H | H | CH₂F | A11 | J2 | Q66 |
| 4-1383 | H | H | CHF2 | A11 | J2 | Q66 |
| 4-1384 | H | H | Et | A11 | J2 | Q66 |
| 4-1385 | H | H | Pr | A11 | J2 | Q66 |
| 4-1386 | H | H | cPr | A11 | J2 | Q66 |
| 4-1387 | H | H | ethynyl | A11 | J2 | Q66 |
| 4-1388 | F | H | Me | A11 | J2 | Q66 |
| 4-1389 | F | F | Me | A11 | J2 | Q66 |
| 4-1390 | Me | H | Me | A11 | J2 | Q66 |
| 4-1391 | Me | Me | Me | A11 | J2 | Q66 |
| 4-1392 | H | H | H | A12 | J2 | Q66 |
| 4-1393 | H | H | Cl | A12 | J2 | Q66 |
| 4-1394 | H | H | Br | A12 | J2 | Q66 |
| 4-1395 | H | H | CH₂F | A12 | J2 | Q66 |
| 4-1396 | H | H | CHF2 | A12 | J2 | Q66 |
| 4-1397 | H | H | Et | A12 | J2 | Q66 |
| 4-1398 | H | H | Pr | A12 | J2 | Q66 |
| 4-1399 | H | H | cPr | A12 | J2 | Q66 |
| 4-1400 | H | H | ethynyl | A12 | J2 | Q66 |
| 4-1401 | F | H | Me | A12 | J2 | Q66 |
| 4-1402 | F | F | Me | A12 | J2 | Q66 |
| 4-1403 | Me | H | Me | A12 | J2 | Q66 |
| 4-1404 | Me | Me | Me | A12 | J2 | Q66 |
| 4-1405 | H | H | H | A19 | J2 | Q66 |
| 4-1406 | H | H | Cl | A19 | J2 | Q66 |
| 4-1407 | H | H | Br | A19 | J2 | Q66 |
| 4-1408 | H | H | CH₂F | A19 | J2 | Q66 |
| 4-1409 | H | H | CHF2 | A19 | J2 | Q66 |
| 4-1410 | H | H | Et | A19 | J2 | Q66 |
| 4-1411 | H | H | Pr | A19 | J2 | Q66 |
| 4-1412 | H | H | cPr | A19 | J2 | Q66 |
| 4-1413 | H | H | ethynyl | A19 | J2 | Q66 |
| 4-1414 | F | H | Me | A19 | J2 | Q66 |
| 4-1415 | F | F | Me | A19 | J2 | Q66 |
| 4-1416 | Me | H | Me | A19 | J2 | Q66 |
| 4-1417 | Me | Me | Me | A19 | J2 | Q66 |
| 4-1418 | H | H | H | A20 | J2 | Q66 |
| 4-1419 | H | H | Cl | A20 | J2 | Q66 |
| 4-1420 | H | H | Br | A20 | J2 | Q66 |
| 4-1421 | H | H | CH₂F | A20 | J2 | Q66 |
| 4-1422 | H | H | CHF2 | A20 | J2 | Q66 |
| 4-1423 | H | H | Et | A20 | J2 | Q66 |
| 4-1424 | H | H | Pr | A20 | J2 | Q66 |
| 4-1425 | H | H | cPr | A20 | J2 | Q66 |
| 4-1426 | H | H | ethynyl | A20 | J2 | Q66 |
| 4-1427 | F | H | Me | A20 | J2 | Q66 |
| 4-1428 | F | F | Me | A20 | J2 | Q66 |
| 4-1429 | Me | H | Me | A20 | J2 | Q66 |
| 4-1430 | Me | Me | Me | A20 | J2 | Q66 |
| 4-1431 | H | H | H | A21 | J2 | Q66 |
| 4-1432 | H | H | Cl | A21 | J2 | Q66 |
| 4-1433 | H | H | Br | A21 | J2 | Q66 |
| 4-1434 | H | H | CH₂F | A21 | J2 | Q66 |
| 4-1435 | H | H | CHF2 | A21 | J2 | Q66 |
| 4-1436 | H | H | Et | A21 | J2 | Q66 |
| 4-1437 | H | H | Pr | A21 | J2 | Q66 |
| 4-1438 | H | H | cPr | A21 | J2 | Q66 |
| 4-1439 | H | H | ethynyl | A21 | J2 | Q66 |
| 4-1440 | F | H | Me | A21 | J2 | Q66 |
| 4-1441 | F | F | Me | A21 | J2 | Q66 |
| 4-1442 | Me | H | Me | A21 | J2 | Q66 |
| 4-1443 | Me | Me | Me | A21 | J2 | Q66 |
| 4-1444 | H | H | H | A22 | J2 | Q66 |
| 4-1445 | H | H | Cl | A22 | J2 | Q66 |
| 4-1446 | H | H | Br | A22 | J2 | Q66 |
| 4-1447 | H | H | CH₂F | A22 | J2 | Q66 |
| 4-1448 | H | H | CHF2 | A22 | J2 | Q66 |
| 4-1449 | H | H | Et | A22 | J2 | Q66 |
| 4-1450 | H | H | Pr | A22 | J2 | Q66 |
| 4-1451 | H | H | cPr | A22 | J2 | Q66 |
| 4-1452 | H | H | ethynyl | A22 | J2 | Q66 |
| 4-1453 | F | H | Me | A22 | J2 | Q66 |
| 4-1454 | F | F | Me | A22 | J2 | Q66 |
| 4-1455 | Me | H | Me | A22 | J2 | Q66 |
| 4-1456 | Me | Me | Me | A22 | J2 | Q66 |
| 4-1457 | H | H | H | A26 | J2 | Q66 |
| 4-1458 | H | H | Cl | A26 | J2 | Q66 |
| 4-1459 | H | H | Br | A26 | J2 | Q66 |
| 4-1460 | H | H | CH₂F | A26 | J2 | Q66 |
| 4-1461 | H | H | CHF2 | A26 | J2 | Q66 |
| 4-1462 | H | H | Et | A26 | J2 | Q66 |
| 4-1463 | H | H | Pr | A26 | J2 | Q66 |
| 4-1464 | H | H | cPr | A26 | J2 | Q66 |
| 4-1465 | H | H | ethynyl | A26 | J2 | Q66 |
| 4-1466 | F | H | Me | A26 | J2 | Q66 |
| 4-1467 | F | F | Me | A26 | J2 | Q66 |
| 4-1468 | Me | H | Me | A26 | J2 | Q66 |
| 4-1469 | Me | Me | Me | A26 | J2 | Q66 |
| 4-1470 | H | H | H | A27 | J2 | Q66 |
| 4-1471 | H | H | Cl | A27 | J2 | Q66 |
| 4-1472 | H | H | Br | A27 | J2 | Q66 |
| 4-1473 | H | H | CH₂F | A27 | J2 | Q66 |
| 4-1474 | H | H | CHF2 | A27 | J2 | Q66 |
| 4-1475 | H | H | Et | A27 | J2 | Q66 |
| 4-1476 | H | H | Pr | A27 | J2 | Q66 |
| 4-1477 | H | H | cPr | A27 | J2 | Q66 |
| 4-1478 | H | H | ethynyl | A27 | J2 | Q66 |
| 4-1479 | F | H | Me | A27 | J2 | Q66 |
| 4-1480 | F | F | Me | A27 | J2 | Q66 |
| 4-1481 | Me | H | Me | A27 | J2 | Q66 |
| 4-1482 | Me | Me | Me | A27 | J2 | Q66 |
| 4-1483 | H | H | H | A28 | J2 | Q66 |
| 4-1484 | H | H | Cl | A28 | J2 | Q66 |
| 4-1485 | H | H | Br | A28 | J2 | Q66 |
| 4-1486 | H | H | CH₂F | A28 | J2 | Q66 |
| 4-1487 | H | H | CHF2 | A28 | J2 | Q66 |
| 4-1488 | H | H | Et | A28 | J2 | Q66 |
| 4-1489 | H | H | Pr | A28 | J2 | Q66 |
| 4-1490 | H | H | cPr | A28 | J2 | Q66 |
| 4-1491 | H | H | ethynyl | A28 | J2 | Q66 |
| 4-1492 | F | H | Me | A28 | J2 | Q66 |
| 4-1493 | F | F | Me | A28 | J2 | Q66 |
| 4-1494 | Me | H | Me | A28 | J2 | Q66 |
| 4-1495 | Me | Me | Me | A28 | J2 | Q66 |
| 4-1496 | H | H | H | A29 | J2 | Q66 |
| 4-1497 | H | H | Cl | A29 | J2 | Q66 |
| 4-1498 | H | H | Br | A29 | J2 | Q66 |
| 4-1499 | H | H | CH₂F | A29 | J2 | Q66 |
| 4-1500 | H | H | CHF2 | A29 | J2 | Q66 |
| 4-1501 | H | H | Et | A29 | J2 | Q66 |
| 4-1502 | H | H | Pr | A29 | J2 | Q66 |
| 4-1503 | H | H | cPr | A29 | J2 | Q66 |
| 4-1504 | H | H | ethynyl | A29 | J2 | Q66 |
| 4-1505 | F | H | Me | A29 | J2 | Q66 |
| 4-1506 | F | F | Me | A29 | J2 | Q66 |
| 4-1507 | Me | H | Me | A29 | J2 | Q66 |
| 4-1508 | Me | Me | Me | A29 | J2 | Q66 |
| 4-1509 | H | H | H | A30 | J2 | Q66 |
| 4-1510 | H | H | Cl | A30 | J2 | Q66 |
| 4-1511 | H | H | Br | A30 | J2 | Q66 |
| 4-1512 | H | H | CH₂F | A30 | J2 | Q66 |
| 4-1513 | H | H | CHF2 | A30 | J2 | Q66 |
| 4-1514 | H | H | Et | A30 | J2 | Q66 |
| 4-1515 | H | H | Pr | A30 | J2 | Q66 |
| 4-1516 | H | H | cPr | A30 | J2 | Q66 |
| 4-1517 | H | H | ethynyl | A30 | J2 | Q66 |
| 4-1518 | F | H | Me | A30 | J2 | Q66 |
| 4-1519 | F | F | Me | A30 | J2 | Q66 |
| 4-1520 | Me | H | Me | A30 | J2 | Q66 |
| 4-1521 | Me | Me | Me | A30 | J2 | Q66 |
| 4-1522 | H | H | H | A31 | J2 | Q66 |
| 4-1523 | H | H | Cl | A31 | J2 | Q66 |
| 4-1524 | H | H | Br | A31 | J2 | Q66 |
| 4-1525 | H | H | CH₂F | A31 | J2 | Q66 |
| 4-1526 | H | H | CHF2 | A31 | J2 | Q66 |
| 4-1527 | H | H | Et | A31 | J2 | Q66 |
| 4-1528 | H | H | Pr | A31 | J2 | Q66 |
| 4-1529 | H | H | cPr | A31 | J2 | Q66 |
| 4-1530 | H | H | ethynyl | A31 | J2 | Q66 |
| 4-1531 | F | H | Me | A31 | J2 | Q66 |
| 4-1532 | F | F | Me | A31 | J2 | Q66 |
| 4-1533 | Me | H | Me | A31 | J2 | Q66 |
| 4-1534 | Me | Me | Me | A31 | J2 | Q66 |
| 4-1535 | H | H | H | A51 | J2 | Q66 |
| 4-1536 | H | H | Cl | A51 | J2 | Q66 |
| 4-1537 | H | H | Br | A51 | J2 | Q66 |
| 4-1538 | H | H | CH₂F | A51 | J2 | Q66 |
| 4-1539 | H | H | CHF2 | A51 | J2 | Q66 |
| 4-1540 | H | H | Et | A51 | J2 | Q66 |
| 4-1541 | H | H | Pr | A51 | J2 | Q66 |
| 4-1542 | H | H | cPr | A51 | J2 | Q66 |
| 4-1543 | H | H | ethynyl | A51 | J2 | Q66 |
| 4-1544 | F | H | Me | A51 | J2 | Q66 |
| 4-1545 | F | F | Me | A51 | J2 | Q66 |
| 4-1546 | Me | H | Me | A51 | J2 | Q66 |
| 4-1547 | Me | Me | Me | A51 | J2 | Q66 |
| 4-1548 | H | H | H | A1 | J1 | Q66 |
| 4-1549 | H | H | Cl | A1 | J1 | Q66 |
| 4-1550 | H | H | Br | A1 | J1 | Q66 |
| 4-1551 | H | H | CH₂F | A1 | J1 | Q66 |
| 4-1552 | H | H | CHF2 | A1 | J1 | Q66 |
| 4-1553 | H | H | Et | A1 | J1 | Q66 |
| 4-1554 | H | H | Pr | A1 | J1 | Q66 |
| 4-1555 | H | H | cPr | A1 | J1 | Q66 |
| 4-1556 | H | H | ethynyl | A1 | J1 | Q66 |
| 4-1557 | F | H | Me | A1 | J1 | Q66 |
| 4-1558 | F | F | Me | A1 | J1 | Q66 |
| 4-1559 | Me | H | Me | A1 | J1 | Q66 |
| 4-1560 | Me | Me | Me | A1 | J1 | Q66 |
| 4-1561 | H | H | H | A2 | J1 | Q66 |
| 4-1562 | H | H | Cl | A2 | J1 | Q66 |
| 4-1563 | H | H | Br | A2 | J1 | Q66 |
| 4-1564 | H | H | CH₂F | A2 | J1 | Q66 |
| 4-1565 | H | H | CHF2 | A2 | J1 | Q66 |
| 4-1566 | H | H | Et | A2 | J1 | Q66 |
| 4-1567 | H | H | Pr | A2 | J1 | Q66 |
| 4-1568 | H | H | cPr | A2 | J1 | Q66 |
| 4-1569 | H | H | ethynyl | A2 | J1 | Q66 |
| 4-1570 | F | H | Me | A2 | J1 | Q66 |
| 4-1571 | F | F | Me | A2 | J1 | Q66 |
| 4-1572 | Me | H | Me | A2 | J1 | Q66 |
| 4-1573 | Me | Me | Me | A2 | J1 | Q66 |
| 4-1574 | H | H | H | A6 | J1 | Q66 |
| 4-1575 | H | H | Cl | A6 | J1 | Q66 |
| 4-1576 | H | H | Br | A6 | J1 | Q66 |
| 4-1577 | H | H | CH₂F | A6 | J1 | Q66 |
| 4-1578 | H | H | CHF2 | A6 | J1 | Q66 |
| 4-1579 | H | H | Et | A6 | J1 | Q66 |
| 4-1580 | H | H | Pr | A6 | J1 | Q66 |
| 4-1581 | H | H | cPr | A6 | J1 | Q66 |
| 4-1582 | H | H | ethynyl | A6 | J1 | Q66 |
| 4-1583 | F | H | Me | A6 | J1 | Q66 |
| 4-1584 | F | F | Me | A6 | J1 | Q66 |
| 4-1585 | Me | H | Me | A6 | J1 | Q66 |
| 4-1586 | Me | Me | Me | A6 | J1 | Q66 |
| 4-1587 | H | H | H | A9 | J1 | Q66 |
| 4-1588 | H | H | Cl | A9 | J1 | Q66 |
| 4-1589 | H | H | Br | A9 | J1 | Q66 |
| 4-1590 | H | H | CH₂F | A9 | J1 | Q66 |
| 4-1591 | H | H | CHF2 | A9 | J1 | Q66 |
| 4-1592 | H | H | Et | A9 | J1 | Q66 |
| 4-1593 | H | H | Pr | A9 | J1 | Q66 |
| 4-1594 | H | H | cPr | A9 | J1 | Q66 |
| 4-1595 | H | H | ethynyl | A9 | J1 | Q66 |
| 4-1596 | F | H | Me | A9 | J1 | Q66 |
| 4-1597 | F | F | Me | A9 | J1 | Q66 |
| 4-1598 | Me | H | Me | A9 | J1 | Q66 |
| 4-1599 | Me | Me | Me | A9 | J1 | Q66 |
| 4-1600 | H | H | H | A11 | J1 | Q66 |
| 4-1601 | H | H | Cl | A11 | J1 | Q66 |
| 4-1602 | H | H | Br | A11 | J1 | Q66 |
| 4-1603 | H | H | CH₂F | A11 | J1 | Q66 |
| 4-1604 | H | H | CHF2 | A11 | J1 | Q66 |
| 4-1605 | H | H | Et | A11 | J1 | Q66 |
| 4-1606 | H | H | Pr | A11 | J1 | Q66 |
| 4-1607 | H | H | cPr | A11 | J1 | Q66 |
| 4-1608 | H | H | ethynyl | A11 | J1 | Q66 |
| 4-1609 | F | H | Me | A11 | J1 | Q66 |
| 4-1610 | F | F | Me | A11 | J1 | Q66 |
| 4-1611 | Me | H | Me | A11 | J1 | Q66 |
| 4-1612 | Me | Me | Me | A11 | J1 | Q66 |
| 4-1613 | H | H | H | A12 | J1 | Q66 |
| 4-1614 | H | H | Cl | A12 | J1 | Q66 |
| 4-1615 | H | H | Br | A12 | J1 | Q66 |
| 4-1616 | H | H | CH₂F | A12 | J1 | Q66 |
| 4-1617 | H | H | CHF2 | A12 | J1 | Q66 |
| 4-1618 | H | H | Et | A12 | J1 | Q66 |
| 4-1619 | H | H | Pr | A12 | J1 | Q66 |
| 4-1620 | H | H | cPr | A12 | J1 | Q66 |
| 4-1621 | H | H | ethynyl | A12 | J1 | Q66 |
| 4-1622 | F | H | Me | A12 | J1 | Q66 |
| 4-1623 | F | F | Me | A12 | J1 | Q66 |
| 4-1624 | Me | H | Me | A12 | J1 | Q66 |
| 4-1625 | Me | Me | Me | A12 | J1 | Q66 |
| 4-1626 | H | H | H | A19 | J1 | Q66 |
| 4-1627 | H | H | Cl | A19 | J1 | Q66 |
| 4-1628 | H | H | Br | A19 | J1 | Q66 |
| 4-1629 | H | H | CH₂F | A19 | J1 | Q66 |
| 4-1630 | H | H | CHF2 | A19 | J1 | Q66 |
| 4-1631 | H | H | Et | A19 | J1 | Q66 |
| 4-1632 | H | H | Pr | A19 | J1 | Q66 |
| 4-1633 | H | H | cPr | A19 | J1 | Q66 |
| 4-1634 | H | H | ethynyl | A19 | J1 | Q66 |
| 4-1635 | F | H | Me | A19 | J1 | Q66 |
| 4-1636 | F | F | Me | A19 | J1 | Q66 |
| 4-1637 | Me | H | Me | A19 | J1 | Q66 |
| 4-1638 | Me | Me | Me | A19 | J1 | Q66 |
| 4-1639 | H | H | H | A20 | J1 | Q66 |
| 4-1640 | H | H | Cl | A20 | J1 | Q66 |
| 4-1641 | H | H | Br | A20 | J1 | Q66 |
| 4-1642 | H | H | CH₂F | A20 | J1 | Q66 |
| 4-1643 | H | H | CHF2 | A20 | J1 | Q66 |
| 4-1644 | H | H | Et | A20 | J1 | Q66 |
| 4-1645 | H | H | Pr | A20 | J1 | Q66 |
| 4-1646 | H | H | cPr | A20 | J1 | Q66 |
| 4-1647 | H | H | ethynyl | A20 | J1 | Q66 |
| 4-1648 | F | H | Me | A20 | J1 | Q66 |
| 4-1649 | F | F | Me | A20 | J1 | Q66 |
| 4-1650 | Me | H | Me | A20 | J1 | Q66 |
| 4-1651 | Me | Me | Me | A20 | J1 | Q66 |
| 4-1652 | H | H | H | A21 | J1 | Q66 |
| 4-1653 | H | H | Cl | A21 | J1 | Q66 |
| 4-1654 | H | H | Br | A21 | J1 | Q66 |
| 4-1655 | H | H | CH₂F | A21 | J1 | Q66 |
| 4-1656 | H | H | CHF2 | A21 | J1 | Q66 |
| 4-1657 | H | H | Et | A21 | J1 | Q66 |
| 4-1658 | H | H | Pr | A21 | J1 | Q66 |
| 4-1659 | H | H | cPr | A21 | J1 | Q66 |
| 4-1660 | H | H | ethynyl | A21 | J1 | Q66 |
| 4-1661 | F | H | Me | A21 | J1 | Q66 |
| 4-1662 | F | F | Me | A21 | J1 | Q66 |
| 4-1663 | Me | H | Me | A21 | J1 | Q66 |
| 4-1664 | Me | Me | Me | A21 | J1 | Q66 |
| 4-1665 | H | H | H | A22 | J1 | Q66 |
| 4-1666 | H | H | Cl | A22 | J1 | Q66 |
| 4-1667 | H | H | Br | A22 | J1 | Q66 |
| 4-1668 | H | H | CH₂F | A22 | J1 | Q66 |
| 4-1669 | H | H | CHF2 | A22 | J1 | Q66 |
| 4-1670 | H | H | Et | A22 | J1 | Q66 |
| 4-1671 | H | H | Pr | A22 | J1 | Q66 |
| 4-1672 | H | H | cPr | A22 | J1 | Q66 |
| 4-1673 | H | H | ethynyl | A22 | J1 | Q66 |
| 4-1674 | F | H | Me | A22 | J1 | Q66 |
| 4-1675 | F | F | Me | A22 | J1 | Q66 |
| 4-1676 | Me | H | Me | A22 | J1 | Q66 |
| 4-1677 | Me | Me | Me | A22 | J1 | Q66 |
| 4-1678 | H | H | H | A26 | J1 | Q66 |
| 4-1679 | H | H | Cl | A26 | J1 | Q66 |
| 4-1680 | H | H | Br | A26 | J1 | Q66 |
| 4-1681 | H | H | CH₂F | A26 | J1 | Q66 |
| 4-1682 | H | H | CHF2 | A26 | J1 | Q66 |
| 4-1683 | H | H | Et | A26 | J1 | Q66 |
| 4-1684 | H | H | Pr | A26 | J1 | Q66 |
| 4-1685 | H | H | cPr | A26 | J1 | Q66 |
| 4-1686 | H | H | ethynyl | A26 | J1 | Q66 |
| 4-1687 | F | H | Me | A26 | J1 | Q66 |
| 4-1688 | F | F | Me | A26 | J1 | Q66 |
| 4-1689 | Me | H | Me | A26 | J1 | Q66 |
| 4-1690 | Me | Me | Me | A26 | J1 | Q66 |
| 4-1691 | H | H | H | A27 | J1 | Q66 |
| 4-1692 | H | H | Cl | A27 | J1 | Q66 |
| 4-1693 | H | H | Br | A27 | J1 | Q66 |
| 4-1694 | H | H | CH₂F | A27 | J1 | Q66 |
| 4-1695 | H | H | CHF2 | A27 | J1 | Q66 |
| 4-1696 | H | H | Et | A27 | J1 | Q66 |
| 4-1697 | H | H | Pr | A27 | J1 | Q66 |
| 4-1698 | H | H | cPr | A27 | J1 | Q66 |
| 4-1699 | H | H | ethynyl | A27 | J1 | Q66 |
| 4-1700 | F | H | Me | A27 | J1 | Q66 |
| 4-1701 | F | F | Me | A27 | J1 | Q66 |
| 4-1702 | Me | H | Me | A27 | J1 | Q66 |
| 4-1703 | Me | Me | Me | A27 | J1 | Q66 |
| 4-1704 | H | H | H | A28 | J1 | Q66 |
| 4-1705 | H | H | Cl | A28 | J1 | Q66 |
| 4-1706 | H | H | Br | A28 | J1 | Q66 |
| 4-1707 | H | H | CH₂F | A28 | J1 | Q66 |
| 4-1708 | H | H | CHF2 | A28 | J1 | Q66 |
| 4-1709 | H | H | Et | A28 | J1 | Q66 |
| 4-1710 | H | H | Pr | A28 | J1 | Q66 |
| 4-1711 | H | H | cPr | A28 | J1 | Q66 |
| 4-1712 | H | H | ethynyl | A28 | J1 | Q66 |
| 4-1713 | F | H | Me | A28 | J1 | Q66 |
| 4-1714 | F | F | Me | A28 | J1 | Q66 |
| 4-1715 | Me | H | Me | A28 | J1 | Q66 |
| 4-1716 | Me | Me | Me | A28 | J1 | Q66 |
| 4-1717 | H | H | H | A29 | J1 | Q66 |
| 4-1718 | H | H | Cl | A29 | J1 | Q66 |
| 4-1719 | H | H | Br | A29 | J1 | Q66 |
| 4-1720 | H | H | CH₂F | A29 | J1 | Q66 |
| 4-1721 | H | H | CHF2 | A29 | J1 | Q66 |
| 4-1722 | H | H | Et | A29 | J1 | Q66 |
| 4-1723 | H | H | Pr | A29 | J1 | Q66 |
| 4-1724 | H | H | cPr | A29 | J1 | Q66 |
| 4-1725 | H | H | ethynyl | A29 | J1 | Q66 |
| 4-1726 | F | H | Me | A29 | J1 | Q66 |
| 4-1727 | F | F | Me | A29 | J1 | Q66 |
| 4-1728 | Me | H | Me | A29 | J1 | Q66 |
| 4-1729 | Me | Me | Me | A29 | J1 | Q66 |
| 4-1730 | H | H | H | A30 | J1 | Q66 |
| 4-1731 | H | H | Cl | A30 | J1 | Q66 |
| 4-1732 | H | H | Br | A30 | J1 | Q66 |
| 4-1733 | H | H | CH₂F | A30 | J1 | Q66 |
| 4-1734 | H | H | CHF2 | A30 | J1 | Q66 |
| 4-1735 | H | H | Et | A30 | J1 | Q66 |
| 4-1736 | H | H | Pr | A30 | J1 | Q66 |
| 4-1737 | H | H | cPr | A30 | J1 | Q66 |
| 4-1738 | H | H | ethynyl | A30 | J1 | Q66 |
| 4-1739 | F | H | Me | A30 | J1 | Q66 |
| 4-1740 | F | F | Me | A30 | J1 | Q66 |
| 4-1741 | Me | H | Me | A30 | J1 | Q66 |
| 4-1742 | Me | Me | Me | A30 | J1 | Q66 |
| 4-1743 | H | H | H | A31 | J1 | Q66 |
| 4-1744 | H | H | Cl | A31 | J1 | Q66 |
| 4-1745 | H | H | Br | A31 | J1 | Q66 |
| 4-1746 | H | H | CH₂F | A31 | J1 | Q66 |
| 4-1747 | H | H | CHF2 | A31 | J1 | Q66 |
| 4-1748 | H | H | Et | A31 | J1 | Q66 |
| 4-1749 | H | H | Pr | A31 | J1 | Q66 |
| 4-1750 | H | H | cPr | A31 | J1 | Q66 |
| 4-1751 | H | H | ethynyl | A31 | J1 | Q66 |
| 4-1752 | F | H | Me | A31 | J1 | Q66 |
| 4-1753 | F | F | Me | A31 | J1 | Q66 |
| 4-1754 | Me | H | Me | A31 | J1 | Q66 |
| 4-1755 | Me | Me | Me | A31 | J1 | Q66 |
| 4-1756 | H | H | H | A51 | J1 | Q66 |
| 4-1757 | H | H | Cl | A51 | J1 | Q66 |
| 4-1758 | H | H | Br | A51 | J1 | Q66 |
| 4-1759 | H | H | CH₂F | A51 | J1 | Q66 |
| 4-1760 | H | H | CHF2 | A51 | J1 | Q66 |
| 4-1761 | H | H | Et | A51 | J1 | Q66 |
| 4-1762 | H | H | Pr | A51 | J1 | Q66 |
| 4-1763 | H | H | cPr | A51 | J1 | Q66 |
| 4-1764 | H | H | ethynyl | A51 | J1 | Q66 |
| 4-1765 | F | H | Me | A51 | J1 | Q66 |
| 4-1766 | F | F | Me | A51 | J1 | Q66 |
| 4-1767 | Me | H | Me | A51 | J1 | Q66 |
| 4-1768 | Me | Me | Me | A51 | J1 | Q66 |
| 4-1769 | H | H | H | A1 | J2 | Q98 |
| 4-1770 | H | H | Cl | A1 | J2 | Q98 |
| 4-1771 | H | H | Br | A1 | J2 | Q98 |
| 4-1772 | H | H | CH₂F | A1 | J2 | Q98 |
| 4-1773 | H | H | CHF2 | A1 | J2 | Q98 |
| 4-1774 | H | H | Et | A1 | J2 | Q98 |
| 4-1775 | H | H | Pr | A1 | J2 | Q98 |
| 4-1776 | H | H | cPr | A1 | J2 | Q98 |
| 4-1777 | H | H | ethynyl | A1 | J2 | Q98 |
| 4-1778 | F | H | Me | A1 | J2 | Q98 |
| 4-1779 | F | F | Me | A1 | J2 | Q98 |
| 4-1780 | Me | H | Me | A1 | J2 | Q98 |
| 4-1781 | Me | Me | Me | A1 | J2 | Q98 |
| 4-1782 | H | H | H | A2 | J2 | Q98 |
| 4-1783 | H | H | Cl | A2 | J2 | Q98 |
| 4-1784 | H | H | Br | A2 | J2 | Q98 |
| 4-1785 | H | H | CH₂F | A2 | J2 | Q98 |
| 4-1786 | H | H | CHF2 | A2 | J2 | Q98 |
| 4-1787 | H | H | Et | A2 | J2 | Q98 |
| 4-1788 | H | H | Pr | A2 | J2 | Q98 |
| 4-1789 | H | H | cPr | A2 | J2 | Q98 |
| 4-1790 | H | H | ethynyl | A2 | J2 | Q98 |
| 4-1791 | F | H | Me | A2 | J2 | Q98 |
| 4-1792 | F | F | Me | A2 | J2 | Q98 |
| 4-1793 | Me | H | Me | A2 | J2 | Q98 |
| 4-1794 | Me | Me | Me | A2 | J2 | Q98 |
| 4-1795 | H | H | H | A6 | J2 | Q98 |
| 4-1796 | H | H | Cl | A6 | J2 | Q98 |
| 4-1797 | H | H | Br | A6 | J2 | Q98 |
| 4-1798 | H | H | CH₂F | A6 | J2 | Q98 |
| 4-1799 | H | H | CHF2 | A6 | J2 | Q98 |
| 4-1800 | H | H | Et | A6 | J2 | Q98 |
| 4-1801 | H | H | Pr | A6 | J2 | Q98 |
| 4-1802 | H | H | cPr | A6 | J2 | Q98 |
| 4-1803 | H | H | ethynyl | A6 | J2 | Q98 |
| 4-1804 | F | H | Me | A6 | J2 | Q98 |
| 4-1805 | F | F | Me | A6 | J2 | Q98 |
| 4-1806 | Me | H | Me | A6 | J2 | Q98 |
| 4-1807 | Me | Me | Me | A6 | J2 | Q98 |
| 4-1808 | H | H | H | A9 | J2 | Q98 |
| 4-1809 | H | H | Cl | A9 | J2 | Q98 |
| 4-1810 | H | H | Br | A9 | J2 | Q98 |
| 4-1811 | H | H | CH₂F | A9 | J2 | Q98 |
| 4-1812 | H | H | CHF2 | A9 | J2 | Q98 |
| 4-1813 | H | H | Et | A9 | J2 | Q98 |
| 4-1814 | H | H | Pr | A9 | J2 | Q98 |
| 4-1815 | H | H | cPr | A9 | J2 | Q98 |
| 4-1816 | H | H | ethynyl | A9 | J2 | Q98 |
| 4-1817 | F | H | Me | A9 | J2 | Q98 |
| 4-1818 | F | F | Me | A9 | J2 | Q98 |
| 4-1819 | Me | H | Me | A9 | J2 | Q98 |
| 4-1820 | Me | Me | Me | A9 | J2 | Q98 |
| 4-1821 | H | H | H | A11 | J2 | Q98 |
| 4-1822 | H | H | Cl | A11 | J2 | Q98 |
| 4-1823 | H | H | Br | A11 | J2 | Q98 |
| 4-1824 | H | H | CH₂F | A11 | J2 | Q98 |
| 4-1825 | H | H | CHF2 | A11 | J2 | Q98 |
| 4-1826 | H | H | Et | A11 | J2 | Q98 |
| 4-1827 | H | H | Pr | A11 | J2 | Q98 |
| 4-1828 | H | H | cPr | A11 | J2 | Q98 |
| 4-1829 | H | H | ethynyl | A11 | J2 | Q98 |
| 4-1830 | F | H | Me | A11 | J2 | Q98 |
| 4-1831 | F | F | Me | A11 | J2 | Q98 |
| 4-1832 | Me | H | Me | A11 | J2 | Q98 |
| 4-1833 | Me | Me | Me | A11 | J2 | Q98 |
| 4-1834 | H | H | H | A12 | J2 | Q98 |
| 4-1835 | H | H | Cl | A12 | J2 | Q98 |
| 4-1836 | H | H | Br | A12 | J2 | Q98 |
| 4-1837 | H | H | CH₂F | A12 | J2 | Q98 |
| 4-1838 | H | H | CHF2 | A12 | J2 | Q98 |
| 4-1839 | H | H | Et | A12 | J2 | Q98 |
| 4-1840 | H | H | Pr | A12 | J2 | Q98 |
| 4-1841 | H | H | cPr | A12 | J2 | Q98 |
| 4-1842 | H | H | ethynyl | A12 | J2 | Q98 |
| 4-1843 | F | H | Me | A12 | J2 | Q98 |
| 4-1844 | F | F | Me | A12 | J2 | Q98 |
| 4-1845 | Me | H | Me | A12 | J2 | Q98 |
| 4-1846 | Me | Me | Me | A12 | J2 | Q98 |
| 4-1847 | H | H | H | A19 | J2 | Q98 |
| 4-1848 | H | H | Cl | A19 | J2 | Q98 |
| 4-1849 | H | H | Br | A19 | J2 | Q98 |
| 4-1850 | H | H | CH₂F | A19 | J2 | Q98 |
| 4-1851 | H | H | CHF2 | A19 | J2 | Q98 |
| 4-1852 | H | H | Et | A19 | J2 | Q98 |
| 4-1853 | H | H | Pr | A19 | J2 | Q98 |
| 4-1854 | H | H | cPr | A19 | J2 | Q98 |
| 4-1855 | H | H | ethynyl | A19 | J2 | Q98 |
| 4-1856 | F | H | Me | A19 | J2 | Q98 |
| 4-1857 | F | F | Me | A19 | J2 | Q98 |
| 4-1858 | Me | H | Me | A19 | J2 | Q98 |
| 4-1859 | Me | Me | Me | A19 | J2 | Q98 |
| 4-1860 | H | H | H | A20 | J2 | Q98 |
| 4-1861 | H | H | Cl | A20 | J2 | Q98 |
| 4-1862 | H | H | Br | A20 | J2 | Q98 |
| 4-1863 | H | H | CH₂F | A20 | J2 | Q98 |
| 4-1864 | H | H | CHF2 | A20 | J2 | Q98 |
| 4-1865 | H | H | Et | A20 | J2 | Q98 |
| 4-1866 | H | H | Pr | A20 | J2 | Q98 |
| 4-1867 | H | H | cPr | A20 | J2 | Q98 |
| 4-1868 | H | H | ethynyl | A20 | J2 | Q98 |
| 4-1869 | F | H | Me | A20 | J2 | Q98 |
| 4-1870 | F | F | Me | A20 | J2 | Q98 |
| 4-1871 | Me | H | Me | A20 | J2 | Q98 |
| 4-1872 | Me | Me | Me | A20 | J2 | Q98 |
| 4-1873 | H | H | H | A21 | J2 | Q98 |
| 4-1874 | H | H | Cl | A21 | J2 | Q98 |
| 4-1875 | H | H | Br | A21 | J2 | Q98 |
| 4-1876 | H | H | CH₂F | A21 | J2 | Q98 |
| 4-1877 | H | H | CHF2 | A21 | J2 | Q98 |
| 4-1878 | H | H | Et | A21 | J2 | Q98 |
| 4-1879 | H | H | Pr | A21 | J2 | Q98 |
| 4-1880 | H | H | cPr | A21 | J2 | Q98 |
| 4-1881 | H | H | ethynyl | A21 | J2 | Q98 |
| 4-1882 | F | H | Me | A21 | J2 | Q98 |
| 4-1883 | F | F | Me | A21 | J2 | Q98 |
| 4-1884 | Me | H | Me | A21 | J2 | Q98 |
| 4-1885 | Me | Me | Me | A21 | J2 | Q98 |
| 4-1886 | H | H | H | A22 | J2 | Q98 |
| 4-1887 | H | H | Cl | A22 | J2 | Q98 |
| 4-1888 | H | H | Br | A22 | J2 | Q98 |
| 4-1889 | H | H | CH₂F | A22 | J2 | Q98 |
| 4-1890 | H | H | CHF2 | A22 | J2 | Q98 |
| 4-1891 | H | H | Et | A22 | J2 | Q98 |
| 4-1892 | H | H | Pr | A22 | J2 | Q98 |
| 4-1893 | H | H | cPr | A22 | J2 | Q98 |
| 4-1894 | H | H | ethynyl | A22 | J2 | Q98 |
| 4-1895 | F | H | Me | A22 | J2 | Q98 |
| 4-1896 | F | F | Me | A22 | J2 | Q98 |
| 4-1897 | Me | H | Me | A22 | J2 | Q98 |
| 4-1898 | Me | Me | Me | A22 | J2 | Q98 |
| 4-1899 | H | H | H | A26 | J2 | Q98 |
| 4-1900 | H | H | Cl | A26 | J2 | Q98 |
| 4-1901 | H | H | Br | A26 | J2 | Q98 |
| 4-1902 | H | H | CH₂F | A26 | J2 | Q98 |
| 4-1903 | H | H | CHF2 | A26 | J2 | Q98 |
| 4-1904 | H | H | Et | A26 | J2 | Q98 |
| 4-1905 | H | H | Pr | A26 | J2 | Q98 |
| 4-1906 | H | H | cPr | A26 | J2 | Q98 |
| 4-1907 | H | H | ethynyl | A26 | J2 | Q98 |
| 4-1908 | F | H | Me | A26 | J2 | Q98 |
| 4-1909 | F | F | Me | A26 | J2 | Q98 |
| 4-1910 | Me | H | Me | A26 | J2 | Q98 |
| 4-1911 | Me | Me | Me | A26 | J2 | Q98 |
| 4-1912 | H | H | H | A27 | J2 | Q98 |
| 4-1913 | H | H | Cl | A27 | J2 | Q98 |
| 4-1914 | H | H | Br | A27 | J2 | Q98 |
| 4-1915 | H | H | CH₂F | A27 | J2 | Q98 |
| 4-1916 | H | H | CHF2 | A27 | J2 | Q98 |
| 4-1917 | H | H | Et | A27 | J2 | Q98 |
| 4-1918 | H | H | Pr | A27 | J2 | Q98 |
| 4-1919 | H | H | cPr | A27 | J2 | Q98 |
| 4-1920 | H | H | ethynyl | A27 | J2 | Q98 |
| 4-1921 | F | H | Me | A27 | J2 | Q98 |
| 4-1922 | F | F | Me | A27 | J2 | Q98 |
| 4-1923 | Me | H | Me | A27 | J2 | Q98 |
| 4-1924 | Me | Me | Me | A27 | J2 | Q98 |
| 4-1925 | H | H | H | A28 | J2 | Q98 |
| 4-1926 | H | H | Cl | A28 | J2 | Q98 |
| 4-1927 | H | H | Br | A28 | J2 | Q98 |
| 4-1928 | H | H | CH₂F | A28 | J2 | Q98 |
| 4-1929 | H | H | CHF2 | A28 | J2 | Q98 |
| 4-1930 | H | H | Et | A28 | J2 | Q98 |
| 4-1931 | H | H | Pr | A28 | J2 | Q98 |
| 4-1932 | H | H | cPr | A28 | J2 | Q98 |
| 4-1933 | H | H | ethynyl | A28 | J2 | Q98 |
| 4-1934 | F | H | Me | A28 | J2 | Q98 |
| 4-1935 | F | F | Me | A28 | J2 | Q98 |
| 4-1936 | Me | H | Me | A28 | J2 | Q98 |
| 4-1937 | Me | Me | Me | A28 | J2 | Q98 |
| 4-1938 | H | H | H | A29 | J2 | Q98 |
| 4-1939 | H | H | Cl | A29 | J2 | Q98 |
| 4-1940 | H | H | Br | A29 | J2 | Q98 |
| 4-1941 | H | H | CH₂F | A29 | J2 | Q98 |
| 4-1942 | H | H | CHF2 | A29 | J2 | Q98 |
| 4-1943 | H | H | Et | A29 | J2 | Q98 |
| 4-1944 | H | H | Pr | A29 | J2 | Q98 |
| 4-1945 | H | H | cPr | A29 | J2 | Q98 |
| 4-1946 | H | H | ethynyl | A29 | J2 | Q98 |
| 4-1947 | F | H | Me | A29 | J2 | Q98 |
| 4-1948 | F | F | Me | A29 | J2 | Q98 |
| 4-1949 | Me | H | Me | A29 | J2 | Q98 |
| 4-1950 | Me | Me | Me | A29 | J2 | Q98 |
| 4-1951 | H | H | H | A30 | J2 | Q98 |
| 4-1952 | H | H | Cl | A30 | J2 | Q98 |
| 4-1953 | H | H | Br | A30 | J2 | Q98 |
| 4-1954 | H | H | CH₂F | A30 | J2 | Q98 |
| 4-1955 | H | H | CHF2 | A30 | J2 | Q98 |
| 4-1956 | H | H | Et | A30 | J2 | Q98 |
| 4-1957 | H | H | Pr | A30 | J2 | Q98 |
| 4-1958 | H | H | cPr | A30 | J2 | Q98 |
| 4-1959 | H | H | ethynyl | A30 | J2 | Q98 |
| 4-1960 | F | H | Me | A30 | J2 | Q98 |
| 4-1961 | F | F | Me | A30 | J2 | Q98 |
| 4-1962 | Me | H | Me | A30 | J2 | Q98 |
| 4-1963 | Me | Me | Me | A30 | J2 | Q98 |
| 4-1964 | H | H | H | A31 | J2 | Q98 |
| 4-1965 | H | H | Cl | A31 | J2 | Q98 |
| 4-1966 | H | H | Br | A31 | J2 | Q98 |
| 4-1967 | H | H | CH₂F | A31 | J2 | Q98 |
| 4-1968 | H | H | CHF2 | A31 | J2 | Q98 |
| 4-1969 | H | H | Et | A31 | J2 | Q98 |
| 4-1970 | H | H | Pr | A31 | J2 | Q98 |
| 4-1971 | H | H | cPr | A31 | J2 | Q98 |
| 4-1972 | H | H | ethynyl | A31 | J2 | Q98 |
| 4-1973 | F | H | Me | A31 | J2 | Q98 |
| 4-1974 | F | F | Me | A31 | J2 | Q98 |
| 4-1975 | Me | H | Me | A31 | J2 | Q98 |
| 4-1976 | Me | Me | Me | A31 | J2 | Q98 |
| 4-1977 | H | H | H | A51 | J2 | Q98 |
| 4-1978 | H | H | Cl | A51 | J2 | Q98 |
| 4-1979 | H | H | Br | A51 | J2 | Q98 |
| 4-1980 | H | H | CH₂F | A51 | J2 | Q98 |
| 4-1981 | H | H | CHF2 | A51 | J2 | Q98 |
| 4-1982 | H | H | Et | A51 | J2 | Q98 |
| 4-1983 | H | H | Pr | A51 | J2 | Q98 |
| 4-1984 | H | H | cPr | A51 | J2 | Q98 |
| 4-1985 | H | H | ethynyl | A51 | J2 | Q98 |
| 4-1986 | F | H | Me | A51 | J2 | Q98 |
| 4-1987 | F | F | Me | A51 | J2 | Q98 |
| 4-1988 | Me | H | Me | A51 | J2 | Q98 |
| 4-1989 | Me | Me | Me | A51 | J2 | Q98 |
| 4-1990 | H | H | H | A1 | J1 | Q98 |
| 4-1991 | H | H | Cl | A1 | J1 | Q98 |
| 4-1992 | H | H | Br | A1 | J1 | Q98 |
| 4-1993 | H | H | CH₂F | A1 | J1 | Q98 |
| 4-1994 | H | H | CHF2 | A1 | J1 | Q98 |
| 4-1995 | H | H | Et | A1 | J1 | Q98 |
| 4-1996 | H | H | Pr | A1 | J1 | Q98 |
| 4-1997 | H | H | cPr | A1 | J1 | Q98 |
| 4-1998 | H | H | ethynyl | A1 | J1 | Q98 |
| 4-1999 | F | H | Me | A1 | J1 | Q98 |
| 4-2000 | F | F | Me | A1 | J1 | Q98 |
| 4-2001 | Me | H | Me | A1 | J1 | Q98 |
| 4-2002 | Me | Me | Me | A1 | J1 | Q98 |
| 4-2003 | H | H | H | A2 | J1 | Q98 |
| 4-2004 | H | H | Cl | A2 | J1 | Q98 |
| 4-2005 | H | H | Br | A2 | J1 | Q98 |
| 4-2006 | H | H | CH₂F | A2 | J1 | Q98 |
| 4-2007 | H | H | CHF2 | A2 | J1 | Q98 |
| 4-2008 | H | H | Et | A2 | J1 | Q98 |
| 4-2009 | H | H | Pr | A2 | J1 | Q98 |
| 4-2010 | H | H | cPr | A2 | J1 | Q98 |
| 4-2011 | H | H | ethynyl | A2 | J1 | Q98 |
| 4-2012 | F | H | Me | A2 | J1 | Q98 |
| 4-2013 | F | F | Me | A2 | J1 | Q98 |
| 4-2014 | Me | H | Me | A2 | J1 | Q98 |
| 4-2015 | Me | Me | Me | A2 | J1 | Q98 |
| 4-2016 | H | H | H | A6 | J1 | Q98 |
| 4-2017 | H | H | Cl | A6 | J1 | Q98 |
| 4-2018 | H | H | Br | A6 | J1 | Q98 |
| 4-2019 | H | H | CH₂F | A6 | J1 | Q98 |
| 4-2020 | H | H | CHF2 | A6 | J1 | Q98 |
| 4-2021 | H | H | Et | A6 | J1 | Q98 |
| 4-2022 | H | H | Pr | A6 | J1 | Q98 |
| 4-2023 | H | H | cPr | A6 | J1 | Q98 |
| 4-2024 | H | H | ethynyl | A6 | J1 | Q98 |
| 4-2025 | F | H | Me | A6 | J1 | Q98 |
| 4-2026 | F | F | Me | A6 | J1 | Q98 |
| 4-2027 | Me | H | Me | A6 | J1 | Q98 |
| 4-2028 | Me | Me | Me | A6 | J1 | Q98 |
| 4-2029 | H | H | H | A9 | J1 | Q98 |
| 4-2030 | H | H | Cl | A9 | J1 | Q98 |
| 4-2031 | H | H | Br | A9 | J1 | Q98 |
| 4-2032 | H | H | CH₂F | A9 | J1 | Q98 |
| 4-2033 | H | H | CHF2 | A9 | J1 | Q98 |
| 4-2034 | H | H | Et | A9 | J1 | Q98 |
| 4-2035 | H | H | Pr | A9 | J1 | Q98 |
| 4-2036 | H | H | cPr | A9 | J1 | Q98 |
| 4-2037 | H | H | ethynyl | A9 | J1 | Q98 |
| 4-2038 | F | H | Me | A9 | J1 | Q98 |
| 4-2039 | F | F | Me | A9 | J1 | Q98 |
| 4-2040 | Me | H | Me | A9 | J1 | Q98 |
| 4-2041 | Me | Me | Me | A9 | J1 | Q98 |
| 4-2042 | H | H | H | A11 | J1 | Q98 |
| 4-2043 | H | H | Cl | A11 | J1 | Q98 |
| 4-2044 | H | H | Br | A11 | J1 | Q98 |
| 4-2045 | H | H | CH₂F | A11 | J1 | Q98 |
| 4-2046 | H | H | CHF2 | A11 | J1 | Q98 |
| 4-2047 | H | H | Et | A11 | J1 | Q98 |
| 4-2048 | H | H | Pr | A11 | J1 | Q98 |
| 4-2049 | H | H | cPr | A11 | J1 | Q98 |
| 4-2050 | H | H | ethynyl | A11 | J1 | Q98 |
| 4-2051 | F | H | Me | A11 | J1 | Q98 |
| 4-2052 | F | F | Me | A11 | J1 | Q98 |
| 4-2053 | Me | H | Me | A11 | J1 | Q98 |
| 4-2054 | Me | Me | Me | A11 | J1 | Q98 |
| 4-2055 | H | H | H | A12 | J1 | Q98 |
| 4-2056 | H | H | Cl | A12 | J1 | Q98 |
| 4-2057 | H | H | Br | A12 | J1 | Q98 |
| 4-2058 | H | H | CH₂F | A12 | J1 | Q98 |
| 4-2059 | H | H | CHF2 | A12 | J1 | Q98 |
| 4-2060 | H | H | Et | A12 | J1 | Q98 |
| 4-2061 | H | H | Pr | A12 | J1 | Q98 |
| 4-2062 | H | H | cPr | A12 | J1 | Q98 |
| 4-2063 | H | H | ethynyl | A12 | J1 | Q98 |
| 4-2064 | F | H | Me | A12 | J1 | Q98 |
| 4-2065 | F | F | Me | A12 | J1 | Q98 |
| 4-2066 | Me | H | Me | A12 | J1 | Q98 |
| 4-2067 | Me | Me | Me | A12 | J1 | Q98 |
| 4-2068 | H | H | H | A19 | J1 | Q98 |
| 4-2069 | H | H | Cl | A19 | J1 | Q98 |
| 4-2070 | H | H | Br | A19 | J1 | Q98 |
| 4-2071 | H | H | CH₂F | A19 | J1 | Q98 |
| 4-2072 | H | H | CHF2 | A19 | J1 | Q98 |
| 4-2073 | H | H | Et | A19 | J1 | Q98 |
| 4-2074 | H | H | Pr | A19 | J1 | Q98 |
| 4-2075 | H | H | cPr | A19 | J1 | Q98 |
| 4-2076 | H | H | ethynyl | A19 | J1 | Q98 |
| 4-2077 | F | H | Me | A19 | J1 | Q98 |
| 4-2078 | F | F | Me | A19 | J1 | Q98 |
| 4-2079 | Me | H | Me | A19 | J1 | Q98 |
| 4-2080 | Me | Me | Me | A19 | J1 | Q98 |
| 4-2081 | H | H | H | A20 | J1 | Q98 |
| 4-2082 | H | H | Cl | A20 | J1 | Q98 |
| 4-2083 | H | H | Br | A20 | J1 | Q98 |
| 4-2084 | H | H | CH₂F | A20 | J1 | Q98 |
| 4-2085 | H | H | CHF2 | A20 | J1 | Q98 |
| 4-2086 | H | H | Et | A20 | J1 | Q98 |
| 4-2087 | H | H | Pr | A20 | J1 | Q98 |
| 4-2088 | H | H | cPr | A20 | J1 | Q98 |
| 4-2089 | H | H | ethynyl | A20 | J1 | Q98 |
| 4-2090 | F | H | Me | A20 | J1 | Q98 |
| 4-2091 | F | F | Me | A20 | J1 | Q98 |
| 4-2092 | Me | H | Me | A20 | J1 | Q98 |
| 4-2093 | Me | Me | Me | A20 | J1 | Q98 |
| 4-2094 | H | H | H | A21 | J1 | Q98 |
| 4-2095 | H | H | Cl | A21 | J1 | Q98 |
| 4-2096 | H | H | Br | A21 | J1 | Q98 |
| 4-2097 | H | H | CH₂F | A21 | J1 | Q98 |
| 4-2098 | H | H | CHF2 | A21 | J1 | Q98 |
| 4-2099 | H | H | Et | A21 | J1 | Q98 |
| 4-2100 | H | H | Pr | A21 | J1 | Q98 |
| 4-2101 | H | H | cPr | A21 | J1 | Q98 |
| 4-2102 | H | H | ethynyl | A21 | J1 | Q98 |
| 4-2103 | F | H | Me | A21 | J1 | Q98 |
| 4-2104 | F | F | Me | A21 | J1 | Q98 |
| 4-2105 | Me | H | Me | A21 | J1 | Q98 |
| 4-2106 | Me | Me | Me | A21 | J1 | Q98 |
| 4-2107 | H | H | H | A22 | J1 | Q98 |
| 4-2108 | H | H | Cl | A22 | J1 | Q98 |
| 4-2109 | H | H | Br | A22 | J1 | Q98 |
| 4-2110 | H | H | CH₂F | A22 | J1 | Q98 |
| 4-2111 | H | H | CHF2 | A22 | J1 | Q98 |
| 4-2112 | H | H | Et | A22 | J1 | Q98 |
| 4-2113 | H | H | Pr | A22 | J1 | Q98 |
| 4-2114 | H | H | cPr | A22 | J1 | Q98 |
| 4-2115 | H | H | ethynyl | A22 | J1 | Q98 |
| 4-2116 | F | H | Me | A22 | J1 | Q98 |
| 4-2117 | F | F | Me | A22 | J1 | Q98 |
| 4-2118 | Me | H | Me | A22 | J1 | Q98 |
| 4-2119 | Me | Me | Me | A22 | J1 | Q98 |
| 4-2120 | H | H | H | A26 | J1 | Q98 |
| 4-2121 | H | H | Cl | A26 | J1 | Q98 |
| 4-2122 | H | H | Br | A26 | J1 | Q98 |
| 4-2123 | H | H | CH₂F | A26 | J1 | Q98 |
| 4-2124 | H | H | CHF2 | A26 | J1 | Q98 |
| 4-2125 | H | H | Et | A26 | J1 | Q98 |
| 4-2126 | H | H | Pr | A26 | J1 | Q98 |
| 4-2127 | H | H | cPr | A26 | J1 | Q98 |
| 4-2128 | H | H | ethynyl | A26 | J1 | Q98 |
| 4-2129 | F | H | Me | A26 | J1 | Q98 |
| 4-2130 | F | F | Me | A26 | J1 | Q98 |
| 4-2131 | Me | H | Me | A26 | J1 | Q98 |
| 4-2132 | Me | Me | Me | A26 | J1 | Q98 |
| 4-2133 | H | H | H | A27 | J1 | Q98 |
| 4-2134 | H | H | Cl | A27 | J1 | Q98 |
| 4-2135 | H | H | Br | A27 | J1 | Q98 |
| 4-2136 | H | H | CH₂F | A27 | J1 | Q98 |
| 4-2137 | H | H | CHF2 | A27 | J1 | Q98 |
| 4-2138 | H | H | Et | A27 | J1 | Q98 |
| 4-2139 | H | H | Pr | A27 | J1 | Q98 |
| 4-2140 | H | H | cPr | A27 | J1 | Q98 |
| 4-2141 | H | H | ethynyl | A27 | J1 | Q98 |
| 4-2142 | F | H | Me | A27 | J1 | Q98 |
| 4-2143 | F | F | Me | A27 | J1 | Q98 |
| 4-2144 | Me | H | Me | A27 | J1 | Q98 |
| 4-2145 | Me | Me | Me | A27 | J1 | Q98 |
| 4-2146 | H | H | H | A28 | J1 | Q98 |
| 4-2147 | H | H | Cl | A28 | J1 | Q98 |
| 4-2148 | H | H | Br | A28 | J1 | Q98 |
| 4-2149 | H | H | CH₂F | A28 | J1 | Q98 |
| 4-2150 | H | H | CHF2 | A28 | J1 | Q98 |
| 4-2151 | H | H | Et | A28 | J1 | Q98 |
| 4-2152 | H | H | Pr | A28 | J1 | Q98 |
| 4-2153 | H | H | cPr | A28 | J1 | Q98 |
| 4-2154 | H | H | ethynyl | A28 | J1 | Q98 |
| 4-2155 | F | H | Me | A28 | J1 | Q98 |
| 4-2156 | F | F | Me | A28 | J1 | Q98 |
| 4-2157 | Me | H | Me | A28 | J1 | Q98 |
| 4-2158 | Me | Me | Me | A28 | J1 | Q98 |
| 4-2159 | H | H | H | A29 | J1 | Q98 |
| 4-2160 | H | H | Cl | A29 | J1 | Q98 |
| 4-2161 | H | H | Br | A29 | J1 | Q98 |
| 4-2162 | H | H | CH₂F | A29 | J1 | Q98 |
| 4-2163 | H | H | CHF2 | A29 | J1 | Q98 |
| 4-2164 | H | H | Et | A29 | J1 | Q98 |
| 4-2165 | H | H | Pr | A29 | J1 | Q98 |
| 4-2166 | H | H | cPr | A29 | J1 | Q98 |
| 4-2167 | H | H | ethynyl | A29 | J1 | Q98 |
| 4-2168 | F | H | Me | A29 | J1 | Q98 |
| 4-2169 | F | F | Me | A29 | J1 | Q98 |
| 4-2170 | Me | H | Me | A29 | J1 | Q98 |
| 4-2171 | Me | Me | Me | A29 | J1 | Q98 |
| 4-2172 | H | H | H | A30 | J1 | Q98 |
| 4-2173 | H | H | Cl | A30 | J1 | Q98 |
| 4-2174 | H | H | Br | A30 | J1 | Q98 |
| 4-2175 | H | H | CH₂F | A30 | J1 | Q98 |
| 4-2176 | H | H | CHF2 | A30 | J1 | Q98 |
| 4-2177 | H | H | Et | A30 | J1 | Q98 |
| 4-2178 | H | H | Pr | A30 | J1 | Q98 |
| 4-2179 | H | H | cPr | A30 | J1 | Q98 |
| 4-2180 | H | H | ethynyl | A30 | J1 | Q98 |
| 4-2181 | F | H | Me | A30 | J1 | Q98 |
| 4-2182 | F | F | Me | A30 | J1 | Q98 |
| 4-2183 | Me | H | Me | A30 | J1 | Q98 |
| 4-2184 | Me | Me | Me | A30 | J1 | Q98 |
| 4-2185 | H | H | H | A31 | J1 | Q98 |
| 4-2186 | H | H | Cl | A31 | J1 | Q98 |
| 4-2187 | H | H | Br | A31 | J1 | Q98 |
| 4-2188 | H | H | CH₂F | A31 | J1 | Q98 |
| 4-2189 | H | H | CHF2 | A31 | J1 | Q98 |
| 4-2190 | H | H | Et | A31 | J1 | Q98 |
| 4-2191 | H | H | Pr | A31 | J1 | Q98 |
| 4-2192 | H | H | cPr | A31 | J1 | Q98 |
| 4-2193 | H | H | ethynyl | A31 | J1 | Q98 |
| 4-2194 | F | H | Me | A31 | J1 | Q98 |
| 4-2195 | F | F | Me | A31 | J1 | Q98 |
| 4-2196 | Me | H | Me | A31 | J1 | Q98 |
| 4-2197 | Me | Me | Me | A31 | J1 | Q98 |
| 4-2198 | H | H | H | A51 | J1 | Q98 |
| 4-2199 | H | H | Cl | A51 | J1 | Q98 |
| 4-2200 | H | H | Br | A51 | J1 | Q98 |
| 4-2201 | H | H | CH₂F | A51 | J1 | Q98 |
| 4-2202 | H | H | CHF2 | A51 | J1 | Q98 |
| 4-2203 | H | H | Et | A51 | J1 | Q98 |
| 4-2204 | H | H | Pr | A51 | J1 | Q98 |
| 4-2205 | H | H | cPr | A51 | J1 | Q98 |
| 4-2206 | H | H | ethynyl | A51 | J1 | Q98 |
| 4-2207 | F | H | Me | A51 | J1 | Q98 |
| 4-2208 | F | F | Me | A51 | J1 | Q98 |
| 4-2209 | Me | H | Me | A51 | J1 | Q98 |
| 4-2210 | Me | Me | Me | A51 | J1 | Q98 |
| 4-2211 | H | H | H | A1 | J2 | Q106 |
| 4-2212 | H | H | Cl | A1 | J2 | Q106 |
| 4-2213 | H | H | Br | A1 | J2 | Q106 |
| 4-2214 | H | H | CH₂F | A1 | J2 | Q106 |
| 4-2215 | H | H | CHF2 | A1 | J2 | Q106 |
| 4-2216 | H | H | Et | A1 | J2 | Q106 |
| 4-2217 | H | H | Pr | A1 | J2 | Q106 |
| 4-2218 | H | H | cPr | A1 | J2 | Q106 |
| 4-2219 | H | H | ethynyl | A1 | J2 | Q106 |
| 4-2220 | F | H | Me | A1 | J2 | Q106 |
| 4-2221 | F | F | Me | A1 | J2 | Q106 |
| 4-2222 | Me | H | Me | A1 | J2 | Q106 |
| 4-2223 | Me | Me | Me | A1 | J2 | Q106 |
| 4-2224 | H | H | H | A2 | J2 | Q106 |
| 4-2225 | H | H | Cl | A2 | J2 | Q106 |
| 4-2226 | H | H | Br | A2 | J2 | Q106 |
| 4-2227 | H | H | CH₂F | A2 | J2 | Q106 |
| 4-2228 | H | H | CHF2 | A2 | J2 | Q106 |
| 4-2229 | H | H | Et | A2 | J2 | Q106 |
| 4-2230 | H | H | Pr | A2 | J2 | Q106 |
| 4-2231 | H | H | cPr | A2 | J2 | Q106 |
| 4-2232 | H | H | ethynyl | A2 | J2 | Q106 |
| 4-2233 | F | H | Me | A2 | J2 | Q106 |
| 4-2234 | F | F | Me | A2 | J2 | Q106 |
| 4-2235 | Me | H | Me | A2 | J2 | Q106 |
| 4-2236 | Me | Me | Me | A2 | J2 | Q106 |
| 4-2237 | H | H | H | A6 | J2 | Q106 |
| 4-2238 | H | H | Cl | A6 | J2 | Q106 |
| 4-2239 | H | H | Br | A6 | J2 | Q106 |
| 4-2240 | H | H | CH₂F | A6 | J2 | Q106 |
| 4-2241 | H | H | CHF2 | A6 | J2 | Q106 |
| 4-2242 | H | H | Et | A6 | J2 | Q106 |
| 4-2243 | H | H | Pr | A6 | J2 | Q106 |
| 4-2244 | H | H | cPr | A6 | J2 | Q106 |
| 4-2245 | H | H | ethynyl | A6 | J2 | Q106 |
| 4-2246 | F | H | Me | A6 | J2 | Q106 |
| 4-2247 | F | F | Me | A6 | J2 | Q106 |
| 4-2248 | Me | H | Me | A6 | J2 | Q106 |
| 4-2249 | Me | Me | Me | A6 | J2 | Q106 |
| 4-2250 | H | H | H | A9 | J2 | Q106 |
| 4-2251 | H | H | Cl | A9 | J2 | Q106 |
| 4-2252 | H | H | Br | A9 | J2 | Q106 |
| 4-2253 | H | H | CH₂F | A9 | J2 | Q106 |
| 4-2254 | H | H | CHF2 | A9 | J2 | Q106 |
| 4-2255 | H | H | Et | A9 | J2 | Q106 |
| 4-2256 | H | H | Pr | A9 | J2 | Q106 |
| 4-2257 | H | H | cPr | A9 | J2 | Q106 |
| 4-2258 | H | H | ethynyl | A9 | J2 | Q106 |
| 4-2259 | F | H | Me | A9 | J2 | Q106 |
| 4-2260 | F | F | Me | A9 | J2 | Q106 |
| 4-2261 | Me | H | Me | A9 | J2 | Q106 |
| 4-2262 | Me | Me | Me | A9 | J2 | Q106 |
| 4-2263 | H | H | H | A11 | J2 | Q106 |
| 4-2264 | H | H | Cl | A11 | J2 | Q106 |
| 4-2265 | H | H | Br | A11 | J2 | Q106 |
| 4-2266 | H | H | CH₂F | A11 | J2 | Q106 |
| 4-2267 | H | H | CHF2 | A11 | J2 | Q106 |
| 4-2268 | H | H | Et | A11 | J2 | Q106 |
| 4-2269 | H | H | Pr | A11 | J2 | Q106 |
| 4-2270 | H | H | cPr | A11 | J2 | Q106 |
| 4-2271 | H | H | ethynyl | A11 | J2 | Q106 |
| 4-2272 | F | H | Me | A11 | J2 | Q106 |
| 4-2273 | F | F | Me | A11 | J2 | Q106 |
| 4-2274 | Me | H | Me | A11 | J2 | Q106 |
| 4-2275 | Me | Me | Me | A11 | J2 | Q106 |
| 4-2276 | H | H | H | A12 | J2 | Q106 |
| 4-2277 | H | H | Cl | A12 | J2 | Q106 |
| 4-2278 | H | H | Br | A12 | J2 | Q106 |
| 4-2279 | H | H | CH₂F | A12 | J2 | Q106 |
| 4-2280 | H | H | CHF2 | A12 | J2 | Q106 |
| 4-2281 | H | H | Et | A12 | J2 | Q106 |
| 4-2282 | H | H | Pr | A12 | J2 | Q106 |
| 4-2283 | H | H | cPr | A12 | J2 | Q106 |
| 4-2284 | H | H | ethynyl | A12 | J2 | Q106 |
| 4-2285 | F | H | Me | A12 | J2 | Q106 |
| 4-2286 | F | F | Me | A12 | J2 | Q106 |
| 4-2287 | Me | H | Me | A12 | J2 | Q106 |
| 4-2288 | Me | Me | Me | A12 | J2 | Q106 |
| 4-2289 | H | H | H | A19 | J2 | Q106 |
| 4-2290 | H | H | Cl | A19 | J2 | Q106 |
| 4-2291 | H | H | Br | A19 | J2 | Q106 |
| 4-2292 | H | H | CH₂F | A19 | J2 | Q106 |
| 4-2293 | H | H | CHF2 | A19 | J2 | Q106 |
| 4-2294 | H | H | Et | A19 | J2 | Q106 |
| 4-2295 | H | H | Pr | A19 | J2 | Q106 |
| 4-2296 | H | H | cPr | A19 | J2 | Q106 |
| 4-2297 | H | H | ethynyl | A19 | J2 | Q106 |
| 4-2298 | F | H | Me | A19 | J2 | Q106 |
| 4-2299 | F | F | Me | A19 | J2 | Q106 |
| 4-2300 | Me | H | Me | A19 | J2 | Q106 |
| 4-2301 | Me | Me | Me | A19 | J2 | Q106 |
| 4-2302 | H | H | H | A20 | J2 | Q106 |
| 4-2303 | H | H | Cl | A20 | J2 | Q106 |
| 4-2304 | H | H | Br | A20 | J2 | Q106 |
| 4-2305 | H | H | CH₂F | A20 | J2 | Q106 |
| 4-2306 | H | H | CHF2 | A20 | J2 | Q106 |
| 4-2307 | H | H | Et | A20 | J2 | Q106 |
| 4-2308 | H | H | Pr | A20 | J2 | Q106 |
| 4-2309 | H | H | cPr | A20 | J2 | Q106 |
| 4-2310 | H | H | ethynyl | A20 | J2 | Q106 |
| 4-2311 | F | H | Me | A20 | J2 | Q106 |
| 4-2312 | F | F | Me | A20 | J2 | Q106 |
| 4-2313 | Me | H | Me | A20 | J2 | Q106 |
| 4-2314 | Me | Me | Me | A20 | J2 | Q106 |
| 4-2315 | H | H | H | A21 | J2 | Q106 |
| 4-2316 | H | H | Cl | A21 | J2 | Q106 |
| 4-2317 | H | H | Br | A21 | J2 | Q106 |
| 4-2318 | H | H | CH₂F | A21 | J2 | Q106 |
| 4-2319 | H | H | CHF2 | A21 | J2 | Q106 |
| 4-2320 | H | H | Et | A21 | J2 | Q106 |
| 4-2321 | H | H | Pr | A21 | J2 | Q106 |
| 4-2322 | H | H | cPr | A21 | J2 | Q106 |
| 4-2323 | H | H | ethynyl | A21 | J2 | Q106 |
| 4-2324 | F | H | Me | A21 | J2 | Q106 |
| 4-2325 | F | F | Me | A21 | J2 | Q106 |
| 4-2326 | Me | H | Me | A21 | J2 | Q106 |
| 4-2327 | Me | Me | Me | A21 | J2 | Q106 |
| 4-2328 | H | H | H | A22 | J2 | Q106 |
| 4-2329 | H | H | Cl | A22 | J2 | Q106 |
| 4-2330 | H | H | Br | A22 | J2 | Q106 |
| 4-2331 | H | H | CH₂F | A22 | J2 | Q106 |
| 4-2332 | H | H | CHF2 | A22 | J2 | Q106 |
| 4-2333 | H | H | Et | A22 | J2 | Q106 |
| 4-2334 | H | H | Pr | A22 | J2 | Q106 |
| 4-2335 | H | H | cPr | A22 | J2 | Q106 |
| 4-2336 | H | H | ethynyl | A22 | J2 | Q106 |
| 4-2337 | F | H | Me | A22 | J2 | Q106 |
| 4-2338 | F | F | Me | A22 | J2 | Q106 |
| 4-2339 | Me | H | Me | A22 | J2 | Q106 |
| 4-2340 | Me | Me | Me | A22 | J2 | Q106 |
| 4-2341 | H | H | H | A26 | J2 | Q106 |
| 4-2342 | H | H | Cl | A26 | J2 | Q106 |
| 4-2343 | H | H | Br | A26 | J2 | Q106 |
| 4-2344 | H | H | CH₂F | A26 | J2 | Q106 |
| 4-2345 | H | H | CHF2 | A26 | J2 | Q106 |
| 4-2346 | H | H | Et | A26 | J2 | Q106 |
| 4-2347 | H | H | Pr | A26 | J2 | Q106 |
| 4-2348 | H | H | cPr | A26 | J2 | Q106 |
| 4-2349 | H | H | ethynyl | A26 | J2 | Q106 |
| 4-2350 | F | H | Me | A26 | J2 | Q106 |
| 4-2351 | F | F | Me | A26 | J2 | Q106 |
| 4-2352 | Me | H | Me | A26 | J2 | Q106 |
| 4-2353 | Me | Me | Me | A26 | J2 | Q106 |
| 4-2354 | H | H | H | A27 | J2 | Q106 |
| 4-2355 | H | H | Cl | A27 | J2 | Q106 |
| 4-2356 | H | H | Br | A27 | J2 | Q106 |
| 4-2357 | H | H | CH₂F | A27 | J2 | Q106 |
| 4-2358 | H | H | CHF2 | A27 | J2 | Q106 |
| 4-2359 | H | H | Et | A27 | J2 | Q106 |
| 4-2360 | H | H | Pr | A27 | J2 | Q106 |
| 4-2361 | H | H | cPr | A27 | J2 | Q106 |
| 4-2362 | H | H | ethynyl | A27 | J2 | Q106 |
| 4-2363 | F | H | Me | A27 | J2 | Q106 |
| 4-2364 | F | F | Me | A27 | J2 | Q106 |
| 4-2365 | Me | H | Me | A27 | J2 | Q106 |
| 4-2366 | Me | Me | Me | A27 | J2 | Q106 |
| 4-2367 | H | H | H | A28 | J2 | Q106 |
| 4-2368 | H | H | Cl | A28 | J2 | Q106 |
| 4-2369 | H | H | Br | A28 | J2 | Q106 |
| 4-2370 | H | H | CH₂F | A28 | J2 | Q106 |
| 4-2371 | H | H | CHF2 | A28 | J2 | Q106 |
| 4-2372 | H | H | Et | A28 | J2 | Q106 |
| 4-2373 | H | H | Pr | A28 | J2 | Q106 |
| 4-2374 | H | H | cPr | A28 | J2 | Q106 |
| 4-2375 | H | H | ethynyl | A28 | J2 | Q106 |
| 4-2376 | F | H | Me | A28 | J2 | Q106 |
| 4-2377 | F | F | Me | A28 | J2 | Q106 |
| 4-2378 | Me | H | Me | A28 | J2 | Q106 |
| 4-2379 | Me | Me | Me | A28 | J2 | Q106 |
| 4-2380 | H | H | H | A29 | J2 | Q106 |
| 4-2381 | H | H | Cl | A29 | J2 | Q106 |
| 4-2382 | H | H | Br | A29 | J2 | Q106 |
| 4-2383 | H | H | CH₂F | A29 | J2 | Q106 |
| 4-2384 | H | H | CHF2 | A29 | J2 | Q106 |
| 4-2385 | H | H | Et | A29 | J2 | Q106 |
| 4-2386 | H | H | Pr | A29 | J2 | Q106 |
| 4-2387 | H | H | cPr | A29 | J2 | Q106 |
| 4-2388 | H | H | ethynyl | A29 | J2 | Q106 |
| 4-2389 | F | H | Me | A29 | J2 | Q106 |
| 4-2390 | F | F | Me | A29 | J2 | Q106 |
| 4-2391 | Me | H | Me | A29 | J2 | Q106 |
| 4-2392 | Me | Me | Me | A29 | J2 | Q106 |
| 4-2393 | H | H | H | A30 | J2 | Q106 |
| 4-2394 | H | H | Cl | A30 | J2 | Q106 |
| 4-2395 | H | H | Br | A30 | J2 | Q106 |
| 4-2396 | H | H | CH₂F | A30 | J2 | Q106 |
| 4-2397 | H | H | CHF2 | A30 | J2 | Q106 |
| 4-2398 | H | H | Et | A30 | J2 | Q106 |
| 4-2399 | H | H | Pr | A30 | J2 | Q106 |
| 4-2400 | H | H | cPr | A30 | J2 | Q106 |
| 4-2401 | H | H | ethynyl | A30 | J2 | Q106 |
| 4-2402 | F | H | Me | A30 | J2 | Q106 |
| 4-2403 | F | F | Me | A30 | J2 | Q106 |
| 4-2404 | Me | H | Me | A30 | J2 | Q106 |
| 4-2405 | Me | Me | Me | A30 | J2 | Q106 |
| 4-2406 | H | H | H | A31 | J2 | Q106 |
| 4-2407 | H | H | Cl | A31 | J2 | Q106 |
| 4-2408 | H | H | Br | A31 | J2 | Q106 |
| 4-2409 | H | H | CH₂F | A31 | J2 | Q106 |
| 4-2410 | H | H | CHF2 | A31 | J2 | Q106 |
| 4-2411 | H | H | Et | A31 | J2 | Q106 |
| 4-2412 | H | H | Pr | A31 | J2 | Q106 |
| 4-2413 | H | H | cPr | A31 | J2 | Q106 |
| 4-2414 | H | H | ethynyl | A31 | J2 | Q106 |
| 4-2415 | F | H | Me | A31 | J2 | Q106 |
| 4-2416 | F | F | Me | A31 | J2 | Q106 |
| 4-2417 | Me | H | Me | A31 | J2 | Q106 |
| 4-2418 | Me | Me | Me | A31 | J2 | Q106 |
| 4-2419 | H | H | H | A51 | J2 | Q106 |
| 4-2420 | H | H | Cl | A51 | J2 | Q106 |
| 4-2421 | H | H | Br | A51 | J2 | Q106 |
| 4-2422 | H | H | CH₂F | A51 | J2 | Q106 |
| 4-2423 | H | H | CHF2 | A51 | J2 | Q106 |
| 4-2424 | H | H | Et | A51 | J2 | Q106 |
| 4-2425 | H | H | Pr | A51 | J2 | Q106 |
| 4-2426 | H | H | cPr | A51 | J2 | Q106 |
| 4-2427 | H | H | ethynyl | A51 | J2 | Q106 |
| 4-2428 | F | H | Me | A51 | J2 | Q106 |
| 4-2429 | F | F | Me | A51 | J2 | Q106 |
| 4-2430 | Me | H | Me | A51 | J2 | Q106 |
| 4-2431 | Me | Me | Me | A51 | J2 | Q106 |
| 4-2432 | H | H | H | A1 | J1 | Q106 |
| 4-2433 | H | H | Cl | A1 | J1 | Q106 |
| 4-2434 | H | H | Br | A1 | J1 | Q106 |
| 4-2435 | H | H | CH₂F | A1 | J1 | Q106 |
| 4-2436 | H | H | CHF2 | A1 | J1 | Q106 |
| 4-2437 | H | H | Et | A1 | J1 | Q106 |
| 4-2438 | H | H | Pr | A1 | J1 | Q106 |
| 4-2439 | H | H | cPr | A1 | J1 | Q106 |
| 4-2440 | H | H | ethynyl | A1 | J1 | Q106 |
| 4-2441 | F | H | Me | A1 | J1 | Q106 |
| 4-2442 | F | F | Me | A1 | J1 | Q106 |
| 4-2443 | Me | H | Me | A1 | J1 | Q106 |
| 4-2444 | Me | Me | Me | A1 | J1 | Q106 |
| 4-2445 | H | H | H | A2 | J1 | Q106 |
| 4-2446 | H | H | Cl | A2 | J1 | Q106 |
| 4-2447 | H | H | Br | A2 | J1 | Q106 |
| 4-2448 | H | H | CH₂F | A2 | J1 | Q106 |
| 4-2449 | H | H | CHF2 | A2 | J1 | Q106 |
| 4-2450 | H | H | Et | A2 | J1 | Q106 |
| 4-2451 | H | H | Pr | A2 | J1 | Q106 |
| 4-2452 | H | H | cPr | A2 | J1 | Q106 |
| 4-2453 | H | H | ethynyl | A2 | J1 | Q106 |
| 4-2454 | F | H | Me | A2 | J1 | Q106 |
| 4-2455 | F | F | Me | A2 | J1 | Q106 |
| 4-2456 | Me | H | Me | A2 | J1 | Q106 |
| 4-2457 | Me | Me | Me | A2 | J1 | Q106 |
| 4-2458 | H | H | H | A6 | J1 | Q106 |
| 4-2459 | H | H | Cl | A6 | J1 | Q106 |
| 4-2460 | H | H | Br | A6 | J1 | Q106 |
| 4-2461 | H | H | CH₂F | A6 | J1 | Q106 |
| 4-2462 | H | H | CHF2 | A6 | J1 | Q106 |
| 4-2463 | H | H | Et | A6 | J1 | Q106 |
| 4-2464 | H | H | Pr | A6 | J1 | Q106 |
| 4-2465 | H | H | cPr | A6 | J1 | Q106 |
| 4-2466 | H | H | ethynyl | A6 | J1 | Q106 |
| 4-2467 | F | H | Me | A6 | J1 | Q106 |
| 4-2468 | F | F | Me | A6 | J1 | Q106 |
| 4-2469 | Me | H | Me | A6 | J1 | Q106 |
| 4-2470 | Me | Me | Me | A6 | J1 | Q106 |
| 4-2471 | H | H | H | A9 | J1 | Q106 |
| 4-2472 | H | H | Cl | A9 | J1 | Q106 |
| 4-2473 | H | H | Br | A9 | J1 | Q106 |
| 4-2474 | H | H | CH₂F | A9 | J1 | Q106 |
| 4-2475 | H | H | CHF2 | A9 | J1 | Q106 |
| 4-2476 | H | H | Et | A9 | J1 | Q106 |
| 4-2477 | H | H | Pr | A9 | J1 | Q106 |
| 4-2478 | H | H | cPr | A9 | J1 | Q106 |
| 4-2479 | H | H | ethynyl | A9 | J1 | Q106 |
| 4-2480 | F | H | Me | A9 | J1 | Q106 |
| 4-2481 | F | F | Me | A9 | J1 | Q106 |
| 4-2482 | Me | H | Me | A9 | J1 | Q106 |
| 4-2483 | Me | Me | Me | A9 | J1 | Q106 |
| 4-2484 | H | H | H | A11 | J1 | Q106 |
| 4-2485 | H | H | Cl | A11 | J1 | Q106 |
| 4-2486 | H | H | Br | A11 | J1 | Q106 |
| 4-2487 | H | H | CH₂F | A11 | J1 | Q106 |
| 4-2488 | H | H | CHF2 | A11 | J1 | Q106 |
| 4-2489 | H | H | Et | A11 | J1 | Q106 |
| 4-2490 | H | H | Pr | A11 | J1 | Q106 |
| 4-2491 | H | H | cPr | A11 | J1 | Q106 |
| 4-2492 | H | H | ethynyl | A11 | J1 | Q106 |
| 4-2493 | F | H | Me | A11 | J1 | Q106 |
| 4-2494 | F | F | Me | A11 | J1 | Q106 |
| 4-2495 | Me | H | Me | A11 | J1 | Q106 |
| 4-2496 | Me | Me | Me | A11 | J1 | Q106 |
| 4-2497 | H | H | H | A12 | J1 | Q106 |
| 4-2498 | H | H | Cl | A12 | J1 | Q106 |
| 4-2499 | H | H | Br | A12 | J1 | Q106 |
| 4-2500 | H | H | CH₂F | A12 | J1 | Q106 |
| 4-2501 | H | H | CHF2 | A12 | J1 | Q106 |
| 4-2502 | H | H | Et | A12 | J1 | Q106 |
| 4-2503 | H | H | Pr | A12 | J1 | Q106 |
| 4-2504 | H | H | cPr | A12 | J1 | Q106 |
| 4-2505 | H | H | ethynyl | A12 | J1 | Q106 |
| 4-2506 | F | H | Me | A12 | J1 | Q106 |
| 4-2507 | F | F | Me | A12 | J1 | Q106 |
| 4-2508 | Me | H | Me | A12 | J1 | Q106 |
| 4-2509 | Me | Me | Me | A12 | J1 | Q106 |
| 4-2510 | H | H | H | A19 | J1 | Q106 |
| 4-2511 | H | H | Cl | A19 | J1 | Q106 |
| 4-2512 | H | H | Br | A19 | J1 | Q106 |
| 4-2513 | H | H | CH₂F | A19 | J1 | Q106 |
| 4-2514 | H | H | CHF2 | A19 | J1 | Q106 |
| 4-2515 | H | H | Et | A19 | J1 | Q106 |
| 4-2516 | H | H | Pr | A19 | J1 | Q106 |
| 4-2517 | H | H | cPr | A19 | J1 | Q106 |
| 4-2518 | H | H | ethynyl | A19 | J1 | Q106 |
| 4-2519 | F | H | Me | A19 | J1 | Q106 |
| 4-2520 | F | F | Me | A19 | J1 | Q106 |
| 4-2521 | Me | H | Me | A19 | J1 | Q106 |
| 4-2522 | Me | Me | Me | A19 | J1 | Q106 |
| 4-2523 | H | H | H | A20 | J1 | Q106 |
| 4-2524 | H | H | Cl | A20 | J1 | Q106 |
| 4-2525 | H | H | Br | A20 | J1 | Q106 |
| 4-2526 | H | H | CH₂F | A20 | J1 | Q106 |
| 4-2527 | H | H | CHF2 | A20 | J1 | Q106 |
| 4-2528 | H | H | Et | A20 | J1 | Q106 |
| 4-2529 | H | H | Pr | A20 | J1 | Q106 |
| 4-2530 | H | H | cPr | A20 | J1 | Q106 |
| 4-2531 | H | H | ethynyl | A20 | J1 | Q106 |
| 4-2532 | F | H | Me | A20 | J1 | Q106 |
| 4-2533 | F | F | Me | A20 | J1 | Q106 |
| 4-2534 | Me | H | Me | A20 | J1 | Q106 |
| 4-2535 | Me | Me | Me | A20 | J1 | Q106 |
| 4-2536 | H | H | H | A21 | J1 | Q106 |
| 4-2537 | H | H | Cl | A21 | J1 | Q106 |
| 4-2538 | H | H | Br | A21 | J1 | Q106 |
| 4-2539 | H | H | CH₂F | A21 | J1 | Q106 |
| 4-2540 | H | H | CHF2 | A21 | J1 | Q106 |
| 4-2541 | H | H | Et | A21 | J1 | Q106 |
| 4-2542 | H | H | Pr | A21 | J1 | Q106 |
| 4-2543 | H | H | cPr | A21 | J1 | Q106 |
| 4-2544 | H | H | ethynyl | A21 | J1 | Q106 |
| 4-2545 | F | H | Me | A21 | J1 | Q106 |
| 4-2546 | F | F | Me | A21 | J1 | Q106 |
| 4-2547 | Me | H | Me | A21 | J1 | Q106 |
| 4-2548 | Me | Me | Me | A21 | J1 | Q106 |
| 4-2549 | H | H | H | A22 | J1 | Q106 |
| 4-2550 | H | H | Cl | A22 | J1 | Q106 |
| 4-2551 | H | H | Br | A22 | J1 | Q106 |
| 4-2552 | H | H | CH₂F | A22 | J1 | Q106 |
| 4-2553 | H | H | CHF2 | A22 | J1 | Q106 |
| 4-2554 | H | H | Et | A22 | J1 | Q106 |
| 4-2555 | H | H | Pr | A22 | J1 | Q106 |
| 4-2556 | H | H | cPr | A22 | J1 | Q106 |
| 4-2557 | H | H | ethynyl | A22 | J1 | Q106 |
| 4-2558 | F | H | Me | A22 | J1 | Q106 |
| 4-2559 | F | F | Me | A22 | J1 | Q106 |
| 4-2560 | Me | H | Me | A22 | J1 | Q106 |
| 4-2561 | Me | Me | Me | A22 | J1 | Q106 |
| 4-2562 | H | H | H | A26 | J1 | Q106 |
| 4-2563 | H | H | Cl | A26 | J1 | Q106 |
| 4-2564 | H | H | Br | A26 | J1 | Q106 |
| 4-2565 | H | H | CH₂F | A26 | J1 | Q106 |
| 4-2566 | H | H | CHF2 | A26 | J1 | Q106 |
| 4-2567 | H | H | Et | A26 | J1 | Q106 |
| 4-2568 | H | H | Pr | A26 | J1 | Q106 |
| 4-2569 | H | H | cPr | A26 | J1 | Q106 |
| 4-2570 | H | H | ethynyl | A26 | J1 | Q106 |
| 4-2571 | F | H | Me | A26 | J1 | Q106 |
| 4-2572 | F | F | Me | A26 | J1 | Q106 |
| 4-2573 | Me | H | Me | A26 | J1 | Q106 |
| 4-2574 | Me | Me | Me | A26 | J1 | Q106 |
| 4-2575 | H | H | H | A27 | J1 | Q106 |
| 4-2576 | H | H | Cl | A27 | J1 | Q106 |
| 4-2577 | H | H | Br | A27 | J1 | Q106 |
| 4-2578 | H | H | CH₂F | A27 | J1 | Q106 |
| 4-2579 | H | H | CHF2 | A27 | J1 | Q106 |
| 4-2580 | H | H | Et | A27 | J1 | Q106 |
| 4-2581 | H | H | Pr | A27 | J1 | Q106 |
| 4-2582 | H | H | cPr | A27 | J1 | Q106 |
| 4-2583 | H | H | ethynyl | A27 | J1 | Q106 |
| 4-2584 | F | H | Me | A27 | J1 | Q106 |
| 4-2585 | F | F | Me | A27 | J1 | Q106 |
| 4-2586 | Me | H | Me | A27 | J1 | Q106 |
| 4-2587 | Me | Me | Me | A27 | J1 | Q106 |
| 4-2588 | H | H | H | A28 | J1 | Q106 |
| 4-2589 | H | H | Cl | A28 | J1 | Q106 |
| 4-2590 | H | H | Br | A28 | J1 | Q106 |
| 4-2591 | H | H | CH₂F | A28 | J1 | Q106 |
| 4-2592 | H | H | CHF2 | A28 | J1 | Q106 |
| 4-2593 | H | H | Et | A28 | J1 | Q106 |
| 4-2594 | H | H | Pr | A28 | J1 | Q106 |
| 4-2595 | H | H | cPr | A28 | J1 | Q106 |
| 4-2596 | H | H | ethynyl | A28 | J1 | Q106 |
| 4-2597 | F | H | Me | A28 | J1 | Q106 |
| 4-2598 | F | F | Me | A28 | J1 | Q106 |
| 4-2599 | Me | H | Me | A28 | J1 | Q106 |
| 4-2600 | Me | Me | Me | A28 | J1 | Q106 |
| 4-2601 | H | H | H | A29 | J1 | Q106 |
| 4-2602 | H | H | Cl | A29 | J1 | Q106 |
| 4-2603 | H | H | Br | A29 | J1 | Q106 |
| 4-2604 | H | H | CH₂F | A29 | J1 | Q106 |
| 4-2605 | H | H | CHF2 | A29 | J1 | Q106 |
| 4-2606 | H | H | Et | A29 | J1 | Q106 |
| 4-2607 | H | H | Pr | A29 | J1 | Q106 |
| 4-2608 | H | H | cPr | A29 | J1 | Q106 |
| 4-2609 | H | H | ethynyl | A29 | J1 | Q106 |
| 4-2610 | F | H | Me | A29 | J1 | Q106 |
| 4-2611 | F | F | Me | A29 | J1 | Q106 |
| 4-2612 | Me | H | Me | A29 | J1 | Q106 |
| 4-2613 | Me | Me | Me | A29 | J1 | Q106 |
| 4-2614 | H | H | H | A30 | J1 | Q106 |
| 4-2615 | H | H | Cl | A30 | J1 | Q106 |
| 4-2616 | H | H | Br | A30 | J1 | Q106 |
| 4-2617 | H | H | CH₂F | A30 | J1 | Q106 |
| 4-2618 | H | H | CHF2 | A30 | J1 | Q106 |
| 4-2619 | H | H | Et | A30 | J1 | Q106 |
| 4-2620 | H | H | Pr | A30 | J1 | Q106 |
| 4-2621 | H | H | cPr | A30 | J1 | Q106 |
| 4-2622 | H | H | ethynyl | A30 | J1 | Q106 |
| 4-2623 | F | H | Me | A30 | J1 | Q106 |
| 4-2624 | F | F | Me | A30 | J1 | Q106 |
| 4-2625 | Me | H | Me | A30 | J1 | Q106 |
| 4-2626 | Me | Me | Me | A30 | J1 | Q106 |
| 4-2627 | H | H | H | A31 | J1 | Q106 |
| 4-2628 | H | H | Cl | A31 | J1 | Q106 |
| 4-2629 | H | H | Br | A31 | J1 | Q106 |
| 4-2630 | H | H | CH₂F | A31 | J1 | Q106 |
| 4-2631 | H | H | CHF2 | A31 | J1 | Q106 |
| 4-2632 | H | H | Et | A31 | J1 | Q106 |
| 4-2633 | H | H | Pr | A31 | J1 | Q106 |
| 4-2634 | H | H | cPr | A31 | J1 | Q106 |
| 4-2635 | H | H | ethynyl | A31 | J1 | Q106 |
| 4-2636 | F | H | Me | A31 | J1 | Q106 |
| 4-2637 | F | F | Me | A31 | J1 | Q106 |
| 4-2638 | Me | H | Me | A31 | J1 | Q106 |
| 4-2639 | Me | Me | Me | A31 | J1 | Q106 |
| 4-2640 | H | H | H | A51 | J1 | Q106 |
| 4-2641 | H | H | Cl | A51 | J1 | Q106 |
| 4-2642 | H | H | Br | A51 | J1 | Q106 |
| 4-2643 | H | H | CH₂F | A51 | J1 | Q106 |
| 4-2644 | H | H | CHF2 | A51 | J1 | Q106 |
| 4-2645 | H | H | Et | A51 | J1 | Q106 |
| 4-2646 | H | H | Pr | A51 | J1 | Q106 |
| 4-2647 | H | H | cPr | A51 | J1 | Q106 |
| 4-2648 | H | H | ethynyl | A51 | J1 | Q106 |
| 4-2649 | F | H | Me | A51 | J1 | Q106 |
| 4-2650 | F | F | Me | A51 | J1 | Q106 |
| 4-2651 | Me | H | Me | A51 | J1 | Q106 |
| 4-2652 | Me | Me | Me | A51 | J1 | Q106 |
| 4-2653 | H | H | H | A1 | J2 | Q124 |
| 4-2654 | H | H | Cl | A1 | J2 | Q124 |
| 4-2655 | H | H | Br | A1 | J2 | Q124 |
| 4-2656 | H | H | CH₂F | A1 | J2 | Q124 |
| 4-2657 | H | H | CHF2 | A1 | J2 | Q124 |
| 4-2658 | H | H | Et | A1 | J2 | Q124 |
| 4-2659 | H | H | Pr | A1 | J2 | Q124 |
| 4-2660 | H | H | cPr | A1 | J2 | Q124 |
| 4-2661 | H | H | ethynyl | A1 | J2 | Q124 |
| 4-2662 | F | H | Me | A1 | J2 | Q124 |
| 4-2663 | F | F | Me | A1 | J2 | Q124 |
| 4-2664 | Me | H | Me | A1 | J2 | Q124 |
| 4-2665 | Me | Me | Me | A1 | J2 | Q124 |
| 4-2666 | H | H | H | A2 | J2 | Q124 |
| 4-2667 | H | H | Cl | A2 | J2 | Q124 |
| 4-2668 | H | H | Br | A2 | J2 | Q124 |
| 4-2669 | H | H | CH₂F | A2 | J2 | Q124 |
| 4-2670 | H | H | CHF2 | A2 | J2 | Q124 |
| 4-2671 | H | H | Et | A2 | J2 | Q124 |
| 4-2672 | H | H | Pr | A2 | J2 | Q124 |
| 4-2673 | H | H | cPr | A2 | J2 | Q124 |
| 4-2674 | H | H | ethynyl | A2 | J2 | Q124 |
| 4-2675 | F | H | Me | A2 | J2 | Q124 |
| 4-2676 | F | F | Me | A2 | J2 | Q124 |
| 4-2677 | Me | H | Me | A2 | J2 | Q124 |
| 4-2678 | Me | Me | Me | A2 | J2 | Q124 |
| 4-2679 | H | H | H | A6 | J2 | Q124 |
| 4-2680 | H | H | Cl | A6 | J2 | Q124 |
| 4-2681 | H | H | Br | A6 | J2 | Q124 |
| 4-2682 | H | H | CH₂F | A6 | J2 | Q124 |
| 4-2683 | H | H | CHF2 | A6 | J2 | Q124 |
| 4-2684 | H | H | Et | A6 | J2 | Q124 |
| 4-2685 | H | H | Pr | A6 | J2 | Q124 |
| 4-2686 | H | H | cPr | A6 | J2 | Q124 |
| 4-2687 | H | H | ethynyl | A6 | J2 | Q124 |
| 4-2688 | F | H | Me | A6 | J2 | Q124 |
| 4-2689 | F | F | Me | A6 | J2 | Q124 |
| 4-2690 | Me | H | Me | A6 | J2 | Q124 |
| 4-2691 | Me | Me | Me | A6 | J2 | Q124 |
| 4-2692 | H | H | H | A9 | J2 | Q124 |
| 4-2693 | H | H | Cl | A9 | J2 | Q124 |
| 4-2694 | H | H | Br | A9 | J2 | Q124 |
| 4-2695 | H | H | CH₂F | A9 | J2 | Q124 |
| 4-2696 | H | H | CHF2 | A9 | J2 | Q124 |
| 4-2697 | H | H | Et | A9 | J2 | Q124 |
| 4-2698 | H | H | Pr | A9 | J2 | Q124 |
| 4-2699 | H | H | cPr | A9 | J2 | Q124 |
| 4-2700 | H | H | ethynyl | A9 | J2 | Q124 |
| 4-2701 | F | H | Me | A9 | J2 | Q124 |
| 4-2702 | F | F | Me | A9 | J2 | Q124 |
| 4-2703 | Me | H | Me | A9 | J2 | Q124 |
| 4-2704 | Me | Me | Me | A9 | J2 | Q124 |
| 4-2705 | H | H | H | A11 | J2 | Q124 |
| 4-2706 | H | H | Cl | A11 | J2 | Q124 |
| 4-2707 | H | H | Br | A11 | J2 | Q124 |
| 4-2708 | H | H | CH₂F | A11 | J2 | Q124 |
| 4-2709 | H | H | CHF2 | A11 | J2 | Q124 |
| 4-2710 | H | H | Et | A11 | J2 | Q124 |
| 4-2711 | H | H | Pr | A11 | J2 | Q124 |
| 4-2712 | H | H | cPr | A11 | J2 | Q124 |
| 4-2713 | H | H | ethynyl | A11 | J2 | Q124 |
| 4-2714 | F | H | Me | A11 | J2 | Q124 |
| 4-2715 | F | F | Me | A11 | J2 | Q124 |
| 4-2716 | Me | H | Me | A11 | J2 | Q124 |
| 4-2717 | Me | Me | Me | A11 | J2 | Q124 |
| 4-2718 | H | H | H | A12 | J2 | Q124 |
| 4-2719 | H | H | Cl | A12 | J2 | Q124 |
| 4-2720 | H | H | Br | A12 | J2 | Q124 |
| 4-2721 | H | H | CH₂F | A12 | J2 | Q124 |
| 4-2722 | H | H | CHF2 | A12 | J2 | Q124 |
| 4-2723 | H | H | Et | A12 | J2 | Q124 |
| 4-2724 | H | H | Pr | A12 | J2 | Q124 |
| 4-2725 | H | H | cPr | A12 | J2 | Q124 |
| 4-2726 | H | H | ethynyl | A12 | J2 | Q124 |
| 4-2727 | F | H | Me | A12 | J2 | Q124 |
| 4-2728 | F | F | Me | A12 | J2 | Q124 |
| 4-2729 | Me | H | Me | A12 | J2 | Q124 |
| 4-2730 | Me | Me | Me | A12 | J2 | Q124 |
| 4-2731 | H | H | H | A19 | J2 | Q124 |
| 4-2732 | H | H | Cl | A19 | J2 | Q124 |
| 4-2733 | H | H | Br | A19 | J2 | Q124 |
| 4-2734 | H | H | CH₂F | A19 | J2 | Q124 |
| 4-2735 | H | H | CHF2 | A19 | J2 | Q124 |
| 4-2736 | H | H | Et | A19 | J2 | Q124 |
| 4-2737 | H | H | Pr | A19 | J2 | Q124 |
| 4-2738 | H | H | cPr | A19 | J2 | Q124 |
| 4-2739 | H | H | ethynyl | A19 | J2 | Q124 |
| 4-2740 | F | H | Me | A19 | J2 | Q124 |
| 4-2741 | F | F | Me | A19 | J2 | Q124 |
| 4-2742 | Me | H | Me | A19 | J2 | Q124 |
| 4-2743 | Me | Me | Me | A19 | J2 | Q124 |
| 4-2744 | H | H | H | A20 | J2 | Q124 |
| 4-2745 | H | H | Cl | A20 | J2 | Q124 |
| 4-2746 | H | H | Br | A20 | J2 | Q124 |
| 4-2747 | H | H | CH₂F | A20 | J2 | Q124 |
| 4-2748 | H | H | CHF2 | A20 | J2 | Q124 |
| 4-2749 | H | H | Et | A20 | J2 | Q124 |
| 4-2750 | H | H | Pr | A20 | J2 | Q124 |
| 4-2751 | H | H | cPr | A20 | J2 | Q124 |
| 4-2752 | H | H | ethynyl | A20 | J2 | Q124 |
| 4-2753 | F | H | Me | A20 | J2 | Q124 |
| 4-2754 | F | F | Me | A20 | J2 | Q124 |
| 4-2755 | Me | H | Me | A20 | J2 | Q124 |
| 4-2756 | Me | Me | Me | A20 | J2 | Q124 |
| 4-2757 | H | H | H | A21 | J2 | Q124 |
| 4-2758 | H | H | Cl | A21 | J2 | Q124 |
| 4-2759 | H | H | Br | A21 | J2 | Q124 |
| 4-2760 | H | H | CH₂F | A21 | J2 | Q124 |
| 4-2761 | H | H | CHF2 | A21 | J2 | Q124 |
| 4-2762 | H | H | Et | A21 | J2 | Q124 |
| 4-2763 | H | H | Pr | A21 | J2 | Q124 |
| 4-2764 | H | H | cPr | A21 | J2 | Q124 |
| 4-2765 | H | H | ethynyl | A21 | J2 | Q124 |
| 4-2766 | F | H | Me | A21 | J2 | Q124 |
| 4-2767 | F | F | Me | A21 | J2 | Q124 |
| 4-2768 | Me | H | Me | A21 | J2 | Q124 |
| 4-2769 | Me | Me | Me | A21 | J2 | Q124 |
| 4-2770 | H | H | H | A22 | J2 | Q124 |
| 4-2771 | H | H | Cl | A22 | J2 | Q124 |
| 4-2772 | H | H | Br | A22 | J2 | Q124 |
| 4-2773 | H | H | CH₂F | A22 | J2 | Q124 |
| 4-2774 | H | H | CHF2 | A22 | J2 | Q124 |
| 4-2775 | H | H | Et | A22 | J2 | Q124 |
| 4-2776 | H | H | Pr | A22 | J2 | Q124 |
| 4-2777 | H | H | cPr | A22 | J2 | Q124 |
| 4-2778 | H | H | ethynyl | A22 | J2 | Q124 |
| 4-2779 | F | H | Me | A22 | J2 | Q124 |
| 4-2780 | F | F | Me | A22 | J2 | Q124 |
| 4-2781 | Me | H | Me | A22 | J2 | Q124 |
| 4-2782 | Me | Me | Me | A22 | J2 | Q124 |
| 4-2783 | H | H | H | A26 | J2 | Q124 |
| 4-2784 | H | H | Cl | A26 | J2 | Q124 |
| 4-2785 | H | H | Br | A26 | J2 | Q124 |
| 4-2786 | H | H | CH₂F | A26 | J2 | Q124 |
| 4-2787 | H | H | CHF2 | A26 | J2 | Q124 |
| 4-2788 | H | H | Et | A26 | J2 | Q124 |
| 4-2789 | H | H | Pr | A26 | J2 | Q124 |
| 4-2790 | H | H | cPr | A26 | J2 | Q124 |
| 4-2791 | H | H | ethynyl | A26 | J2 | Q124 |
| 4-2792 | F | H | Me | A26 | J2 | Q124 |
| 4-2793 | F | F | Me | A26 | J2 | Q124 |
| 4-2794 | Me | H | Me | A26 | J2 | Q124 |
| 4-2795 | Me | Me | Me | A26 | J2 | Q124 |
| 4-2796 | H | H | H | A27 | J2 | Q124 |
| 4-2797 | H | H | Cl | A27 | J2 | Q124 |
| 4-2798 | H | H | Br | A27 | J2 | Q124 |
| 4-2799 | H | H | CH₂F | A27 | J2 | Q124 |
| 4-2800 | H | H | CHF2 | A27 | J2 | Q124 |
| 4-2801 | H | H | Et | A27 | J2 | Q124 |
| 4-2802 | H | H | Pr | A27 | J2 | Q124 |
| 4-2803 | H | H | cPr | A27 | J2 | Q124 |
| 4-2804 | H | H | ethynyl | A27 | J2 | Q124 |
| 4-2805 | F | H | Me | A27 | J2 | Q124 |
| 4-2806 | F | F | Me | A27 | J2 | Q124 |
| 4-2807 | Me | H | Me | A27 | J2 | Q124 |
| 4-2808 | Me | Me | Me | A27 | J2 | Q124 |
| 4-2809 | H | H | H | A28 | J2 | Q124 |
| 4-2810 | H | H | Cl | A28 | J2 | Q124 |
| 4-2811 | H | H | Br | A28 | J2 | Q124 |
| 4-2812 | H | H | CH₂F | A28 | J2 | Q124 |
| 4-2813 | H | H | CHF2 | A28 | J2 | Q124 |
| 4-2814 | H | H | Et | A28 | J2 | Q124 |
| 4-2815 | H | H | Pr | A28 | J2 | Q124 |
| 4-2816 | H | H | cPr | A28 | J2 | Q124 |
| 4-2817 | H | H | ethynyl | A28 | J2 | Q124 |
| 4-2818 | F | H | Me | A28 | J2 | Q124 |
| 4-2819 | F | F | Me | A28 | J2 | Q124 |
| 4-2820 | Me | H | Me | A28 | J2 | Q124 |
| 4-2821 | Me | Me | Me | A28 | J2 | Q124 |
| 4-2822 | H | H | H | A29 | J2 | Q124 |
| 4-2823 | H | H | Cl | A29 | J2 | Q124 |
| 4-2824 | H | H | Br | A29 | J2 | Q124 |
| 4-2825 | H | H | CH₂F | A29 | J2 | Q124 |
| 4-2826 | H | H | CHF2 | A29 | J2 | Q124 |
| 4-2827 | H | H | Et | A29 | J2 | Q124 |
| 4-2828 | H | H | Pr | A29 | J2 | Q124 |
| 4-2829 | H | H | cPr | A29 | J2 | Q124 |
| 4-2830 | H | H | ethynyl | A29 | J2 | Q124 |
| 4-2831 | F | H | Me | A29 | J2 | Q124 |
| 4-2832 | F | F | Me | A29 | J2 | Q124 |
| 4-2833 | Me | H | Me | A29 | J2 | Q124 |
| 4-2834 | Me | Me | Me | A29 | J2 | Q124 |
| 4-2835 | H | H | H | A30 | J2 | Q124 |
| 4-2836 | H | H | Cl | A30 | J2 | Q124 |
| 4-2837 | H | H | Br | A30 | J2 | Q124 |
| 4-2838 | H | H | CH₂F | A30 | J2 | Q124 |
| 4-2839 | H | H | CHF2 | A30 | J2 | Q124 |
| 4-2840 | H | H | Et | A30 | J2 | Q124 |
| 4-2841 | H | H | Pr | A30 | J2 | Q124 |
| 4-2842 | H | H | cPr | A30 | J2 | Q124 |
| 4-2843 | H | H | ethynyl | A30 | J2 | Q124 |
| 4-2844 | F | H | Me | A30 | J2 | Q124 |
| 4-2845 | F | F | Me | A30 | J2 | Q124 |
| 4-2846 | Me | H | Me | A30 | J2 | Q124 |
| 4-2847 | Me | Me | Me | A30 | J2 | Q124 |
| 4-2848 | H | H | H | A31 | J2 | Q124 |
| 4-2849 | H | H | Cl | A31 | J2 | Q124 |
| 4-2850 | H | H | Br | A31 | J2 | Q124 |
| 4-2851 | H | H | CH₂F | A31 | J2 | Q124 |
| 4-2852 | H | H | CHF2 | A31 | J2 | Q124 |
| 4-2853 | H | H | Et | A31 | J2 | Q124 |
| 4-2854 | H | H | Pr | A31 | J2 | Q124 |
| 4-2855 | H | H | cPr | A31 | J2 | Q124 |
| 4-2856 | H | H | ethynyl | A31 | J2 | Q124 |
| 4-2857 | F | H | Me | A31 | J2 | Q124 |
| 4-2858 | F | F | Me | A31 | J2 | Q124 |
| 4-2859 | Me | H | Me | A31 | J2 | Q124 |
| 4-2860 | Me | Me | Me | A31 | J2 | Q124 |
| 4-2861 | H | H | H | A51 | J2 | Q124 |
| 4-2862 | H | H | Cl | A51 | J2 | Q124 |
| 4-2863 | H | H | Br | A51 | J2 | Q124 |
| 4-2864 | H | H | CH₂F | A51 | J2 | Q124 |
| 4-2865 | H | H | CHF2 | A51 | J2 | Q124 |
| 4-2866 | H | H | Et | A51 | J2 | Q124 |
| 4-2867 | H | H | Pr | A51 | J2 | Q124 |
| 4-2868 | H | H | cPr | A51 | J2 | Q124 |
| 4-2869 | H | H | ethynyl | A51 | J2 | Q124 |
| 4-2870 | F | H | Me | A51 | J2 | Q124 |
| 4-2871 | F | F | Me | A51 | J2 | Q124 |
| 4-2872 | Me | H | Me | A51 | J2 | Q124 |
| 4-2873 | Me | Me | Me | A51 | J2 | Q124 |
| 4-2874 | H | H | H | A1 | J1 | Q124 |
| 4-2875 | H | H | Cl | A1 | J1 | Q124 |
| 4-2876 | H | H | Br | A1 | J1 | Q124 |
| 4-2877 | H | H | CH₂F | A1 | J1 | Q124 |
| 4-2878 | H | H | CHF2 | A1 | J1 | Q124 |
| 4-2879 | H | H | Et | A1 | J1 | Q124 |
| 4-2880 | H | H | Pr | A1 | J1 | Q124 |
| 4-2881 | H | H | cPr | A1 | J1 | Q124 |
| 4-2882 | H | H | ethynyl | A1 | J1 | Q124 |
| 4-2883 | F | H | Me | A1 | J1 | Q124 |
| 4-2884 | F | F | Me | A1 | J1 | Q124 |
| 4-2885 | Me | H | Me | A1 | J1 | Q124 |
| 4-2886 | Me | Me | Me | A1 | J1 | Q124 |
| 4-2887 | H | H | H | A2 | J1 | Q124 |
| 4-2888 | H | H | Cl | A2 | J1 | Q124 |
| 4-2889 | H | H | Br | A2 | J1 | Q124 |
| 4-2890 | H | H | CH₂F | A2 | J1 | Q124 |
| 4-2891 | H | H | CHF2 | A2 | J1 | Q124 |
| 4-2892 | H | H | Et | A2 | J1 | Q124 |
| 4-2893 | H | H | Pr | A2 | J1 | Q124 |
| 4-2894 | H | H | cPr | A2 | J1 | Q124 |
| 4-2895 | H | H | ethynyl | A2 | J1 | Q124 |
| 4-2896 | F | H | Me | A2 | J1 | Q124 |
| 4-2897 | F | F | Me | A2 | J1 | Q124 |
| 4-2898 | Me | H | Me | A2 | J1 | Q124 |
| 4-2899 | Me | Me | Me | A2 | J1 | Q124 |
| 4-2900 | H | H | H | A6 | J1 | Q124 |
| 4-2901 | H | H | Cl | A6 | J1 | Q124 |
| 4-2902 | H | H | Br | A6 | J1 | Q124 |
| 4-2903 | H | H | CH₂F | A6 | J1 | Q124 |
| 4-2904 | H | H | CHF2 | A6 | J1 | Q124 |
| 4-2905 | H | H | Et | A6 | J1 | Q124 |
| 4-2906 | H | H | Pr | A6 | J1 | Q124 |
| 4-2907 | H | H | cPr | A6 | J1 | Q124 |
| 4-2908 | H | H | ethynyl | A6 | J1 | Q124 |
| 4-2909 | F | H | Me | A6 | J1 | Q124 |
| 4-2910 | F | F | Me | A6 | J1 | Q124 |
| 4-2911 | Me | H | Me | A6 | J1 | Q124 |
| 4-2912 | Me | Me | Me | A6 | J1 | Q124 |
| 4-2913 | H | H | H | A9 | J1 | Q124 |
| 4-2914 | H | H | Cl | A9 | J1 | Q124 |
| 4-2915 | H | H | Br | A9 | J1 | Q124 |
| 4-2916 | H | H | CH₂F | A9 | J1 | Q124 |
| 4-2917 | H | H | CHF2 | A9 | J1 | Q124 |
| 4-2918 | H | H | Et | A9 | J1 | Q124 |
| 4-2919 | H | H | Pr | A9 | J1 | Q124 |
| 4-2920 | H | H | cPr | A9 | J1 | Q124 |
| 4-2921 | H | H | ethynyl | A9 | J1 | Q124 |
| 4-2922 | F | H | Me | A9 | J1 | Q124 |
| 4-2923 | F | F | Me | A9 | J1 | Q124 |
| 4-2924 | Me | H | Me | A9 | J1 | Q124 |
| 4-2925 | Me | Me | Me | A9 | J1 | Q124 |
| 4-2926 | H | H | H | A11 | J1 | Q124 |
| 4-2927 | H | H | Cl | A11 | J1 | Q124 |
| 4-2928 | H | H | Br | A11 | J1 | Q124 |
| 4-2929 | H | H | CH₂F | A11 | J1 | Q124 |
| 4-2930 | H | H | CHF2 | A11 | J1 | Q124 |
| 4-2931 | H | H | Et | A11 | J1 | Q124 |
| 4-2932 | H | H | Pr | A11 | J1 | Q124 |
| 4-2933 | H | H | cPr | A11 | J1 | Q124 |
| 4-2934 | H | H | ethynyl | A11 | J1 | Q124 |
| 4-2935 | F | H | Me | A11 | J1 | Q124 |
| 4-2936 | F | F | Me | A11 | J1 | Q124 |
| 4-2937 | Me | H | Me | A11 | J1 | Q124 |
| 4-2938 | Me | Me | Me | A11 | J1 | Q124 |
| 4-2939 | H | H | H | A12 | J1 | Q124 |
| 4-2940 | H | H | Cl | A12 | J1 | Q124 |
| 4-2941 | H | H | Br | A12 | J1 | Q124 |
| 4-2942 | H | H | CH₂F | A12 | J1 | Q124 |
| 4-2943 | H | H | CHF2 | A12 | J1 | Q124 |
| 4-2944 | H | H | Et | A12 | J1 | Q124 |
| 4-2945 | H | H | Pr | A12 | J1 | Q124 |
| 4-2946 | H | H | cPr | A12 | J1 | Q124 |
| 4-2947 | H | H | ethynyl | A12 | J1 | Q124 |
| 4-2948 | F | H | Me | A12 | J1 | Q124 |
| 4-2949 | F | F | Me | A12 | J1 | Q124 |
| 4-2950 | Me | H | Me | A12 | J1 | Q124 |
| 4-2951 | Me | Me | Me | A12 | J1 | Q124 |
| 4-2952 | H | H | H | A19 | J1 | Q124 |
| 4-2953 | H | H | Cl | A19 | J1 | Q124 |
| 4-2954 | H | H | Br | A19 | J1 | Q124 |
| 4-2955 | H | H | CH₂F | A19 | J1 | Q124 |
| 4-2956 | H | H | CHF2 | A19 | J1 | Q124 |
| 4-2957 | H | H | Et | A19 | J1 | Q124 |
| 4-2958 | H | H | Pr | A19 | J1 | Q124 |
| 4-2959 | H | H | cPr | A19 | J1 | Q124 |
| 4-2960 | H | H | ethynyl | A19 | J1 | Q124 |
| 4-2961 | F | H | Me | A19 | J1 | Q124 |
| 4-2962 | F | F | Me | A19 | J1 | Q124 |
| 4-2963 | Me | H | Me | A19 | J1 | Q124 |
| 4-2964 | Me | Me | Me | A19 | J1 | Q124 |
| 4-2965 | H | H | H | A20 | J1 | Q124 |
| 4-2966 | H | H | Cl | A20 | J1 | Q124 |
| 4-2967 | H | H | Br | A20 | J1 | Q124 |
| 4-2968 | H | H | CH₂F | A20 | J1 | Q124 |
| 4-2969 | H | H | CHF2 | A20 | J1 | Q124 |
| 4-2970 | H | H | Et | A20 | J1 | Q124 |
| 4-2971 | H | H | Pr | A20 | J1 | Q124 |
| 4-2972 | H | H | cPr | A20 | J1 | Q124 |
| 4-2973 | H | H | ethynyl | A20 | J1 | Q124 |
| 4-2974 | F | H | Me | A20 | J1 | Q124 |
| 4-2975 | F | F | Me | A20 | J1 | Q124 |
| 4-2976 | Me | H | Me | A20 | J1 | Q124 |
| 4-2977 | Me | Me | Me | A20 | J1 | Q124 |
| 4-2978 | H | H | H | A21 | J1 | Q124 |
| 4-2979 | H | H | Cl | A21 | J1 | Q124 |
| 4-2980 | H | H | Br | A21 | J1 | Q124 |
| 4-2981 | H | H | CH₂F | A21 | J1 | Q124 |
| 4-2982 | H | H | CHF2 | A21 | J1 | Q124 |
| 4-2983 | H | H | Et | A21 | J1 | Q124 |
| 4-2984 | H | H | Pr | A21 | J1 | Q124 |
| 4-2985 | H | H | cPr | A21 | J1 | Q124 |
| 4-2986 | H | H | ethynyl | A21 | J1 | Q124 |
| 4-2987 | F | H | Me | A21 | J1 | Q124 |
| 4-2988 | F | F | Me | A21 | J1 | Q124 |
| 4-2989 | Me | H | Me | A21 | J1 | Q124 |
| 4-2990 | Me | Me | Me | A21 | J1 | Q124 |
| 4-2991 | H | H | H | A22 | J1 | Q124 |
| 4-2992 | H | H | Cl | A22 | J1 | Q124 |
| 4-2993 | H | H | Br | A22 | J1 | Q124 |
| 4-2994 | H | H | CH₂F | A22 | J1 | Q124 |
| 4-2995 | H | H | CHF2 | A22 | J1 | Q124 |
| 4-2996 | H | H | Et | A22 | J1 | Q124 |
| 4-2997 | H | H | Pr | A22 | J1 | Q124 |
| 4-2998 | H | H | cPr | A22 | J1 | Q124 |
| 4-2999 | H | H | ethynyl | A22 | J1 | Q124 |
| 4-3000 | F | H | Me | A22 | J1 | Q124 |
| 4-3001 | F | F | Me | A22 | J1 | Q124 |
| 4-3002 | Me | H | Me | A22 | J1 | Q124 |
| 4-3003 | Me | Me | Me | A22 | J1 | Q124 |
| 4-3004 | H | H | H | A26 | J1 | Q124 |
| 4-3005 | H | H | Cl | A26 | J1 | Q124 |
| 4-3006 | H | H | Br | A26 | J1 | Q124 |
| 4-3007 | H | H | CH₂F | A26 | J1 | Q124 |
| 4-3008 | H | H | CHF2 | A26 | J1 | Q124 |
| 4-3009 | H | H | Et | A26 | J1 | Q124 |
| 4-3010 | H | H | Pr | A26 | J1 | Q124 |
| 4-3011 | H | H | cPr | A26 | J1 | Q124 |
| 4-3012 | H | H | ethynyl | A26 | J1 | Q124 |
| 4-3013 | F | H | Me | A26 | J1 | Q124 |
| 4-3014 | F | F | Me | A26 | J1 | Q124 |
| 4-3015 | Me | H | Me | A26 | J1 | Q124 |
| 4-3016 | Me | Me | Me | A26 | J1 | Q124 |
| 4-3017 | H | H | H | A27 | J1 | Q124 |
| 4-3018 | H | H | Cl | A27 | J1 | Q124 |
| 4-3019 | H | H | Br | A27 | J1 | Q124 |
| 4-3020 | H | H | CH₂F | A27 | J1 | Q124 |
| 4-3021 | H | H | CHF2 | A27 | J1 | Q124 |
| 4-3022 | H | H | Et | A27 | J1 | Q124 |
| 4-3023 | H | H | Pr | A27 | J1 | Q124 |
| 4-3024 | H | H | cPr | A27 | J1 | Q124 |
| 4-3025 | H | H | ethynyl | A27 | J1 | Q124 |
| 4-3026 | F | H | Me | A27 | J1 | Q124 |
| 4-3027 | F | F | Me | A27 | J1 | Q124 |
| 4-3028 | Me | H | Me | A27 | J1 | Q124 |
| 4-3029 | Me | Me | Me | A27 | J1 | Q124 |
| 4-3030 | H | H | H | A28 | J1 | Q124 |
| 4-3031 | H | H | Cl | A28 | J1 | Q124 |
| 4-3032 | H | H | Br | A28 | J1 | Q124 |
| 4-3033 | H | H | CH₂F | A28 | J1 | Q124 |
| 4-3034 | H | H | CHF2 | A28 | J1 | Q124 |
| 4-3035 | H | H | Et | A28 | J1 | Q124 |
| 4-3036 | H | H | Pr | A28 | J1 | Q124 |
| 4-3037 | H | H | cPr | A28 | J1 | Q124 |
| 4-3038 | H | H | ethynyl | A28 | J1 | Q124 |
| 4-3039 | F | H | Me | A28 | J1 | Q124 |
| 4-3040 | F | F | Me | A28 | J1 | Q124 |
| 4-3041 | Me | H | Me | A28 | J1 | Q124 |
| 4-3042 | Me | Me | Me | A28 | J1 | Q124 |
| 4-3043 | H | H | H | A29 | J1 | Q124 |
| 4-3044 | H | H | Cl | A29 | J1 | Q124 |
| 4-3045 | H | H | Br | A29 | J1 | Q124 |
| 4-3046 | H | H | CH₂F | A29 | J1 | Q124 |
| 4-3047 | H | H | CHF2 | A29 | J1 | Q124 |
| 4-3048 | H | H | Et | A29 | J1 | Q124 |
| 4-3049 | H | H | Pr | A29 | J1 | Q124 |
| 4-3050 | H | H | cPr | A29 | J1 | Q124 |
| 4-3051 | H | H | ethynyl | A29 | J1 | Q124 |
| 4-3052 | F | H | Me | A29 | J1 | Q124 |
| 4-3053 | F | F | Me | A29 | J1 | Q124 |
| 4-3054 | Me | H | Me | A29 | J1 | Q124 |
| 4-3055 | Me | Me | Me | A29 | J1 | Q124 |
| 4-3056 | H | H | H | A30 | J1 | Q124 |
| 4-3057 | H | H | Cl | A30 | J1 | Q124 |
| 4-3058 | H | H | Br | A30 | J1 | Q124 |
| 4-3059 | H | H | CH₂F | A30 | J1 | Q124 |
| 4-3060 | H | H | CHF2 | A30 | J1 | Q124 |
| 4-3061 | H | H | Et | A30 | J1 | Q124 |
| 4-3062 | H | H | Pr | A30 | J1 | Q124 |
| 4-3063 | H | H | cPr | A30 | J1 | Q124 |
| 4-3064 | H | H | ethynyl | A30 | J1 | Q124 |
| 4-3065 | F | H | Me | A30 | J1 | Q124 |
| 4-3066 | F | F | Me | A30 | J1 | Q124 |
| 4-3067 | Me | H | Me | A30 | J1 | Q124 |
| 4-3068 | Me | Me | Me | A30 | J1 | Q124 |
| 4-3069 | H | H | H | A31 | J1 | Q124 |
| 4-3070 | H | H | Cl | A31 | J1 | Q124 |
| 4-3071 | H | H | Br | A31 | J1 | Q124 |
| 4-3072 | H | H | CH₂F | A31 | J1 | Q124 |
| 4-3073 | H | H | CHF2 | A31 | J1 | Q124 |
| 4-3074 | H | H | Et | A31 | J1 | Q124 |
| 4-3075 | H | H | Pr | A31 | J1 | Q124 |
| 4-3076 | H | H | cPr | A31 | J1 | Q124 |
| 4-3077 | H | H | ethynyl | A31 | J1 | Q124 |
| 4-3078 | F | H | Me | A31 | J1 | Q124 |
| 4-3079 | F | F | Me | A31 | J1 | Q124 |
| 4-3080 | Me | H | Me | A31 | J1 | Q124 |
| 4-3081 | Me | Me | Me | A31 | J1 | Q124 |
| 4-3082 | H | H | H | A51 | J1 | Q124 |
| 4-3083 | H | H | Cl | A51 | J1 | Q124 |
| 4-3084 | H | H | Br | A51 | J1 | Q124 |
| 4-3085 | H | H | CH₂F | A51 | J1 | Q124 |
| 4-3086 | H | H | CHF2 | A51 | J1 | Q124 |
| 4-3087 | H | H | Et | A51 | J1 | Q124 |
| 4-3088 | H | H | Pr | A51 | J1 | Q124 |
| 4-3089 | H | H | cPr | A51 | J1 | Q124 |
| 4-3090 | H | H | ethynyl | A51 | J1 | Q124 |
| 4-3091 | F | H | Me | A51 | J1 | Q124 |
| 4-3092 | F | F | Me | A51 | J1 | Q124 |
| 4-3093 | Me | H | Me | A51 | J1 | Q124 |
| 4-3094 | Me | Me | Me | A51 | J1 | Q124 |
| 4-3095 | H | H | H | A1 | J2 | Q125 |
| 4-3096 | H | H | Cl | A1 | J2 | Q125 |
| 4-3097 | H | H | Br | A1 | J2 | Q125 |
| 4-3098 | H | H | CH₂F | A1 | J2 | Q125 |
| 4-3099 | H | H | CHF2 | A1 | J2 | Q125 |
| 4-3100 | H | H | Et | A1 | J2 | Q125 |
| 4-3101 | H | H | Pr | A1 | J2 | Q125 |
| 4-3102 | H | H | cPr | A1 | J2 | Q125 |
| 4-3103 | H | H | ethynyl | A1 | J2 | Q125 |
| 4-3104 | F | H | Me | A1 | J2 | Q125 |
| 4-3105 | F | F | Me | A1 | J2 | Q125 |
| 4-3106 | Me | H | Me | A1 | J2 | Q125 |
| 4-3107 | Me | Me | Me | A1 | J2 | Q125 |
| 4-3108 | H | H | H | A2 | J2 | Q125 |
| 4-3109 | H | H | Cl | A2 | J2 | Q125 |
| 4-3110 | H | H | Br | A2 | J2 | Q125 |
| 4-3111 | H | H | CH₂F | A2 | J2 | Q125 |
| 4-3112 | H | H | CHF2 | A2 | J2 | Q125 |
| 4-3113 | H | H | Et | A2 | J2 | Q125 |
| 4-3114 | H | H | Pr | A2 | J2 | Q125 |
| 4-3115 | H | H | cPr | A2 | J2 | Q125 |
| 4-3116 | H | H | ethynyl | A2 | J2 | Q125 |
| 4-3117 | F | H | Me | A2 | J2 | Q125 |
| 4-3118 | F | F | Me | A2 | J2 | Q125 |
| 4-3119 | Me | H | Me | A2 | J2 | Q125 |
| 4-3120 | Me | Me | Me | A2 | J2 | Q125 |
| 4-3121 | H | H | H | A6 | J2 | Q125 |
| 4-3122 | H | H | Cl | A6 | J2 | Q125 |
| 4-3123 | H | H | Br | A6 | J2 | Q125 |
| 4-3124 | H | H | CH₂F | A6 | J2 | Q125 |
| 4-3125 | H | H | CHF2 | A6 | J2 | Q125 |
| 4-3126 | H | H | Et | A6 | J2 | Q125 |
| 4-3127 | H | H | Pr | A6 | J2 | Q125 |
| 4-3128 | H | H | cPr | A6 | J2 | Q125 |
| 4-3129 | H | H | ethynyl | A6 | J2 | Q125 |
| 4-3130 | F | H | Me | A6 | J2 | Q125 |
| 4-3131 | F | F | Me | A6 | J2 | Q125 |
| 4-3132 | Me | H | Me | A6 | J2 | Q125 |
| 4-3133 | Me | Me | Me | A6 | J2 | Q125 |
| 4-3134 | H | H | H | A9 | J2 | Q125 |
| 4-3135 | H | H | Cl | A9 | J2 | Q125 |
| 4-3136 | H | H | Br | A9 | J2 | Q125 |
| 4-3137 | H | H | CH₂F | A9 | J2 | Q125 |
| 4-3138 | H | H | CHF2 | A9 | J2 | Q125 |
| 4-3139 | H | H | Et | A9 | J2 | Q125 |
| 4-3140 | H | H | Pr | A9 | J2 | Q125 |
| 4-3141 | H | H | cPr | A9 | J2 | Q125 |
| 4-3142 | H | H | ethynyl | A9 | J2 | Q125 |
| 4-3143 | F | H | Me | A9 | J2 | Q125 |
| 4-3144 | F | F | Me | A9 | J2 | Q125 |
| 4-3145 | Me | H | Me | A9 | J2 | Q125 |
| 4-3146 | Me | Me | Me | A9 | J2 | Q125 |
| 4-3147 | H | H | H | A11 | J2 | Q125 |
| 4-3148 | H | H | Cl | A11 | J2 | Q125 |
| 4-3149 | H | H | Br | A11 | J2 | Q125 |
| 4-3150 | H | H | CH₂F | A11 | J2 | Q125 |
| 4-3151 | H | H | CHF2 | A11 | J2 | Q125 |
| 4-3152 | H | H | Et | A11 | J2 | Q125 |
| 4-3153 | H | H | Pr | A11 | J2 | Q125 |
| 4-3154 | H | H | cPr | A11 | J2 | Q125 |
| 4-3155 | H | H | ethynyl | A11 | J2 | Q125 |
| 4-3156 | F | H | Me | A11 | J2 | Q125 |
| 4-3157 | F | F | Me | A11 | J2 | Q125 |
| 4-3158 | Me | H | Me | A11 | J2 | Q125 |
| 4-3159 | Me | Me | Me | A11 | J2 | Q125 |
| 4-3160 | H | H | H | A12 | J2 | Q125 |
| 4-3161 | H | H | Cl | A12 | J2 | Q125 |
| 4-3162 | H | H | Br | A12 | J2 | Q125 |
| 4-3163 | H | H | CH₂F | A12 | J2 | Q125 |
| 4-3164 | H | H | CHF2 | A12 | J2 | Q125 |
| 4-3165 | H | H | Et | A12 | J2 | Q125 |
| 4-3166 | H | H | Pr | A12 | J2 | Q125 |
| 4-3167 | H | H | cPr | A12 | J2 | Q125 |
| 4-3168 | H | H | ethynyl | A12 | J2 | Q125 |
| 4-3169 | F | H | Me | A12 | J2 | Q125 |
| 4-3170 | F | F | Me | A1 | J2 | Q125 |
| 4-3171 | Me | H | Me | A12 | J2 | Q125 |
| 4-3172 | Me | Me | Me | A12 | J2 | Q125 |
| 4-3173 | H | H | H | A19 | J2 | Q125 |
| 4-3174 | H | H | Cl | A19 | J2 | Q125 |
| 4-3175 | H | H | Br | A19 | J2 | Q125 |
| 4-3176 | H | H | CH₂F | A19 | J2 | Q125 |
| 4-3177 | H | H | CHF2 | A19 | J2 | Q125 |
| 4-3178 | H | H | Et | A19 | J2 | Q125 |
| 4-3179 | H | H | Pr | A19 | J2 | Q125 |
| 4-3180 | H | H | cPr | A19 | J2 | Q125 |
| 4-3181 | H | H | ethynyl | A19 | J2 | Q125 |
| 4-3182 | F | H | Me | A19 | J2 | Q125 |
| 4-3183 | F | F | Me | A19 | J2 | Q125 |
| 4-3184 | Me | H | Me | A19 | J2 | Q125 |
| 4-3185 | Me | Me | Me | A19 | J2 | Q125 |
| 4-3186 | H | H | H | A20 | J2 | Q125 |
| 4-3187 | H | H | Cl | A20 | J2 | Q125 |
| 4-3188 | H | H | Br | A20 | J2 | Q125 |
| 4-3189 | H | H | CH₂F | A20 | J2 | Q125 |
| 4-3190 | H | H | CHF2 | A20 | J2 | Q125 |
| 4-3191 | H | H | Et | A20 | J2 | Q125 |
| 4-3192 | H | H | Pr | A20 | J2 | Q125 |
| 4-3193 | H | H | cPr | A20 | J2 | Q125 |
| 4-3194 | H | H | ethynyl | A20 | J2 | Q125 |
| 4-3195 | F | H | Me | A20 | J2 | Q125 |
| 4-3196 | F | F | Me | A20 | J2 | Q125 |
| 4-3197 | Me | H | Me | A20 | J2 | Q125 |
| 4-3198 | Me | Me | Me | A20 | J2 | Q125 |
| 4-3199 | H | H | H | A21 | J2 | Q125 |
| 4-3200 | H | H | Cl | A21 | J2 | Q125 |
| 4-3201 | H | H | Br | A21 | J2 | Q125 |
| 4-3202 | H | H | CH₂F | A21 | J2 | Q125 |
| 4-3203 | H | H | CHF2 | A21 | J2 | Q125 |
| 4-3204 | H | H | Et | A21 | J2 | Q125 |
| 4-3205 | H | H | Pr | A21 | J2 | Q125 |
| 4-3206 | H | H | cPr | A21 | J2 | Q125 |
| 4-3207 | H | H | ethynyl | A21 | J2 | Q125 |
| 4-3208 | F | H | Me | A21 | J2 | Q125 |
| 4-3209 | F | F | Me | A21 | J2 | Q125 |
| 4-3210 | Me | H | Me | A21 | J2 | Q125 |
| 4-3211 | Me | Me | Me | A21 | J2 | Q125 |
| 4-3212 | H | H | H | A22 | J2 | Q125 |
| 4-3213 | H | H | Cl | A22 | J2 | Q125 |
| 4-3214 | H | H | Br | A22 | J2 | Q125 |
| 4-3215 | H | H | CH₂F | A22 | J2 | Q125 |
| 4-3216 | H | H | CHF2 | A22 | J2 | Q125 |
| 4-3217 | H | H | Et | A22 | J2 | Q125 |
| 4-3218 | H | H | Pr | A22 | J2 | Q125 |
| 4-3219 | H | H | cPr | A22 | J2 | Q125 |
| 4-3220 | H | H | ethynyl | A22 | J2 | Q125 |
| 4-3221 | F | H | Me | A22 | J2 | Q125 |
| 4-3222 | F | F | Me | A22 | J2 | Q125 |
| 4-3223 | Me | H | Me | A22 | J2 | Q125 |
| 4-3224 | Me | Me | Me | A22 | J2 | Q125 |
| 4-3225 | H | H | H | A26 | J2 | Q125 |
| 4-3226 | H | H | Cl | A26 | J2 | Q125 |
| 4-3227 | H | H | Br | A26 | J2 | Q125 |
| 4-3228 | H | H | CH₂F | A26 | J2 | Q125 |
| 4-3229 | H | H | CHF2 | A26 | J2 | Q125 |
| 4-3230 | H | H | Et | A26 | J2 | Q125 |
| 4-3231 | H | H | Pr | A26 | J2 | Q125 |
| 4-3232 | H | H | cPr | A26 | J2 | Q125 |
| 4-3233 | H | H | ethynyl | A26 | J2 | Q125 |
| 4-3234 | F | H | Me | A26 | J2 | Q125 |
| 4-3235 | F | F | Me | A26 | J2 | Q125 |
| 4-3236 | Me | H | Me | A26 | J2 | Q125 |
| 4-3237 | Me | Me | Me | A26 | J2 | Q125 |
| 4-3238 | H | H | H | A27 | J2 | Q125 |
| 4-3239 | H | H | Cl | A27 | J2 | Q125 |
| 4-3240 | H | H | Br | A27 | J2 | Q125 |
| 4-3241 | H | H | CH₂F | A27 | J2 | Q125 |
| 4-3242 | H | H | CHF2 | A27 | J2 | Q125 |
| 4-3243 | H | H | Et | A27 | J2 | Q125 |
| 4-3244 | H | H | Pr | A27 | J2 | Q125 |
| 4-3245 | H | H | cPr | A27 | J2 | Q125 |
| 4-3246 | H | H | ethynyl | A27 | J2 | Q125 |
| 4-3247 | F | H | Me | A27 | J2 | Q125 |
| 4-3248 | F | F | Me | A27 | J2 | Q125 |
| 4-3249 | Me | H | Me | A27 | J2 | Q125 |
| 4-3250 | Me | Me | Me | A27 | J2 | Q125 |
| 4-3251 | H | H | H | A28 | J2 | Q125 |
| 4-3252 | H | H | Cl | A28 | J2 | Q125 |
| 4-3253 | H | H | Br | A28 | J2 | Q125 |
| 4-3254 | H | H | CH₂F | A28 | J2 | Q125 |
| 4-3255 | H | H | CHF2 | A28 | J2 | Q125 |
| 4-3256 | H | H | Et | A28 | J2 | Q125 |
| 4-3257 | H | H | Pr | A28 | J2 | Q125 |
| 4-3258 | H | H | cPr | A28 | J2 | Q125 |
| 4-3259 | H | H | ethynyl | A28 | J2 | Q125 |
| 4-3260 | F | H | Me | A28 | J2 | Q125 |
| 4-3261 | F | F | Me | A28 | J2 | Q125 |
| 4-3262 | Me | H | Me | A28 | J2 | Q125 |
| 4-3263 | Me | Me | Me | A28 | J2 | Q125 |
| 4-3264 | H | H | H | A29 | J2 | Q125 |
| 4-3265 | H | H | Cl | A29 | J2 | Q125 |
| 4-3266 | H | H | Br | A29 | J2 | Q125 |
| 4-3267 | H | H | CH₂F | A29 | J2 | Q125 |
| 4-3268 | H | H | CHF2 | A29 | J2 | Q125 |
| 4-3269 | H | H | Et | A29 | J2 | Q125 |
| 4-3270 | H | H | Pr | A29 | J2 | Q125 |
| 4-3271 | H | H | cPr | A29 | J2 | Q125 |
| 4-3272 | H | H | ethynyl | A29 | J2 | Q125 |
| 4-3273 | F | H | Me | A29 | J2 | Q125 |
| 4-3274 | F | F | Me | A29 | J2 | Q125 |
| 4-3275 | Me | H | Me | A29 | J2 | Q125 |
| 4-3276 | Me | Me | Me | A29 | J2 | Q125 |
| 4-3277 | H | H | H | A30 | J2 | Q125 |
| 4-3278 | H | H | Cl | A30 | J2 | Q125 |
| 4-3279 | H | H | Br | A30 | J2 | Q125 |
| 4-3280 | H | H | CH₂F | A30 | J2 | Q125 |
| 4-3281 | H | H | CHF2 | A30 | J2 | Q125 |
| 4-3282 | H | H | Et | A30 | J2 | Q125 |
| 4-3283 | H | H | Pr | A30 | J2 | Q125 |
| 4-3284 | H | H | cPr | A30 | J2 | Q125 |
| 4-3285 | H | H | ethynyl | A30 | J2 | Q125 |
| 4-3286 | F | H | Me | A30 | J2 | Q125 |
| 4-3287 | F | F | Me | A30 | J2 | Q125 |
| 4-3288 | Me | H | Me | A30 | J2 | Q125 |
| 4-3289 | Me | Me | Me | A30 | J2 | Q125 |
| 4-3290 | H | H | H | A31 | J2 | Q125 |
| 4-3291 | H | H | Cl | A31 | J2 | Q125 |
| 4-3292 | H | H | Br | A31 | J2 | Q125 |
| 4-3293 | H | H | CH₂F | A31 | J2 | Q125 |
| 4-3294 | H | H | CHF2 | A31 | J2 | Q125 |
| 4-3295 | H | H | Et | A31 | J2 | Q125 |
| 4-3296 | H | H | Pr | A31 | J2 | Q125 |
| 4-3297 | H | H | cPr | A31 | J2 | Q125 |
| 4-3298 | H | H | ethynyl | A31 | J2 | Q125 |
| 4-3299 | F | H | Me | A31 | J2 | Q125 |
| 4-3300 | F | F | Me | A31 | J2 | Q125 |
| 4-3301 | Me | H | Me | A31 | J2 | Q125 |
| 4-3302 | Me | Me | Me | A31 | J2 | Q125 |
| 4-3303 | H | H | H | A51 | J2 | Q125 |
| 4-3304 | H | H | Cl | A51 | J2 | Q125 |
| 4-3305 | H | H | Br | A51 | J2 | Q125 |
| 4-3306 | H | H | CH₂F | A51 | J2 | Q125 |
| 4-3307 | H | H | CHF2 | A51 | J2 | Q125 |
| 4-3308 | H | H | Et | A51 | J2 | Q125 |
| 4-3309 | H | H | Pr | A51 | J2 | Q125 |
| 4-3310 | H | H | cPr | A51 | J2 | Q125 |
| 4-3311 | H | H | ethynyl | A51 | J2 | Q125 |
| 4-3312 | F | H | Me | A51 | J2 | Q125 |
| 4-3313 | F | F | Me | A51 | J2 | Q125 |
| 4-3314 | Me | H | Me | A51 | J2 | Q125 |
| 4-3315 | Me | Me | Me | A51 | J2 | Q125 |
| 4-3316 | H | H | H | A1 | J1 | Q125 |
| 4-3317 | H | H | Cl | A1 | J1 | Q125 |
| 4-3318 | H | H | Br | A1 | J1 | Q125 |
| 4-3319 | H | H | CH₂F | A1 | J1 | Q125 |
| 4-3320 | H | H | CHF2 | A1 | J1 | Q125 |
| 4-3321 | H | H | Et | A1 | J1 | Q125 |
| 4-3322 | H | H | Pr | A1 | J1 | Q125 |
| 4-3323 | H | H | cPr | A1 | J1 | Q125 |
| 4-3324 | H | H | ethynyl | A1 | J1 | Q125 |
| 4-3325 | F | H | Me | A1 | J1 | Q125 |
| 4-3326 | F | F | Me | A1 | J1 | Q125 |
| 4-3327 | Me | H | Me | A1 | J1 | Q125 |
| 4-3328 | Me | Me | Me | A1 | J1 | Q125 |
| 4-3329 | H | H | H | A2 | J1 | Q125 |
| 4-3330 | H | H | Cl | A2 | J1 | Q125 |
| 4-3331 | H | H | Br | A2 | J1 | Q125 |
| 4-3332 | H | H | CH₂F | A2 | J1 | Q125 |
| 4-3333 | H | H | CHF2 | A2 | J1 | Q125 |
| 4-3334 | H | H | Et | A2 | J1 | Q125 |
| 4-3335 | H | H | Pr | A2 | J1 | Q125 |
| 4-3336 | H | H | cPr | A2 | J1 | Q125 |
| 4-3337 | H | H | ethynyl | A2 | J1 | Q125 |
| 4-3338 | F | H | Me | A2 | J1 | Q125 |
| 4-3339 | F | F | Me | A2 | J1 | Q125 |
| 4-3340 | Me | H | Me | A2 | J1 | Q125 |
| 4-3341 | Me | Me | Me | A2 | J1 | Q125 |
| 4-3342 | H | H | H | A6 | J1 | Q125 |
| 4-3343 | H | H | Cl | A6 | J1 | Q125 |
| 4-3344 | H | H | Br | A6 | J1 | Q125 |
| 4-3345 | H | H | CH₂F | A6 | J1 | Q125 |
| 4-3346 | H | H | CHF2 | A6 | J1 | Q125 |
| 4-3347 | H | H | Et | A6 | J1 | Q125 |
| 4-3348 | H | H | Pr | A6 | J1 | Q125 |
| 4-3349 | H | H | cPr | A6 | J1 | Q125 |
| 4-3350 | H | H | ethynyl | A6 | J1 | Q125 |
| 4-3351 | F | H | Me | A6 | J1 | Q125 |
| 4-3352 | F | F | Me | A6 | J1 | Q125 |
| 4-3353 | Me | H | Me | A6 | J1 | Q125 |
| 4-3354 | Me | Me | Me | A6 | J1 | Q125 |
| 4-3355 | H | H | H | A9 | J1 | Q125 |
| 4-3356 | H | H | Cl | A9 | J1 | Q125 |
| 4-3357 | H | H | Br | A9 | J1 | Q125 |
| 4-3358 | H | H | CH₂F | A9 | J1 | Q125 |
| 4-3359 | H | H | CHF2 | A9 | J1 | Q125 |
| 4-3360 | H | H | Et | A9 | J1 | Q125 |
| 4-3361 | H | H | Pr | A9 | J1 | Q125 |
| 4-3362 | H | H | cPr | A9 | J1 | Q125 |
| 4-3363 | H | H | ethynyl | A9 | J1 | Q125 |
| 4-3364 | F | H | Me | A9 | J1 | Q125 |
| 4-3365 | F | F | Me | A9 | J1 | Q125 |
| 4-3366 | Me | H | Me | A9 | J1 | Q125 |
| 4-3367 | Me | Me | Me | A9 | J1 | Q125 |
| 4-3368 | H | H | H | A11 | J1 | Q125 |
| 4-3369 | H | H | Cl | A11 | J1 | Q125 |
| 4-3370 | H | H | Br | A11 | J1 | Q125 |
| 4-3371 | H | H | CH₂F | A11 | J1 | Q125 |
| 4-3372 | H | H | CHF2 | A11 | J1 | Q125 |
| 4-3373 | H | H | Et | A11 | J1 | Q125 |
| 4-3374 | H | H | Pr | A11 | J1 | Q125 |
| 4-3375 | H | H | cPr | A11 | J1 | Q125 |
| 4-3376 | H | H | ethynyl | A11 | J1 | Q125 |
| 4-3377 | F | H | Me | A11 | J1 | Q125 |
| 4-3378 | F | F | Me | A11 | J1 | Q125 |
| 4-3379 | Me | H | Me | A11 | J1 | Q125 |
| 4-3380 | Me | Me | Me | A11 | J1 | Q125 |
| 4-3381 | H | H | H | A12 | J1 | Q125 |
| 4-3382 | H | H | Cl | A12 | J1 | Q125 |
| 4-3383 | H | H | Br | A12 | J1 | Q125 |
| 4-3384 | H | H | CH₂F | A12 | J1 | Q125 |
| 4-3385 | H | H | CHF2 | A12 | J1 | Q125 |
| 4-3386 | H | H | Et | A12 | J1 | Q125 |
| 4-3387 | H | H | Pr | A12 | J1 | Q125 |
| 4-3388 | H | H | cPr | A12 | J1 | Q125 |
| 4-3389 | H | H | ethynyl | A12 | J1 | Q125 |
| 4-3390 | F | H | Me | A12 | J1 | Q125 |
| 4-3391 | F | F | Me | A12 | J1 | Q125 |
| 4-3392 | Me | H | Me | A12 | J1 | Q125 |
| 4-3393 | Me | Me | Me | A12 | J1 | Q125 |
| 4-3394 | H | H | H | A19 | J1 | Q125 |
| 4-3395 | H | H | Cl | A19 | J1 | Q125 |
| 4-3396 | H | H | Br | A19 | J1 | Q125 |
| 4-3397 | H | H | CH₂F | A19 | J1 | Q125 |
| 4-3398 | H | H | CHF2 | A19 | J1 | Q125 |
| 4-3399 | H | H | Et | A19 | J1 | Q125 |
| 4-3400 | H | H | Pr | A19 | J1 | Q125 |
| 4-3401 | H | H | cPr | A19 | J1 | Q125 |
| 4-3402 | H | H | ethynyl | A19 | J1 | Q125 |
| 4-3403 | F | H | Me | A19 | J1 | Q125 |
| 4-3404 | F | F | Me | A19 | J1 | Q125 |
| 4-3405 | Me | H | Me | A19 | J1 | Q125 |
| 4-3406 | Me | Me | Me | A19 | J1 | Q125 |
| 4-3407 | H | H | H | A20 | J1 | Q125 |
| 4-3408 | H | H | Cl | A20 | J1 | Q125 |
| 4-3409 | H | H | Br | A20 | J1 | Q125 |
| 4-3410 | H | H | CH₂F | A20 | J1 | Q125 |
| 4-3411 | H | H | CHF2 | A20 | J1 | Q125 |
| 4-3412 | H | H | Et | A20 | J1 | Q125 |
| 4-3413 | H | H | Pr | A20 | J1 | Q125 |
| 4-3414 | H | H | cPr | A20 | J1 | Q125 |
| 4-3415 | H | H | ethynyl | A20 | J1 | Q125 |
| 4-3416 | F | H | Me | A20 | J1 | Q125 |
| 4-3417 | F | F | Me | A20 | J1 | Q125 |
| 4-3418 | Me | H | Me | A20 | J1 | Q125 |
| 4-3419 | Me | Me | Me | A20 | J1 | Q125 |
| 4-3420 | H | H | H | A21 | J1 | Q125 |
| 4-3421 | H | H | Cl | A21 | J1 | Q125 |
| 4-3422 | H | H | Br | A21 | J1 | Q125 |
| 4-3423 | H | H | CH₂F | A21 | J1 | Q125 |
| 4-3424 | H | H | CHF2 | A21 | J1 | Q125 |
| 4-3425 | H | H | Et | A21 | J1 | Q125 |
| 4-3426 | H | H | Pr | A21 | J1 | Q125 |
| 4-3427 | H | H | cPr | A21 | J1 | Q125 |
| 4-3428 | H | H | ethynyl | A21 | J1 | Q125 |
| 4-3429 | F | H | Me | A21 | J1 | Q125 |
| 4-3430 | F | F | Me | A21 | J1 | Q125 |
| 4-3431 | Me | H | Me | A21 | J1 | Q125 |
| 4-3432 | Me | Me | Me | A21 | J1 | Q125 |
| 4-3433 | H | H | H | A22 | J1 | Q125 |
| 4-3434 | H | H | Cl | A22 | J1 | Q125 |
| 4-3435 | H | H | Br | A22 | J1 | Q125 |
| 4-3436 | H | H | CH₂F | A22 | J1 | Q125 |
| 4-3437 | H | H | CHF2 | A22 | J1 | Q125 |
| 4-3438 | H | H | Et | A22 | J1 | Q125 |
| 4-3439 | H | H | Pr | A22 | J1 | Q125 |
| 4-3440 | H | H | cPr | A22 | J1 | Q125 |
| 4-3441 | H | H | ethynyl | A22 | J1 | Q125 |
| 4-3442 | F | H | Me | A22 | J1 | Q125 |
| 4-3443 | F | F | Me | A22 | J1 | Q125 |
| 4-3444 | Me | H | Me | A22 | J1 | Q125 |
| 4-3445 | Me | Me | Me | A22 | J1 | Q125 |
| 4-3446 | H | H | H | A26 | J1 | Q125 |
| 4-3447 | H | H | Cl | A26 | J1 | Q125 |
| 4-3448 | H | H | Br | A26 | J1 | Q125 |
| 4-3449 | H | H | CH₂F | A26 | J1 | Q125 |
| 4-3450 | H | H | CHF2 | A26 | J1 | Q125 |
| 4-3451 | H | H | Et | A26 | J1 | Q125 |
| 4-3452 | H | H | Pr | A26 | J1 | Q125 |
| 4-3453 | H | H | cPr | A26 | J1 | Q125 |
| 4-3454 | H | H | ethynyl | A26 | J1 | Q125 |
| 4-3455 | F | H | Me | A26 | J1 | Q125 |
| 4-3456 | F | F | Me | A26 | J1 | Q125 |
| 4-3457 | Me | H | Me | A26 | J1 | Q125 |
| 4-3458 | Me | Me | Me | A26 | J1 | Q125 |
| 4-3459 | H | H | H | A27 | J1 | Q125 |
| 4-3460 | H | H | Cl | A27 | J1 | Q125 |
| 4-3461 | H | H | Br | A27 | J1 | Q125 |
| 4-3462 | H | H | CH₂F | A27 | J1 | Q125 |
| 4-3463 | H | H | CHF2 | A27 | J1 | Q125 |
| 4-3464 | H | H | Et | A27 | J1 | Q125 |
| 4-3465 | H | H | Pr | A27 | J1 | Q125 |
| 4-3466 | H | H | cPr | A27 | J1 | Q125 |
| 4-3467 | H | H | ethynyl | A27 | J1 | Q125 |
| 4-3468 | F | H | Me | A27 | J1 | Q125 |
| 4-3469 | F | F | Me | A27 | J1 | Q125 |
| 4-3470 | Me | H | Me | A27 | J1 | Q125 |
| 4-3471 | Me | Me | Me | A27 | J1 | Q125 |
| 4-3472 | H | H | H | A28 | J1 | Q125 |
| 4-3473 | H | H | Cl | A28 | J1 | Q125 |
| 4-3474 | H | H | Br | A28 | J1 | Q125 |
| 4-3475 | H | H | CH₂F | A28 | J1 | Q125 |
| 4-3476 | H | H | CHF2 | A28 | J1 | Q125 |
| 4-3477 | H | H | Et | A28 | J1 | Q125 |
| 4-3478 | H | H | Pr | A28 | J1 | Q125 |
| 4-3479 | H | H | cPr | A28 | J1 | Q125 |
| 4-3480 | H | H | ethynyl | A28 | J1 | Q125 |
| 4-3481 | F | H | Me | A28 | J1 | Q125 |
| 4-3482 | F | F | Me | A28 | J1 | Q125 |
| 4-3483 | Me | H | Me | A28 | J1 | Q125 |
| 4-3484 | Me | Me | Me | A28 | J1 | Q125 |
| 4-3485 | H | H | H | A29 | J1 | Q125 |
| 4-3486 | H | H | Cl | A29 | J1 | Q125 |
| 4-3487 | H | H | Br | A29 | J1 | Q125 |
| 4-3488 | H | H | CH₂F | A29 | J1 | Q125 |
| 4-3489 | H | H | CHF2 | A29 | J1 | Q125 |
| 4-3490 | H | H | Et | A29 | J1 | Q125 |
| 4-3491 | H | H | Pr | A29 | J1 | Q125 |
| 4-3492 | H | H | cPr | A29 | J1 | Q125 |
| 4-3493 | H | H | ethynyl | A29 | J1 | Q125 |
| 4-3494 | F | H | Me | A29 | J1 | Q125 |
| 4-3495 | F | F | Me | A29 | J1 | Q125 |
| 4-3496 | Me | H | Me | A29 | J1 | Q125 |
| 4-3497 | Me | Me | Me | A29 | J1 | Q125 |
| 4-3498 | H | H | H | A30 | J1 | Q125 |
| 4-3499 | H | H | Cl | A30 | J1 | Q125 |
| 4-3500 | H | H | Br | A30 | J1 | Q125 |
| 4-3501 | H | H | CH₂F | A30 | J1 | Q125 |
| 4-3502 | H | H | CHF2 | A30 | J1 | Q125 |
| 4-3503 | H | H | Et | A30 | J1 | Q125 |
| 4-3504 | H | H | Pr | A30 | J1 | Q125 |
| 4-3505 | H | H | cPr | A30 | J1 | Q125 |
| 4-3506 | H | H | ethynyl | A30 | J1 | Q125 |
| 4-3507 | F | H | Me | A30 | J1 | Q125 |
| 4-3508 | F | F | Me | A30 | J1 | Q125 |
| 4-3509 | Me | H | Me | A30 | J1 | Q125 |
| 4-3510 | Me | Me | Me | A30 | J1 | Q125 |
| 4-3511 | H | H | H | A31 | J1 | Q125 |
| 4-3512 | H | H | Cl | A31 | J1 | Q125 |
| 4-3513 | H | H | Br | A31 | J1 | Q125 |
| 4-3514 | H | H | CH₂F | A31 | J1 | Q125 |
| 4-3515 | H | H | CHF2 | A31 | J1 | Q125 |
| 4-3516 | H | H | Et | A31 | J1 | Q125 |
| 4-3517 | H | H | Pr | A31 | J1 | Q125 |
| 4-3518 | H | H | cPr | A31 | J1 | Q125 |
| 4-3519 | H | H | ethynyl | A31 | J1 | Q125 |
| 4-3520 | F | H | Me | A31 | J1 | Q125 |
| 4-3521 | F | F | Me | A31 | J1 | Q125 |
| 4-3522 | Me | H | Me | A31 | J1 | Q125 |
| 4-3523 | Me | Me | Me | A31 | J1 | Q125 |
| 4-3524 | H | H | H | A51 | J1 | Q125 |
| 4-3525 | H | H | Cl | A51 | J1 | Q125 |
| 4-3526 | H | H | Br | A51 | J1 | Q125 |
| 4-3527 | H | H | CH₂F | A51 | J1 | Q125 |
| 4-3528 | H | H | CHF2 | A51 | J1 | Q125 |
| 4-3529 | H | H | Et | A51 | J1 | Q125 |
| 4-3530 | H | H | Pr | A51 | J1 | Q125 |
| 4-3531 | H | H | cPr | A51 | J1 | Q125 |
| 4-3532 | H | H | ethynyl | A51 | J1 | Q125 |
| 4-3533 | F | H | Me | A51 | J1 | Q125 |
| 4-3534 | F | F | Me | A51 | J1 | Q125 |
| 4-3535 | Me | H | Me | A51 | J1 | Q125 |
| 4-3536 | Me | Me | Me | A51 | J1 | Q125 |
| 4-3537 | H | H | H | A1 | J2 | Q150 |
| 4-3538 | H | H | Cl | A1 | J2 | Q150 |
| 4-3539 | H | H | Br | A1 | J2 | Q150 |
| 4-3540 | H | H | CH₂F | A1 | J2 | Q150 |
| 4-3541 | H | H | CHF2 | A1 | J2 | Q150 |
| 4-3542 | H | H | Et | A1 | J2 | Q150 |
| 4-3543 | H | H | Pr | A1 | J2 | Q150 |
| 4-3544 | H | H | cPr | A1 | J2 | Q150 |
| 4-3545 | H | H | ethynyl | A1 | J2 | Q150 |
| 4-3546 | F | H | Me | A1 | J2 | Q150 |
| 4-3547 | F | F | Me | A1 | J2 | Q150 |
| 4-3548 | Me | H | Me | A1 | J2 | Q150 |
| 4-3549 | Me | Me | Me | A1 | J2 | Q150 |
| 4-3550 | H | H | H | A2 | J2 | Q150 |
| 4-3551 | H | H | Cl | A2 | J2 | Q150 |
| 4-3552 | H | H | Br | A2 | J2 | Q150 |
| 4-3553 | H | H | CH₂F | A2 | J2 | Q150 |
| 4-3554 | H | H | CHF2 | A2 | J2 | Q150 |
| 4-3555 | H | H | Et | A2 | J2 | Q150 |
| 4-3556 | H | H | Pr | A2 | J2 | Q150 |
| 4-3557 | H | H | cPr | A2 | J2 | Q150 |
| 4-3558 | H | H | ethynyl | A2 | J2 | Q150 |
| 4-3559 | F | H | Me | A2 | J2 | Q150 |
| 4-3560 | F | F | Me | A2 | J2 | Q150 |
| 4-3561 | Me | H | Me | A2 | J2 | Q150 |
| 4-3562 | Me | Me | Me | A2 | J2 | Q150 |
| 4-3563 | H | H | H | A6 | J2 | Q150 |
| 4-3564 | H | H | Cl | A6 | J2 | Q150 |
| 4-3565 | H | H | Br | A6 | J2 | Q150 |
| 4-3566 | H | H | CH₂F | A6 | J2 | Q150 |
| 4-3567 | H | H | CHF2 | A6 | J2 | Q150 |
| 4-3568 | H | H | Et | A6 | J2 | Q150 |
| 4-3569 | H | H | Pr | A6 | J2 | Q150 |
| 4-3570 | H | H | cPr | A6 | J2 | Q150 |
| 4-3571 | H | H | ethynyl | A6 | J2 | Q150 |
| 4-3572 | F | H | Me | A6 | J2 | Q150 |
| 4-3573 | F | F | Me | A6 | J2 | Q150 |
| 4-3574 | Me | H | Me | A6 | J2 | Q150 |
| 4-3575 | Me | Me | Me | A6 | J2 | Q150 |
| 4-3576 | H | H | H | A9 | J2 | Q150 |
| 4-3577 | H | H | Cl | A9 | J2 | Q150 |
| 4-3578 | H | H | Br | A9 | J2 | Q150 |
| 4-3579 | H | H | CH₂F | A9 | J2 | Q150 |
| 4-3580 | H | H | CHF2 | A9 | J2 | Q150 |
| 4-3581 | H | H | Et | A9 | J2 | Q150 |
| 4-3582 | H | H | Pr | A9 | J2 | Q150 |
| 4-3583 | H | H | cPr | A9 | J2 | Q150 |
| 4-3584 | H | H | ethynyl | A9 | J2 | Q150 |
| 4-3585 | F | H | Me | A9 | J2 | Q150 |
| 4-3586 | F | F | Me | A9 | J2 | Q150 |
| 4-3587 | Me | H | Me | A9 | J2 | Q150 |
| 4-3588 | Me | Me | Me | A9 | J2 | Q150 |
| 4-3589 | H | H | H | A11 | J2 | Q150 |
| 4-3590 | H | H | Cl | A11 | J2 | Q150 |
| 4-3591 | H | H | Br | A11 | J2 | Q150 |
| 4-3592 | H | H | CH₂F | A11 | J2 | Q150 |
| 4-3593 | H | H | CHF2 | A11 | J2 | Q150 |
| 4-3594 | H | H | Et | A11 | J2 | Q150 |
| 4-3595 | H | H | Pr | A11 | J2 | Q150 |
| 4-3596 | H | H | cPr | A11 | J2 | Q150 |
| 4-3597 | H | H | ethynyl | A11 | J2 | Q150 |
| 4-3598 | F | H | Me | A11 | J2 | Q150 |
| 4-3599 | F | F | Me | A11 | J2 | Q150 |
| 4-3600 | Me | H | Me | A11 | J2 | Q150 |
| 4-3601 | Me | Me | Me | A11 | J2 | Q150 |
| 4-3602 | H | H | H | A12 | J2 | Q150 |
| 4-3603 | H | H | Cl | A12 | J2 | Q150 |
| 4-3604 | H | H | Br | A12 | J2 | Q150 |
| 4-3605 | H | H | CH₂F | A12 | J2 | Q150 |
| 4-3606 | H | H | CHF2 | A12 | J2 | Q150 |
| 4-3607 | H | H | Et | A12 | J2 | Q150 |
| 4-3608 | H | H | Pr | A12 | J2 | Q150 |
| 4-3609 | H | H | cPr | A12 | J2 | Q150 |
| 4-3610 | H | H | ethynyl | A12 | J2 | Q150 |
| 4-3611 | F | H | Me | A12 | J2 | Q150 |
| 4-3612 | F | F | Me | A12 | J2 | Q150 |
| 4-3613 | Me | H | Me | A12 | J2 | Q150 |
| 4-3614 | Me | Me | Me | A12 | J2 | Q150 |
| 4-3615 | H | H | H | A19 | J2 | Q150 |
| 4-3616 | H | H | Cl | A19 | J2 | Q150 |
| 4-3617 | H | H | Br | A19 | J2 | Q150 |
| 4-3618 | H | H | CH₂F | A19 | J2 | Q150 |
| 4-3619 | H | H | CHF2 | A19 | J2 | Q150 |
| 4-3620 | H | H | Et | A19 | J2 | Q150 |
| 4-3621 | H | H | Pr | A19 | J2 | Q150 |
| 4-3622 | H | H | cPr | A19 | J2 | Q150 |
| 4-3623 | H | H | ethynyl | A19 | J2 | Q150 |
| 4-3624 | F | H | Me | A19 | J2 | Q150 |
| 4-3625 | F | F | Me | A19 | J2 | Q150 |
| 4-3626 | Me | H | Me | A19 | J2 | Q150 |
| 4-3627 | Me | Me | Me | A19 | J2 | Q150 |
| 4-3628 | H | H | H | A20 | J2 | Q150 |
| 4-3629 | H | H | Cl | A20 | J2 | Q150 |
| 4-3630 | H | H | Br | A20 | J2 | Q150 |
| 4-3631 | H | H | CH₂F | A20 | J2 | Q150 |
| 4-3632 | H | H | CHF2 | A20 | J2 | Q150 |
| 4-3633 | H | H | Et | A20 | J2 | Q150 |
| 4-3634 | H | H | Pr | A20 | J2 | Q150 |
| 4-3635 | H | H | cPr | A20 | J2 | Q150 |
| 4-3636 | H | H | ethynyl | A20 | J2 | Q150 |
| 4-3637 | F | H | Me | A20 | J2 | Q150 |
| 4-3638 | F | F | Me | A20 | J2 | Q150 |
| 4-3639 | Me | H | Me | A20 | J2 | Q150 |
| 4-3640 | Me | Me | Me | A20 | J2 | Q150 |
| 4-3641 | H | H | H | A21 | J2 | Q150 |
| 4-3642 | H | H | Cl | A21 | J2 | Q150 |
| 4-3643 | H | H | Br | A21 | J2 | Q150 |
| 4-3644 | H | H | CH₂F | A21 | J2 | Q150 |
| 4-3645 | H | H | CHF2 | A21 | J2 | Q150 |
| 4-3646 | H | H | Et | A21 | J2 | Q150 |
| 4-3647 | H | H | Pr | A21 | J2 | Q150 |
| 4-3648 | H | H | cPr | A21 | J2 | Q150 |
| 4-3649 | H | H | ethynyl | A21 | J2 | Q150 |
| 4-3650 | F | H | Me | A21 | J2 | Q150 |
| 4-3651 | F | F | Me | A21 | J2 | Q150 |
| 4-3652 | Me | H | Me | A21 | J2 | Q150 |
| 4-3653 | Me | Me | Me | A21 | J2 | Q150 |
| 4-3654 | H | H | H | A22 | J2 | Q150 |
| 4-3655 | H | H | Cl | A22 | J2 | Q150 |
| 4-3656 | H | H | Br | A22 | J2 | Q150 |
| 4-3657 | H | H | CH₂F | A22 | J2 | Q150 |
| 4-3658 | H | H | CHF2 | A22 | J2 | Q150 |
| 4-3659 | H | H | Et | A22 | J2 | Q150 |
| 4-3660 | H | H | Pr | A22 | J2 | Q150 |
| 4-3661 | H | H | cPr | A22 | J2 | Q150 |
| 4-3662 | H | H | ethynyl | A22 | J2 | Q150 |
| 4-3663 | F | H | Me | A22 | J2 | Q150 |
| 4-3664 | F | F | Me | A22 | J2 | Q150 |
| 4-3665 | Me | H | Me | A22 | J2 | Q150 |
| 4-3666 | Me | Me | Me | A22 | J2 | Q150 |
| 4-3667 | H | H | H | A26 | J2 | Q150 |
| 4-3668 | H | H | Cl | A26 | J2 | Q150 |
| 4-3669 | H | H | Br | A26 | J2 | Q150 |
| 4-3670 | H | H | CH₂F | A26 | J2 | Q150 |
| 4-3671 | H | H | CHF2 | A26 | J2 | Q150 |
| 4-3672 | H | H | Et | A26 | J2 | Q150 |
| 4-3673 | H | H | Pr | A26 | J2 | Q150 |
| 4-3674 | H | H | cPr | A26 | J2 | Q150 |
| 4-3675 | H | H | ethynyl | A26 | J2 | Q150 |
| 4-3676 | F | H | Me | A26 | J2 | Q150 |
| 4-3677 | F | F | Me | A26 | J2 | Q150 |
| 4-3678 | Me | H | Me | A26 | J2 | Q150 |
| 4-3679 | Me | Me | Me | A26 | J2 | Q150 |
| 4-3680 | H | H | H | A27 | J2 | Q150 |
| 4-3681 | H | H | Cl | A27 | J2 | Q150 |
| 4-3682 | H | H | Br | A27 | J2 | Q150 |
| 4-3683 | H | H | CH₂F | A27 | J2 | Q150 |
| 4-3684 | H | H | CHF2 | A27 | J2 | Q150 |
| 4-3685 | H | H | Et | A27 | J2 | Q150 |
| 4-3686 | H | H | Pr | A27 | J2 | Q150 |
| 4-3687 | H | H | cPr | A27 | J2 | Q150 |
| 4-3688 | H | H | ethynyl | A27 | J2 | Q150 |
| 4-3689 | F | H | Me | A27 | J2 | Q150 |
| 4-3690 | F | F | Me | A27 | J2 | Q150 |
| 4-3691 | Me | H | Me | A27 | J2 | Q150 |
| 4-3692 | Me | Me | Me | A27 | J2 | Q150 |
| 4-3693 | H | H | H | A28 | J2 | Q150 |
| 4-3694 | H | H | Cl | A28 | J2 | Q150 |
| 4-3695 | H | H | Br | A28 | J2 | Q150 |
| 4-3696 | H | H | CH₂F | A28 | J2 | Q150 |
| 4-3697 | H | H | CHF2 | A28 | J2 | Q150 |
| 4-3698 | H | H | Et | A28 | J2 | Q150 |
| 4-3699 | H | H | Pr | A28 | J2 | Q150 |
| 4-3700 | H | H | cPr | A28 | J2 | Q150 |
| 4-3701 | H | H | ethynyl | A28 | J2 | Q150 |
| 4-3702 | F | H | Me | A28 | J2 | Q150 |
| 4-3703 | F | F | Me | A28 | J2 | Q150 |
| 4-3704 | Me | H | Me | A28 | J2 | Q150 |
| 4-3705 | Me | Me | Me | A28 | J2 | Q150 |
| 4-3706 | H | H | H | A29 | J2 | Q150 |
| 4-3707 | H | H | Cl | A29 | J2 | Q150 |
| 4-3708 | H | H | Br | A29 | J2 | Q150 |
| 4-3709 | H | H | CH₂F | A29 | J2 | Q150 |
| 4-3710 | H | H | CHF2 | A29 | J2 | Q150 |
| 4-3711 | H | H | Et | A29 | J2 | Q150 |
| 4-3712 | H | H | Pr | A29 | J2 | Q150 |
| 4-3713 | H | H | cPr | A29 | J2 | Q150 |
| 4-3714 | H | H | ethynyl | A29 | J2 | Q150 |
| 4-3715 | F | H | Me | A29 | J2 | Q150 |
| 4-3716 | F | F | Me | A29 | J2 | Q150 |
| 4-3717 | Me | H | Me | A29 | J2 | Q150 |
| 4-3718 | Me | Me | Me | A29 | J2 | Q150 |
| 4-3719 | H | H | H | A30 | J2 | Q150 |
| 4-3720 | H | H | Cl | A30 | J2 | Q150 |
| 4-3721 | H | H | Br | A30 | J2 | Q150 |
| 4-3722 | H | H | CH₂F | A30 | J2 | Q150 |
| 4-3723 | H | H | CHF2 | A30 | J2 | Q150 |
| 4-3724 | H | H | Et | A30 | J2 | Q150 |
| 4-3725 | H | H | Pr | A30 | J2 | Q150 |
| 4-3726 | H | H | cPr | A30 | J2 | Q150 |
| 4-3727 | H | H | ethynyl | A30 | J2 | Q150 |
| 4-3728 | F | H | Me | A30 | J2 | Q150 |
| 4-3729 | F | F | Me | A30 | J2 | Q150 |
| 4-3730 | Me | H | Me | A30 | J2 | Q150 |
| 4-3731 | Me | Me | Me | A30 | J2 | Q150 |
| 4-3732 | H | H | H | A31 | J2 | Q150 |
| 4-3733 | H | H | Cl | A31 | J2 | Q150 |
| 4-3734 | H | H | Br | A31 | J2 | Q150 |
| 4-3735 | H | H | CH₂F | A31 | J2 | Q150 |
| 4-3736 | H | H | CHF2 | A31 | J2 | Q150 |
| 4-3737 | H | H | Et | A31 | J2 | Q150 |
| 4-3738 | H | H | Pr | A31 | J2 | Q150 |
| 4-3739 | H | H | cPr | A31 | J2 | Q150 |
| 4-3740 | H | H | ethynyl | A31 | J2 | Q150 |
| 4-3741 | F | H | Me | A31 | J2 | Q150 |
| 4-3742 | F | F | Me | A31 | J2 | Q150 |
| 4-3743 | Me | H | Me | A31 | J2 | Q150 |
| 4-3744 | Me | Me | Me | A31 | J2 | Q150 |
| 4-3745 | H | H | H | A51 | J2 | Q150 |
| 4-3746 | H | H | Cl | A51 | J2 | Q150 |
| 4-3747 | H | H | Br | A51 | J2 | Q150 |
| 4-3748 | H | H | CH₂F | A51 | J2 | Q150 |
| 4-3749 | H | H | CHF2 | A51 | J2 | Q150 |
| 4-3750 | H | H | Et | A51 | J2 | Q150 |
| 4-3751 | H | H | Pr | A51 | J2 | Q150 |
| 4-3752 | H | H | cPr | A51 | J2 | Q150 |
| 4-3753 | H | H | ethynyl | A51 | J2 | Q150 |
| 4-3754 | F | H | Me | A51 | J2 | Q150 |
| 4-3755 | F | F | Me | A51 | J2 | Q150 |
| 4-3756 | Me | H | Me | A51 | J2 | Q150 |
| 4-3757 | Me | Me | Me | A51 | J2 | Q150 |
| 4-3758 | H | H | H | A1 | J1 | Q150 |
| 4-3759 | H | H | Cl | A1 | J1 | Q150 |
| 4-3760 | H | H | Br | A1 | J1 | Q150 |
| 4-3761 | H | H | CH₂F | A1 | J1 | Q150 |
| 4-3762 | H | H | CHF2 | A1 | J1 | Q150 |
| 4-3763 | H | H | Et | A1 | J1 | Q150 |
| 4-3764 | H | H | Pr | A1 | J1 | Q150 |
| 4-3765 | H | H | cPr | A1 | J1 | Q150 |
| 4-3766 | H | H | ethynyl | A1 | J1 | Q150 |
| 4-3767 | F | H | Me | A1 | J1 | Q150 |
| 4-3768 | F | F | Me | A1 | J1 | Q150 |
| 4-3769 | Me | H | Me | A1 | J1 | Q150 |
| 4-3770 | Me | Me | Me | A1 | J1 | Q150 |
| 4-3771 | H | H | H | A2 | J1 | Q150 |
| 4-3772 | H | H | Cl | A2 | J1 | Q150 |
| 4-3773 | H | H | Br | A2 | J1 | Q150 |
| 4-3774 | H | H | CH₂F | A2 | J1 | Q150 |
| 4-3775 | H | H | CHF2 | A2 | J1 | Q150 |
| 4-3776 | H | H | Et | A2 | J1 | Q150 |
| 4-3777 | H | H | Pr | A2 | J1 | Q150 |
| 4-3778 | H | H | cPr | A2 | J1 | Q150 |
| 4-3779 | H | H | ethynyl | A2 | J1 | Q150 |
| 4-3780 | F | H | Me | A2 | J1 | Q150 |
| 4-3781 | F | F | Me | A2 | J1 | Q150 |
| 4-3782 | Me | H | Me | A2 | J1 | Q150 |
| 4-3783 | Me | Me | Me | A2 | J1 | Q150 |
| 4-3784 | H | H | H | A6 | J1 | Q150 |
| 4-3785 | H | H | Cl | A6 | J1 | Q150 |
| 4-3786 | H | H | Br | A6 | J1 | Q150 |
| 4-3787 | H | H | CH₂F | A6 | J1 | Q150 |
| 4-3788 | H | H | CHF2 | A6 | J1 | Q150 |
| 4-3789 | H | H | Et | A6 | J1 | Q150 |
| 4-3790 | H | H | Pr | A6 | J1 | Q150 |
| 4-3791 | H | H | cPr | A6 | J1 | Q150 |
| 4-3792 | H | H | ethynyl | A6 | J1 | Q150 |
| 4-3793 | F | H | Me | A6 | J1 | Q150 |
| 4-3794 | F | F | Me | A6 | J1 | Q150 |
| 4-3795 | Me | H | Me | A6 | J1 | Q150 |
| 4-3796 | Me | Me | Me | A6 | J1 | Q150 |
| 4-3797 | H | H | H | A9 | J1 | Q150 |
| 4-3798 | H | H | Cl | A9 | J1 | Q150 |
| 4-3799 | H | H | Br | A9 | J1 | Q150 |
| 4-3800 | H | H | CH₂F | A9 | J1 | Q150 |
| 4-3801 | H | H | CHF2 | A9 | J1 | Q150 |
| 4-3802 | H | H | Et | A9 | J1 | Q150 |
| 4-3803 | H | H | Pr | A9 | J1 | Q150 |
| 4-3804 | H | H | cPr | A9 | J1 | Q150 |
| 4-3805 | H | H | ethynyl | A9 | J1 | Q150 |
| 4-3806 | F | H | Me | A9 | J1 | Q150 |
| 4-3807 | F | F | Me | A9 | J1 | Q150 |
| 4-3808 | Me | H | Me | A9 | J1 | Q150 |
| 4-3809 | Me | Me | Me | A9 | J1 | Q150 |
| 4-3810 | H | H | H | A11 | J1 | Q150 |
| 4-3811 | H | H | Cl | A11 | J1 | Q150 |
| 4-3812 | H | H | Br | A11 | J1 | Q150 |
| 4-3813 | H | H | CH₂F | A11 | J1 | Q150 |
| 4-3814 | H | H | CHF2 | A11 | J1 | Q150 |
| 4-3815 | H | H | Et | A11 | J1 | Q150 |
| 4-3816 | H | H | Pr | A11 | J1 | Q150 |
| 4-3817 | H | H | cPr | A11 | J1 | Q150 |
| 4-3818 | H | H | ethynyl | A11 | J1 | Q150 |
| 4-3819 | F | H | Me | A11 | J1 | Q150 |
| 4-3820 | F | F | Me | A11 | J1 | Q150 |
| 4-3821 | Me | H | Me | A11 | J1 | Q150 |
| 4-3822 | Me | Me | Me | A11 | J1 | Q150 |
| 4-3823 | H | H | H | A12 | J1 | Q150 |
| 4-3824 | H | H | Cl | A12 | J1 | Q150 |
| 4-3825 | H | H | Br | A12 | J1 | Q150 |
| 4-3826 | H | H | CH₂F | A12 | J1 | Q150 |
| 4-3827 | H | H | CHF2 | A12 | J1 | Q150 |
| 4-3828 | H | H | Et | A12 | J1 | Q150 |
| 4-3829 | H | H | Pr | A12 | J1 | Q150 |
| 4-3830 | H | H | cPr | A12 | J1 | Q150 |
| 4-3831 | H | H | ethynyl | A12 | J1 | Q150 |
| 4-3832 | F | H | Me | A12 | J1 | Q150 |
| 4-3833 | F | F | Me | A12 | J1 | Q150 |
| 4-3834 | Me | H | Me | A12 | J1 | Q150 |
| 4-3835 | Me | Me | Me | A12 | J1 | Q150 |
| 4-3836 | H | H | H | A19 | J1 | Q150 |
| 4-3837 | H | H | Cl | A19 | J1 | Q150 |
| 4-3838 | H | H | Br | A19 | J1 | Q150 |
| 4-3839 | H | H | CH₂F | A19 | J1 | Q150 |
| 4-3840 | H | H | CHF2 | A19 | J1 | Q150 |
| 4-3841 | H | H | Et | A19 | J1 | Q150 |
| 4-3842 | H | H | Pr | A19 | J1 | Q150 |
| 4-3843 | H | H | cPr | A19 | J1 | Q150 |
| 4-3844 | H | H | ethynyl | A19 | J1 | Q150 |
| 4-3845 | F | H | Me | A19 | J1 | Q150 |
| 4-3846 | F | F | Me | A19 | J1 | Q150 |
| 4-3847 | Me | H | Me | A19 | J1 | Q150 |
| 4-3848 | Me | Me | Me | A19 | J1 | Q150 |
| 4-3849 | H | H | H | A20 | J1 | Q150 |
| 4-3850 | H | H | Cl | A20 | J1 | Q150 |
| 4-3851 | H | H | Br | A20 | J1 | Q150 |
| 4-3852 | H | H | CH₂F | A20 | J1 | Q150 |
| 4-3853 | H | H | CHF2 | A20 | J1 | Q150 |
| 4-3854 | H | H | Et | A20 | J1 | Q150 |
| 4-3855 | H | H | Pr | A20 | J1 | Q150 |
| 4-3856 | H | H | cPr | A20 | J1 | Q150 |
| 4-3857 | H | H | ethynyl | A20 | J1 | Q150 |
| 4-3858 | F | H | Me | A20 | J1 | Q150 |
| 4-3859 | F | F | Me | A20 | J1 | Q150 |
| 4-3860 | Me | H | Me | A20 | J1 | Q150 |
| 4-3861 | Me | Me | Me | A20 | J1 | Q150 |
| 4-3862 | H | H | H | A21 | J1 | Q150 |
| 4-3863 | H | H | Cl | A21 | J1 | Q150 |
| 4-3864 | H | H | Br | A21 | J1 | Q150 |
| 4-3865 | H | H | CH₂F | A21 | J1 | Q150 |
| 4-3866 | H | H | CHF2 | A21 | J1 | Q150 |
| 4-3867 | H | H | Et | A21 | J1 | Q150 |
| 4-3868 | H | H | Pr | A21 | J1 | Q150 |
| 4-3869 | H | H | cPr | A21 | J1 | Q150 |
| 4-3870 | H | H | ethynyl | A21 | J1 | Q150 |
| 4-3871 | F | H | Me | A21 | J1 | Q150 |
| 4-3872 | F | F | Me | A21 | J1 | Q150 |
| 4-3873 | Me | H | Me | A21 | J1 | Q150 |
| 4-3874 | Me | Me | Me | A21 | J1 | Q150 |
| 4-3875 | H | H | H | A22 | J1 | Q150 |
| 4-3876 | H | H | Cl | A22 | J1 | Q150 |
| 4-3877 | H | H | Br | A22 | J1 | Q150 |
| 4-3878 | H | H | CH₂F | A22 | J1 | Q150 |
| 4-3879 | H | H | CHF2 | A22 | J1 | Q150 |
| 4-3880 | H | H | Et | A22 | J1 | Q150 |
| 4-3881 | H | H | Pr | A22 | J1 | Q150 |
| 4-3882 | H | H | cPr | A22 | J1 | Q150 |
| 4-3883 | H | H | ethynyl | A22 | J1 | Q150 |
| 4-3884 | F | H | Me | A22 | J1 | Q150 |
| 4-3885 | F | F | Me | A22 | J1 | Q150 |
| 4-3886 | Me | H | Me | A22 | J1 | Q150 |
| 4-3887 | Me | Me | Me | A22 | J1 | Q150 |
| 4-3888 | H | H | H | A26 | J1 | Q150 |
| 4-3889 | H | H | Cl | A26 | J1 | Q150 |
| 4-3890 | H | H | Br | A26 | J1 | Q150 |
| 4-3891 | H | H | CH₂F | A26 | J1 | Q150 |
| 4-3892 | H | H | CHF2 | A26 | J1 | Q150 |
| 4-3893 | H | H | Et | A26 | J1 | Q150 |
| 4-3894 | H | H | Pr | A26 | J1 | Q150 |
| 4-3895 | H | H | cPr | A26 | J1 | Q150 |
| 4-3896 | H | H | ethynyl | A26 | J1 | Q150 |
| 4-3897 | F | H | Me | A26 | J1 | Q150 |
| 4-3898 | F | F | Me | A26 | J1 | Q150 |
| 4-3899 | Me | H | Me | A26 | J1 | Q150 |
| 4-3900 | Me | Me | Me | A26 | J1 | Q150 |
| 4-3901 | H | H | H | A27 | J1 | Q150 |
| 4-3902 | H | H | Cl | A27 | J1 | Q150 |
| 4-3903 | H | H | Br | A27 | J1 | Q150 |
| 4-3904 | H | H | CH₂F | A27 | J1 | Q150 |
| 4-3905 | H | H | CHF2 | A27 | J1 | Q150 |
| 4-3906 | H | H | Et | A27 | J1 | Q150 |
| 4-3907 | H | H | Pr | A27 | J1 | Q150 |
| 4-3908 | H | H | cPr | A27 | J1 | Q150 |
| 4-3909 | H | H | ethynyl | A27 | J1 | Q150 |
| 4-3910 | F | H | Me | A27 | J1 | Q150 |
| 4-3911 | F | F | Me | A27 | J1 | Q150 |
| 4-3912 | Me | H | Me | A27 | J1 | Q150 |
| 4-3913 | Me | Me | Me | A27 | J1 | Q150 |
| 4-3914 | H | H | H | A28 | J1 | Q150 |
| 4-3915 | H | H | Cl | A28 | J1 | Q150 |
| 4-3916 | H | H | Br | A28 | J1 | Q150 |
| 4-3917 | H | H | CH₂F | A28 | J1 | Q150 |
| 4-3918 | H | H | CHF2 | A28 | J1 | Q150 |
| 4-3919 | H | H | Et | A28 | J1 | Q150 |
| 4-3920 | H | H | Pr | A28 | J1 | Q150 |
| 4-3921 | H | H | cPr | A28 | J1 | Q150 |
| 4-3922 | H | H | ethynyl | A28 | J1 | Q150 |
| 4-3923 | F | H | Me | A28 | J1 | Q150 |
| 4-3924 | F | F | Me | A28 | J1 | Q150 |
| 4-3925 | Me | H | Me | A28 | J1 | Q150 |
| 4-3926 | Me | Me | Me | A28 | J1 | Q150 |
| 4-3927 | H | H | H | A29 | J1 | Q150 |
| 4-3928 | H | H | Cl | A29 | J1 | Q150 |
| 4-3929 | H | H | Br | A29 | J1 | Q150 |
| 4-3930 | H | H | CH₂F | A29 | J1 | Q150 |
| 4-3931 | H | H | CHF2 | A29 | J1 | Q150 |
| 4-3932 | H | H | Et | A29 | J1 | Q150 |
| 4-3933 | H | H | Pr | A29 | J1 | Q150 |
| 4-3934 | H | H | cPr | A29 | J1 | Q150 |
| 4-3935 | H | H | ethynyl | A29 | J1 | Q150 |
| 4-3936 | F | H | Me | A29 | J1 | Q150 |
| 4-3937 | F | F | Me | A29 | J1 | Q150 |
| 4-3938 | Me | H | Me | A29 | J1 | Q150 |
| 4-3939 | Me | Me | Me | A29 | J1 | Q150 |
| 4-3940 | H | H | H | A30 | J1 | Q150 |
| 4-3941 | H | H | Cl | A30 | J1 | Q150 |
| 4-3942 | H | H | Br | A30 | J1 | Q150 |
| 4-3943 | H | H | CH₂F | A30 | J1 | Q150 |
| 4-3944 | H | H | CHF2 | A30 | J1 | Q150 |
| 4-3945 | H | H | Et | A30 | J1 | Q150 |
| 4-3946 | H | H | Pr | A30 | J1 | Q150 |
| 4-3947 | H | H | cPr | A30 | J1 | Q150 |
| 4-3948 | H | H | ethynyl | A30 | J1 | Q150 |
| 4-3949 | F | H | Me | A30 | J1 | Q150 |
| 4-3950 | F | F | Me | A30 | J1 | Q150 |
| 4-3951 | Me | H | Me | A30 | J1 | Q150 |
| 4-3952 | Me | Me | Me | A30 | J1 | Q150 |
| 4-3953 | H | H | H | A31 | J1 | Q150 |
| 4-3954 | H | H | Cl | A31 | J1 | Q150 |
| 4-3955 | H | H | Br | A31 | J1 | Q150 |
| 4-3956 | H | H | CH₂F | A31 | J1 | Q150 |
| 4-3957 | H | H | CHF2 | A31 | J1 | Q150 |
| 4-3958 | H | H | Et | A31 | J1 | Q150 |
| 4-3959 | H | H | Pr | A31 | J1 | Q150 |
| 4-3960 | H | H | cPr | A31 | J1 | Q150 |
| 4-3961 | H | H | ethynyl | A31 | J1 | Q150 |
| 4-3962 | F | H | Me | A31 | J1 | Q150 |
| 4-3963 | F | F | Me | A31 | J1 | Q150 |
| 4-3964 | Me | H | Me | A31 | J1 | Q150 |
| 4-3965 | Me | Me | Me | A31 | J1 | Q150 |
| 4-3966 | H | H | H | A51 | J1 | Q150 |
| 4-3967 | H | H | Cl | A51 | J1 | Q150 |
| 4-3968 | H | H | Br | A51 | J1 | Q150 |
| 4-3969 | H | H | CH₂F | A51 | J1 | Q150 |
| 4-3970 | H | H | CHF2 | A51 | J1 | Q150 |
| 4-3971 | H | H | Et | A51 | J1 | Q150 |
| 4-3972 | H | H | Pr | A51 | J1 | Q150 |
| 4-3973 | H | H | cPr | A51 | J1 | Q150 |
| 4-3974 | H | H | ethynyl | A51 | J1 | Q150 |
| 4-3975 | F | H | Me | A51 | J1 | Q150 |
| 4-3976 | F | F | Me | A51 | J1 | Q150 |
| 4-3977 | Me | H | Me | A51 | J1 | Q150 |
| 4-3978 | Me | Me | Me | A51 | J1 | Q150 |
| 4-3979 | H | H | H | A1 | J2 | Q152 |
| 4-3980 | H | H | Cl | A1 | J2 | Q152 |
| 4-3981 | H | H | Br | A1 | J2 | Q152 |
| 4-3982 | H | H | CH₂F | A1 | J2 | Q152 |
| 4-3983 | H | H | CHF2 | A1 | J2 | Q152 |
| 4-3984 | H | H | Et | A1 | J2 | Q152 |
| 4-3985 | H | H | Pr | A1 | J2 | Q152 |
| 4-3986 | H | H | cPr | A1 | J2 | Q152 |
| 4-3987 | H | H | ethynyl | A1 | J2 | Q152 |
| 4-3988 | F | H | Me | A1 | J2 | Q152 |
| 4-3989 | F | F | Me | A1 | J2 | Q152 |
| 4-3990 | Me | H | Me | A1 | J2 | Q152 |
| 4-3991 | Me | Me | Me | A1 | J2 | Q152 |
| 4-3992 | H | H | H | A2 | J2 | Q152 |
| 4-3993 | H | H | Cl | A2 | J2 | Q152 |
| 4-3994 | H | H | Br | A2 | J2 | Q152 |
| 4-3995 | H | H | CH₂F | A2 | J2 | Q152 |
| 4-3996 | H | H | CHF2 | A2 | J2 | Q152 |
| 4-3997 | H | H | Et | A2 | J2 | Q152 |
| 4-3998 | H | H | Pr | A2 | J2 | Q152 |
| 4-3999 | H | H | cPr | A2 | J2 | Q152 |
| 4-4000 | H | H | ethynyl | A2 | J2 | Q152 |
| 4-4001 | F | H | Me | A2 | J2 | Q152 |
| 4-4002 | F | F | Me | A2 | J2 | Q152 |
| 4-4003 | Me | H | Me | A2 | J2 | Q152 |
| 4-4004 | Me | Me | Me | A2 | J2 | Q152 |
| 4-4005 | H | H | H | A6 | J2 | Q152 |
| 4-4006 | H | H | Cl | A6 | J2 | Q152 |
| 4-4007 | H | H | Br | A6 | J2 | Q152 |
| 4-4008 | H | H | CH₂F | A6 | J2 | Q152 |
| 4-4009 | H | H | CHF2 | A6 | J2 | Q152 |
| 4-4010 | H | H | Et | A6 | J2 | Q152 |
| 4-4011 | H | H | Pr | A6 | J2 | Q152 |
| 4-4012 | H | H | cPr | A6 | J2 | Q152 |
| 4-4013 | H | H | ethynyl | A6 | J2 | Q152 |
| 4-4014 | F | H | Me | A6 | J2 | Q152 |
| 4-4015 | F | F | Me | A6 | J2 | Q152 |
| 4-4016 | Me | H | Me | A6 | J2 | Q152 |
| 4-4017 | Me | Me | Me | A6 | J2 | Q152 |
| 4-4018 | H | H | H | A9 | J2 | Q152 |
| 4-4019 | H | H | Cl | A9 | J2 | Q152 |
| 4-4020 | H | H | Br | A9 | J2 | Q152 |
| 4-4021 | H | H | CH₂F | A9 | J2 | Q152 |
| 4-4022 | H | H | CHF2 | A9 | J2 | Q152 |
| 4-4023 | H | H | Et | A9 | J2 | Q152 |
| 4-4024 | H | H | Pr | A9 | J2 | Q152 |
| 4-4025 | H | H | cPr | A9 | J2 | Q152 |
| 4-4026 | H | H | ethynyl | A9 | J2 | Q152 |
| 4-4027 | F | H | Me | A9 | J2 | Q152 |
| 4-4028 | F | F | Me | A9 | J2 | Q152 |
| 4-4029 | Me | H | Me | A9 | J2 | Q152 |
| 4-4030 | Me | Me | Me | A9 | J2 | Q152 |
| 4-4031 | H | H | H | A11 | J2 | Q152 |
| 4-4032 | H | H | Cl | A11 | J2 | Q152 |
| 4-4033 | H | H | Br | A11 | J2 | Q152 |
| 4-4034 | H | H | CH₂F | A11 | J2 | Q152 |
| 4-4035 | H | H | CHF2 | A11 | J2 | Q152 |
| 4-4036 | H | H | Et | A11 | J2 | Q152 |
| 4-4037 | H | H | Pr | A11 | J2 | Q152 |
| 4-4038 | H | H | cPr | A11 | J2 | Q152 |
| 4-4039 | H | H | ethynyl | A11 | J2 | Q152 |
| 4-4040 | F | H | Me | A11 | J2 | Q152 |
| 4-4041 | F | F | Me | A11 | J2 | Q152 |
| 4-4042 | Me | H | Me | A11 | J2 | Q152 |
| 4-4043 | Me | Me | Me | A11 | J2 | Q152 |
| 4-4044 | H | H | H | A12 | J2 | Q152 |
| 4-4045 | H | H | Cl | A12 | J2 | Q152 |
| 4-4046 | H | H | Br | A12 | J2 | Q152 |
| 4-4047 | H | H | CH₂F | A12 | J2 | Q152 |
| 4-4048 | H | H | CHF2 | A12 | J2 | Q152 |
| 4-4049 | H | H | Et | A12 | J2 | Q152 |
| 4-4050 | H | H | Pr | A12 | J2 | Q152 |
| 4-4051 | H | H | cPr | A12 | J2 | Q152 |
| 4-4052 | H | H | ethynyl | A12 | J2 | Q152 |
| 4-4053 | F | H | Me | A12 | J2 | Q152 |
| 4-4054 | F | F | Me | A12 | J2 | Q152 |
| 4-4055 | Me | H | Me | A12 | J2 | Q152 |
| 4-4056 | Me | Me | Me | A12 | J2 | Q152 |
| 4-4057 | H | H | H | A19 | J2 | Q152 |
| 4-4058 | H | H | Cl | A19 | J2 | Q152 |
| 4-4059 | H | H | Br | A19 | J2 | Q152 |
| 4-4060 | H | H | CH₂F | A19 | J2 | Q152 |
| 4-4061 | H | H | CHF2 | A19 | J2 | Q152 |
| 4-4062 | H | H | Et | A19 | J2 | Q152 |
| 4-4063 | H | H | Pr | A19 | J2 | Q152 |
| 4-4064 | H | H | cPr | A19 | J2 | Q152 |
| 4-4065 | H | H | ethynyl | A19 | J2 | Q152 |
| 4-4066 | F | H | Me | A19 | J2 | Q152 |
| 4-4067 | F | F | Me | A19 | J2 | Q152 |
| 4-4068 | Me | H | Me | A19 | J2 | Q152 |
| 4-4069 | Me | Me | Me | A19 | J2 | Q152 |
| 4-4070 | H | H | H | A20 | J2 | Q152 |
| 4-4071 | H | H | Cl | A20 | J2 | Q152 |
| 4-4072 | H | H | Br | A20 | J2 | Q152 |
| 4-4073 | H | H | CH₂F | A20 | J2 | Q152 |
| 4-4074 | H | H | CHF2 | A20 | J2 | Q152 |
| 4-4075 | H | H | Et | A20 | J2 | Q152 |
| 4-4076 | H | H | Pr | A20 | J2 | Q152 |
| 4-4077 | H | H | cPr | A20 | J2 | Q152 |
| 4-4078 | H | H | ethynyl | A20 | J2 | Q152 |
| 4-4079 | F | H | Me | A20 | J2 | Q152 |
| 4-4080 | F | F | Me | A20 | J2 | Q152 |
| 4-4081 | Me | H | Me | A20 | J2 | Q152 |
| 4-4082 | Me | Me | Me | A20 | J2 | Q152 |
| 4-4083 | H | H | H | A21 | J2 | Q152 |
| 4-4084 | H | H | Cl | A21 | J2 | Q152 |
| 4-4085 | H | H | Br | A21 | J2 | Q152 |
| 4-4086 | H | H | CH₂F | A21 | J2 | Q152 |
| 4-4087 | H | H | CHF2 | A21 | J2 | Q152 |
| 4-4088 | H | H | Et | A21 | J2 | Q152 |
| 4-4089 | H | H | Pr | A21 | J2 | Q152 |
| 4-4090 | H | H | cPr | A21 | J2 | Q152 |
| 4-4091 | H | H | ethynyl | A21 | J2 | Q152 |
| 4-4092 | F | H | Me | A21 | J2 | Q152 |
| 4-4093 | F | F | Me | A21 | J2 | Q152 |
| 4-4094 | Me | H | Me | A21 | J2 | Q152 |
| 4-4095 | Me | Me | Me | A21 | J2 | Q152 |
| 4-4096 | H | H | H | A22 | J2 | Q152 |
| 4-4097 | H | H | Cl | A22 | J2 | Q152 |
| 4-4098 | H | H | Br | A22 | J2 | Q152 |
| 4-4099 | H | H | CH₂F | A22 | J2 | Q152 |
| 4-4100 | H | H | CHF2 | A22 | J2 | Q152 |
| 4-4101 | H | H | Et | A22 | J2 | Q152 |
| 4-4102 | H | H | Pr | A22 | J2 | Q152 |
| 4-4103 | H | H | cPr | A22 | J2 | Q152 |
| 4-4104 | H | H | ethynyl | A22 | J2 | Q152 |
| 4-4105 | F | H | Me | A22 | J2 | Q152 |
| 4-4106 | F | F | Me | A22 | J2 | Q152 |
| 4-4107 | Me | H | Me | A22 | J2 | Q152 |
| 4-4108 | Me | Me | Me | A22 | J2 | Q152 |
| 4-4109 | H | H | H | A26 | J2 | Q152 |
| 4-4110 | H | H | Cl | A26 | J2 | Q152 |
| 4-4111 | H | H | Br | A26 | J2 | Q152 |
| 4-4112 | H | H | CH₂F | A26 | J2 | Q152 |
| 4-4113 | H | H | CHF2 | A26 | J2 | Q152 |
| 4-4114 | H | H | Et | A26 | J2 | Q152 |
| 4-4115 | H | H | Pr | A26 | J2 | Q152 |
| 4-4116 | H | H | cPr | A26 | J2 | Q152 |
| 4-4117 | H | H | ethynyl | A26 | J2 | Q152 |
| 4-4118 | F | H | Me | A26 | J2 | Q152 |
| 4-4119 | F | F | Me | A26 | J2 | Q152 |
| 4-4120 | Me | H | Me | A26 | J2 | Q152 |
| 4-4121 | Me | Me | Me | A26 | J2 | Q152 |
| 4-4122 | H | H | H | A27 | J2 | Q152 |
| 4-4123 | H | H | Cl | A27 | J2 | Q152 |
| 4-4124 | H | H | Br | A27 | J2 | Q152 |
| 4-4125 | H | H | CH₂F | A27 | J2 | Q152 |
| 4-4126 | H | H | CHF2 | A27 | J2 | Q152 |
| 4-4127 | H | H | Et | A27 | J2 | Q152 |
| 4-4128 | H | H | Pr | A27 | J2 | Q152 |
| 4-4129 | H | H | cPr | A27 | J2 | Q152 |
| 4-4130 | H | H | ethynyl | A27 | J2 | Q152 |
| 4-4131 | F | H | Me | A27 | J2 | Q152 |
| 4-4132 | F | F | Me | A27 | J2 | Q152 |
| 4-4133 | Me | H | Me | A27 | J2 | Q152 |
| 4-4134 | Me | Me | Me | A27 | J2 | Q152 |
| 4-4135 | H | H | H | A28 | J2 | Q152 |
| 4-4136 | H | H | Cl | A28 | J2 | Q152 |
| 4-4137 | H | H | Br | A28 | J2 | Q152 |
| 4-4138 | H | H | CH₂F | A28 | J2 | Q152 |
| 4-4139 | H | H | CHF2 | A28 | J2 | Q152 |
| 4-4140 | H | H | Et | A28 | J2 | Q152 |
| 4-4141 | H | H | Pr | A28 | J2 | Q152 |
| 4-4142 | H | H | cPr | A28 | J2 | Q152 |
| 4-4143 | H | H | ethynyl | A28 | J2 | Q152 |
| 4-4144 | F | H | Me | A28 | J2 | Q152 |
| 4-4145 | F | F | Me | A28 | J2 | Q152 |
| 4-4146 | Me | H | Me | A28 | J2 | Q152 |
| 4-4147 | Me | Me | Me | A28 | J2 | Q152 |
| 4-4148 | H | H | H | A29 | J2 | Q152 |
| 4-4149 | H | H | Cl | A29 | J2 | Q152 |
| 4-4150 | H | H | Br | A29 | J2 | Q152 |
| 4-4151 | H | H | CH₂F | A29 | J2 | Q152 |
| 4-4152 | H | H | CHF2 | A29 | J2 | Q152 |
| 4-4153 | H | H | Et | A29 | J2 | Q152 |
| 4-4154 | H | H | Pr | A29 | J2 | Q152 |
| 4-4155 | H | H | cPr | A29 | J2 | Q152 |
| 4-4156 | H | H | ethynyl | A29 | J2 | Q152 |
| 4-4157 | F | H | Me | A29 | J2 | Q152 |
| 4-4158 | F | F | Me | A29 | J2 | Q152 |
| 4-4159 | Me | H | Me | A29 | J2 | Q152 |
| 4-4160 | Me | Me | Me | A29 | J2 | Q152 |
| 4-4161 | H | H | H | A30 | J2 | Q152 |
| 4-4162 | H | H | Cl | A30 | J2 | Q152 |
| 4-4163 | H | H | Br | A30 | J2 | Q152 |
| 4-4164 | H | H | CH₂F | A30 | J2 | Q152 |
| 4-4165 | H | H | CHF2 | A30 | J2 | Q152 |
| 4-4166 | H | H | Et | A30 | J2 | Q152 |
| 4-4167 | H | H | Pr | A30 | J2 | Q152 |
| 4-4168 | H | H | cPr | A30 | J2 | Q152 |
| 4-4169 | H | H | ethynyl | A30 | J2 | Q152 |
| 4-4170 | F | H | Me | A30 | J2 | Q152 |
| 4-4171 | F | F | Me | A30 | J2 | Q152 |
| 4-4172 | Me | H | Me | A30 | J2 | Q152 |
| 4-4173 | Me | Me | Me | A30 | J2 | Q152 |
| 4-4174 | H | H | H | A31 | J2 | Q152 |
| 4-4175 | H | H | Cl | A31 | J2 | Q152 |
| 4-4176 | H | H | Br | A31 | J2 | Q152 |
| 4-4177 | H | H | CH₂F | A31 | J2 | Q152 |
| 4-4178 | H | H | CHF2 | A31 | J2 | Q152 |
| 4-4179 | H | H | Et | A31 | J2 | Q152 |
| 4-4180 | H | H | Pr | A31 | J2 | Q152 |
| 4-4181 | H | H | cPr | A31 | J2 | Q152 |
| 4-4182 | H | H | ethynyl | A31 | J2 | Q152 |
| 4-4183 | F | H | Me | A31 | J2 | Q152 |
| 4-4184 | F | F | Me | A31 | J2 | Q152 |
| 4-4185 | Me | H | Me | A31 | J2 | Q152 |
| 4-4186 | Me | Me | Me | A31 | J2 | Q152 |
| 4-4187 | H | H | H | A51 | J2 | Q152 |
| 4-4188 | H | H | Cl | A51 | J2 | Q152 |
| 4-4189 | H | H | Br | A51 | J2 | Q152 |
| 4-4190 | H | H | CH₂F | A51 | J2 | Q152 |
| 4-4191 | H | H | CHF2 | A51 | J2 | Q152 |
| 4-4192 | H | H | Et | A51 | J2 | Q152 |
| 4-4193 | H | H | Pr | A51 | J2 | Q152 |
| 4-4194 | H | H | cPr | A51 | J2 | Q152 |
| 4-4195 | H | H | ethynyl | A51 | J2 | Q152 |
| 4-4196 | F | H | Me | A51 | J2 | Q152 |
| 4-4197 | F | F | Me | A51 | J2 | Q152 |
| 4-4198 | Me | H | Me | A51 | J2 | Q152 |
| 4-4199 | Me | Me | Me | A51 | J2 | Q152 |
| 4-4200 | H | H | H | A1 | J1 | Q152 |
| 4-4201 | H | H | Cl | A1 | J1 | Q152 |
| 4-4202 | H | H | Br | A1 | J1 | Q152 |
| 4-4203 | H | H | CH₂F | A1 | J1 | Q152 |
| 4-4204 | H | H | CHF2 | A1 | J1 | Q152 |
| 4-4205 | H | H | Et | A1 | J1 | Q152 |
| 4-4206 | H | H | Pr | A1 | J1 | Q152 |
| 4-4207 | H | H | cPr | A1 | J1 | Q152 |
| 4-4208 | H | H | ethynyl | A1 | J1 | Q152 |
| 4-4209 | F | H | Me | A1 | J1 | Q152 |
| 4-4210 | F | F | Me | A1 | J1 | Q152 |
| 4-4211 | Me | H | Me | A1 | J1 | Q152 |
| 4-4212 | Me | Me | Me | A1 | J1 | Q152 |
| 4-4213 | H | H | H | A2 | J1 | Q152 |
| 4-4214 | H | H | Cl | A2 | J1 | Q152 |
| 4-4215 | H | H | Br | A2 | J1 | Q152 |
| 4-4216 | H | H | CH₂F | A2 | J1 | Q152 |
| 4-4217 | H | H | CHF2 | A2 | J1 | Q152 |
| 4-4218 | H | H | Et | A2 | J1 | Q152 |
| 4-4219 | H | H | Pr | A2 | J1 | Q152 |
| 4-4220 | H | H | cPr | A2 | J1 | Q152 |
| 4-4221 | H | H | ethynyl | A2 | J1 | Q152 |
| 4-4222 | F | H | Me | A2 | J1 | Q152 |
| 4-4223 | F | F | Me | A2 | J1 | Q152 |
| 4-4224 | Me | H | Me | A2 | J1 | Q152 |
| 4-4225 | Me | Me | Me | A2 | J1 | Q152 |
| 4-4226 | H | H | H | A6 | J1 | Q152 |
| 4-4227 | H | H | Cl | A6 | J1 | Q152 |
| 4-4228 | H | H | Br | A6 | J1 | Q152 |
| 4-4229 | H | H | CH₂F | A6 | J1 | Q152 |
| 4-4230 | H | H | CHF2 | A6 | J1 | Q152 |
| 4-4231 | H | H | Et | A6 | J1 | Q152 |
| 4-4232 | H | H | Pr | A6 | J1 | Q152 |
| 4-4233 | H | H | cPr | A6 | J1 | Q152 |
| 4-4234 | H | H | ethynyl | A6 | J1 | Q152 |
| 4-4235 | F | H | Me | A6 | J1 | Q152 |
| 4-4236 | F | F | Me | A6 | J1 | Q152 |
| 4-4237 | Me | H | Me | A6 | J1 | Q152 |
| 4-4238 | Me | Me | Me | A6 | J1 | Q152 |
| 4-4239 | H | H | H | A9 | J1 | Q152 |
| 4-4240 | H | H | Cl | A9 | J1 | Q152 |
| 4-4241 | H | H | Br | A9 | J1 | Q152 |
| 4-4242 | H | H | CH₂F | A9 | J1 | Q152 |
| 4-4243 | H | H | CHF2 | A9 | J1 | Q152 |
| 4-4244 | H | H | Et | A9 | J1 | Q152 |
| 4-4245 | H | H | Pr | A9 | J1 | Q152 |
| 4-4246 | H | H | cPr | A9 | J1 | Q152 |
| 4-4247 | H | H | ethynyl | A9 | J1 | Q152 |
| 4-4248 | F | H | Me | A9 | J1 | Q152 |
| 4-4249 | F | F | Me | A9 | J1 | Q152 |
| 4-4250 | Me | H | Me | A9 | J1 | Q152 |
| 4-4251 | Me | Me | Me | A9 | J1 | Q152 |
| 4-4252 | H | H | H | A11 | J1 | Q152 |
| 4-4253 | H | H | Cl | A11 | J1 | Q152 |
| 4-4254 | H | H | Br | A11 | J1 | Q152 |
| 4-4255 | H | H | CH₂F | A11 | J1 | Q152 |
| 4-4256 | H | H | CHF2 | A11 | J1 | Q152 |
| 4-4257 | H | H | Et | A11 | J1 | Q152 |
| 4-4258 | H | H | Pr | A11 | J1 | Q152 |
| 4-4259 | H | H | cPr | A11 | J1 | Q152 |
| 4-4260 | H | H | ethynyl | A11 | J1 | Q152 |
| 4-4261 | F | H | Me | A11 | J1 | Q152 |
| 4-4262 | F | F | Me | A11 | J1 | Q152 |
| 4-4263 | Me | H | Me | A11 | J1 | Q152 |
| 4-4264 | Me | Me | Me | A11 | J1 | Q152 |
| 4-4265 | H | H | H | A12 | J1 | Q152 |
| 4-4266 | H | H | Cl | A12 | J1 | Q152 |
| 4-4267 | H | H | Br | A12 | J1 | Q152 |
| 4-4268 | H | H | CH₂F | A12 | J1 | Q152 |
| 4-4269 | H | H | CHF2 | A12 | J1 | Q152 |
| 4-4270 | H | H | Et | A12 | J1 | Q152 |
| 4-4271 | H | H | Pr | A12 | J1 | Q152 |
| 4-4272 | H | H | cPr | A12 | J1 | Q152 |
| 4-4273 | H | H | ethynyl | A12 | J1 | Q152 |
| 4-4274 | F | H | Me | A12 | J1 | Q152 |
| 4-4275 | F | F | Me | A12 | J1 | Q152 |
| 4-4276 | Me | H | Me | A12 | J1 | Q152 |
| 4-4277 | Me | Me | Me | A12 | J1 | Q152 |
| 4-4278 | H | H | H | A19 | J1 | Q152 |
| 4-4279 | H | H | Cl | A19 | J1 | Q152 |
| 4-4280 | H | H | Br | A19 | J1 | Q152 |
| 4-4281 | H | H | CH₂F | A19 | J1 | Q152 |
| 4-4282 | H | H | CHF2 | A19 | J1 | Q152 |
| 4-4283 | H | H | Et | A19 | J1 | Q152 |
| 4-4284 | H | H | Pr | A19 | J1 | Q152 |
| 4-4285 | H | H | cPr | A19 | J1 | Q152 |
| 4-4286 | H | H | ethynyl | A19 | J1 | Q152 |
| 4-4287 | F | H | Me | A19 | J1 | Q152 |
| 4-4288 | F | F | Me | A19 | J1 | Q152 |
| 4-4289 | Me | H | Me | A19 | J1 | Q152 |
| 4-4290 | Me | Me | Me | A19 | J1 | Q152 |
| 4-4291 | H | H | H | A20 | J1 | Q152 |
| 4-4292 | H | H | Cl | A20 | J1 | Q152 |
| 4-4293 | H | H | Br | A20 | J1 | Q152 |
| 4-4294 | H | H | CH₂F | A20 | J1 | Q152 |
| 4-4295 | H | H | CHF2 | A20 | J1 | Q152 |
| 4-4296 | H | H | Et | A20 | J1 | Q152 |
| 4-4297 | H | H | Pr | A20 | J1 | Q152 |
| 4-4298 | H | H | cPr | A20 | J1 | Q152 |
| 4-4299 | H | H | ethynyl | A20 | J1 | Q152 |
| 4-4300 | F | H | Me | A20 | J1 | Q152 |
| 4-4301 | F | F | Me | A20 | J1 | Q152 |
| 4-4302 | Me | H | Me | A20 | J1 | Q152 |
| 4-4303 | Me | Me | Me | A20 | J1 | Q152 |
| 4-4304 | H | H | H | A21 | J1 | Q152 |
| 4-4305 | H | H | Cl | A21 | J1 | Q152 |
| 4-4306 | H | H | Br | A21 | J1 | Q152 |
| 4-4307 | H | H | CH₂F | A21 | J1 | Q152 |
| 4-4308 | H | H | CHF2 | A21 | J1 | Q152 |
| 4-4309 | H | H | Et | A21 | J1 | Q152 |
| 4-4310 | H | H | Pr | A21 | J1 | Q152 |
| 4-4311 | H | H | cPr | A21 | J1 | Q152 |
| 4-4312 | H | H | ethynyl | A21 | J1 | Q152 |
| 4-4313 | F | H | Me | A21 | J1 | Q152 |
| 4-4314 | F | F | Me | A21 | J1 | Q152 |
| 4-4315 | Me | H | Me | A21 | J1 | Q152 |
| 4-4316 | Me | Me | Me | A21 | J1 | Q152 |
| 4-4317 | H | H | H | A22 | J1 | Q152 |
| 4-4318 | H | H | Cl | A22 | J1 | Q152 |
| 4-4319 | H | H | Br | A22 | J1 | Q152 |
| 4-4320 | H | H | CH₂F | A22 | J1 | Q152 |
| 4-4321 | H | H | CHF2 | A22 | J1 | Q152 |
| 4-4322 | H | H | Et | A22 | J1 | Q152 |
| 4-4323 | H | H | Pr | A22 | J1 | Q152 |
| 4-4324 | H | H | cPr | A22 | J1 | Q152 |
| 4-4325 | H | H | ethynyl | A22 | J1 | Q152 |
| 4-4326 | F | H | Me | A22 | J1 | Q152 |
| 4-4327 | F | F | Me | A22 | J1 | Q152 |
| 4-4328 | Me | H | Me | A22 | J1 | Q152 |
| 4-4329 | Me | Me | Me | A22 | J1 | Q152 |
| 4-4330 | H | H | H | A26 | J1 | Q152 |
| 4-4331 | H | H | Cl | A26 | J1 | Q152 |
| 4-4332 | H | H | Br | A26 | J1 | Q152 |
| 4-4333 | H | H | CH₂F | A26 | J1 | Q152 |
| 4-4334 | H | H | CHF2 | A26 | J1 | Q152 |
| 4-4335 | H | H | Et | A26 | J1 | Q152 |
| 4-4336 | H | H | Pr | A26 | J1 | Q152 |
| 4-4337 | H | H | cPr | A26 | J1 | Q152 |
| 4-4338 | H | H | ethynyl | A26 | J1 | Q152 |
| 4-4339 | F | H | Me | A26 | J1 | Q152 |
| 4-4340 | F | F | Me | A26 | J1 | Q152 |
| 4-4341 | Me | H | Me | A26 | J1 | Q152 |
| 4-4342 | Me | Me | Me | A26 | J1 | Q152 |
| 4-4343 | H | H | H | A27 | J1 | Q152 |
| 4-4344 | H | H | Cl | A27 | J1 | Q152 |
| 4-4345 | H | H | Br | A27 | J1 | Q152 |
| 4-4346 | H | H | CH₂F | A27 | J1 | Q152 |
| 4-4347 | H | H | CHF2 | A27 | J1 | Q152 |
| 4-4348 | H | H | Et | A27 | J1 | Q152 |
| 4-4349 | H | H | Pr | A27 | J1 | Q152 |
| 4-4350 | H | H | cPr | A27 | J1 | Q152 |
| 4-4351 | H | H | ethynyl | A27 | J1 | Q152 |
| 4-4352 | F | H | Me | A27 | J1 | Q152 |
| 4-4353 | F | F | Me | A27 | J1 | Q152 |
| 4-4354 | Me | H | Me | A27 | J1 | Q152 |
| 4-4355 | Me | Me | Me | A27 | J1 | Q152 |
| 4-4356 | H | H | H | A28 | J1 | Q152 |
| 4-4357 | H | H | Cl | A28 | J1 | Q152 |
| 4-4358 | H | H | Br | A28 | J1 | Q152 |
| 4-4359 | H | H | CH₂F | A28 | J1 | Q152 |
| 4-4360 | H | H | CHF2 | A28 | J1 | Q152 |
| 4-4361 | H | H | Et | A28 | J1 | Q152 |
| 4-4362 | H | H | Pr | A28 | J1 | Q152 |
| 4-4363 | H | H | cPr | A28 | J1 | Q152 |
| 4-4364 | H | H | ethynyl | A28 | J1 | Q152 |
| 4-4365 | F | H | Me | A28 | J1 | Q152 |
| 4-4366 | F | F | Me | A28 | J1 | Q152 |
| 4-4367 | Me | H | Me | A28 | J1 | Q152 |
| 4-4368 | Me | Me | Me | A28 | J1 | Q152 |
| 4-4369 | H | H | H | A29 | J1 | Q152 |
| 4-4370 | H | H | Cl | A29 | J1 | Q152 |
| 4-4371 | H | H | Br | A29 | J1 | Q152 |
| 4-4372 | H | H | CH₂F | A29 | J1 | Q152 |
| 4-4373 | H | H | CHF2 | A29 | J1 | Q152 |
| 4-4374 | H | H | Et | A29 | J1 | Q152 |
| 4-4375 | H | H | Pr | A29 | J1 | Q152 |
| 4-4376 | H | H | cPr | A29 | J1 | Q152 |
| 4-4377 | H | H | ethynyl | A29 | J1 | Q152 |
| 4-4378 | F | H | Me | A29 | J1 | Q152 |
| 4-4379 | F | F | Me | A29 | J1 | Q152 |
| 4-4380 | Me | H | Me | A29 | J1 | Q152 |
| 4-4381 | Me | Me | Me | A29 | J1 | Q152 |
| 4-4382 | H | H | H | A30 | J1 | Q152 |
| 4-4383 | H | H | Cl | A30 | J1 | Q152 |
| 4-4384 | H | H | Br | A30 | J1 | Q152 |
| 4-4385 | H | H | CH₂F | A30 | J1 | Q152 |
| 4-4386 | H | H | CHF2 | A30 | J1 | Q152 |
| 4-4387 | H | H | Et | A30 | J1 | Q152 |
| 4-4388 | H | H | Pr | A30 | J1 | Q152 |
| 4-4389 | H | H | cPr | A30 | J1 | Q152 |
| 4-4390 | H | H | ethynyl | A30 | J1 | Q152 |
| 4-4391 | F | H | Me | A30 | J1 | Q152 |
| 4-4392 | F | F | Me | A30 | J1 | Q152 |
| 4-4393 | Me | H | Me | A30 | J1 | Q152 |
| 4-4394 | Me | Me | Me | A30 | J1 | Q152 |
| 4-4395 | H | H | H | A31 | J1 | Q152 |
| 4-4396 | H | H | Cl | A31 | J1 | Q152 |
| 4-4397 | H | H | Br | A31 | J1 | Q152 |
| 4-4398 | H | H | CH₂F | A31 | J1 | Q152 |
| 4-4399 | H | H | CHF2 | A31 | J1 | Q152 |
| 4-4400 | H | H | Et | A31 | J1 | Q152 |
| 4-4401 | H | H | Pr | A31 | J1 | Q152 |
| 4-4402 | H | H | cPr | A31 | J1 | Q152 |
| 4-4403 | H | H | ethynyl | A31 | J1 | Q152 |
| 4-4404 | F | H | Me | A31 | J1 | Q152 |
| 4-4405 | F | F | Me | A31 | J1 | Q152 |
| 4-4406 | Me | H | Me | A31 | J1 | Q152 |
| 4-4407 | Me | Me | Me | A31 | J1 | Q152 |
| 4-4408 | H | H | H | A51 | J1 | Q152 |
| 4-4409 | H | H | Cl | A51 | J1 | Q152 |
| 4-4410 | H | H | Br | A51 | J1 | Q152 |
| 4-4411 | H | H | CH₂F | A51 | J1 | Q152 |
| 4-4412 | H | H | CHF2 | A51 | J1 | Q152 |
| 4-4413 | H | H | Et | A51 | J1 | Q152 |
| 4-4414 | H | H | Pr | A51 | J1 | Q152 |
| 4-4415 | H | H | cPr | A51 | J1 | Q152 |
| 4-4416 | H | H | ethynyl | A51 | J1 | Q152 |
| 4-4417 | F | H | Me | A51 | J1 | Q152 |
| 4-4418 | F | F | Me | A51 | J1 | Q152 |
| 4-4419 | Me | H | Me | A51 | J1 | Q152 |
| 4-4420 | Me | Me | Me | A51 | J1 | Q152 |
| 4-4421 | H | H | H | A1 | J2 | Q154 |
| 4-4422 | H | H | Cl | A1 | J2 | Q154 |
| 4-4423 | H | H | Br | A1 | J2 | Q154 |
| 4-4424 | H | H | CH₂F | A1 | J2 | Q154 |
| 4-4425 | H | H | CHF2 | A1 | J2 | Q154 |
| 4-4426 | H | H | Et | A1 | J2 | Q154 |
| 4-4427 | H | H | Pr | A1 | J2 | Q154 |
| 4-4428 | H | H | cPr | A1 | J2 | Q154 |
| 4-4429 | H | H | ethynyl | A1 | J2 | Q154 |
| 4-4430 | F | H | Me | A1 | J2 | Q154 |
| 4-4431 | F | F | Me | A1 | J2 | Q154 |
| 4-4432 | Me | H | Me | A1 | J2 | Q154 |
| 4-4433 | Me | Me | Me | A1 | J2 | Q154 |
| 4-4434 | H | H | H | A2 | J2 | Q154 |
| 4-4435 | H | H | Cl | A2 | J2 | Q154 |
| 4-4436 | H | H | Br | A2 | J2 | Q154 |
| 4-4437 | H | H | CH₂F | A2 | J2 | Q154 |
| 4-4438 | H | H | CHF2 | A2 | J2 | Q154 |
| 4-4439 | H | H | Et | A2 | J2 | Q154 |
| 4-4440 | H | H | Pr | A2 | J2 | Q154 |
| 4-4441 | H | H | cPr | A2 | J2 | Q154 |
| 4-4442 | H | H | ethynyl | A2 | J2 | Q154 |
| 4-4443 | F | H | Me | A2 | J2 | Q154 |
| 4-4444 | F | F | Me | A2 | J2 | Q154 |
| 4-4445 | Me | H | Me | A2 | J2 | Q154 |
| 4-4446 | Me | Me | Me | A2 | J2 | Q154 |
| 4-4447 | H | H | H | A6 | J2 | Q154 |
| 4-4448 | H | H | Cl | A6 | J2 | Q154 |
| 4-4449 | H | H | Br | A6 | J2 | Q154 |
| 4-4450 | H | H | CH₂F | A6 | J2 | Q154 |
| 4-4451 | H | H | CHF2 | A6 | J2 | Q154 |
| 4-4452 | H | H | Et | A6 | J2 | Q154 |
| 4-4453 | H | H | Pr | A6 | J2 | Q154 |
| 4-4454 | H | H | cPr | A6 | J2 | Q154 |
| 4-4455 | H | H | ethynyl | A6 | J2 | Q154 |
| 4-4456 | F | H | Me | A6 | J2 | Q154 |
| 4-4457 | F | F | Me | A6 | J2 | Q154 |
| 4-4458 | Me | H | Me | A6 | J2 | Q154 |
| 4-4459 | Me | Me | Me | A6 | J2 | Q154 |
| 4-4460 | H | H | H | A9 | J2 | Q154 |
| 4-4461 | H | H | Cl | A9 | J2 | Q154 |
| 4-4462 | H | H | Br | A9 | J2 | Q154 |
| 4-4463 | H | H | CH₂F | A9 | J2 | Q154 |
| 4-4464 | H | H | CHF2 | A9 | J2 | Q154 |
| 4-4465 | H | H | Et | A9 | J2 | Q154 |
| 4-4466 | H | H | Pr | A9 | J2 | Q154 |
| 4-4467 | H | H | cPr | A9 | J2 | Q154 |
| 4-4468 | H | H | ethynyl | A9 | J2 | Q154 |
| 4-4469 | F | H | Me | A9 | J2 | Q154 |
| 4-4470 | F | F | Me | A9 | J2 | Q154 |
| 4-4471 | Me | H | Me | A9 | J2 | Q154 |
| 4-4472 | Me | Me | Me | A9 | J2 | Q154 |
| 4-4473 | H | H | H | A11 | J2 | Q154 |
| 4-4474 | H | H | Cl | A11 | J2 | Q154 |
| 4-4475 | H | H | Br | A11 | J2 | Q154 |
| 4-4476 | H | H | CH₂F | A11 | J2 | Q154 |
| 4-4477 | H | H | CHF2 | A11 | J2 | Q154 |
| 4-4478 | H | H | Et | A11 | J2 | Q154 |
| 4-4479 | H | H | Pr | A11 | J2 | Q154 |
| 4-4480 | H | H | cPr | A11 | J2 | Q154 |
| 4-4481 | H | H | ethynyl | A11 | J2 | Q154 |
| 4-4482 | F | H | Me | A11 | J2 | Q154 |
| 4-4483 | F | F | Me | A11 | J2 | Q154 |
| 4-4484 | Me | H | Me | A11 | J2 | Q154 |
| 4-4485 | Me | Me | Me | A11 | J2 | Q154 |
| 4-4486 | H | H | H | A12 | J2 | Q154 |
| 4-4487 | H | H | Cl | A12 | J2 | Q154 |
| 4-4488 | H | H | Br | A12 | J2 | Q154 |
| 4-4489 | H | H | CH₂F | A12 | J2 | Q154 |
| 4-4490 | H | H | CHF2 | A12 | J2 | Q154 |
| 4-4491 | H | H | Et | A12 | J2 | Q154 |
| 4-4492 | H | H | Pr | A12 | J2 | Q154 |
| 4-4493 | H | H | cPr | A12 | J2 | Q154 |
| 4-4494 | H | H | ethynyl | A12 | J2 | Q154 |
| 4-4495 | F | H | Me | A12 | J2 | Q154 |
| 4-4496 | F | F | Me | A12 | J2 | Q154 |
| 4-4497 | Me | H | Me | A12 | J2 | Q154 |
| 4-4498 | Me | Me | Me | A12 | J2 | Q154 |
| 4-4499 | H | H | H | A19 | J2 | Q154 |
| 4-4500 | H | H | Cl | A19 | J2 | Q154 |
| 4-4501 | H | H | Br | A19 | J2 | Q154 |
| 4-4502 | H | H | CH₂F | A19 | J2 | Q154 |
| 4-4503 | H | H | CHF2 | A19 | J2 | Q154 |
| 4-4504 | H | H | Et | A19 | J2 | Q154 |
| 4-4505 | H | H | Pr | A19 | J2 | Q154 |
| 4-4506 | H | H | cPr | A19 | J2 | Q154 |
| 4-4507 | H | H | ethynyl | A19 | J2 | Q154 |
| 4-4508 | F | H | Me | A19 | J2 | Q154 |
| 4-4509 | F | F | Me | A19 | J2 | Q154 |
| 4-4510 | Me | H | Me | A19 | J2 | Q154 |
| 4-4511 | Me | Me | Me | A19 | J2 | Q154 |
| 4-4512 | H | H | H | A20 | J2 | Q154 |
| 4-4513 | H | H | Cl | A20 | J2 | Q154 |
| 4-4514 | H | H | Br | A20 | J2 | Q154 |
| 4-4515 | H | H | CH₂F | A20 | J2 | Q154 |
| 4-4516 | H | H | CHF2 | A20 | J2 | Q154 |
| 4-4517 | H | H | Et | A20 | J2 | Q154 |
| 4-4518 | H | H | Pr | A20 | J2 | Q154 |
| 4-4519 | H | H | cPr | A20 | J2 | Q154 |
| 4-4520 | H | H | ethynyl | A20 | J2 | Q154 |
| 4-4521 | F | H | Me | A20 | J2 | Q154 |
| 4-4522 | F | F | Me | A20 | J2 | Q154 |
| 4-4523 | Me | H | Me | A20 | J2 | Q154 |
| 4-4524 | Me | Me | Me | A20 | J2 | Q154 |
| 4-4525 | H | H | H | A21 | J2 | Q154 |
| 4-4526 | H | H | Cl | A21 | J2 | Q154 |
| 4-4527 | H | H | Br | A21 | J2 | Q154 |
| 4-4528 | H | H | CH₂F | A21 | J2 | Q154 |
| 4-4529 | H | H | CHF2 | A21 | J2 | Q154 |
| 4-4530 | H | H | Et | A21 | J2 | Q154 |
| 4-4531 | H | H | Pr | A21 | J2 | Q154 |
| 4-4532 | H | H | cPr | A21 | J2 | Q154 |
| 4-4533 | H | H | ethynyl | A21 | J2 | Q154 |
| 4-4534 | F | H | Me | A21 | J2 | Q154 |
| 4-4535 | F | F | Me | A21 | J2 | Q154 |
| 4-4536 | Me | H | Me | A21 | J2 | Q154 |
| 4-4537 | Me | Me | Me | A21 | J2 | Q154 |
| 4-4538 | H | H | H | A22 | J2 | Q154 |
| 4-4539 | H | H | Cl | A22 | J2 | Q154 |
| 4-4540 | H | H | Br | A22 | J2 | Q154 |
| 4-4541 | H | H | CH₂F | A22 | J2 | Q154 |
| 4-4542 | H | H | CHF2 | A22 | J2 | Q154 |
| 4-4543 | H | H | Et | A22 | J2 | Q154 |
| 4-4544 | H | H | Pr | A22 | J2 | Q154 |
| 4-4545 | H | H | cPr | A22 | J2 | Q154 |
| 4-4546 | H | H | ethynyl | A22 | J2 | Q154 |
| 4-4547 | F | H | Me | A22 | J2 | Q154 |
| 4-4548 | F | F | Me | A22 | J2 | Q154 |
| 4-4549 | Me | H | Me | A22 | J2 | Q154 |
| 4-4550 | Me | Me | Me | A22 | J2 | Q154 |
| 4-4551 | H | H | H | A26 | J2 | Q154 |
| 4-4552 | H | H | Cl | A26 | J2 | Q154 |
| 4-4553 | H | H | Br | A26 | J2 | Q154 |
| 4-4554 | H | H | CH₂F | A26 | J2 | Q154 |
| 4-4555 | H | H | CHF2 | A26 | J2 | Q154 |
| 4-4556 | H | H | Et | A26 | J2 | Q154 |
| 4-4557 | H | H | Pr | A26 | J2 | Q154 |
| 4-4558 | H | H | cPr | A26 | J2 | Q154 |
| 4-4559 | H | H | ethynyl | A26 | J2 | Q154 |
| 4-4560 | F | H | Me | A26 | J2 | Q154 |
| 4-4561 | F | F | Me | A26 | J2 | Q154 |
| 4-4562 | Me | H | Me | A26 | J2 | Q154 |
| 4-4563 | Me | Me | Me | A26 | J2 | Q154 |
| 4-4564 | H | H | H | A27 | J2 | Q154 |
| 4-4565 | H | H | Cl | A27 | J2 | Q154 |
| 4-4566 | H | H | Br | A27 | J2 | Q154 |
| 4-4567 | H | H | CH₂F | A27 | J2 | Q154 |
| 4-4568 | H | H | CHF2 | A27 | J2 | Q154 |
| 4-4569 | H | H | Et | A27 | J2 | Q154 |
| 4-4570 | H | H | Pr | A27 | J2 | Q154 |
| 4-4571 | H | H | cPr | A27 | J2 | Q154 |
| 4-4572 | H | H | ethynyl | A27 | J2 | Q154 |
| 4-4573 | F | H | Me | A27 | J2 | Q154 |
| 4-4574 | F | F | Me | A27 | J2 | Q154 |
| 4-4575 | Me | H | Me | A27 | J2 | Q154 |
| 4-4576 | Me | Me | Me | A27 | J2 | Q154 |
| 4-4577 | H | H | H | A28 | J2 | Q154 |
| 4-4578 | H | H | Cl | A28 | J2 | Q154 |
| 4-4579 | H | H | Br | A28 | J2 | Q154 |
| 4-4580 | H | H | CH₂F | A28 | J2 | Q154 |
| 4-4581 | H | H | CHF2 | A28 | J2 | Q154 |
| 4-4582 | H | H | Et | A28 | J2 | Q154 |
| 4-4583 | H | H | Pr | A28 | J2 | Q154 |
| 4-4584 | H | H | cPr | A28 | J2 | Q154 |
| 4-4585 | H | H | ethynyl | A28 | J2 | Q154 |
| 4-4586 | F | H | Me | A28 | J2 | Q154 |
| 4-4587 | F | F | Me | A28 | J2 | Q154 |
| 4-4588 | Me | H | Me | A28 | J2 | Q154 |
| 4-4589 | Me | Me | Me | A28 | J2 | Q154 |
| 4-4590 | H | H | H | A29 | J2 | Q154 |
| 4-4591 | H | H | Cl | A29 | J2 | Q154 |
| 4-4592 | H | H | Br | A29 | J2 | Q154 |
| 4-4593 | H | H | CH₂F | A29 | J2 | Q154 |
| 4-4594 | H | H | CHF2 | A29 | J2 | Q154 |
| 4-4595 | H | H | Et | A29 | J2 | Q154 |
| 4-4596 | H | H | Pr | A29 | J2 | Q154 |
| 4-4597 | H | H | cPr | A29 | J2 | Q154 |
| 4-4598 | H | H | ethynyl | A29 | J2 | Q154 |
| 4-4599 | F | H | Me | A29 | J2 | Q154 |
| 4-4600 | F | F | Me | A29 | J2 | Q154 |
| 4-4601 | Me | H | Me | A29 | J2 | Q154 |
| 4-4602 | Me | Me | Me | A29 | J2 | Q154 |
| 4-4603 | H | H | H | A30 | J2 | Q154 |
| 4-4604 | H | H | Cl | A30 | J2 | Q154 |
| 4-4605 | H | H | Br | A30 | J2 | Q154 |
| 4-4606 | H | H | CH₂F | A30 | J2 | Q154 |
| 4-4607 | H | H | CHF2 | A30 | J2 | Q154 |
| 4-4608 | H | H | Et | A30 | J2 | Q154 |
| 4-4609 | H | H | Pr | A30 | J2 | Q154 |
| 4-4610 | H | H | cPr | A30 | J2 | Q154 |
| 4-4611 | H | H | ethynyl | A30 | J2 | Q154 |
| 4-4612 | F | H | Me | A30 | J2 | Q154 |
| 4-4613 | F | F | Me | A30 | J2 | Q154 |
| 4-4614 | Me | H | Me | A30 | J2 | Q154 |
| 4-4615 | Me | Me | Me | A30 | J2 | Q154 |
| 4-4616 | H | H | H | A31 | J2 | Q154 |
| 4-4617 | H | H | Cl | A31 | J2 | Q154 |
| 4-4618 | H | H | Br | A31 | J2 | Q154 |
| 4-4619 | H | H | CH₂F | A31 | J2 | Q154 |
| 4-4620 | H | H | CHF2 | A31 | J2 | Q154 |
| 4-4621 | H | H | Et | A31 | J2 | Q154 |
| 4-4622 | H | H | Pr | A31 | J2 | Q154 |
| 4-4623 | H | H | cPr | A31 | J2 | Q154 |
| 4-4624 | H | H | ethynyl | A31 | J2 | Q154 |
| 4-4625 | F | H | Me | A31 | J2 | Q154 |
| 4-4626 | F | F | Me | A31 | J2 | Q154 |
| 4-4627 | Me | H | Me | A31 | J2 | Q154 |
| 4-4628 | Me | Me | Me | A31 | J2 | Q154 |
| 4-4629 | H | H | H | A51 | J2 | Q154 |
| 4-4630 | H | H | Cl | A51 | J2 | Q154 |
| 4-4631 | H | H | Br | A51 | J2 | Q154 |
| 4-4632 | H | H | CH₂F | A51 | J2 | Q154 |
| 4-4633 | H | H | CHF2 | A51 | J2 | Q154 |
| 4-4634 | H | H | Et | A51 | J2 | Q154 |
| 4-4635 | H | H | Pr | A51 | J2 | Q154 |
| 4-4636 | H | H | cPr | A51 | J2 | Q154 |
| 4-4637 | H | H | ethynyl | A51 | J2 | Q154 |
| 4-4638 | F | H | Me | A51 | J2 | Q154 |
| 4-4639 | F | F | Me | A51 | J2 | Q154 |
| 4-4640 | Me | H | Me | A51 | J2 | Q154 |
| 4-4641 | Me | Me | Me | A51 | J2 | Q154 |
| 4-4642 | H | H | H | A1 | J1 | Q154 |
| 4-4643 | H | H | Cl | A1 | J1 | Q154 |
| 4-4644 | H | H | Br | A1 | J1 | Q154 |
| 4-4645 | H | H | CH₂F | A1 | J1 | Q154 |
| 4-4646 | H | H | CHF2 | A1 | J1 | Q154 |
| 4-4647 | H | H | Et | A1 | J1 | Q154 |
| 4-4648 | H | H | Pr | A1 | J1 | Q154 |
| 4-4649 | H | H | cPr | A1 | J1 | Q154 |
| 4-4650 | H | H | ethynyl | A1 | J1 | Q154 |
| 4-4651 | F | H | Me | A1 | J1 | Q154 |
| 4-4652 | F | F | Me | A1 | J1 | Q154 |
| 4-4653 | Me | H | Me | A1 | J1 | Q154 |
| 4-4654 | Me | Me | Me | A1 | J1 | Q154 |
| 4-4655 | H | H | H | A2 | J1 | Q154 |
| 4-4656 | H | H | Cl | A2 | J1 | Q154 |
| 4-4657 | H | H | Br | A2 | J1 | Q154 |
| 4-4658 | H | H | CH₂F | A2 | J1 | Q154 |
| 4-4659 | H | H | CHF2 | A2 | J1 | Q154 |
| 4-4660 | H | H | Et | A2 | J1 | Q154 |
| 4-4661 | H | H | Pr | A2 | J1 | Q154 |
| 4-4662 | H | H | cPr | A2 | J1 | Q154 |
| 4-4663 | H | H | ethynyl | A2 | J1 | Q154 |
| 4-4664 | F | H | Me | A2 | J1 | Q154 |
| 4-4665 | F | F | Me | A2 | J1 | Q154 |
| 4-4666 | Me | H | Me | A2 | J1 | Q154 |
| 4-4667 | Me | Me | Me | A2 | J1 | Q154 |
| 4-4668 | H | H | H | A6 | J1 | Q154 |
| 4-4669 | H | H | Cl | A6 | J1 | Q154 |
| 4-4670 | H | H | Br | A6 | J1 | Q154 |
| 4-4671 | H | H | CH₂F | A6 | J1 | Q154 |
| 4-4672 | H | H | CHF2 | A6 | J1 | Q154 |
| 4-4673 | H | H | Et | A6 | J1 | Q154 |
| 4-4674 | H | H | Pr | A6 | J1 | Q154 |
| 4-4675 | H | H | cPr | A6 | J1 | Q154 |
| 4-4676 | H | H | ethynyl | A6 | J1 | Q154 |
| 4-4677 | F | H | Me | A6 | J1 | Q154 |
| 4-4678 | F | F | Me | A6 | J1 | Q154 |
| 4-4679 | Me | H | Me | A6 | J1 | Q154 |
| 4-4680 | Me | Me | Me | A6 | J1 | Q154 |
| 4-4681 | H | H | H | A9 | J1 | Q154 |
| 4-4682 | H | H | Cl | A9 | J1 | Q154 |
| 4-4683 | H | H | Br | A9 | J1 | Q154 |
| 4-4684 | H | H | CH₂F | A9 | J1 | Q154 |
| 4-4685 | H | H | CHF2 | A9 | J1 | Q154 |
| 4-4686 | H | H | Et | A9 | J1 | Q154 |
| 4-4687 | H | H | Pr | A9 | J1 | Q154 |
| 4-4688 | H | H | cPr | A9 | J1 | Q154 |
| 4-4689 | H | H | ethynyl | A9 | J1 | Q154 |
| 4-4690 | F | H | Me | A9 | J1 | Q154 |
| 4-4691 | F | F | Me | A9 | J1 | Q154 |
| 4-4692 | Me | H | Me | A9 | J1 | Q154 |
| 4-4693 | Me | Me | Me | A9 | J1 | Q154 |
| 4-4694 | H | H | H | A11 | J1 | Q154 |
| 4-4695 | H | H | Cl | A11 | J1 | Q154 |
| 4-4696 | H | H | Br | A11 | J1 | Q154 |
| 4-4697 | H | H | CH₂F | A11 | J1 | Q154 |
| 4-4698 | H | H | CHF2 | A11 | J1 | Q154 |
| 4-4699 | H | H | Et | A11 | J1 | Q154 |
| 4-4700 | H | H | Pr | A11 | J1 | Q154 |
| 4-4701 | H | H | cPr | A11 | J1 | Q154 |
| 4-4702 | H | H | ethynyl | A11 | J1 | Q154 |
| 4-4703 | F | H | Me | A11 | J1 | Q154 |
| 4-4704 | F | F | Me | A11 | J1 | Q154 |
| 4-4705 | Me | H | Me | A11 | J1 | Q154 |
| 4-4706 | Me | Me | Me | A11 | J1 | Q154 |
| 4-4707 | H | H | H | A12 | J1 | Q154 |
| 4-4708 | H | H | Cl | A12 | J1 | Q154 |
| 4-4709 | H | H | Br | A12 | J1 | Q154 |
| 4-4710 | H | H | CH₂F | A12 | J1 | Q154 |
| 4-4711 | H | H | CHF2 | A12 | J1 | Q154 |
| 4-4712 | H | H | Et | A12 | J1 | Q154 |
| 4-4713 | H | H | Pr | A12 | J1 | Q154 |
| 4-4714 | H | H | cPr | A12 | J1 | Q154 |
| 4-4715 | H | H | ethynyl | A12 | J1 | Q154 |
| 4-4716 | F | H | Me | A12 | J1 | Q154 |
| 4-4717 | F | F | Me | A12 | J1 | Q154 |
| 4-4718 | Me | H | Me | A12 | J1 | Q154 |
| 4-4719 | Me | Me | Me | A12 | J1 | Q154 |
| 4-4720 | H | H | H | A19 | J1 | Q154 |
| 4-4721 | H | H | Cl | A19 | J1 | Q154 |
| 4-4722 | H | H | Br | A19 | J1 | Q154 |
| 4-4723 | H | H | CH₂F | A19 | J1 | Q154 |
| 4-4724 | H | H | CHF2 | A19 | J1 | Q154 |
| 4-4725 | H | H | Et | A19 | J1 | Q154 |
| 4-4726 | H | H | Pr | A19 | J1 | Q154 |
| 4-4727 | H | H | cPr | A19 | J1 | Q154 |
| 4-4728 | H | H | ethynyl | A19 | J1 | Q154 |
| 4-4729 | F | H | Me | A19 | J1 | Q154 |
| 4-4730 | F | F | Me | A19 | J1 | Q154 |
| 4-4731 | Me | H | Me | A19 | J1 | Q154 |
| 4-4732 | Me | Me | Me | A19 | J1 | Q154 |
| 4-4733 | H | H | H | A20 | J1 | Q154 |
| 4-4734 | H | H | Cl | A20 | J1 | Q154 |
| 4-4735 | H | H | Br | A20 | J1 | Q154 |
| 4-4736 | H | H | CH₂F | A20 | J1 | Q154 |
| 4-4737 | H | H | CHF2 | A20 | J1 | Q154 |
| 4-4738 | H | H | Et | A20 | J1 | Q154 |
| 4-4739 | H | H | Pr | A20 | J1 | Q154 |
| 4-4740 | H | H | cPr | A20 | J1 | Q154 |
| 4-4741 | H | H | ethynyl | A20 | J1 | Q154 |
| 4-4742 | F | H | Me | A20 | J1 | Q154 |
| 4-4743 | F | F | Me | A20 | J1 | Q154 |
| 4-4744 | Me | H | Me | A20 | J1 | Q154 |
| 4-4745 | Me | Me | Me | A20 | J1 | Q154 |
| 4-4746 | H | H | H | A21 | J1 | Q154 |
| 4-4747 | H | H | Cl | A21 | J1 | Q154 |
| 4-4748 | H | H | Br | A21 | J1 | Q154 |
| 4-4749 | H | H | CH₂F | A21 | J1 | Q154 |
| 4-4750 | H | H | CHF2 | A21 | J1 | Q154 |
| 4-4751 | H | H | Et | A21 | J1 | Q154 |
| 4-4752 | H | H | Pr | A21 | J1 | Q154 |
| 4-4753 | H | H | cPr | A21 | J1 | Q154 |
| 4-4754 | H | H | ethynyl | A21 | J1 | Q154 |
| 4-4755 | F | H | Me | A21 | J1 | Q154 |
| 4-4756 | F | F | Me | A21 | J1 | Q154 |
| 4-4757 | Me | H | Me | A21 | J1 | Q154 |
| 4-4758 | Me | Me | Me | A21 | J1 | Q154 |
| 4-4759 | H | H | H | A22 | J1 | Q154 |
| 4-4760 | H | H | Cl | A22 | J1 | Q154 |
| 4-4761 | H | H | Br | A22 | J1 | Q154 |
| 4-4762 | H | H | CH₂F | A22 | J1 | Q154 |
| 4-4763 | H | H | CHF2 | A22 | J1 | Q154 |
| 4-4764 | H | H | Et | A22 | J1 | Q154 |
| 4-4765 | H | H | Pr | A22 | J1 | Q154 |
| 4-4766 | H | H | cPr | A22 | J1 | Q154 |
| 4-4767 | H | H | ethynyl | A22 | J1 | Q154 |
| 4-4768 | F | H | Me | A22 | J1 | Q154 |
| 4-4769 | F | F | Me | A22 | J1 | Q154 |
| 4-4770 | Me | H | Me | A22 | J1 | Q154 |
| 4-4771 | Me | Me | Me | A22 | J1 | Q154 |
| 4-4772 | H | H | H | A26 | J1 | Q154 |
| 4-4773 | H | H | Cl | A26 | J1 | Q154 |
| 4-4774 | H | H | Br | A26 | J1 | Q154 |
| 4-4775 | H | H | CH₂F | A26 | J1 | Q154 |
| 4-4776 | H | H | CHF2 | A26 | J1 | Q154 |
| 4-4777 | H | H | Et | A26 | J1 | Q154 |
| 4-4778 | H | H | Pr | A26 | J1 | Q154 |
| 4-4779 | H | H | cPr | A26 | J1 | Q154 |
| 4-4780 | H | H | ethynyl | A26 | J1 | Q154 |
| 4-4781 | F | H | Me | A26 | J1 | Q154 |
| 4-4782 | F | F | Me | A26 | J1 | Q154 |
| 4-4783 | Me | H | Me | A26 | J1 | Q154 |
| 4-4784 | Me | Me | Me | A26 | J1 | Q154 |
| 4-4785 | H | H | H | A27 | J1 | Q154 |
| 4-4786 | H | H | Cl | A27 | J1 | Q154 |
| 4-4787 | H | H | Br | A27 | J1 | Q154 |
| 4-4788 | H | H | CH₂F | A27 | J1 | Q154 |
| 4-4789 | H | H | CHF2 | A27 | J1 | Q154 |
| 4-4790 | H | H | Et | A27 | J1 | Q154 |
| 4-4791 | H | H | Pr | A27 | J1 | Q154 |
| 4-4792 | H | H | cPr | A27 | J1 | Q154 |
| 4-4793 | H | H | ethynyl | A27 | J1 | Q154 |
| 4-4794 | F | H | Me | A27 | J1 | Q154 |
| 4-4795 | F | F | Me | A27 | J1 | Q154 |
| 4-4796 | Me | H | Me | A27 | J1 | Q154 |
| 4-4797 | Me | Me | Me | A27 | J1 | Q154 |
| 4-4798 | H | H | H | A28 | J1 | Q154 |
| 4-4799 | H | H | Cl | A28 | J1 | Q154 |
| 4-4800 | H | H | Br | A28 | J1 | Q154 |
| 4-4801 | H | H | CH₂F | A28 | J1 | Q154 |
| 4-4802 | H | H | CHF2 | A28 | J1 | Q154 |
| 4-4803 | H | H | Et | A28 | J1 | Q154 |
| 4-4804 | H | H | Pr | A28 | J1 | Q154 |
| 4-4805 | H | H | cPr | A28 | J1 | Q154 |
| 4-4806 | H | H | ethynyl | A28 | J1 | Q154 |
| 4-4807 | F | H | Me | A28 | J1 | Q154 |
| 4-4808 | F | F | Me | A28 | J1 | Q154 |
| 4-4809 | Me | H | Me | A28 | J1 | Q154 |
| 4-4810 | Me | Me | Me | A28 | J1 | Q154 |
| 4-4811 | H | H | H | A29 | J1 | Q154 |
| 4-4812 | H | H | Cl | A29 | J1 | Q154 |
| 4-4813 | H | H | Br | A29 | J1 | Q154 |
| 4-4814 | H | H | CH₂F | A29 | J1 | Q154 |
| 4-4815 | H | H | CHF2 | A29 | J1 | Q154 |
| 4-4816 | H | H | Et | A29 | J1 | Q154 |
| 4-4817 | H | H | Pr | A29 | J1 | Q154 |
| 4-4818 | H | H | cPr | A29 | J1 | Q154 |
| 4-4819 | H | H | ethynyl | A29 | J1 | Q154 |
| 4-4820 | F | H | Me | A29 | J1 | Q154 |
| 4-4821 | F | F | Me | A29 | J1 | Q154 |
| 4-4822 | Me | H | Me | A29 | J1 | Q154 |
| 4-4823 | Me | Me | Me | A29 | J1 | Q154 |
| 4-4824 | H | H | H | A30 | J1 | Q154 |
| 4-4825 | H | H | Cl | A30 | J1 | Q154 |
| 4-4826 | H | H | Br | A30 | J1 | Q154 |
| 4-4827 | H | H | CH₂F | A30 | J1 | Q154 |
| 4-4828 | H | H | CHF2 | A30 | J1 | Q154 |
| 4-4829 | H | H | Et | A30 | J1 | Q154 |
| 4-4830 | H | H | Pr | A30 | J1 | Q154 |
| 4-4831 | H | H | cPr | A30 | J1 | Q154 |
| 4-4832 | H | H | ethynyl | A30 | J1 | Q154 |
| 4-4833 | F | H | Me | A30 | J1 | Q154 |
| 4-4834 | F | F | Me | A30 | J1 | Q154 |
| 4-4835 | Me | H | Me | A30 | J1 | Q154 |
| 4-4836 | Me | Me | Me | A30 | J1 | Q154 |
| 4-4837 | H | H | H | A31 | J1 | Q154 |
| 4-4838 | H | H | Cl | A31 | J1 | Q154 |
| 4-4839 | H | H | Br | A31 | J1 | Q154 |
| 4-4840 | H | H | CH₂F | A31 | J1 | Q154 |
| 4-4841 | H | H | CHF2 | A31 | J1 | Q154 |
| 4-4842 | H | H | Et | A31 | J1 | Q154 |
| 4-4843 | H | H | Pr | A31 | J1 | Q154 |
| 4-4844 | H | H | cPr | A31 | J1 | Q154 |
| 4-4845 | H | H | ethynyl | A31 | J1 | Q154 |
| 4-4846 | F | H | Me | A31 | J1 | Q154 |
| 4-4847 | F | F | Me | A31 | J1 | Q154 |
| 4-4848 | Me | H | Me | A31 | J1 | Q154 |
| 4-4849 | Me | Me | Me | A31 | J1 | Q154 |
| 4-4850 | H | H | H | A51 | J1 | Q154 |
| 4-4851 | H | H | Cl | A51 | J1 | Q154 |
| 4-4852 | H | H | Br | A51 | J1 | Q154 |
| 4-4853 | H | H | CH₂F | A51 | J1 | Q154 |
| 4-4854 | H | H | CHF2 | A51 | J1 | Q154 |
| 4-4855 | H | H | Et | A51 | J1 | Q154 |
| 4-4856 | H | H | Pr | A51 | J1 | Q154 |
| 4-4857 | H | H | cPr | A51 | J1 | Q154 |
| 4-4858 | H | H | ethynyl | A51 | J1 | Q154 |
| 4-4859 | F | H | Me | A51 | J1 | Q154 |
| 4-4860 | F | F | Me | A51 | J1 | Q154 |
| 4-4861 | Me | H | Me | A51 | J1 | Q154 |
| 4-4862 | Me | Me | Me | A51 | J1 | Q154 |
| 4-4863 | H | H | H | A1 | J2 | Q160 |
| 4-4864 | H | H | Cl | A1 | J2 | Q160 |
| 4-4865 | H | H | Br | A1 | J2 | Q160 |
| 4-4866 | H | H | CH₂F | A1 | J2 | Q160 |
| 4-4867 | H | H | CHF2 | A1 | J2 | Q160 |
| 4-4868 | H | H | Et | A1 | J2 | Q160 |
| 4-4869 | H | H | Pr | A1 | J2 | Q160 |
| 4-4870 | H | H | cPr | A1 | J2 | Q160 |
| 4-4871 | H | H | ethynyl | A1 | J2 | Q160 |
| 4-4872 | F | H | Me | A1 | J2 | Q160 |
| 4-4873 | F | F | Me | A1 | J2 | Q160 |
| 4-4874 | Me | H | Me | A1 | J2 | Q160 |
| 4-4875 | Me | Me | Me | A1 | J2 | Q160 |
| 4-4876 | H | H | H | A2 | J2 | Q160 |
| 4-4877 | H | H | Cl | A2 | J2 | Q160 |
| 4-4878 | H | H | Br | A2 | J2 | Q160 |
| 4-4879 | H | H | CH₂F | A2 | J2 | Q160 |
| 4-4880 | H | H | CHF2 | A2 | J2 | Q160 |
| 4-4881 | H | H | Et | A2 | J2 | Q160 |
| 4-4882 | H | H | Pr | A2 | J2 | Q160 |
| 4-4883 | H | H | cPr | A2 | J2 | Q160 |
| 4-4884 | H | H | ethynyl | A2 | J2 | Q160 |
| 4-4885 | F | H | Me | A2 | J2 | Q160 |
| 4-4886 | F | F | Me | A2 | J2 | Q160 |
| 4-4887 | Me | H | Me | A2 | J2 | Q160 |
| 4-4888 | Me | Me | Me | A2 | J2 | Q160 |
| 4-4889 | H | H | H | A6 | J2 | Q160 |
| 4-4890 | H | H | Cl | A6 | J2 | Q160 |
| 4-4891 | H | H | Br | A6 | J2 | Q160 |
| 4-4892 | H | H | CH₂F | A6 | J2 | Q160 |
| 4-4893 | H | H | CHF2 | A6 | J2 | Q160 |
| 4-4894 | H | H | Et | A6 | J2 | Q160 |
| 4-4895 | H | H | Pr | A6 | J2 | Q160 |
| 4-4896 | H | H | cPr | A6 | J2 | Q160 |
| 4-4897 | H | H | ethynyl | A6 | J2 | Q160 |
| 4-4898 | F | H | Me | A6 | J2 | Q160 |
| 4-4899 | F | F | Me | A6 | J2 | Q160 |
| 4-4900 | Me | H | Me | A6 | J2 | Q160 |
| 4-4901 | Me | Me | Me | A6 | J2 | Q160 |
| 4-4902 | H | H | H | A9 | J2 | Q160 |
| 4-4903 | H | H | Cl | A9 | J2 | Q160 |
| 4-4904 | H | H | Br | A9 | J2 | Q160 |
| 4-4905 | H | H | CH₂F | A9 | J2 | Q160 |
| 4-4906 | H | H | CHF2 | A9 | J2 | Q160 |
| 4-4907 | H | H | Et | A9 | J2 | Q160 |
| 4-4908 | H | H | Pr | A9 | J2 | Q160 |
| 4-4909 | H | H | cPr | A9 | J2 | Q160 |
| 4-4910 | H | H | ethynyl | A9 | J2 | Q160 |
| 4-4911 | F | H | Me | A9 | J2 | Q160 |
| 4-4912 | F | F | Me | A9 | J2 | Q160 |
| 4-4913 | Me | H | Me | A9 | J2 | Q160 |
| 4-4914 | Me | Me | Me | A9 | J2 | Q160 |
| 4-4915 | H | H | H | A11 | J2 | Q160 |
| 4-4916 | H | H | Cl | A11 | J2 | Q160 |
| 4-4917 | H | H | Br | A11 | J2 | Q160 |
| 4-4918 | H | H | CH₂F | A11 | J2 | Q160 |
| 4-4919 | H | H | CHF2 | A11 | J2 | Q160 |
| 4-4920 | H | H | Et | A11 | J2 | Q160 |
| 4-4921 | H | H | Pr | A11 | J2 | Q160 |
| 4-4922 | H | H | cPr | A11 | J2 | Q160 |
| 4-4923 | H | H | ethynyl | A11 | J2 | Q160 |
| 4-4924 | F | H | Me | A11 | J2 | Q160 |
| 4-4925 | F | F | Me | A11 | J2 | Q160 |
| 4-4926 | Me | H | Me | A11 | J2 | Q160 |
| 4-4927 | Me | Me | Me | A11 | J2 | Q160 |
| 4-4928 | H | H | H | A12 | J2 | Q160 |
| 4-4929 | H | H | Cl | A12 | J2 | Q160 |
| 4-4930 | H | H | Br | A12 | J2 | Q160 |
| 4-4931 | H | H | CH₂F | A12 | J2 | Q160 |
| 4-4932 | H | H | CHF2 | A12 | J2 | Q160 |
| 4-4933 | H | H | Et | A12 | J2 | Q160 |
| 4-4934 | H | H | Pr | A12 | J2 | Q160 |
| 4-4935 | H | H | cPr | A12 | J2 | Q160 |
| 4-4936 | H | H | ethynyl | A12 | J2 | Q160 |
| 4-4937 | F | H | Me | A12 | J2 | Q160 |
| 4-4938 | F | F | Me | A12 | J2 | Q160 |
| 4-4939 | Me | H | Me | A12 | J2 | Q160 |
| 4-4940 | Me | Me | Me | A12 | J2 | Q160 |
| 4-4941 | H | H | H | A19 | J2 | Q160 |
| 4-4942 | H | H | Cl | A19 | J2 | Q160 |
| 4-4943 | H | H | Br | A19 | J2 | Q160 |
| 4-4944 | H | H | CH₂F | A19 | J2 | Q160 |
| 4-4945 | H | H | CHF2 | A19 | J2 | Q160 |
| 4-4946 | H | H | Et | A19 | J2 | Q160 |
| 4-4947 | H | H | Pr | A19 | J2 | Q160 |
| 4-4948 | H | H | cPr | A19 | J2 | Q160 |
| 4-4949 | H | H | ethynyl | A19 | J2 | Q160 |
| 4-4950 | F | H | Me | A19 | J2 | Q160 |
| 4-4951 | F | F | Me | A19 | J2 | Q160 |
| 4-4952 | Me | H | Me | A19 | J2 | Q160 |
| 4-4953 | Me | Me | Me | A19 | J2 | Q160 |
| 4-4954 | H | H | H | A20 | J2 | Q160 |
| 4-4955 | H | H | Cl | A20 | J2 | Q160 |
| 4-4956 | H | H | Br | A20 | J2 | Q160 |
| 4-4957 | H | H | CH₂F | A20 | J2 | Q160 |
| 4-4958 | H | H | CHF2 | A20 | J2 | Q160 |
| 4-4959 | H | H | Et | A20 | J2 | Q160 |
| 4-4960 | H | H | Pr | A20 | J2 | Q160 |
| 4-4961 | H | H | cPr | A20 | J2 | Q160 |
| 4-4962 | H | H | ethynyl | A20 | J2 | Q160 |
| 4-4963 | F | H | Me | A20 | J2 | Q160 |
| 4-4964 | F | F | Me | A20 | J2 | Q160 |
| 4-4965 | Me | H | Me | A20 | J2 | Q160 |
| 4-4966 | Me | Me | Me | A20 | J2 | Q160 |
| 4-4967 | H | H | H | A21 | J2 | Q160 |
| 4-4968 | H | H | Cl | A21 | J2 | Q160 |
| 4-4969 | H | H | Br | A21 | J2 | Q160 |
| 4-4970 | H | H | CH₂F | A21 | J2 | Q160 |
| 4-4971 | H | H | CHF2 | A21 | J2 | Q160 |
| 4-4972 | H | H | Et | A21 | J2 | Q160 |
| 4-4973 | H | H | Pr | A21 | J2 | Q160 |
| 4-4974 | H | H | cPr | A21 | J2 | Q160 |
| 4-4975 | H | H | ethynyl | A21 | J2 | Q160 |
| 4-4976 | F | H | Me | A21 | J2 | Q160 |
| 4-4977 | F | F | Me | A21 | J2 | Q160 |
| 4-4978 | Me | H | Me | A21 | J2 | Q160 |
| 4-4979 | Me | Me | Me | A21 | J2 | Q160 |
| 4-4980 | H | H | H | A22 | J2 | Q160 |
| 4-4981 | H | H | Cl | A22 | J2 | Q160 |
| 4-4982 | H | H | Br | A22 | J2 | Q160 |
| 4-4983 | H | H | CH₂F | A22 | J2 | Q160 |
| 4-4984 | H | H | CHF2 | A22 | J2 | Q160 |
| 4-4985 | H | H | Et | A22 | J2 | Q160 |
| 4-4986 | H | H | Pr | A22 | J2 | Q160 |
| 4-4987 | H | H | cPr | A22 | J2 | Q160 |
| 4-4988 | H | H | ethynyl | A22 | J2 | Q160 |
| 4-4989 | F | H | Me | A22 | J2 | Q160 |
| 4-4990 | F | F | Me | A22 | J2 | Q160 |
| 4-4991 | Me | H | Me | A22 | J2 | Q160 |
| 4-4992 | Me | Me | Me | A22 | J2 | Q160 |
| 4-4993 | H | H | H | A26 | J2 | Q160 |
| 4-4994 | H | H | Cl | A26 | J2 | Q160 |
| 4-4995 | H | H | Br | A26 | J2 | Q160 |
| 4-4996 | H | H | CH₂F | A26 | J2 | Q160 |
| 4-4997 | H | H | CHF2 | A26 | J2 | Q160 |
| 4-4998 | H | H | Et | A26 | J2 | Q160 |
| 4-4999 | H | H | Pr | A26 | J2 | Q160 |
| 4-5000 | H | H | cPr | A26 | J2 | Q160 |
| 4-5001 | H | H | ethynyl | A26 | J2 | Q160 |
| 4-5002 | F | H | Me | A26 | J2 | Q160 |
| 4-5003 | F | F | Me | A26 | J2 | Q160 |
| 4-5004 | Me | H | Me | A26 | J2 | Q160 |
| 4-5005 | Me | Me | Me | A26 | J2 | Q160 |
| 4-5006 | H | H | H | A27 | J2 | Q160 |
| 4-5007 | H | H | Cl | A27 | J2 | Q160 |
| 4-5008 | H | H | Br | A27 | J2 | Q160 |
| 4-5009 | H | H | CH₂F | A27 | J2 | Q160 |
| 4-5010 | H | H | CHF2 | A27 | J2 | Q160 |
| 4-5011 | H | H | Et | A27 | J2 | Q160 |
| 4-5012 | H | H | Pr | A27 | J2 | Q160 |
| 4-5013 | H | H | cPr | A27 | J2 | Q160 |
| 4-5014 | H | H | ethynyl | A27 | J2 | Q160 |
| 4-5015 | F | H | Me | A27 | J2 | Q160 |
| 4-5016 | F | F | Me | A27 | J2 | Q160 |
| 4-5017 | Me | H | Me | A27 | J2 | Q160 |
| 4-5018 | Me | Me | Me | A27 | J2 | Q160 |
| 4-5019 | H | H | H | A28 | J2 | Q160 |
| 4-5020 | H | H | Cl | A28 | J2 | Q160 |
| 4-5021 | H | H | Br | A28 | J2 | Q160 |
| 4-5022 | H | H | CH₂F | A28 | J2 | Q160 |
| 4-5023 | H | H | CHF2 | A28 | J2 | Q160 |
| 4-5024 | H | H | Et | A28 | J2 | Q160 |
| 4-5025 | H | H | Pr | A28 | J2 | Q160 |
| 4-5026 | H | H | cPr | A28 | J2 | Q160 |
| 4-5027 | H | H | ethynyl | A28 | J2 | Q160 |
| 4-5028 | F | H | Me | A28 | J2 | Q160 |
| 4-5029 | F | F | Me | A28 | J2 | Q160 |
| 4-5030 | Me | H | Me | A28 | J2 | Q160 |
| 4-5031 | Me | Me | Me | A28 | J2 | Q160 |
| 4-5032 | H | H | H | A29 | J2 | Q160 |
| 4-5033 | H | H | Cl | A29 | J2 | Q160 |
| 4-5034 | H | H | Br | A29 | J2 | Q160 |
| 4-5035 | H | H | CH₂F | A29 | J2 | Q160 |
| 4-5036 | H | H | CHF2 | A29 | J2 | Q160 |
| 4-5037 | H | H | Et | A29 | J2 | Q160 |
| 4-5038 | H | H | Pr | A29 | J2 | Q160 |
| 4-5039 | H | H | cPr | A29 | J2 | Q160 |
| 4-5040 | H | H | ethynyl | A29 | J2 | Q160 |
| 4-5041 | F | H | Me | A29 | J2 | Q160 |
| 4-5042 | F | F | Me | A29 | J2 | Q160 |
| 4-5043 | Me | H | Me | A29 | J2 | Q160 |
| 4-5044 | Me | Me | Me | A29 | J2 | Q160 |
| 4-5045 | H | H | H | A30 | J2 | Q160 |
| 4-5046 | H | H | Cl | A30 | J2 | Q160 |
| 4-5047 | H | H | Br | A30 | J2 | Q160 |
| 4-5048 | H | H | CH₂F | A30 | J2 | Q160 |
| 4-5049 | H | H | CHF2 | A30 | J2 | Q160 |
| 4-5050 | H | H | Et | A30 | J2 | Q160 |
| 4-5051 | H | H | Pr | A30 | J2 | Q160 |
| 4-5052 | H | H | cPr | A30 | J2 | Q160 |
| 4-5053 | H | H | ethynyl | A30 | J2 | Q160 |
| 4-5054 | F | H | Me | A30 | J2 | Q160 |
| 4-5055 | F | F | Me | A30 | J2 | Q160 |
| 4-5056 | Me | H | Me | A30 | J2 | Q160 |
| 4-5057 | Me | Me | Me | A30 | J2 | Q160 |
| 4-5058 | H | H | H | A31 | J2 | Q160 |
| 4-5059 | H | H | Cl | A31 | J2 | Q160 |
| 4-5060 | H | H | Br | A31 | J2 | Q160 |
| 4-5061 | H | H | CH₂F | A31 | J2 | Q160 |
| 4-5062 | H | H | CHF2 | A31 | J2 | Q160 |
| 4-5063 | H | H | Et | A31 | J2 | Q160 |
| 4-5064 | H | H | Pr | A31 | J2 | Q160 |
| 4-5065 | H | H | cPr | A31 | J2 | Q160 |
| 4-5066 | H | H | ethynyl | A31 | J2 | Q160 |
| 4-5067 | F | H | Me | A31 | J2 | Q160 |
| 4-5068 | F | F | Me | A31 | J2 | Q160 |
| 4-5069 | Me | H | Me | A31 | J2 | Q160 |
| 4-5070 | Me | Me | Me | A31 | J2 | Q160 |
| 4-5071 | H | H | H | A51 | J2 | Q160 |
| 4-5072 | H | H | Cl | A51 | J2 | Q160 |
| 4-5073 | H | H | Br | A51 | J2 | Q160 |
| 4-5074 | H | H | CH₂F | A51 | J2 | Q160 |
| 4-5075 | H | H | CHF2 | A51 | J2 | Q160 |
| 4-5076 | H | H | Et | A51 | J2 | Q160 |
| 4-5077 | H | H | Pr | A51 | J2 | Q160 |
| 4-5078 | H | H | cPr | A51 | J2 | Q160 |
| 4-5079 | H | H | ethynyl | A51 | J2 | Q160 |
| 4-5080 | F | H | Me | A51 | J2 | Q160 |
| 4-5081 | F | F | Me | A51 | J2 | Q160 |
| 4-5082 | Me | H | Me | A51 | J2 | Q160 |
| 4-5083 | Me | Me | Me | A51 | J2 | Q160 |
| 4-5084 | H | H | H | A1 | J1 | Q160 |
| 4-5085 | H | H | Cl | A1 | J1 | Q160 |
| 4-5086 | H | H | Br | A1 | J1 | Q160 |
| 4-5087 | H | H | CH₂F | A1 | J1 | Q160 |
| 4-5088 | H | H | CHF2 | A1 | J1 | Q160 |
| 4-5089 | H | H | Et | A1 | J1 | Q160 |
| 4-5090 | H | H | Pr | A1 | J1 | Q160 |
| 4-5091 | H | H | cPr | A1 | J1 | Q160 |
| 4-5092 | H | H | ethynyl | A1 | J1 | Q160 |
| 4-5093 | F | H | Me | A1 | J1 | Q160 |
| 4-5094 | F | F | Me | A1 | J1 | Q160 |
| 4-5095 | Me | H | Me | A1 | J1 | Q160 |
| 4-5096 | Me | Me | Me | A1 | J1 | Q160 |
| 4-5097 | H | H | H | A2 | J1 | Q160 |
| 4-5098 | H | H | Cl | A2 | J1 | Q160 |
| 4-5099 | H | H | Br | A2 | J1 | Q160 |
| 4-5100 | H | H | CH₂F | A2 | J1 | Q160 |
| 4-5101 | H | H | CHF2 | A2 | J1 | Q160 |
| 4-5102 | H | H | Et | A2 | J1 | Q160 |
| 4-5103 | H | H | Pr | A2 | J1 | Q160 |
| 4-5104 | H | H | cPr | A2 | J1 | Q160 |
| 4-5105 | H | H | ethynyl | A2 | J1 | Q160 |
| 4-5106 | F | H | Me | A2 | J1 | Q160 |
| 4-5107 | F | F | Me | A2 | J1 | Q160 |
| 4-5108 | Me | H | Me | A2 | J1 | Q160 |
| 4-5109 | Me | Me | Me | A2 | J1 | Q160 |
| 4-5110 | H | H | H | A6 | J1 | Q160 |
| 4-5111 | H | H | Cl | A6 | J1 | Q160 |
| 4-5112 | H | H | Br | A6 | J1 | Q160 |
| 4-5113 | H | H | CH₂F | A6 | J1 | Q160 |
| 4-5114 | H | H | CHF2 | A6 | J1 | Q160 |
| 4-5115 | H | H | Et | A6 | J1 | Q160 |
| 4-5116 | H | H | Pr | A6 | J1 | Q160 |
| 4-5117 | H | H | cPr | A6 | J1 | Q160 |
| 4-5118 | H | H | ethynyl | A6 | J1 | Q160 |
| 4-5119 | F | H | Me | A6 | J1 | Q160 |
| 4-5120 | F | F | Me | A6 | J1 | Q160 |
| 4-5121 | Me | H | Me | A6 | J1 | Q160 |
| 4-5122 | Me | Me | Me | A6 | J1 | Q160 |
| 4-5123 | H | H | H | A9 | J1 | Q160 |
| 4-5124 | H | H | Cl | A9 | J1 | Q160 |
| 4-5125 | H | H | Br | A9 | J1 | Q160 |
| 4-5126 | H | H | CH₂F | A9 | J1 | Q160 |
| 4-5127 | H | H | CHF2 | A9 | J1 | Q160 |
| 4-5128 | H | H | Et | A9 | J1 | Q160 |
| 4-5129 | H | H | Pr | A9 | J1 | Q160 |
| 4-5130 | H | H | cPr | A9 | J1 | Q160 |
| 4-5131 | H | H | ethynyl | A9 | J1 | Q160 |
| 4-5132 | F | H | Me | A9 | J1 | Q160 |
| 4-5133 | F | F | Me | A9 | J1 | Q160 |
| 4-5134 | Me | H | Me | A9 | J1 | Q160 |
| 4-5135 | Me | Me | Me | A9 | J1 | Q160 |
| 4-5136 | H | H | H | A11 | J1 | Q160 |
| 4-5137 | H | H | Cl | A11 | J1 | Q160 |
| 4-5138 | H | H | Br | A11 | J1 | Q160 |
| 4-5139 | H | H | CH₂F | A11 | J1 | Q160 |
| 4-5140 | H | H | CHF2 | A11 | J1 | Q160 |
| 4-5141 | H | H | Et | A11 | J1 | Q160 |
| 4-5142 | H | H | Pr | A11 | J1 | Q160 |
| 4-5143 | H | H | cPr | A11 | J1 | Q160 |
| 4-5144 | H | H | ethynyl | A11 | J1 | Q160 |
| 4-5145 | F | H | Me | A11 | J1 | Q160 |
| 4-5146 | F | F | Me | A11 | J1 | Q160 |
| 4-5147 | Me | H | Me | A11 | J1 | Q160 |
| 4-5148 | Me | Me | Me | A11 | J1 | Q160 |
| 4-5149 | H | H | H | A12 | J1 | Q160 |
| 4-5150 | H | H | Cl | A12 | J1 | Q160 |
| 4-5151 | H | H | Br | A12 | J1 | Q160 |
| 4-5152 | H | H | CH₂F | A12 | J1 | Q160 |
| 4-5153 | H | H | CHF2 | A12 | J1 | Q160 |
| 4-5154 | H | H | Et | A12 | J1 | Q160 |
| 4-5155 | H | H | Pr | A12 | J1 | Q160 |
| 4-5156 | H | H | cPr | A12 | J1 | Q160 |
| 4-5157 | H | H | ethynyl | A12 | J1 | Q160 |
| 4-5158 | F | H | Me | A12 | J1 | Q160 |
| 4-5159 | F | F | Me | A12 | J1 | Q160 |
| 4-5160 | Me | H | Me | A12 | J1 | Q160 |
| 4-5161 | Me | Me | Me | A12 | J1 | Q160 |
| 4-5162 | H | H | H | A19 | J1 | Q160 |
| 4-5163 | H | H | Cl | A19 | J1 | Q160 |
| 4-5164 | H | H | Br | A19 | J1 | Q160 |
| 4-5165 | H | H | CH₂F | A19 | J1 | Q160 |
| 4-5166 | H | H | CHF2 | A19 | J1 | Q160 |
| 4-5167 | H | H | Et | A19 | J1 | Q160 |
| 4-5168 | H | H | Pr | A19 | J1 | Q160 |
| 4-5169 | H | H | cPr | A19 | J1 | Q160 |
| 4-5170 | H | H | ethynyl | A19 | J1 | Q160 |
| 4-5171 | F | H | Me | A19 | J1 | Q160 |
| 4-5172 | F | F | Me | A19 | J1 | Q160 |
| 4-5173 | Me | H | Me | A19 | J1 | Q160 |
| 4-5174 | Me | Me | Me | A19 | J1 | Q160 |
| 4-5175 | H | H | H | A20 | J1 | Q160 |
| 4-5176 | H | H | Cl | A20 | J1 | Q160 |
| 4-5177 | H | H | Br | A20 | J1 | Q160 |
| 4-5178 | H | H | CH₂F | A20 | J1 | Q160 |
| 4-5179 | H | H | CHF2 | A20 | J1 | Q160 |
| 4-5180 | H | H | Et | A20 | J1 | Q160 |
| 4-5181 | H | H | Pr | A20 | J1 | Q160 |
| 4-5182 | H | H | cPr | A20 | J1 | Q160 |
| 4-5183 | H | H | ethynyl | A20 | J1 | Q160 |
| 4-5184 | F | H | Me | A20 | J1 | Q160 |
| 4-5185 | F | F | Me | A20 | J1 | Q160 |
| 4-5186 | Me | H | Me | A20 | J1 | Q160 |
| 4-5187 | Me | Me | Me | A20 | J1 | Q160 |
| 4-5188 | H | H | H | A21 | J1 | Q160 |
| 4-5189 | H | H | Cl | A21 | J1 | Q160 |
| 4-5190 | H | H | Br | A21 | J1 | Q160 |
| 4-5191 | H | H | CH₂F | A21 | J1 | Q160 |
| 4-5192 | H | H | CHF2 | A21 | J1 | Q160 |
| 4-5193 | H | H | Et | A21 | J1 | Q160 |
| 4-5194 | H | H | Pr | A21 | J1 | Q160 |
| 4-5195 | H | H | cPr | A21 | J1 | Q160 |
| 4-5196 | H | H | ethynyl | A21 | J1 | Q160 |
| 4-5197 | F | H | Me | A21 | J1 | Q160 |
| 4-5198 | F | F | Me | A21 | J1 | Q160 |
| 4-5199 | Me | H | Me | A21 | J1 | Q160 |
| 4-5200 | Me | Me | Me | A21 | J1 | Q160 |
| 4-5201 | H | H | H | A22 | J1 | Q160 |
| 4-5202 | H | H | Cl | A22 | J1 | Q160 |
| 4-5203 | H | H | Br | A22 | J1 | Q160 |
| 4-5204 | H | H | CH₂F | A22 | J1 | Q160 |
| 4-5205 | H | H | CHF2 | A22 | J1 | Q160 |
| 4-5206 | H | H | Et | A22 | J1 | Q160 |
| 4-5207 | H | H | Pr | A22 | J1 | Q160 |
| 4-5208 | H | H | cPr | A22 | J1 | Q160 |
| 4-5209 | H | H | ethynyl | A22 | J1 | Q160 |
| 4-5210 | F | H | Me | A22 | J1 | Q160 |
| 4-5211 | F | F | Me | A22 | J1 | Q160 |
| 4-5212 | Me | H | Me | A22 | J1 | Q160 |
| 4-5213 | Me | Me | Me | A22 | J1 | Q160 |
| 4-5214 | H | H | H | A26 | J1 | Q160 |
| 4-5215 | H | H | Cl | A26 | J1 | Q160 |
| 4-5216 | H | H | Br | A26 | J1 | Q160 |
| 4-5217 | H | H | CH₂F | A26 | J1 | Q160 |
| 4-5218 | H | H | CHF2 | A26 | J1 | Q160 |
| 4-5219 | H | H | Et | A26 | J1 | Q160 |
| 4-5220 | H | H | Pr | A26 | J1 | Q160 |
| 4-5221 | H | H | cPr | A26 | J1 | Q160 |
| 4-5222 | H | H | ethynyl | A26 | J1 | Q160 |
| 4-5223 | F | H | Me | A26 | J1 | Q160 |
| 4-5224 | F | F | Me | A26 | J1 | Q160 |
| 4-5225 | Me | H | Me | A26 | J1 | Q160 |
| 4-5226 | Me | Me | Me | A26 | J1 | Q160 |
| 4-5227 | H | H | H | A27 | J1 | Q160 |
| 4-5228 | H | H | Cl | A27 | J1 | Q160 |
| 4-5229 | H | H | Br | A27 | J1 | Q160 |
| 4-5230 | H | H | CH₂F | A27 | J1 | Q160 |
| 4-5231 | H | H | CHF2 | A27 | J1 | Q160 |
| 4-5232 | H | H | Et | A27 | J1 | Q160 |
| 4-5233 | H | H | Pr | A27 | J1 | Q160 |
| 4-5234 | H | H | cPr | A27 | J1 | Q160 |
| 4-5235 | H | H | ethynyl | A27 | J1 | Q160 |
| 4-5236 | F | H | Me | A27 | J1 | Q160 |
| 4-5237 | F | F | Me | A27 | J1 | Q160 |
| 4-5238 | Me | H | Me | A27 | J1 | Q160 |
| 4-5239 | Me | Me | Me | A27 | J1 | Q160 |
| 4-5240 | H | H | H | A28 | J1 | Q160 |
| 4-5241 | H | H | Cl | A28 | J1 | Q160 |
| 4-5242 | H | H | Br | A28 | J1 | Q160 |
| 4-5243 | H | H | CH₂F | A28 | J1 | Q160 |
| 4-5244 | H | H | CHF2 | A28 | J1 | Q160 |
| 4-5245 | H | H | Et | A28 | J1 | Q160 |
| 4-5246 | H | H | Pr | A28 | J1 | Q160 |
| 4-5247 | H | H | cPr | A28 | J1 | Q160 |
| 4-5248 | H | H | ethynyl | A28 | J1 | Q160 |
| 4-5249 | F | H | Me | A28 | J1 | Q160 |
| 4-5250 | F | F | Me | A28 | J1 | Q160 |
| 4-5251 | Me | H | Me | A28 | J1 | Q160 |
| 4-5252 | Me | Me | Me | A28 | J1 | Q160 |
| 4-5253 | H | H | H | A29 | J1 | Q160 |
| 4-5254 | H | H | Cl | A29 | J1 | Q160 |
| 4-5255 | H | H | Br | A29 | J1 | Q160 |
| 4-5256 | H | H | CH₂F | A29 | J1 | Q160 |
| 4-5257 | H | H | CHF2 | A29 | J1 | Q160 |
| 4-5258 | H | H | Et | A29 | J1 | Q160 |
| 4-5259 | H | H | Pr | A29 | J1 | Q160 |
| 4-5260 | H | H | cPr | A29 | J1 | Q160 |
| 4-5261 | H | H | ethynyl | A29 | J1 | Q160 |
| 4-5262 | F | H | Me | A29 | J1 | Q160 |
| 4-5263 | F | F | Me | A29 | J1 | Q160 |
| 4-5264 | Me | H | Me | A29 | J1 | Q160 |
| 4-5265 | Me | Me | Me | A29 | J1 | Q160 |
| 4-5266 | H | H | H | A30 | J1 | Q160 |
| 4-5267 | H | H | Cl | A30 | J1 | Q160 |
| 4-5268 | H | H | Br | A30 | J1 | Q160 |
| 4-5269 | H | H | CH₂F | A30 | J1 | Q160 |
| 4-5270 | H | H | CHF2 | A30 | J1 | Q160 |
| 4-5271 | H | H | Et | A30 | J1 | Q160 |
| 4-5272 | H | H | Pr | A30 | J1 | Q160 |
| 4-5273 | H | H | cPr | A30 | J1 | Q160 |
| 4-5274 | H | H | ethynyl | A30 | J1 | Q160 |
| 4-5275 | F | H | Me | A30 | J1 | Q160 |
| 4-5276 | F | F | Me | A30 | J1 | Q160 |
| 4-5277 | Me | H | Me | A30 | J1 | Q160 |
| 4-5278 | Me | Me | Me | A30 | J1 | Q160 |
| 4-5279 | H | H | H | A31 | J1 | Q160 |
| 4-5280 | H | H | Cl | A31 | J1 | Q160 |
| 4-5281 | H | H | Br | A31 | J1 | Q160 |
| 4-5282 | H | H | CH₂F | A31 | J1 | Q160 |
| 4-5283 | H | H | CHF2 | A31 | J1 | Q160 |
| 4-5284 | H | H | Et | A31 | J1 | Q160 |
| 4-5285 | H | H | Pr | A31 | J1 | Q160 |
| 4-5286 | H | H | cPr | A31 | J1 | Q160 |
| 4-5287 | H | H | ethynyl | A31 | J1 | Q160 |
| 4-5288 | F | H | Me | A31 | J1 | Q160 |
| 4-5289 | F | F | Me | A31 | J1 | Q160 |
| 4-5290 | Me | H | Me | A31 | J1 | Q160 |
| 4-5291 | Me | Me | Me | A31 | J1 | Q160 |
| 4-5292 | H | H | H | A51 | J1 | Q160 |
| 4-5293 | H | H | Cl | A51 | J1 | Q160 |
| 4-5294 | H | H | Br | A51 | J1 | Q160 |
| 4-5295 | H | H | CH₂F | A51 | J1 | Q160 |
| 4-5296 | H | H | CHF2 | A51 | J1 | Q160 |
| 4-5297 | H | H | Et | A51 | J1 | Q160 |
| 4-5298 | H | H | Pr | A51 | J1 | Q160 |
| 4-5299 | H | H | cPr | A51 | J1 | Q160 |
| 4-5300 | H | H | ethynyl | A51 | J1 | Q160 |
| 4-5301 | F | H | Me | A51 | J1 | Q160 |
| 4-5302 | F | F | Me | A51 | J1 | Q160 |
| 4-5303 | Me | H | Me | A51 | J1 | Q160 |
| 4-5304 | Me | Me | Me | A51 | J1 | Q160 |
| 4-5305 | H | H | H | A¹ | J2 | Q221 |
| 4-5306 | H | H | Cl | A1 | J2 | Q221 |
| 4-5307 | H | H | Br | A1 | J2 | Q221 |
| 4-5308 | H | H | CH₂F | A1 | J2 | Q221 |
| 4-5309 | H | H | CHF2 | A1 | J2 | Q221 |
| 4-5310 | H | H | Et | A1 | J2 | Q221 |
| 4-5311 | H | H | Pr | A1 | J2 | Q221 |
| 4-5312 | H | H | cPr | A1 | J2 | Q221 |
| 4-5313 | H | H | ethynyl | A1 | J2 | Q221 |
| 4-5314 | F | H | Me | A1 | J2 | Q221 |
| 4-5315 | F | F | Me | A1 | J2 | Q221 |
| 4-5316 | Me | H | Me | A1 | J2 | Q221 |
| 4-5317 | Me | Me | Me | A1 | J2 | Q221 |
| 4-5318 | H | H | H | A2 | J2 | Q221 |
| 4-5319 | H | H | Cl | A2 | J2 | Q221 |
| 4-5320 | H | H | Br | A2 | J2 | Q221 |
| 4-5321 | H | H | CH₂F | A2 | J2 | Q221 |
| 4-5322 | H | H | CHF2 | A2 | J2 | Q221 |
| 4-5323 | H | H | Et | A2 | J2 | Q221 |
| 4-5324 | H | H | Pr | A2 | J2 | Q221 |
| 4-5325 | H | H | cPr | A2 | J2 | Q221 |
| 4-5326 | H | H | ethynyl | A2 | J2 | Q221 |
| 4-5327 | F | H | Me | A2 | J2 | Q221 |
| 4-5328 | F | F | Me | A2 | J2 | Q221 |
| 4-5329 | Me | H | Me | A2 | J2 | Q221 |
| 4-5330 | Me | Me | Me | A2 | J2 | Q221 |
| 4-5331 | H | H | H | A6 | J2 | Q221 |
| 4-5332 | H | H | Cl | A6 | J2 | Q221 |
| 4-5333 | H | H | Br | A6 | J2 | Q221 |
| 4-5334 | H | H | CH₂F | A6 | J2 | Q221 |
| 4-5335 | H | H | CHF2 | A6 | J2 | Q221 |
| 4-5336 | H | H | Et | A6 | J2 | Q221 |
| 4-5337 | H | H | Pr | A6 | J2 | Q221 |
| 4-5338 | H | H | cPr | A6 | J2 | Q221 |
| 4-5339 | H | H | ethynyl | A6 | J2 | Q221 |
| 4-5340 | F | H | Me | A6 | J2 | Q221 |
| 4-5341 | F | F | Me | A6 | J2 | Q221 |
| 4-5342 | Me | H | Me | A6 | J2 | Q221 |
| 4-5343 | Me | Me | Me | A6 | J2 | Q221 |
| 4-5344 | H | H | H | A9 | J2 | Q221 |
| 4-5345 | H | H | Cl | A9 | J2 | Q221 |
| 4-5346 | H | H | Br | A9 | J2 | Q221 |
| 4-5347 | H | H | CH₂F | A9 | J2 | Q221 |
| 4-5348 | H | H | CHF2 | A9 | J2 | Q221 |
| 4-5349 | H | H | Et | A9 | J2 | Q221 |
| 4-5350 | H | H | Pr | A9 | J2 | Q221 |
| 4-5351 | H | H | cPr | A9 | J2 | Q221 |
| 4-5352 | H | H | ethynyl | A9 | J2 | Q221 |
| 4-5353 | F | H | Me | A9 | J2 | Q221 |
| 4-5354 | F | F | Me | A9 | J2 | Q221 |
| 4-5355 | Me | H | Me | A9 | J2 | Q221 |
| 4-5356 | Me | Me | Me | A9 | J2 | Q221 |
| 4-5357 | H | H | H | A11 | J2 | Q221 |
| 4-5358 | H | H | Cl | A11 | J2 | Q221 |
| 4-5359 | H | H | Br | A11 | J2 | Q221 |
| 4-5360 | H | H | CH₂F | A11 | J2 | Q221 |
| 4-5361 | H | H | CHF2 | A11 | J2 | Q221 |
| 4-5362 | H | H | Et | A11 | J2 | Q221 |
| 4-5363 | H | H | Pr | A11 | J2 | Q221 |
| 4-5364 | H | H | cPr | A11 | J2 | Q221 |
| 4-5365 | H | H | ethynyl | A11 | J2 | Q221 |
| 4-5366 | F | H | Me | A11 | J2 | Q221 |
| 4-5367 | F | F | Me | A11 | J2 | Q221 |
| 4-5368 | Me | H | Me | A11 | J2 | Q221 |
| 4-5369 | Me | Me | Me | A11 | J2 | Q221 |
| 4-5370 | H | H | H | A12 | J2 | Q221 |
| 4-5371 | H | H | Cl | A12 | J2 | Q221 |
| 4-5372 | H | H | Br | A12 | J2 | Q221 |
| 4-5373 | H | H | CH₂F | A12 | J2 | Q221 |
| 4-5374 | H | H | CHF2 | A12 | J2 | Q221 |
| 4-5375 | H | H | Et | A12 | J2 | Q221 |
| 4-5376 | H | H | Pr | A12 | J2 | Q221 |
| 4-5377 | H | H | cPr | A12 | J2 | Q221 |
| 4-5378 | H | H | ethynyl | A12 | J2 | Q221 |
| 4-5379 | F | H | Me | A12 | J2 | Q221 |
| 4-5380 | F | F | Me | A12 | J2 | Q221 |
| 4-5381 | Me | H | Me | A12 | J2 | Q221 |
| 4-5382 | Me | Me | Me | A12 | J2 | Q221 |
| 4-5383 | H | H | H | A19 | J2 | Q221 |
| 4-5384 | H | H | Cl | A19 | J2 | Q221 |
| 4-5385 | H | H | Br | A19 | J2 | Q221 |
| 4-5386 | H | H | CH₂F | A19 | J2 | Q221 |
| 4-5387 | H | H | CHF2 | A19 | J2 | Q221 |
| 4-5388 | H | H | Et | A19 | J2 | Q221 |
| 4-5389 | H | H | Pr | A19 | J2 | Q221 |
| 4-5390 | H | H | cPr | A19 | J2 | Q221 |
| 4-5391 | H | H | ethynyl | A19 | J2 | Q221 |
| 4-5392 | F | H | Me | A19 | J2 | Q221 |
| 4-5393 | F | F | Me | A19 | J2 | Q221 |
| 4-5394 | Me | H | Me | A19 | J2 | Q221 |
| 4-5395 | Me | Me | Me | A19 | J2 | Q221 |
| 4-5396 | H | H | H | A20 | J2 | Q221 |
| 4-5397 | H | H | Cl | A20 | J2 | Q221 |
| 4-5398 | H | H | Br | A20 | J2 | Q221 |
| 4-5399 | H | H | CH₂F | A20 | J2 | Q221 |
| 4-5400 | H | H | CHF2 | A20 | J2 | Q221 |
| 4-5401 | H | H | Et | A20 | J2 | Q221 |
| 4-5402 | H | H | Pr | A20 | J2 | Q221 |
| 4-5403 | H | H | cPr | A20 | J2 | Q221 |
| 4-5404 | H | H | ethynyl | A20 | J2 | Q221 |
| 4-5405 | F | H | Me | A20 | J2 | Q221 |
| 4-5406 | F | F | Me | A20 | J2 | Q221 |
| 4-5407 | Me | H | Me | A20 | J2 | Q221 |
| 4-5408 | Me | Me | Me | A20 | J2 | Q221 |
| 4-5409 | H | H | H | A21 | J2 | Q221 |
| 4-5410 | H | H | Cl | A21 | J2 | Q221 |
| 4-5411 | H | H | Br | A21 | J2 | Q221 |
| 4-5412 | H | H | CH₂F | A21 | J2 | Q221 |
| 4-5413 | H | H | CHF2 | A21 | J2 | Q221 |
| 4-5414 | H | H | Et | A21 | J2 | Q221 |
| 4-5415 | H | H | Pr | A21 | J2 | Q221 |
| 4-5416 | H | H | cPr | A21 | J2 | Q221 |
| 4-5417 | H | H | ethynyl | A21 | J2 | Q221 |
| 4-5418 | F | H | Me | A21 | J2 | Q221 |
| 4-5419 | F | F | Me | A21 | J2 | Q221 |
| 4-5420 | Me | H | Me | A21 | J2 | Q221 |
| 4-5421 | Me | Me | Me | A21 | J2 | Q221 |
| 4-5422 | H | H | H | A22 | J2 | Q221 |
| 4-5423 | H | H | Cl | A22 | J2 | Q221 |
| 4-5424 | H | H | Br | A22 | J2 | Q221 |
| 4-5425 | H | H | CH₂F | A22 | J2 | Q221 |
| 4-5426 | H | H | CHF2 | A22 | J2 | Q221 |
| 4-5427 | H | H | Et | A22 | J2 | Q221 |
| 4-5428 | H | H | Pr | A22 | J2 | Q221 |
| 4-5429 | H | H | cPr | A22 | J2 | Q221 |
| 4-5430 | H | H | ethynyl | A22 | J2 | Q221 |
| 4-5431 | F | H | Me | A22 | J2 | Q221 |
| 4-5432 | F | F | Me | A22 | J2 | Q221 |
| 4-5433 | Me | H | Me | A22 | J2 | Q221 |
| 4-5434 | Me | Me | Me | A22 | J2 | Q221 |
| 4-5435 | H | H | H | A26 | J2 | Q221 |
| 4-5436 | H | H | Cl | A26 | J2 | Q221 |
| 4-5437 | H | H | Br | A26 | J2 | Q221 |
| 4-5438 | H | H | CH₂F | A26 | J2 | Q221 |
| 4-5439 | H | H | CHF2 | A26 | J2 | Q221 |
| 4-5440 | H | H | Et | A26 | J2 | Q221 |
| 4-5441 | H | H | Pr | A26 | J2 | Q221 |
| 4-5442 | H | H | cPr | A26 | J2 | Q221 |
| 4-5443 | H | H | ethynyl | A26 | J2 | Q221 |
| 4-5444 | F | H | Me | A26 | J2 | Q221 |
| 4-5445 | F | F | Me | A26 | J2 | Q221 |
| 4-5446 | Me | H | Me | A26 | J2 | Q221 |
| 4-5447 | Me | Me | Me | A26 | J2 | Q221 |
| 4-5448 | H | H | H | A27 | J2 | Q221 |
| 4-5449 | H | H | Cl | A27 | J2 | Q221 |
| 4-5450 | H | H | Br | A27 | J2 | Q221 |
| 4-5451 | H | H | CH₂F | A27 | J2 | Q221 |
| 4-5452 | H | H | CHF2 | A27 | J2 | Q221 |
| 4-5453 | H | H | Et | A27 | J2 | Q221 |
| 4-5454 | H | H | Pr | A27 | J2 | Q221 |
| 4-5455 | H | H | cPr | A27 | J2 | Q221 |
| 4-5456 | H | H | ethynyl | A27 | J2 | Q221 |
| 4-5457 | F | H | Me | A27 | J2 | Q221 |
| 4-5458 | F | F | Me | A27 | J2 | Q221 |
| 4-5459 | Me | H | Me | A27 | J2 | Q221 |
| 4-5460 | Me | Me | Me | A27 | J2 | Q221 |
| 4-5461 | H | H | H | A28 | J2 | Q221 |
| 4-5462 | H | H | Cl | A28 | J2 | Q221 |
| 4-5463 | H | H | Br | A28 | J2 | Q221 |
| 4-5464 | H | H | CH₂F | A28 | J2 | Q221 |
| 4-5465 | H | H | CHF2 | A28 | J2 | Q221 |
| 4-5466 | H | H | Et | A28 | J2 | Q221 |
| 4-5467 | H | H | Pr | A28 | J2 | Q221 |
| 4-5468 | H | H | cPr | A28 | J2 | Q221 |
| 4-5469 | H | H | ethynyl | A28 | J2 | Q221 |
| 4-5470 | F | H | Me | A28 | J2 | Q221 |
| 4-5471 | F | F | Me | A28 | J2 | Q221 |
| 4-5472 | Me | H | Me | A28 | J2 | Q221 |
| 4-5473 | Me | Me | Me | A28 | J2 | Q221 |
| 4-5474 | H | H | H | A29 | J2 | Q221 |
| 4-5475 | H | H | Cl | A29 | J2 | Q221 |
| 4-5476 | H | H | Br | A29 | J2 | Q221 |
| 4-5477 | H | H | CH₂F | A29 | J2 | Q221 |
| 4-5478 | H | H | CHF2 | A29 | J2 | Q221 |
| 4-5479 | H | H | Et | A29 | J2 | Q221 |
| 4-5480 | H | H | Pr | A29 | J2 | Q221 |
| 4-5481 | H | H | cPr | A29 | J2 | Q221 |
| 4-5482 | H | H | ethynyl | A29 | J2 | Q221 |
| 4-5483 | F | H | Me | A29 | J2 | Q221 |
| 4-5484 | F | F | Me | A29 | J2 | Q221 |
| 4-5485 | Me | H | Me | A29 | J2 | Q221 |
| 4-5486 | Me | Me | Me | A29 | J2 | Q221 |
| 4-5487 | H | H | H | A30 | J2 | Q221 |
| 4-5488 | H | H | Cl | A30 | J2 | Q221 |
| 4-5489 | H | H | Br | A30 | J2 | Q221 |
| 4-5490 | H | H | CH₂F | A30 | J2 | Q221 |
| 4-5491 | H | H | CHF2 | A30 | J2 | Q221 |
| 4-5492 | H | H | Et | A30 | J2 | Q221 |
| 4-5493 | H | H | Pr | A30 | J2 | Q221 |
| 4-5494 | H | H | cPr | A30 | J2 | Q221 |
| 4-5495 | H | H | ethynyl | A30 | J2 | Q221 |
| 4-5496 | F | H | Me | A30 | J2 | Q221 |
| 4-5497 | F | F | Me | A30 | J2 | Q221 |
| 4-5498 | Me | H | Me | A30 | J2 | Q221 |
| 4-5499 | Me | Me | Me | A30 | J2 | Q221 |
| 4-5500 | H | H | H | A31 | J2 | Q221 |
| 4-5501 | H | H | Cl | A31 | J2 | Q221 |
| 4-5502 | H | H | Br | A31 | J2 | Q221 |
| 4-5503 | H | H | CH₂F | A31 | J2 | Q221 |
| 4-5504 | H | H | CHF2 | A31 | J2 | Q221 |
| 4-5505 | H | H | Et | A31 | J2 | Q221 |
| 4-5506 | H | H | Pr | A31 | J2 | Q221 |
| 4-5507 | H | H | cPr | A31 | J2 | Q221 |
| 4-5508 | H | H | ethynyl | A31 | J2 | Q221 |
| 4-5509 | F | H | Me | A31 | J2 | Q221 |
| 4-5510 | F | F | Me | A31 | J2 | Q221 |
| 4-5511 | Me | H | Me | A31 | J2 | Q221 |
| 4-5512 | Me | Me | Me | A31 | J2 | Q221 |
| 4-5513 | H | H | H | A51 | J2 | Q221 |
| 4-5514 | H | H | Cl | A51 | J2 | Q221 |
| 4-5515 | H | H | Br | A51 | J2 | Q221 |
| 4-5516 | H | H | CH₂F | A51 | J2 | Q221 |
| 4-5517 | H | H | CHF2 | A51 | J2 | Q221 |
| 4-5518 | H | H | Et | A51 | J2 | Q221 |
| 4-5519 | H | H | Pr | A51 | J2 | Q221 |
| 4-5520 | H | H | cPr | A51 | J2 | Q221 |
| 4-5521 | H | H | ethynyl | A51 | J2 | Q221 |
| 4-5522 | F | H | Me | A51 | J2 | Q221 |
| 4-5523 | F | F | Me | A51 | J2 | Q221 |
| 4-5524 | Me | H | Me | A51 | J2 | Q221 |
| 4-5525 | Me | Me | Me | A51 | J2 | Q221 |
| 4-5526 | H | H | H | A1 | J1 | Q221 |
| 4-5527 | H | H | Cl | A1 | J1 | Q221 |
| 4-5528 | H | H | Br | A1 | J1 | Q221 |
| 4-5529 | H | H | CH₂F | A1 | J1 | Q221 |
| 4-5530 | H | H | CHF2 | A1 | J1 | Q221 |
| 4-5531 | H | H | Et | A1 | J1 | Q221 |
| 4-5532 | H | H | Pr | A1 | J1 | Q221 |
| 4-5533 | H | H | cPr | A1 | J1 | Q221 |
| 4-5534 | H | H | ethynyl | A1 | J1 | Q221 |
| 4-5535 | F | H | Me | A1 | J1 | Q221 |
| 4-5536 | F | F | Me | A1 | J1 | Q221 |
| 4-5537 | Me | H | Me | A1 | J1 | Q221 |
| 4-5538 | Me | Me | Me | A1 | J1 | Q221 |
| 4-5539 | H | H | H | A2 | J1 | Q221 |
| 4-5540 | H | H | Cl | A2 | J1 | Q221 |
| 4-5541 | H | H | Br | A2 | J1 | Q221 |
| 4-5542 | H | H | CH₂F | A2 | J1 | Q221 |
| 4-5543 | H | H | CHF2 | A2 | J1 | Q221 |
| 4-5544 | H | H | Et | A2 | J1 | Q221 |
| 4-5545 | H | H | Pr | A2 | J1 | Q221 |
| 4-5546 | H | H | cPr | A2 | J1 | Q221 |
| 4-5547 | H | H | ethynyl | A2 | J1 | Q221 |
| 4-5548 | F | H | Me | A2 | J1 | Q221 |
| 4-5549 | F | F | Me | A2 | J1 | Q221 |
| 4-5550 | Me | H | Me | A2 | J1 | Q221 |
| 4-5551 | Me | Me | Me | A2 | J1 | Q221 |
| 4-5552 | H | H | H | A6 | J1 | Q221 |
| 4-5553 | H | H | Cl | A6 | J1 | Q221 |
| 4-5554 | H | H | Br | A6 | J1 | Q221 |
| 4-5555 | H | H | CH₂F | A6 | J1 | Q221 |
| 4-5556 | H | H | CHF2 | A6 | J1 | Q221 |
| 4-5557 | H | H | Et | A6 | J1 | Q221 |
| 4-5558 | H | H | Pr | A6 | J1 | Q221 |
| 4-5559 | H | H | cPr | A6 | J1 | Q221 |
| 4-5560 | H | H | ethynyl | A6 | J1 | Q221 |
| 4-5561 | F | H | Me | A6 | J1 | Q221 |
| 4-5562 | F | F | Me | A6 | J1 | Q221 |
| 4-5563 | Me | H | Me | A6 | J1 | Q221 |
| 4-5564 | Me | Me | Me | A6 | J1 | Q221 |
| 4-5565 | H | H | H | A9 | J1 | Q221 |
| 4-5566 | H | H | Cl | A9 | J1 | Q221 |
| 4-5567 | H | H | Br | A9 | J1 | Q221 |
| 4-5568 | H | H | CH₂F | A9 | J1 | Q221 |
| 4-5569 | H | H | CHF2 | A9 | J1 | Q221 |
| 4-5570 | H | H | Et | A9 | J1 | Q221 |
| 4-5571 | H | H | Pr | A9 | J1 | Q221 |
| 4-5572 | H | H | cPr | A9 | J1 | Q221 |
| 4-5573 | H | H | ethynyl | A9 | J1 | Q221 |
| 4-5574 | F | H | Me | A9 | J1 | Q221 |
| 4-5575 | F | F | Me | A9 | J1 | Q221 |
| 4-5576 | Me | H | Me | A9 | J1 | Q221 |
| 4-5577 | Me | Me | Me | A9 | J1 | Q221 |
| 4-5578 | H | H | H | A11 | J1 | Q221 |
| 4-5579 | H | H | Cl | A11 | J1 | Q221 |
| 4-5580 | H | H | Br | A11 | J1 | Q221 |
| 4-5581 | H | H | CH₂F | A11 | J1 | Q221 |
| 4-5582 | H | H | CHF2 | A11 | J1 | Q221 |
| 4-5583 | H | H | Et | A11l | J1 | Q221 |
| 4-5584 | H | H | cPr | A11 | J1 | Q221 |
| 4-5585 | H | H | cPr | A11 | J1 | Q221 |
| 4-5586 | H | H | ethynyl | A11 | J1 | Q221 |
| 4-5587 | F | H | Me | A11 | J1 | Q221 |
| 4-5588 | F | F | Me | A11 | J1 | Q221 |
| 4-5589 | Me | H | Me | A11 | J1 | Q221 |
| 4-5590 | Me | Me | Me | A11 | J1 | Q221 |
| 4-5591 | H | H | H | A12 | J1 | Q221 |
| 4-5592 | H | H | Cl | A12 | J1 | Q221 |
| 4-5593 | H | H | Br | A12 | J1 | Q221 |
| 4-5594 | H | H | CH₂F | A12 | J1 | Q221 |
| 4-5595 | H | H | CHF2 | A12 | J1 | Q221 |
| 4-5596 | H | H | Et | A12 | J1 | Q221 |
| 4-5597 | H | H | Pr | A12 | J1 | Q221 |
| 4-5598 | H | H | cPr | A12 | J1 | Q221 |
| 4-5599 | H | H | ethynyl | A12 | J1 | Q221 |
| 4-5600 | F | H | Me | A12 | J1 | Q221 |
| 4-5601 | F | F | Me | A12 | J1 | Q221 |
| 4-5602 | Me | H | Me | A12 | J1 | Q221 |
| 4-5603 | Me | Me | Me | A12 | J1 | Q221 |
| 4-5604 | H | H | H | A19 | J1 | Q221 |
| 4-5605 | H | H | Cl | A19 | J1 | Q221 |
| 4-5606 | H | H | Br | A19 | J1 | Q221 |
| 4-5607 | H | H | CH₂F | A19 | J1 | Q221 |
| 4-5608 | H | H | CHF2 | A19 | J1 | Q221 |
| 4-5609 | H | H | Et | A19 | J1 | Q221 |
| 4-5610 | H | H | Pr | A19 | J1 | Q221 |
| 4-5611 | H | H | cPr | A19 | J1 | Q221 |
| 4-5612 | H | H | ethynyl | A19 | J1 | Q221 |
| 4-5613 | F | H | Me | A19 | J1 | Q221 |
| 4-5614 | F | F | Me | A19 | J1 | Q221 |
| 4-5615 | Me | H | Me | A19 | J1 | Q221 |
| 4-5616 | Me | Me | Me | A19 | J1 | Q221 |
| 4-5617 | H | H | H | A20 | J1 | Q221 |
| 4-5618 | H | H | Cl | A20 | J1 | Q221 |
| 4-5619 | H | H | Br | A20 | J1 | Q221 |
| 4-5620 | H | H | CH₂F | A20 | J1 | Q221 |
| 4-5621 | H | H | CHF2 | A20 | J1 | Q221 |
| 4-5622 | H | H | Et | A20 | J1 | Q221 |
| 4-5623 | H | H | Pr | A20 | J1 | Q221 |
| 4-5624 | H | H | cPr | A20 | J1 | Q221 |
| 4-5625 | H | H | ethynyl | A20 | J1 | Q221 |
| 4-5626 | F | H | Me | A20 | J1 | Q221 |
| 4-5627 | F | F | Me | A20 | J1 | Q221 |
| 4-5628 | Me | H | Me | A20 | J1 | Q221 |
| 4-5629 | Me | Me | Me | A20 | J1 | Q221 |
| 4-5630 | H | H | H | A21 | J1 | Q221 |
| 4-5631 | H | H | Cl | A21 | J1 | Q221 |
| 4-5632 | H | H | Br | A21 | J1 | Q221 |
| 4-5633 | H | H | CH₂F | A21 | J1 | Q221 |
| 4-5634 | H | H | CHF2 | A21 | J1 | Q221 |
| 4-5635 | H | H | Et | A21 | J1 | Q221 |
| 4-5636 | H | H | Pr | A21 | J1 | Q221 |
| 4-5637 | H | H | cPr | A21 | J1 | Q221 |
| 4-5638 | H | H | ethynyl | A21 | J1 | Q221 |
| 4-5639 | F | H | Me | A21 | J1 | Q221 |
| 4-5640 | F | F | Me | A21 | J1 | Q221 |
| 4-5641 | Me | H | Me | A21 | J1 | Q221 |
| 4-5642 | Me | Me | Me | A21 | J1 | Q221 |
| 4-5643 | H | H | H | A22 | J1 | Q221 |
| 4-5644 | H | H | Cl | A22 | J1 | Q221 |
| 4-5645 | H | H | Br | A22 | J1 | Q221 |
| 4-5646 | H | H | CH₂F | A22 | J1 | Q221 |
| 4-5647 | H | H | CHF2 | A22 | J1 | Q221 |
| 4-5648 | H | H | Et | A22 | J1 | Q221 |
| 4-5649 | H | H | Pr | A22 | J1 | Q221 |
| 4-5650 | H | H | cPr | A22 | J1 | Q221 |
| 4-5651 | H | H | ethynyl | A22 | J1 | Q221 |
| 4-5652 | F | H | Me | A22 | J1 | Q221 |
| 4-5653 | F | F | Me | A22 | J1 | Q221 |
| 4-5654 | Me | H | Me | A22 | J1 | Q221 |
| 4-5655 | Me | Me | Me | A22 | J1 | Q221 |
| 4-5656 | H | H | H | A26 | J1 | Q221 |
| 4-5657 | H | H | Cl | A26 | J1 | Q221 |
| 4-5658 | H | H | Br | A26 | J1 | Q221 |
| 4-5659 | H | H | CH₂F | A26 | J1 | Q221 |
| 4-5660 | H | H | CHF2 | A26 | J1 | Q221 |
| 4-5661 | H | H | Et | A26 | J1 | Q221 |
| 4-5662 | H | H | Pr | A26 | J1 | Q221 |
| 4-5663 | H | H | cPr | A26 | J1 | Q221 |
| 4-5664 | H | H | ethynyl | A26 | J1 | Q221 |
| 4-5665 | F | H | Me | A26 | J1 | Q221 |
| 4-5666 | F | F | Me | A26 | J1 | Q221 |
| 4-5667 | Me | H | Me | A26 | J1 | Q221 |
| 4-5668 | Me | Me | Me | A26 | J1 | Q221 |
| 4-5669 | H | H | H | A27 | J1 | Q221 |
| 4-5670 | H | H | Cl | A27 | J1 | Q221 |
| 4-5671 | H | H | Br | A27 | J1 | Q221 |
| 4-5672 | H | H | CH₂F | A27 | J1 | Q221 |
| 4-5673 | H | H | CHF2 | A27 | J1 | Q221 |
| 4-5674 | H | H | Et | A27 | J1 | Q221 |
| 4-5675 | H | H | Pr | A27 | J1 | Q221 |
| 4-5676 | H | H | cPr | A27 | J1 | Q221 |
| 4-5677 | H | H | ethynyl | A27 | J1 | Q221 |
| 4-5678 | F | H | Me | A27 | J1 | Q221 |
| 4-5679 | F | F | Me | A27 | J1 | Q221 |
| 4-5680 | Me | H | Me | A27 | J1 | Q221 |
| 4-5681 | Me | Me | Me | A27 | J1 | Q221 |
| 4-5682 | H | H | H | A28 | J1 | Q221 |
| 4-5683 | H | H | Cl | A28 | J1 | Q221 |
| 4-5684 | H | H | Br | A28 | J1 | Q221 |
| 4-5685 | H | H | CH₂F | A28 | J1 | Q221 |
| 4-5686 | H | H | CHF2 | A28 | J1 | Q221 |
| 4-5687 | H | H | Et | A28 | J1 | Q221 |
| 4-5688 | H | H | Pr | A28 | J1 | Q221 |
| 4-5689 | H | H | cPr | A28 | J1 | Q221 |
| 4-5690 | H | H | ethynyl | A28 | J1 | Q221 |
| 4-5691 | F | H | Me | A28 | J1 | Q221 |
| 4-5692 | F | F | Me | A28 | J1 | Q221 |
| 4-5693 | Me | H | Me | A28 | J1 | Q221 |
| 4-5694 | Me | Me | Me | A28 | J1 | Q221 |
| 4-5695 | H | H | H | A29 | J1 | Q221 |
| 4-5696 | H | H | Cl | A29 | J1 | Q221 |
| 4-5697 | H | H | Br | A29 | J1 | Q221 |
| 4-5698 | H | H | CH₂F | A29 | J1 | Q221 |
| 4-5699 | H | H | CHF2 | A29 | J1 | Q221 |
| 4-5700 | H | H | Et | A29 | J1 | Q221 |
| 4-5701 | H | H | Pr | A29 | J1 | Q221 |
| 4-5702 | H | H | cPr | A29 | J1 | Q221 |
| 4-5703 | H | H | ethynyl | A29 | J1 | Q221 |
| 4-5704 | F | H | Me | A29 | J1 | Q221 |
| 4-5705 | F | F | Me | A29 | J1 | Q221 |
| 4-5706 | Me | H | Me | A29 | J1 | Q221 |
| 4-5707 | Me | Me | Me | A29 | J1 | Q221 |
| 4-5708 | H | H | H | A30 | J1 | Q221 |
| 4-5709 | H | H | Cl | A30 | J1 | Q221 |
| 4-5710 | H | H | Br | A30 | J1 | Q221 |
| 4-5711 | H | H | CH₂F | A30 | J1 | Q221 |
| 4-5712 | H | H | CHF2 | A30 | J1 | Q221 |
| 4-5713 | H | H | Et | A30 | J1 | Q221 |
| 4-5714 | H | H | Pr | A30 | J1 | Q221 |
| 4-5715 | H | H | cPr | A30 | J1 | Q221 |
| 4-5716 | H | H | ethynyl | A30 | J1 | Q221 |
| 4-5717 | F | H | Me | A30 | J1 | Q221 |
| 4-5718 | F | F | Me | A30 | J1 | Q221 |
| 4-5719 | Me | H | Me | A30 | J1 | Q221 |
| 4-5720 | Me | Me | Me | A30 | J1 | Q221 |
| 4-5721 | H | H | H | A31 | J1 | Q221 |
| 4-5722 | H | H | Cl | A31 | J1 | Q221 |
| 4-5723 | H | H | Br | A31 | J1 | Q221 |
| 4-5724 | H | H | CH₂F | A31 | J1 | Q221 |
| 4-5725 | H | H | CHF2 | A31 | J1 | Q221 |
| 4-5726 | H | H | Et | A31 | J1 | Q221 |
| 4-5727 | H | H | Pr | A31 | J1 | Q221 |
| 4-5728 | H | H | cPr | A31 | J1 | Q221 |
| 4-5729 | H | H | ethynyl | A31 | J1 | Q221 |
| 4-5730 | F | H | Me | A31 | J1 | Q221 |
| 4-5731 | F | F | Me | A31 | J1 | Q221 |
| 4-5732 | Me | H | Me | A31 | J1 | Q221 |
| 4-5733 | Me | Me | Me | A31 | J1 | Q221 |
| 4-5734 | H | H | H | A51 | J1 | Q221 |
| 4-5735 | H | H | Cl | A51 | J1 | Q221 |
| 4-5736 | H | H | Br | A51 | J1 | Q221 |
| 4-5737 | H | H | CH₂F | A51 | J1 | Q221 |
| 4-5738 | H | H | CHF2 | A51 | J1 | Q221 |
| 4-5739 | H | H | Et | A51 | J1 | Q221 |
| 4-5740 | H | H | Pr | A51 | J1 | Q221 |
| 4-5741 | H | H | cPr | A51 | J1 | Q221 |
| 4-5742 | H | H | ethynyl | A51 | J1 | Q221 |
| 4-5743 | F | H | Me | A51 | J1 | Q221 |
| 4-5744 | F | F | Me | A51 | J1 | Q221 |
| 4-5745 | Me | H | Me | A51 | J1 | Q221 |
| 4-5746 | Me | Me | Me | A51 | J1 | Q221 |

In addition, NMR data for compounds of the formula (I) according to the invention were generated. “NMRs” of the exemplary compounds were in each case measured as ¹H-NMR spectrum at 300 or 400 MHz (CDCl₃) (¹H nuclear magnetic resonance data). Characteristic chemical shifts δ (ppm) for some exemplary compounds are shown below:

NMR Compound 4-3678 (CDCl₃, 400 MHz, δ in ppm):

1.41 (d, 3H); 2.33 (s, 3H); 2.66 (s, 3H); 3.62 (s, 3H); 3.71 (q, 2H); 7.33 (dd, 1H); 7.53 (dd, 1H); 7.77 (td, 1H); 8.66 (dt, 1H).

NMR Compound 4-3899 (CDCl₃, 400 MHz, δ in ppm):

1.48 (d, 3H); 2.43 (s, 3H); 2.71 (s, 3H); 3.71 (q, 2H); 7.52 (td, 1H); 7.96 (m, 2H); 8.68 (dt, 1H).

TABLE 5
Compounds of the formula (I-2)
(I-2)

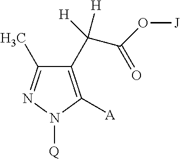

In the table below, for the compounds of the formula (I-2), the
examples of the radicals A, J and Q in the respective column are
referred to by the abbreviations (example numbers) as defined in
Tables 1 to 3 for the radicals A, J and Q, respectively.

| No. | A | J | Q |
|---|---|---|---|
| 5-1 | A1 | J1 | Q23 |
| 5-2 | A2 | J1 | Q23 |
| 5-3 | A6 | J1 | Q23 |
| 5-4 | A9 | J1 | Q23 |
| 5-5 | A11 | J1 | Q23 |
| 5-6 | A12 | J1 | Q23 |
| 5-7 | A16 | J1 | Q23 |
| 5-8 | A19 | J1 | Q23 |
| 5-9 | A20 | J1 | Q23 |
| 5-10 | A21 | J1 | Q23 |
| 5-11 | A22 | J1 | Q23 |
| 5-12 | A25 | J1 | Q23 |
| 5-13 | A26 | J1 | Q23 |
| 5-14 | A27 | J1 | Q23 |
| 5-15 | A28 | J1 | Q23 |
| 5-16 | A29 | J1 | Q23 |
| 5-17 | A30 | J1 | Q23 |
| 5-18 | A31 | J1 | Q23 |
| 5-19 | A51 | J1 | Q23 |
| 5-20 | A57 | J1 | Q23 |
| 5-21 | A58 | J1 | Q23 |
| 5-22 | A1 | J1 | Q52 |
| 5-23 | A2 | J1 | Q52 |
| 5-24 | A6 | J1 | Q52 |
| 5-25 | A9 | J1 | Q52 |
| 5-26 | A11 | J1 | Q52 |
| 5-27 | A12 | J1 | Q52 |
| 5-28 | A16 | J1 | Q52 |
| 5-29 | A19 | J1 | Q52 |
| 5-30 | A20 | J1 | Q52 |
| 5-31 | A21 | J1 | Q52 |
| 5-32 | A22 | J1 | Q52 |
| 5-33 | A25 | J1 | Q52 |
| 5-34 | A26 | J1 | Q52 |
| 5-35 | A27 | J1 | Q52 |
| 5-36 | A28 | J1 | Q52 |
| 5-37 | A29 | J1 | Q52 |
| 5-38 | A30 | J1 | Q52 |
| 5-39 | A31 | J1 | Q52 |
| 5-40 | A51 | J1 | Q52 |
| 5-41 | A57 | J1 | Q52 |
| 5-42 | A58 | J1 | Q52 |
| 5-43 | A1 | J1 | Q56 |
| 5-44 | A2 | J1 | Q56 |
| 5-45 | A6 | J1 | Q56 |
| 5-46 | A9 | J1 | Q56 |
| 5-47 | A11 | J1 | Q56 |
| 5-48 | A12 | J1 | Q56 |
| 5-49 | A16 | J1 | Q56 |
| 5-50 | A19 | J1 | Q56 |
| 5-51 | A20 | J1 | Q56 |
| 5-52 | A21 | J1 | Q56 |
| 5-53 | A22 | J1 | Q56 |
| 5-54 | A25 | J1 | Q56 |
| 5-55 | A26 | J1 | Q56 |
| 5-56 | A27 | J1 | Q56 |
| 5-57 | A28 | J1 | Q56 |
| 5-58 | A29 | J1 | Q56 |
| 5-59 | A30 | J1 | Q56 |
| 5-60 | A31 | J1 | Q56 |
| 5-61 | A51 | J1 | Q56 |
| 5-62 | A57 | J1 | Q56 |
| 5-63 | A58 | J1 | Q56 |
| 5-64 | A1 | J1 | Q58 |
| 5-65 | A2 | J1 | Q58 |
| 5-66 | A6 | J1 | Q58 |
| 5-67 | A9 | J1 | Q58 |
| 5-68 | A11 | J1 | Q58 |
| 5-69 | A12 | J1 | Q58 |
| 5-70 | A16 | J1 | Q58 |
| 5-71 | A19 | J1 | Q58 |
| 5-72 | A20 | J1 | Q58 |
| 5-73 | A21 | J1 | Q58 |
| 5-74 | A22 | J1 | Q58 |
| 5-75 | A25 | J1 | Q58 |
| 5-76 | A26 | J1 | Q58 |
| 5-77 | A27 | J1 | Q58 |
| 5-78 | A28 | J1 | Q58 |
| 5-79 | A29 | J1 | Q58 |
| 5-80 | A30 | J1 | Q58 |
| 5-81 | A31 | J1 | Q58 |
| 5-82 | A51 | J1 | Q58 |
| 5-83 | A57 | J1 | Q58 |
| 5-84 | A58 | J1 | Q58 |
| 5-85 | A1 | J1 | Q59 |
| 5-86 | A2 | J1 | Q59 |
| 5-87 | A6 | J1 | Q59 |
| 5-88 | A9 | J1 | Q59 |
| 5-89 | A11 | J1 | Q59 |
| 5-90 | A12 | J1 | Q59 |
| 5-91 | A16 | J1 | Q59 |
| 5-92 | A19 | J1 | Q59 |
| 5-93 | A20 | J1 | Q59 |
| 5-94 | A21 | J1 | Q59 |
| 5-95 | A22 | J1 | Q59 |
| 5-96 | A25 | J1 | Q59 |
| 5-97 | A26 | J1 | Q59 |
| 5-98 | A27 | J1 | Q59 |
| 5-99 | A28 | J1 | Q59 |
| 5-100 | A29 | J1 | Q59 |
| 5-101 | A30 | J1 | Q59 |
| 5-102 | A31 | J1 | Q59 |
| 5-103 | A51 | J1 | Q59 |
| 5-104 | A57 | J1 | Q59 |
| 5-105 | A58 | J1 | Q59 |
| 5-106 | A1 | J1 | Q65 |
| 5-107 | A2 | J1 | Q65 |
| 5-108 | A6 | J1 | Q65 |
| 5-109 | A9 | J1 | Q65 |
| 5-110 | A11 | J1 | Q65 |
| 5-111 | A12 | J1 | Q65 |
| 5-112 | A16 | J1 | Q65 |
| 5-113 | A19 | J1 | Q65 |
| 5-114 | A20 | J1 | Q65 |
| 5-115 | A21 | J1 | Q65 |
| 5-116 | A22 | J1 | Q65 |
| 5-117 | A25 | J1 | Q65 |
| 5-118 | A26 | J1 | Q65 |
| 5-119 | A27 | J1 | Q65 |
| 5-120 | A28 | J1 | Q65 |
| 5-121 | A29 | J1 | Q65 |
| 5-122 | A30 | J1 | Q65 |
| 5-123 | A31 | J1 | Q65 |
| 5-124 | A51 | J1 | Q65 |
| 5-125 | A57 | J1 | Q65 |
| 5-126 | A58 | J1 | Q65 |
| 5-127 | A1 | J1 | Q66 |
| 5-128 | A2 | J1 | Q66 |
| 5-129 | A6 | J1 | Q66 |
| 5-130 | A9 | J1 | Q66 |
| 5-131 | A11 | J1 | Q66 |
| 5-132 | A12 | J1 | Q66 |
| 5-133 | A16 | J1 | Q66 |
| 5-134 | A19 | J1 | Q66 |
| 5-135 | A20 | J1 | Q66 |
| 5-136 | A21 | J1 | Q66 |
| 5-137 | A22 | J1 | Q66 |
| 5-138 | A25 | J1 | Q66 |
| 5-139 | A26 | J1 | Q66 |
| 3-140 | A27 | J1 | Q66 |
| 5-141 | A28 | J1 | Q66 |
| 5-142 | A29 | J1 | Q66 |
| 5-143 | A30 | J1 | Q66 |
| 5-144 | A31 | J1 | Q66 |
| 5-145 | A51 | J1 | Q66 |
| 5-146 | A57 | J1 | Q66 |
| 5-147 | A58 | J1 | Q66 |
| 5-148 | A1 | J1 | Q70 |
| 5-149 | A2 | J1 | Q70 |
| 5-150 | A6 | J1 | Q70 |
| 5-151 | A9 | J1 | Q70 |
| 5-152 | A11 | J1 | Q70 |
| 5-153 | A12 | J1 | Q70 |
| 5-154 | A16 | J1 | Q70 |
| 5-155 | A19 | J1 | Q70 |
| 5-156 | A20 | J1 | Q70 |
| 5-157 | A21 | J1 | Q70 |
| 5-158 | A22 | J1 | Q70 |
| 5-159 | A25 | J1 | Q70 |
| 5-160 | A26 | J1 | Q70 |
| 5-161 | A27 | J1 | Q70 |
| 5-162 | A28 | J1 | Q70 |
| 5-163 | A29 | J1 | Q70 |
| 5-164 | A30 | J1 | Q70 |
| 5-165 | A31 | J1 | Q70 |
| 5-166 | A51 | J1 | Q70 |
| 5-167 | A57 | J1 | Q70 |
| 5-168 | A58 | J1 | Q70 |
| 5-169 | A1 | J1 | Q78 |
| 5-170 | A2 | J1 | Q78 |
| 5-171 | A6 | J1 | Q78 |
| 5-172 | A9 | J1 | Q78 |
| 5-173 | A11 | J1 | Q78 |
| 5-174 | A12 | J1 | Q78 |
| 5-175 | A16 | J1 | Q78 |
| 5-176 | A19 | J1 | Q78 |
| 5-177 | A20 | J1 | Q78 |
| 5-178 | A21 | J1 | Q78 |
| 5-179 | A22 | J1 | Q78 |
| 5-180 | A25 | J1 | Q78 |
| 5-181 | A26 | J1 | Q78 |
| 5-182 | A27 | J1 | Q78 |
| 5-183 | A28 | J1 | Q78 |
| 5-184 | A29 | J1 | Q78 |
| 5-185 | A30 | J1 | Q78 |
| 5-186 | A31 | J1 | Q78 |
| 5-187 | A51 | J1 | Q78 |
| 5-188 | A57 | J1 | Q78 |
| 5-189 | A58 | J1 | Q78 |
| 5-190 | A1 | J1 | Q79 |
| 5-191 | A2 | J1 | Q79 |
| 5-192 | A6 | J1 | Q79 |
| 5-193 | A9 | J1 | Q79 |
| 5-194 | A11 | J1 | Q79 |
| 5-195 | A12 | J1 | Q79 |
| 5-196 | A16 | J1 | Q79 |
| 5-197 | A19 | J1 | Q79 |
| 5-198 | A20 | J1 | Q79 |
| 5-199 | A21 | J1 | Q79 |
| 5-200 | A22 | J1 | Q79 |
| 5-201 | A25 | J1 | Q79 |
| 5-202 | A26 | J1 | Q79 |
| 5-203 | A27 | J1 | Q79 |
| 5-204 | A28 | J1 | Q79 |
| 5-205 | A29 | J1 | Q79 |
| 5-206 | A30 | J1 | Q79 |
| 5-207 | A31 | J1 | Q79 |
| 5-208 | A51 | J1 | Q79 |
| 5-209 | A57 | J1 | Q79 |
| 5-210 | A58 | J1 | Q79 |
| 5-211 | A1 | J1 | Q80 |
| 5-212 | A2 | J1 | Q80 |
| 5-213 | A6 | J1 | Q80 |
| 5-214 | A9 | J1 | Q80 |
| 5-215 | A11 | J1 | Q80 |
| 5-216 | A12 | J1 | Q80 |
| 5-217 | A16 | J1 | Q80 |
| 5-218 | A19 | J1 | Q80 |
| 5-219 | A20 | J1 | Q80 |
| 3-220 | A21 | J1 | Q80 |
| 5-221 | A22 | J1 | Q80 |
| 5-222 | A25 | J1 | Q80 |
| 5-223 | A26 | J1 | Q80 |
| 5-224 | A27 | J1 | Q80 |
| 5-225 | A28 | J1 | Q80 |
| 5-226 | A29 | J1 | Q80 |
| 5-227 | A30 | J1 | Q80 |
| 5-228 | A31 | J1 | Q80 |
| 5-229 | A51 | J1 | Q80 |
| 5-230 | A57 | J1 | Q80 |
| 5-231 | A58 | J1 | Q80 |
| 5-232 | A1 | J1 | Q81 |
| 5-233 | A2 | J1 | Q81 |
| 5-234 | A6 | J1 | Q81 |
| 5-235 | A9 | J1 | Q81 |
| 5-236 | A11 | J1 | Q81 |
| 5-237 | A12 | J1 | Q81 |
| 5-238 | A16 | J1 | Q81 |
| 5-239 | A19 | J1 | Q81 |
| 5-240 | A20 | J1 | Q81 |
| 5-241 | A21 | J1 | Q81 |
| 5-242 | A22 | J1 | Q81 |
| 5-243 | A25 | J1 | Q81 |
| 5-244 | A26 | J1 | Q81 |
| 5-245 | A27 | J1 | Q81 |
| 5-246 | A28 | J1 | Q81 |
| 5-247 | A29 | J1 | Q81 |
| 5-248 | A30 | J1 | Q81 |
| 5-249 | A31 | J1 | Q81 |
| 5-250 | A51 | J1 | Q81 |
| 5-251 | A57 | J1 | Q81 |
| 5-252 | A58 | J1 | Q81 |
| 5-253 | A1 | J1 | Q98 |
| 5-254 | A2 | J1 | Q98 |
| 5-255 | A6 | J1 | Q98 |
| 5-256 | A9 | J1 | Q98 |
| 5-257 | A11 | J1 | Q98 |
| 5-258 | A12 | J1 | Q98 |
| 5-259 | A16 | J1 | Q98 |
| 5-260 | A19 | J1 | Q98 |
| 5-261 | A20 | J1 | Q98 |
| 5-262 | A21 | J1 | Q98 |
| 5-263 | A22 | J1 | Q98 |
| 5-264 | A25 | J1 | Q98 |
| 5-265 | A26 | J1 | Q98 |
| 5-266 | A27 | J1 | Q98 |
| 5-267 | A28 | J1 | Q98 |
| 5-268 | A29 | J1 | Q98 |
| 5-269 | A30 | J1 | Q98 |
| 5-270 | A31 | J1 | Q98 |
| 5-271 | A51 | J1 | Q98 |
| 5-272 | A57 | J1 | Q98 |
| 5-273 | A58 | J1 | Q98 |
| 5-274 | A1 | J1 | Q106 |
| 5-275 | A2 | J1 | Q106 |
| 5-276 | A6 | J1 | Q106 |
| 5-277 | A9 | J1 | Q106 |
| 5-278 | A11 | J1 | Q106 |
| 5-279 | A12 | J1 | Q106 |
| 5-280 | A16 | J1 | Q106 |
| 5-281 | A19 | J1 | Q106 |
| 5-282 | A20 | J1 | Q106 |
| 5-283 | A21 | J1 | Q106 |
| 5-284 | A22 | J1 | Q106 |
| 5-285 | A25 | J1 | Q106 |
| 5-286 | A26 | J1 | Q106 |
| 5-287 | A27 | J1 | Q106 |
| 5-288 | A28 | J1 | Q106 |
| 5-289 | A29 | J1 | Q106 |
| 5-290 | A30 | J1 | Q106 |
| 5-291 | A31 | J1 | Q106 |
| 5-292 | A51 | J1 | Q106 |
| 5-293 | A57 | J1 | Q106 |
| 5-294 | A58 | J1 | Q106 |
| 5-295 | A1 | J1 | Q123 |
| 5-296 | A2 | J1 | Q123 |
| 5-297 | A6 | J1 | Q123 |
| 5-298 | A9 | J1 | Q123 |
| 5-299 | A11 | J1 | Q123 |
| 5-300 | A12 | J1 | Q123 |
| 5-301 | A16 | J1 | Q123 |
| 5-302 | A19 | J1 | Q123 |
| 5-303 | A20 | J1 | Q123 |
| 5-304 | A21 | J1 | Q123 |
| 5-305 | A22 | J1 | Q123 |
| 5-306 | A25 | J1 | Q123 |
| 5-307 | A26 | J1 | Q123 |
| 5-308 | A27 | J1 | Q123 |
| 5-309 | A28 | J1 | Q123 |
| 5-310 | A29 | J1 | Q123 |
| 5-311 | A30 | J1 | Q123 |
| 5-312 | A31 | J1 | Q123 |
| 5-313 | A51 | J1 | Q123 |
| 5-314 | A57 | J1 | Q123 |
| 5-315 | A58 | J1 | Q123 |
| 5-316 | A1 | J1 | Q124 |
| 5-317 | A2 | J1 | Q124 |
| 5-318 | A6 | J1 | Q124 |
| 5-319 | A9 | J1 | Q124 |
| 5-320 | A11 | J1 | Q124 |
| 5-321 | A12 | J1 | Q124 |
| 5-322 | A16 | J1 | Q124 |
| 5-323 | A19 | J1 | Q124 |
| 5-324 | A20 | J1 | Q124 |
| 5-325 | A21 | J1 | Q124 |
| 5-326 | A22 | J1 | Q124 |
| 5-327 | A25 | J1 | Q124 |
| 5-328 | A26 | J1 | Q124 |
| 5-329 | A27 | J1 | Q124 |
| 5-330 | A28 | J1 | Q124 |
| 5-331 | A29 | J1 | Q124 |
| 5-332 | A30 | J1 | Q124 |
| 5-333 | A31 | J1 | Q124 |
| 5-334 | A51 | J1 | Q124 |
| 5-335 | A57 | J1 | Q124 |
| 5-336 | A58 | J1 | Q124 |
| 5-337 | A1 | J1 | Q125 |
| 5-338 | A2 | J1 | Q125 |
| 5-339 | A6 | J1 | Q125 |
| 5-340 | A9 | J1 | Q125 |
| 5-341 | A11 | J1 | Q125 |
| 5-342 | A12 | J1 | Q125 |
| 5-343 | A16 | J1 | Q125 |
| 5-344 | A19 | J1 | Q125 |
| 5-345 | A20 | J1 | Q125 |
| 5-346 | A21 | J1 | Q125 |
| 5-347 | A22 | J1 | Q125 |
| 5-348 | A25 | J1 | Q125 |
| 5-349 | A26 | J1 | Q125 |
| 5-350 | A27 | J1 | Q125 |
| 5-351 | A28 | J1 | Q125 |
| 5-352 | A29 | J1 | Q125 |
| 5-353 | A30 | J1 | Q125 |
| 5-354 | A31 | J1 | Q125 |
| 5-355 | A51 | J1 | Q125 |
| 5-356 | A57 | J1 | Q125 |
| 5-357 | A58 | J1 | Q125 |
| 5-358 | A1 | J1 | Q141 |
| 5-359 | A2 | J1 | Q141 |
| 5-360 | A6 | J1 | Q141 |
| 5-361 | A9 | J1 | Q141 |
| 5-362 | A11 | J1 | Q141 |
| 5-363 | A12 | J1 | Q141 |
| 5-364 | A16 | J1 | Q141 |
| 5-365 | A19 | J1 | Q141 |
| 5-366 | A20 | J1 | Q141 |
| 5-367 | A21 | J1 | Q141 |
| 5-368 | A22 | J1 | Q141 |
| 5-369 | A25 | J1 | Q141 |
| 5-370 | A26 | J1 | Q141 |
| 5-371 | A27 | J1 | Q141 |
| 5-372 | A28 | J1 | Q141 |
| 5-373 | A29 | J1 | Q141 |
| 5-374 | A30 | J1 | Q141 |
| 5-375 | A31 | J1 | Q141 |
| 5-376 | A51 | J1 | Q141 |
| 5-377 | A57 | J1 | Q141 |
| 5-378 | A58 | J1 | Q141 |
| 5-379 | A1 | J1 | Q142 |
| 5-380 | A2 | J1 | Q142 |
| 5-381 | A6 | J1 | Q142 |
| 5-382 | A9 | J1 | Q142 |
| 5-383 | A11 | J1 | Q142 |
| 5-384 | A12 | J1 | Q142 |
| 5-385 | A16 | J1 | Q142 |
| 5-386 | A19 | J1 | Q142 |
| 5-387 | A20 | J1 | Q142 |
| 5-388 | A21 | J1 | Q142 |
| 5-389 | A22 | J1 | Q142 |
| 5-390 | A25 | J1 | Q142 |
| 5-391 | A26 | J1 | Q142 |
| 5-392 | A27 | J1 | Q142 |
| 5-393 | A28 | J1 | Q142 |
| 5-394 | A29 | J1 | Q142 |
| 5-395 | A30 | J1 | Q142 |
| 5-396 | A31 | J1 | Q142 |
| 5-397 | A51 | J1 | Q142 |
| 5-398 | A57 | J1 | Q142 |
| 5-399 | A58 | J1 | Q142 |
| 5-400 | A1 | J1 | Q147 |
| 5-401 | A2 | J1 | Q147 |
| 5-402 | A6 | J1 | Q147 |
| 5-403 | A9 | J1 | Q147 |
| 5-404 | A11 | J1 | Q147 |
| 5-405 | A12 | J1 | Q147 |
| 5-406 | A16 | J1 | Q147 |
| 5-407 | A19 | J1 | Q147 |
| 5-408 | A20 | J1 | Q147 |
| 5-409 | A21 | J1 | Q147 |
| 5-410 | A22 | J1 | Q147 |
| 5-411 | A25 | J1 | Q147 |
| 5-412 | A26 | J1 | Q147 |
| 5-413 | A27 | J1 | Q147 |
| 5-414 | A28 | J1 | Q147 |
| 5-415 | A29 | J1 | Q147 |
| 5-416 | A30 | J1 | Q147 |
| 5-417 | A31 | J1 | Q147 |
| 5-418 | A51 | J1 | Q147 |
| 5-419 | A57 | J1 | Q147 |
| 5-420 | A58 | J1 | Q147 |
| 5-421 | A1 | J1 | Q150 |
| 5-422 | A2 | J1 | Q150 |
| 5-423 | A6 | J1 | Q150 |
| 5-424 | A9 | J1 | Q150 |
| 5-425 | A11 | J1 | Q150 |
| 5-426 | A12 | J1 | Q150 |
| 5-427 | A16 | J1 | Q150 |
| 5-428 | A19 | J1 | Q150 |
| 5-429 | A20 | J1 | Q150 |
| 5-430 | A21 | J1 | Q150 |
| 5-431 | A22 | J1 | Q150 |
| 5-432 | A25 | J1 | Q150 |
| 5-433 | A26 | J1 | Q150 |
| 5-434 | A27 | J1 | Q150 |
| 5-435 | A28 | J1 | Q150 |
| 5-436 | A29 | J1 | Q150 |
| 5-437 | A30 | J1 | Q150 |
| 5-438 | A31 | J1 | Q150 |
| 5-439 | A51 | J1 | Q150 |
| 5-440 | A57 | J1 | Q150 |
| 5-441 | A58 | J1 | Q150 |
| 5-442 | A1 | J1 | Q151 |
| 5-443 | A2 | J1 | Q151 |
| 5-444 | A6 | J1 | Q151 |
| 5-445 | A9 | J1 | Q151 |
| 5-446 | A11 | J1 | Q151 |
| 5-447 | A12 | J1 | Q151 |
| 5-448 | A16 | J1 | Q151 |
| 5-449 | A19 | J1 | Q151 |
| 5-450 | A20 | J1 | Q151 |
| 5-451 | A21 | J1 | Q151 |
| 5-452 | A22 | J1 | Q151 |
| 5-453 | A25 | J1 | Q151 |
| 5-454 | A26 | J1 | Q151 |
| 5-455 | A27 | J1 | Q151 |
| 5-456 | A28 | J1 | Q151 |
| 5-457 | A29 | J1 | Q151 |
| 5-458 | A30 | J1 | Q151 |
| 5-459 | A31 | J1 | Q151 |
| 5-460 | A51 | J1 | Q151 |
| 5-461 | A57 | J1 | Q151 |
| 5-462 | A58 | J1 | Q151 |
| 5-463 | A1 | J1 | Q152 |
| 5-464 | A2 | J1 | Q152 |
| 5-465 | A6 | J1 | Q152 |
| 5-466 | A9 | J1 | Q152 |
| 5-467 | A11 | J1 | Q152 |
| 5-468 | A12 | J1 | Q152 |
| 5-469 | A16 | J1 | Q152 |
| 5-470 | A19 | J1 | Q152 |
| 5-471 | A20 | J1 | Q152 |
| 5-472 | A21 | J1 | Q152 |
| 5-473 | A22 | J1 | Q152 |
| 5-474 | A25 | J1 | Q152 |
| 5-475 | A26 | J1 | Q152 |
| 5-476 | A27 | J1 | Q152 |
| 5-477 | A28 | J1 | Q152 |
| 5-478 | A29 | J1 | Q152 |
| 5-479 | A30 | J1 | Q152 |
| 5-480 | A31 | J1 | Q152 |
| 5-481 | A51 | J1 | Q152 |
| 5-482 | A57 | J1 | Q152 |
| 5-483 | A58 | J1 | Q152 |
| 5-484 | A1 | J1 | Q154 |
| 5-485 | A2 | J1 | Q154 |
| 5-486 | A6 | J1 | Q154 |
| 5-487 | A9 | J1 | Q154 |
| 5-488 | A11 | J1 | Q154 |
| 5-489 | A12 | J1 | Q154 |
| 5-490 | A16 | J1 | Q154 |
| 5-491 | A19 | J1 | Q154 |
| 5-492 | A20 | J1 | Q154 |
| 5-493 | A21 | J1 | Q154 |
| 5-494 | A22 | J1 | Q154 |
| 5-495 | A25 | J1 | Q154 |
| 5-496 | A26 | J1 | Q154 |
| 5-497 | A27 | J1 | Q154 |
| 5-498 | A28 | J1 | Q154 |
| 5-499 | A29 | J1 | Q154 |
| 5-500 | A30 | J1 | Q154 |
| 5-501 | A31 | J1 | Q154 |
| 5-502 | A51 | J1 | Q154 |
| 5-503 | A57 | J1 | Q154 |
| 5-504 | A58 | J1 | Q154 |
| 5-505 | A1 | J1 | Q160 |
| 5-506 | A2 | J1 | Q160 |
| 5-507 | A6 | J1 | Q160 |
| 5-508 | A9 | J1 | Q160 |
| 5-509 | A11 | J1 | Q160 |
| 5-510 | A12 | J1 | Q160 |
| 5-511 | A16 | J1 | Q160 |
| 5-512 | A19 | J1 | Q160 |
| 5-513 | A20 | J1 | Q160 |
| 5-514 | A21 | J1 | Q160 |
| 5-515 | A22 | J1 | Q160 |
| 5-516 | A25 | J1 | Q160 |
| 5-517 | A26 | J1 | Q160 |
| 5-518 | A27 | J1 | Q160 |
| 5-519 | A28 | J1 | Q160 |
| 5-520 | A29 | J1 | Q160 |
| 5-521 | A30 | J1 | Q160 |
| 5-522 | A31 | J1 | Q160 |
| 5-523 | A51 | J1 | Q160 |
| 5-524 | A57 | J1 | Q160 |
| 5-525 | A58 | J1 | Q160 |
| 5-526 | A1 | J1 | Q180 |
| 5-527 | A2 | J1 | Q180 |
| 5-528 | A6 | J1 | Q180 |
| 5-529 | A9 | J1 | Q180 |
| 5-530 | A11 | J1 | Q180 |
| 5-531 | A12 | J1 | Q180 |
| 5-532 | A16 | J1 | Q180 |
| 5-533 | A19 | J1 | Q180 |
| 5-534 | A20 | J1 | Q180 |
| 5-535 | A21 | J1 | Q180 |
| 5-536 | A22 | J1 | Q180 |
| 5-537 | A25 | J1 | Q180 |
| 5-538 | A26 | J1 | Q180 |
| 5-539 | A27 | J1 | Q180 |
| 5-540 | A28 | J1 | Q180 |
| 5-541 | A29 | J1 | Q180 |
| 5-542 | A30 | J1 | Q180 |
| 5-543 | A31 | J1 | Q180 |
| 5-544 | A51 | J1 | Q180 |
| 5-545 | A57 | J1 | Q180 |
| 5-546 | A58 | J1 | Q180 |
| 5-547 | A1 | J1 | Q218 |
| 5-548 | A2 | J1 | Q218 |
| 5-549 | A6 | J1 | Q218 |
| 5-550 | A9 | J1 | Q218 |
| 5-551 | A11 | J1 | Q218 |
| 5-552 | A12 | J1 | Q218 |
| 5-553 | A16 | J1 | Q218 |
| 5-554 | A19 | J1 | Q218 |
| 5-555 | A20 | J1 | Q218 |
| 5-556 | A21 | J1 | Q218 |
| 5-557 | A22 | J1 | Q218 |
| 5-558 | A25 | J1 | Q218 |
| 5-559 | A26 | J1 | Q218 |
| 5-560 | A27 | J1 | Q218 |
| 5-561 | A28 | J1 | Q218 |
| 5-562 | A29 | J1 | Q218 |
| 5-563 | A30 | J1 | Q218 |
| 5-564 | A31 | J1 | Q218 |
| 5-565 | A51 | J1 | Q218 |
| 5-566 | A57 | J1 | Q218 |
| 5-567 | A58 | J1 | Q218 |
| 5-568 | A1 | J1 | Q219 |
| 5-569 | A2 | J1 | Q219 |
| 5-570 | A6 | J1 | Q219 |
| 5-571 | A9 | J1 | Q219 |
| 5-572 | A11 | J1 | Q219 |
| 5-573 | A12 | J1 | Q219 |
| 5-574 | A16 | J1 | Q219 |
| 5-575 | A19 | J1 | Q219 |
| 5-576 | A20 | J1 | Q219 |
| 5-577 | A21 | J1 | Q219 |
| 5-578 | A22 | J1 | Q219 |
| 5-579 | A25 | J1 | Q219 |
| 5-580 | A26 | J1 | Q219 |
| 5-581 | A27 | J1 | Q219 |
| 5-582 | A28 | J1 | Q219 |
| 5-583 | A29 | J1 | Q219 |
| 5-584 | A30 | J1 | Q219 |
| 5-585 | A31 | J1 | Q219 |
| 5-586 | A51 | J1 | Q219 |
| 5-587 | A57 | J1 | Q219 |
| 5-588 | A58 | J1 | Q219 |
| 5-589 | A1 | J1 | Q220 |
| 5-590 | A2 | J1 | Q220 |
| 5-591 | A6 | J1 | Q220 |
| 5-592 | A9 | J1 | Q220 |
| 5-593 | A11 | J1 | Q220 |
| 5-594 | A12 | J1 | Q220 |
| 5-595 | A16 | J1 | Q220 |
| 5-596 | A19 | J1 | Q220 |
| 5-597 | A20 | J1 | Q220 |
| 5-598 | A21 | J1 | Q220 |
| 5-599 | A22 | J1 | Q220 |
| 5-600 | A25 | J1 | Q220 |
| 5-601 | A26 | J1 | Q220 |
| 5-602 | A27 | J1 | Q220 |
| 5-603 | A28 | J1 | Q220 |
| 5-604 | A29 | J1 | Q220 |
| 5-605 | A30 | J1 | Q220 |
| 5-606 | A31 | J1 | Q220 |
| 5-607 | A51 | J1 | Q220 |
| 5-608 | A57 | J1 | Q220 |
| 5-609 | A58 | J1 | Q220 |
| 5-610 | A1 | J1 | Q221 |
| 5-611 | A2 | J1 | Q221 |
| 5-612 | A6 | J1 | Q221 |
| 5-613 | A9 | J1 | Q221 |
| 5-614 | A11 | J1 | Q221 |
| 5-615 | A12 | J1 | Q221 |
| 5-616 | A16 | J1 | Q221 |
| 5-617 | A19 | J1 | Q221 |
| 5-618 | A20 | J1 | Q221 |
| 5-619 | A21 | J1 | Q221 |
| 5-620 | A22 | J1 | Q221 |
| 5-621 | A25 | J1 | Q221 |
| 5-622 | A26 | J1 | Q221 |
| 5-623 | A27 | J1 | Q221 |
| 5-624 | A28 | J1 | Q221 |
| 5-625 | A29 | J1 | Q221 |
| 5-626 | A30 | J1 | Q221 |
| 5-627 | A31 | J1 | Q221 |
| 5-628 | A51 | J1 | Q221 |
| 5-629 | A57 | J1 | Q221 |
| 5-630 | A58 | J1 | Q221 |
| 5-631 | A1 | J1 | Q245 |
| 5-632 | A2 | J1 | Q245 |
| 5-633 | A6 | J1 | Q245 |
| 5-634 | A9 | J1 | Q245 |
| 5-635 | A11 | J1 | Q245 |
| 5-636 | A12 | J1 | Q245 |
| 5-637 | A16 | J1 | Q245 |
| 5-638 | A19 | J1 | Q245 |
| 5-639 | A20 | J1 | Q245 |
| 5-640 | A21 | J1 | Q245 |
| 5-641 | A22 | J1 | Q245 |
| 5-642 | A25 | J1 | Q245 |
| 5-643 | A26 | J1 | Q245 |
| 5-644 | A27 | J1 | Q245 |
| 5-645 | A28 | J1 | Q245 |
| 5-646 | A29 | J1 | Q245 |
| 5-647 | A30 | J1 | Q245 |
| 5-648 | A31 | J1 | Q245 |
| 5-649 | A51 | J1 | Q245 |
| 5-650 | A57 | J1 | Q245 |
| 5-651 | A58 | J1 | Q245 |
| 5-652 | A1 | J1 | Q248 |
| 5-653 | A2 | J1 | Q248 |
| 5-654 | A6 | J1 | Q248 |
| 5-655 | A9 | J1 | Q248 |
| 5-656 | A11 | J1 | Q248 |
| 5-657 | A12 | J1 | Q248 |
| 5-658 | A16 | J1 | Q248 |
| 5-659 | A19 | J1 | Q248 |
| 5-660 | A20 | J1 | Q248 |
| 5-661 | A21 | J1 | Q248 |
| 5-662 | A22 | J1 | Q248 |
| 5-663 | A25 | J1 | Q248 |
| 5-664 | A26 | J1 | Q248 |
| 5-665 | A27 | J1 | Q248 |
| 5-666 | A28 | J1 | Q248 |
| 5-667 | A29 | J1 | Q248 |
| 5-668 | A30 | J1 | Q248 |
| 5-669 | A31 | J1 | Q248 |
| 5-670 | A51 | J1 | Q248 |
| 5-671 | A57 | J1 | Q248 |
| 5-672 | A58 | J1 | Q248 |
| 5-673 | A1 | J1 | Q252 |
| 5-674 | A2 | J1 | Q252 |
| 5-675 | A6 | J1 | Q252 |
| 5-676 | A9 | J1 | Q252 |
| 5-677 | A11 | J1 | Q252 |
| 5-678 | A12 | J1 | Q252 |
| 5-679 | A16 | J1 | Q252 |
| 5-680 | A19 | J1 | Q252 |
| 5-681 | A20 | J1 | Q252 |
| 5-682 | A21 | J1 | Q252 |
| 5-683 | A22 | J1 | Q252 |
| 5-684 | A25 | J1 | Q252 |
| 5-685 | A26 | J1 | Q252 |
| 5-686 | A27 | J1 | Q252 |
| 5-687 | A28 | J1 | Q252 |
| 5-688 | A29 | J1 | Q252 |
| 5-689 | A30 | J1 | Q252 |
| 5-690 | A31 | J1 | Q252 |
| 5-691 | A51 | J1 | Q252 |
| 5-692 | A57 | J1 | Q252 |
| 5-693 | A58 | J1 | Q252 |
| 5-694 | A1 | J1 | Q253 |
| 5-695 | A2 | J1 | Q253 |
| 5-696 | A6 | J1 | Q253 |
| 5-697 | A9 | J1 | Q253 |
| 5-698 | A11 | J1 | Q253 |
| 5-699 | A12 | J1 | Q253 |
| 5-700 | A16 | J1 | Q253 |
| 5-701 | A19 | J1 | Q253 |
| 5-702 | A20 | J1 | Q253 |
| 5-703 | A21 | J1 | Q253 |
| 5-704 | A22 | J1 | Q253 |
| 5-705 | A25 | J1 | Q253 |
| 5-706 | A26 | J1 | Q253 |
| 5-707 | A27 | J1 | Q253 |
| 5-708 | A28 | J1 | Q253 |
| 5-709 | A29 | J1 | Q253 |
| 5-710 | A30 | J1 | Q253 |
| 5-711 | A31 | J1 | Q253 |
| 5-712 | A51 | J1 | Q253 |
| 5-713 | A57 | J1 | Q253 |
| 5-714 | A58 | J1 | Q253 |
| 5-715 | A1 | J1 | Q254 |
| 5-716 | A2 | J1 | Q254 |
| 5-717 | A6 | J1 | Q254 |
| 5-718 | A9 | J1 | Q254 |
| 5-719 | A11 | J1 | Q254 |
| 5-720 | A12 | J1 | Q254 |
| 5-721 | A16 | J1 | Q254 |
| 5-722 | A19 | J1 | Q254 |
| 5-723 | A20 | J1 | Q254 |
| 5-724 | A21 | J1 | Q254 |
| 5-725 | A22 | J1 | Q254 |
| 5-726 | A25 | J1 | Q254 |
| 5-727 | A26 | J1 | Q254 |
| 5-728 | A27 | J1 | Q254 |
| 5-729 | A28 | J1 | Q254 |
| 5-730 | A29 | J1 | Q254 |
| 5-731 | A30 | J1 | Q254 |
| 5-732 | A31 | J1 | Q254 |
| 5-733 | A51 | J1 | Q254 |
| 5-734 | A57 | J1 | Q254 |
| 5-735 | A58 | J1 | Q254 |
| 5-736 | A1 | J1 | Q255 |
| 5-737 | A2 | J1 | Q255 |
| 5-738 | A6 | J1 | Q255 |
| 5-739 | A9 | J1 | Q255 |
| 5-740 | A11 | J1 | Q255 |
| 5-741 | A12 | J1 | Q255 |
| 5-742 | A16 | J1 | Q255 |
| 5-743 | A19 | J1 | Q255 |
| 5-744 | A20 | J1 | Q255 |
| 5-745 | A21 | J1 | Q255 |
| 5-746 | A22 | J1 | Q255 |
| 5-747 | A25 | J1 | Q255 |
| 5-748 | A26 | J1 | Q255 |
| 5-749 | A27 | J1 | Q255 |
| 5-750 | A28 | J1 | Q255 |
| 5-751 | A29 | J1 | Q255 |
| 5-752 | A30 | J1 | Q255 |
| 5-753 | A31 | J1 | Q255 |
| 5-754 | A51 | J1 | Q255 |
| 5-755 | A57 | J1 | Q255 |
| 5-756 | A58 | J1 | Q255 |
| 5-757 | A1 | J1 | Q257 |
| 5-758 | A2 | J1 | Q257 |
| 5-759 | A6 | J1 | Q257 |
| 5-760 | A9 | J1 | Q257 |
| 5-761 | A11 | J1 | Q257 |
| 5-762 | A12 | J1 | Q257 |
| 5-763 | A16 | J1 | Q257 |
| 5-764 | A19 | J1 | Q257 |
| 5-765 | A20 | J1 | Q257 |
| 5-766 | A21 | J1 | Q257 |
| 5-767 | A22 | J1 | Q257 |
| 5-768 | A25 | J1 | Q257 |
| 5-769 | A26 | J1 | Q257 |
| 5-770 | A27 | J1 | Q257 |
| 5-771 | A28 | J1 | Q257 |
| 5-772 | A29 | J1 | Q257 |
| 5-773 | A30 | J1 | Q257 |
| 5-774 | A31 | J1 | Q257 |
| 5-775 | A51 | J1 | Q257 |
| 5-776 | A57 | J1 | Q257 |
| 5-777 | A58 | J1 | Q257 |
| 5-778 | A1 | J1 | Q265 |
| 5-779 | A2 | J1 | Q265 |
| 5-780 | A6 | J1 | Q265 |
| 5-781 | A9 | J1 | Q265 |
| 5-782 | A11 | J1 | Q265 |
| 5-783 | A12 | J1 | Q265 |
| 5-784 | A16 | J1 | Q265 |
| 5-785 | A19 | J1 | Q265 |
| 5-786 | A20 | J1 | Q265 |
| 5-787 | A21 | J1 | Q265 |
| 5-788 | A22 | J1 | Q265 |
| 5-789 | A25 | J1 | Q265 |
| 5-790 | A26 | J1 | Q265 |
| 5-791 | A27 | J1 | Q265 |
| 5-792 | A28 | J1 | Q265 |
| 5-793 | A29 | J1 | Q265 |
| 5-794 | A30 | J1 | Q265 |
| 5-795 | A31 | J1 | Q265 |
| 5-796 | A51 | J1 | Q265 |
| 5-797 | A57 | J1 | Q265 |
| 5-798 | A58 | J1 | Q265 |
| 5-799 | A1 | J1 | Q273 |
| 5-800 | A2 | J1 | Q273 |
| 5-801 | A6 | J1 | Q273 |
| 5-802 | A9 | J1 | Q273 |
| 5-803 | A11 | J1 | Q273 |
| 5-804 | A12 | J1 | Q273 |
| 5-805 | A16 | J1 | Q273 |
| 5-806 | A19 | J1 | Q273 |
| 5-807 | A20 | J1 | Q273 |
| 5-808 | A21 | J1 | Q273 |
| 5-809 | A22 | J1 | Q273 |
| 5-810 | A25 | J1 | Q273 |
| 5-811 | A26 | J1 | Q273 |
| 5-812 | A27 | J1 | Q273 |
| 5-813 | A28 | J1 | Q273 |
| 5-814 | A29 | J1 | Q273 |
| 5-815 | A30 | J1 | Q273 |
| 5-816 | A31 | J1 | Q273 |
| 5-817 | A51 | J1 | Q273 |
| 5-818 | A57 | J1 | Q273 |
| 5-819 | A58 | J1 | Q273 |
| 5-820 | A1 | J2 | Q23 |
| 5-821 | A2 | J2 | Q23 |
| 5-822 | A6 | J2 | Q23 |
| 5-823 | A9 | J2 | Q23 |
| 5-824 | A11 | J2 | Q23 |
| 5-825 | A12 | J2 | Q23 |
| 5-826 | A16 | J2 | Q23 |
| 5-827 | A19 | J2 | Q23 |
| 5-828 | A20 | J2 | Q23 |
| 5-829 | A21 | J2 | Q23 |
| 5-830 | A22 | J2 | Q23 |
| 5-831 | A25 | J2 | Q23 |
| 5-832 | A26 | J2 | Q23 |
| 5-833 | A27 | J2 | Q23 |
| 5-834 | A28 | J2 | Q23 |
| 5-835 | A29 | J2 | Q23 |
| 5-836 | A30 | J2 | Q23 |
| 5-837 | A31 | J2 | Q23 |
| 5-838 | A51 | J2 | Q23 |
| 5-839 | A57 | J2 | Q23 |
| 5-840 | A58 | J2 | Q23 |
| 5-841 | A1 | J2 | Q52 |
| 5-842 | A2 | J2 | Q52 |
| 5-843 | A6 | J2 | Q52 |
| 5-844 | A9 | J2 | Q52 |
| 5-845 | A11 | J2 | Q52 |
| 5-846 | A12 | J2 | Q52 |
| 5-847 | A16 | J2 | Q52 |
| 5-848 | A19 | J2 | Q52 |
| 5-849 | A20 | J2 | Q52 |
| 5-850 | A21 | J2 | Q52 |
| 5-851 | A22 | J2 | Q52 |
| 5-852 | A25 | J2 | Q52 |
| 5-853 | A26 | J2 | Q52 |
| 5-854 | A27 | J2 | Q52 |
| 5-855 | A28 | J2 | Q52 |
| 5-856 | A29 | J2 | Q52 |
| 5-857 | A30 | J2 | Q52 |
| 5-858 | A31 | J2 | Q52 |
| 5-859 | A51 | J2 | Q52 |
| 5-860 | A57 | J2 | Q52 |
| 5-861 | A58 | J2 | Q52 |
| 5-862 | A1 | J2 | Q56 |
| 5-863 | A2 | J2 | Q56 |
| 5-864 | A6 | J2 | Q56 |
| 5-865 | A9 | J2 | Q56 |
| 5-866 | A11 | J2 | Q56 |
| 5-867 | A12 | J2 | Q56 |
| 5-868 | A16 | J2 | Q56 |
| 5-869 | A19 | J2 | Q56 |
| 5-870 | A20 | J2 | Q56 |
| 5-871 | A21 | J2 | Q56 |
| 5-872 | A22 | J2 | Q56 |
| 5-873 | A25 | J2 | Q56 |
| 5-874 | A26 | J2 | Q56 |
| 5-875 | A27 | J2 | Q56 |
| 5-876 | A28 | J2 | Q56 |
| 5-877 | A29 | J2 | Q56 |
| 5-878 | A30 | J2 | Q56 |
| 5-879 | A31 | J2 | Q56 |
| 5-880 | A51 | J2 | Q56 |
| 5-881 | A57 | J2 | Q56 |
| 5-882 | A58 | J2 | Q56 |
| 5-883 | A1 | J2 | Q58 |
| 5-884 | A2 | J2 | Q58 |
| 5-885 | A6 | J2 | Q58 |
| 5-886 | A9 | J2 | Q58 |
| 5-887 | A11 | J2 | Q58 |
| 5-888 | A12 | J2 | Q58 |
| 5-889 | A16 | J2 | Q58 |
| 5-890 | A19 | J2 | Q58 |
| 5-891 | A20 | J2 | Q58 |
| 5-892 | A21 | J2 | Q58 |
| 5-893 | A22 | J2 | Q58 |
| 5-894 | A25 | J2 | Q58 |
| 5-895 | A26 | J2 | Q58 |
| 5-896 | A27 | J2 | Q58 |
| 5-897 | A28 | J2 | Q58 |
| 5-898 | A29 | J2 | Q58 |
| 5-899 | A30 | J2 | Q58 |
| 5-900 | A31 | J2 | Q58 |
| 5-901 | A51 | J2 | Q58 |
| 5-902 | A57 | J2 | Q58 |
| 5-903 | A58 | J2 | Q58 |
| 5-904 | A1 | J2 | Q59 |
| 5-905 | A2 | J2 | Q59 |
| 5-906 | A6 | J2 | Q59 |
| 5-907 | A9 | J2 | Q59 |
| 5-908 | A11 | J2 | Q59 |
| 5-909 | A12 | J2 | Q59 |
| 5-910 | A16 | J2 | Q59 |
| 5-911 | A19 | J2 | Q59 |
| 5-912 | A20 | J2 | Q59 |
| 5-913 | A21 | J2 | Q59 |
| 5-914 | A22 | J2 | Q59 |
| 5-915 | A25 | J2 | Q59 |
| 5-916 | A26 | J2 | Q59 |
| 5-917 | A27 | J2 | Q59 |
| 5-918 | A28 | J2 | Q59 |
| 5-919 | A29 | J2 | Q59 |
| 5-920 | A30 | J2 | Q59 |
| 5-921 | A31 | J2 | Q59 |
| 5-922 | A51 | J2 | Q59 |
| 5-923 | A57 | J2 | Q59 |
| 5-924 | A58 | J2 | Q59 |
| 5-925 | A1 | J2 | Q65 |
| 5-926 | A2 | J2 | Q65 |
| 5-927 | A6 | J2 | Q65 |
| 5-928 | A9 | J2 | Q65 |
| 5-929 | A11 | J2 | Q65 |
| 5-930 | A12 | J2 | Q65 |
| 5-931 | A16 | J2 | Q65 |
| 5-932 | A19 | J2 | Q65 |
| 5-933 | A20 | J2 | Q65 |
| 5-934 | A21 | J2 | Q65 |
| 5-935 | A22 | J2 | Q65 |
| 5-936 | A25 | J2 | Q65 |
| 5-937 | A26 | J2 | Q65 |
| 5-938 | A27 | J2 | Q65 |
| 5-939 | A28 | J2 | Q65 |
| 5-940 | A29 | J2 | Q65 |
| 5-941 | A30 | J2 | Q65 |
| 5-942 | A31 | J2 | Q65 |
| 5-943 | A51 | J2 | Q65 |
| 5-944 | A57 | J2 | Q65 |
| 5-945 | A58 | J2 | Q65 |
| 5-946 | A1 | J2 | Q66 |
| 5-947 | A2 | J2 | Q66 |
| 5-948 | A6 | J2 | Q66 |
| 5-949 | A9 | J2 | Q66 |
| 5-950 | A11 | J2 | Q66 |
| 5-951 | A12 | J2 | Q66 |
| 5-952 | A16 | J2 | Q66 |
| 5-953 | A19 | J2 | Q66 |
| 5-954 | A20 | J2 | Q66 |
| 5-955 | A21 | J2 | Q66 |
| 5-956 | A22 | J2 | Q66 |
| 5-957 | A25 | J2 | Q66 |
| 5-958 | A26 | J2 | Q66 |
| 5-959 | A27 | J2 | Q66 |
| 5-960 | A28 | J2 | Q66 |
| 5-961 | A29 | J2 | Q66 |
| 5-962 | A30 | J2 | Q66 |
| 5-963 | A31 | J2 | Q66 |
| 5-964 | A51 | J2 | Q66 |
| 5-965 | A57 | J2 | Q66 |
| 5-966 | A58 | J2 | Q66 |
| 5-967 | A1 | J2 | Q70 |
| 5-968 | A2 | J2 | Q70 |
| 5-969 | A6 | J2 | Q70 |
| 5-970 | A9 | J2 | Q70 |
| 5-971 | A11 | J2 | Q70 |
| 5-972 | A12 | J2 | Q70 |
| 5-973 | A16 | J2 | Q70 |
| 5-974 | A19 | J2 | Q70 |
| 5-975 | A20 | J2 | Q70 |
| 5-976 | A21 | J2 | Q70 |
| 5-977 | A22 | J2 | Q70 |
| 5-978 | A25 | J2 | Q70 |
| 5-979 | A26 | J2 | Q70 |
| 5-980 | A27 | J2 | Q70 |
| 5-981 | A28 | J2 | Q70 |
| 5-982 | A29 | J2 | Q70 |
| 5-983 | A30 | J2 | Q70 |
| 5-984 | A31 | J2 | Q70 |
| 5-985 | A51 | J2 | Q70 |
| 5-986 | A57 | J2 | Q70 |
| 5-987 | A58 | J2 | Q70 |
| 5-988 | A1 | J2 | Q78 |
| 5-989 | A2 | J2 | Q78 |
| 5-990 | A6 | J2 | Q78 |
| 5-991 | A9 | J2 | Q78 |
| 5-992 | A11 | J2 | Q78 |
| 5-993 | A12 | J2 | Q78 |
| 5-994 | A16 | J2 | Q78 |
| 5-995 | A19 | J2 | Q78 |
| 5-996 | A20 | J2 | Q78 |
| 5-997 | A21 | J2 | Q78 |
| 5-998 | A22 | J2 | Q78 |
| 5-999 | A25 | J2 | Q78 |
| 5-1000 | A26 | J2 | Q78 |
| 5-1001 | A27 | J2 | Q78 |
| 5-1002 | A28 | J2 | Q78 |
| 5-1003 | A29 | J2 | Q78 |
| 5-1004 | A30 | J2 | Q78 |
| 5-1005 | A31 | J2 | Q78 |
| 5-1006 | A51 | J2 | Q78 |
| 5-1007 | A57 | J2 | Q78 |
| 5-1008 | A58 | J2 | Q78 |
| 5-1009 | A1 | J2 | Q79 |
| 5-1010 | A2 | J2 | Q79 |
| 5-1011 | A6 | J2 | Q79 |
| 5-1012 | A9 | J2 | Q79 |
| 5-1013 | A11 | J2 | Q79 |
| 5-1014 | A12 | J2 | Q79 |
| 5-1015 | A16 | J2 | Q79 |
| 5-1016 | A19 | J2 | Q79 |
| 5-1017 | A20 | J2 | Q79 |
| 5-1018 | A21 | J2 | Q79 |
| 5-1019 | A22 | J2 | Q79 |
| 5-1020 | A25 | J2 | Q79 |
| 5-1021 | A26 | J2 | Q79 |
| 5-1022 | A27 | J2 | Q79 |
| 5-1023 | A28 | J2 | Q79 |
| 5-1024 | A29 | J2 | Q79 |
| 5-1025 | A30 | J2 | Q79 |
| 5-1026 | A31 | J2 | Q79 |
| 5-1027 | A51 | J2 | Q79 |
| 5-1028 | A57 | J2 | Q79 |
| 5-1029 | A58 | J2 | Q79 |
| 5-1030 | A1 | J2 | Q80 |
| 5-1031 | A2 | J2 | Q80 |
| 5-1032 | A6 | J2 | Q80 |
| 5-1033 | A9 | J2 | Q80 |
| 5-1034 | A11 | J2 | Q80 |
| 5-1035 | A12 | J2 | Q80 |
| 5-1036 | A16 | J2 | Q80 |
| 5-1037 | A19 | J2 | Q80 |
| 5-1038 | A20 | J2 | Q80 |
| 5-1039 | A21 | J2 | Q80 |
| 5-1040 | A22 | J2 | Q80 |
| 5-1041 | A25 | J2 | Q80 |
| 5-1042 | A26 | J2 | Q80 |
| 5-1043 | A27 | J2 | Q80 |
| 5-1044 | A28 | J2 | Q80 |
| 5-1045 | A29 | J2 | Q80 |
| 5-1046 | A30 | J2 | Q80 |
| 5-1047 | A31 | J2 | Q80 |
| 5-1048 | A51 | J2 | Q80 |
| 5-1049 | A57 | J2 | Q80 |
| 5-1050 | A58 | J2 | Q80 |
| 5-1051 | A1 | J2 | Q81 |
| 5-1052 | A2 | J2 | Q81 |
| 5-1053 | A6 | J2 | Q81 |
| 5-1054 | A9 | J2 | Q81 |
| 5-1055 | A11 | J2 | Q81 |
| 5-1056 | A12 | J2 | Q81 |
| 5-1057 | A16 | J2 | Q81 |
| 5-1058 | A19 | J2 | Q81 |
| 5-1059 | A20 | J2 | Q81 |
| 5-1060 | A21 | J2 | Q81 |
| 5-1061 | A22 | J2 | Q81 |
| 5-1062 | A25 | J2 | Q81 |
| 5-1063 | A26 | J2 | Q81 |
| 5-1064 | A27 | J2 | Q81 |
| 5-1065 | A28 | J2 | Q81 |
| 5-1066 | A29 | J2 | Q81 |
| 5-1067 | A30 | J2 | Q81 |
| 5-1068 | A31 | J2 | Q81 |
| 5-1069 | A51 | J2 | Q81 |
| 5-1070 | A57 | J2 | Q81 |
| 5-1071 | A58 | J2 | Q81 |
| 5-1072 | A1 | J2 | Q98 |
| 5-1073 | A2 | J2 | Q98 |
| 5-1074 | A6 | J2 | Q98 |
| 5-1075 | A9 | J2 | Q98 |
| 5-1076 | A11 | J2 | Q98 |
| 5-1077 | A12 | J2 | Q98 |
| 5-1078 | A16 | J2 | Q98 |
| 5-1079 | A19 | J2 | Q98 |
| 5-1080 | A20 | J2 | Q98 |
| 5-1081 | A21 | J2 | Q98 |
| 5-1082 | A22 | J2 | Q98 |
| 5-1083 | A25 | J2 | Q98 |
| 5-1084 | A26 | J2 | Q98 |
| 5-1085 | A27 | J2 | Q98 |
| 5-1086 | A28 | J2 | Q98 |
| 5-1087 | A29 | J2 | Q98 |
| 5-1088 | A30 | J2 | Q98 |
| 5-1089 | A31 | J2 | Q98 |
| 5-1090 | A51 | J2 | Q98 |
| 5-1091 | A57 | J2 | Q98 |
| 5-1092 | A58 | J2 | Q98 |
| 5-1093 | A1 | J2 | Q106 |
| 5-1094 | A2 | J2 | Q106 |
| 5-1095 | A6 | J2 | Q106 |
| 5-1096 | A9 | J2 | Q106 |
| 5-1097 | A11 | J2 | Q106 |
| 5-1098 | A12 | J2 | Q106 |
| 5-1099 | A16 | J2 | Q106 |
| 5-1100 | A19 | J2 | Q106 |
| 5-1101 | A20 | J2 | Q106 |
| 5-1102 | A21 | J2 | Q106 |
| 5-1103 | A22 | J2 | Q106 |
| 5-1104 | A25 | J2 | Q106 |
| 5-1105 | A26 | J2 | Q106 |
| 5-1106 | A27 | J2 | Q106 |
| 5-1107 | A28 | J2 | Q106 |
| 5-1108 | A29 | J2 | Q106 |
| 5-1109 | A30 | J2 | Q106 |
| 5-1110 | A31 | J2 | Q106 |
| 5-1111 | A51 | J2 | Q106 |
| 5-1112 | A57 | J2 | Q106 |
| 5-1113 | A58 | J2 | Q106 |
| 5-1114 | A1 | J2 | Q123 |
| 5-1115 | A2 | J2 | Q123 |
| 5-1116 | A6 | J2 | Q123 |
| 5-1117 | A9 | J2 | Q123 |
| 5-1118 | A11 | J2 | Q123 |
| 5-1119 | A12 | J2 | Q123 |
| 5-1120 | A16 | J2 | Q123 |
| 5-1121 | A19 | J2 | Q123 |
| 5-1122 | A20 | J2 | Q123 |
| 5-1123 | A21 | J2 | Q123 |
| 5-1124 | A22 | J2 | Q123 |
| 5-1125 | A25 | J2 | Q123 |
| 5-1126 | A26 | J2 | Q123 |
| 5-1127 | A27 | J2 | Q123 |
| 5-1128 | A28 | J2 | Q123 |
| 5-1129 | A29 | J2 | Q123 |
| 5-1130 | A30 | J2 | Q123 |
| 5-1131 | A31 | J2 | Q123 |
| 5-1132 | A51 | J2 | Q123 |
| 5-1133 | A57 | J2 | Q123 |
| 5-1134 | A58 | J2 | Q123 |
| 5-1135 | A1 | J2 | Q124 |
| 5-1136 | A2 | J2 | Q124 |
| 5-1137 | A6 | J2 | Q124 |
| 5-1138 | A9 | J2 | Q124 |
| 5-1139 | A11 | J2 | Q124 |
| 5-1140 | A12 | J2 | Q124 |
| 5-1141 | A16 | J2 | Q124 |
| 5-1142 | A19 | J2 | Q124 |
| 5-1143 | A20 | J2 | Q124 |
| 5-1144 | A21 | J2 | Q124 |
| 5-1145 | A22 | J2 | Q124 |
| 5-1146 | A25 | J2 | Q124 |
| 5-1147 | A26 | J2 | Q124 |
| 5-1148 | A27 | J2 | Q124 |
| 5-1149 | A28 | J2 | Q124 |
| 5-1150 | A29 | J2 | Q124 |
| 5-1151 | A30 | J2 | Q124 |
| 5-1152 | A31 | J2 | Q124 |
| 5-1153 | A51 | J2 | Q124 |
| 5-1154 | A57 | J2 | Q124 |
| 5-1155 | A58 | J2 | Q124 |
| 5-1156 | A1 | J2 | Q125 |
| 5-1157 | A2 | J2 | Q125 |
| 5-1158 | A6 | J2 | Q125 |
| 5-1159 | A9 | J2 | Q125 |
| 5-1160 | A11 | J2 | Q125 |
| 5-1161 | A12 | J2 | Q125 |
| 5-1162 | A16 | J2 | Q125 |
| 5-1163 | A19 | J2 | Q125 |
| 5-1164 | A20 | J2 | Q125 |
| 5-1165 | A21 | J2 | Q125 |
| 5-1166 | A22 | J2 | Q125 |
| 5-1167 | A25 | J2 | Q125 |
| 5-1168 | A26 | J2 | Q125 |
| 5-1169 | A27 | J2 | Q125 |
| 5-1170 | A28 | J2 | Q125 |
| 5-1171 | A29 | J2 | Q125 |
| 5-1172 | A30 | J2 | Q125 |
| 5-1173 | A31 | J2 | Q125 |
| 5-1174 | A51 | J2 | Q125 |
| 5-1175 | A57 | J2 | Q125 |
| 5-1176 | A58 | J2 | Q125 |
| 5-1177 | A1 | J2 | Q141 |
| 5-1178 | A2 | J2 | Q141 |
| 5-1179 | A6 | J2 | Q141 |
| 5-1180 | A9 | J2 | Q141 |
| 5-1181 | A11 | J2 | Q141 |
| 5-1182 | A12 | J2 | Q141 |
| 5-1183 | A16 | J2 | Q141 |
| 5-1184 | A19 | J2 | Q141 |
| 5-1185 | A20 | J2 | Q141 |
| 5-1186 | A21 | J2 | Q141 |
| 5-1187 | A22 | J2 | Q141 |
| 5-1188 | A25 | J2 | Q141 |
| 5-1189 | A26 | J2 | Q141 |
| 5-1190 | A27 | J2 | Q141 |
| 5-1191 | A28 | J2 | Q141 |
| 5-1192 | A29 | J2 | Q141 |
| 5-1193 | A30 | J2 | Q141 |
| 5-1194 | A31 | J2 | Q141 |
| 5-1195 | A51 | J2 | Q141 |
| 5-1196 | A57 | J2 | Q141 |
| 5-1197 | A58 | J2 | Q141 |
| 5-1198 | A1 | J2 | Q142 |
| 5-1199 | A2 | J2 | Q142 |
| 5-1200 | A6 | J2 | Q142 |
| 5-1201 | A9 | J2 | Q142 |
| 5-1202 | A11 | J2 | Q142 |
| 5-1203 | A12 | J2 | Q142 |
| 5-1204 | A16 | J2 | Q142 |
| 5-1205 | A19 | J2 | Q142 |
| 5-1206 | A20 | J2 | Q142 |
| 5-1207 | A21 | J2 | Q142 |
| 5-1208 | A22 | J2 | Q142 |
| 5-1209 | A25 | J2 | Q142 |
| 5-1210 | A26 | J2 | Q142 |
| 5-1211 | A27 | J2 | Q142 |
| 5-1212 | A28 | J2 | Q142 |
| 5-1213 | A29 | J2 | Q142 |
| 5-1214 | A30 | J2 | Q142 |
| 5-1215 | A31 | J2 | Q142 |
| 5-1216 | A51 | J2 | Q142 |
| 5-1217 | A57 | J2 | Q142 |
| 5-1218 | A58 | J2 | Q142 |
| 5-1219 | A1 | J2 | Q147 |
| 5-1220 | A2 | J2 | Q147 |
| 5-1221 | A6 | J2 | Q147 |
| 5-1222 | A9 | J2 | Q147 |
| 5-1223 | A11 | J2 | Q147 |
| 5-1224 | A12 | J2 | Q147 |
| 5-1225 | A16 | J2 | Q147 |
| 5-1226 | A19 | J2 | Q147 |
| 5-1227 | A20 | J2 | Q147 |
| 5-1228 | A21 | J2 | Q147 |
| 5-1229 | A22 | J2 | Q147 |
| 5-1230 | A25 | J2 | Q147 |
| 5-1231 | A26 | J2 | Q147 |
| 5-1232 | A27 | J2 | Q147 |
| 5-1233 | A28 | J2 | Q147 |
| 5-1234 | A29 | J2 | Q147 |
| 5-1235 | A30 | J2 | Q147 |
| 5-1236 | A31 | J2 | Q147 |
| 5-1237 | A51 | J2 | Q147 |
| 5-1238 | A57 | J2 | Q147 |
| 5-1239 | A58 | J2 | Q147 |
| 5-1240 | A1 | J2 | Q150 |
| 5-1241 | A2 | J2 | Q150 |
| 5-1242 | A6 | J2 | Q150 |
| 5-1243 | A9 | J2 | Q150 |
| 5-1244 | A11 | J2 | Q150 |
| 5-1245 | A12 | J2 | Q150 |
| 5-1246 | A16 | J2 | Q150 |
| 5-1247 | A19 | J2 | Q150 |
| 5-1248 | A20 | J2 | Q150 |
| 5-1249 | A21 | J2 | Q150 |
| 5-1250 | A22 | J2 | Q150 |
| 5-1251 | A25 | J2 | Q150 |
| 5-1252 | A26 | J2 | Q150 |
| 5-1253 | A27 | J2 | Q150 |
| 5-1254 | A28 | J2 | Q150 |
| 5-1255 | A29 | J2 | Q150 |
| 5-1256 | A30 | J2 | Q150 |
| 5-1257 | A31 | J2 | Q150 |
| 5-1258 | A51 | J2 | Q150 |
| 5-1259 | A57 | J2 | Q150 |
| 5-1260 | A58 | J2 | Q150 |
| 5-1261 | A1 | J2 | Q151 |
| 5-1262 | A2 | J2 | Q151 |
| 5-1263 | A6 | J2 | Q151 |
| 5-1264 | A9 | J2 | Q151 |
| 5-1265 | A11 | J2 | Q151 |
| 5-1266 | A12 | J2 | Q151 |
| 5-1267 | A16 | J2 | Q151 |
| 5-1268 | A19 | J2 | Q151 |
| 5-1269 | A20 | J2 | Q151 |
| 5-1270 | A21 | J2 | Q151 |
| 5-1271 | A22 | J2 | Q151 |
| 5-1272 | A25 | J2 | Q151 |
| 5-1273 | A26 | J2 | Q151 |
| 5-1274 | A27 | J2 | Q151 |
| 5-1275 | A28 | J2 | Q151 |
| 5-1276 | A29 | J2 | Q151 |
| 5-1277 | A30 | J2 | Q151 |
| 5-1278 | A31 | J2 | Q151 |
| 5-1279 | A51 | J2 | Q151 |
| 5-1280 | A57 | J2 | Q151 |
| 5-1281 | A58 | J2 | Q151 |
| 5-1282 | A1 | J2 | Q152 |
| 5-1283 | A2 | J2 | Q152 |
| 5-1284 | A6 | J2 | Q152 |
| 5-1285 | A9 | J2 | Q152 |
| 5-1286 | A11 | J2 | Q152 |
| 5-1287 | A12 | J2 | Q152 |
| 5-1288 | A16 | J2 | Q152 |
| 5-1289 | A19 | J2 | Q152 |
| 5-1290 | A20 | J2 | Q152 |
| 5-1291 | A21 | J2 | Q152 |
| 5-1292 | A22 | J2 | Q152 |
| 5-1293 | A25 | J2 | Q152 |
| 5-1294 | A26 | J2 | Q152 |
| 5-1295 | A27 | J2 | Q152 |
| 5-1296 | A28 | J2 | Q152 |
| 5-1297 | A29 | J2 | Q152 |
| 5-1298 | A30 | J2 | Q152 |
| 5-1299 | A31 | J2 | Q152 |
| 5-1300 | A51 | J2 | Q152 |
| 5-1301 | A57 | J2 | Q152 |
| 5-1302 | A58 | J2 | Q152 |
| 5-1303 | A1 | J2 | Q154 |
| 5-1304 | A2 | J2 | Q154 |
| 5-1305 | A6 | J2 | Q154 |
| 5-1306 | A9 | J2 | Q154 |
| 5-1307 | A11 | J2 | Q154 |
| 5-1308 | A12 | J2 | Q154 |
| 5-1309 | A16 | J2 | Q154 |
| 5-1310 | A19 | J2 | Q154 |
| 5-1311 | A20 | J2 | Q154 |
| 5-1312 | A21 | J2 | Q154 |
| 5-1313 | A22 | J2 | Q154 |
| 5-1314 | A25 | J2 | Q154 |
| 5-1315 | A26 | J2 | Q154 |
| 5-1316 | A27 | J2 | Q154 |
| 5-1317 | A28 | J2 | Q154 |
| 5-1318 | A29 | J2 | Q154 |
| 5-1319 | A30 | J2 | Q154 |
| 5-1320 | A31 | J2 | Q154 |
| 5-1321 | A51 | J2 | Q154 |
| 5-1322 | A57 | J2 | Q154 |
| 5-1323 | A58 | J2 | Q154 |
| 5-1324 | A1 | J2 | Q160 |
| 5-1325 | A2 | J2 | Q160 |
| 5-1326 | A6 | J2 | Q160 |
| 5-1327 | A9 | J2 | Q160 |
| 5-1328 | A11 | J2 | Q160 |
| 5-1329 | A12 | J2 | Q160 |
| 5-1330 | A16 | J2 | Q160 |
| 5-1331 | A19 | J2 | Q160 |
| 5-1332 | A20 | J2 | Q160 |
| 5-1333 | A21 | J2 | Q160 |
| 5-1334 | A22 | J2 | Q160 |
| 5-1335 | A25 | J2 | Q160 |
| 5-1336 | A26 | J2 | Q160 |
| 5-1337 | A27 | J2 | Q160 |
| 5-1338 | A28 | J2 | Q160 |
| 5-1339 | A29 | J2 | Q160 |
| 5-1340 | A30 | J2 | Q160 |
| 5-1341 | A31 | J2 | Q160 |
| 5-1342 | A51 | J2 | Q160 |
| 5-1343 | A57 | J2 | Q160 |
| 5-1344 | A58 | J2 | Q160 |
| 5-1345 | A1 | J2 | Q180 |
| 5-1346 | A2 | J2 | Q180 |
| 5-1347 | A6 | J2 | Q180 |
| 5-1348 | A9 | J2 | Q180 |
| 5-1349 | A11 | J2 | Q180 |
| 5-1350 | A12 | J2 | Q180 |
| 5-1351 | A16 | J2 | Q180 |
| 5-1352 | A19 | J2 | Q180 |
| 5-1353 | A20 | J2 | Q180 |
| 5-1354 | A21 | J2 | Q180 |
| 5-1355 | A22 | J2 | Q180 |
| 5-1356 | A25 | J2 | Q180 |
| 5-1357 | A26 | J2 | Q180 |
| 5-1358 | A27 | J2 | Q180 |
| 5-1359 | A28 | J2 | Q180 |
| 5-1360 | A29 | J2 | Q180 |
| 5-1361 | A30 | J2 | Q180 |
| 5-1362 | A31 | J2 | Q180 |
| 5-1363 | A51 | J2 | Q180 |
| 5-1364 | A57 | J2 | Q180 |
| 5-1365 | A58 | J2 | Q180 |
| 5-1366 | A1 | J2 | Q218 |
| 5-1367 | A2 | J2 | Q218 |
| 5-1368 | A6 | J2 | Q218 |
| 5-1369 | A9 | J2 | Q218 |
| 5-1370 | A11 | J2 | Q218 |
| 5-1371 | A12 | J2 | Q218 |
| 5-1372 | A16 | J2 | Q218 |
| 5-1373 | A19 | J2 | Q218 |
| 5-1374 | A20 | J2 | Q218 |
| 5-1375 | A21 | J2 | Q218 |
| 5-1376 | A22 | J2 | Q218 |
| 5-1377 | A25 | J2 | Q218 |
| 5-1378 | A26 | J2 | Q218 |
| 5-1379 | A27 | J2 | Q218 |
| 5-1380 | A28 | J2 | Q218 |
| 5-1381 | A29 | J2 | Q218 |
| 5-1382 | A30 | J2 | Q218 |
| 5-1383 | A31 | J2 | Q218 |
| 5-1384 | A51 | J2 | Q218 |
| 5-1385 | A57 | J2 | Q218 |
| 5-1386 | A58 | J2 | Q218 |
| 5-1387 | A1 | J2 | Q219 |
| 5-1388 | A2 | J2 | Q219 |
| 5-1389 | A6 | J2 | Q219 |
| 5-1390 | A9 | J2 | Q219 |
| 5-1391 | A11 | J2 | Q219 |
| 5-1392 | A12 | J2 | Q219 |
| 5-1393 | A16 | J2 | Q219 |
| 5-1394 | A19 | J2 | Q219 |
| 5-1395 | A20 | J2 | Q219 |
| 5-1396 | A21 | J2 | Q219 |
| 5-1397 | A22 | J2 | Q219 |
| 5-1398 | A25 | J2 | Q219 |
| 5-1399 | A26 | J2 | Q219 |
| 5-1400 | A27 | J2 | Q219 |
| 5-1401 | A28 | J2 | Q219 |
| 5-1402 | A29 | J2 | Q219 |
| 5-1403 | A30 | J2 | Q219 |
| 5-1404 | A31 | J2 | Q219 |
| 5-1405 | A51 | J2 | Q219 |
| 5-1406 | A57 | J2 | Q219 |
| 5-1407 | A58 | J2 | Q219 |
| 5-1408 | A1 | J2 | Q220 |
| 5-1409 | A2 | J2 | Q220 |
| 5-1410 | A6 | J2 | Q220 |
| 5-1411 | A9 | J2 | Q220 |
| 5-1412 | A11 | J2 | Q220 |
| 5-1413 | A12 | J2 | Q220 |
| 5-1414 | A16 | J2 | Q220 |
| 5-1415 | A19 | J2 | Q220 |
| 5-1416 | A20 | J2 | Q220 |
| 5-1417 | A21 | J2 | Q220 |
| 5-1418 | A22 | J2 | Q220 |
| 5-1419 | A25 | J2 | Q220 |
| 5-1420 | A26 | J2 | Q220 |
| 5-1421 | A27 | J2 | Q220 |
| 5-1422 | A28 | J2 | Q220 |
| 5-1423 | A29 | J2 | Q220 |
| 5-1424 | A30 | J2 | Q220 |
| 5-1425 | A31 | J2 | Q220 |
| 5-1426 | A51 | J2 | Q220 |
| 5-1427 | A57 | J2 | Q220 |
| 5-1428 | A58 | J2 | Q220 |
| 5-1429 | A1 | J2 | Q221 |
| 5-1430 | A2 | J2 | Q221 |
| 5-1431 | A6 | J2 | Q221 |
| 5-1432 | A9 | J2 | Q221 |
| 5-1433 | A11 | J2 | Q221 |
| 5-1434 | A12 | J2 | Q221 |
| 5-1435 | A16 | J2 | Q221 |
| 5-1436 | A19 | J2 | Q221 |
| 5-1437 | A20 | J2 | Q221 |
| 5-1438 | A21 | J2 | Q221 |
| 5-1439 | A22 | J2 | Q221 |
| 5-1440 | A25 | J2 | Q221 |
| 5-1441 | A26 | J2 | Q221 |
| 5-1442 | A27 | J2 | Q221 |
| 5-1443 | A28 | J2 | Q221 |
| 5-1444 | A29 | J2 | Q221 |
| 5-1445 | A30 | J2 | Q221 |
| 5-1446 | A31 | J2 | Q221 |
| 5-1447 | A51 | J2 | Q221 |
| 5-1448 | A57 | J2 | Q221 |
| 5-1449 | A58 | J2 | Q221 |
| 5-1450 | A1 | J2 | Q245 |
| 5-1451 | A2 | J2 | Q245 |
| 5-1452 | A6 | J2 | Q245 |
| 5-1453 | A9 | J2 | Q245 |
| 5-1454 | A11 | J2 | Q245 |
| 5-1455 | A12 | J2 | Q245 |
| 5-1456 | A16 | J2 | Q245 |
| 5-1457 | A19 | J2 | Q245 |
| 5-1458 | A20 | J2 | Q245 |
| 5-1459 | A21 | J2 | Q245 |
| 5-1460 | A22 | J2 | Q245 |
| 5-1461 | A25 | J2 | Q245 |
| 5-1462 | A26 | J2 | Q245 |
| 5-1463 | A27 | J2 | Q245 |
| 5-1464 | A28 | J2 | Q245 |
| 5-1465 | A29 | J2 | Q245 |
| 5-1466 | A30 | J2 | Q245 |
| 5-1467 | A31 | J2 | Q245 |
| 5-1468 | A51 | J2 | Q245 |
| 5-1469 | A57 | J2 | Q245 |
| 5-1470 | A58 | J2 | Q245 |
| 5-1471 | A1 | J2 | Q248 |
| 5-1472 | A2 | J2 | Q248 |
| 5-1473 | A6 | J2 | Q248 |
| 5-1474 | A9 | J2 | Q248 |
| 5-1475 | A11 | J2 | Q248 |
| 5-1476 | A12 | J2 | Q248 |
| 5-1477 | A16 | J2 | Q248 |
| 5-1478 | A19 | J2 | Q248 |
| 5-1479 | A20 | J2 | Q248 |
| 5-1480 | A21 | J2 | Q248 |
| 5-1481 | A22 | J2 | Q248 |
| 5-1482 | A25 | J2 | Q248 |
| 5-1483 | A26 | J2 | Q248 |
| 5-1484 | A27 | J2 | Q248 |
| 5-1485 | A28 | J2 | Q248 |
| 5-1486 | A29 | J2 | Q248 |
| 5-1487 | A30 | J2 | Q248 |
| 5-1488 | A31 | J2 | Q248 |
| 5-1489 | A51 | J2 | Q248 |
| 5-1490 | A57 | J2 | Q248 |
| 5-1491 | A58 | J2 | Q248 |
| 5-1492 | A1 | J2 | Q252 |
| 5-1493 | A2 | J2 | Q252 |
| 5-1494 | A6 | J2 | Q252 |
| 5-1495 | A9 | J2 | Q252 |
| 5-1496 | A11 | J2 | Q252 |
| 5-1497 | A12 | J2 | Q252 |
| 5-1498 | A16 | J2 | Q252 |
| 5-1499 | A19 | J2 | Q252 |
| 5-1500 | A20 | J2 | Q252 |
| 5-1501 | A21 | J2 | Q252 |
| 5-1502 | A22 | J2 | Q252 |
| 5-1503 | A25 | J2 | Q252 |
| 5-1504 | A26 | J2 | Q252 |
| 5-1505 | A27 | J2 | Q252 |
| 5-1506 | A28 | J2 | Q252 |
| 5-1507 | A29 | J2 | Q252 |
| 5-1508 | A30 | J2 | Q252 |
| 5-1509 | A31 | J2 | Q252 |
| 5-1510 | A51 | J2 | Q252 |
| 5-1511 | A57 | J2 | Q252 |
| 5-1512 | A58 | J2 | Q252 |
| 5-1513 | A1 | J2 | Q253 |
| 5-1514 | A2 | J2 | Q253 |
| 5-1515 | A6 | J2 | Q253 |
| 5-1516 | A9 | J2 | Q253 |
| 5-1517 | A11 | J2 | Q253 |
| 5-1518 | A12 | J2 | Q253 |
| 5-1519 | A16 | J2 | Q253 |
| 5-1520 | A19 | J2 | Q253 |
| 5-1521 | A20 | J2 | Q253 |
| 5-1522 | A21 | J2 | Q253 |
| 5-1523 | A22 | J2 | Q253 |
| 5-1524 | A25 | J2 | Q253 |
| 5-1525 | A26 | J2 | Q253 |
| 5-1526 | A27 | J2 | Q253 |
| 5-1527 | A28 | J2 | Q253 |
| 5-1528 | A29 | J2 | Q253 |
| 5-1529 | A30 | J2 | Q253 |
| 5-1530 | A31 | J2 | Q253 |
| 5-1531 | A51 | J2 | Q253 |
| 5-1532 | A57 | J2 | Q253 |
| 5-1533 | A58 | J2 | Q253 |
| 5-1534 | A1 | J2 | Q254 |
| 5-1535 | A2 | J2 | Q254 |
| 5-1536 | A6 | J2 | Q254 |
| 5-1537 | A9 | J2 | Q254 |
| 5-1538 | A11 | J2 | Q254 |
| 5-1539 | A12 | J2 | Q254 |
| 5-1540 | A16 | J2 | Q254 |
| 5-1541 | A19 | J2 | Q254 |
| 5-1542 | A20 | J2 | Q254 |
| 5-1543 | A21 | J2 | Q254 |
| 5-1544 | A22 | J2 | Q254 |
| 5-1545 | A25 | J2 | Q254 |
| 5-1546 | A26 | J2 | Q254 |
| 5-1547 | A27 | J2 | Q254 |
| 5-1548 | A28 | J2 | Q254 |
| 5-1549 | A29 | J2 | Q254 |
| 5-1550 | A30 | J2 | Q254 |
| 5-1551 | A31 | J2 | Q254 |
| 5-1552 | A51 | J2 | Q254 |
| 5-1553 | A57 | J2 | Q254 |
| 5-1554 | A58 | J2 | Q254 |
| 5-1555 | A1 | J2 | Q255 |
| 5-1556 | A2 | J2 | Q255 |
| 5-1557 | A6 | J2 | Q255 |
| 5-1558 | A9 | J2 | Q255 |
| 5-1559 | A11 | J2 | Q255 |
| 5-1560 | A12 | J2 | Q255 |
| 5-1561 | A16 | J2 | Q255 |
| 5-1562 | A19 | J2 | Q255 |
| 5-1563 | A20 | J2 | Q255 |
| 5-1564 | A21 | J2 | Q255 |
| 5-1565 | A22 | J2 | Q255 |
| 5-1566 | A25 | J2 | Q255 |
| 5-1567 | A26 | J2 | Q255 |
| 5-1568 | A27 | J2 | Q255 |
| 5-1569 | A28 | J2 | Q255 |
| 5-1570 | A29 | J2 | Q255 |
| 5-1571 | A30 | J2 | Q255 |
| 5-1572 | A31 | J2 | Q255 |
| 5-1573 | A51 | J2 | Q255 |
| 5-1574 | A57 | J2 | Q255 |
| 5-1575 | A58 | J2 | Q255 |
| 5-1576 | A1 | J2 | Q257 |
| 5-1577 | A2 | J2 | Q257 |
| 5-1578 | A6 | J2 | Q257 |
| 5-1579 | A9 | J2 | Q257 |
| 5-1580 | A11 | J2 | Q257 |
| 5-1581 | A12 | J2 | Q257 |
| 5-1582 | A16 | J2 | Q257 |
| 5-1583 | A19 | J2 | Q257 |
| 5-1584 | A20 | J2 | Q257 |
| 5-1585 | A21 | J2 | Q257 |
| 5-1586 | A22 | J2 | Q257 |
| 5-1587 | A25 | J2 | Q257 |
| 5-1588 | A26 | J2 | Q257 |
| 5-1589 | A27 | J2 | Q257 |
| 5-1590 | A28 | J2 | Q257 |
| 5-1591 | A29 | J2 | Q257 |
| 5-1592 | A30 | J2 | Q257 |
| 5-1593 | A31 | J2 | Q257 |
| 5-1594 | A51 | J2 | Q257 |
| 5-1595 | A57 | J2 | Q257 |
| 5-1596 | A58 | J2 | Q257 |
| 5-1597 | A1 | J2 | Q265 |
| 5-1598 | A2 | J2 | Q265 |
| 5-1599 | A6 | J2 | Q265 |
| 5-1600 | A9 | J2 | Q265 |
| 5-1601 | A11 | J2 | Q265 |
| 5-1602 | A12 | J2 | Q265 |
| 5-1603 | A16 | J2 | Q265 |
| 5-1604 | A19 | J2 | Q265 |
| 5-1605 | A20 | J2 | Q265 |
| 5-1606 | A21 | J2 | Q265 |
| 5-1607 | A22 | J2 | Q265 |
| 5-1608 | A25 | J2 | Q265 |
| 5-1609 | A26 | J2 | Q265 |
| 5-1610 | A27 | J2 | Q265 |
| 5-1611 | A28 | J2 | Q265 |
| 5-1612 | A29 | J2 | Q265 |
| 5-1613 | A30 | J2 | Q265 |
| 5-1614 | A31 | J2 | Q265 |
| 5-1615 | A51 | J2 | Q265 |
| 5-1616 | A57 | J2 | Q265 |
| 5-1617 | A58 | J2 | Q265 |
| 5-1618 | A1 | J2 | Q273 |
| 5-1619 | A2 | J2 | Q273 |
| 5-1620 | A6 | J2 | Q273 |
| 5-1621 | A9 | J2 | Q273 |
| 5-1622 | A11 | J2 | Q273 |
| 5-1623 | A12 | J2 | Q273 |
| 5-1624 | A16 | J2 | Q273 |
| 5-1625 | A19 | J2 | Q273 |
| 5-1626 | A20 | J2 | Q273 |
| 5-1627 | A21 | J2 | Q273 |
| 5-1628 | A22 | J2 | Q273 |
| 5-1629 | A25 | J2 | Q273 |
| 5-1630 | A26 | J2 | Q273 |
| 5-1631 | A27 | J2 | Q273 |
| 5-1632 | A28 | J2 | Q273 |
| 5-1633 | A29 | J2 | Q273 |
| 5-1634 | A30 | J2 | Q273 |
| 5-1635 | A31 | J2 | Q273 |
| 5-1636 | A51 | J2 | Q273 |
| 5-1637 | A57 | J2 | Q273 |
| 5-1638 | A58 | J2 | Q273 |
| 5-1639 | A3 | J2 | Q23 |
| 5-1640 | A4 | J2 | Q23 |
| 5-1641 | A5 | J2 | Q23 |
| 5-1642 | A7 | J2 | Q23 |
| 5-1643 | A8 | J2 | Q23 |
| 5-1644 | A10 | J2 | Q23 |
| 5-1645 | A13 | J2 | Q23 |
| 5-1646 | A14 | J2 | Q23 |
| 5-1647 | A15 | J2 | Q23 |
| 5-1648 | A17 | J2 | Q23 |
| 5-1649 | A18 | J2 | Q23 |
| 5-1650 | A23 | J2 | Q23 |
| 5-1651 | A24 | J2 | Q23 |
| 5-1652 | A32 | J2 | Q23 |
| 5-1653 | A33 | J2 | Q23 |
| 5-1654 | A34 | J2 | Q23 |
| 5-1655 | A35 | J2 | Q23 |
| 5-1656 | A36 | J2 | Q23 |
| 5-1657 | A37 | J2 | Q23 |
| 5-1658 | A38 | J2 | Q23 |
| 5-1659 | A39 | J2 | Q23 |
| 5-1660 | A40 | J2 | Q23 |
| 5-1661 | A41 | J2 | Q23 |
| 5-1662 | A42 | J2 | Q23 |
| 5-1663 | A43 | J2 | Q23 |
| 5-1664 | A44 | J2 | Q23 |
| 5-1665 | A45 | J2 | Q23 |
| 5-1666 | A46 | J2 | Q23 |
| 5-1667 | A47 | J2 | Q23 |
| 5-1668 | A48 | J2 | Q23 |
| 5-1669 | A49 | J2 | Q23 |
| 5-1670 | A50 | J2 | Q23 |
| 5-1671 | A52 | J2 | Q23 |
| 5-1672 | A53 | J2 | Q23 |
| 5-1673 | A54 | J2 | Q23 |
| 5-1674 | A55 | J2 | Q23 |
| 5-1675 | A56 | J2 | Q23 |
| 5-1676 | A59 | J2 | Q23 |
| 5-1677 | A60 | J2 | Q23 |
| 5-1678 | A61 | J2 | Q23 |
| 5-1679 | A62 | J2 | Q23 |
| 5-1680 | A63 | J2 | Q23 |
| 5-1681 | A3 | J2 | Q65 |
| 5-1682 | A4 | J2 | Q65 |
| 5-1683 | A5 | J2 | Q65 |
| 5-1684 | A7 | J2 | Q65 |
| 5-1685 | A8 | J2 | Q65 |
| 5-1686 | A10 | J2 | Q65 |
| 5-1687 | A13 | J2 | Q65 |
| 5-1688 | A14 | J2 | Q65 |
| 5-1689 | A15 | J2 | Q65 |
| 5-1690 | A17 | J2 | Q65 |
| 5-1691 | A18 | J2 | Q65 |
| 5-1692 | A23 | J2 | Q65 |
| 5-1693 | A24 | J2 | Q65 |
| 5-1694 | A32 | J2 | Q65 |
| 5-1695 | A33 | J2 | Q65 |
| 5-1696 | A34 | J2 | Q65 |
| 5-1697 | A35 | J2 | Q65 |
| 5-1698 | A36 | J2 | Q65 |
| 5-1699 | A37 | J2 | Q65 |
| 5-1700 | A38 | J2 | Q65 |
| 5-1701 | A39 | J2 | Q65 |
| 5-1702 | A40 | J2 | Q65 |
| 5-1703 | A41 | J2 | Q65 |
| 5-1704 | A42 | J2 | Q65 |
| 5-1705 | A43 | J2 | Q65 |
| 5-1706 | A44 | J2 | Q65 |
| 5-1707 | A45 | J2 | Q65 |
| 5-1708 | A46 | J2 | Q65 |
| 5-1709 | A47 | J2 | Q65 |
| 5-1710 | A48 | J2 | Q65 |
| 5-1711 | A49 | J2 | Q65 |
| 5-1712 | A50 | J2 | Q65 |
| 5-1713 | A52 | J2 | Q65 |
| 5-1714 | A53 | J2 | Q65 |
| 5-1715 | A54 | J2 | Q65 |
| 5-1716 | A55 | J2 | Q65 |
| 5-1717 | A56 | J2 | Q65 |
| 5-1718 | A59 | J2 | Q65 |
| 5-1719 | A60 | J2 | Q65 |
| 5-1720 | A61 | J2 | Q65 |
| 5-1721 | A62 | J2 | Q65 |
| 5-1722 | A63 | J2 | Q65 |
| 5-1723 | A3 | J2 | Q66 |
| 5-1724 | A4 | J2 | Q66 |
| 5-1725 | A5 | J2 | Q66 |
| 5-1726 | A7 | J2 | Q66 |
| 5-1727 | A8 | J2 | Q66 |
| 5-1728 | A10 | J2 | Q66 |
| 5-1729 | A13 | J2 | Q66 |
| 5-1730 | A14 | J2 | Q66 |
| 5-1731 | A15 | J2 | Q66 |
| 5-1732 | A17 | J2 | Q66 |
| 5-1733 | A18 | J2 | Q66 |
| 5-1734 | A23 | J2 | Q66 |
| 5-1735 | A24 | J2 | Q66 |
| 5-1736 | A32 | J2 | Q66 |
| 5-1737 | A33 | J2 | Q66 |
| 5-1738 | A34 | J2 | Q66 |
| 5-1739 | A35 | J2 | Q66 |
| 5-1740 | A36 | J2 | Q66 |
| 5-1741 | A37 | J2 | Q66 |
| 5-1742 | A38 | J2 | Q66 |
| 5-1743 | A39 | J2 | Q66 |
| 5-1744 | A40 | J2 | Q66 |
| 5-1745 | A41 | J2 | Q66 |
| 5-1746 | A42 | J2 | Q66 |
| 5-1747 | A43 | J2 | Q66 |
| 5-1748 | A44 | J2 | Q66 |
| 5-1749 | A45 | J2 | Q66 |
| 5-1750 | A46 | J2 | Q66 |
| 5-1751 | A47 | J2 | Q66 |
| 5-1752 | A48 | J2 | Q66 |
| 5-1753 | A49 | J2 | Q66 |
| 5-1754 | A50 | J2 | Q66 |
| 5-1755 | A52 | J2 | Q66 |
| 5-1756 | A53 | J2 | Q66 |
| 5-1757 | A54 | J2 | Q66 |
| 5-1758 | A55 | J2 | Q66 |
| 5-1759 | A56 | J2 | Q66 |
| 5-1760 | A59 | J2 | Q66 |
| 5-1761 | A60 | J2 | Q66 |
| 5-1762 | A61 | J2 | Q66 |
| 5-1763 | A62 | J2 | Q66 |
| 5-1764 | A63 | J2 | Q66 |
| 5-1765 | A3 | J2 | Q98 |
| 5-1766 | A4 | J2 | Q98 |
| 5-1767 | A5 | J2 | Q98 |
| 5-1768 | A7 | J2 | Q98 |
| 5-1769 | A8 | J2 | Q98 |
| 5-1770 | A10 | J2 | Q98 |
| 5-1771 | A13 | J2 | Q98 |
| 5-1772 | A14 | J2 | Q98 |
| 5-1773 | A15 | J2 | Q98 |
| 5-1774 | A17 | J2 | Q98 |
| 5-1775 | A18 | J2 | Q98 |
| 5-1776 | A23 | J2 | Q98 |
| 5-1777 | A24 | J2 | Q98 |
| 5-1778 | A32 | J2 | Q98 |
| 5-1779 | A33 | J2 | Q98 |
| 5-1780 | A34 | J2 | Q98 |
| 5-1781 | A35 | J2 | Q98 |
| 5-1782 | A36 | J2 | Q98 |
| 5-1783 | A37 | J2 | Q98 |
| 5-1784 | A38 | J2 | Q98 |
| 5-1785 | A39 | J2 | Q98 |
| 5-1786 | A40 | J2 | Q98 |
| 5-1787 | A41 | J2 | Q98 |
| 5-1788 | A42 | J2 | Q98 |
| 5-1789 | A43 | J2 | Q98 |
| 5-1790 | A44 | J2 | Q98 |
| 5-1791 | A45 | J2 | Q98 |
| 5-1792 | A46 | J2 | Q98 |
| 5-1793 | A47 | J2 | Q98 |
| 5-1794 | A48 | J2 | Q98 |
| 5-1795 | A49 | J2 | Q98 |
| 5-1796 | A50 | J2 | Q98 |
| 5-1797 | A52 | J2 | Q98 |
| 5-1798 | A53 | J2 | Q98 |
| 5-1799 | A54 | J2 | Q98 |
| 5-1800 | A55 | J2 | Q98 |
| 5-1801 | A56 | J2 | Q98 |
| 5-1802 | A59 | J2 | Q98 |
| 5-1803 | A60 | J2 | Q98 |
| 5-1804 | A61 | J2 | Q98 |
| 5-1805 | A62 | J2 | Q98 |
| 5-1806 | A63 | J2 | Q98 |
| 5-1807 | A3 | J2 | Q106 |
| 5-1808 | A4 | J2 | Q106 |
| 5-1809 | A5 | J2 | Q106 |
| 5-1810 | A7 | J2 | Q106 |
| 5-1811 | A8 | J2 | Q106 |
| 5-1812 | A10 | J2 | Q106 |
| 5-1813 | A13 | J2 | Q106 |
| 5-1814 | A14 | J2 | Q106 |
| 5-1815 | A15 | J2 | Q106 |
| 5-1816 | A17 | J2 | Q106 |
| 5-1817 | A18 | J2 | Q106 |
| 5-1818 | A23 | J2 | Q106 |
| 5-1819 | A24 | J2 | Q106 |
| 5-1820 | A32 | J2 | Q106 |
| 5-1821 | A33 | J2 | Q106 |
| 5-1822 | A34 | J2 | Q106 |
| 5-1823 | A35 | J2 | Q106 |
| 5-1824 | A36 | J2 | Q106 |
| 5-1825 | A37 | J2 | Q106 |
| 5-1826 | A38 | J2 | Q106 |
| 5-1827 | A39 | J2 | Q106 |
| 5-1828 | A40 | J2 | Q106 |
| 5-1829 | A41 | J2 | Q106 |
| 5-1830 | A42 | J2 | Q106 |
| 5-1831 | A43 | J2 | Q106 |
| 5-1832 | A44 | J2 | Q106 |
| 5-1833 | A45 | J2 | Q106 |
| 5-1834 | A46 | J2 | Q106 |
| 5-1835 | A47 | J2 | Q106 |
| 5-1836 | A48 | J2 | Q106 |
| 5-1837 | A49 | J2 | Q106 |
| 5-1838 | A50 | J2 | Q106 |
| 5-1839 | A52 | J2 | Q106 |
| 5-1840 | A53 | J2 | Q106 |
| 5-1841 | A54 | J2 | Q106 |
| 5-1842 | A55 | J2 | Q106 |
| 5-1843 | A56 | J2 | Q106 |
| 5-1844 | A59 | J2 | Q106 |
| 5-1845 | A60 | J2 | Q106 |
| 5-1846 | A61 | J2 | Q106 |
| 5-1847 | A62 | J2 | Q106 |
| 5-1848 | A63 | J2 | Q106 |
| 5-1849 | A3 | J2 | Q125 |
| 5-1850 | A4 | J2 | Q125 |
| 5-1851 | A5 | J2 | Q125 |
| 5-1852 | A7 | J2 | Q125 |
| 5-1853 | A8 | J2 | Q125 |
| 5-1854 | A10 | J2 | Q125 |
| 5-1855 | A13 | J2 | Q125 |
| 5-1856 | A14 | J2 | Q125 |
| 5-1857 | A15 | J2 | Q125 |
| 5-1858 | A17 | J2 | Q125 |
| 5-1859 | A18 | J2 | Q125 |
| 5-1860 | A23 | J2 | Q125 |
| 5-1861 | A24 | J2 | Q125 |
| 5-1862 | A32 | J2 | Q125 |
| 5-1863 | A33 | J2 | Q125 |
| 5-1864 | A34 | J2 | Q125 |
| 5-1865 | A35 | J2 | Q125 |
| 5-1866 | A36 | J2 | Q125 |
| 5-1867 | A37 | J2 | Q125 |
| 5-1868 | A38 | J2 | Q125 |
| 5-1869 | A39 | J2 | Q125 |
| 5-1870 | A40 | J2 | Q125 |
| 5-1871 | A41 | J2 | Q125 |
| 5-1872 | A42 | J2 | Q125 |
| 5-1873 | A43 | J2 | Q125 |
| 5-1874 | A44 | J2 | Q125 |
| 5-1875 | A45 | J2 | Q125 |
| 5-1876 | A46 | J2 | Q125 |
| 5-1877 | A47 | J2 | Q125 |
| 5-1878 | A48 | J2 | Q125 |
| 5-1879 | A49 | J2 | Q125 |
| 5-1880 | A50 | J2 | Q125 |
| 5-1881 | A52 | J2 | Q125 |
| 5-1882 | A53 | J2 | Q125 |
| 5-1883 | A54 | J2 | Q125 |
| 5-1884 | A55 | J2 | Q125 |
| 5-1885 | A56 | J2 | Q125 |
| 5-1886 | A59 | J2 | Q125 |
| 5-1887 | A60 | J2 | Q125 |
| 5-1888 | A61 | J2 | Q125 |
| 5-1889 | A62 | J2 | Q125 |
| 5-1890 | A63 | J2 | Q125 |
| 5-1891 | A3 | J2 | Q147 |
| 5-1892 | A4 | J2 | Q147 |
| 5-1893 | A5 | J2 | Q147 |
| 5-1894 | A7 | J2 | Q147 |
| 5-1895 | A8 | J2 | Q147 |
| 5-1896 | A10 | J2 | Q147 |
| 5-1897 | A13 | J2 | Q147 |
| 5-1898 | A14 | J2 | Q147 |
| 5-1899 | A15 | J2 | Q147 |
| 5-1900 | A17 | J2 | Q147 |
| 5-1901 | A18 | J2 | Q147 |
| 5-1902 | A23 | J2 | Q147 |
| 5-1903 | A24 | J2 | Q147 |
| 5-1904 | A32 | J2 | Q147 |
| 5-1905 | A33 | J2 | Q147 |
| 5-1906 | A34 | J2 | Q147 |
| 5-1907 | A35 | J2 | Q147 |
| 5-1908 | A36 | J2 | Q147 |
| 5-1909 | A37 | J2 | Q147 |
| 5-1910 | A38 | J2 | Q147 |
| 5-1911 | A39 | J2 | Q147 |
| 5-1912 | A40 | J2 | Q147 |
| 5-1913 | A41 | J2 | Q147 |
| 5-1914 | A42 | J2 | Q147 |
| 5-1915 | A43 | J2 | Q147 |
| 5-1916 | A44 | J2 | Q147 |
| 5-1917 | A45 | J2 | Q147 |
| 5-1918 | A46 | J2 | Q147 |
| 5-1919 | A47 | J2 | Q147 |
| 5-1920 | A48 | J2 | Q147 |
| 5-1921 | A49 | J2 | Q147 |
| 5-1922 | A50 | J2 | Q147 |
| 5-1923 | A52 | J2 | Q147 |
| 5-1924 | A53 | J2 | Q147 |
| 5-1925 | A54 | J2 | Q147 |
| 5-1926 | A55 | J2 | Q147 |
| 5-1927 | A56 | J2 | Q147 |
| 5-1928 | A59 | J2 | Q147 |
| 5-1929 | A60 | J2 | Q147 |
| 5-1930 | A61 | J2 | Q147 |
| 5-1931 | A62 | J2 | Q147 |
| 5-1932 | A63 | J2 | Q147 |
| 5-1933 | A3 | J2 | Q150 |
| 5-1934 | A4 | J2 | Q150 |
| 5-1935 | A5 | J2 | Q150 |
| 5-1936 | A7 | J2 | Q150 |
| 5-1937 | A8 | J2 | Q150 |
| 5-1938 | A10 | J2 | Q150 |
| 5-1939 | A13 | J2 | Q150 |
| 5-1940 | A14 | J2 | Q150 |
| 5-1941 | A15 | J2 | Q150 |
| 5-1942 | A17 | J2 | Q150 |
| 5-1943 | A18 | J2 | Q150 |
| 5-1944 | A23 | J2 | Q150 |
| 5-1945 | A24 | J2 | Q150 |
| 5-1946 | A32 | J2 | Q150 |
| 5-1947 | A33 | J2 | Q150 |
| 5-1948 | A34 | J2 | Q150 |
| 5-1949 | A35 | J2 | Q150 |
| 5-1950 | A36 | J2 | Q150 |
| 5-1951 | A37 | J2 | Q150 |
| 5-1952 | A38 | J2 | Q150 |
| 5-1953 | A39 | J2 | Q150 |
| 5-1954 | A40 | J2 | Q150 |
| 5-1955 | A41 | J2 | Q150 |
| 5-1956 | A42 | J2 | Q150 |
| 5-1957 | A43 | J2 | Q150 |
| 5-1958 | A44 | J2 | Q150 |
| 5-1959 | A45 | J2 | Q150 |
| 5-1960 | A46 | J2 | Q150 |
| 5-1961 | A47 | J2 | Q150 |
| 5-1962 | A48 | J2 | Q150 |
| 5-1963 | A49 | J2 | Q150 |
| 5-1964 | A50 | J2 | Q150 |
| 5-1965 | A52 | J2 | Q150 |
| 5-1966 | A53 | J2 | Q150 |
| 5-1967 | A54 | J2 | Q150 |
| 5-1968 | A55 | J2 | Q150 |
| 5-1969 | A56 | J2 | Q150 |
| 5-1970 | A59 | J2 | Q150 |
| 5-1971 | A60 | J2 | Q150 |
| 5-1972 | A61 | J2 | Q150 |
| 5-1973 | A62 | J2 | Q150 |
| 5-1974 | A63 | J2 | Q150 |
| 5-1975 | A3 | J2 | Q154 |
| 5-1976 | A4 | J2 | Q154 |
| 5-1977 | A5 | J2 | Q154 |
| 5-1978 | A7 | J2 | Q154 |
| 5-1979 | A8 | J2 | Q154 |
| 5-1980 | A10 | J2 | Q154 |
| 5-1981 | A13 | J2 | Q154 |
| 5-1982 | A14 | J2 | Q154 |
| 5-1983 | A15 | J2 | Q154 |
| 5-1984 | A17 | J2 | Q154 |
| 5-1985 | A18 | J2 | Q154 |
| 5-1986 | A23 | J2 | Q154 |
| 5-1987 | A24 | J2 | Q154 |
| 5-1988 | A32 | J2 | Q154 |
| 5-1989 | A33 | J2 | Q154 |
| 5-1990 | A34 | J2 | Q154 |
| 5-1991 | A35 | J2 | Q154 |
| 5-1992 | A36 | J2 | Q154 |
| 5-1993 | A37 | J2 | Q154 |
| 5-1994 | A38 | J2 | Q154 |
| 5-1995 | A39 | J2 | Q154 |
| 5-1996 | A40 | J2 | Q154 |
| 5-1997 | A41 | J2 | Q154 |
| 5-1998 | A42 | J2 | Q154 |
| 5-1999 | A43 | J2 | Q154 |
| 5-2000 | A44 | J2 | Q154 |
| 5-2001 | A45 | J2 | Q154 |
| 5-2002 | A46 | J2 | Q154 |
| 5-2003 | A47 | J2 | Q154 |
| 5-2004 | A48 | J2 | Q154 |
| 5-2005 | A49 | J2 | Q154 |
| 5-2006 | A50 | J2 | Q154 |
| 5-2007 | A52 | J2 | Q154 |
| 5-2008 | A53 | J2 | Q154 |
| 5-2009 | A54 | J2 | Q154 |
| 5-2010 | A55 | J2 | Q154 |
| 5-2011 | A56 | J2 | Q154 |
| 5-2012 | A59 | J2 | Q154 |
| 5-2013 | A60 | J2 | Q154 |
| 5-2014 | A61 | J2 | Q154 |
| 5-2015 | A62 | J2 | Q154 |
| 5-2016 | A63 | J2 | Q154 |
| 5-2017 | A3 | J2 | Q160 |
| 5-2018 | A4 | J2 | Q160 |
| 5-2019 | A5 | J2 | Q160 |
| 5-2020 | A7 | J2 | Q160 |
| 5-2021 | A8 | J2 | Q160 |
| 5-2022 | A10 | J2 | Q160 |
| 5-2023 | A13 | J2 | Q160 |
| 5-2024 | A14 | J2 | Q160 |
| 5-2025 | A15 | J2 | Q160 |
| 5-2026 | A17 | J2 | Q160 |
| 5-2027 | A18 | J2 | Q160 |
| 5-2028 | A23 | J2 | Q160 |
| 5-2029 | A24 | J2 | Q160 |
| 5-2030 | A32 | J2 | Q160 |
| 5-2031 | A33 | J2 | Q160 |
| 5-2032 | A34 | J2 | Q160 |
| 5-2033 | A35 | J2 | Q160 |
| 5-2034 | A36 | J2 | Q160 |
| 5-2035 | A37 | J2 | Q160 |
| 5-2036 | A38 | J2 | Q160 |
| 5-2037 | A39 | J2 | Q160 |
| 5-2038 | A40 | J2 | Q160 |
| 5-2039 | A41 | J2 | Q160 |
| 5-2040 | A42 | J2 | Q160 |
| 5-2041 | A43 | J2 | Q160 |
| 5-2042 | A44 | J2 | Q160 |
| 5-2043 | A45 | J2 | Q160 |
| 5-2044 | A46 | J2 | Q160 |
| 5-2045 | A47 | J2 | Q160 |
| 5-2046 | A48 | J2 | Q160 |
| 5-2047 | A49 | J2 | Q160 |
| 5-2048 | A50 | J2 | Q160 |
| 5-2049 | A52 | J2 | Q160 |
| 5-2050 | A53 | J2 | Q160 |
| 5-2051 | A54 | J2 | Q160 |
| 5-2052 | A55 | J2 | Q160 |
| 5-2053 | A56 | J2 | Q160 |
| 5-2054 | A59 | J2 | Q160 |
| 5-2055 | A60 | J2 | Q160 |
| 5-2056 | A61 | J2 | Q160 |
| 5-2057 | A62 | J2 | Q160 |
| 5-2058 | A63 | J2 | Q160 |
| 5-2059 | A3 | J2 | Q168 |
| 5-2060 | A4 | J2 | Q168 |
| 5-2061 | A5 | J2 | Q168 |
| 5-2062 | A7 | J2 | Q168 |
| 5-2063 | A8 | J2 | Q168 |
| 5-2064 | A10 | J2 | Q168 |
| 5-2065 | A13 | J2 | Q168 |
| 5-2066 | A14 | J2 | Q168 |
| 5-2067 | A15 | J2 | Q168 |
| 5-2068 | A17 | J2 | Q168 |
| 5-2069 | A18 | J2 | Q168 |
| 5-2070 | A23 | J2 | Q168 |
| 5-2071 | A24 | J2 | Q168 |
| 5-2072 | A32 | J2 | Q168 |
| 5-2073 | A33 | J2 | Q168 |
| 5-2074 | A34 | J2 | Q168 |
| 5-2075 | A35 | J2 | Q168 |
| 5-2076 | A36 | J2 | Q168 |
| 5-2077 | A37 | J2 | Q168 |
| 5-2078 | A38 | J2 | Q168 |
| 5-2079 | A39 | J2 | Q168 |
| 5-2080 | A40 | J2 | Q168 |
| 5-2081 | A41 | J2 | Q168 |
| 5-2082 | A42 | J2 | Q168 |
| 5-2083 | A43 | J2 | Q168 |
| 5-2084 | A44 | J2 | Q168 |
| 5-2085 | A45 | J2 | Q168 |
| 5-2086 | A46 | J2 | Q168 |
| 5-2087 | A47 | J2 | Q168 |
| 5-2088 | A48 | J2 | Q168 |
| 5-2089 | A49 | J2 | Q168 |
| 5-2090 | A50 | J2 | Q168 |
| 5-2091 | A52 | J2 | Q168 |
| 5-2092 | A53 | J2 | Q168 |
| 5-2093 | A54 | J2 | Q168 |
| 5-2094 | A55 | J2 | Q168 |
| 5-2095 | A56 | J2 | Q168 |
| 5-2096 | A59 | J2 | Q168 |
| 5-2097 | A60 | J2 | Q168 |
| 5-2098 | A61 | J2 | Q168 |
| 5-2099 | A62 | J2 | Q168 |
| 5-2100 | A63 | J2 | Q168 |
| 5-2101 | A3 | J2 | Q178 |
| 5-2102 | A4 | J2 | Q178 |
| 5-2103 | A5 | J2 | Q178 |
| 5-2104 | A7 | J2 | Q178 |
| 5-2105 | A8 | J2 | Q178 |
| 5-2106 | A10 | J2 | Q178 |
| 5-2107 | A13 | J2 | Q178 |
| 5-2108 | A14 | J2 | Q178 |
| 5-2109 | A15 | J2 | Q178 |
| 5-2110 | A17 | J2 | Q178 |
| 5-2111 | A18 | J2 | Q178 |
| 5-2112 | A23 | J2 | Q178 |
| 5-2113 | A24 | J2 | Q178 |
| 5-2114 | A32 | J2 | Q178 |
| 5-2115 | A33 | J2 | Q178 |
| 5-2116 | A34 | J2 | Q178 |
| 5-2117 | A35 | J2 | Q178 |
| 5-2118 | A36 | J2 | Q178 |
| 5-2119 | A37 | J2 | Q178 |
| 5-2120 | A38 | J2 | Q178 |
| 5-2121 | A39 | J2 | Q178 |
| 5-2122 | A40 | J2 | Q178 |
| 5-2123 | A41 | J2 | Q178 |
| 5-2124 | A42 | J2 | Q178 |
| 5-2125 | A43 | J2 | Q178 |
| 5-2126 | A44 | J2 | Q178 |
| 5-2127 | A45 | J2 | Q178 |
| 5-2128 | A46 | J2 | Q178 |
| 5-2129 | A47 | J2 | Q178 |
| 5-2130 | A48 | J2 | Q178 |
| 5-2131 | A49 | J2 | Q178 |
| 5-2132 | A50 | J2 | Q178 |
| 5-2133 | A52 | J2 | Q178 |
| 5-2134 | A53 | J2 | Q178 |
| 5-2135 | A54 | J2 | Q178 |
| 5-2136 | A55 | J2 | Q178 |
| 5-2137 | A56 | J2 | Q178 |
| 5-2138 | A59 | J2 | Q178 |
| 5-2139 | A60 | J2 | Q178 |
| 5-2140 | A61 | J2 | Q178 |
| 5-2141 | A62 | J2 | Q178 |
| 5-2142 | A63 | J2 | Q178 |
| 5-2143 | A3 | J2 | Q221 |
| 5-2144 | A4 | J2 | Q221 |
| 5-2145 | A5 | J2 | Q221 |
| 5-2146 | A7 | J2 | Q221 |
| 5-2147 | A8 | J2 | Q221 |
| 5-2148 | A10 | J2 | Q221 |
| 5-2149 | A13 | J2 | Q221 |
| 5-2150 | A14 | J2 | Q221 |
| 5-2151 | A15 | J2 | Q221 |
| 5-2152 | A17 | J2 | Q221 |
| 5-2153 | A18 | J2 | Q221 |
| 5-2154 | A23 | J2 | Q221 |
| 5-2155 | A24 | J2 | Q221 |
| 5-2156 | A32 | J2 | Q221 |
| 5-2157 | A33 | J2 | Q221 |
| 5-2158 | A34 | J2 | Q221 |
| 5-2159 | A35 | J2 | Q221 |
| 5-2160 | A36 | J2 | Q221 |
| 5-2161 | A37 | J2 | Q221 |
| 5-2162 | A38 | J2 | Q221 |
| 5-2163 | A39 | J2 | Q221 |
| 5-2164 | A40 | J2 | Q221 |
| 5-2165 | A41 | J2 | Q221 |
| 5-2166 | A42 | J2 | Q221 |
| 5-2167 | A43 | J2 | Q221 |
| 5-2168 | A44 | J2 | Q221 |
| 5-2169 | A45 | J2 | Q221 |
| 5-2170 | A46 | J2 | Q221 |
| 5-2171 | A47 | J2 | Q221 |
| 5-2172 | A48 | J2 | Q221 |
| 5-2173 | A49 | J2 | Q221 |
| 5-2174 | A50 | J2 | Q221 |
| 5-2175 | A52 | J2 | Q221 |
| 5-2176 | A53 | J2 | Q221 |
| 5-2177 | A54 | J2 | Q221 |
| 5-2178 | A55 | J2 | Q221 |
| 5-2179 | A56 | J2 | Q221 |
| 5-2180 | A59 | J2 | Q221 |
| 5-2181 | A60 | J2 | Q221 |
| 5-2182 | A61 | J2 | Q221 |
| 5-2183 | A62 | J2 | Q221 |
| 5-2184 | A63 | J2 | Q221 |
| 5-2185 | A3 | J1 | Q23 |
| 5-2186 | A4 | J1 | Q23 |
| 5-2187 | A5 | J1 | Q23 |
| 5-2188 | A7 | J1 | Q23 |
| 5-2189 | A8 | J1 | Q23 |
| 5-2190 | A10 | J1 | Q23 |
| 5-2191 | A13 | J1 | Q23 |
| 5-2192 | A14 | J1 | Q23 |
| 5-2193 | A15 | J1 | Q23 |
| 5-2194 | A17 | J1 | Q23 |
| 5-2195 | A18 | J1 | Q23 |
| 5-2196 | A23 | J1 | Q23 |
| 5-2197 | A24 | J1 | Q23 |
| 5-2198 | A32 | J1 | Q23 |
| 5-2199 | A33 | J1 | Q23 |
| 5-2200 | A34 | J1 | Q23 |
| 5-2201 | A35 | J1 | Q23 |
| 5-2202 | A36 | J1 | Q23 |
| 5-2203 | A37 | J1 | Q23 |
| 5-2204 | A38 | J1 | Q23 |
| 5-2205 | A39 | J1 | Q23 |
| 5-2206 | A40 | J1 | Q23 |
| 5-2207 | A41 | J1 | Q23 |
| 5-2208 | A42 | J1 | Q23 |
| 5-2209 | A43 | J1 | Q23 |
| 5-2210 | A44 | J1 | Q23 |
| 5-2211 | A45 | J1 | Q23 |
| 5-2212 | A46 | J1 | Q23 |
| 5-2213 | A47 | J1 | Q23 |
| 5-2214 | A48 | J1 | Q23 |
| 5-2215 | A49 | J1 | Q23 |
| 5-2216 | A50 | J1 | Q23 |
| 5-2217 | A52 | J1 | Q23 |
| 5-2218 | A53 | J1 | Q23 |
| 5-2219 | A54 | J1 | Q23 |
| 5-2220 | A55 | J1 | Q23 |
| 5-2221 | A56 | J1 | Q23 |
| 5-2222 | A59 | J1 | Q23 |
| 5-2223 | A60 | J1 | Q23 |
| 5-2224 | A61 | J1 | Q23 |
| 5-2225 | A62 | J1 | Q23 |
| 5-2226 | A63 | J1 | Q23 |
| 5-2227 | A3 | J1 | Q65 |
| 5-2228 | A4 | J1 | Q65 |
| 5-2229 | A5 | J1 | Q65 |
| 5-2230 | A7 | J1 | Q65 |
| 5-2231 | A8 | J1 | Q65 |
| 5-2232 | A10 | J1 | Q65 |
| 5-2233 | A13 | J1 | Q65 |
| 5-2234 | A14 | J1 | Q65 |
| 5-2235 | A15 | J1 | Q65 |
| 5-2236 | A17 | J1 | Q65 |
| 5-2237 | A18 | J1 | Q65 |
| 5-2238 | A23 | J1 | Q65 |
| 5-2239 | A24 | J1 | Q65 |
| 5-2240 | A32 | J1 | Q65 |
| 5-2241 | A33 | J1 | Q65 |
| 5-2242 | A34 | J1 | Q65 |
| 5-2243 | A35 | J1 | Q65 |
| 5-2244 | A36 | J1 | Q65 |
| 5-2245 | A37 | J1 | Q65 |
| 5-2246 | A38 | J1 | Q65 |
| 5-2247 | A39 | J1 | Q65 |
| 5-2248 | A40 | J1 | Q65 |
| 5-2249 | A41 | J1 | Q65 |
| 5-2250 | A42 | J1 | Q65 |
| 5-2251 | A43 | J1 | Q65 |
| 5-2252 | A44 | J1 | Q65 |
| 5-2253 | A45 | J1 | Q65 |
| 5-2254 | A46 | J1 | Q65 |
| 5-2255 | A47 | J1 | Q65 |
| 5-2256 | A48 | J1 | Q65 |
| 5-2257 | A49 | J1 | Q65 |
| 5-2258 | A50 | J1 | Q65 |
| 5-2259 | A52 | J1 | Q65 |
| 5-2260 | A53 | J1 | Q65 |
| 5-2261 | A54 | J1 | Q65 |
| 5-2262 | A55 | J1 | Q65 |
| 5-2263 | A56 | J1 | Q65 |
| 5-2264 | A59 | J1 | Q65 |
| 5-2265 | A60 | J1 | Q65 |
| 5-2266 | A61 | J1 | Q65 |
| 5-2267 | A62 | J1 | Q65 |
| 5-2268 | A63 | J1 | Q65 |
| 5-2269 | A3 | J1 | Q66 |
| 5-2270 | A4 | J1 | Q66 |
| 5-2271 | A5 | J1 | Q66 |
| 5-2272 | A7 | J1 | Q66 |
| 5-2273 | A8 | J1 | Q66 |
| 5-2274 | A10 | J1 | Q66 |
| 5-2275 | A13 | J1 | Q66 |
| 5-2276 | A14 | J1 | Q66 |
| 5-2277 | A15 | J1 | Q66 |
| 5-2278 | A17 | J1 | Q66 |
| 5-2279 | A18 | J1 | Q66 |
| 5-2280 | A23 | J1 | Q66 |
| 5-2281 | A24 | J1 | Q66 |
| 5-2282 | A32 | J1 | Q66 |
| 5-2283 | A33 | J1 | Q66 |
| 5-2284 | A34 | J1 | Q66 |
| 5-2285 | A35 | J1 | Q66 |
| 5-2286 | A36 | J1 | Q66 |
| 5-2287 | A37 | J1 | Q66 |
| 5-2288 | A38 | J1 | Q66 |
| 5-2289 | A39 | J1 | Q66 |
| 5-2290 | A40 | J1 | Q66 |
| 5-2291 | A41 | J1 | Q66 |
| 5-2292 | A42 | J1 | Q66 |
| 5-2293 | A43 | J1 | Q66 |
| 5-2294 | A44 | J1 | Q66 |
| 5-2295 | A45 | J1 | Q66 |
| 5-2296 | A46 | J1 | Q66 |
| 5-2297 | A47 | J1 | Q66 |
| 5-2298 | A48 | J1 | Q66 |
| 5-2299 | A49 | J1 | Q66 |
| 5-2300 | A50 | J1 | Q66 |
| 5-2301 | A52 | J1 | Q66 |
| 5-2302 | A53 | J1 | Q66 |
| 5-2303 | A54 | J1 | Q66 |
| 5-2304 | A55 | J1 | Q66 |
| 5-2305 | A56 | J1 | Q66 |
| 5-2306 | A59 | J1 | Q66 |
| 5-2307 | A60 | J1 | Q66 |
| 5-2308 | A61 | J1 | Q66 |
| 5-2309 | A62 | J1 | Q66 |
| 5-2310 | A63 | J1 | Q66 |
| 5-2311 | A3 | J1 | Q98 |
| 5-2312 | A4 | J1 | Q98 |
| 5-2313 | A5 | J1 | Q98 |
| 5-2314 | A7 | J1 | Q98 |
| 5-2315 | A8 | J1 | Q98 |
| 5-2316 | A10 | J1 | Q98 |
| 5-2317 | A13 | J1 | Q98 |
| 5-2318 | A14 | J1 | Q98 |
| 5-2319 | A15 | J1 | Q98 |
| 5-2320 | A17 | J1 | Q98 |
| 5-2321 | A18 | J1 | Q98 |
| 5-2322 | A23 | J1 | Q98 |
| 5-2323 | A24 | J1 | Q98 |
| 5-2324 | A32 | J1 | Q98 |
| 5-2325 | A33 | J1 | Q98 |
| 5-2326 | A34 | J1 | Q98 |
| 5-2327 | A35 | J1 | Q98 |
| 5-2328 | A36 | J1 | Q98 |
| 5-2329 | A37 | J1 | Q98 |
| 5-2330 | A38 | J1 | Q98 |
| 5-2331 | A39 | J1 | Q98 |
| 5-2332 | A40 | J1 | Q98 |
| 5-2333 | A41 | J1 | Q98 |
| 5-2334 | A42 | J1 | Q98 |
| 5-2335 | A43 | J1 | Q98 |
| 5-2336 | A44 | J1 | Q98 |
| 5-2337 | A45 | J1 | Q98 |
| 5-2338 | A46 | J1 | Q98 |
| 5-2339 | A47 | J1 | Q98 |
| 5-2340 | A48 | J1 | Q98 |
| 5-2341 | A49 | J1 | Q98 |
| 5-2342 | A50 | J1 | Q98 |
| 5-2343 | A52 | J1 | Q98 |
| 5-2344 | A53 | J1 | Q98 |
| 5-2345 | A54 | J1 | Q98 |
| 5-2346 | A55 | J1 | Q98 |
| 5-2347 | A56 | J1 | Q98 |
| 5-2348 | A59 | J1 | Q98 |
| 5-2349 | A60 | J1 | Q98 |
| 5-2350 | A61 | J1 | Q98 |
| 5-2351 | A62 | J1 | Q98 |
| 5-2352 | A63 | J1 | Q98 |
| 5-2353 | A3 | J1 | Q106 |
| 5-2354 | A4 | J1 | Q106 |
| 5-2355 | A5 | J1 | Q106 |
| 5-2356 | A7 | J1 | Q106 |
| 5-2357 | A8 | J1 | Q106 |
| 5-2358 | A10 | J1 | Q106 |
| 5-2359 | A13 | J1 | Q106 |
| 5-2360 | A14 | J1 | Q106 |
| 5-2361 | A15 | J1 | Q106 |
| 5-2362 | A17 | J1 | Q106 |
| 5-2363 | A18 | J1 | Q106 |
| 5-2364 | A23 | J1 | Q106 |
| 5-2365 | A24 | J1 | Q106 |
| 5-2366 | A32 | J1 | Q106 |
| 5-2367 | A33 | J1 | Q106 |
| 5-2368 | A34 | J1 | Q106 |
| 5-2369 | A35 | J1 | Q106 |
| 5-2370 | A36 | J1 | Q106 |
| 5-2371 | A37 | J1 | Q106 |
| 5-2372 | A38 | J1 | Q106 |
| 5-2373 | A39 | J1 | Q106 |
| 5-2374 | A40 | J1 | Q106 |
| 5-2375 | A41 | J1 | Q106 |
| 5-2376 | A42 | J1 | Q106 |
| 5-2377 | A43 | J1 | Q106 |
| 5-2378 | A44 | J1 | Q106 |
| 5-2379 | A45 | J1 | Q106 |
| 5-2380 | A46 | J1 | Q106 |
| 5-2381 | A47 | J1 | Q106 |
| 5-2382 | A48 | J1 | Q106 |
| 5-2383 | A49 | J1 | Q106 |
| 5-2384 | A50 | J1 | Q106 |
| 5-2385 | A52 | J1 | Q106 |
| 5-2386 | A53 | J1 | Q106 |
| 5-2387 | A54 | J1 | Q106 |
| 5-2388 | A55 | J1 | Q106 |
| 5-2389 | A56 | J1 | Q106 |
| 5-2390 | A59 | J1 | Q106 |
| 5-2391 | A60 | J1 | Q106 |
| 5-2392 | A61 | J1 | Q106 |
| 5-2393 | A62 | J1 | Q106 |
| 5-2394 | A63 | J1 | Q106 |
| 5-2395 | A3 | J1 | Q125 |
| 5-2396 | A4 | J1 | Q125 |
| 5-2397 | A5 | J1 | Q125 |
| 5-2398 | A7 | J1 | Q125 |
| 5-2399 | A8 | J1 | Q125 |
| 5-2400 | A10 | J1 | Q125 |
| 5-2401 | A13 | J1 | Q125 |
| 5-2402 | A14 | J1 | Q125 |
| 5-2403 | A15 | J1 | Q125 |
| 5-2404 | A17 | J1 | Q125 |
| 5-2405 | A18 | J1 | Q125 |
| 5-2406 | A23 | J1 | Q125 |
| 5-2407 | A24 | J1 | Q125 |
| 5-2408 | A32 | J1 | Q125 |
| 5-2409 | A33 | J1 | Q125 |
| 5-2410 | A34 | J1 | Q125 |
| 5-2411 | A35 | J1 | Q125 |
| 5-2412 | A36 | J1 | Q125 |
| 5-2413 | A37 | J1 | Q125 |
| 5-2414 | A38 | J1 | Q125 |
| 5-2415 | A39 | J1 | Q125 |
| 5-2416 | A40 | J1 | Q125 |
| 5-2417 | A41 | J1 | Q125 |
| 5-2418 | A42 | J1 | Q125 |
| 5-2419 | A43 | J1 | Q125 |
| 5-2420 | A44 | J1 | Q125 |
| 5-2421 | A45 | J1 | Q125 |
| 5-2422 | A46 | J1 | Q125 |
| 5-2423 | A47 | J1 | Q125 |
| 5-2424 | A48 | J1 | Q125 |
| 5-2425 | A49 | J1 | Q125 |
| 5-2426 | A50 | J1 | Q125 |
| 5-2427 | A52 | J1 | Q125 |
| 5-2428 | A53 | J1 | Q125 |
| 5-2429 | A54 | J1 | Q125 |
| 5-2430 | A55 | J1 | Q125 |
| 5-2431 | A56 | J1 | Q125 |
| 5-2432 | A59 | J1 | Q125 |
| 5-2433 | A60 | J1 | Q125 |
| 5-2434 | A61 | J1 | Q125 |
| 5-2435 | A62 | J1 | Q125 |
| 5-2436 | A63 | J1 | Q125 |
| 5-2437 | A3 | J1 | Q147 |
| 5-2438 | A4 | J1 | Q147 |
| 5-2439 | A5 | J1 | Q147 |
| 5-2440 | A7 | J1 | Q147 |
| 5-2441 | A8 | J1 | Q147 |
| 5-2442 | A10 | J1 | Q147 |
| 5-2443 | A13 | J1 | Q147 |
| 5-2444 | A14 | J1 | Q147 |
| 5-2445 | A15 | J1 | Q147 |
| 5-2446 | A17 | J1 | Q147 |
| 5-2447 | A18 | J1 | Q147 |
| 5-2448 | A23 | J1 | Q147 |
| 5-2449 | A24 | J1 | Q147 |
| 5-2450 | A32 | J1 | Q147 |
| 5-2451 | A33 | J1 | Q147 |
| 5-2452 | A34 | J1 | Q147 |
| 5-2453 | A35 | J1 | Q147 |
| 5-2454 | A36 | J1 | Q147 |
| 5-2455 | A37 | J1 | Q147 |
| 5-2456 | A38 | J1 | Q147 |
| 5-2457 | A39 | J1 | Q147 |
| 5-2458 | A40 | J1 | Q147 |
| 5-2459 | A41 | J1 | Q147 |
| 5-2460 | A42 | J1 | Q147 |
| 5-2461 | A43 | J1 | Q147 |
| 5-2462 | A44 | J1 | Q147 |
| 5-2463 | A45 | J1 | Q147 |
| 5-2464 | A46 | J1 | Q147 |
| 5-2465 | A47 | J1 | Q147 |
| 5-2466 | A48 | J1 | Q147 |
| 5-2467 | A49 | J1 | Q147 |
| 5-2468 | A50 | J1 | Q147 |
| 5-2469 | A52 | J1 | Q147 |
| 5-2470 | A53 | J1 | Q147 |
| 5-2471 | A54 | J1 | Q147 |
| 5-2472 | A55 | J1 | Q147 |
| 5-2473 | A56 | J1 | Q147 |
| 5-2474 | A59 | J1 | Q147 |
| 5-2475 | A60 | J1 | Q147 |
| 5-2476 | A61 | J1 | Q147 |
| 5-2477 | A62 | J1 | Q147 |
| 5-2478 | A63 | J1 | Q147 |
| 5-2479 | A3 | J1 | Q150 |
| 5-2480 | A4 | J1 | Q150 |
| 5-2481 | A5 | J1 | Q150 |
| 5-2482 | A7 | J1 | Q150 |
| 5-2483 | A8 | J1 | Q150 |
| 5-2484 | A10 | J1 | Q150 |
| 5-2485 | A13 | J1 | Q150 |
| 5-2486 | A14 | J1 | Q150 |
| 5-2487 | A15 | J1 | Q150 |
| 5-2488 | A17 | J1 | Q150 |
| 5-2489 | A18 | J1 | Q150 |
| 5-2490 | A23 | J1 | Q150 |
| 5-2491 | A24 | J1 | Q150 |
| 5-2492 | A32 | J1 | Q150 |
| 5-2493 | A33 | J1 | Q150 |
| 5-2494 | A34 | J1 | Q150 |
| 5-2495 | A35 | J1 | Q150 |
| 5-2496 | A36 | J1 | Q150 |
| 5-2497 | A37 | J1 | Q150 |
| 5-2498 | A38 | J1 | Q150 |
| 5-2499 | A39 | J1 | Q150 |
| 5-2500 | A40 | J1 | Q150 |
| 5-2501 | A41 | J1 | Q150 |
| 5-2502 | A42 | J1 | Q150 |
| 5-2503 | A43 | J1 | Q150 |
| 5-2504 | A44 | J1 | Q150 |
| 5-2505 | A45 | J1 | Q150 |
| 5-2506 | A46 | J1 | Q150 |
| 5-2507 | A47 | J1 | Q150 |
| 5-2508 | A48 | J1 | Q150 |
| 5-2509 | A49 | J1 | Q150 |
| 5-2510 | A50 | J1 | Q150 |
| 5-2511 | A52 | J1 | Q150 |
| 5-2512 | A53 | J1 | Q150 |
| 5-2513 | A54 | J1 | Q150 |
| 5-2514 | A55 | J1 | Q150 |
| 5-2515 | A56 | J1 | Q150 |
| 5-2516 | A59 | J1 | Q150 |
| 5-2517 | A60 | J1 | Q150 |
| 5-2518 | A61 | J1 | Q150 |
| 5-2519 | A62 | J1 | Q150 |
| 5-2520 | A63 | J1 | Q150 |
| 5-2521 | A3 | J1 | Q154 |
| 5-2522 | A4 | J1 | Q154 |
| 5-2523 | A5 | J1 | Q154 |
| 5-2524 | A7 | J1 | Q154 |
| 5-2525 | A8 | J1 | Q154 |
| 5-2526 | A10 | J1 | Q154 |
| 5-2527 | A13 | J1 | Q154 |
| 5-2528 | A14 | J1 | Q154 |
| 5-2529 | A15 | J1 | Q154 |
| 5-2530 | A17 | J1 | Q154 |
| 5-2531 | A18 | J1 | Q154 |
| 5-2532 | A23 | J1 | Q154 |
| 5-2533 | A24 | J1 | Q154 |
| 5-2534 | A32 | J1 | Q154 |
| 5-2535 | A33 | J1 | Q154 |
| 5-2536 | A34 | J1 | Q154 |
| 5-2537 | A35 | J1 | Q154 |
| 5-2538 | A36 | J1 | Q154 |
| 5-2539 | A37 | J1 | Q154 |
| 5-2540 | A38 | J1 | Q154 |
| 5-2541 | A39 | J1 | Q154 |
| 5-2542 | A40 | J1 | Q154 |
| 5-2543 | A41 | J1 | Q154 |
| 5-2544 | A42 | J1 | Q154 |
| 5-2545 | A43 | J1 | Q154 |
| 5-2546 | A44 | J1 | Q154 |
| 5-2547 | A45 | J1 | Q154 |
| 5-2548 | A46 | J1 | Q154 |
| 5-2549 | A47 | J1 | Q154 |
| 5-2550 | A48 | J1 | Q154 |
| 5-2551 | A49 | J1 | Q154 |
| 5-2552 | A50 | J1 | Q154 |
| 5-2553 | A52 | J1 | Q154 |
| 5-2554 | A53 | J1 | Q154 |
| 5-2555 | A54 | J1 | Q154 |
| 5-2556 | A55 | J1 | Q154 |
| 5-2557 | A56 | J1 | Q154 |
| 5-2558 | A59 | J1 | Q154 |
| 5-2559 | A60 | J1 | Q154 |
| 5-2560 | A61 | J1 | Q154 |
| 5-2561 | A62 | J1 | Q154 |
| 5-2562 | A63 | J1 | Q154 |
| 5-2563 | A3 | J1 | Q160 |
| 5-2564 | A4 | J1 | Q160 |
| 5-2565 | A5 | J1 | Q160 |
| 5-2566 | A7 | J1 | Q160 |
| 5-2567 | A8 | J1 | Q160 |
| 5-2568 | A10 | J1 | Q160 |
| 5-2569 | A13 | J1 | Q160 |
| 5-2570 | A14 | J1 | Q160 |
| 5-2571 | A15 | J1 | Q160 |
| 5-2572 | A17 | J1 | Q160 |
| 5-2573 | A18 | J1 | Q160 |
| 5-2574 | A23 | J1 | Q160 |
| 5-2575 | A24 | J1 | Q160 |
| 5-2576 | A32 | J1 | Q160 |
| 5-2577 | A33 | J1 | Q160 |
| 5-2578 | A34 | J1 | Q160 |
| 5-2579 | A35 | J1 | Q160 |
| 5-2580 | A36 | J1 | Q160 |
| 5-2581 | A37 | J1 | Q160 |
| 5-2582 | A38 | J1 | Q160 |
| 5-2583 | A39 | J1 | Q160 |
| 5-2584 | A40 | J1 | Q160 |
| 5-2585 | A41 | J1 | Q160 |
| 5-2586 | A42 | J1 | Q160 |
| 5-2587 | A43 | J1 | Q160 |
| 5-2588 | A44 | J1 | Q160 |
| 5-2589 | A45 | J1 | Q160 |
| 5-2590 | A46 | J1 | Q160 |
| 5-2591 | A47 | J1 | Q160 |
| 5-2592 | A48 | J1 | Q160 |
| 5-2593 | A49 | J1 | Q160 |
| 5-2594 | A50 | J1 | Q160 |
| 5-2595 | A52 | J1 | Q160 |
| 5-2596 | A53 | J1 | Q160 |
| 5-2597 | A54 | J1 | Q160 |
| 5-2598 | A55 | J1 | Q160 |
| 5-2599 | A56 | J1 | Q160 |
| 5-2600 | A59 | J1 | Q160 |
| 5-2601 | A60 | J1 | Q160 |
| 5-2602 | A61 | J1 | Q160 |
| 5-2603 | A62 | J1 | Q160 |
| 5-2604 | A63 | J1 | Q160 |
| 5-2605 | A3 | J1 | Q168 |
| 5-2606 | A4 | J1 | Q168 |
| 5-2607 | A5 | J1 | Q168 |
| 5-2608 | A7 | J1 | Q168 |
| 5-2609 | A8 | J1 | Q168 |
| 5-2610 | A10 | J1 | Q168 |
| 5-2611 | A13 | J1 | Q168 |
| 5-2612 | A14 | J1 | Q168 |
| 5-2613 | A15 | J1 | Q168 |
| 5-2614 | A17 | J1 | Q168 |
| 5-2615 | A18 | J1 | Q168 |
| 5-2616 | A23 | J1 | Q168 |
| 5-2617 | A24 | J1 | Q168 |
| 5-2618 | A32 | J1 | Q168 |
| 5-2619 | A33 | J1 | Q168 |
| 5-2620 | A34 | J1 | Q168 |
| 5-2621 | A35 | J1 | Q168 |
| 5-2622 | A36 | J1 | Q168 |
| 5-2623 | A37 | J1 | Q168 |
| 5-2624 | A38 | J1 | Q168 |
| 5-2625 | A39 | J1 | Q168 |
| 5-2626 | A40 | J1 | Q168 |
| 5-2627 | A41 | J1 | Q168 |
| 5-2628 | A42 | J1 | Q168 |
| 5-2629 | A43 | J1 | Q168 |
| 5-2630 | A44 | J1 | Q168 |
| 5-2631 | A45 | J1 | Q168 |
| 5-2632 | A46 | J1 | Q168 |
| 5-2633 | A47 | J1 | Q168 |
| 5-2634 | A48 | J1 | Q168 |
| 5-2635 | A49 | J1 | Q168 |
| 5-2636 | A50 | J1 | Q168 |
| 5-2637 | A52 | J1 | Q168 |
| 5-2638 | A53 | J1 | Q168 |
| 5-2639 | A54 | J1 | Q168 |
| 5-2640 | A55 | J1 | Q168 |
| 5-2641 | A56 | J1 | Q168 |
| 5-2642 | A59 | J1 | Q168 |
| 5-2643 | A60 | J1 | Q168 |
| 5-2644 | A61 | J1 | Q168 |
| 5-2645 | A62 | J1 | Q168 |
| 5-2646 | A63 | J1 | Q168 |
| 5-2647 | A3 | J1 | Q178 |
| 5-2648 | A4 | J1 | Q178 |
| 5-2649 | A5 | J1 | Q178 |
| 5-2650 | A7 | J1 | Q178 |
| 5-2651 | A8 | J1 | Q178 |
| 5-2652 | A10 | J1 | Q178 |
| 5-2653 | A13 | J1 | Q178 |
| 5-2654 | A14 | J1 | Q178 |
| 5-2655 | A15 | J1 | Q178 |
| 5-2656 | A17 | J1 | Q178 |
| 5-2657 | A18 | J1 | Q178 |
| 5-2658 | A23 | J1 | Q178 |
| 5-2659 | A24 | J1 | Q178 |
| 5-2660 | A32 | J1 | Q178 |
| 5-2661 | A33 | J1 | Q178 |
| 5-2662 | A34 | J1 | Q178 |
| 5-2663 | A35 | J1 | Q178 |
| 5-2664 | A36 | J1 | Q178 |
| 5-2665 | A37 | J1 | Q178 |
| 5-2666 | A38 | J1 | Q178 |
| 5-2667 | A39 | J1 | Q178 |
| 5-2668 | A40 | J1 | Q178 |
| 5-2669 | A41 | J1 | Q178 |
| 5-2670 | A42 | J1 | Q178 |
| 5-2671 | A43 | J1 | Q178 |
| 5-2672 | A44 | J1 | Q178 |
| 5-2673 | A45 | J1 | Q178 |
| 5-2674 | A46 | J1 | Q178 |
| 5-2675 | A47 | J1 | Q178 |
| 5-2676 | A48 | J1 | Q178 |
| 5-2677 | A49 | J1 | Q178 |
| 5-2678 | A50 | J1 | Q178 |
| 5-2679 | A52 | J1 | Q178 |
| 5-2680 | A53 | J1 | Q178 |
| 5-2681 | A54 | J1 | Q178 |
| 5-2682 | A55 | J1 | Q178 |
| 5-2683 | A56 | J1 | Q178 |
| 5-2684 | A59 | J1 | Q178 |
| 5-2685 | A60 | J1 | Q178 |
| 5-2686 | A61 | J1 | Q178 |
| 5-2687 | A62 | J1 | Q178 |
| 5-2688 | A63 | J1 | Q178 |
| 5-2689 | A3 | J1 | Q221 |
| 5-2690 | A4 | J1 | Q221 |
| 5-2691 | A5 | J1 | Q221 |
| 5-2692 | A7 | J1 | Q221 |
| 5-2693 | A8 | J1 | Q221 |
| 5-2694 | A10 | J1 | Q221 |
| 5-2695 | A13 | J1 | Q221 |
| 5-2696 | A14 | J1 | Q221 |
| 5-2697 | A15 | J1 | Q221 |
| 5-2698 | A17 | J1 | Q221 |
| 5-2699 | A18 | J1 | Q221 |
| 5-2700 | A23 | J1 | Q221 |
| 5-2701 | A24 | J1 | Q221 |
| 5-2702 | A32 | J1 | Q221 |
| 5-2703 | A33 | J1 | Q221 |
| 5-2704 | A34 | J1 | Q221 |
| 5-2705 | A35 | J1 | Q221 |
| 5-2706 | A36 | J1 | Q221 |
| 5-2707 | A37 | J1 | Q221 |
| 5-2708 | A38 | J1 | Q221 |
| 5-2709 | A39 | J1 | Q221 |
| 5-2710 | A40 | J1 | Q221 |
| 5-2711 | A41 | J1 | Q221 |
| 5-2712 | A42 | J1 | Q221 |
| 5-2713 | A43 | J1 | Q221 |
| 5-2714 | A44 | J1 | Q221 |
| 5-2715 | A45 | J1 | Q221 |
| 5-2716 | A46 | J1 | Q221 |
| 5-2717 | A47 | J1 | Q221 |
| 5-2718 | A48 | J1 | Q221 |
| 5-2719 | A49 | J1 | Q221 |
| 5-2720 | A50 | J1 | Q221 |
| 5-2721 | A52 | J1 | Q221 |
| 5-2722 | A53 | J1 | Q221 |
| 5-2723 | A54 | J1 | Q221 |
| 5-2724 | A55 | J1 | Q221 |
| 5-2725 | A56 | J1 | Q221 |
| 5-2726 | A59 | J1 | Q221 |
| 5-2727 | A60 | J1 | Q221 |
| 5-2728 | A61 | J1 | Q221 |
| 5-2729 | A62 | J1 | Q221 |
| 5-2730 | A63 | J1 | Q221 |
| 5-2731 | A9 | J2 | Q1 |
| 5-2732 | A9 | J2 | Q2 |
| 5-2733 | A9 | J2 | Q3 |
| 5-2734 | A9 | J2 | Q4 |
| 5-2735 | A9 | J2 | Q5 |
| 5-2736 | A9 | J2 | Q6 |
| 5-2737 | A9 | J2 | Q7 |
| 5-2738 | A9 | J2 | Q8 |
| 5-2739 | A9 | J2 | Q9 |
| 5-2740 | A9 | J2 | Q10 |
| 5-2741 | A9 | J2 | Q11 |
| 5-2742 | A9 | J2 | Q12 |
| 5-2743 | A9 | J2 | Q13 |
| 5-2744 | A9 | J2 | Q14 |
| 5-2745 | A9 | J2 | Q15 |
| 5-2746 | A9 | J2 | Q16 |
| 5-2747 | A9 | J2 | Q17 |
| 5-2748 | A9 | J2 | Q18 |
| 5-2749 | A9 | J2 | Q19 |
| 5-2750 | A9 | J2 | Q20 |
| 5-2751 | A9 | J2 | Q21 |
| 5-2752 | A9 | J2 | Q23 |
| 5-2753 | A9 | J2 | Q24 |
| 5-2754 | A9 | J2 | Q25 |
| 5-2755 | A9 | J2 | Q26 |
| 5-2756 | A9 | J2 | Q27 |
| 5-2757 | A9 | J2 | Q28 |
| 5-2758 | A9 | J2 | Q29 |
| 5-2759 | A9 | J2 | Q30 |
| 5-2760 | A9 | J2 | Q31 |
| 5-2761 | A9 | J2 | Q32 |
| 5-2762 | A9 | J2 | Q33 |
| 5-2763 | A9 | J2 | Q34 |
| 5-2764 | A9 | J2 | Q35 |
| 5-2765 | A9 | J2 | Q36 |
| 5-2766 | A9 | J2 | Q37 |
| 5-2767 | A9 | J2 | Q38 |
| 5-2768 | A9 | J2 | Q39 |
| 5-2769 | A9 | J2 | Q40 |
| 5-2770 | A9 | J2 | Q41 |
| 5-2771 | A9 | J2 | Q42 |
| 5-2772 | A9 | J2 | Q43 |
| 5-2773 | A9 | J2 | Q44 |
| 5-2774 | A9 | J2 | Q45 |
| 5-2775 | A9 | J2 | Q46 |
| 5-2776 | A9 | J2 | Q47 |
| 5-2777 | A9 | J2 | Q48 |
| 5-2778 | A9 | J2 | Q49 |
| 5-2779 | A9 | J2 | Q50 |
| 5-2780 | A9 | J2 | Q51 |
| 5-2781 | A9 | J2 | Q53 |
| 5-2782 | A9 | J2 | Q54 |
| 5-2783 | A9 | J2 | Q55 |
| 5-2784 | A9 | J2 | Q57 |
| 5-2785 | A9 | J2 | Q60 |
| 5-2786 | A9 | J2 | Q61 |
| 5-2787 | A9 | J2 | Q62 |
| 5-2788 | A9 | J2 | Q63 |
| 5-2789 | A9 | J2 | Q64 |
| 5-2790 | A9 | J2 | Q67 |
| 5-2791 | A9 | J2 | Q68 |
| 5-2792 | A9 | J2 | Q69 |
| 5-2793 | A9 | J2 | Q71 |
| 5-2794 | A9 | J2 | Q72 |
| 5-2795 | A9 | J2 | Q73 |
| 5-2796 | A9 | J2 | Q74 |
| 5-2797 | A9 | J2 | Q75 |
| 5-2798 | A9 | J2 | Q76 |
| 5-2799 | A9 | J2 | Q77 |
| 5-2800 | A9 | J2 | Q82 |
| 5-2801 | A9 | J2 | Q83 |
| 5-2802 | A9 | J2 | Q84 |
| 5-2803 | A9 | J2 | Q85 |
| 5-2804 | A9 | J2 | Q86 |
| 5-2805 | A9 | J2 | Q87 |
| 5-2806 | A9 | J2 | Q88 |
| 5-2807 | A9 | J2 | Q89 |
| 5-2808 | A9 | J2 | Q90 |
| 5-2809 | A9 | J2 | Q91 |
| 5-2810 | A9 | J2 | Q92 |
| 5-2811 | A9 | J2 | Q93 |
| 5-2812 | A9 | J2 | Q94 |
| 5-2813 | A9 | J2 | Q95 |
| 5-2814 | A9 | J2 | Q96 |
| 5-2815 | A9 | J2 | Q97 |
| 5-2816 | A9 | J2 | Q99 |
| 5-2817 | A9 | J2 | Q100 |
| 5-2818 | A9 | J2 | Q101 |
| 5-2819 | A9 | J2 | Q102 |
| 5-2820 | A9 | J2 | Q103 |
| 5-2821 | A9 | J2 | Q104 |
| 5-2822 | A9 | J2 | Q105 |
| 5-2823 | A9 | J2 | Q107 |
| 5-2824 | A9 | J2 | Q108 |
| 5-2825 | A9 | J2 | Q109 |
| 5-2826 | A9 | J2 | Q110 |
| 5-2827 | A9 | J2 | Q111 |
| 5-2828 | A9 | J2 | Q112 |
| 5-2829 | A9 | J2 | Q113 |
| 5-2830 | A9 | J2 | Q114 |
| 5-2831 | A9 | J2 | Q115 |
| 5-2832 | A9 | J2 | Q116 |
| 5-2833 | A9 | J2 | Q117 |
| 5-2834 | A9 | J2 | Q118 |
| 5-2835 | A9 | J2 | Q119 |
| 5-2836 | A9 | J2 | Q120 |
| 5-2837 | A9 | J2 | Q121 |
| 5-2838 | A9 | J2 | Q122 |
| 5-2839 | A9 | J2 | Q126 |
| 5-2840 | A9 | J2 | Q127 |
| 5-2841 | A9 | J2 | Q128 |
| 5-2842 | A9 | J2 | Q129 |
| 5-2843 | A9 | J2 | Q130 |
| 5-2844 | A9 | J2 | Q131 |
| 5-2845 | A9 | J2 | Q132 |
| 5-2846 | A9 | J2 | Q133 |
| 5-2847 | A9 | J2 | Q134 |
| 5-2848 | A9 | J2 | Q135 |
| 5-2849 | A9 | J2 | Q136 |
| 5-2850 | A9 | J2 | Q137 |
| 5-2851 | A9 | J2 | Q138 |
| 5-2852 | A9 | J2 | Q139 |
| 5-2853 | A9 | J2 | Q140 |
| 5-2854 | A9 | J2 | Q143 |
| 5-2855 | A9 | J2 | Q144 |
| 5-2856 | A9 | J2 | Q145 |
| 5-2857 | A9 | J2 | Q146 |
| 5-2858 | A9 | J2 | Q148 |
| 5-2859 | A9 | J2 | Q149 |
| 5-2860 | A9 | J2 | Q153 |
| 5-2861 | A9 | J2 | Q155 |
| 5-2862 | A9 | J2 | Q156 |
| 5-2863 | A9 | J2 | Q157 |
| 5-2864 | A9 | J2 | Q158 |
| 5-2865 | A9 | J2 | Q159 |
| 5-2866 | A9 | J2 | Q161 |
| 5-2867 | A9 | J2 | Q162 |
| 5-2868 | A9 | J2 | Q163 |
| 5-2869 | A9 | J2 | Q164 |
| 5-2870 | A9 | J2 | Q165 |
| 5-2871 | A9 | J2 | Q166 |
| 5-2872 | A9 | J2 | Q167 |
| 5-2873 | A9 | J2 | Q168 |
| 5-2874 | A9 | J2 | Q169 |
| 5-2875 | A9 | J2 | Q170 |
| 5-2876 | A9 | J2 | Q171 |
| 5-2877 | A9 | J2 | Q172 |
| 5-2878 | A9 | J2 | Q173 |
| 5-2879 | A9 | J2 | Q174 |
| 5-2880 | A9 | J2 | Q175 |
| 5-2881 | A9 | J2 | Q176 |
| 5-2882 | A9 | J2 | Q177 |
| 5-2883 | A9 | J2 | Q178 |
| 5-2884 | A9 | J2 | Q179 |
| 5-2885 | A9 | J2 | Q181 |
| 5-2886 | A9 | J2 | Q182 |
| 5-2887 | A9 | J2 | Q183 |
| 5-2888 | A9 | J2 | Q184 |
| 5-2889 | A9 | J2 | Q185 |
| 5-2890 | A9 | J2 | Q186 |
| 5-2891 | A9 | J2 | Q187 |
| 5-2892 | A9 | J2 | Q188 |
| 5-2893 | A9 | J2 | Q189 |
| 5-2894 | A9 | J2 | Q190 |
| 5-2895 | A9 | J2 | Q191 |
| 5-2896 | A9 | J2 | Q192 |
| 5-2897 | A9 | J2 | Q193 |
| 5-2898 | A9 | J2 | Q194 |
| 5-2899 | A9 | J2 | Q195 |
| 5-2900 | A9 | J2 | Q196 |
| 5-2901 | A9 | J2 | Q197 |
| 5-2902 | A9 | J2 | Q198 |
| 5-2903 | A9 | J2 | Q199 |
| 5-2904 | A9 | J2 | Q200 |
| 5-2905 | A9 | J2 | Q201 |
| 5-2906 | A9 | J2 | Q202 |
| 5-2907 | A9 | J2 | Q203 |
| 5-2908 | A9 | J2 | Q204 |
| 5-2909 | A9 | J2 | Q205 |
| 5-2910 | A9 | J2 | Q206 |
| 5-2911 | A9 | J2 | Q207 |
| 5-2912 | A9 | J2 | Q208 |
| 5-2913 | A9 | J2 | Q209 |
| 5-2914 | A9 | J2 | Q210 |
| 5-2915 | A9 | J2 | Q211 |
| 5-2916 | A9 | J2 | Q212 |
| 5-2917 | A9 | J2 | Q213 |
| 5-2918 | A9 | J2 | Q214 |
| 5-2919 | A9 | J2 | Q215 |
| 5-2920 | A9 | J2 | Q216 |
| 5-2921 | A9 | J2 | Q217 |
| 5-2922 | A9 | J2 | Q222 |
| 5-2923 | A9 | J2 | Q223 |
| 5-2924 | A9 | J2 | Q224 |
| 5-2925 | A9 | J2 | Q225 |
| 5-2926 | A9 | J2 | Q226 |
| 5-2927 | A9 | J2 | Q227 |
| 5-2928 | A9 | J2 | Q228 |
| 5-2929 | A9 | J2 | Q229 |
| 5-2930 | A9 | J2 | Q230 |
| 5-2931 | A9 | J2 | Q231 |
| 5-2932 | A9 | J2 | Q232 |
| 5-2933 | A9 | J2 | Q233 |
| 5-2934 | A9 | J2 | Q234 |
| 5-2935 | A9 | J2 | Q235 |
| 5-2936 | A9 | J2 | Q236 |
| 5-2937 | A9 | J2 | Q237 |
| 5-2938 | A9 | J2 | Q238 |
| 5-2939 | A9 | J2 | Q239 |
| 5-2940 | A9 | J2 | Q240 |
| 5-2941 | A9 | J2 | Q241 |
| 5-2942 | A9 | J2 | Q242 |
| 5-2943 | A9 | J2 | Q243 |
| 5-2944 | A9 | J2 | Q244 |
| 5-2945 | A9 | J2 | Q246 |
| 5-2946 | A9 | J2 | Q247 |
| 5-2947 | A9 | J2 | Q249 |
| 5-2948 | A9 | J2 | Q250 |
| 5-2949 | A9 | J2 | Q251 |
| 5-2950 | A9 | J2 | Q256 |
| 5-2951 | A9 | J2 | Q258 |
| 5-2952 | A9 | J2 | Q259 |
| 5-2953 | A9 | J2 | Q260 |
| 5-2954 | A9 | J2 | Q261 |
| 5-2955 | A9 | J2 | Q262 |
| 5-2956 | A9 | J2 | Q263 |
| 5-2957 | A9 | J2 | Q264 |
| 5-2958 | A9 | J2 | Q266 |
| 5-2959 | A9 | J2 | Q267 |
| 5-2960 | A9 | J2 | Q268 |
| 5-2961 | A9 | J2 | Q269 |
| 5-2962 | A9 | J2 | Q270 |
| 5-2963 | A9 | J2 | Q271 |
| 5-2964 | A9 | J2 | Q272 |
| 5-2965 | A9 | J2 | Q274 |
| 5-2966 | A9 | J2 | Q275 |
| 5-2967 | A21 | J2 | Q1 |
| 5-2968 | A21 | J2 | Q2 |
| 5-2969 | A21 | J2 | Q3 |
| 5-2970 | A21 | J2 | Q4 |
| 5-2971 | A21 | J2 | Q5 |
| 5-2972 | A21 | J2 | Q6 |
| 5-2973 | A21 | J2 | Q7 |
| 5-2974 | A21 | J2 | Q8 |
| 5-2975 | A21 | J2 | Q9 |
| 5-2976 | A21 | J2 | Q10 |
| 5-2977 | A21 | J2 | Q11 |
| 5-2978 | A21 | J2 | Q12 |
| 5-2979 | A21 | J2 | Q13 |
| 5-2980 | A21 | J2 | Q14 |
| 5-2981 | A21 | J2 | Q15 |
| 5-2982 | A21 | J2 | Q16 |
| 5-2983 | A21 | J2 | Q17 |
| 5-2984 | A21 | J2 | Q18 |
| 5-2985 | A21 | J2 | Q19 |
| 5-2986 | A21 | J2 | Q20 |
| 5-2987 | A21 | J2 | Q21 |
| 5-2988 | A21 | J2 | Q23 |
| 5-2989 | A21 | J2 | Q24 |
| 5-2990 | A21 | J2 | Q25 |
| 5-2991 | A21 | J2 | Q26 |
| 5-2992 | A21 | J2 | Q27 |
| 5-2993 | A21 | J2 | Q28 |
| 5-2994 | A21 | J2 | Q29 |
| 5-2995 | A21 | J2 | Q30 |
| 5-2996 | A21 | J2 | Q31 |
| 5-2997 | A21 | J2 | Q32 |
| 5-2998 | A21 | J2 | Q33 |
| 5-2999 | A21 | J2 | Q34 |
| 5-3000 | A21 | J2 | Q35 |
| 5-3001 | A21 | J2 | Q36 |
| 5-3002 | A21 | J2 | Q37 |
| 5-3003 | A21 | J2 | Q38 |
| 5-3004 | A21 | J2 | Q39 |
| 5-3005 | A21 | J2 | Q40 |
| 5-3006 | A21 | J2 | Q41 |
| 5-3007 | A21 | J2 | Q42 |
| 5-3008 | A21 | J2 | Q43 |
| 5-3009 | A21 | J2 | Q44 |
| 5-3010 | A21 | J2 | Q45 |
| 5-3011 | A21 | J2 | Q46 |
| 5-3012 | A21 | J2 | Q47 |
| 5-3013 | A21 | J2 | Q48 |
| 5-3014 | A21 | J2 | Q49 |
| 5-3015 | A21 | J2 | Q50 |
| 5-3016 | A21 | J2 | Q51 |
| 5-3017 | A21 | J2 | Q53 |
| 5-3018 | A21 | J2 | Q54 |
| 5-3019 | A21 | J2 | Q55 |
| 5-3020 | A21 | J2 | Q57 |
| 5-3021 | A21 | J2 | Q60 |
| 5-3022 | A21 | J2 | Q61 |
| 5-3023 | A21 | J2 | Q62 |
| 5-3024 | A21 | J2 | Q63 |
| 5-3025 | A21 | J2 | Q64 |
| 5-3026 | A21 | J2 | Q67 |
| 5-3027 | A21 | J2 | Q68 |
| 5-3028 | A21 | J2 | Q69 |
| 5-3029 | A21 | J2 | Q71 |
| 5-3030 | A21 | J2 | Q72 |
| 5-3031 | A21 | J2 | Q73 |
| 5-3032 | A21 | J2 | Q74 |
| 5-3033 | A21 | J2 | Q75 |
| 5-3034 | A21 | J2 | Q76 |
| 5-3035 | A21 | J2 | Q77 |
| 5-3036 | A21 | J2 | Q82 |
| 5-3037 | A21 | J2 | Q83 |
| 5-3038 | A21 | J2 | Q84 |
| 5-3039 | A21 | J2 | Q85 |
| 5-3040 | A21 | J2 | Q86 |
| 5-3041 | A21 | J2 | Q87 |
| 5-3042 | A21 | J2 | Q88 |
| 5-3043 | A21 | J2 | Q89 |
| 5-3044 | A21 | J2 | Q90 |
| 5-3045 | A21 | J2 | Q91 |
| 5-3046 | A21 | J2 | Q92 |
| 5-3047 | A21 | J2 | Q93 |
| 5-3048 | A21 | J2 | Q94 |
| 5-3049 | A21 | J2 | Q95 |
| 5-3050 | A21 | J2 | Q96 |
| 5-3051 | A21 | J2 | Q97 |
| 5-3052 | A21 | J2 | Q99 |
| 5-3053 | A21 | J2 | Q100 |
| 5-3054 | A21 | J2 | Q101 |
| 5-3055 | A21 | J2 | Q102 |
| 5-3056 | A21 | J2 | Q103 |
| 5-3057 | A21 | J2 | Q104 |
| 5-3058 | A21 | J2 | Q105 |
| 5-3059 | A21 | J2 | Q107 |
| 5-3060 | A21 | J2 | Q108 |
| 5-3061 | A21 | J2 | Q109 |
| 5-3062 | A21 | J2 | Q110 |
| 5-3063 | A21 | J2 | Q111 |
| 5-3064 | A21 | J2 | Q112 |
| 5-3065 | A21 | J2 | Q113 |
| 5-3066 | A21 | J2 | Q114 |
| 5-3067 | A21 | J2 | Q115 |
| 5-3068 | A21 | J2 | Q116 |
| 5-3069 | A21 | J2 | Q117 |
| 5-3070 | A21 | J2 | Q118 |
| 5-3071 | A21 | J2 | Q119 |
| 5-3072 | A21 | J2 | Q120 |
| 5-3073 | A21 | J2 | Q121 |
| 5-3074 | A21 | J2 | Q122 |
| 5-3075 | A21 | J2 | Q126 |
| 5-3076 | A21 | J2 | Q127 |
| 5-3077 | A21 | J2 | Q128 |
| 5-3078 | A21 | J2 | Q129 |
| 5-3079 | A21 | J2 | Q130 |
| 5-3080 | A21 | J2 | Q131 |
| 5-3081 | A21 | J2 | Q132 |
| 5-3082 | A21 | J2 | Q133 |
| 5-3083 | A21 | J2 | Q134 |
| 5-3084 | A21 | J2 | Q135 |
| 5-3085 | A21 | J2 | Q136 |
| 5-3086 | A21 | J2 | Q137 |
| 5-3087 | A21 | J2 | Q138 |
| 5-3088 | A21 | J2 | Q139 |
| 5-3089 | A21 | J2 | Q140 |
| 5-3090 | A21 | J2 | Q143 |
| 5-3091 | A21 | J2 | Q144 |
| 5-3092 | A21 | J2 | Q145 |
| 5-3093 | A21 | J2 | Q146 |
| 5-3094 | A21 | J2 | Q148 |
| 5-3095 | A21 | J2 | Q149 |
| 5-3096 | A21 | J2 | Q153 |
| 5-3097 | A21 | J2 | Q155 |
| 5-3098 | A21 | J2 | Q156 |
| 5-3099 | A21 | J2 | Q157 |
| 5-3100 | A21 | J2 | Q158 |
| 5-3101 | A21 | J2 | Q159 |
| 5-3102 | A21 | J2 | Q161 |
| 5-3103 | A21 | J2 | Q162 |
| 5-3104 | A21 | J2 | Q163 |
| 5-3105 | A21 | J2 | Q164 |
| 5-3106 | A21 | J2 | Q165 |
| 5-3107 | A21 | J2 | Q166 |
| 5-3108 | A21 | J2 | Q167 |
| 5-3109 | A21 | J2 | Q168 |
| 5-3110 | A21 | J2 | Q169 |
| 5-3111 | A21 | J2 | Q170 |
| 5-3112 | A21 | J2 | Q171 |
| 5-3113 | A21 | J2 | Q172 |
| 5-3114 | A21 | J2 | Q173 |
| 5-3115 | A21 | J2 | Q174 |
| 5-3116 | A21 | J2 | Q175 |
| 5-3117 | A21 | J2 | Q176 |
| 5-3118 | A21 | J2 | Q177 |
| 5-3119 | A21 | J2 | Q178 |
| 5-3120 | A21 | J2 | Q179 |
| 5-3121 | A21 | J2 | Q181 |
| 5-3122 | A21 | J2 | Q182 |
| 5-3123 | A21 | J2 | Q183 |
| 5-3124 | A21 | J2 | Q184 |
| 5-3125 | A21 | J2 | Q185 |
| 5-3126 | A21 | J2 | Q186 |
| 5-3127 | A21 | J2 | Q187 |
| 5-3128 | A21 | J2 | Q188 |
| 5-3129 | A21 | J2 | Q189 |
| 5-3130 | A21 | J2 | Q190 |
| 5-3131 | A21 | J2 | Q191 |
| 5-3132 | A21 | J2 | Q192 |
| 5-3133 | A21 | J2 | Q193 |
| 5-3134 | A21 | J2 | Q194 |
| 5-3135 | A21 | J2 | Q195 |
| 5-3136 | A21 | J2 | Q196 |
| 5-3137 | A21 | J2 | Q197 |
| 5-3138 | A21 | J2 | Q198 |
| 5-3139 | A21 | J2 | Q199 |
| 5-3140 | A21 | J2 | Q200 |
| 5-3141 | A21 | J2 | Q201 |
| 5-3142 | A21 | J2 | Q202 |
| 5-3143 | A21 | J2 | Q203 |
| 5-3144 | A21 | J2 | Q204 |
| 5-3145 | A21 | J2 | Q205 |
| 5-3146 | A21 | J2 | Q206 |
| 5-3147 | A21 | J2 | Q207 |
| 5-3148 | A21 | J2 | Q208 |
| 5-3149 | A21 | J2 | Q209 |
| 5-3150 | A21 | J2 | Q210 |
| 5-3151 | A21 | J2 | Q211 |
| 5-3152 | A21 | J2 | Q212 |
| 5-3153 | A21 | J2 | Q213 |
| 5-3154 | A21 | J2 | Q214 |
| 5-3155 | A21 | J2 | Q215 |
| 5-3156 | A21 | J2 | Q216 |
| 5-3157 | A21 | J2 | Q217 |
| 5-3158 | A21 | J2 | Q222 |
| 5-3159 | A21 | J2 | Q223 |
| 5-3160 | A21 | J2 | Q224 |
| 5-3161 | A21 | J2 | Q225 |
| 5-3162 | A21 | J2 | Q226 |
| 5-3163 | A21 | J2 | Q227 |
| 5-3164 | A21 | J2 | Q228 |
| 5-2165 | A21 | J2 | Q229 |
| 5-3166 | A21 | J2 | Q230 |
| 5-3167 | A21 | J2 | Q231 |
| 5-3168 | A21 | J2 | Q232 |
| 5-3169 | A21 | J2 | Q233 |
| 5-3170 | A21 | J2 | Q234 |
| 5-3171 | A21 | J2 | Q235 |
| 5-3172 | A21 | J2 | Q236 |
| 5-3173 | A21 | J2 | Q237 |
| 5-3174 | A21 | J2 | Q238 |
| 5-3175 | A21 | J2 | Q239 |
| 5-3176 | A21 | J2 | Q240 |
| 5-3177 | A21 | J2 | Q241 |
| 5-3178 | A21 | J2 | Q242 |
| 5-3179 | A21 | J2 | Q243 |
| 5-3180 | A21 | J2 | Q244 |
| 5-3181 | A21 | J2 | Q246 |
| 5-3182 | A21 | J2 | Q247 |
| 5-3183 | A21 | J2 | Q249 |
| 5-3184 | A21 | J2 | Q250 |
| 5-3185 | A21 | J2 | Q251 |
| 5-3186 | A21 | J2 | Q256 |
| 5-3187 | A21 | J2 | Q258 |
| 5-3188 | A21 | J2 | Q259 |
| 5-3189 | A21 | J2 | Q260 |
| 5-3190 | A21 | J2 | Q261 |
| 5-3191 | A21 | J2 | Q262 |
| 5-3192 | A21 | J2 | Q263 |
| 5-3193 | A21 | J2 | Q264 |
| 5-3194 | A21 | J2 | Q266 |
| 5-3195 | A21 | J2 | Q267 |
| 5-3196 | A21 | J2 | Q268 |
| 5-3197 | A21 | J2 | Q269 |
| 5-3198 | A21 | J2 | Q270 |
| 5-3199 | A21 | J2 | Q271 |
| 5-3200 | A21 | J2 | Q272 |
| 5-3201 | A21 | J2 | Q274 |
| 5-3202 | A21 | J2 | Q275 |
| 5-3203 | A22 | J2 | Q1 |
| 5-3204 | A22 | J2 | Q2 |
| 5-3205 | A22 | J2 | Q3 |
| 5-3206 | A22 | J2 | Q4 |
| 5-3207 | A22 | J2 | Q5 |
| 5-3208 | A22 | J2 | Q6 |
| 5-3209 | A22 | J2 | Q7 |
| 5-3210 | A22 | J2 | Q8 |
| 5-3211 | A22 | J2 | Q9 |
| 5-3212 | A22 | J2 | Q10 |
| 5-3213 | A22 | J2 | Q11 |
| 5-3214 | A22 | J2 | Q12 |
| 5-3215 | A22 | J2 | Q13 |
| 5-3216 | A22 | J2 | Q14 |
| 5-3217 | A22 | J2 | Q15 |
| 5-3218 | A22 | J2 | Q16 |
| 5-3219 | A22 | J2 | Q17 |
| 5-3220 | A22 | J2 | Q18 |
| 5-3221 | A22 | J2 | Q19 |
| 5-3222 | A22 | J2 | Q20 |
| 5-3223 | A22 | J2 | Q21 |
| 5-3224 | A22 | J2 | Q23 |
| 5-3225 | A22 | J2 | Q24 |
| 5-3226 | A22 | J2 | Q25 |
| 5-3227 | A22 | J2 | Q26 |
| 5-3228 | A22 | J2 | Q27 |
| 5-3229 | A22 | J2 | Q28 |
| 5-3230 | A22 | J2 | Q29 |
| 5-3231 | A22 | J2 | Q30 |
| 5-3232 | A22 | J2 | Q31 |
| 5-3233 | A22 | J2 | Q32 |
| 5-3234 | A22 | J2 | Q33 |
| 5-3235 | A22 | J2 | Q34 |
| 5-3236 | A22 | J2 | Q35 |
| 5-3237 | A22 | J2 | Q36 |
| 5-3238 | A22 | J2 | Q37 |
| 5-2239 | A22 | J2 | Q38 |
| 5-3240 | A22 | J2 | Q39 |
| 5-3241 | A22 | J2 | Q40 |
| 5-3242 | A22 | J2 | Q41 |
| 5-3243 | A22 | J2 | Q42 |
| 5-3244 | A22 | J2 | Q43 |
| 5-3245 | A22 | J2 | Q44 |
| 5-3246 | A22 | J2 | Q45 |
| 5-3247 | A22 | J2 | Q46 |
| 5-3248 | A22 | J2 | Q47 |
| 5-3249 | A22 | J2 | Q48 |
| 5-3250 | A22 | J2 | Q49 |
| 5-3251 | A22 | J2 | Q50 |
| 5-3252 | A22 | J2 | Q51 |
| 5-3253 | A22 | J2 | Q53 |
| 5-3254 | A22 | J2 | Q54 |
| 5-3255 | A22 | J2 | Q55 |
| 5-3256 | A22 | J2 | Q57 |
| 5-3257 | A22 | J2 | Q60 |
| 5-3258 | A22 | J2 | Q61 |
| 5-3259 | A22 | J2 | Q62 |
| 5-3260 | A22 | J2 | Q63 |
| 5-3261 | A22 | J2 | Q64 |
| 5-3262 | A22 | J2 | Q67 |
| 5-3263 | A22 | J2 | Q68 |
| 5-3264 | A22 | J2 | Q69 |
| 5-3265 | A22 | J2 | Q71 |
| 5-3266 | A22 | J2 | Q72 |
| 5-3267 | A22 | J2 | Q73 |
| 5-3268 | A22 | J2 | Q74 |
| 5-3269 | A22 | J2 | Q75 |
| 5-3270 | A22 | J2 | Q76 |
| 5-3271 | A22 | J2 | Q77 |
| 5-3272 | A22 | J2 | Q82 |
| 5-3273 | A22 | J2 | Q83 |
| 5-3274 | A22 | J2 | Q84 |
| 5-3275 | A22 | J2 | Q85 |
| 5-3276 | A22 | J2 | Q86 |
| 5-3277 | A22 | J2 | Q87 |
| 5-3278 | A22 | J2 | Q88 |
| 5-3279 | A22 | J2 | Q89 |
| 5-3280 | A22 | J2 | Q90 |
| 5-3281 | A22 | J2 | Q91 |
| 5-3282 | A22 | J2 | Q92 |
| 5-3283 | A22 | J2 | Q93 |
| 5-3284 | A22 | J2 | Q94 |
| 5-3285 | A22 | J2 | Q95 |
| 5-3286 | A22 | J2 | Q96 |
| 5-3287 | A22 | J2 | Q97 |
| 5-3288 | A22 | J2 | Q99 |
| 5-3289 | A22 | J2 | Q100 |
| 5-3290 | A22 | J2 | Q101 |
| 5-3291 | A22 | J2 | Q102 |
| 5-3292 | A22 | J2 | Q103 |
| 5-3293 | A22 | J2 | Q104 |
| 5-3294 | A22 | J2 | Q105 |
| 5-3295 | A22 | J2 | Q107 |
| 5-3296 | A22 | J2 | Q108 |
| 5-3297 | A22 | J2 | Q109 |
| 5-3298 | A22 | J2 | Q110 |
| 5-3299 | A22 | J2 | Q111 |
| 5-3300 | A22 | J2 | Q112 |
| 5-3301 | A22 | J2 | Q113 |
| 5-3302 | A22 | J2 | Q114 |
| 5-3303 | A22 | J2 | Q115 |
| 5-3304 | A22 | J2 | Q116 |
| 5-3305 | A22 | J2 | Q117 |
| 5-3306 | A22 | J2 | Q118 |
| 5-3307 | A22 | J2 | Q119 |
| 5-3308 | A22 | J2 | Q120 |
| 5-3309 | A22 | J2 | Q121 |
| 5-3310 | A22 | J2 | Q122 |
| 5-3311 | A22 | J2 | Q126 |
| 5-3312 | A22 | J2 | Q127 |
| 5-3313 | A22 | J2 | Q128 |
| 5-3314 | A22 | J2 | Q129 |
| 5-3315 | A22 | J2 | Q130 |
| 5-3316 | A22 | J2 | Q131 |
| 5-3317 | A22 | J2 | Q132 |
| 5-3318 | A22 | J2 | Q133 |
| 5-3319 | A22 | J2 | Q134 |
| 5-3320 | A22 | J2 | Q135 |
| 5-3321 | A22 | J2 | Q136 |
| 5-3322 | A22 | J2 | Q137 |
| 5-3323 | A22 | J2 | Q138 |
| 5-3324 | A22 | J2 | Q139 |
| 5-3325 | A22 | J2 | Q140 |
| 5-3326 | A22 | J2 | Q143 |
| 5-3327 | A22 | J2 | Q144 |
| 5-3328 | A22 | J2 | Q145 |
| 5-3329 | A22 | J2 | Q146 |
| 5-3330 | A22 | J2 | Q148 |
| 5-3331 | A22 | J2 | Q149 |
| 5-3332 | A22 | J2 | Q153 |
| 5-3333 | A22 | J2 | Q155 |
| 5-3334 | A22 | J2 | Q156 |
| 5-3335 | A22 | J2 | Q157 |
| 5-3336 | A22 | J2 | Q158 |
| 5-3337 | A22 | J2 | Q159 |
| 5-3338 | A22 | J2 | Q161 |
| 5-3339 | A22 | J2 | Q162 |
| 5-3340 | A22 | J2 | Q163 |
| 5-3341 | A22 | J2 | Q164 |
| 5-3342 | A22 | J2 | Q165 |
| 5-3343 | A22 | J2 | Q166 |
| 5-3344 | A22 | J2 | Q167 |
| 5-3345 | A22 | J2 | Q168 |
| 5-3346 | A22 | J2 | Q169 |
| 5-3347 | A22 | J2 | Q170 |
| 5-3348 | A22 | J2 | Q171 |
| 5-3349 | A22 | J2 | Q172 |
| 5-3350 | A22 | J2 | Q173 |
| 5-3351 | A22 | J2 | Q174 |
| 5-3352 | A22 | J2 | Q175 |
| 5-3353 | A22 | J2 | Q176 |
| 5-3354 | A22 | J2 | Q177 |
| 5-3355 | A22 | J2 | Q178 |
| 5-3356 | A22 | J2 | Q179 |
| 5-3357 | A22 | J2 | Q181 |
| 5-3358 | A22 | J2 | Q182 |
| 5-3359 | A22 | J2 | Q183 |
| 5-3360 | A22 | J2 | Q184 |
| 5-3361 | A22 | J2 | Q185 |
| 5-3362 | A22 | J2 | Q186 |
| 5-3363 | A22 | J2 | Q187 |
| 5-3364 | A22 | J2 | Q188 |
| 5-3365 | A22 | J2 | Q189 |
| 5-3366 | A22 | J2 | Q190 |
| 5-3367 | A22 | J2 | Q191 |
| 5-3368 | A22 | J2 | Q192 |
| 5-3369 | A22 | J2 | Q193 |
| 5-3370 | A22 | J2 | Q194 |
| 5-3371 | A22 | J2 | Q195 |
| 5-3372 | A22 | J2 | Q196 |
| 5-3373 | A22 | J2 | Q197 |
| 5-3374 | A22 | J2 | Q198 |
| 5-3375 | A22 | J2 | Q199 |
| 5-3376 | A22 | J2 | Q200 |
| 5-3377 | A22 | J2 | Q201 |
| 5-3378 | A22 | J2 | Q202 |
| 5-3379 | A22 | J2 | Q203 |
| 5-3380 | A22 | J2 | Q204 |
| 5-3381 | A22 | J2 | Q205 |
| 5-3382 | A22 | J2 | Q206 |
| 5-3383 | A22 | J2 | Q207 |
| 5-3384 | A22 | J2 | Q208 |
| 5-3385 | A22 | J2 | Q209 |
| 5-3386 | A22 | J2 | Q210 |
| 5-3387 | A22 | J2 | Q211 |
| 5-3388 | A22 | J2 | Q212 |
| 5-3389 | A22 | J2 | Q213 |
| 5-3390 | A22 | J2 | Q214 |
| 5-3391 | A22 | J2 | Q215 |
| 5-3392 | A22 | J2 | Q216 |
| 5-3393 | A22 | J2 | Q217 |
| 5-3394 | A22 | J2 | Q222 |
| 5-3395 | A22 | J2 | Q223 |
| 5-3396 | A22 | J2 | Q224 |
| 5-3397 | A22 | J2 | Q225 |
| 5-3398 | A22 | J2 | Q226 |
| 5-3399 | A22 | J2 | Q227 |
| 5-3400 | A22 | J2 | Q228 |
| 5-3401 | A22 | J2 | Q229 |
| 5-3402 | A22 | J2 | Q230 |
| 5-3403 | A22 | J2 | Q231 |
| 5-3404 | A22 | J2 | Q232 |
| 5-3405 | A22 | J2 | Q233 |
| 5-3406 | A22 | J2 | Q234 |
| 5-3407 | A22 | J2 | Q235 |
| 5-3408 | A22 | J2 | Q236 |
| 5-3409 | A22 | J2 | Q237 |
| 5-3410 | A22 | J2 | Q238 |
| 5-3411 | A22 | J2 | Q239 |
| 5-3412 | A22 | J2 | Q240 |
| 5-3413 | A22 | J2 | Q241 |
| 5-3414 | A22 | J2 | Q242 |
| 5-3415 | A22 | J2 | Q243 |
| 5-3416 | A22 | J2 | Q244 |
| 5-3417 | A22 | J2 | Q246 |
| 5-3418 | A22 | J2 | Q247 |
| 5-3419 | A22 | J2 | Q249 |
| 5-3420 | A22 | J2 | Q250 |
| 5-3421 | A22 | J2 | Q251 |
| 5-3422 | A22 | J2 | Q256 |
| 5-3423 | A22 | J2 | Q258 |
| 5-3424 | A22 | J2 | Q259 |
| 5-3425 | A22 | J2 | Q260 |
| 5-3426 | A22 | J2 | Q261 |
| 5-3427 | A22 | J2 | Q262 |
| 5-3428 | A22 | J2 | Q263 |
| 5-3429 | A22 | J2 | Q264 |
| 5-3430 | A22 | J2 | Q266 |
| 5-3431 | A22 | J2 | Q267 |
| 5-3432 | A22 | J2 | Q268 |
| 5-3433 | A22 | J2 | Q269 |
| 5-3434 | A22 | J2 | Q270 |
| 5-3435 | A22 | J2 | Q271 |
| 5-3436 | A22 | J2 | Q272 |
| 5-3437 | A22 | J2 | Q274 |
| 5-3438 | A22 | J2 | Q275 |
| 5-3439 | A28 | J2 | Q1 |
| 5-3440 | A28 | J2 | Q2 |
| 5-3441 | A28 | J2 | Q3 |
| 5-3442 | A28 | J2 | Q4 |
| 5-3443 | A28 | J2 | Q5 |
| 5-3444 | A28 | J2 | Q6 |
| 5-3445 | A28 | J2 | Q7 |
| 5-3446 | A28 | J2 | Q8 |
| 5-3447 | A28 | J2 | Q9 |
| 5-3448 | A28 | J2 | Q10 |
| 5-3449 | A28 | J2 | Q11 |
| 5-3450 | A28 | J2 | Q12 |
| 5-3451 | A28 | J2 | Q13 |
| 5-3452 | A28 | J2 | Q14 |
| 5-3453 | A28 | J2 | Q15 |
| 5-3454 | A28 | J2 | Q16 |
| 5-3455 | A28 | J2 | Q17 |
| 5-3456 | A28 | J2 | Q18 |
| 5-3457 | A28 | J2 | Q19 |
| 5-3458 | A28 | J2 | Q20 |
| 5-3459 | A28 | J2 | Q21 |
| 5-3460 | A28 | J2 | Q23 |
| 5-3461 | A28 | J2 | Q24 |
| 5-3462 | A28 | J2 | Q25 |
| 5-3463 | A28 | J2 | Q26 |
| 5-3464 | A28 | J2 | Q27 |
| 5-3465 | A28 | J2 | Q28 |
| 5-3466 | A28 | J2 | Q29 |
| 5-3467 | A28 | J2 | Q30 |
| 5-3468 | A28 | J2 | Q31 |
| 5-3469 | A28 | J2 | Q32 |
| 5-3470 | A28 | J2 | Q33 |
| 5-3471 | A28 | J2 | Q34 |
| 5-3472 | A28 | J2 | Q35 |
| 5-3473 | A28 | J2 | Q36 |
| 5-3474 | A28 | J2 | Q37 |
| 5-3475 | A28 | J2 | Q38 |
| 5-3476 | A28 | J2 | Q39 |
| 5-3477 | A28 | J2 | Q40 |
| 5-3478 | A28 | J2 | Q41 |
| 5-3479 | A28 | J2 | Q42 |
| 5-3480 | A28 | J2 | Q43 |
| 5-3481 | A28 | J2 | Q44 |
| 5-3482 | A28 | J2 | Q45 |
| 5-3483 | A28 | J2 | Q46 |
| 5-3484 | A28 | J2 | Q47 |
| 5-3485 | A28 | J2 | Q48 |
| 5-3486 | A28 | J2 | Q49 |
| 5-3487 | A28 | J2 | Q50 |
| 5-3488 | A28 | J2 | Q51 |
| 5-3489 | A28 | J2 | Q53 |
| 5-3490 | A28 | J2 | Q54 |
| 5-3491 | A28 | J2 | Q55 |
| 5-3492 | A28 | J2 | Q57 |
| 5-3493 | A28 | J2 | Q60 |
| 5-3494 | A28 | J2 | Q61 |
| 5-3495 | A28 | J2 | Q62 |
| 5-3496 | A28 | J2 | Q63 |
| 5-3497 | A28 | J2 | Q64 |
| 5-3498 | A28 | J2 | Q67 |
| 5-3499 | A28 | J2 | Q68 |
| 5-3500 | A28 | J2 | Q69 |
| 5-3501 | A28 | J2 | Q71 |
| 5-3502 | A28 | J2 | Q72 |
| 5-3503 | A28 | J2 | Q73 |
| 5-3504 | A28 | J2 | Q74 |
| 5-3505 | A28 | J2 | Q75 |
| 5-3506 | A28 | J2 | Q76 |
| 5-3507 | A28 | J2 | Q77 |
| 5-3508 | A28 | J2 | Q82 |
| 5-3509 | A28 | J2 | Q83 |
| 5-3510 | A28 | J2 | Q84 |
| 5-3511 | A28 | J2 | Q85 |
| 5-3512 | A28 | J2 | Q86 |
| 5-3513 | A28 | J2 | Q87 |
| 5-3514 | A28 | J2 | Q88 |
| 5-3515 | A28 | J2 | Q89 |
| 5-3516 | A28 | J2 | Q90 |
| 5-3517 | A28 | J2 | Q91 |
| 5-3518 | A28 | J2 | Q92 |
| 5-3519 | A28 | J2 | Q93 |
| 5-3520 | A28 | J2 | Q94 |
| 5-3521 | A28 | J2 | Q95 |
| 5-3522 | A28 | J2 | Q96 |
| 5-3523 | A28 | J2 | Q97 |
| 5-3524 | A28 | J2 | Q99 |
| 5-3525 | A28 | J2 | Q100 |
| 5-3526 | A28 | J2 | Q101 |
| 5-3527 | A28 | J2 | Q102 |
| 5-3528 | A28 | J2 | Q103 |
| 5-3529 | A28 | J2 | Q104 |
| 5-3530 | A28 | J2 | Q105 |
| 5-3531 | A28 | J2 | Q107 |
| 5-3532 | A28 | J2 | Q108 |
| 5-3533 | A28 | J2 | Q109 |
| 5-3534 | A28 | J2 | Q110 |
| 5-3535 | A28 | J2 | Q111 |
| 5-3536 | A28 | J2 | Q112 |
| 5-3537 | A28 | J2 | Q113 |
| 5-3538 | A28 | J2 | Q114 |
| 5-3539 | A28 | J2 | Q115 |
| 5-3540 | A28 | J2 | Q116 |
| 5-3541 | A28 | J2 | Q117 |
| 5-3542 | A28 | J2 | Q118 |
| 5-3543 | A28 | J2 | Q119 |
| 5-3544 | A28 | J2 | Q120 |
| 5-3545 | A28 | J2 | Q121 |
| 5-3546 | A28 | J2 | Q122 |
| 5-3547 | A28 | J2 | Q126 |
| 5-3548 | A28 | J2 | Q127 |
| 5-3549 | A28 | J2 | Q128 |
| 5-3550 | A28 | J2 | Q129 |
| 5-3551 | A28 | J2 | Q130 |
| 5-3552 | A28 | J2 | Q131 |
| 5-3553 | A28 | J2 | Q132 |
| 5-3554 | A28 | J2 | Q133 |
| 5-3555 | A28 | J2 | Q134 |
| 5-3556 | A28 | J2 | Q135 |
| 5-3557 | A28 | J2 | Q136 |
| 5-3558 | A28 | J2 | Q137 |
| 5-3559 | A28 | J2 | Q138 |
| 5-3560 | A28 | J2 | Q139 |
| 5-3561 | A28 | J2 | Q140 |
| 5-3562 | A28 | J2 | Q143 |
| 5-3563 | A28 | J2 | Q144 |
| 5-3564 | A28 | J2 | Q145 |
| 5-3565 | A28 | J2 | Q146 |
| 5-3566 | A28 | J2 | Q148 |
| 5-3567 | A28 | J2 | Q149 |
| 5-3568 | A28 | J2 | Q153 |
| 5-3569 | A28 | J2 | Q155 |
| 5-3570 | A28 | J2 | Q156 |
| 5-3571 | A28 | J2 | Q157 |
| 5-3572 | A28 | J2 | Q158 |
| 5-3573 | A28 | J2 | Q159 |
| 5-3574 | A28 | J2 | Q161 |
| 5-3575 | A28 | J2 | Q162 |
| 5-3576 | A28 | J2 | Q163 |
| 5-3577 | A28 | J2 | Q164 |
| 5-3578 | A28 | J2 | Q165 |
| 5-3579 | A28 | J2 | Q166 |
| 5-3580 | A28 | J2 | Q167 |
| 5-3581 | A28 | J2 | Q168 |
| 5-3582 | A28 | J2 | Q169 |
| 5-3583 | A28 | J2 | Q170 |
| 5-3584 | A28 | J2 | Q171 |
| 5-3585 | A28 | J2 | Q172 |
| 5-3586 | A28 | J2 | Q173 |
| 5-3587 | A28 | J2 | Q174 |
| 5-3588 | A28 | J2 | Q175 |
| 5-3589 | A28 | J2 | Q176 |
| 5-3590 | A28 | J2 | Q177 |
| 5-3591 | A28 | J2 | Q178 |
| 5-3592 | A28 | J2 | Q179 |
| 5-3593 | A28 | J2 | Q181 |
| 5-3594 | A28 | J2 | Q182 |
| 5-3595 | A28 | J2 | Q183 |
| 5-3596 | A28 | J2 | Q184 |
| 5-3597 | A28 | J2 | Q185 |
| 5-3598 | A28 | J2 | Q186 |
| 5-3599 | A28 | J2 | Q187 |
| 5-3600 | A28 | J2 | Q188 |
| 5-3601 | A28 | J2 | Q189 |
| 5-3602 | A28 | J2 | Q190 |
| 5-3603 | A28 | J2 | Q191 |
| 5-3604 | A28 | J2 | Q192 |
| 5-3605 | A28 | J2 | Q193 |
| 5-3606 | A28 | J2 | Q194 |
| 5-3607 | A28 | J2 | Q195 |
| 5-3608 | A28 | J2 | Q196 |
| 5-3609 | A28 | J2 | Q197 |
| 5-3610 | A28 | J2 | Q198 |
| 5-3611 | A28 | J2 | Q199 |
| 5-3612 | A28 | J2 | Q200 |
| 5-3613 | A28 | J2 | Q201 |
| 5-3614 | A28 | J2 | Q202 |
| 5-3615 | A28 | J2 | Q203 |
| 5-3616 | A28 | J2 | Q204 |
| 5-3617 | A28 | J2 | Q205 |
| 5-3618 | A28 | J2 | Q206 |
| 5-3619 | A28 | J2 | Q207 |
| 5-3620 | A28 | J2 | Q208 |
| 5-3621 | A28 | J2 | Q209 |
| 5-3622 | A28 | J2 | Q210 |
| 5-3623 | A28 | J2 | Q211 |
| 5-3624 | A28 | J2 | Q212 |
| 5-3625 | A28 | J2 | Q213 |
| 5-3626 | A28 | J2 | Q214 |
| 5-3627 | A28 | J2 | Q215 |
| 5-3628 | A28 | J2 | Q216 |
| 5-3629 | A28 | J2 | Q217 |
| 5-3630 | A28 | J2 | Q222 |
| 5-3631 | A28 | J2 | Q223 |
| 5-3632 | A28 | J2 | Q224 |
| 5-3633 | A28 | J2 | Q225 |
| 5-3634 | A28 | J2 | Q226 |
| 5-3635 | A28 | J2 | Q227 |
| 5-3636 | A28 | J2 | Q228 |
| 5-3637 | A28 | J2 | Q229 |
| 5-3638 | A28 | J2 | Q230 |
| 5-3639 | A28 | J2 | Q231 |
| 5-3640 | A28 | J2 | Q232 |
| 5-3641 | A28 | J2 | Q233 |
| 5-3642 | A28 | J2 | Q234 |
| 5-3643 | A28 | J2 | Q235 |
| 5-3644 | A28 | J2 | Q236 |
| 5-3645 | A28 | J2 | Q237 |
| 5-3646 | A28 | J2 | Q238 |
| 5-3647 | A28 | J2 | Q239 |
| 5-3648 | A28 | J2 | Q240 |
| 5-3649 | A28 | J2 | Q241 |
| 5-3650 | A28 | J2 | Q242 |
| 5-3651 | A28 | J2 | Q243 |
| 5-3652 | A28 | J2 | Q244 |
| 5-3653 | A28 | J2 | Q246 |
| 5-3654 | A28 | J2 | Q247 |
| 5-3655 | A28 | J2 | Q249 |
| 5-3656 | A28 | J2 | Q250 |
| 5-3657 | A28 | J2 | Q251 |
| 5-3658 | A28 | J2 | Q256 |
| 5-3659 | A28 | J2 | Q258 |
| 5-3660 | A28 | J2 | Q259 |
| 5-3661 | A28 | J2 | Q260 |
| 5-3662 | A28 | J2 | Q261 |
| 5-3663 | A28 | J2 | Q262 |
| 5-3664 | A28 | J2 | Q263 |
| 5-3665 | A28 | J2 | Q264 |
| 5-3666 | A28 | J2 | Q266 |
| 5-3667 | A28 | J2 | Q267 |
| 5-3668 | A28 | J2 | Q268 |
| 5-3669 | A28 | J2 | Q269 |
| 5-3670 | A28 | J2 | Q270 |
| 5-3671 | A28 | J2 | Q271 |
| 5-3672 | A28 | J2 | Q272 |
| 5-3673 | A28 | J2 | Q274 |
| 5-3674 | A28 | J2 | Q275 |
| 5-3675 | A9 | J1 | Q1 |
| 5-3676 | A9 | J1 | Q2 |
| 5-3677 | A9 | J1 | Q3 |
| 5-3678 | A9 | J1 | Q4 |
| 5-3679 | A9 | J1 | Q5 |
| 5-3680 | A9 | J1 | Q6 |
| 5-3681 | A9 | J1 | Q7 |
| 5-3682 | A9 | J1 | Q8 |
| 5-3683 | A9 | J1 | Q9 |
| 5-3684 | A9 | J1 | Q10 |
| 5-3685 | A9 | J1 | Q11 |
| 5-3686 | A9 | J1 | Q12 |
| 5-3687 | A9 | J1 | Q13 |
| 5-3688 | A9 | J1 | Q14 |
| 5-3689 | A9 | J1 | Q15 |
| 5-3690 | A9 | J1 | Q16 |
| 5-3691 | A9 | J1 | Q17 |
| 5-3692 | A9 | J1 | Q18 |
| 5-3693 | A9 | J1 | Q19 |
| 5-3694 | A9 | J1 | Q20 |
| 5-3695 | A9 | J1 | Q21 |
| 5-3696 | A9 | J1 | Q23 |
| 5-3697 | A9 | J1 | Q24 |
| 5-3698 | A9 | J1 | Q25 |
| 5-3699 | A9 | J1 | Q26 |
| 5-3700 | A9 | J1 | Q27 |
| 5-3701 | A9 | J1 | Q28 |
| 5-3702 | A9 | J1 | Q29 |
| 5-3703 | A9 | J1 | Q30 |
| 5-3704 | A9 | J1 | Q31 |
| 5-3705 | A9 | J1 | Q32 |
| 5-3706 | A9 | J1 | Q33 |
| 5-3707 | A9 | J1 | Q34 |
| 5-3708 | A9 | J1 | Q35 |
| 5-3709 | A9 | J1 | Q36 |
| 5-3710 | A9 | J1 | Q37 |
| 5-3711 | A9 | J1 | Q38 |
| 5-3712 | A9 | J1 | Q39 |
| 5-3713 | A9 | J1 | Q40 |
| 5-3714 | A9 | J1 | Q41 |
| 5-3715 | A9 | J1 | Q42 |
| 5-3716 | A9 | J1 | Q43 |
| 5-3717 | A9 | J1 | Q44 |
| 5-3718 | A9 | J1 | Q45 |
| 5-3719 | A9 | J1 | Q46 |
| 5-3720 | A9 | J1 | Q47 |
| 5-3721 | A9 | J1 | Q48 |
| 5-3722 | A9 | J1 | Q49 |
| 5-3723 | A9 | J1 | Q50 |
| 5-3724 | A9 | J1 | Q51 |
| 5-3725 | A9 | J1 | Q53 |
| 5-3726 | A9 | J1 | Q54 |
| 5-3727 | A9 | J1 | Q55 |
| 5-3728 | A9 | J1 | Q57 |
| 5-3729 | A9 | J1 | Q60 |
| 5-3730 | A9 | J1 | Q61 |
| 5-3731 | A9 | J1 | Q62 |
| 5-3732 | A9 | J1 | Q63 |
| 5-3733 | A9 | J1 | Q64 |
| 5-3734 | A9 | J1 | Q67 |
| 5-3735 | A9 | J1 | Q68 |
| 5-3736 | A9 | J1 | Q69 |
| 5-3737 | A9 | J1 | Q71 |
| 5-3738 | A9 | J1 | Q72 |
| 5-3739 | A9 | J1 | Q73 |
| 5-3740 | A9 | J1 | Q74 |
| 5-3741 | A9 | J1 | Q75 |
| 5-3742 | A9 | J1 | Q76 |
| 5-3743 | A9 | J1 | Q77 |
| 5-3744 | A9 | J1 | Q82 |
| 5-3745 | A9 | J1 | Q83 |
| 5-3746 | A9 | J1 | Q84 |
| 5-3747 | A9 | J1 | Q85 |
| 5-3748 | A9 | J1 | Q86 |
| 5-3749 | A9 | J1 | Q87 |
| 5-3750 | A9 | J1 | Q88 |
| 5-3751 | A9 | J1 | Q89 |
| 5-3752 | A9 | J1 | Q90 |
| 5-3753 | A9 | J1 | Q91 |
| 5-3754 | A9 | J1 | Q92 |
| 5-3755 | A9 | J1 | Q93 |
| 5-3756 | A9 | J1 | Q94 |
| 5-3757 | A9 | J1 | Q95 |
| 5-3758 | A9 | J1 | Q96 |
| 5-3759 | A9 | J1 | Q97 |
| 5-3760 | A9 | J1 | Q99 |
| 5-3761 | A9 | J1 | Q100 |
| 5-3762 | A9 | J1 | Q101 |
| 5-3763 | A9 | J1 | Q102 |
| 5-3764 | A9 | J1 | Q103 |
| 5-3765 | A9 | J1 | Q104 |
| 5-3766 | A9 | J1 | Q105 |
| 5-3767 | A9 | J1 | Q107 |
| 5-3768 | A9 | J1 | Q108 |
| 5-3769 | A9 | J1 | Q109 |
| 5-3770 | A9 | J1 | Q110 |
| 5-3771 | A9 | J1 | Q111 |
| 5-3772 | A9 | J1 | Q112 |
| 5-3773 | A9 | J1 | Q113 |
| 5-3774 | A9 | J1 | Q114 |
| 5-3775 | A9 | J1 | Q115 |
| 5-3776 | A9 | J1 | Q116 |
| 5-3777 | A9 | J1 | Q117 |
| 5-3778 | A9 | J1 | Q118 |
| 5-3779 | A9 | J1 | Q119 |
| 5-3780 | A9 | J1 | Q120 |
| 5-3781 | A9 | J1 | Q121 |
| 5-3782 | A9 | J1 | Q122 |
| 5-3783 | A9 | J1 | Q126 |
| 5-3784 | A9 | J1 | Q127 |
| 5-3785 | A9 | J1 | Q128 |
| 5-3786 | A9 | J1 | Q129 |
| 5-3787 | A9 | J1 | Q130 |
| 5-3788 | A9 | J1 | Q131 |
| 5-3789 | A9 | J1 | Q132 |
| 5-3790 | A9 | J1 | Q133 |
| 5-3791 | A9 | J1 | Q134 |
| 5-3792 | A9 | J1 | Q135 |
| 5-3793 | A9 | J1 | Q136 |
| 5-3794 | A9 | J1 | Q137 |
| 5-3795 | A9 | J1 | Q138 |
| 5-3796 | A9 | J1 | Q139 |
| 5-3797 | A9 | J1 | Q140 |
| 5-3798 | A9 | J1 | Q143 |
| 5-3799 | A9 | J1 | Q144 |
| 5-3800 | A9 | J1 | Q145 |
| 5-3801 | A9 | J1 | Q146 |
| 5-3802 | A9 | J1 | Q148 |
| 5-3803 | A9 | J1 | Q149 |
| 5-3804 | A9 | J1 | Q153 |
| 5-3805 | A9 | J1 | Q155 |
| 5-3806 | A9 | J1 | Q156 |
| 5-3807 | A9 | J1 | Q157 |
| 5-3808 | A9 | J1 | Q158 |
| 5-3809 | A9 | J1 | Q159 |
| 5-3810 | A9 | J1 | Q161 |
| 5-3811 | A9 | J1 | Q162 |
| 5-3812 | A9 | J1 | Q163 |
| 5-3813 | A9 | J1 | Q164 |
| 5-3814 | A9 | J1 | Q165 |
| 5-3815 | A9 | J1 | Q166 |
| 5-3816 | A9 | J1 | Q167 |
| 5-3817 | A9 | J1 | Q168 |
| 5-3818 | A9 | J1 | Q169 |
| 5-3819 | A9 | J1 | Q170 |
| 5-3820 | A9 | J1 | Q171 |
| 5-3821 | A9 | J1 | Q172 |
| 5-3822 | A9 | J1 | Q173 |
| 5-3823 | A9 | J1 | Q174 |
| 5-3824 | A9 | J1 | Q175 |
| 5-3825 | A9 | J1 | Q176 |
| 5-3826 | A9 | J1 | Q177 |
| 5-3827 | A9 | J1 | Q178 |
| 5-3828 | A9 | J1 | Q179 |
| 5-3829 | A9 | J1 | Q181 |
| 5-3830 | A9 | J1 | Q182 |
| 5-3831 | A9 | J1 | Q183 |
| 5-3832 | A9 | J1 | Q184 |
| 5-3833 | A9 | J1 | Q185 |
| 5-3834 | A9 | J1 | Q186 |
| 5-3835 | A9 | J1 | Q187 |
| 5-3836 | A9 | J1 | Q188 |
| 5-3837 | A9 | J1 | Q189 |
| 5-3838 | A9 | J1 | Q190 |
| 5-3839 | A9 | J1 | Q191 |
| 5-3840 | A9 | J1 | Q192 |
| 5-3841 | A9 | J1 | Q193 |
| 5-3842 | A9 | J1 | Q194 |
| 5-3843 | A9 | J1 | Q195 |
| 5-3844 | A9 | J1 | Q196 |
| 5-3845 | A9 | J1 | Q197 |
| 5-3846 | A9 | J1 | Q198 |
| 5-3847 | A9 | J1 | Q199 |
| 5-3848 | A9 | J1 | Q200 |
| 5-3849 | A9 | J1 | Q201 |
| 5-3850 | A9 | J1 | Q202 |
| 5-3851 | A9 | J1 | Q203 |
| 5-3852 | A9 | J1 | Q204 |
| 5-3853 | A9 | J1 | Q205 |
| 5-3854 | A9 | J1 | Q206 |
| 5-3855 | A9 | J1 | Q207 |
| 5-3856 | A9 | J1 | Q208 |
| 5-3857 | A9 | J1 | Q209 |
| 5-3858 | A9 | J1 | Q210 |
| 5-3859 | A9 | J1 | Q211 |
| 5-3860 | A9 | J1 | Q212 |
| 5-3861 | A9 | J1 | Q213 |
| 5-3862 | A9 | J1 | Q214 |
| 5-3863 | A9 | J1 | Q215 |
| 5-3864 | A9 | J1 | Q216 |
| 5-3865 | A9 | J1 | Q217 |
| 5-3866 | A9 | J1 | Q222 |
| 5-3867 | A9 | J1 | Q223 |
| 5-3868 | A9 | J1 | Q224 |
| 5-3869 | A9 | J1 | Q225 |
| 5-3870 | A9 | J1 | Q226 |
| 5-3871 | A9 | J1 | Q227 |
| 5-3872 | A9 | J1 | Q228 |
| 5-3873 | A9 | J1 | Q229 |
| 5-3874 | A9 | J1 | Q230 |
| 5-3875 | A9 | J1 | Q231 |
| 5-3876 | A9 | J1 | Q232 |
| 5-3877 | A9 | J1 | Q233 |
| 5-3878 | A9 | J1 | Q234 |
| 5-3879 | A9 | J1 | Q235 |
| 5-3880 | A9 | J1 | Q236 |
| 5-3881 | A9 | J1 | Q237 |
| 5-3882 | A9 | J1 | Q238 |
| 5-3883 | A9 | J1 | Q239 |
| 5-3884 | A9 | J1 | Q240 |
| 5-3885 | A9 | J1 | Q241 |
| 5-3886 | A9 | J1 | Q242 |
| 5-3887 | A9 | J1 | Q243 |
| 5-3888 | A9 | J1 | Q244 |
| 5-3889 | A9 | J1 | Q246 |
| 5-3890 | A9 | J1 | Q247 |
| 5-3891 | A9 | J1 | Q249 |
| 5-3892 | A9 | J1 | Q250 |
| 5-3893 | A9 | J1 | Q251 |
| 5-3894 | A9 | J1 | Q256 |
| 5-3895 | A9 | J1 | Q258 |
| 5-3896 | A9 | J1 | Q259 |
| 5-3897 | A9 | J1 | Q260 |
| 5-3898 | A9 | J1 | Q261 |
| 5-3899 | A9 | J1 | Q262 |
| 5-3900 | A9 | J1 | Q263 |
| 5-3901 | A9 | J1 | Q264 |
| 5-3902 | A9 | J1 | Q266 |
| 5-3903 | A9 | J1 | Q267 |
| 5-3904 | A9 | J1 | Q268 |
| 5-3905 | A9 | J1 | Q269 |
| 5-3906 | A9 | J1 | Q270 |
| 5-3907 | A9 | J1 | Q271 |
| 5-3908 | A9 | J1 | Q272 |
| 5-3909 | A9 | J1 | Q274 |
| 5-3910 | A9 | J1 | Q275 |
| 5-3911 | A21 | J1 | Q1 |
| 5-3912 | A21 | J1 | Q2 |
| 5-3913 | A21 | J1 | Q3 |
| 5-3914 | A21 | J1 | Q4 |
| 5-3915 | A21 | J1 | Q5 |
| 5-3916 | A21 | J1 | Q6 |
| 5-3917 | A21 | J1 | Q7 |
| 5-3918 | A21 | J1 | Q8 |
| 5-3919 | A21 | J1 | Q9 |
| 5-3920 | A21 | J1 | Q10 |
| 5-3921 | A21 | J1 | Q11 |
| 5-3922 | A21 | J1 | Q12 |
| 5-3923 | A21 | J1 | Q13 |
| 5-3924 | A21 | J1 | Q14 |
| 5-3925 | A21 | J1 | Q15 |
| 5-3926 | A21 | J1 | Q16 |
| 5-3927 | A21 | J1 | Q17 |
| 5-3928 | A21 | J1 | Q18 |
| 5-3929 | A21 | J1 | Q19 |
| 5-3930 | A21 | J1 | Q20 |
| 5-3931 | A21 | J1 | Q21 |
| 5-3932 | A21 | J1 | Q23 |
| 5-3933 | A21 | J1 | Q24 |
| 5-3934 | A21 | J1 | Q25 |
| 5-3935 | A21 | J1 | Q26 |
| 5-3936 | A21 | J1 | Q27 |
| 5-3937 | A21 | J1 | Q28 |
| 5-3938 | A21 | J1 | Q29 |
| 5-3939 | A21 | J1 | Q30 |
| 5-3940 | A21 | J1 | Q31 |
| 5-3941 | A21 | J1 | Q32 |
| 5-3942 | A21 | J1 | Q33 |
| 5-3943 | A21 | J1 | Q34 |
| 5-3944 | A21 | J1 | Q35 |
| 5-3945 | A21 | J1 | Q36 |
| 5-3946 | A21 | J1 | Q37 |
| 5-3947 | A21 | J1 | Q38 |
| 5-3948 | A21 | J1 | Q39 |
| 5-3949 | A21 | J1 | Q40 |
| 5-3950 | A21 | J1 | Q41 |
| 5-3951 | A21 | J1 | Q42 |
| 5-3952 | A21 | J1 | Q43 |
| 5-3953 | A21 | J1 | Q44 |
| 5-3954 | A21 | J1 | Q45 |
| 5-3955 | A21 | J1 | Q46 |
| 5-3956 | A21 | J1 | Q47 |
| 5-3957 | A21 | J1 | Q48 |
| 5-3958 | A21 | J1 | Q49 |
| 5-3959 | A21 | J1 | Q50 |
| 5-3960 | A21 | J1 | Q51 |
| 5-3961 | A21 | J1 | Q53 |
| 5-3962 | A21 | J1 | Q54 |
| 5-3963 | A21 | J1 | Q55 |
| 5-3964 | A21 | J1 | Q57 |
| 5-3965 | A21 | J1 | Q60 |
| 5-3966 | A21 | J1 | Q61 |
| 5-3967 | A21 | J1 | Q62 |
| 5-3968 | A21 | J1 | Q63 |
| 5-3969 | A21 | J1 | Q64 |
| 5-3970 | A21 | J1 | Q67 |
| 5-3971 | A21 | J1 | Q68 |
| 5-3972 | A21 | J1 | Q69 |
| 5-3973 | A21 | J1 | Q71 |
| 5-3974 | A21 | J1 | Q72 |
| 5-3975 | A21 | J1 | Q73 |
| 5-3976 | A21 | J1 | Q74 |
| 5-3977 | A21 | J1 | Q75 |
| 5-3978 | A21 | J1 | Q76 |
| 5-3979 | A21 | J1 | Q77 |
| 5-3980 | A21 | J1 | Q82 |
| 5-3981 | A21 | J1 | Q83 |
| 5-3982 | A21 | J1 | Q84 |
| 5-3983 | A21 | J1 | Q85 |
| 5-3984 | A21 | J1 | Q86 |
| 5-3985 | A21 | J1 | Q87 |
| 5-3986 | A21 | J1 | Q88 |
| 5-3987 | A21 | J1 | Q89 |
| 5-3988 | A21 | J1 | Q90 |
| 5-3989 | A21 | J1 | Q91 |
| 5-3990 | A21 | J1 | Q92 |
| 5-3991 | A21 | J1 | Q93 |
| 5-3992 | A21 | J1 | Q94 |
| 5-3993 | A21 | J1 | Q95 |
| 5-3994 | A21 | J1 | Q96 |
| 5-3995 | A21 | J1 | Q97 |
| 5-3996 | A21 | J1 | Q99 |
| 5-3997 | A21 | J1 | Q100 |
| 5-3998 | A21 | J1 | Q101 |
| 5-3999 | A21 | J1 | Q102 |
| 5-4000 | A21 | J1 | Q103 |
| 5-4001 | A21 | J1 | Q104 |
| 5-4002 | A21 | J1 | Q105 |
| 5-4003 | A21 | J1 | Q107 |
| 5-4004 | A21 | J1 | Q108 |
| 5-4005 | A21 | J1 | Q109 |
| 5-4006 | A21 | J1 | Q110 |
| 5-4007 | A21 | J1 | Q111 |
| 5-4008 | A21 | J1 | Q112 |
| 5-4009 | A21 | J1 | Q113 |
| 5-4010 | A21 | J1 | Q114 |
| 5-4011 | A21 | J1 | Q115 |
| 5-4012 | A21 | J1 | Q116 |
| 5-4013 | A21 | J1 | Q117 |
| 5-4014 | A21 | J1 | Q118 |
| 5-4015 | A21 | J1 | Q119 |
| 5-4016 | A21 | J1 | Q120 |
| 5-4017 | A21 | J1 | Q121 |
| 5-4018 | A21 | J1 | Q122 |
| 5-4019 | A21 | J1 | Q126 |
| 5-4020 | A21 | J1 | Q127 |
| 5-4021 | A21 | J1 | Q128 |
| 5-4022 | A21 | J1 | Q129 |
| 5-4023 | A21 | J1 | Q130 |
| 5-4024 | A21 | J1 | Q131 |
| 5-4025 | A21 | J1 | Q132 |
| 5-4026 | A21 | J1 | Q133 |
| 5-4027 | A21 | J1 | Q134 |
| 5-4028 | A21 | J1 | Q135 |
| 5-4029 | A21 | J1 | Q136 |
| 5-4030 | A21 | J1 | Q137 |
| 5-4031 | A21 | J1 | Q138 |
| 5-4032 | A21 | J1 | Q139 |
| 5-4033 | A21 | J1 | Q140 |
| 5-4034 | A21 | J1 | Q143 |
| 5-4035 | A21 | J1 | Q144 |
| 5-4036 | A21 | J1 | Q145 |
| 5-4037 | A21 | J1 | Q146 |
| 5-4038 | A21 | J1 | Q148 |
| 5-4039 | A21 | J1 | Q149 |
| 5-4040 | A21 | J1 | Q153 |
| 5-4041 | A21 | J1 | Q155 |
| 5-4042 | A21 | J1 | Q156 |
| 5-4043 | A21 | J1 | Q157 |
| 5-4044 | A21 | J1 | Q158 |
| 5-4045 | A21 | J1 | Q159 |
| 5-4046 | A21 | J1 | Q161 |
| 5-4047 | A21 | J1 | Q162 |
| 5-4048 | A21 | J1 | Q163 |
| 5-4049 | A21 | J1 | Q164 |
| 5-4050 | A21 | J1 | Q165 |
| 5-4051 | A21 | J1 | Q166 |
| 5-4052 | A21 | J1 | Q167 |
| 5-4053 | A21 | J1 | Q168 |
| 5-4054 | A21 | J1 | Q169 |
| 5-4055 | A21 | J1 | Q170 |
| 5-4056 | A21 | J1 | Q171 |
| 5-4057 | A21 | J1 | Q172 |
| 5-4058 | A21 | J1 | Q173 |
| 5-4059 | A21 | J1 | Q174 |
| 5-4060 | A21 | J1 | Q175 |
| 5-4061 | A21 | J1 | Q176 |
| 5-4062 | A21 | J1 | Q177 |
| 5-4063 | A21 | J1 | Q178 |
| 5-4064 | A21 | J1 | Q179 |
| 5-4065 | A21 | J1 | Q181 |
| 5-4066 | A21 | J1 | Q182 |
| 5-4067 | A21 | J1 | Q183 |
| 5-4068 | A21 | J1 | Q184 |
| 5-4069 | A21 | J1 | Q185 |
| 5-4070 | A21 | J1 | Q186 |
| 5-4071 | A21 | J1 | Q187 |
| 5-4072 | A21 | J1 | Q188 |
| 5-4073 | A21 | J1 | Q189 |
| 5-4074 | A21 | J1 | Q190 |
| 5-4075 | A21 | J1 | Q191 |
| 5-4076 | A21 | J1 | Q192 |
| 5-4077 | A21 | J1 | Q193 |
| 5-4078 | A21 | J1 | Q194 |
| 5-4079 | A21 | J1 | Q195 |
| 5-4080 | A21 | J1 | Q196 |
| 5-4081 | A21 | J1 | Q197 |
| 5-4082 | A21 | J1 | Q198 |
| 5-4083 | A21 | J1 | Q199 |
| 5-4084 | A21 | J1 | Q200 |
| 5-4085 | A21 | J1 | Q201 |
| 5-4086 | A21 | J1 | Q202 |
| 5-4087 | A21 | J1 | Q203 |
| 5-4088 | A21 | J1 | Q204 |
| 5-4089 | A21 | J1 | Q205 |
| 5-4090 | A21 | J1 | Q206 |
| 5-4091 | A21 | J1 | Q207 |
| 5-4092 | A21 | J1 | Q208 |
| 5-4093 | A21 | J1 | Q209 |
| 5-4094 | A21 | J1 | Q210 |
| 5-4095 | A21 | J1 | Q211 |
| 5-4096 | A21 | J1 | Q212 |
| 5-4097 | A21 | J1 | Q213 |
| 5-4098 | A21 | J1 | Q214 |
| 5-4099 | A21 | J1 | Q215 |
| 5-4100 | A21 | J1 | Q216 |
| 5-4101 | A21 | J1 | Q217 |
| 5-4102 | A21 | J1 | Q222 |
| 5-4103 | A21 | J1 | Q223 |
| 5-4104 | A21 | J1 | Q224 |
| 5-4105 | A21 | J1 | Q225 |
| 5-4106 | A21 | J1 | Q226 |
| 5-4107 | A21 | J1 | Q227 |
| 5-4108 | A21 | J1 | Q228 |
| 5-4109 | A21 | J1 | Q229 |
| 5-4110 | A21 | J1 | Q230 |
| 5-4111 | A21 | J1 | Q231 |
| 5-4112 | A21 | J1 | Q232 |
| 5-4113 | A21 | J1 | Q233 |
| 5-4114 | A21 | J1 | Q234 |
| 5-4115 | A21 | J1 | Q235 |
| 5-4116 | A21 | J1 | Q236 |
| 5-4117 | A21 | J1 | Q237 |
| 5-4118 | A21 | J1 | Q238 |
| 5-4119 | A21 | J1 | Q239 |
| 5-4120 | A21 | J1 | Q240 |
| 5-4121 | A21 | J1 | Q241 |
| 5-4122 | A21 | J1 | Q242 |
| 5-4123 | A21 | J1 | Q243 |
| 5-4124 | A21 | J1 | Q244 |
| 5-4125 | A21 | J1 | Q246 |
| 5-4126 | A21 | J1 | Q247 |
| 5-4127 | A21 | J1 | Q249 |
| 5-4128 | A21 | J1 | Q250 |
| 5-4129 | A21 | J1 | Q251 |
| 5-4130 | A21 | J1 | Q256 |
| 5-4131 | A21 | J1 | Q258 |
| 5-4132 | A21 | J1 | Q259 |
| 5-4133 | A21 | J1 | Q260 |
| 5-4134 | A21 | J1 | Q261 |
| 5-4135 | A21 | J1 | Q262 |
| 5-4136 | A21 | J1 | Q263 |
| 5-4137 | A21 | J1 | Q264 |
| 5-4138 | A21 | J1 | Q266 |
| 5-4139 | A21 | J1 | Q267 |
| 5-4140 | A21 | J1 | Q268 |
| 5-4141 | A21 | J1 | Q269 |
| 5-4142 | A21 | J1 | Q270 |
| 5-4143 | A21 | J1 | Q271 |
| 5-4144 | A21 | J1 | Q272 |
| 5-4145 | A21 | J1 | Q274 |
| 5-4146 | A21 | J1 | Q275 |
| 5-4147 | A22 | J1 | Q1 |
| 5-4148 | A22 | J1 | Q2 |
| 5-4149 | A22 | J1 | Q3 |
| 5-4150 | A22 | J1 | Q4 |
| 5-4151 | A22 | J1 | Q5 |
| 5-4152 | A22 | J1 | Q6 |
| 5-4153 | A22 | J1 | Q7 |
| 5-4154 | A22 | J1 | Q8 |
| 5-4155 | A22 | J1 | Q9 |
| 5-4156 | A22 | J1 | Q10 |
| 5-4157 | A22 | J1 | Q11 |
| 5-4158 | A22 | J1 | Q12 |
| 5-4159 | A22 | J1 | Q13 |
| 5-4160 | A22 | J1 | Q14 |
| 5-4161 | A22 | J1 | Q15 |
| 5-4162 | A22 | J1 | Q16 |
| 5-4163 | A22 | J1 | Q17 |
| 5-4164 | A22 | J1 | Q18 |
| 5-4165 | A22 | J1 | Q19 |
| 5-4166 | A22 | J1 | Q20 |
| 5-4167 | A22 | J1 | Q21 |
| 5-4168 | A22 | J1 | Q23 |
| 5-4169 | A22 | J1 | Q24 |
| 5-4170 | A22 | J1 | Q25 |
| 5-4171 | A22 | J1 | Q26 |
| 5-4172 | A22 | J1 | Q27 |
| 5-4173 | A22 | J1 | Q28 |
| 5-4174 | A22 | J1 | Q29 |
| 5-4175 | A22 | J1 | Q30 |
| 5-4176 | A22 | J1 | Q31 |
| 5-4177 | A22 | J1 | Q32 |
| 5-4178 | A22 | J1 | Q33 |
| 5-4179 | A22 | J1 | Q34 |
| 5-4180 | A22 | J1 | Q35 |
| 5-4181 | A22 | J1 | Q36 |
| 5-4182 | A22 | J1 | Q37 |
| 5-4183 | A22 | J1 | Q38 |
| 5-4184 | A22 | J1 | Q39 |
| 5-4185 | A22 | J1 | Q40 |
| 5-4186 | A22 | J1 | Q41 |
| 5-4187 | A22 | J1 | Q42 |
| 5-4188 | A22 | J1 | Q43 |
| 5-4189 | A22 | J1 | Q44 |
| 5-4190 | A22 | J1 | Q45 |
| 5-4191 | A22 | J1 | Q46 |
| 5-4192 | A22 | J1 | Q47 |
| 5-4193 | A22 | J1 | Q48 |
| 5-4194 | A22 | J1 | Q49 |
| 5-4195 | A22 | J1 | Q50 |
| 5-4196 | A22 | J1 | Q51 |
| 5-4197 | A22 | J1 | Q53 |
| 5-4198 | A22 | J1 | Q54 |
| 5-4199 | A22 | J1 | Q55 |
| 5-4200 | A22 | J1 | Q57 |
| 5-4201 | A22 | J1 | Q60 |
| 5-4202 | A22 | J1 | Q61 |
| 5-4203 | A22 | J1 | Q62 |
| 5-4204 | A22 | J1 | Q63 |
| 5-4205 | A22 | J1 | Q64 |
| 5-4206 | A22 | J1 | Q67 |
| 5-4207 | A22 | J1 | Q68 |
| 5-4208 | A22 | J1 | Q69 |
| 5-4209 | A22 | J1 | Q71 |
| 5-4210 | A22 | J1 | Q72 |
| 5-4211 | A22 | J1 | Q73 |
| 5-4212 | A22 | J1 | Q74 |
| 5-4213 | A22 | J1 | Q75 |
| 5-4214 | A22 | J1 | Q76 |
| 5-4215 | A22 | J1 | Q77 |
| 5-4216 | A22 | J1 | Q82 |
| 5-4217 | A22 | J1 | Q83 |
| 5-4218 | A22 | J1 | Q84 |
| 5-4219 | A22 | J1 | Q85 |
| 5-4220 | A22 | J1 | Q86 |
| 5-4221 | A22 | J1 | Q87 |
| 5-4222 | A22 | J1 | Q88 |
| 5-4223 | A22 | J1 | Q89 |
| 5-4224 | A22 | J1 | Q90 |
| 5-4225 | A22 | J1 | Q91 |
| 5-4226 | A22 | J1 | Q92 |
| 5-4227 | A22 | J1 | Q93 |
| 5-4228 | A22 | J1 | Q94 |
| 5-4229 | A22 | J1 | Q95 |
| 5-4230 | A22 | J1 | Q96 |
| 5-4231 | A22 | J1 | Q97 |
| 5-4232 | A22 | J1 | Q99 |
| 5-4233 | A22 | J1 | Q100 |
| 5-4234 | A22 | J1 | Q101 |
| 5-4235 | A22 | J1 | Q102 |
| 5-4236 | A22 | J1 | Q103 |
| 5-4237 | A22 | J1 | Q104 |
| 5-4238 | A22 | J1 | Q105 |
| 5-4239 | A22 | J1 | Q107 |
| 5-4240 | A22 | J1 | Q108 |
| 5-4241 | A22 | J1 | Q109 |
| 5-4242 | A22 | J1 | Q110 |
| 5-4243 | A22 | J1 | Q111 |
| 5-4244 | A22 | J1 | Q112 |
| 5-4245 | A22 | J1 | Q113 |
| 5-4246 | A22 | J1 | Q114 |
| 5-4247 | A22 | J1 | Q115 |
| 5-4248 | A22 | J1 | Q116 |
| 5-4249 | A22 | J1 | Q117 |
| 5-4250 | A22 | J1 | Q118 |
| 5-4251 | A22 | J1 | Q119 |
| 5-4252 | A22 | J1 | Q120 |
| 5-4253 | A22 | J1 | Q121 |
| 5-4254 | A22 | J1 | Q122 |
| 5-4255 | A22 | J1 | Q126 |
| 5-4256 | A22 | J1 | Q127 |
| 5-4257 | A22 | J1 | Q128 |
| 5-4258 | A22 | J1 | Q129 |
| 5-4259 | A22 | J1 | Q130 |
| 5-4260 | A22 | J1 | Q131 |
| 5-4261 | A22 | J1 | Q132 |
| 5-4262 | A22 | J1 | Q133 |
| 5-4263 | A22 | J1 | Q134 |
| 5-4264 | A22 | J1 | Q135 |
| 5-4265 | A22 | J1 | Q136 |
| 5-4266 | A22 | J1 | Q137 |
| 5-4267 | A22 | J1 | Q138 |
| 5-4268 | A22 | J1 | Q139 |
| 5-4269 | A22 | J1 | Q140 |
| 5-4270 | A22 | J1 | Q143 |
| 5-4271 | A22 | J1 | Q144 |
| 5-4272 | A22 | J1 | Q145 |
| 5-4273 | A22 | J1 | Q146 |
| 5-4274 | A22 | J1 | Q148 |
| 5-4275 | A22 | J1 | Q149 |
| 5-4276 | A22 | J1 | Q153 |
| 5-4277 | A22 | J1 | Q155 |
| 5-4278 | A22 | J1 | Q156 |
| 5-4279 | A22 | J1 | Q157 |
| 5-4280 | A22 | J1 | Q158 |
| 5-4281 | A22 | J1 | Q159 |
| 5-4282 | A22 | J1 | Q161 |
| 5-4283 | A22 | J1 | Q162 |
| 5-4284 | A22 | J1 | Q163 |
| 5-4285 | A22 | J1 | Q164 |
| 5-4286 | A22 | J1 | Q165 |
| 5-4287 | A22 | J1 | Q166 |
| 5-4288 | A22 | J1 | Q167 |
| 5-4289 | A22 | J1 | Q168 |
| 5-4290 | A22 | J1 | Q169 |
| 5-4291 | A22 | J1 | Q170 |
| 5-4292 | A22 | J1 | Q171 |
| 5-4293 | A22 | J1 | Q172 |
| 5-4294 | A22 | J1 | Q173 |
| 5-4295 | A22 | J1 | Q174 |
| 5-4296 | A22 | J1 | Q175 |
| 5-4297 | A22 | J1 | Q176 |
| 5-4298 | A22 | J1 | Q177 |
| 5-4299 | A22 | J1 | Q178 |
| 5-4300 | A22 | J1 | Q179 |
| 5-4301 | A22 | J1 | Q181 |
| 5-4302 | A22 | J1 | Q182 |
| 5-4303 | A22 | J1 | Q183 |
| 5-4304 | A22 | J1 | Q184 |
| 5-4305 | A22 | J1 | Q185 |
| 5-4306 | A22 | J1 | Q186 |
| 5-4307 | A22 | J1 | Q187 |
| 5-4308 | A22 | J1 | Q188 |
| 5-4309 | A22 | J1 | Q189 |
| 5-4310 | A22 | J1 | Q190 |
| 5-4311 | A22 | J1 | Q191 |
| 5-4312 | A22 | J1 | Q192 |
| 5-4313 | A22 | J1 | Q193 |
| 5-4314 | A22 | J1 | Q194 |
| 5-4315 | A22 | J1 | Q195 |
| 5-4316 | A22 | J1 | Q196 |
| 5-4317 | A22 | J1 | Q197 |
| 5-4318 | A22 | J1 | Q198 |
| 5-4319 | A22 | J1 | Q199 |
| 5-4320 | A22 | J1 | Q200 |
| 5-4321 | A22 | J1 | Q201 |
| 5-4322 | A22 | J1 | Q202 |
| 5-4323 | A22 | J1 | Q203 |
| 5-4324 | A22 | J1 | Q204 |
| 5-4325 | A22 | J1 | Q205 |
| 5-4326 | A22 | J1 | Q206 |
| 5-4327 | A22 | J1 | Q207 |
| 5-4328 | A22 | J1 | Q208 |
| 5-4329 | A22 | J1 | Q209 |
| 5-4330 | A22 | J1 | Q210 |
| 5-4331 | A22 | J1 | Q211 |
| 5-4332 | A22 | J1 | Q212 |
| 5-4333 | A22 | J1 | Q213 |
| 5-4334 | A22 | J1 | Q214 |
| 5-4335 | A22 | J1 | Q215 |
| 5-4336 | A22 | J1 | Q216 |
| 5-4337 | A22 | J1 | Q217 |
| 5-4338 | A22 | J1 | Q222 |
| 5-4339 | A22 | J1 | Q223 |
| 5-4340 | A22 | J1 | Q224 |
| 5-4341 | A22 | J1 | Q225 |
| 5-4342 | A22 | J1 | Q226 |
| 5-4343 | A22 | J1 | Q227 |
| 5-4344 | A22 | J1 | Q228 |
| 5-4345 | A22 | J1 | Q229 |
| 5-4346 | A22 | J1 | Q230 |
| 5-4347 | A22 | J1 | Q231 |
| 5-4348 | A22 | J1 | Q232 |
| 5-4349 | A22 | J1 | Q233 |
| 5-4350 | A22 | J1 | Q234 |
| 5-4351 | A22 | J1 | Q235 |
| 5-4352 | A22 | J1 | Q236 |
| 5-4353 | A22 | J1 | Q237 |
| 5-4354 | A22 | J1 | Q238 |
| 5-4355 | A22 | J1 | Q239 |
| 5-4356 | A22 | J1 | Q240 |
| 5-4357 | A22 | J1 | Q241 |
| 5-4358 | A22 | J1 | Q242 |
| 5-4359 | A22 | J1 | Q243 |
| 5-4360 | A22 | J1 | Q244 |
| 5-4361 | A22 | J1 | Q246 |
| 5-4362 | A22 | J1 | Q247 |
| 5-4363 | A22 | J1 | Q249 |
| 5-4364 | A22 | J1 | Q250 |
| 5-4365 | A22 | J1 | Q251 |
| 5-4366 | A22 | J1 | Q256 |
| 5-4367 | A22 | J1 | Q258 |
| 5-4368 | A22 | J1 | Q259 |
| 5-4369 | A22 | J1 | Q260 |
| 5-4370 | A22 | J1 | Q261 |
| 5-4371 | A22 | J1 | Q262 |
| 5-4372 | A22 | J1 | Q263 |
| 5-4373 | A22 | J1 | Q264 |
| 5-4374 | A22 | J1 | Q266 |
| 5-4375 | A22 | J1 | Q267 |
| 5-4376 | A22 | J1 | Q268 |
| 5-4377 | A22 | J1 | Q269 |
| 5-4378 | A22 | J1 | Q270 |
| 5-4379 | A22 | J1 | Q271 |
| 5-4380 | A22 | J1 | Q272 |
| 5-4381 | A22 | J1 | Q274 |
| 5-4382 | A22 | J1 | Q275 |
| 5-4383 | A28 | J1 | Q1 |
| 5-4384 | A28 | J1 | Q2 |
| 5-4385 | A28 | J1 | Q3 |
| 5-4386 | A28 | J1 | Q4 |
| 5-4387 | A28 | J1 | Q5 |
| 5-4388 | A28 | J1 | Q6 |
| 5-4389 | A28 | J1 | Q7 |
| 5-4390 | A28 | J1 | Q8 |
| 5-4391 | A28 | J1 | Q9 |
| 5-4392 | A28 | J1 | Q10 |
| 5-4393 | A28 | J1 | Q11 |
| 5-4394 | A28 | J1 | Q12 |
| 5-4395 | A28 | J1 | Q13 |
| 5-4396 | A28 | J1 | Q14 |
| 5-4397 | A28 | J1 | Q15 |
| 5-4398 | A28 | J1 | Q16 |
| 5-4399 | A28 | J1 | Q17 |
| 5-4400 | A28 | J1 | Q18 |
| 5-4401 | A28 | J1 | Q19 |
| 5-4402 | A28 | J1 | Q20 |
| 5-4403 | A28 | J1 | Q21 |
| 5-4404 | A28 | J1 | Q23 |
| 5-4405 | A28 | J1 | Q24 |
| 5-4406 | A28 | J1 | Q25 |
| 5-4407 | A28 | J1 | Q26 |
| 5-4408 | A28 | J1 | Q27 |
| 5-4409 | A28 | J1 | Q28 |
| 5-4410 | A28 | J1 | Q29 |
| 5-4411 | A28 | J1 | Q30 |
| 5-4412 | A28 | J1 | Q31 |
| 5-4413 | A28 | J1 | Q32 |
| 5-4414 | A28 | J1 | Q33 |
| 5-4415 | A28 | J1 | Q34 |
| 5-4416 | A28 | J1 | Q35 |
| 5-4417 | A28 | J1 | Q36 |
| 5-4418 | A28 | J1 | Q37 |
| 5-4419 | A28 | J1 | Q38 |
| 5-4420 | A28 | J1 | Q39 |
| 5-4421 | A28 | J1 | Q40 |
| 5-4422 | A28 | J1 | Q41 |
| 5-4423 | A28 | J1 | Q42 |
| 5-4424 | A28 | J1 | Q43 |
| 5-4425 | A28 | J1 | Q44 |
| 5-4426 | A28 | J1 | Q45 |
| 5-4427 | A28 | J1 | Q46 |
| 5-4428 | A28 | J1 | Q47 |
| 5-4429 | A28 | J1 | Q48 |
| 5-4430 | A28 | J1 | Q49 |
| 5-4431 | A28 | J1 | Q50 |
| 5-4432 | A28 | J1 | Q51 |
| 5-4433 | A28 | J1 | Q53 |
| 5-4434 | A28 | J1 | Q54 |
| 5-4435 | A28 | J1 | Q55 |
| 5-4436 | A28 | J1 | Q57 |
| 5-4437 | A28 | J1 | Q60 |
| 5-4438 | A28 | J1 | Q61 |
| 5-4439 | A28 | J1 | Q62 |
| 5-4440 | A28 | J1 | Q63 |
| 5-4441 | A28 | J1 | Q64 |
| 5-4442 | A28 | J1 | Q67 |
| 5-4443 | A28 | J1 | Q68 |
| 5-4444 | A28 | J1 | Q69 |
| 5-4445 | A28 | J1 | Q71 |
| 5-4446 | A28 | J1 | Q72 |
| 5-4447 | A28 | J1 | Q73 |
| 5-4448 | A28 | J1 | Q74 |
| 5-4449 | A28 | J1 | Q75 |
| 5-4450 | A28 | J1 | Q76 |
| 5-4451 | A28 | J1 | Q77 |
| 5-4452 | A28 | J1 | Q82 |
| 5-4453 | A28 | J1 | Q83 |
| 5-4454 | A28 | J1 | Q84 |
| 5-4455 | A28 | J1 | Q85 |
| 5-4456 | A28 | J1 | Q86 |
| 5-4457 | A28 | J1 | Q87 |
| 5-4458 | A28 | J1 | Q88 |
| 5-4459 | A28 | J1 | Q89 |
| 5-4460 | A28 | J1 | Q90 |
| 5-4461 | A28 | J1 | Q91 |
| 5-4462 | A28 | J1 | Q92 |
| 5-4463 | A28 | J1 | Q93 |
| 5-4464 | A28 | J1 | Q94 |
| 5-4465 | A28 | J1 | Q95 |
| 5-4466 | A28 | J1 | Q96 |
| 5-4467 | A28 | J1 | Q97 |
| 5-4468 | A28 | J1 | Q99 |
| 5-4469 | A28 | J1 | Q100 |
| 5-4470 | A28 | J1 | Q101 |
| 5-4471 | A28 | J1 | Q102 |
| 5-4472 | A28 | J1 | Q103 |
| 5-4473 | A28 | J1 | Q104 |
| 5-4474 | A28 | J1 | Q105 |
| 5-4475 | A28 | J1 | Q107 |
| 5-4476 | A28 | J1 | Q108 |
| 5-4477 | A28 | J1 | Q109 |
| 5-4478 | A28 | J1 | Q110 |
| 5-4479 | A28 | J1 | Q111 |
| 5-4480 | A28 | J1 | Q112 |
| 5-4481 | A28 | J1 | Q113 |
| 5-4482 | A28 | J1 | Q114 |
| 5-4483 | A28 | J1 | Q115 |
| 5-4484 | A28 | J1 | Q116 |
| 5-4485 | A28 | J1 | Q117 |
| 5-4486 | A28 | J1 | Q118 |
| 5-4487 | A28 | J1 | Q119 |
| 5-4488 | A28 | J1 | Q120 |
| 5-4489 | A28 | J1 | Q121 |
| 5-4490 | A28 | J1 | Q122 |
| 5-4491 | A28 | J1 | Q126 |
| 5-4492 | A28 | J1 | Q127 |
| 5-4493 | A28 | J1 | Q128 |
| 5-4494 | A28 | J1 | Q129 |
| 5-4495 | A28 | J1 | Q130 |
| 5-4496 | A28 | J1 | Q131 |
| 5-4497 | A28 | J1 | Q132 |
| 5-4498 | A28 | J1 | Q133 |
| 5-4499 | A28 | J1 | Q134 |
| 5-4500 | A28 | J1 | Q135 |
| 5-4501 | A28 | J1 | Q136 |
| 5-4502 | A28 | J1 | Q137 |
| 5-4503 | A28 | J1 | Q138 |
| 5-4504 | A28 | J1 | Q139 |
| 5-4505 | A28 | J1 | Q140 |
| 5-4506 | A28 | J1 | Q143 |
| 5-4507 | A28 | J1 | Q144 |
| 5-4508 | A28 | J1 | Q145 |
| 5-4509 | A28 | J1 | Q146 |
| 5-4510 | A28 | J1 | Q148 |
| 5-4511 | A28 | J1 | Q149 |
| 5-4512 | A28 | J1 | Q153 |
| 5-4513 | A28 | J1 | Q155 |
| 5-4514 | A28 | J1 | Q156 |
| 5-4515 | A28 | J1 | Q157 |
| 5-4516 | A28 | J1 | Q158 |
| 5-4517 | A28 | J1 | Q159 |
| 5-4518 | A28 | J1 | Q161 |
| 5-4519 | A28 | J1 | Q162 |
| 5-4520 | A28 | J1 | Q163 |
| 5-4521 | A28 | J1 | Q164 |
| 5-4522 | A28 | J1 | Q165 |
| 5-4523 | A28 | J1 | Q166 |
| 5-4524 | A28 | J1 | Q167 |
| 5-4525 | A28 | J1 | Q168 |
| 5-4526 | A28 | J1 | Q169 |
| 5-4527 | A28 | J1 | Q170 |
| 5-4528 | A28 | J1 | Q171 |
| 5-4529 | A28 | J1 | Q172 |
| 5-4530 | A28 | J1 | Q173 |
| 5-4531 | A28 | J1 | Q174 |
| 5-4532 | A28 | J1 | Q175 |
| 5-4533 | A28 | J1 | Q176 |
| 5-4534 | A28 | J1 | Q177 |
| 5-4535 | A28 | J1 | Q178 |
| 5-4536 | A28 | J1 | Q179 |
| 5-4537 | A28 | J1 | Q181 |
| 5-4538 | A28 | J1 | Q182 |
| 5-4539 | A28 | J1 | Q183 |
| 5-4540 | A28 | J1 | Q184 |
| 5-4541 | A28 | J1 | Q185 |
| 5-4542 | A28 | J1 | Q186 |
| 5-4543 | A28 | J1 | Q187 |
| 5-4544 | A28 | J1 | Q188 |
| 5-4545 | A28 | J1 | Q189 |
| 5-4546 | A28 | J1 | Q190 |
| 5-4547 | A28 | J1 | Q191 |
| 5-4548 | A28 | J1 | Q192 |
| 5-4549 | A28 | J1 | Q193 |
| 5-4550 | A28 | J1 | Q194 |
| 5-4551 | A28 | J1 | Q195 |
| 5-4552 | A28 | J1 | Q196 |
| 5-4553 | A28 | J1 | Q197 |
| 5-4554 | A28 | J1 | Q198 |
| 5-4555 | A28 | J1 | Q199 |
| 5-4556 | A28 | J1 | Q200 |
| 5-4557 | A28 | J1 | Q201 |
| 5-4558 | A28 | J1 | Q202 |
| 5-4559 | A28 | J1 | Q203 |
| 5-4560 | A28 | J1 | Q204 |
| 5-4561 | A28 | J1 | Q205 |
| 5-4562 | A28 | J1 | Q206 |
| 5-4563 | A28 | J1 | Q207 |
| 5-4564 | A28 | J1 | Q208 |
| 5-4565 | A28 | J1 | Q209 |
| 5-4566 | A28 | J1 | Q210 |
| 5-4567 | A28 | J1 | Q211 |
| 5-4568 | A28 | J1 | Q212 |
| 5-4569 | A28 | J1 | Q213 |
| 5-4570 | A28 | J1 | Q214 |
| 5-4571 | A28 | J1 | Q215 |
| 5-4572 | A28 | J1 | Q216 |
| 5-4573 | A28 | J1 | Q217 |
| 5-4574 | A28 | J1 | Q222 |
| 5-4575 | A28 | J1 | Q223 |
| 5-4576 | A28 | J1 | Q224 |
| 5-4577 | A28 | J1 | Q225 |
| 5-4578 | A28 | J1 | Q226 |
| 5-4579 | A28 | J1 | Q227 |
| 5-4580 | A28 | J1 | Q228 |
| 5-4581 | A28 | J1 | Q229 |
| 5-4582 | A28 | J1 | Q230 |
| 5-4583 | A28 | J1 | Q231 |
| 5-4584 | A28 | J1 | Q232 |
| 5-4585 | A28 | J1 | Q233 |
| 5-4586 | A28 | J1 | Q234 |
| 5-4587 | A28 | J1 | Q235 |
| 5-4588 | A28 | J1 | Q236 |
| 5-4589 | A28 | J1 | Q237 |
| 5-4590 | A28 | J1 | Q238 |
| 5-4591 | A28 | J1 | Q239 |
| 5-4592 | A28 | J1 | Q240 |
| 5-4593 | A28 | J1 | Q241 |
| 5-4594 | A28 | J1 | Q242 |
| 5-4595 | A28 | J1 | Q243 |
| 5-4596 | A28 | J1 | Q244 |
| 5-4597 | A28 | J1 | Q246 |
| 5-4598 | A28 | J1 | Q247 |
| 5-4599 | A28 | J1 | Q249 |
| 5-4600 | A28 | J1 | Q250 |
| 5-4601 | A28 | J1 | Q251 |
| 5-4602 | A28 | J1 | Q256 |
| 5-4603 | A28 | J1 | Q258 |
| 5-4604 | A28 | J1 | Q259 |
| 5-4605 | A28 | J1 | Q260 |
| 5-4606 | A28 | J1 | Q261 |
| 5-4607 | A28 | J1 | Q262 |
| 5-4608 | A28 | J1 | Q263 |
| 5-4609 | A28 | J1 | Q264 |
| 5-4610 | A28 | J1 | Q266 |
| 5-4611 | A28 | J1 | Q267 |
| 5-4612 | A28 | J1 | Q268 |
| 5-4613 | A28 | J1 | Q269 |
| 5-4614 | A28 | J1 | Q270 |
| 5-4615 | A28 | J1 | Q271 |
| 5-4616 | A28 | J1 | Q272 |
| 5-4617 | A28 | J1 | Q274 |
| 5-4618 | A28 | J1 | Q275 |
| 5-4619 | A21 | J3 | Q65 |
| 5-4620 | A21 | J4 | Q65 |
| 5-4621 | A21 | J5 | Q65 |
| 5-4622 | A21 | J6 | Q65 |
| 5-4623 | A21 | J7 | Q65 |
| 5-4624 | A21 | J8 | Q65 |
| 5-4625 | A21 | J9 | Q65 |
| 5-4626 | A21 | J10 | Q65 |
| 5-4627 | A21 | J11 | Q65 |
| 5-4628 | A21 | J12 | Q65 |
| 5-4629 | A21 | J13 | Q65 |
| 5-4630 | A21 | J14 | Q65 |
| 5-4631 | A21 | J15 | Q65 |
| 5-4632 | A21 | J16 | Q65 |
| 5-4633 | A21 | J17 | Q65 |
| 5-4634 | A21 | J18 | Q65 |
| 5-4635 | A21 | J19 | Q65 |
| 5-4636 | A21 | J20 | Q65 |
| 5-4637 | A21 | J21 | Q65 |
| 5-4638 | A21 | J22 | Q65 |
| 5-4639 | A21 | J23 | Q65 |
| 5-4640 | A21 | J24 | Q65 |
| 5-4641 | A21 | J25 | Q65 |
| 5-4642 | A21 | J26 | Q65 |
| 5-4643 | A21 | J27 | Q65 |
| 5-4644 | A21 | J28 | Q65 |
| 5-4645 | A21 | J29 | Q65 |
| 5-4646 | A21 | J30 | Q65 |
| 5-4647 | A21 | J31 | Q65 |
| 5-4648 | A21 | J32 | Q65 |
| 5-4649 | A21 | J33 | Q65 |
| 5-4650 | A21 | J34 | Q65 |
| 5-4651 | A21 | J35 | Q65 |
| 5-4652 | A21 | J36 | Q65 |
| 5-4653 | A21 | J37 | Q65 |
| 5-4654 | A21 | J38 | Q65 |
| 5-4655 | A21 | J39 | Q65 |
| 5-4656 | A21 | J40 | Q65 |
| 5-4657 | A21 | J41 | Q65 |
| 5-4658 | A21 | J42 | Q65 |
| 5-4659 | A21 | J43 | Q65 |
| 5-4660 | A21 | J44 | Q65 |
| 5-4661 | A21 | J45 | Q65 |
| 5-4662 | A21 | J46 | Q65 |
| 5-4663 | A21 | J47 | Q65 |
| 5-4664 | A21 | J48 | Q65 |
| 5-4665 | A21 | J49 | Q65 |
| 5-4666 | A21 | J50 | Q65 |
| 5-4667 | A21 | J51 | Q65 |
| 5-4668 | A21 | J52 | Q65 |
| 5-4669 | A21 | J53 | Q65 |
| 5-4670 | A21 | J54 | Q65 |
| 5-4671 | A21 | J55 | Q65 |
| 5-4672 | A21 | J56 | Q65 |
| 5-4673 | A21 | J3 | Q106 |
| 5-4674 | A21 | J4 | Q106 |
| 5-4675 | A21 | J5 | Q106 |
| 5-4676 | A21 | J6 | Q106 |
| 5-4677 | A21 | J7 | Q106 |
| 5-4678 | A21 | J8 | Q106 |
| 5-4679 | A21 | J9 | Q106 |
| 5-4680 | A21 | J10 | Q106 |
| 5-4681 | A21 | J11 | Q106 |
| 5-4682 | A21 | J12 | Q106 |
| 5-4683 | A21 | J13 | Q106 |
| 5-4684 | A21 | J14 | Q106 |
| 5-4685 | A21 | J15 | Q106 |
| 5-4686 | A21 | J16 | Q106 |
| 5-4687 | A21 | J17 | Q106 |
| 5-4688 | A21 | J18 | Q106 |
| 5-4689 | A21 | J19 | Q106 |
| 5-4690 | A21 | J20 | Q106 |
| 5-4691 | A21 | J21 | Q106 |
| 5-4692 | A21 | J22 | Q106 |
| 5-4693 | A21 | J23 | Q106 |
| 5-4694 | A21 | J24 | Q106 |
| 5-4695 | A21 | J25 | Q106 |
| 5-4696 | A21 | J26 | Q106 |
| 5-4697 | A21 | J27 | Q106 |
| 5-4698 | A21 | J28 | Q106 |
| 5-4699 | A21 | J29 | Q106 |
| 5-4700 | A21 | J30 | Q106 |
| 5-4701 | A21 | J31 | Q106 |
| 5-4702 | A21 | J32 | Q106 |
| 5-4703 | A21 | J33 | Q106 |
| 5-4704 | A21 | J34 | Q106 |
| 5-4705 | A21 | J35 | Q106 |
| 5-4706 | A21 | J36 | Q106 |
| 5-4707 | A21 | J37 | Q106 |
| 5-4708 | A21 | J38 | Q106 |
| 5-4709 | A21 | J39 | Q106 |
| 5-4710 | A21 | J40 | Q106 |
| 5-4711 | A21 | J41 | Q106 |
| 5-4712 | A21 | J42 | Q106 |
| 5-4713 | A21 | J43 | Q106 |
| 5-4714 | A21 | J44 | Q106 |
| 5-4715 | A21 | J45 | Q106 |
| 5-4716 | A21 | J46 | Q106 |
| 5-4717 | A21 | J47 | Q106 |
| 5-4718 | A21 | J48 | Q106 |
| 5-4719 | A21 | J49 | Q106 |
| 5-4720 | A21 | J50 | Q106 |
| 5-4721 | A21 | J51 | Q106 |
| 5-4722 | A21 | J52 | Q106 |
| 5-4723 | A21 | J53 | Q106 |
| 5-4724 | A21 | J54 | Q106 |
| 5-4725 | A21 | J55 | Q106 |
| 5-4726 | A21 | J56 | Q106 |
| 5-4727 | A21 | J3 | Q125 |
| 5-4728 | A21 | J4 | Q125 |
| 5-4729 | A21 | J5 | Q125 |
| 5-4730 | A21 | J6 | Q125 |
| 5-4731 | A21 | J7 | Q125 |
| 5-4732 | A21 | J8 | Q125 |
| 5-4733 | A21 | J9 | Q125 |
| 5-4734 | A21 | J10 | Q125 |
| 5-4735 | A21 | J11 | Q125 |
| 5-4736 | A21 | J12 | Q125 |
| 5-4737 | A21 | J13 | Q125 |
| 5-4738 | A21 | J14 | Q125 |
| 5-4739 | A21 | J15 | Q125 |
| 5-4740 | A21 | J16 | Q125 |
| 5-4741 | A21 | J17 | Q125 |
| 5-4742 | A21 | J18 | Q125 |
| 5-4743 | A21 | J19 | Q125 |
| 5-4744 | A21 | J20 | Q125 |
| 5-4745 | A21 | J21 | Q125 |
| 5-4746 | A21 | J22 | Q125 |
| 5-4747 | A21 | J23 | Q125 |
| 5-4748 | A21 | J24 | Q125 |
| 5-4749 | A21 | J25 | Q125 |
| 5-4750 | A21 | J26 | Q125 |
| 5-4751 | A21 | J27 | Q125 |
| 5-4752 | A21 | J28 | Q125 |
| 5-4753 | A21 | J29 | Q125 |
| 5-4754 | A21 | J30 | Q125 |
| 5-4755 | A21 | J31 | Q125 |
| 5-4756 | A21 | J32 | Q125 |
| 5-4757 | A21 | J33 | Q125 |
| 5-4758 | A21 | J34 | Q125 |
| 5-4759 | A21 | J35 | Q125 |
| 5-4760 | A21 | J36 | Q125 |
| 5-4761 | A21 | J37 | Q125 |
| 5-4762 | A21 | J38 | Q125 |
| 5-4763 | A21 | J39 | Q125 |
| 5-4764 | A21 | J40 | Q125 |
| 5-4765 | A21 | J41 | Q125 |
| 5-4766 | A21 | J42 | Q125 |
| 5-4767 | A21 | J43 | Q125 |
| 5-4768 | A21 | J44 | Q125 |
| 5-4769 | A21 | J45 | Q125 |
| 5-4770 | A21 | J46 | Q125 |
| 5-4771 | A21 | J47 | Q125 |
| 5-4772 | A21 | J48 | Q125 |
| 5-4773 | A21 | J49 | Q125 |
| 5-4774 | A21 | J50 | Q125 |
| 5-4775 | A21 | J51 | Q125 |
| 5-4776 | A21 | J52 | Q125 |
| 5-4777 | A21 | J53 | Q125 |
| 5-4778 | A21 | J54 | Q125 |
| 5-4779 | A21 | J55 | Q125 |
| 5-4780 | A21 | J56 | Q125 |
| 5-4781 | A22 | J3 | Q65 |
| 5-4782 | A22 | J4 | Q65 |
| 5-4783 | A22 | J5 | Q65 |
| 5-4784 | A22 | J6 | Q65 |
| 5-4785 | A22 | J7 | Q65 |
| 5-4786 | A22 | J8 | Q65 |
| 5-4787 | A22 | J9 | Q65 |
| 5-4788 | A22 | J10 | Q65 |
| 5-4789 | A22 | J11 | Q65 |
| 5-4790 | A22 | J12 | Q65 |
| 5-4791 | A22 | J13 | Q65 |
| 5-4792 | A22 | J14 | Q65 |
| 5-4793 | A22 | J15 | Q65 |
| 5-4794 | A22 | J16 | Q65 |
| 5-4795 | A22 | J17 | Q65 |
| 5-4796 | A22 | J18 | Q65 |
| 5-4797 | A22 | J19 | Q65 |
| 5-4798 | A22 | J20 | Q65 |
| 5-4799 | A22 | J21 | Q65 |
| 5-4800 | A22 | J22 | Q65 |
| 5-4801 | A22 | J23 | Q65 |
| 5-4802 | A22 | J24 | Q65 |
| 5-4803 | A22 | J25 | Q65 |
| 5-4804 | A22 | J26 | Q65 |
| 5-4805 | A22 | J27 | Q65 |
| 5-4806 | A22 | J28 | Q65 |
| 5-4807 | A22 | J29 | Q65 |
| 5-4808 | A22 | J30 | Q65 |
| 5-4809 | A22 | J31 | Q65 |
| 5-4810 | A22 | J32 | Q65 |
| 5-4811 | A22 | J33 | Q65 |
| 5-4812 | A22 | J34 | Q65 |
| 5-4813 | A22 | J35 | Q65 |
| 5-4814 | A22 | J36 | Q65 |
| 5-4815 | A22 | J37 | Q65 |
| 5-4816 | A22 | J38 | Q65 |
| 5-4817 | A22 | J39 | Q65 |
| 5-4818 | A22 | J40 | Q65 |
| 5-4819 | A22 | J41 | Q65 |
| 5-4820 | A22 | J42 | Q65 |
| 5-4821 | A22 | J43 | Q65 |
| 5-4822 | A22 | J44 | Q65 |
| 5-4823 | A22 | J45 | Q65 |
| 5-4824 | A22 | J46 | Q65 |
| 5-4825 | A22 | J47 | Q65 |
| 5-4826 | A22 | J48 | Q65 |
| 5-4827 | A22 | J49 | Q65 |
| 5-4828 | A22 | J50 | Q65 |
| 5-4829 | A22 | J51 | Q65 |
| 5-4830 | A22 | J52 | Q65 |
| 5-4831 | A22 | J53 | Q65 |
| 5-4832 | A22 | J54 | Q65 |
| 5-4833 | A22 | J55 | Q65 |
| 5-4834 | A22 | J56 | Q65 |
| 5-4835 | A22 | J3 | Q106 |
| 5-4836 | A22 | J4 | Q106 |
| 5-4837 | A22 | J5 | Q106 |
| 5-4838 | A22 | J6 | Q106 |
| 5-4839 | A22 | J7 | Q106 |
| 5-4840 | A22 | J8 | Q106 |
| 5-4841 | A22 | J9 | Q106 |
| 5-4842 | A22 | J10 | Q106 |
| 5-4843 | A22 | J11 | Q106 |
| 5-4844 | A22 | J12 | Q106 |
| 5-4845 | A22 | J13 | Q106 |
| 5-4846 | A22 | J14 | Q106 |
| 5-4847 | A22 | J15 | Q106 |
| 5-4848 | A22 | J16 | Q106 |
| 5-4849 | A22 | J17 | Q106 |
| 5-4850 | A22 | J18 | Q106 |
| 5-4851 | A22 | J19 | Q106 |
| 5-4852 | A22 | J20 | Q106 |
| 5-4853 | A22 | J21 | Q106 |
| 5-4854 | A22 | J22 | Q106 |
| 5-4855 | A22 | J23 | Q106 |
| 5-4856 | A22 | J24 | Q106 |
| 5-4857 | A22 | J25 | Q106 |
| 5-4858 | A22 | J26 | Q106 |
| 5-4859 | A22 | J27 | Q106 |
| 5-4860 | A22 | J28 | Q106 |
| 5-4861 | A22 | J29 | Q106 |
| 5-4862 | A22 | J30 | Q106 |
| 5-4863 | A22 | J31 | Q106 |
| 5-4864 | A22 | J32 | Q106 |
| 5-4865 | A22 | J33 | Q106 |
| 5-4866 | A22 | J34 | Q106 |
| 5-4867 | A22 | J35 | Q106 |
| 5-4868 | A22 | J36 | Q106 |
| 5-4869 | A22 | J37 | Q106 |
| 5-4870 | A22 | J38 | Q106 |
| 5-4871 | A22 | J39 | Q106 |
| 5-4872 | A22 | J40 | Q106 |
| 5-4873 | A22 | J41 | Q106 |
| 5-4874 | A22 | J42 | Q106 |
| 5-4875 | A22 | J43 | Q106 |
| 5-4876 | A22 | J44 | Q106 |
| 5-4877 | A22 | J45 | Q106 |
| 5-4878 | A22 | J46 | Q106 |
| 5-4879 | A22 | J47 | Q106 |
| 5-4880 | A22 | J48 | Q106 |
| 5-4881 | A22 | J49 | Q106 |
| 5-4882 | A22 | J50 | Q106 |
| 5-4883 | A22 | J51 | Q106 |
| 5-4884 | A22 | J52 | Q106 |
| 5-4885 | A22 | J53 | Q106 |
| 5-4886 | A22 | J54 | Q106 |
| 5-4887 | A22 | J55 | Q106 |
| 5-4888 | A22 | J56 | Q106 |
| 5-4889 | A22 | J3 | Q125 |
| 5-4890 | A22 | J4 | Q125 |
| 5-4891 | A22 | J5 | Q125 |
| 5-4892 | A22 | J6 | Q125 |
| 5-4893 | A22 | J7 | Q125 |
| 5-4894 | A22 | J8 | Q125 |
| 5-4895 | A22 | J9 | Q125 |
| 5-4896 | A22 | J10 | Q125 |
| 5-4897 | A22 | J11 | Q125 |
| 5-4898 | A22 | J12 | Q125 |
| 5-4899 | A22 | J13 | Q125 |
| 5-4900 | A22 | J14 | Q125 |
| 5-4901 | A22 | J15 | Q125 |
| 5-4902 | A22 | J16 | Q125 |
| 5-4903 | A22 | J17 | Q125 |
| 5-4904 | A22 | J18 | Q125 |
| 5-4905 | A22 | J19 | Q125 |
| 5-4906 | A22 | J20 | Q125 |
| 5-4907 | A22 | J21 | Q125 |
| 5-4908 | A22 | J22 | Q125 |
| 5-4909 | A22 | J23 | Q125 |
| 5-4910 | A22 | J24 | Q125 |
| 5-4911 | A22 | J25 | Q125 |
| 5-4912 | A22 | J26 | Q125 |
| 5-4913 | A22 | J27 | Q125 |
| 5-4914 | A22 | J28 | Q125 |
| 5-4915 | A22 | J29 | Q125 |
| 5-4916 | A22 | J30 | Q125 |
| 5-4917 | A22 | J31 | Q125 |
| 5-4918 | A22 | J32 | Q125 |
| 5-4919 | A22 | J33 | Q125 |
| 5-4920 | A22 | J34 | Q125 |
| 5-4921 | A22 | J35 | Q125 |
| 5-4922 | A22 | J36 | Q125 |
| 5-4923 | A22 | J37 | Q125 |
| 5-4924 | A22 | J38 | Q125 |
| 5-4925 | A22 | J39 | Q125 |
| 5-4926 | A22 | J40 | Q125 |
| 5-4927 | A22 | J41 | Q125 |
| 5-4928 | A22 | J42 | Q125 |
| 5-4929 | A22 | J43 | Q125 |
| 5-4930 | A22 | J44 | Q125 |
| 5-4931 | A22 | J45 | Q125 |
| 5-4932 | A22 | J46 | Q125 |
| 5-4933 | A22 | J47 | Q125 |
| 5-4934 | A22 | J48 | Q125 |
| 5-4935 | A22 | J49 | Q125 |
| 5-4936 | A22 | J50 | Q125 |
| 5-4937 | A22 | J51 | Q125 |
| 5-4938 | A22 | J52 | Q125 |
| 5-4939 | A22 | J53 | Q125 |
| 5-4940 | A22 | J54 | Q125 |
| 5-4941 | A22 | J55 | Q125 |
| 5-4942 | A22 | J56 | Q125 |
| 5-4943 | A28 | J3 | Q65 |
| 5-4944 | A28 | J4 | Q65 |
| 5-4945 | A28 | J5 | Q65 |
| 5-4946 | A28 | J6 | Q65 |
| 5-4947 | A28 | J7 | Q65 |
| 5-4948 | A28 | J8 | Q65 |
| 5-4949 | A28 | J9 | Q65 |
| 5-4950 | A28 | J10 | Q65 |
| 5-4951 | A28 | J11 | Q65 |
| 5-4952 | A28 | J12 | Q65 |
| 5-4953 | A28 | J13 | Q65 |
| 5-4954 | A28 | J14 | Q65 |
| 5-4955 | A28 | J15 | Q65 |
| 5-4956 | A28 | J16 | Q65 |
| 5-4957 | A28 | J17 | Q65 |
| 5-4958 | A28 | J18 | Q65 |
| 5-4959 | A28 | J19 | Q65 |
| 5-4960 | A28 | J20 | Q65 |
| 5-4961 | A28 | J21 | Q65 |
| 5-4962 | A28 | J22 | Q65 |
| 5-4963 | A28 | J23 | Q65 |
| 5-4964 | A28 | J24 | Q65 |
| 5-4965 | A28 | J25 | Q65 |
| 5-4966 | A28 | J26 | Q65 |
| 5-4967 | A28 | J27 | Q65 |
| 5-4968 | A28 | J28 | Q65 |
| 5-4969 | A28 | J29 | Q65 |
| 5-4970 | A28 | J30 | Q65 |
| 5-4971 | A28 | J31 | Q65 |
| 5-4972 | A28 | J32 | Q65 |
| 5-4973 | A28 | J33 | Q65 |
| 5-4974 | A28 | J34 | Q65 |
| 5-4975 | A28 | J35 | Q65 |
| 5-4976 | A28 | J36 | Q65 |
| 5-4977 | A28 | J37 | Q65 |
| 5-4978 | A28 | J38 | Q65 |
| 5-4979 | A28 | J39 | Q65 |
| 5-4980 | A28 | J40 | Q65 |
| 5-4981 | A28 | J41 | Q65 |
| 5-4982 | A28 | J42 | Q65 |
| 5-4983 | A28 | J43 | Q65 |
| 5-4984 | A28 | J44 | Q65 |
| 5-4985 | A28 | J45 | Q65 |
| 5-4986 | A28 | J46 | Q65 |
| 5-4987 | A28 | J47 | Q65 |
| 5-4988 | A28 | J48 | Q65 |
| 5-4989 | A28 | J49 | Q65 |
| 5-4990 | A28 | J50 | Q65 |
| 5-4991 | A28 | J51 | Q65 |
| 5-4992 | A28 | J52 | Q65 |
| 5-4993 | A28 | J53 | Q65 |
| 5-4994 | A28 | J54 | Q65 |
| 5-4995 | A28 | J55 | Q65 |
| 5-4996 | A28 | J56 | Q65 |
| 5-4997 | A28 | J3 | Q106 |
| 5-4998 | A28 | J4 | Q106 |
| 5-4999 | A28 | J5 | Q106 |
| 5-5000 | A28 | J6 | Q106 |
| 5-5001 | A28 | J7 | Q106 |
| 5-5002 | A28 | J8 | Q106 |
| 5-5003 | A28 | J9 | Q106 |
| 5-5004 | A28 | J10 | Q106 |
| 5-5005 | A28 | J11 | Q106 |
| 5-5006 | A28 | J12 | Q106 |
| 5-5007 | A28 | J13 | Q106 |
| 5-5008 | A28 | J14 | Q106 |
| 5-5009 | A28 | J15 | Q106 |
| 5-5010 | A28 | J16 | Q106 |
| 5-5011 | A28 | J17 | Q106 |
| 5-5012 | A28 | J18 | Q106 |
| 5-5013 | A28 | J19 | Q106 |
| 5-5014 | A28 | J20 | Q106 |
| 5-5015 | A28 | J21 | Q106 |
| 5-5016 | A28 | J22 | Q106 |
| 5-5017 | A28 | J23 | Q106 |
| 5-5018 | A28 | J24 | Q106 |
| 5-5019 | A28 | J25 | Q106 |
| 5-5020 | A28 | J26 | Q106 |
| 5-5021 | A28 | J27 | Q106 |
| 5-5022 | A28 | J28 | Q106 |
| 5-5023 | A28 | J29 | Q106 |
| 5-5024 | A28 | J30 | Q106 |
| 5-5025 | A28 | J31 | Q106 |
| 5-5026 | A28 | J32 | Q106 |
| 5-5027 | A28 | J33 | Q106 |
| 5-5028 | A28 | J34 | Q106 |
| 5-5029 | A28 | J35 | Q106 |
| 5-5030 | A28 | J36 | Q106 |
| 5-5031 | A28 | J37 | Q106 |
| 5-5032 | A28 | J38 | Q106 |
| 5-5033 | A28 | J39 | Q106 |
| 5-5034 | A28 | J40 | Q106 |
| 5-5035 | A28 | J41 | Q106 |
| 5-5036 | A28 | J42 | Q106 |
| 5-5037 | A28 | J43 | Q106 |
| 5-5038 | A28 | J44 | Q106 |
| 5-5039 | A28 | J45 | Q106 |
| 5-5040 | A28 | J46 | Q106 |
| 5-5041 | A28 | J47 | Q106 |
| 5-5042 | A28 | J48 | Q106 |
| 5-5043 | A28 | J49 | Q106 |
| 5-5044 | A28 | J50 | Q106 |
| 5-5045 | A28 | J51 | Q106 |
| 5-5046 | A28 | J52 | Q106 |
| 5-5047 | A28 | J53 | Q106 |
| 5-5048 | A28 | J54 | Q106 |
| 5-5049 | A28 | J55 | Q106 |
| 5-5050 | A28 | J56 | Q106 |
| 5-5051 | A28 | J3 | Q125 |
| 5-5052 | A28 | J4 | Q125 |
| 5-5053 | A28 | J5 | Q125 |
| 5-5054 | A28 | J6 | Q125 |
| 5-5055 | A28 | J7 | Q125 |
| 5-5056 | A28 | J8 | Q125 |
| 5-5057 | A28 | J9 | Q125 |
| 5-5058 | A28 | J10 | Q125 |
| 5-5059 | A28 | J11 | Q125 |
| 5-5060 | A28 | J12 | Q125 |
| 5-5061 | A28 | J13 | Q125 |
| 5-5062 | A28 | J14 | Q125 |
| 5-5063 | A28 | J15 | Q125 |
| 5-5064 | A28 | J16 | Q125 |
| 5-5065 | A28 | J17 | Q125 |
| 5-5066 | A28 | J18 | Q125 |
| 5-5067 | A28 | J19 | Q125 |
| 5-5068 | A28 | J20 | Q125 |
| 5-5069 | A28 | J21 | Q125 |
| 5-5070 | A28 | J22 | Q125 |
| 5-5071 | A28 | J23 | Q125 |
| 5-5072 | A28 | J24 | Q125 |
| 5-5073 | A28 | J25 | Q125 |
| 5-5074 | A28 | J26 | Q125 |
| 5-5075 | A28 | J27 | Q125 |
| 5-5076 | A28 | J28 | Q125 |
| 5-5077 | A28 | J29 | Q125 |
| 5-5078 | A28 | J30 | Q125 |
| 5-5079 | A28 | J31 | Q125 |
| 5-5080 | A28 | J32 | Q125 |
| 5-5081 | A28 | J33 | Q125 |
| 5-5082 | A28 | J34 | Q125 |
| 5-5083 | A28 | J35 | Q125 |
| 5-5084 | A28 | J36 | Q125 |
| 5-5085 | A28 | J37 | Q125 |
| 5-5086 | A28 | J38 | Q125 |
| 5-5087 | A28 | J39 | Q125 |
| 5-5088 | A28 | J40 | Q125 |
| 5-5089 | A28 | J41 | Q125 |
| 5-5090 | A28 | J42 | Q125 |
| 5-5091 | A28 | J43 | Q125 |
| 5-5092 | A28 | J44 | Q125 |
| 5-5093 | A28 | J45 | Q125 |
| 5-5094 | A28 | J46 | Q125 |
| 5-5095 | A28 | J47 | Q125 |
| 5-5096 | A28 | J48 | Q125 |
| 5-5097 | A28 | J49 | Q125 |
| 5-5098 | A28 | J50 | Q125 |
| 5-5099 | A28 | J51 | Q125 |
| 5-5100 | A28 | J52 | Q125 |
| 5-5101 | A28 | J53 | Q125 |
| 5-5102 | A28 | J54 | Q125 |
| 5-5103 | A28 | J55 | Q125 |
| 5-5104 | A28 | J56 | Q125 |

For compounds of the formula (I) according to the invention obtained in crystalline form, the melting points were measured. The melting points (° C.) for some exemplary compounds of Table 5 are listed below:

compound 5-183: 102° C.
compound 5-186: 160° C.
compound 5-225: 201-203° C.
compound 5-246: 197-199° C.
compound 5-435: 202-204° C.
compound 5-561: 166-168° C.
compound 5-582: 172-174° C.
compound 5-603: 217-219° C.
compound 5-1002: 130° C.
compound 5-1401: 191-193° C.
compound 5-1422: 180-182° C.

In addition, NMR data for compounds of the formula (I) according to the invention were generated. “NMRs” of the exemplary compounds were in each case measured as ¹H-NMR spectrum at 300 or 400 MHz (CDCl₃) (¹H nuclear magnetic resonance data). Characteristic chemical shifts δ (ppm) for some exemplary compounds are shown below:

NMR Compound 5-34 (CDCl₃, 400 MHz, δ in ppm):

2.42 (s, 3H); 3.53 (s, 2H); 6.69 (d, 1H); 6.92 (dd, 1H); 7.18 (d, 1H); 7.25 (m, 1H); 7.52 (d, 1H); 7.76 (td, 1H); 8.68 (dt, 1H).

NMR Compound 5-57 (CDCl₃, 400 MHz): 2.36 (s, 3H); 3.42 (s, 2H); 6.89 (m, 2H); 7.19 (m, 1H); 7.22 (d, 2H); 7.37 (d, 2H).

NMR Compound 5-99 (CDCl₃, 400 MHz): 2.39 (s, 3H); 3.45 (s, 2H); 7.23 (m, 3H); 7.33 (d, 1H); 7.42 (d, 2H).

NMR Compound 5-120 (CDCl₃, 400 MHz): 2.33 (s, 3H); 3.40 (s, 2H); 3.72 (s, 3H); 7.17 (s, 1H); 7.24 (d, 2H); 7.31 (s, 1H); 7.38 (d, 2H).

NMR Compound 5-165 (CDCl₃, 300 MHz, δ in ppm):

2.38 (s, 3H); 3.40 (s, 2H); 3.68 (s, 3H); 5.36 (s, 1H); 6.37 (t, 1H, J=71 Hz); 7.32 (m, 2H); 7.39 (m, 2H).

NMR Compound 5-477 ([D₆]-DMSO, 400 MHz): 2.28 (s, 3H); 3.37 (s, 2H); 7.32 (d, 2H); 7.53 (d, 2H).

NMR Compound 5-685 (CDCl₃, 400 MHz, δ in ppm):

2.13 (s, 3H); 2.32 (s, 3H); 2.41 (s, 3H); 3.46 (s, 2H); 7.47 (m, 1H); 7.71 (d, 1H); 7.83 (td, 1H); 8.65 (dt, 1H).

NMR Compound 5-708 (CDCl₃, 400 MHz): 2.32 (s, 3H); 3.38 (s, 2H); 6.44 (t, 1H); 7.33 (d, 2H); 7.42 (d, 2H).

NMR Compound 5-792 ([D₆]-DMSO, 400 MHz): 2.22 (s, 3H); 3.38 (s, 2H); 7.12 (d, 2H); 7.42 (d, 2H); 9.70 (s, 1H).

NMR Compound 5-897 (CDCl₃, 400 MHz): 2.34 (s, 3H); 3.45 (s, 2H); 3.66 (s, 3H); 3.71 (s, 3H); 7.08 (d, 1H); 7.13 (d, 2H); 7.26 (d, 2H); 7.47 (d, 1H).

NMR Compound 5-939 (CDCl₃, 400 MHz): 2.32 (s, 3H); 3.35 (s, 2H); 3.68 (s, 3H); 3.83 (s, 3H); 7.17 (s, 1H); 7.24 (d, 2H); 7.32 (s, 1H); 7.38 (d, 2H).

NMR Compound 5-984 (CDCl₃, 300 MHz, δ in ppm):

2.36 (s, 3H); 3.38 (s, 2H); 3.69 (2 s, 6H); 5.35 (s, 1H); 6.36 (t, 1H, J=71 Hz); 7.30 (m, 2H); 7.39 (m, 2H).

NMR Compound 5-1005 (CDCl₃, 300 MHz): 2.32 (s, 3H); 3.36 (s, 2H); 3.67 (s, 3H); 5.18 (s, 2H); 7.12 (m, 2H); 7.26 (m, 7H); 7.39 (m, 3H).

NMR Compound 5-1044 (CDCl₃, 400 MHz): 1.10 (t, 3H); 2.32 (s, 3H); 2.47 (s, 3H); 2.66 (s, 3H); 3.42 (s, 2H); 3.69 (s, 3H); 4.07 (q, 2H); 7.27 (m, 4H).

NMR Compound 5-1065 (CDCl₃, 400 MHz): 2.32 (s, 3H); 2.48 (s, 3H); 3.45 (s, 2H); 3.61 (s, 3H); 3.72 (s, 3H); 7.14 (d, 2H); 7.34 (d, 2H).

NMR Compound 5-1081 (CDCl₃, 400 MHz): 2.32 (s, 3H); 3.62 (s, 2H); 3.65 (s, 3H); 3.68 (s, 3H); 6.95 (s, 1H); 7.24 (s, 1H); 7.38 (d, 1H); 7.60 (dd, 1H); 8.56 (d, 1H).

NMR Compound 5-1102 (CDCl₃, 400 MHz): 2.34 (s, 3H); 3.43 (s, 3H); 3.65 (s, 2H); 3.71 (s, 3H); 6.94 (s, 1H); 7.20 (d, 1H); 7.43 (s, 1H); 7.61 (dd, 1H); 8.50 (d, 1H).

NMR Compound 5-1128 (CDCl₃, 400 MHz): 2.23 (s, 3H); 3.36 (s, 2H); 3.69 (s, 3H); 6.93 (s, 2H); 7.18 (d, 2H); 7.37 (d, 2H).

NMR Compound 5-1191 (CDCl₃, 400 MHz): 2.33 (s, 3H); 2.41 (s, 3H); 3.32 (s, 2H); 3.68 (s, 3H); 7.34 (d, 2H); 7.46 (d, 2H).

NMR Compound 5-1212 (CDCl₃, 400 MHz): 2.31 (s, 3H); 2.40 (s, 3H); 3.32 (s, 2H); 3.68 (s, 3H); 7.33 (d, 2H); 7.44 (d, 2H).

NMR Compound 5-1254 (CDCl₃, 400 MHz): 2.30 (s, 3H); 2.66 (s, 3H); 3.33 (s, 2H); 3.69 (s, 3H); 7.32 (d, 2H); 7.40 (d, 2H).

NMR Compound 5-1275 (CDCl₃, 400 MHz): 2.31 (s, 3H); 3.33 (s, 2H); 3.70 (s, 3H); 7.35 (d, 2H); 7.46 (d, 2H).

NMR Compound 5-1296 (CDCl₃, 400 MHz): 2.33 (s, 3H); 3.40 (s, 2H); 3.72 (s, 3H); 7.31 (d, 2H); 7.43 (d, 2H).

NMR Compound 5-1378 (CDCl₃, 400 MHz, δ in ppm):

1.24 (d, 6H); 2.35 (s, 3H); 3.01 (sept, 1H); 3.56 (s, 2H); 3.65 (s, 3H); 7.28 (m, 1H); 7.47 (d, 1H); 7.75 (td, 1H); 8.66 (dt, 1H).

NMR Compound 5-1380 (CDCl₃, 400 MHz): 1.32 (d, 6H); 2.34 (s, 3H); 3.06 (sept, 1H); 3.37 (s, 2H); 3.68 (s, 3H); 7.28 (d, 2H); 7.34 (d, 2H); 10.42 (broad s, 1H).

NMR Compound 5-1401 (CDCl₃, 400 MHz, δ in ppm):

0.95 (m, 4H); 1.87 (m, 1H); 2.33 (s, 3H); 3.56 (s, 2H); 3.64 (s, 3H); 7.28 (m, 1H); 7.40 (d, 1H); 7.72 (t, 1H); 8.64 (d, 1H).

NMR Compound 5-1485 (CDCl₃, 400 MHz): 2.21 (s, 3H); 2.32 (s, 3H); 3.32 (s, 2H); 3.68 (s, 3H); 6.58 (s, 1H); 7.32 (d, 2H); 7.39 (d, 2H).

NMR Compound 5-1504 (CDCl₃, 400 MHz, δ in ppm):

2.07 (s, 3H); 2.26 (s, 3H); 2.32 (s, 3H); 3.53 (s, 2H); 3.63 (s, 3H); 7.28 (m, 1H); 7.47 (d, 1H); 7.72 (td, 1H); 8.66 (dt, 1H).

NMR Compound 5-1506 (CDCl₃, 400 MHz): 2.12 (s, 3H); 2.24 (s, 3H); 2.32 (s, 3H); 3.33 (s, 2H); 3.67 (s, 3H); 7.30 (d, 2H); 7.38 (d, 2H).

NMR Compound 5-1548 (CDCl₃, 400 MHz): 2.29 (s, 3H); 3.33 (s, 2H); 3.69 (s, 3H); 7.34 (d, 2H); 7.41 (d, 2H).

NMR Compound 5-1569 (CDCl₃, 400 MHz): 2.29 (s, 3H); 3.31 (s, 2H); 3.68 (s, 3H); 7.31 (d, 2H); 7.43 (d, 2H).

NMR Compound 5-1590 (CDCl₃, 400 MHz): 2.33 (s, 3H); 3.48 (s, 2H); 3.69 (s, 3H); 6.97 (s, 1H); 7.23 (d, 2H); 7.35 (d, 2H); 7.64 (s, 1H).

NMR Compound 5-1632 (CDCl₃, 400 MHz): 2.29 (s, 3H); 2.31 (s, 3H); 3.28 (s, 2H); 3.67 (s, 3H); 6.29 (s, 1H); 7.32 (d, 2H); 7.49 (d, 2H).

NMR Compound 5-3431 (CDCl₃, 400 MHz): 2.34 (s, 3H); 3.53 (s, 2H); 3.66 (s, 3H); 7.32 (d, 1H); 7.45 (d, 1H); 7.85 (dd, 1H); 8.58 (d, 1H); 8.68 (d, 1H).

NMR Compound 5-3667 (CDCl₃, 400 MHz): 2.34 (s, 3H); 3.35 (s, 2H); 3.69 (s, 3H); 7.21 (d, 1H); 7.26 (d, 2H); 7.37 (d, 2H); 8.54 (d, 1H).

NMR Compound 5-4375 (CDCl₃, 400 MHz): 2.42 (s, 3H); 3.46 (s, 2H); 7.36 (d, 1H); 7.56 (d, 1H); 7.93 (dd, 1H); 8.68 (d, 1H); 8.75 (d, 1H).

NMR Compound 5-4611 (CDCl₃, 400 MHz): 2.36 (s, 3H); 3.40 (s, 2H); 7.21 (d, 1H); 7.26 (d, 2H); 7.39 (d, 2H); 8.55 (d, 1H).

### (B) FORMULATION EXAMPLES

a) A dust is obtained by mixing 10 parts by weight of a compound of the formula (I) and 90 parts by weight of talc as inert substance and comminuting the mixture in a hammer mill.
b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurate as wetting agent and dispersant, and grinding the mixture in a pinned-disk mill.
c) A readily water-dispersible dispersion concentrate is obtained by mixing 20 parts by weight of a compound of the formula (I) with 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to above 277° C.) and grinding the mixture in a ball mill to a fineness of below 5 microns.
d) An emulsifiable concentrate is obtained from 15 parts by weight of a compound of the formula (I), 75 parts by weight of cyclohexanone as solvent and 10 parts by weight of oxethylated nonylphenol as emulsifier.
e) Water-dispersible granules are obtained by mixing
   75 parts by weight of a compound of the formula (I)
      10″ of calcium lignosulfonate,
      5″ of sodium lauryl sulfate,
      3″ of polyvinyl alcohol and
      7″ of kaolin,
   grinding the mixture in a pinned-disk mill, and granulating the powder in a fluidized bed by spraying on water as granulating liquid.
f) Water-dispersible granules are also obtained by homogenizing and precomminuting, in a colloid mill,
   25 parts by weight of a compound of the formula (I),
   5″ of sodium 2,2′-dinaphthylmethane-6,6′-disulfonate,
   2″ of sodium oleoylmethyltaurate,
   1 part by weight of polyvinyl alcohol,
   17 parts by weight of calcium carbonate and
   50″ of water,
   subsequently grinding the mixture in a bead mill and atomizing and drying the resulting suspension in a spray tower by means of a single-substance nozzle.

### (C) BIOLOGICAL EXAMPLES

### 1. Pre-Emergence Herbicidal Effect and Crop Plant Compatibility

Seeds of monocotyledonous and dicotyledonous weed plants and crop plants are placed in wood-fiber pots in sandy loam and covered with soil. The compounds (I) according to the invention, formulated in the form of wettable powders (WP), are then applied as aqueous suspension or emulsion at a water application rate of 600 I/ha (converted) with the addition of 0.2% of wetting agent to the surface of the covering soil.

After the treatment, the pots are placed in a greenhouse and kept under good growth conditions for the test plants. After about 3 weeks, the effect of the preparations is scored visually in comparison with untreated controls (herbicidal effect in percent (%): 100% activity=the plants have died, 0% activity=like control plants).

Compounds (I) according to the invention, such as, for example, the compounds of Tables 4 and 5, applied at an application rate of 1 kg or less of active substance per hectare, have good herbicidal pre-emergence activity against a number of harmful plants. Furthermore, compounds of Tables 4 and 5, such as, for example, the compounds Nos. 5-99, 5-120, 5-1081, 5-1254, 5-1422, 5-1506 and 5-1548, applied by the pre-emergence method at an application rate of 0.32 kg or less of active substance per hectare, have very good herbicidal activity (90% and more herbicidal activity) against harmful plants such as Viola tricolor. Furthermore, the compounds Nos. 5-99, 5-120 and 5-1548, for example, applied by the pre-emergence method at an application rate of 0.32 kg or less of active substance per hectare, have good herbicidal activity (80% and more) against harmful plants such as Veronica persica. Furthermore, the compounds Nos. 5-120 and 5-1548, for example, applied by the pre-emergence method at an application rate of 0.32 kg or less of active substance per hectare, have very good herbicidal activity (90% and more) against harmful plants such as Amaranthus retroflexus. Furthermore, the compounds Nos. 5-1254 and 5-1506, for example, applied by the pre-emergence method at an application rate of 0.32 kg or less of active substance per hectare, have good herbicidal activity (80% and more) against harmful plants such as Lolium multiflorum. Furthermore, the compound No. 5-1422, for example, applied by the pre-emergence method at an application rate of 0.32 kg or less of active substance per hectare, has very good herbicidal activity (90% and more) against harmful plants such as Polygonum convulvus. However, in general, these and other active compounds of Tables 4 and have a substantially broader activity spectrum.

At the same time, compounds according to the invention applied by the pre-emergence method do not damage dicotyledonous crops such as oilseed rape even at high active compound dosages and additionally also spare gramineous crops such as wheat, corn and rice. Some of the compounds according to the invention have high selectivity and are therefore suitable for controlling unwanted vegetation in agricultural crops by the pre-emergence method.

### 2. Post-Emergence Herbicidal Effect and Crop Plant Compatibility

Seeds of monocotyledonous and dicotyledonous weed and crop plants are placed in sandy loam in wood-fiber pots, covered with soil and cultivated in a greenhouse under good growth conditions. 2 to 3 weeks after sowing, the test plants are treated at the one-leaf stage. The compounds (I) according to the invention, formulated in the form of wettable powders (WP), are then applied by spraying as aqueous suspension or emulsion at a water application rate of 600 I/ha (converted) with the addition of 0.2% of wetting agent to the green parts of the plants. After the test plants have been kept in the greenhouse under optimum growth conditions for about 3 weeks, the activity of the preparations is rated visually in comparison to untreated controls (herbicidal activity in percent (%): 100% activity=the plants have died, 0% activity=like control plants).

The results show that compounds (I) according to the invention such as, for example, the compounds from Tables 4 and 5, applied at an application rate of 1 kg and less of active substance per hectare, have good herbicidal post-emergence activity against a broad spectrum of weed-grasses and broad-leaved weeds. Furthermore, individual compounds from Tables 4 and 5, such as, for example, the compound No. 5-1107, applied at 0.32 kg of active substance per hectare, have very good herbicidal activity (80% and more) against harmful plants such as Avena fatua, Echinochloa crus galli, Alopecurus myosuroides and Abutilon theophrasti. The compound 5-1102, applied at 0.32 kg of active substance per hectare, showed very good activity against Abutilon theophrasti (80%) and Avena fatua (90%). However, in general, these and other active compounds of Tables 4 and 5 have a substantially broader activity spectrum.

What is claimed is:

1. A compound of the formula (I) or a salt thereof 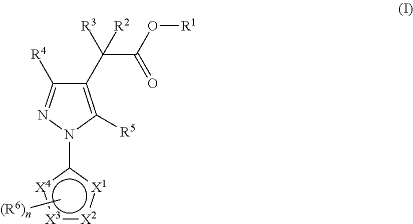
in which
each of the divalent groups X¹, X², X³ and X⁴ is a group of the formula CH, N, NH, O or S, with the proviso that the ring attached at the 1-N atom of the pyrazole ring is a heteroaromatic five-membered ring which is unsubstituted or substituted by radicals R⁶, where in the case of a substitution a hydrogen atom at a group CH or NH is replaced by a radical R⁶,
except for optionally substituted five-membered heteroaromatic radicals having two heteroatoms as ring atoms in which one of the heteroatoms in the ring is a nitrogen atom and the other is a sulfur atom and the nitrogen atom in the ring is located in the 1,3-position to the ring carbon atom which is attached at the 1-N atom of the pyrazole radical,
R¹ is hydrogen or a hydrolyzable radical,
R² is hydrogen, halogen or (C₁-C₆)-alkyl which is unsubstituted or substituted by one or more radicals selected from the group consisting of halogen, (C₁-C₄)-alkoxy, (C₁-C₄)-alkylthio and (C₁-C₄)-haloalkoxy,
R³ is hydrogen, halogen or (C₁-C₆)-alkyl which is unsubstituted or substituted by one or more radicals selected from the group consisting of halogen, (C₁-C₄)-alkoxy, (C₁-C₄)-alkylthio and (C₁-C₄)-haloalkoxy, or
R² and R³ together with the carbon atom to which they are attached are a carbocyclic saturated or partially unsaturated ring having 3 to 6 carbon atoms which is unsubstituted or substituted by one or more radicals selected from the group consisting of halogen and (C₁-C₄)-alkyl, and
R⁴ is hydrogen, halogen, cyano, (C₁-C₆)-alkyl, (C₂-C₆)-alkenyl or (C₂-C₆)-alkynyl,
where each of the three lastmentioned radicals is unsubstituted or substituted by one or more radicals selected from the group consisting of halogen, cyano, hydroxyl, (C₁-C₄)-alkoxy, (C₁-C₄)-haloalkoxy, (C₁-C₄)-alkoxy-(C₁-C₄)-alkoxy, (C₁-C₄)-alkylthio and optionally halogen-, cyano-, (C₁-C₄)-alkyl- or (C₁-C₄)-haloalkyl-substituted (C₃-C₉)-cycloalkyl, or
is (C₃-C₉)-cycloalkyl, (C₅-C₉)-cycloalkenyl or (C₅-C₉)-cycloalkynyl, where each of the 3 lastmentioned radicals is unsubstituted or substituted by one or more radicals selected from the group consisting of halogen, (C₁-C₄)-alkyl, (C₁-C₄)-haloalkyl, (C₂-C₄)-alkenyl, (C₂-C₄)-alkynyl, (C₁-C₄)-alkoxy, (C₁-C₄)-haloalkoxy and (C₁-C₄)-alkylthio, or
is phenyl which is unsubstituted or substituted by one or more radicals selected from the group consisting of halogen, cyano, nitro, carboxyl, (C₁-C₄)-alkyl, (C₁-C₄)-haloalkyl, (C₂-C₄)-alkenyl, (C₂-C₄)-alkynyl, (C₁-C₄)-alkoxy, (C₁-C₄)-haloalkoxy, (C₁-C₄)-alkoxy-(C₁-C₄)-alkoxy, (C₁-C₄)-alkylthio, (C₁-C₄)-alkanoyl, (C₁-C₄)-haloalkanoyl, [(C₁-C₄)-alkoxy]carbonyl and [(C₁-C₄)-haloalkoxy]carbonyl, or
is (C₁-C₆)-alkanoyl which is unsubstituted or substituted by one or more radicals selected from the group consisting of halogen, cyano, (C₁-C₄)-alkoxy, (C₁-C₄)-haloalkoxy, (C₁-C₄)-alkoxy-(C₁-C₄)-alkoxy, (C₁-C₄)-alkylthio and optionally halogen-, cyano-, (C₁-C₄)-alkyl- or (C₁-C₄)-haloalkyl-substituted (C₃-C₆)-cycloalkyl, or
is [(C₁-C₄)-alkoxy]carbonyl which is unsubstituted or substituted by one or more radicals selected from the group consisting of halogen, cyano, (C₁-C₄)-alkoxy, (C₁-C₄)-haloalkoxy, (C₁-C₄)-alkoxy-(C₁-C₄)-alkoxy, (C₁-C₄)-alkylthio and optionally halogen-, cyano-, (C₁-C₄)-alkyl- or (C₁-C₄)-haloalkyl-substituted (C₃-C₆)-cycloalkyl, or
is [(C₃-C₉)-cycloalkoxy]carbonyl which is unsubstituted or substituted by one or more radicals selected from the group consisting of halogen, (C₁-C₄)-alkyl, (C₁-C₄)-haloalkyl, (C₂-C₄)-alkenyl, (C₂-C₄)-alkynyl, (C₁-C₄)-alkoxy, (C₁-C₄)-haloalkoxy and (C₁-C₄)-alkylthio,
R⁵ is an aryl radical which is unsubstituted or substituted and, including substituents, has 6 to 30 carbon atoms, or
is a heteroaromatic radical having 1 to 4 ring heteroatoms selected from the group consisting of N, O and S which is unsubstituted or substituted and, including substituents, has 1 to 30 carbon atoms, and
(R⁶)ₙ are n substituents R⁶, where R⁶, in the case that n=1, or each of the substituents R⁶ independently of the others, in the case that n is greater than 1, is a radical halogen, hydroxyl, amino, nitro, carboxyl, cyano, carbamoyl, (C₁-C₆)-alkyl, (C₁-C₆)-haloalkyl, (C₁-C₄)-alkoxy-(C₁-C₄)-alkyl, (C₁-C₄)-alkylthio-(C₁-C₄)-alkyl, mono- or di-[(C₁-C₄)-alkyl]aminoalkyl, hydroxy-(C₁-C₄)-alkyl, (C₁-C₆)-alkyl which is substituted
by optionally halogen-, cyano-, (C₁-C₄)-alkyl- or (C₁-C₄)-haloalkyl-substituted (C₃-C₆)-cycloalkyl,
by optionally halogen-, cyano-, (C₁-C₄)-alkyl-, (C₁-C₄)-haloalkyl- or (C₁-C₄)-alkoxy-substituted phenyl or
by optionally halogen-, cyano-, (C₁-C₄)-alkyl-, (C₁-C₄)-haloalkyl- or (C₁-C₄)-alkoxy-substituted heterocyclyl,
or (C₂-C₆)-alkenyl, (C₂-C₆)-haloalkenyl, (C₂-C₆)-alkynyl, (C₂-C₆)-haloalkynyl, (C₁-C₆)-alkoxy, (C₁-C₆)-haloalkoxy, (C₁-C₄)-alkoxy-(C₁-C₄)-alkoxy, (C₁-C₆)-alkylthio, (C₁-C₆)-haloalkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- or di-[(C₁-C₄)-alkyl]aminocarbonyl, mono- or di-[(C₁-C₆)-acyl]amino, mono- or di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₃-C₉)-cycloalkyl, (C₅-C₉)-cycloalkenyl,
where each of the two lastmentioned radicals is unsubstituted or substituted by one or more radicals selected from the group consisting of halogen, (C₁-C₄)-alkyl and (C₁-C₄)-haloalkyl,
or optionally halogen-, cyano-, (C₁-C₄)-alkyl-, (C₁-C₄)-haloalkyl- or (C₁-C₄)-alkoxy-substituted heterocyclyl, and
n is 0, 1, 2 or 3.

2. The compound or the salt thereof as claimed in claim 1 wherein
R¹ is hydrogen or an optionally substituted hydrocarbon radical or an optionally substituted heterocyclyl radical, where each of the two lastmentioned carbon-containing radicals including substituents has 1 to 30 carbon atoms, or a radical of the formula SiR^{a}R^{b}R^{c}, —NR^{a}R^{b} or —N═CR^{c}R^{d}, where in the 3 lastmentioned formulae each of the radicals R^{a}, R^{b}, R^{c} and R^{d} independently of the other is hydrogen or an optionally substituted hydrocarbon radical or R^{a} and R^{b} together with the nitrogen atom are a 3- to 9-membered heterocycle which in addition to the nitrogen atom may contain one or two further ring heteroatoms selected from the group consisting of N, O and S and which is unsubstituted or substituted, or R^{c} and R^{d} together with the carbon atom are a 3- to 9-membered carbocyclic radical or a heterocyclic radical which may contain 1 to 3 ring heteroatoms selected from the group consisting of N, O and S, where the carbocyclic or heterocyclic radical is unsubstituted or substituted,
where each of the radicals R^{a}, R^{b}, R^{c} and R^{d} including substituents has up to 30 carbon atoms.

3. The compound or the salt thereof as claimed in claim 1 wherein
R¹ is hydrogen, alkyl, alkenyl, alkynyl, cycloalkyl, cycloalkenyl, cycloalkynyl or aryl, where each of the 7 lastmentioned radicals is unsubstituted or substituted and, including substituents, has up to 30 carbon atoms, or a heterocyclyl radical having 3 to 9 ring atoms which contains 1 to 4 heteroatoms selected from the group consisting of N, O and S, which is unsubstituted or substituted and which, including substituents, has 1 to 30 carbon atoms.

4. The compound or the salt thereof as claimed in claim 1 wherein
R¹ is H, (C₁-C₁₈)-alkyl, (C₂-C₁₈)-alkenyl, (C₂-C₁₈)-alkynyl, (C₃-C₉)-cycloalkyl, (C₅-C₉)-cycloalkenyl, (C₅-C₉)-cycloalkynyl or phenyl,
where each of the 7 lastmentioned radicals is unsubstituted or substituted by one or more radicals selected from the group consisting of halogen, cyano, thio, nitro, hydroxyl, carboxyl, (C₁-C₈)-alkyl, (C₁-C₈)-haloalkyl, (C₁-C₄)-alkoxy-(C₁-C₄)-alkyl, (C₂-C₈)-alkenyl, (C₂-C₈)-haloalkenyl, (C₂-C₈)-alkynyl, (C₂-C₈)-haloalkynyl, the 7 lastmentioned radicals only in the case of cyclic base radicals, (C₁-C₈)-alkoxy, (C₂-C₈)-alkenyloxy, (C₂-C₈)-alkynyloxy, (C₁-C₈)-haloalkoxy, (C₁-C₄)-alkoxy-(C₁-C₄)-alkoxy, (C₁-C₈)-alkylthio, (C₂-C₈)-alkenylthio, (C₂-C₈)-alkynylthio, radicals of the formulae —NR*R**, —CO—NR*R** and —O—CO—NR*R**,
where each of the radicals R* and R** in the 3 lastmentioned formulae independently of the others is H, (C₁-C₈)-alkyl, (C₂-C₈)-alkenyl, (C₂-C₈)-alkynyl, benzyl, substituted benzyl, phenyl or substituted phenyl, or together with the nitrogen atom is a 3- to 8-membered heterocycle which, in addition to the nitrogen atom, may contain one or two further ring heteroatoms selected from the group consisting of N, O and S and which is unsubstituted or substituted by one or more radicals selected from the group consisting of (C₁-C₄)-alkyl and (C₁-C₄)-haloalkyl,
and [(C₁-C₈)-alkoxy]carbonyl, [(C₁-C₈)-alkoxy]thiocarbonyl, [(C₂-C₈)-alkenyloxy]carbonyl, [(C₂-C₈)-alkynyloxy]carbonyl, [(C₁-C₈)-alkylthio]carbonyl, [(C₂-C₈)-alkenylthio]carbonyl, [(C₂-C₈)-alkynylthio]carbonyl, (C₁-C₈)-alkanoyl, [(C₂-C₈)-alkenyl]carbonyl, [(C₂-C₈)-alkynyl]carbonyl, (C₁-C₄)-alkylimino, (C₁-C₄)-alkoxyimino, [(C₁-C₈)-alkyl]carbonylamino, [(C₂-C₈)-alkenyl]carbonylamino, [(C₂-C₈)-alkynyl]carbonylamino, [(C₁-C₈)-alkoxy]carbonylamino, [(C₂-C₈)-alkenyloxy]carbonylamino, [(C₂-C₈)-alkynyloxy]carbonylamino, [(C₁-C₈)-alkylamino]carbonylamino, [(C₁-C₆)-alkyl]carbonyloxy, [(C₂-C₆)-alkenyl]carbonyloxy, [(C₂-C₆)-alkynyl]carbonyloxy, [(C₁-C₈)-alkoxy]carbonyloxy, [(C₂-C₈)-alkenyloxy]carbonyloxy, [(C₂-C₈)-alkynyloxy]carbonyloxy, (C₁-C₈)-alkylsulfinyl and (C₁-C₈)-alkylsulfonyl,
where each of the 27 lastmentioned radicals is unsubstituted or substituted by one or more radicals selected from the group consisting of halogen, NO₂, (C₁-C₄)-alkoxy and optionally substituted phenyl,
and phenyl, phenyl-(C₁-C₆)-alkoxy, phenyl-[(C₁-C₆)-alkoxy]carbonyl, phenoxy, phenoxy-(C₁-C₆)-alkoxy, phenoxy-[(C₁-C₆)-alkoxy]carbonyl, phenoxycarbonyl, phenylcarbonyloxy, phenoxycarbonyloxy, phenylcarbonylamino, phenyl-[(C₁-C₆)-alkyl]carbonylamino, phenyl-[(C₁-C₆)-alkyl]carbonyloxy, phenyl-[(C₁-C₆)-alkoxy]carbonyloxy, (C₃-C₇)-cycloalkyl, (C₃-C₇)-cycloalkoxy, (C₃-C₆)-cycloalkyl-(C₁-C₆)-alkoxy, (C₃-C₆)-cycloalkyl-[(C₁-C₆)-alkoxy]carbonyl, (C₃-C₆)-cycloalkoxy-(C₁-C₆)-alkoxy, (C₃-C₆)-cycloalkoxy-[(C₁-C₆)-alkoxy]carbonyl, (C₃-C₆)-cycloalkoxycarbonyl, (C₃-C₆)-cycloalkylcarbonyloxy, (C₃-C₆)-cycloalkoxycarbonyloxy, (C₃-C₆)-cycloalkyl-[(C₁-C₆)-alkoxy]carbonyloxy, (C₃-C₆)-cycloalkylcarbonylamino, (C₃-C₆)-cycloalkyl-[(C₁-C₆)-alkyl]carbonylamino and (C₃-C₆)-cycloalkyl-[(C₁-C₆)-alkyl]carbonyloxy,
where each of the 26 lastmentioned radicals is optionally also fused, preferably benzo-fused, with a carbocyclic or heterocyclic ring, preferably a carbocyclic ring having 3 to 6 carbon atoms or a heterocyclic ring having 5 or 6 ring atoms and 1 to 3 ring heteroatoms selected from the group consisting of N, O and S, and is unsubstituted in the ring or in the polycyclic system or is substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
and radicals of the formulae —SiR′₃, —O—SiR′₃, (R′)₃Si—(C₁-C₆)-alkoxy, —CO—O—NR′₂, —O—N═CR′₂, —N═CR′₂, —O—NR′₂, —CH(OR′)₂ and —O—(CH₂)ₘ—CH(OR′)₂,
in which each of the radicals R′ independently of the others is H, (C₁-C₄)-alkyl or phenyl which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro or substituted in two adjacent positions by a (C₂-C₆)-alkylene bridge, and m is an integer of from 0 to 6,
and radicals of the formula R″O—CHR′″CH(OR″)—(C₁-C₆)-alkoxy,
in which each of the radicals R″ independently of the others is H or (C₁-C₄)-alkyl or the radicals R″ together are a (C₁-C₆)-alkylene group and R′″ is H or (C₁-C₄)-alkyl,
and also radicals of the formula Het¹, where Het¹ is in each case independently of the others a saturated, partially unsaturated or heteroaromatic heterocyclyl radical having 3 to 9 ring atoms, where the heterocyclic radical in question contains 1 to 4 heteroatoms, selected from the group consisting of N, O and S and is optionally also fused, with a carbocyclic or heterocyclic ring and is unsubstituted in the ring or in the polycyclic system or is substituted by one or more radicals selected from the group consisting of halogen, cyano, thio, nitro, hydroxyl, carboxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, (C₃-C₆)-cycloalkyl, (C₃-C₆)-cycloalkoxy, [(C₁-C₈)-alkoxy]carbonyl, [(C₁-C₆)-haloalkoxy]carbonyl and oxo,
or
R¹ is a polycyclic radical based on (C₃-C₉)-cycloalkyl, (C₅-C₉)-cycloalkenyl, (C₅-C₉)-cycloalkynyl or phenyl, where the basic ring is fused with a carbocyclic or heterocyclic ring and where the basic ring or the polycyclic system is unsubstituted or substituted by one or more radicals selected from the group consisting of halogen, cyano, thio, nitro, hydroxyl, carboxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, (C₃-C₆)-cycloalkyl, (C₃-C₆)-cycloalkoxy, [(C₁-C₈)-alkoxy]carbonyl, [(C₁-C₆)-haloalkoxy]carbonyl and oxo,
or
R¹ is a saturated, partially unsaturated or heteroaromatic heterocyclyl radical having 3 to 9 ring atoms which contains 1 to 4 heteroatoms selected from the group consisting of N, O and S, optionally also fused with a carbocyclic or heterocyclic ring, and which is unsubstituted in the ring or in the polycyclic system or is substituted by one or more radicals selected from the group consisting of halogen, cyano, thio, nitro, hydroxyl, carboxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, (C₃-C₆)-cycloalkyl, (C₃-C₆)-cycloalkoxy, [(C₁-C₈)-alkoxy]carbonyl, [(C₁-C₆)-haloalkoxy]carbonyl and oxo.

5. The compound or the salt thereof as claimed in claim 1 wherein
R¹ is H, (C₁-C₁₂)-alkyl, (C₂-C₁₂)-alkenyl, (C₂-C₁₂)-alkynyl, (C₃-C₆)-cycloalkyl, (C₅-C₆)-cycloalkenyl, (C₅-C₆)-cycloalkynyl or phenyl,
where each of the 7 lastmentioned radicals is unsubstituted or substituted by one or more radicals selected from the group consisting of halogen, cyano, thio, nitro, hydroxyl, (C₁-C₆)-alkyl, (C₁-C₆)-haloalkyl, (C₁-C₄)-alkoxy-(C₁-C₄)-alkyl, (C₂-C₆)-alkenyl, (C₂-C₆)-haloalkenyl, (C₂-C₆)-alkynyl, (C₂-C₆)-haloalkynyl, the 7 lastmentioned radicals only in the case of cyclic base radicals, (C₁-C₆)-alkoxy, (C₂-C₆)-alkenyloxy, (C₂-C₆)-alkynyloxy, (C₁-C₆)-haloalkoxy, (C₁-C₄)-alkoxy-(C₁-C₄)-alkoxy, (C₁-C₆)-alkylthio, (C₂-C₆)-alkenylthio, (C₂-C₆)-alkynylthio, radicals of the formulae —NR*R**, —CO—NR*R** and —O—CO—NR*R**,
where each of the radicals R* and R** in the 3 lastmentioned formulae independently of the others is H, (C₁-C₄)-alkyl, (C₂-C₄)-alkenyl, (C₂-C₄)-alkynyl, benzyl, phenyl which is unsubstituted or substituted by one or more radicals selected from the group consisting of halogen, (C₁-C₄)-alkyl, (C₁-C₄)-haloalkyl and (C₁-C₄)-alkoxy, or together with the nitrogen atom is a piperidine, piperazine, pyrrolidine, pyrazolidine, piperazolidine or morpholine radical which is unsubstituted or substituted by one or more radicals selected from the group consisting of (C₁-C₄)-alkyl and (C₁-C₄)-haloalkyl,
and [(C₁-C₄)-alkoxy]carbonyl, [(C₁-C₄)-alkyl]carbonylamino, [(C₁-C₄)-alkoxy]carbonylamino, [(C₁-C₄-alkylamino]carbonylamino, [(C₁-C₄)-alkyl]carbonyloxy, [(C₁-C₄)-alkoxy]carbonyloxy and (C₁-C₄)-alkylsulfonyl,
where each of the 7 lastmentioned radicals is unsubstituted or substituted by one or more radicals selected from the group consisting of halogen, NO₂, (C₁-C₄)-alkoxy and phenyl, which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
and phenyl, phenyl-(C₁-C₄)-alkoxy, phenyl-[(C₁-C₄)-alkoxy]carbonyl, phenoxy, phenoxy-(C₁-C₄)-alkoxy, phenoxy-[(C₁-C₄)-alkoxy]carbonyl, phenoxycarbonyl, phenylcarbonyloxy, phenylcarbonylamino, phenyl-[(C₁-C₄)-alkyl]carbonylamino, phenyl-[(C₁-C₄)-alkyl]carbonyloxy, (C₃-C₆)-cycloalkyl and (C₃-C₆)-cycloalkoxy,
where each of the 13 lastmentioned radicals is unsubstituted in the ring or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro,
and radicals of the formulae —CO—O—NR′₂, —O—N═CR′₂, —N═CR′₂, —O—NR′₂, —CH(OR′)₂ and —O—(CH₂)ₘ—CH(OR′)₂,
in which each of the radicals R′ independently of the others is H, (C₁-C₄)-alkyl or phenyl which is unsubstituted or substituted by one or more radicals selected from the group consisting of halogen, (C₁-C₄)-alkyl, (C₁-C₄)-alkoxy, (C₁-C₄)-haloalkyl, (C₁-C₄)-haloalkoxy and nitro or is substituted in two adjacent positions by a (C₂-C₆)-alkylene bridge, and m is an integer of from 0 to 6,
and radicals of the formula R″O—CHR′″CH(OR″)—(C₁-C₆)-alkoxy,
in which each of the radicals R″ independently of the others is H or (C₁-C₄)-alkyl or the radicals R″ together are a (C₁-C₄)-alkylene group and R′″ is H or (C₁-C₂)-alkyl.

6. The compound or the salt thereof as claimed in claim 1 wherein
R² is hydrogen, halogen or (C₁-C₄)-alkyl which is unsubstituted or substituted by one or more radicals selected from the group consisting of halogen and
R³ is hydrogen, halogen or (C₁-C₄)-alkyl which is unsubstituted or substituted by one or more radicals selected from the group consisting of halogen or
R² and R³ together with the carbon atom to which they are attached are (C₃-C₆)-cycloalkyl or (C₅-C₆)-cycloalkenyl, where each of the 2 lastmentioned radicals is unsubstituted or substituted by one or more radicals selected from the group consisting of halogen and (C₁-C₄)-alkyl.

7. The compound or the salt thereof as claimed in claim 2 wherein
R² is hydrogen, halogen or (C₁-C₄)-alkyl which is unsubstituted or substituted by one or more radicals selected from the group consisting of halogen and
R³ is hydrogen, halogen or (C₁-C₄)-alkyl which is unsubstituted or substituted by one or more radicals selected from the group consisting of halogen or
R² and R³ together with the carbon atom to which they are attached are (C₃-C₆)-cycloalkyl or (C₅-C₆)-cycloalkenyl, where each of the 2 lastmentioned radicals is unsubstituted or substituted by one or more radicals selected from the group consisting of halogen and (C₁-C₄)-alkyl.

8. The compound or the salt thereof as claimed in claim 3 wherein
R² is hydrogen, halogen or (C₁-C₄)-alkyl which is unsubstituted or substituted by one or more radicals selected from the group consisting of halogen and
R³ is hydrogen, halogen or (C₁-C₄)-alkyl which is unsubstituted or substituted by one or more radicals selected from the group consisting of halogen or
R² and R³ together with the carbon atom to which they are attached are (C₃-C₆)-cycloalkyl or (C₅-C₆)-cycloalkenyl, where each of the 2 lastmentioned radicals is unsubstituted or substituted by one or more radicals selected from the group consisting of halogen and (C₁-C₄)-alkyl.

9. The compound or the salt thereof as claimed in claim 4 wherein
R² is hydrogen, halogen or (C₁-C₄)-alkyl which is unsubstituted or substituted by one or more radicals selected from the group consisting of halogen and
R³ is hydrogen, halogen or (C₁-C₄)-alkyl which is unsubstituted or substituted by one or more radicals selected from the group consisting of halogen or
R² and R³ together with the carbon atom to which they are attached are (C₃-C₆)-cycloalkyl or (C₅-C₆)-cycloalkenyl, where each of the 2 lastmentioned radicals is unsubstituted or substituted by one or more radicals selected from the group consisting of halogen and (C₁-C₄)-alkyl.

10. The compound or the salt thereof as claimed in claim 5 wherein
R² is hydrogen, halogen or (C₁-C₄)-alkyl which is unsubstituted or substituted by one or more radicals selected from the group consisting of halogen and
R³ is hydrogen, halogen or (C₁-C₄)-alkyl which is unsubstituted or substituted by one or more radicals selected from the group consisting of halogen or
R² and R³ together with the carbon atom to which they are attached are (C₃-C₆)-cycloalkyl or (C₅-C₆)-cycloalkenyl, where each of the 2 lastmentioned radicals is unsubstituted or substituted by one or more radicals selected from the group consisting of halogen and (C₁-C₄)-alkyl.

11. The compound or the salt thereof as claimed in claim 1 wherein
R⁴ is hydrogen, halogen, cyano, (C₁-C₄)-alkyl, (C₂-C₄)-alkenyl or (C₂-C₄)-alkynyl, where each of the three lastmentioned radicals is unsubstituted or substituted by one or more radicals selected from the group consisting of halogen and hydroxyl, or
(C₃-C₆)-cycloalkyl which is unsubstituted or substituted by one or more radicals selected from the group consisting of halogen and (C₁-C₄)-alkyl, or
phenyl which is unsubstituted or substituted by one or more radicals selected from the group consisting of halogen, nitro, (C₁-C₄)-alkyl, (C₁-C₄)-haloalkyl, (C₁-C₄)-alkoxy, (C₁-C₄)-haloalkoxy, (C₁-C₄)-alkoxy-(C₁-C₄)-alkoxy, (C₁-C₄)-alkylthio, [(C₁-C₄)-alkoxy]carbonyl and [(C₁-C₄)-haloalkoxy]carbonyl, or
(C₁-C₄)-alkanoyl which is unsubstituted or substituted by one or more radicals selected from the group consisting of halogen, cyano, (C₁-C₄)-alkoxy, (C₁-C₄)-haloalkoxy and (C₁-C₂)-alkoxy-(C₁-C₂)-alkoxy, or
[(C₁-C₄)-alkoxy]carbonyl which is unsubstituted or substituted by one or more radicals selected from the group consisting of halogen, or [(C₃-C₆)-cycloalkoxy]carbonyl which is unsubstituted or substituted by one or more radicals selected from the group consisting of halogen and (C₁-C₄)-alkyl.

12. The compound or the salt thereof as claimed in claim 1 wherein
R⁵ is a phenyl radical or a 5- or 6-membered heteroaromatic radical having 1 to 3 ring heteroatoms selected from the group consisting of N, O and S, where the phenyl radical or the heterocyclic radical is unsubstituted or substituted by one or more radicals selected from the group consisting of the radicals
(a) halogen, hydroxyl, amino, nitro, carboxyl, cyano and carbamoyl,
(b) (C₁-C₆)-alkyl, (C₂-C₆)-alkenyl, (C₂-C₆)-alkynyl, (C₁-C₆)-alkoxy, (C₁-C₆)-alkenyloxy and (C₁-C₆)-alkynyloxy, where each of the 6 lastmentioned radicals is unsubstituted or substituted by one or more radicals selected from the group consisting of halogen, (C₁-C₄)-alkoxy, (C₁-C₆)-haloalkoxy, (C₁-C₄)-alkylthio, mono- and di-[(C₁-C₄)-alkyl]amino, hydroxyl, carboxyl, [(C₁-C₄)-alkoxy]carbonyl, [(C₁-C₄)-haloalkoxy]carbonyl, mono- and di-[(C₁-C₄)-alkyl]aminocarbonyl and cyano,
(c) (C₁-C₆)-alkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- and di-[(C₁-C₄)-alkyl]aminocarbonyl, mono- and di-[(C₁-C₆)acyl]amino, mono- and di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₁-C₆)-alkylsulfinyloxy, (C₁-C₆)-haloalkylsulfinyloxy, (C₁-C₆)-alkylsulfonyloxy, (C₁-C₆)-haloalkylsulfonyloxy, (C₁-C₆)-alkylsulfato, (C₁-C₆)-haloalkylsulfato and
(d) (C₃-C₆)-cycloalkyl, (C₃-C₆)-cycloalkyloxy, phenyl and phenoxy,
where each of the 4 lastmentioned radicals is unsubstituted or substituted by one or more radicals selected from the group consisting of halogen, (C₁-C₄)-alkyl, (C₁-C₄)-haloalkyl, (C₁-C₄)-alkoxy, (C₁-C₆)-haloalkoxy and (C₁-C₄)-alkylthio,
and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen, (C₁-C₄)-alkyl, (C₁-C₄)-haloalkyl, (C₁-C₄)-alkoxy, (C₁-C₆)-haloalkoxy and (C₁-C₄)-alkylthio.

13. The compound or the salt thereof as claimed in claim 1 wherein
R⁵ is phenyl
which is unsubstituted or substituted by one or more radicals selected from the group consisting of halogen, hydroxyl, amino, nitro, carboxyl, cyano, carbamoyl, (C₁-C₆)-alkyl, (C₂-C₄)-alkenyl, (C₂-C₄)-alkynyl, (C₁-C₆)-haloalkyl, (C₁-C₄)-alkoxy-(C₁-C₄)-alkyl, (C₁-C₄)-alkylthio-(C₁-C₄)-alkyl, mono- and di-[(C₁-C₄)-alkyl]amino-(C₁-C₄)-alkyl, hydroxy-(C₁-C₄)-alkyl, carboxy-(C₁-C₄)-alkyl, cyano-(C₁-C₄)-alkyl, (C₁-C₆)-alkoxy, which may optionally also be halogenated[=(C₁-C₆)-haloalkoxy)], (C₁-C₄)-alkoxy-(C₁-C₄)-alkoxy, (C₁-C₆)-alkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- and di-[(C₁-C₄)-alkyl]aminocarbonyl, mono- and di-[(C₁-C₆)acyl]amino, mono- and di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₁-C₄)-alkylsulfonyloxy, (C₃-C₆)-cycloalkyl, (C₃-C₆)-cycloalkyloxy, phenyl and phenoxy,
where each of the 4 lastmentioned radicals is unsubstituted or substituted by one or more radicals selected from the group consisting of halogen, (C₁-C₄)-alkyl and (C₁-C₄)-haloalkyl,
and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen and (C₁-C₆)-alkyl,
or
R⁵ is a 5- or 6-membered heteroaromatic radical having 1 to 3 ring heteroatoms from the group consisting of N, O and S,
which is unsubstituted or substituted by one or more radicals selected from the group consisting of halogen, hydroxyl, amino, nitro, carboxyl, cyano, carbamoyl, (C₁-C₆)-alkyl, (C₂-C₄)-alkenyl, (C₂-C₄)-alkynyl, (C₁-C₆)-haloalkyl, (C₁-C₄)-alkoxy-(C₁-C₄)-alkyl, (C₁-C₄)-alkylthio-(C₁-C₄)-alkyl, mono- and di-[(C₁-C₄)-alkyl]amino-(C₁-C₄)-alkyl, hydroxy-(C₁-C₄)-alkyl, carboxy-(C₁-C₄)-alkyl, cyano-(C₁-C₄)-alkyl, (C₁-C₆)-alkoxy, which may optionally also be halogenated, (C₁-C₄)-alkoxy-(C₁-C₄)-alkoxy, (C₁-C₆)-alkylthio, [(C₁-C₆)-alkoxy]carbonyl, [(C₁-C₆)-haloalkoxy]carbonyl, (C₁-C₆)-alkanoyl, (C₁-C₆)-haloalkanoyl, mono- and di-[(C₁-C₄)-alkyl]aminocarbonyl, mono- and di-[(C₁-C₆)acyl]amino, mono- and di-[(C₁-C₄)-alkyl]amino, N—[(C₁-C₆)-acyl]-N—[(C₁-C₆)-alkyl]amino, (C₁-C₆)-alkylsulfinyl, (C₁-C₆)-haloalkylsulfinyl, (C₁-C₆)-alkylsulfonyl, (C₁-C₆)-haloalkylsulfonyl, (C₁-C₄)-alkylsulfonyloxy, (C₃-C₆)-cycloalkyl, (C₃-C₆)-cycloalkyloxy, phenyl and phenoxy,
where each of the 4 lastmentioned radicals is unsubstituted or substituted by one or more radicals selected from the group consisting of halogen, (C₁-C₄)-alkyl and (C₁-C₄)-haloalkyl,
and where two adjacent substituents may form a fused-on 5- or 6-membered ring which is carbocyclic or may also contain 1 to 3 ring heteroatoms selected from the group consisting of N, O and S and which is unsubstituted or substituted by one or more radicals selected from the group consisting of halogen and (C₁-C₆)-alkyl.

14. The compound or the salt thereof as claimed in claim 1 wherein
(R⁶)ₙ is n substituents R⁶, where R⁶, in the case that n=1, or each of the substituents R⁶ independently of one another, in the case that n is greater than 1, is a radical halogen, cyano, (C₁-C₄)-alkyl, (C₁-C₄)-haloalkyl, phenyl-(C₁-C₄)alkyl, (C₃-C₆)-cycloalkyl-(C₁-C₄)alkyl, (C₁-C₄)-alkyl which is substituted by saturated or partially saturated heterocyclyl having 3 to 6 ring atoms and one to three ring heteroatoms selected from the group consisting of N, O and S, (C₁-C₄)-alkoxy, (C₁-C₄)-alkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkoxycarbonyl, (C₃-C₆)-cycloalkyl or saturated or partially saturated heterocyclyl having 3 to 6 ring atoms and one to three ring heteroatoms selected from the group consisting of N, O and S, and
n is 0, 1, 2 or 3.

15. A process for preparing a compound of the formula (I) as defined in claim 1 or a salt thereof wherein
(a) a compound of the formula (II)
H₂N—NH-Q (II)
in which Q is a radical of the formula (Q) 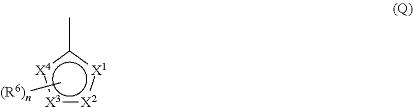
in which X¹, X², X³, X⁴, (R⁶)ₙ and n are as defined for formula (I)
is reacted with a compound of the formula (III) 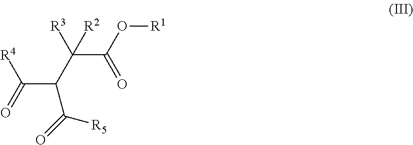
in which R¹, R², R³, R⁴ and R⁵ are defined as in formula (I)
to give the compound of the formula (I) or a salt thereof, or
(b) if R¹ in formula (I) is different from hydrogen, a compound of the formula (I′) 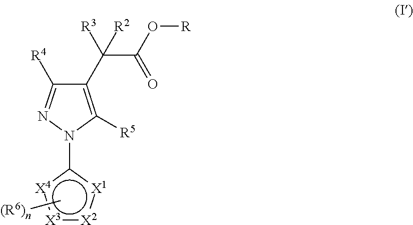
in which R², R³, R⁴, R⁵, R⁶, X¹, X², X³, X⁴ and n are as defined for formula (I) and
R is a radical which is different from the radical R¹, different from hydrogen and is selected from the group of radicals as defined for R¹, or an anhydride, acid halide or an activated ester of the compound of the formula (I′) in which R═H,
is reacted with a compound of the formula (IV)
R¹—OH (IV)
in which R¹ is as defined for formula (I)
to give the compound of the formula (I) or
(c) if R¹ in formula (I) is different from hydrogen, a compound of the formula (I″) 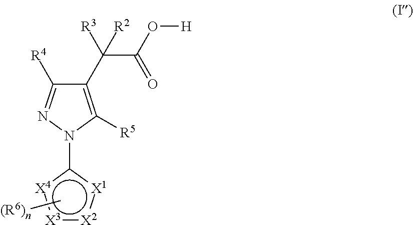
in which R², R³, R⁴, R⁵, R⁶, X¹, X², X³, X⁴ and n are as defined for formula (I) is, if appropriate after activation of the acid group, reacted with a compound of the formula (IV)
R¹—OH (IV)
in which R¹ is as defined for formula (I)
to give the compound of the formula (I) or
(d) in the case that the compound of the formula (I) in which R¹═H or a salt thereof is prepared, a compound of the formula (I′) mentioned is hydrolyzed to give the compound of the formula (I) or a salt thereof,
or
(e) a compound of the general formula (XI) is reacted with a boron derivative of the formula (XII), in the presence of a Cu(I) or Cu(II) salt and an organic base, if appropriate in a solvent, 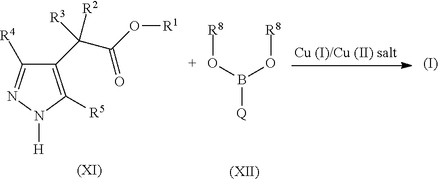
where in formula (XI) R¹, R², R³, R⁴, R⁵ have the meaning given for formula (I), and in formula (XII) R⁸ is H or (C₁-C₆)-alkyl, or both alkyl radicals R⁸ are attached cyclically to one another, and Q is a radical of the formula (Q) 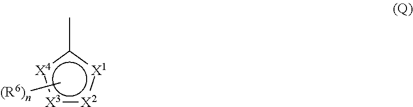
in which X¹, X², X³, X⁴, (R⁶)ₙ and n are as defined for formula (I)
or
(f) a compound of the general formula (XI) is reacted with a compound of the formula (XIII) in the presence of a suitable catalyst/ligand system with a suitable base and in a suitable solvent to give the compound of the formula (I) or a salt thereof, 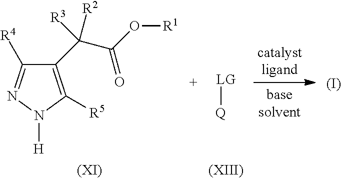
where in formula (XI) R¹, R², R³, R⁴, R⁵ have the meaning given for formula (I) and in formula (XIII) LG is a leaving group and Q is a radical of the formula (Q) 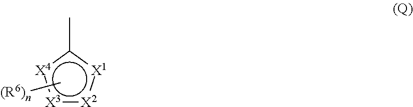
in which X¹, X², X³, X⁴, (R⁶)ₙ and n are as defined for formula (I),
or
(g) a compound of the general formula (XV) is reacted with a compound of the formula (III) in the presence of an acid, if appropriate in a solvent, to give the compound of the formula (I) or a salt thereof, 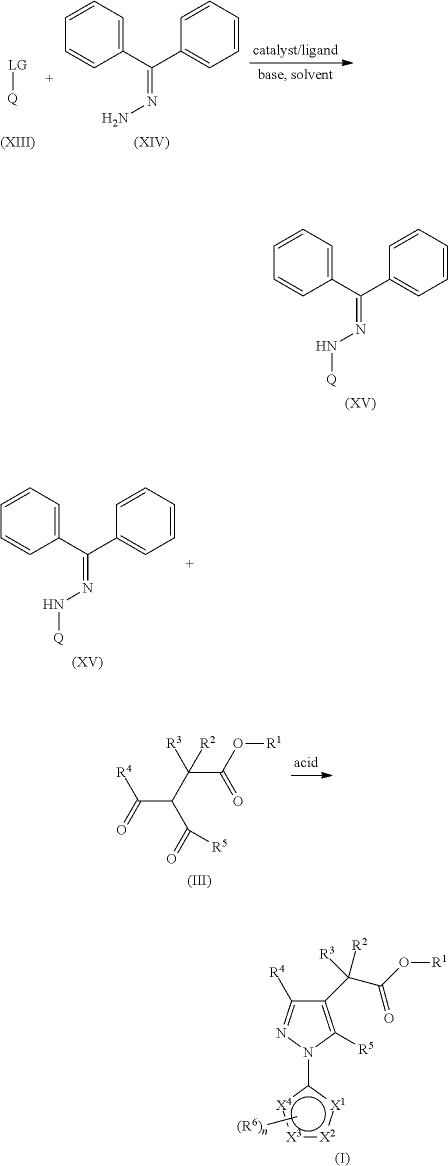
where in the formulae (XV) and (III) R¹, R², R³, R⁴, R⁵ are as defined for formula (I), LG is a leaving group and Q is a radical of the formula (Q) 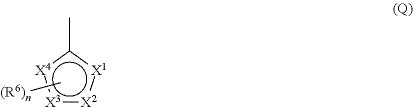
in which X¹, X², X³, X⁴, (R⁶)ₙ and n are as defined for formula (I),
or
(h) a compound of the formula (XVI) where R⁶ is as defined for formula (I) is reacted with di-tert-butyl azodicarboxylate (DBAD, XVII) in the presence of a copper salt, if appropriate in a solvent, to give a compound of the formula (XVIII) which is subsequently converted via a compound of the formula (II) or a salt thereof and according to process (a) into the compound of the formula (I): 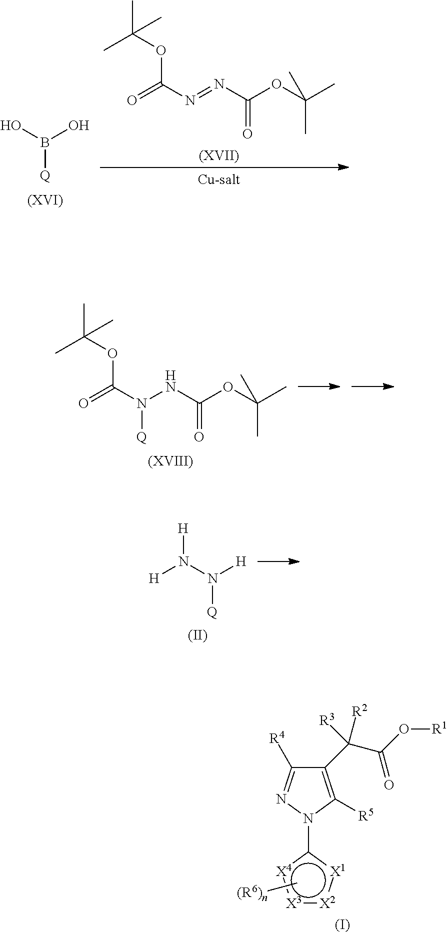
where in the formulae (XVI), (XVIII) and (II) the radical Q is a radical of the formula (Q) 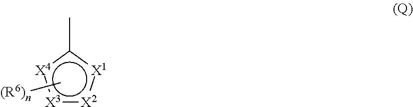
in which X¹, X², X³, X⁴, (R⁶)ₙ and n are as defined for formula (I),
or
(i) a compound of the formula (XIX), where LG′ is a leaving group, is reacted with a metal or transmetallation reagent to give a compound of the formula (XX) which for its part is reacted with di-t-butyl-azo-dicarboxylate (XVII) in the presence of a solvent to give a compound of the formula (XVIII), which is subsequently converted via a compound of the formula (II) or a salt thereof, according to process (a), into the compound of the formula (I): 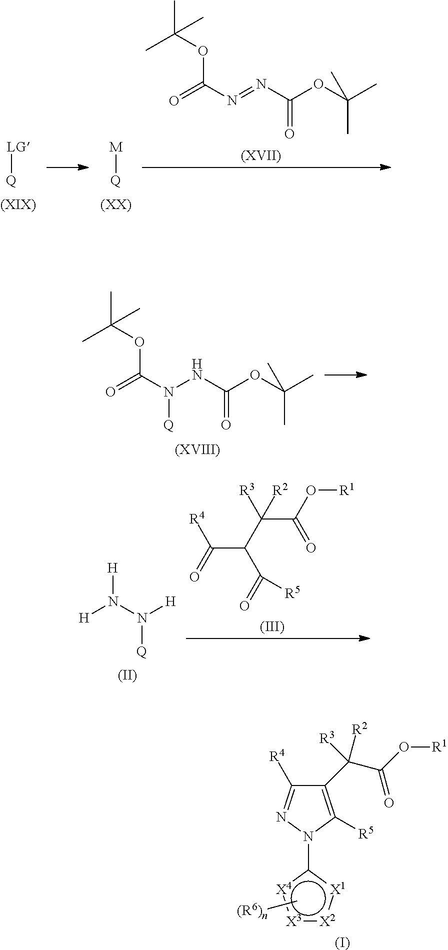
where in the formula (XIX), (XX) and (II) the group Q is a radical of the formula (Q) 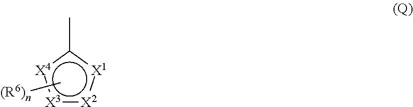
in which X¹, X², X³, X⁴, (R⁶)ₙ and n are as defined in formula (I) and where R¹, R², R³, R⁴ and R⁵ in formula (III) are defined as in formula (I).

16. A herbicidal or plant growth-regulating composition which comprises one or more compounds of the formula (I) or salts thereof as defined in claim 1 and formulation auxiliaries customary in crop protection.

17. A method for controlling harmful plants or for regulating the growth of plants which comprises applying an effective amount of one or more compounds of the formula (I) or salts thereof as defined in claim 1 onto the plants, plant seeds, the soil in which or on which the plants grow or the area under cultivation.

18. The method as claimed in claim 17 wherein the compounds of the formula (I) or salts thereof are employed for controlling harmful plants or for regulating the growth in crops of useful plants or ornamental plants.

19. The method as claimed in claim 18 wherein the crop plants are transgenic crop plants.
